SEQUENCE LISTING (681 sequences)

SEQ ID NO: 1 - DNA, 544 bp, human
tagtcttggggcaggatgattttggcctcattactttaccacccccacacctggaaagcatatactatattacaaaatgacattttgccaaaattattaatataagaagctttcagtattagtgatgtcatctgtcactataggtcatacaatccattcttaaagtacttgttatttgtttttattattactgtttgtcttctccccagggttcagtccctcaaggggccatcctgtcccaccatgcagtgccccctagcttagagcctccctcaattccccctggccaccaccccccactctgtgcctgaccttgaggagtcttgtgtgcattgctgtgaattagctcacttggtgatatgtcctatattggctaaattgaaacctggaattgtggggcaatctattaatagctgccttaaagtcagtaacttacccttagggaggctgggggaaaaggttagattttgtattcaggggttttttgtgtactttttgggtttttaaaaaattgtttttggaggggtttatgctcaatccatgt

SEQ ID NO: 2 - DNA, 530 bp, human
gatagcctcccactaggactgggaggagaataacccaggtcttaaggaccccaaagtcaggatgttgtttgatcttctcaaacatctagttccctgcttgatgggaggatcctaatgaaatacctgaaacatatattggcatttatcaatggctcaaatcttcatttatctctggccttaaccctggctcctgaggctgcggccagcagagcccaggccagggctctgttcttgccacacctgcttgatcctcagatgtggagggaggtaggcactgcctcagtcttcatccaaacacctttccctttgccctgagacctcagaatcttccctttaacccaagaccctgcctcttccactccacccttctccagggacccttagatcacatcactccacccctgccaggccccaggttaggaatagtggtgggaggaaggggaaagggctgggcctcaccgctcccagcaactgaaaggacaacactatctggagccacccactgaaagggctgcaggcatgggctgtac

SEQ ID NO: 3 - DNA, 416 bp, human
gttggttcaaacttttgggagcacggactgtcagttctctgggaagtggtcagcgcatcctgcagggcttctcctcctctgtcttttggagaaccagggctcttctcaggggctctagggactgccaggctgtttcagccaggaaggccaaaatcaagagtgagatgtagaaagttgtaaaatagaaaaagtggagttggtgaatcggttgttctttcctcacatttggatgattgtcataaggtttttagcatgttcctccttttcttcaccctcccctttgttcttctattaatcaagagaaacttcaaagttaatgggatggtcggatctcacaggctgagaactcgttcacctccaagcatttcatgaaaaagctgcttcttattaatcatacaaactctcaccatgatgtg

SEQ ID NO: 4 - DNA, 511 bp, human
aacactgcataacccgtttctttgaggagtgtgaccccaacaaggataagcacatcaccctgaaggagtggggccactgctttggaattaaagaagaggacatagatgaaaatctcttgttttgaacgaagattttaaagaactcaactttccagcatcctcctctgttctaaccacttcagaaatatatgcagctgtgatacttgtagatttatatttagcaaaatgttagcatgtatgacaagacaatgagagtaattgcttgacaacaacctatgcaccaggtatttaacattaactttggaaacaaaaatgtacaattaagtaaagtcaacatatgcaaaatactgtacattgtgaacagaagtttaattcatagtaatttcactctctgcattgacttatgagataattaatgattaaactattaatgataaaaataatgcatttgtattgttcataatatcatgtgcacttcaagaaaatggaatgctactcttttgtggtttac

SEQ ID NO: 5 - DNA, 456 bp, human
ggccgacaagaagaaggtgctggacaagtgtcaagaggtcatctcgtggctggacgccaacaccttggccgagaaggacgagtttgagcacaagaggaaggagctggagcaggtgtgtaaccccatcatcagcggactgtaccagggtgccggtggtcccgggcctgggggcttcggggctcagggtcccaagggagggtctgggtcaggccccaccattgaggaggtagattaggggcctttccaagattgctgtttttgttttggagcttcaagactttgcatttcctagtatttctgtttgtcagttctcaatttcctgtgtttgcaatgttgaaattttttggtgaagtactgaacttgctttttttccggtttctacatgcagagatgaatttatactgccatcttacgactatttcttctttttaatacacttaactcaggccatttttt

SEQ ID NO: 6 - DNA, 524 bp, human
tcctgtcacccattttgaagagtggcagaacttgaagttcaacttcctctgtaaatatccaagtataaagcccaggaacttctagaataacccagatgcgctttaattttttttaatatgttttgatcacagaacttctagaataacccagatgctctttcatattcttttaatacatcttgatcacagctgggggaaaaaaagctttttaattctgtaccttcctagtagataagtgaagagcagggaaagagacctttaaatattttgctataaaaaaatttgtgataagtttctatcaaaatggggagattgcagaaaaggcttcccttggctcccaaggaggtgtagcaggtgtgagcaatattagtgccatgtgcctttcacacagggtttgcatttatcagtctgttttccgatgatgtgtacatgaaagagtacaccatgtgaagagaagagagaatgattgaaaatgttttagtatagaactcttcttgcagtgggttgctattttctagatttta

SEQ ID NO: 7 - DNA, 562 bp, human
gtttgtagactctctgaccaaggccacctgtgccccccagcatggggccccgggtcctgggcctgctgacgccagcaaggtggtggccaagggcctggggctgagcaaggcctacgtaggccagaagagcagcttcacagtagactgcagcaaagcaggcaacaacatgctgctggtgggggttcatggcccaaggaccccctgcgaggagatcctggtgaagcacgtgggcagccggctctacagcgtgtcctacctgctcaaggacaagggggagtacacactggtggtcaaatgggggcacgagcacatcccaggcagcccctaccgcgttgtggtgccctgagtctggggcccgtgccagccggcagcccccaagcctgccccgctacccaagcagccccgccctcttcccctcaaccccggcccaggccgccctggccgcccgcctgtcactgcagctgcccctgccctgtgccgtgctgcgctcacctgcctccccagccagccgctgacctctcggctttcacttgggcagagggagccatttggtggcgctgct

SEQ ID NO: 8 - DNA, 521 bp, human
cctcacccagattgaaactctcttcaagtctaaggactatgagttcatgtggaaccctcacctgggctacatcctcacctgcccatccaacctgggcaccgggctgcgggcaggtgtcgatatcaagctgcccaacctgggcaagcatgagaagttctcggaggtgcttaagcggctgcgacttcagaagcgaggcacaggcggtgtggacacggctgcggtgggcggggtcttcgacgtctccaacgctgaccgcctgggcttctcagaggtggagctggtgcagatggtggtggacggagtgaagctgctcatcgagatggaacagcggctggagcagggccaggccatcgacgacctcatgcctgcccagaaatgaagcccggcccacacccgacaccagccctgctgcttcctaacttattgcctgcagtgcccaccatgcacccctcgatgttgccgtctggcgagcccttagccttgctgtaaggaaggcttccgtcacccttggtagagtttat

SEQ ID NO: 9 - DNA, 381 bp, human
ctctcttagctcagttactcaattcatacgtagtattttttaaaataattttatatctgtgtaccaccccatatatttcatattactgtttcacatgtacagctttctacttctttgtaagaacaccaaccaaccaaggtttaagtgattaataggcttgagcaccgggtggcagatgttctatgcagtgtggttcaagtttctttgaccgcacttatatgcattgctaatatggaatttaagataccatacacagtctctcatggacctatctctattgtagaattatgacttatgtcttacttggcaaatttttctgaatgtgacctttttttgctgatttgctgggtttgggattaactagcattattttgccacctt

SEQ ID NO: 10 - DNA, 523 bp, human
aagagttacgagttgcctgatgggcaagtgatcaccatcggaaatgaacgtttccgctgcccagagaccctgttccagccatccttcatcgggatggagtctgctggcatccatgaaaccacctacaacagcatcatgaagtgtgatattgacatcaggaaggacctctatgctaacaatgtcctatcagggggcaccactatgtaccctggcattgccgaccgaatgcagaaggagatcacggccctagcacccagcaccatgaagatcaagatcattgcccctccggagcgcaaatactctgtctggatcggtggctccatcctggcctctctgtccaccttccagcagatgtggatcagcaaacaggaatacgatgaagccgggccttccattgtccaccgcaaatgcttctaaaacactttcctgctcctctctgtctctagcacacaactgtgaatgtcctgtggaattatgccttcagttcttttccaaatcattcctagccaaagctctgactcgt

SEQ ID NO: 11 - DNA, 513 bp, human
gacgaccagccaggatatgctctcaatcatggagaaattggaattcttcgatttttcttatgaccttaacctgtgtgggctgacagaggacccagatcttcaggtttctgcgatgcagcaccagacagtgctggaactgacagagactggggtggaggcggctgcagcctccgccatctctgtggcccgcaccctgctggtctttgaagtgcagcagcccttcctcttcgtgctctgggaccagcagcacaagttccctgtcttcatggggcgagtatatgaccccagggcctgagacctgcaggatcaggttagggcgagcgctacctctccagcctcagctctcagttgcagccctgctgctgcctgcctggacttgcccctgccacctcctgcctcaggtgtccgctatccaccaaaagggctcctgagggtctgggcaagggacctgcttctattagcccttctccatggccctgccatgctctccaaaccactttttgcagctttctc

SEQ ID NO: 12 - DNA, 360 bp, human
gcaataactctgggaggggctcgagagggctggtccttatttatttaacttcacccgagttcctctgggtttctaagcagttatggtgatgacttagcgtcaagacatttgctgaactcagcacattcgggaccaatatatagtgggtacatcaagtccatctgacaaaatggggcagaagagaaaggactcagtgtgtgatccggtttctttttgctcgcccctgttttttgtagaatctcttcatgcttgacatacctaccagtattattcccgacgacacatatacatatgagaatataccttatttatttttgtgtaggtgtctgccttcacaaatgtcattgtctactcctagaa

SEQ ID NO: 13 - DNA, 453 bp, human
gcatcctcaaacatggcgccaaggataaacacgactaggccatccccagccccctgacacccagcccccgccagtcacccctccccgcacacacccgtccataccagctccctgcccatgaccctcgctcagggatccccctttgagggttagggtcccagttcccagtggaagaaacaggccaggagagtgcgtgccgagctgaggcagatgttcccacagtgaccccagagccctgggctatagtctctgacccctccaaggaaagaccaccttctggggacatgggctggagggcaggacctagaggcaccaagggaaccgcattccggggctgttccccgaggaggaagggaagcctctgtgtgccccccaggaggaagaggccctgagtcctgggatcagacaccccttcacgtgtatcccacacaaatgcaagctcaccaaggtccc

SEQ ID NO: 14 - DNA, 436 bp, human
tatagctctctttagctcaaccactctgtccatccagccaatggatgtccttccctgtacccaattcaagcttattttagggaagccttgaaactaccatgtatctggctctagctgagttattgaggattgagccagtgcaacgttaaactcagtgcacttacatttgatttaaatgatggttttatctgttgtgtgaagtggttcacccttgaggaccaggagcctccatatcctgactgaaaaccttttctgagacttagagtaacagtacttttggttccttgagttctcctgtctccagatacctaaatgaccttgacttttctgccttgtgaattcgtagtccaatcagctgaaattaaatcacttgggagggacgcatagaaggagctctaggaacacagtgccagtgcagaagtttctccaggtggcc

SEQ ID NO: 15 - DNA, 525 bp, human
ctcagagccacccctaaagagatcctttgatattttcaacgcagccctgctttgggctgccctggtgctgccacacttcaggctcttctcctttcacaaccttctgtggctcacagaacccttggagccaatggagactgtctcaagagggcactggtggcccgacagcctggcacagggcagtgggacagggcatggccaggtggccactccagacccctggcttttcactgctggctgccttagaacctttcttacattagcagtttgctttgtatgcactttgtttttttctttgggtcttgttttttttttccacttagaaattgcatttcctgacagaaggactcaggttgtctgaagtcactgcacagtgcatctcagcccacatagtgatggttcccctgttcactctacttagcatgtccctaccgagtctcttctccactggatggaggaaaaccaagccgtggcttcccgctcagccctccctgcccctcccttcaaccattccccatgggaaat

SEQ ID NO: 16 - DNA, 398 bp, human
ctcctggactcaatcatggcttgtggtctggtcgccagcaacctgaatctcaaacctggagagtgccttcgagtgcgaggcgaggtggctcctgacgctaagagcttcgtgctgaacctgggcaaagacagcaacaacctgtgcctgcacttcaaccctcgcttcaacgcccacggcgacgccaacaccatcgtgtgcaacagcaaggacggcggggcctgggggaccgagcagcgggaggctgtctttcccttccagcctggaagtgttgcagaggtgtgcatcaccttcgaccaggccaacctgaccgtcaagctgccagatggatacgaattcaagttccccaaccgcctcaacctggaggccatcaactacatggcagctgacggtgacttcaa

SEQ ID NO: 17 - DNA, 489 bp, human
ggcagcattcaagtccgatggttcctgaatggacaggaggaaacagctggggtcgtgtccaccaacctgatccgtaatggagactggaccatccagatcctggtgatgctggaaatgaccccccagcagggagacgtctacatctgccaagtggagcacaccagcctggatagttccgtcaccgtggagtggaaggcacagtctgattctgcccagagtaagacattgacgggagctgggggcttcgtgctggggctcatcatctgtggagtgggactcttcatgcacaggaggagcaagaaagttcaacgaggatctgcataaacagggttcctgacctcaccgaaaagactaatgtgccttagaacaagcatttgctgtgttttgttaacacctggttccaggacagaccctcagcttcccaagaggatactgctgccaagaagttgctctgaagtcagtttctatcgttctgctctttgattcaaa

SEQ ID NO: 18 - DNA, 459 bp, human
agaggcgggatactttcagctttccatgtaactgtatgcataaagccaatgtagtccagtttctaagatcatgttccaagctaactgaatcccacttcaatacacactcatgaactcctgatggaacaataacaggcccaagcctgtggtatgatgtgcacacttgctagactcagaaaaaatactactctcataaatgggtgggagtattttggtgacaacctactttgcttggctgagtgaaggaatgatattcatatattcatttattccatggacatttagttagtgctttttatataccaggcatgatgctgagtgacactcttgtgtatatttccaaatttttgtatagtcgctgcacatatttgaaatcatatattaagactttccaaagatgaggtccctggtttttcatggcaacttgatcagtaaggatttcacctctgtttgtaacta

SEQ ID NO: 19 - DNA, 493 bp, human
gactttttggaatagccctgtctagggcaaactgtggcccccaggagacactacccttccatgccccagacctctgtcttgcatgtgacaattgacaatctggactaccccaagatggcacccaagtgtttggcttctggctacctaaggttaacatgtcactagagtatttttatgagagacaaacattataaaaatctgatggcaaaagcaaaacaaaatggaaagtaggggaggtggatgtgacaacaacttccaaattggctctttggaggcgagaggaaggggagaacttggagaatagtttttgctttgggggtagaggcttcttagattctcccagcatccgcctttccctttagccagtctgctgtcctgaaacccagaagtgatggagagaaaccaacaagagatctcgaaccctgtctagaaggaatgtatttgttgctaaatttcgtagcactgtttacagttttcctccatgttatttatg

SEQ ID NO: 20 - DNA, 300 bp, human
gccacgattggagaatactttgcttcatagtattggagcacatgttactgcttcattttggagcttgtggagttgatgactttctgttttctgtttgtaaattatttgctaagcatattttctctaggcttttttccttttggggttctacagtcgtaaaagagataataagattagttggacagtttaaagcttttattcgtcctttgacaaaagtaaatgggagggcattccatcccttcctgaagggggacactccatgagtgtctgtgagaggcagctatctgcactctaaactgc

SEQ ID NO: 21 - DNA, 511 bp, human
ttaggtcaagttcatagtttctgtaattggcttttgaatcaaagaatagggagacaatctaaaaaatatcttaggttggagatgacagaaatatgattgatttgaagtggaaaaagaaattctgttaatgttaattaaagtaaaattattccctgaattgtttgatattgtcacctagcagatatgtattacttttctgcaatgttattattggcttgcactttgtgagtatctatgtaaaaatatatatgtatataaaatatatattgcataggacagacttaggagttttgtttagagcagttaacatctgaagtgtctaatgcattaacttttgtaaggtactgaatacttaatatgtgggaaacccttttgcgtggtccttaggcttacaatgtgcactgaatcgtttcatgtaagaatccaaagtggacaccattaacaggtctttgaaatatgcatgtactttatattttctatatttgtaactttgcatgttcttgttttgtta

SEQ ID NO: 22 - DNA, 299 bp, human
caccaaattacctaggctgaggttagagagattggccagcaaaaactgtgggaagatgaactttgtcattatgatttcattatcacatgattatagaaggctgtcttagtgcaaaaaacatacttacatttcagacatatccaaagggaatactcacattttgttaagaagttgaactatgactggagtaaaccatgtattcccttatcttttactttttttctgtgacatttatgtctcatgtaatttgcattactctggtggattgttctagtactgtattgggcttcttcgttaat

SEQ ID NO: 23 - DNA, 280 bp, human
accagctctaggtccaattgttctgctctaactgatacctcaaccttggggccagcatctcccactgcctccaaatattagtaactatgactgacgtccccagaagtttctgggtctaccacactccccaaccccccactcctacttcctgaagggccctcccaaggctacatccccaccccacagttctccctgagagagatcaacctccctgagatcaaccaaggcagatgtgacagcaagggccacggaccccatggcaggggtggcgtcttcatga

SEQ ID NO: 24 - DNA, 428 bp, human
  misc_feature (110)..(110): n is a, c, g, or t
  misc_feature (143)..(143): n is a, c, g, or t
  misc_feature (146)..(146): n is a, c, g, or t
  misc_feature (166)..(166): n is a, c, g, or t
  misc_feature (179)..(179): n is a, c, g, or t
tgtggatgtttccaagcctgacctcacggctgccctgcgtgacgtacgtcagcaatatgaaagtgtggctgccaagaacctgcaggaggcagaagaatggtacaaatccnagtttgctgacctctctgaggctgccaaccggnacnatgacgccctgcgccaggcnaagcaggagtccnctgagtaccggagacaggtgcagtccctcacctgtgaagtggatgcccttaaaggaaccaatgagtccctggaacgccagatgcgtgaaatggaagagaactttgccgttgaagctgctaactaccaagacactattggccgcctgcaggatgagattcagaatatgaaggaggaaatggctcgtcaccttcgtgaataccaagacctgctcaatgttaagatggcccttgacattgagattgccacct

SEQ ID NO: 25 - DNA, 362 bp, human
gatccagaaatacttaacacgtgaatattttgctaaaaaagcatatataactattttaaatatccatttatcttttgtatatctaagactcatcctgatttttactatcacacatgaataaaggcctttgtatctttctttctctaatgttgtatcatactcttctaaaacttgagtggctgtcttaaaagatataaggggaaagataatattgtctgtctctatattgcttagtaagtatttccatagtcaatgatggtttaataggtaaaccaaaccctataaacctgacctcctttatggttaatactattaagcaagaatgcagtacagaattggatacagtacggatttgtccaaat

SEQ ID NO: 26 - DNA, 456 bp, human
ctccagcagttctctcgaatactttgaatgttgtgtaacagttagccactgctggtgtttatgtgaacattcctatcaatccaaattccctctggagtttcatgttatgcctgttgcaggcaaatgtaaagtctagaaaataatgcaaatgtcacggctactctatatacttttgcttggttcattttttttcccttttagttaagcatgactttagatgggaagcctgtgtatcgtggagaaacaagagaccaactttttcattccctgcccccaatttcccagactagatttcaagctaattttctttttctgaagcctctaacaaatgatctagttcagaaggaagcaaaatcccttaatctatgtgcaccgttgggaccaatgccttaattaaagaatttaaaaaagttgtaatagagaatatttttggcattcctctcaatgttgtgtgtt

SEQ ID NO: 27 - DNA, 421 bp, human
caagtgcagcgatggggagcgggcccgggcggagctcaatgacaaagtccacaagctgcagaatgaagttgagagcgtcacagggatgcttaacgaggccgaggggaaggccattaagctggccaaggacgtggcgtccctcagttcccagctccaggacacccaggagctgcttcaagaagaaacccggcagaagctcaacgtgtctacgaagctgcgccagctggaggaggagcggaacagcctgcaagaccagctggacgaggagatggaggccaagcagaacctggagcgccacatctccactctcaacatccagctctccgactcgaagaagaagctgcaggactttgccagcaccgtggaagctctggaagaggggaagaagaggttccagaaggagatcgagaacctcacccag

SEQ ID NO: 28 - DNA, 563 bp, human
  misc_feature (297)..(298): n is a, c, g, or t
tcaagtccaagttcaagtccaccatcgcggcgctggaggccaagattgcacagctggaggagcaggtcgagcaggaggccagagagaaacaggcggccaccaagtcgctgaagcagaaagacaagaagctgaaggaaatcttgctgcaggtggaggacgagcgcaagatggccgagcagtacaaggagcaggcagagaaaggcaatgccagggtcaagcagctcaagaggcagctggaggaggcagaggaggagtcccagcgcatcaacgccaaccgcaggaagctgcagcgggagnnggatgaggccacggagagcaacgaggccatgggccgcgaggtgaacgcactcaagagcaagctcagagggccccccccacaggaaacttcgcagtgatgcaccaggcgaggaaacgagacctctttcgttccttctagaaggtctggaggacgtagagttattgaaaatgcagatggttctgaggaggaaacggacactcgagacgcagacttcaatggaaccaaggccagtgaataagcaactttctacagttttgcaccacgg

SEQ ID NO: 29 - DNA, 539 bp, human
tgagccaggaagaacgctccagcccaggacttcgaggctgcaatgagctataattgcatcattgcactccagcctgggcaacagagaccctgtctcaaccaccaccaccaccaccacccctactacccctgtattcaaggtaaaaattgaagtttgtatgatgtaagagatgagaaaaacccaacaggaaacacagacacatcctccagttctatcaatggattgtgcagacactgagtttttagaaaaacatatccacggtaaccggtccctggcaattctgtttacatgaaatggggagaaagtcaccgaaatgggtgccgccggcccccactcccaattcattccctaacctgcaaacctttccaacttctcacgtcaggcctttgagaattctttccccctctcctggtttccacacctcagacacgcacagttcaccaagtgccttctgtagtcacatgaattgaaaaggagacgctgctcccacggaggggagcaggaatgctgcactgtttacaccctgactgtgcttaaaa

SEQ ID NO: 30 - DNA, 202 bp, human
tgaagtgcaacaaggccatcacatctggaggaatcacttaccaggatcagccctggcatgccgattgctttgtgtgtgttacctgctctaagaagctggctgggcagcgtttcaccgctgtggaggaccagtattactgcgtggattgctacaagaactttgtggccaagaagtgtgctggatgcaagaaccccatcactgg

SEQ ID NO: 31 - DNA, 357 bp, human
cagggctgtcatcaacatggatatgacatttcacaacagtgactagttgaatcccttgtaacgtagtagttgtctgctctttgtccatgtgttaatgaggactgcaaagtcccttctgttgtgattcccaggacttttcctcaagaggaaatctggatttccacctaccgcttacctgaaatgcaggatcacctacttactgtattctacattattatatgacatagtataatgagacaatatcaaaagtaaacatgtaatgacaatacatactaacattcttgtaggagtggttagagaagctgatgcctcatttctacattctgtcattagctattatcatctaacgtttcagtg

SEQ ID NO: 32 - DNA, 445 bp, human
cttgagaccaggagacgtatccagcaagcggaacctctgggaaaagcaatctgtggataaggtcacttcccccactaaggtttgagacagttccagaaagaacccaagctcaagacgcaggacgagctcagttgtagagggctaattcgctctgttttgtatttatgttgatttactaaattgggttcattatcttttatttttcaatatcccagtaaacccatgtatattatcactatatttaataatcacagtctagagatgttcatggtaaaagtactgcctttgcacaggagcctgtttctaaagaaacccatgctgtgaaatagagacttttctactgatcatcataactctgtatctgagcagtgataccaaccacatctgaagtcaacagaagatccaagtttaaaattgcctgcggaatgtgtgcagtatctagaaa

SEQ ID NO: 33 - DNA, 496 bp, human
tgttgtttctgcactttataataaagcatggaagaaattatcttagtaggcaattgtaacactttttgaaagtaacccatttcagatttgaaatactgcaataatggttgtctttaaaaaaaaaaagaatgtactgttaaggtattactttttttcatgctgatgattcatatctaaattacattattatgttagctgacagtggtactgattttttaggttggttgttttgtggatttctttagtagtgatagtagcctgaaccacattttagataactcaattatgtatgtatgtgcatacacatatacaaacacactaatggtagaatgcttttttatgtgctagactattatatttagtagtatgtcattgtaactagccaatatcacagcttttgaaaaattaaaaaatcatcatcactataatatttcatatttgccaacagaaacatggcagataggtatcaatatgttttcaatgcctgatgacctat

SEQ ID NO: 34 - DNA, 535 bp, human
ttttaccaaagcatcaatacaaccagcccaaccatcggtccacacctgggcatttggtgagaatcaaagctgaccatggatccctggggccaacggcaacagcatgggcctcacctcctctgtgatttctttctttgcaccaaagacatcagtctccaacatgtttctgttttgttgtttgattcagcaaaaatctcccagtgacaacatcgcaatagttttttacttctcttaggtggctctgggatgggagaggggtaggatgtacaggggtagtttgttttagaaccagccgtattttacatgaagctgtataattaattgtcattatttttgttagcaaagattaaatgtgtcattggaagccatccctttttttacatttcatacaacagaaaccagaaaagcaatactgtttccattttaaggatatgattaatattattaatataataatgatgatgatgatgatgaaaactaaggatttttcaagagatctttctttccaaaacatttctggacagtacctgattgt

SEQ ID NO: 35 - DNA, 436 bp, human
gagtggactattaaatgtgcctaaatgaattttgcagtaactggtattcttgggttttcctacttaatacacagtaattcagaacttgtattctattatgagtttagcagtcttttggagtgaccagcaactttgatgtttgcactaagattttatttggaatgcaagagaggttgaaagaggattcagtagtacacatacaactaatttatttgaactatatgttgaagacatctaccagtttctccaaatgccttttttaaaactcatcacagaagattggtgaaaatgctgagtatgacacttttcttcttgcatgcatgtcagctacataaacagttttgtacaatgaaaattactaatttgtttgacattccatgttaaactacggtcatgttcagcttcattgcatgtaatgtagacctagtccatcaga

SEQ ID NO: 36 - DNA, 534 bp, human
tgctgtgtgaaccgtcgtgtgagtgtggtatgcctgatcacagatggattttgttataagcatcaatgtgacacttgcaggacactacaacgtgggacattgtttgtttcttccatatttggaagataaatttatgtgtagacttttttgtaagatacggttaataactaaaatttattgaaatggtcttgcaatgactcgtattcagatgcctaaagaaagcattgctgctacaaatatttctatttttagaaagggtttttatggaccaatgccccagttgtcagtcagagccgttggtgtttttcattgtttaaaatgtcacctgtaaaatgggcattatttatgtttttttttttgcattcctgataattgtatgtattgtataaagaacgtctgtacattgggttataacactagtatatttaaacttacaggcttatttgtaatgtaaaccaccattttaatgtactgtaattaacatggttataatacgtacaatccttccctcatcccatcacacaactttttttg

SEQ ID NO: 37 - DNA, 551 bp, human
ccatccagaatctagcgctgcgcaacacaggaatggagacgcccacaggcgtgtgcgccgcactggcggcggcaggtgtgcagccccacagcctagacctcagccacaactcgctgcgcgccaccgtaaaccctagcgctccgagatgcatgtggtccagcgccctgaactccctcaatctgtcgttcgctgggctggaacaggtgcctaaaggactgccagccaagctcagagtgctcgatctcagctgcaacagactgaacagggcgccgcagcctgacgagctgcccgaggtggataacctgacactggacgggaatcccttcctggtccctggaactgccctcccccacgagggctcaatgaactccggcgtggtcccagcctgtgcacgttcgaccctgtcggtgggggtgtcgggaaccctggtgctgctccaaggggcccggggctttgcctaagatccaagacagaataatgaatggactcaaactgccttggcttcaggggagtcccgtcaggacgttgaggacttttcgaccaattcaacc

SEQ ID NO: 38 - DNA, 305 bp, human
caaatgatgtgcaaaaccttttactggttgcatggaaatcagccaagttttataatccttaaatcttaatgttcctcaaagcttggattaaatacatatggatgttactctcttgcaccaaattatcttgatacattcaaatttgtctggtaaaaaaataggtggtagatattgaggccaagaatattgcaaaatacatgaagcttcatgcacttaaagaagtatttttagaataagaatttgcatacttacctagtgaaacttttctagaattatttttcactctaagtcatgtatgtttctct

SEQ ID NO: 39 - DNA, 456 bp, human
tcagcagtatagggaccttccgcacaagctctgtgttaagattgacaataatagtggggccattttcattttagtcttttctaagagtcaaccacaggcatttaagtcagccaaagaatattgttaccttaaagcactattttatttatagatatatctagtgcatctacatctctatactgtacactcacccataattcaaacaattacaccatggtataaagtgggcatttaatatgtaaagattcaaagtttgtctttattactatatgtaaattagacattaatccactaaactggtcttcttcaagagagctaagtatacactatctggtgaaacttggattctttcctataaaagtgggaccaagcaatgatgatcttctgtggtgcttaaggaaacttactagagctccactaacagtctcataaggaggcagccatcataaccattga

SEQ ID NO: 40 - DNA, 476 bp, human
gtgccaatcctttgaatgttccacggaaacactggtggacagattctagtgctgagaagaaacacgtttggtttggagagtccatggatggtggttttcagtttagctacggcaatcctgaacttcctgaagatgtccttgatgtgcagctggcattccttcgacttctctccagccgagcttcccagaacatcacatatcactgcaaaaatagcattgcatacatggatcaggccagtggaaatgtaaagaaggccctgaagctgatggggtcaaatgaaggtgaattcaaggctgaaggaaatagcaaattcacctacacagttctggaggatggttgcacgaaacacactggggaatggagcaaaacagtctttgaatatcgaacacgcaaggctgtgagactacctattgtagatattgcaccctatgacattggtggtcctgatcaagaatttggtgtggacgttggccct

SEQ ID NO: 41 - DNA, 376 bp, human
cctggttctggaatcctcagttcaaggttatcctacgcagagagccaggtaccaatgggtgcgctgcaatccagacagtaattctgcaaactgccttgaagaaaaaggaccaatgttcgaactacttccaggtgaatccaacaagatcccccgtctgaggactgacctttttccaaagacgagaatccaggacttgaatcgtatcttcccactttctgaggactactctggatcaggcttcggctccggctccggctctggatcaggatctgggagtggcttcctaacggaaatggaacaggattaccaactagtagacgaaagtgatgctttccatgacaaccttaggtctcttgacaggaatctgccctcagac

SEQ ID NO: 42 - DNA, 482 bp, human
tatgctttaatgctgttatctatcttattgttcttgaaaatacctgcattttttggtatcatgttcaaccaacatcattatgaaattaattagattcccatggccataaaatggctttaaagaatatatatatatttttaaagtagcttgagaagcaaattggcaggtaatatttcatacctaaattaagactctgacttggattgtgaattataatgatatgccccttttcttataaaaacaaaaaaaaaataatgaaacacagtgaatttgtagagtgggggtatttgacatattttacagggtggagtgtactatatactattacctttgaatgtgtttgcagagctagtggatgtgtttgtctacaagtatgattgctgttacataacaccccaaattaactcccaaattaaaacacagttgtgctgtcaatacctcatactgctttacctttttttcctggatatctgtgtattttc

SEQ ID NO: 43 - DNA, 570 bp, human
gttgagtcgtcatcacttttcagtgatgggagagtagatggtgaaatttattagttaatatatcccagaaattagaaaccttaatatgtggacgtaatctccacagtcaaagaaggatggcacctaaaccaccagtgcccaaagtctgtgtgatgaactttctcttcatactttttttcacagttggctggatgaaattttctagactttctgttggtgtatcccccccctgtatagttaggatagcatttttgatttatgcatggaaacctgaaaaaaagtttacaagtgtatatcagaaaagggaagttgtgccttttatagctattactgtctggttttaacaatttcctttatatttagtgaactacgcttgctcattttttcttacataattttttattcaagttattgtacagctgtttaagatgggcagctagttcgtagctttcccaaataaactctaaacattaatcaatcatctgtgtgaaaatgggttggtgcttctaacctgatggcacttagctatcagaagaccacaaaaattgactcaaatctccagtattcttg

SEQ ID NO: 44 - DNA, 508 bp, human
tctcctacatccgcattgctgataccaatatcaccagcattcctcaaggtcttcctccttcccttacggaattacatcttgatggcaacaaaatcagcagagttgatgcagctagcctgaaaggactgaataatttggctaagttgggattgagtttcaacagcatctctgctgttgacaatggctctctggccaacacgcctcatctgagggagcttcacttggacaacaacaagcttaccagagtacctggtgggctggcagagcataagtacatccaggttgtctaccttcataacaacaatatctctgtagttggatcaagtgacttctgcccacctggacacaacaccaaaaaggcttcttattcgggtgtgagtcttttcagcaacccggtccagtactgggagatacagccatccaccttcagatgtgtctacgtgcgctctgccattcaactcggaaactataagtaattctcaagaaagccctcatttttataacctgg

SEQ ID NO: 45 - DNA, 499 bp, human
gtatcaggcttcaattccattatgttttaatgttgtctctgaagatgacttgtgatttttttttcttttttttaaaccatgaagagccgtttgacagagcatgctctgcgttgttggtttcaccagcttctgccctcacatgcacagggatttaacaacaaaaatataactacaacttcccttgtagtctcttatataagtagagtccttggtactctgccctcctgtcagtagtggcaggatctattggcatattcgggagcttcttagagggatgaggttctttgaacacagtgaaaatttaaattagtaacttttttgcaagcagtttattgactgttattgctaagaagaagtaagaaagaaaaagcctgttggcaatcttggttatttctttaagatttctggcagtgtgggatggatgaatgaagtggaatgtgaactttgggcaagttaaatgggacagccttccatgttcatttgtctacctcttaactga

SEQ ID NO: 46 - DNA, 453 bp, human
gttgtgcctaccactggctggcacaccagggcaatgatttccctgcagaaggaaggaaagaatgtttcacccttgcatccttcttggagaagctacagcctgtgctcagttgagtggttcacactcagactttggctttatggttttccttcctccttgtctttgccctgaccttgatcaacaggggtgaaaagaaccaccctgaggtttccatgcctctcccattttagtggtagcattttgtgtctttactccacccttcaccctagttccaccaaggttcacacaccagatgtaactgtttttcagctgagttgtatggattaacttcagtccactgtaaatacacctgggatggggtggggttggggttgtttagggagaagcagccagacttgctttgtgaactgaatgtatttttatgcaattttgagtggcctttcaaccctaaga

SEQ ID NO: 47 - DNA, 594 bp, human
  misc_feature (42)..(42): n is a, c, g, or t
tatcagtagctcgtgttatctttttatcaactgcttcccagngtcctaaaacaatagaaattttggattgaaaagttcagcataaggagtttgagtcagtaaaggatgggataaaggagtcgagatgattcaatgaaaagtatcacaaaaaagagattgatcaacaagagaaataaaaaagcccaagaggaagtggtaggggaaggaatttaagaacagcaataagtaaaactcttaagtaactccaaaaagaaaatggtacattttgccaaagaccacttatacttgagaacatggaagaatttgcctgatactctctttggggaaaagagtctctcctcttttcctcaaaccccagtacactcagcctctctgccccaccttctcctgactttgtcctcacttgcttctgcagtacattggaacctgaattgaaagaaagtcttccttgaataattggagtttgtcttgagaggcaaatatagccccaagaatcacaagattcgaggaccatgtaggtcttttacgtagcccaaatccataaattagtctcactttttgtatttatcgtttcatattaaaccctctatatca

SEQ ID NO: 48 - DNA, 335 bp, human
taacacttggctcttggtacctgtgggttagcatcaagttctccccagggtagaattcaatcagagctccagtttgcatttggatgtgtaaattacagtaatcccatttcccaaacctaaaatctgtttttctcatcagactctgagtaactggttgctgtgtcataacttcatagatgcaggaggctcaggtgatctgtttgaggagagcaccctaggcagcctgcagggaataacatactggccgttctgacctgttgccagcagatacacaggacatggatgaaattcccgtttcctctagtttcttcctgtagtactcctcttttagatcc

SEQ ID NO: 49 - DNA, 464 bp, human
cagtcactctaaatggacaccacatgaacctctgtttagaatacctacgtatgtatgcattggtttgcttgtttcttgacagtacatttttagatctggccttttcttaacaaaatctgtgcaaaagatgcaggtggatgtccctaggtctgttttcaaagaactttttccaagtgcttgttttatttattaagtgtctacctggtaaatgttttttttgtaaactctgagtggactgtatcatttgctattctaaaccattttacacttaagttaaaatagtttctcttcagctgtaaataacaggatacagaattaacaagagaaaatgtctaactttttaagaaaaaccttattttcttcggtttttgaaaaacataatggaaataaaacaggatattgacataatagcacaaaatgacactcttctaaaactaaatgggcacaagagaattttcctggga

SEQ ID NO: 50 - DNA, 499 bp, human
gtcaaaacctaattgcaattctgttaaatctaagtaatttttagacagtgtttcaccgtattatttaggatgtgaaatgccatttctttcactgattacaccatatacaggaaacaggtaaaacagtgaaaactttattgtgctggttgatgccaacttggttgaaaagctctctgcagaagaagtgatctagactgacagaagtgttgctaattacaagttgtgttctcatgacgtaattagaaagtaacttctcaaagtacaacttttatgaaaaaaataagctgttaaaaaaaggaaatcgtaggttaatttaattgggaaaatgggcaattgacagagaccattttcctaacacatatatgtgctagtactttaactttttaaaattttacttctacgttttgtaatataaaaatttctattttaagtttagaatgttatacgtaccgaaagtatgcagccaaatcgatcagatcaaaccattttacctggagtt

SEQ ID NO: 51 - DNA, 174 bp, human
agctgtgagccttggctgttggcagtgagtgagcctggctcttgtgctggatggagcccaggcgggagcggtggccctgtccctcccacctctgtgacctgaggcctacgctttggctctggagatagccccagagcagggtgttgggatactgcagggccaggactgagcccc

SEQ ID NO: 52 - DNA, 531 bp, human
ctgttagctcctcactgtggtaaatgccacacacctttaagtagataagcagacgatagttatctgttcttttgacttaatctcatttggtttgattttccctctactaaggctttcctaccttcttcaggctgcctaagacatgtaagcgaaacacttcaataattgtccatgaggagaaaaaaagcattgtcatgcatgaaggaaactgaacttgaggtggcctccttgcttgttacatacctgggtatgtgtaggcagtttagtgcatctttgcctctcagttgaaacctgtataaccctgttacaaagctgtgttgttgcttcttgtgaaggccatgatattttgttttttccccaattaattgctattgtgttattttactaacttctctctgtattttttcttgcattgacattatagacattgaggacctcatccaaacaatttaaaaatgagtgtgaagggggaacaagtcaaaatatttttaaaagatcttcaaaaataatgcctctgtctagcatgccaac

SEQ ID NO: 53 - DNA, 467 bp, human
tgtcagagttttcaacggtgcttatagctgccagctggattccaaacaggtaccccattgtctctgagctaatgtttatatttttccattcaggcaccgaaatagttaatatttaaaataagtcttcaaaagaaaacataagagattattgagttcttgggactggatcctttatttcataagttcagatcatcttaaatgaaaatgccatgattatctgcagttaagtagatgacagctattctacatcagacttgatttttgtcagctaattacataattggtaagctataattgaaaccttatggcttaaaattccttaactcctttttgattcatgtttgtagtcatgttgtcaacagaggcaaagttaagcttgatgatggttaaaatcggtttgatagcaccatgggacatttttctaacaaaaataaatgcatgaagagacatagccttttagttttgct

SEQ ID NO: 54 - DNA, 445 bp, human
aagagctatgagctgccagatgggcaggttatcaccattggcaatgagcgcttccgctgccctgagaccctcttccagccttcctttattggcatggagtccgctggaattcatgagacaacctacaattccatcatgaagtgtgacattgacatccgtaaggacttatatgccaacaatgtcctctctgggggcaccaccatgtaccctggcattgctgacaggatgcagaaggagatcacagccctggcccccagcaccatgaagatcaagattattgctcccccagagcggaagtactcagtctggatcgggggctctatcctggcctctctctccaccttccagcagatgtggatcagcaagcctgagtatgatgaggcagggccctccattgtccacaggaagtgcttctaaagtcagaacaggttctccaaggatcccc

SEQ ID NO: 55 - DNA, 525 bp, human
caactgcccttgaccggaagcatgagtatcatcttcttcctgcccctgaaagtgacccagaatttgaccttgatagaggagagcctcacctccgagttcattcatgacatagaccgagaactgaagaccgtgcaggcggtcctcactgtccccaagctgaagctgagttacgaaggcgaagtcaccaagtccctgcaggagatgaagctgcaatccttgtttgattcaccagactttagcaagatcacaggcaaacccatcaagctgactcaggtggaacaccgggctggctttgagtggaacgaggatggggcgggaaccacccccagcccagggctgcagcctgcccacctcaccttcccgctggactatcaccttaaccagcctttcatcttcgtactgagggacacagacacaggggcccttctcttcattggcaagattctggaccccaggggcccctaatatcccagtttaatattccaataccctagaagaaaacccgagggacagcagattccacag

SEQ ID NO: 56 - DNA, 444 bp, human
ctgacctgcaggacgaaaccatgaagagcctgatccttcttgccatcctggccgccttagcggtagtaactttgtgttatgaatcacatgaaagcatggaatcttatgaacttaatcccttcattaacaggagaaatgcaaataccttcatatcccctcagcagagatggagagctaaagtccaagagaggatccgagaacgctctaagcctgtccacgagctcaatagggaagcctgtgatgactacagactttgcgaacgctacgccatggtttatggatacaatgctgcctataatcgctacttcaggaagcgccgaggggccaaatgagactgagggaagaaaaaaaatctctttttttctggaggctggcacctgattttgtatccccctgtagcagcattactgaaatacataggcttatatacaatgcttctttcct

SEQ ID NO: 57 - DNA, 444 bp, human
gaagccctggaaaatcgcctgagatacagatgaagattagaaatcgcgacacatttgtagtcattgtatcacggattacaatgaacgcagtgcagagccccaaagctcaggctattgttaaatcaataatgttgtgaagtaaaacaatcagtactgagaaacctggtttgccacagaacaaagacaagaagtatacactaacttgtataaatttatctaggaaaaaaatccttcagaattctaagatgaatttaccaggtgagaatgaataagctatgcaaggtattttgtaatatactgtggacacaacttgcttctgcctcatcctgccttagtgtgcaatctcatttgactatacgataaagtttgcacagtcttacttctgtagaacactggccataggaaatgctgtttttttgtactggactttaccttgatatatgt

SEQ ID NO: 58 - DNA, 450 bp, human
gctggtatcagaacagcttccctcactgtgtacagaacgcaagaagggaataggtggtctgaacgtggtgtctcactctgaaaagcaggaatgtaagatgatgaaagagacaatgtaatactgttggtccaaaagcatttaaaatcaatagatctgggattatgtggccttaggtagctggttgtacatctttccctaaatcgatccatgttaccacatagtagttttagtttaggattcagtaacagtgaagtgtttactatgtgcaagggtattgaagttcttatgaccacagatcatcagtactgttgtctcatgtaatgctaaaactgaaatggtccgtgtttgcattgttaaaaatgatgtgtgaaatagaatgagtgctatggtgttgaaaactgcagtgtccgttatgagtgccaaaaatctgtcttgaaggcagctacactt

SEQ ID NO: 59 - DNA, 474 bp, human
aacctgaaaacatcccagccaagaactggtataggagctccaaggacaagaaacacgtctggctaggagaaactatcaatgctggcagccagtttgaatataatgtagaaggagtgacttccaaggaaatggctacccaacttgccttcatgcgcctgctggccaactatgcctctcagaacatcacctaccactgcaagaacagcattgcatacatggatgaggagactggcaacctgaaaaaggctgtcattctacagggctctaatgatgttgaacttgttgctgagggcaacagcaggttcacttacactgttcttgtagatggctgctctaaaaagacaaatgaatggggaaagacaatcattgaatacaaaacaaataagccatcacgcctgcccttccttgatattgcacctttggacatcggtggtgctgaccaggaattctttgtggacattggcccagtctgtt

SEQ ID NO: 60 - DNA, 421 bp, human
gagccattggaagactgtcctctatggcaatgatctcagggctcagtggcaggaaatcctcaacagggtcaccaaccagcccgctcaatgcagaaaaactagaatctgaagatgtgtcccaagctttccttgaggctgttgctgaggaaaagcctcatgtaaaaccctatttctctaagaccattcgcgatttagaagttgtggagggaagtgctgctagatttgactgcaagattgaaggatacccagaccccgaggttgtctggttcaaagatgaccagtcaatcagggagtcccgccacttccagatagactacgatgaggacgggaactgctctttaattattagtgatgtttgcggggatgacgatgccaagtacacctgcaaggctgtcaacagtcttggagaagccacctgcac

SEQ ID NO: 61 - DNA, 502 bp, human
ttatcaagtgtcaagatcagcaagtgtccttaagtcaaataggtttttttttgttggtggttgtgcttgctttccttttttagaaagttctagaaaataggaaaacgaaaaatttcattgagatgagtagtgcatttaattattttttaaaaaactttttaagtacttgaattttatatcaggaaaacaaagttgttgagccttgcttcttccgttttgccctttgtctcgctccttattcttttttggggggagggttatttgcttttttatcttcctggcataatttccattttattcttctgagtgtctatgttaacttccctctatcccgcttataaaaaaattctccaacaaaaatacttgttgacttgatgttttatcacttctctaagtaaggttgaaatatccttattgtagctactgtttttaatgtaaaggttaaacttgaaaagaaattcttaatcacggtgccaaaattcattttctaacaccatgtgtt

SEQ ID NO: 62 - DNA, 541 bp, human
  misc_feature (163)..(164): n is a, c, g, or t
agattctaacggtctgttctgtttcaaggcactctagattccattggtccaagattccggatcctaagcatctaagttataagactctcacactcagttgtgactaactagacaccaaagttctaataatttctaatgttggacacctttaggttctttgctnnattctgcctctctaggaccatggttaagagtccaagaatccacatttctaaaatcttatagttctaggcactgtagttctaagactcaaatgttctaagtttctaagattctaaaggtccacaggtctagactattaggtgcaatttcaaggttctaaccctatactgtagtattctttggggtgcccctctccttcttagctatcattgcttcctcctccccaactgtgggggtgtgcccccttcaagcctgtgcaatgcattagggatgcctcctttccgcaggggatggacgatctcccacctttcgggccatgttgcccccgtgagccaatccctcaccttctgagtacagagtgtggactctggtgcctcca

SEQ ID NO: 63 - DNA, 496 bp, human
ggctctggaggtgggcagcaatctgtcaatcaccttgttaaagagattgatatgctgctgaaagaatatttactctctggagacatatctgaagctgaacattgccttaaggaactggaagtacctcattttcaccatgagcttgtatatgaagctattataatggttttagagtcaactggagaaagtacatttaagatgattttggatttattaaagtccctttggaagtcttctaccattactgtagaccaaatgaaaagaggttatgagagaatttacaatgaaattccggacattaatctggatgtcccacattcatactctgtgctggagcggtttgtagaagaatgttttcaggctggaataatttggaaacaactcagagatctttgtccttcaaggggcagaaagcgttttgtaagcgaaggagatggaggtcgtcttaaaccagagagctactgaatataagaactcttgcagtcttagatgttat

SEQ ID NO: 64 - DNA, 507 bp, human
gtaagattcctcctaactttcacagtcgatgacaagattgtctttttatctgatattttgaagggtatattgctttgaagtaagtctcaataaggcaatatattttagggcatctttcttcttatctctgacagtgttcttaaaattatttgaatatcataagagccttggtgtctgtcctaattcctttctcactcaccgatgctgaatacccagttgaatcaaactgtcaacctaccaaaaacgatattgtggcttatgggtattgctgtctcattcttggtatattcttgtgttaactgcccattggcctgaaaatactcattgtaagcctgaaaaaaaaaatctttcccactgttttttctgcttgttgtaagaatcaaatgaaataatgtatgtgaaagcaccttgtaaactgtaacctatcaatgtaaaatgttaaggtgtgttgttatttcattaattacttctttgtttagaatggaatttcctatgcactactgtagc

SEQ ID NO: 65 - DNA, 450 bp, human
tacacttttttttgccaactgacttaacaacattgctgtcaggtggaaatttcaagcacttttgcacatttagttcagtgtttgttgagaatccatggcttaacccacttgttttgctatttttttctttgcttttaattttccccatctgattttatctctgcgtttcagtgacctaccttaaaacaacacacgagaagagttaaactgggttcattttaatgatcaatttacctgcatataaaatttatttttaatcaagctgatcttaatgtatataatcattctatttgctttattatcggtgcaggtaggtcattaacaccacttcttttcatctgtaccacaccctggtgaaacctttgaagacataaaaaaaacctgtctgagatgttctttctaccaatctatatgtctttcggttatcaagtgtttctgcatggtaatgtc

SEQ ID NO: 66 - DNA, 549 bp, human
  misc_feature (76)..(76): n is a, c, g, or t
aaactactaaccactgcaagctcttgtcaaattttagtttaattggcattgcttgttttttgaaactgaaattacntgagtttcattttttctttgaatttatagggtttagatttctgaaagcagcatgaatatatcacctaacatcctgacaataaattccatccgttgttttttttgtttgtttgttttttcttttcctttaagtaagctctttattcatcttatggtgcagcaattttaaaatttgaaatattttaaattgtttttgaactttttgtgtaaaatatatcagatctcaacattgttggtttcttttgtttttcattttgtacaactttcttgaatttagaaattacatctttgcagttctgttaggtgctctgtaattaacctgacttatatgtgaacaattttcatgagacagtcatttttaactaatgcagtgattctttctcactactatctgtattgtggaatgcacaaaattgtgtaggtgctgaatgctgtaaggagtttaggttgtatgaattctacaaccctataata

SEQ ID NO: 67 - DNA, 489 bp, human
gagatactatttttgctgagcaaccagtgtgtttcaggatgatacaaagaaaaatatagaatagaaataagtgcaggcttggaatcagctacaaatcctaaagatggggtgtgtgtggatgtgtgtgtgtgtgtgtgtacaccattgtgtgtttgtaaaatgtgtatgttcatgagtaagggtgtgtgtgtgtgtgtattaaaattccagagtgaccgtggcacttgggtgtacaggtaattcctccagagctgtttgctggcttcaggagtggagtgagaatttcttttttatgaaaagggatataaaggcaccgagctgatgcagtatttgtaatattaagttgacctaacaaggtatttgcatgagtcacaattacaaagttttgagcggttttgtaatttgacatttaggaaagtctcctatttattctcatactttacattcatgcttagtatactatagaggatgccagctttaatctttctg

SEQ ID NO: 68 - DNA, 543 bp, human
gctttcaattgatggactactctattttttgcaaatttgtaaactttgcttctccaaatacaagtactaggttgtccatttatggtacctatttggtgctagtaaattttcaaactagatttataaatgcactgtaatatgtacacaacttagaaaccaaattacaagtattcagttccaatacttcattaatttcaatcaaccaaagttagttcagtagcttatctcagttatgagtataatacattacatgtaaattaagtgtgtgtatactgtaatcgtgctattttttatcattgaaacatttataaactagaataataatgcccttaatgtgagggtttgtaatggtgcttattaagaccaaagacttgttaaatgtatacaccaagtggtaatgaaatttcgtgactggcccacacgtgcatagaggtctgggaggaccaggaaacagcctcagtggccagaggatcaccagtgcatccttcatcacagcatgtgcaatatgccaagattaccctcggtcattcctgtcaacaaggg

SEQ ID NO: 69 - DNA, 500 bp, human
gcgtgagtgtgtgagcgcttctgcagcctcggcctaggtcacgttggccctcaaagcgagccgttgaattggaaactgcttctagaaactctggctcagcctgtctcgggctgacccttttctgatcgtctcggcccctctgattgttcccgatggtctctctccctctgtcttttctcctccgcctgtgtccatctgaccgttttcacttgtctcctttctgactgtccctgccaatgctccagctgtcgtctgactctgggttcgttggggacatgagattttattttttgtgagtgagactgagggatcgtagatttttacaatctgtatctttgacaattctgggtgcgagtgtgagagtgtgagcagggcttgctcctgccaaccacaattcaatgaatccccgacccccctaccccatgctgtacttgtggttctctttttgtattttgcatctgaccccggggggctgggacagattggcaatgggccgtccc

SEQ ID NO: 70 - DNA, 482 bp, human
agaaagaggcgctgctcactgttttcctgcttcagtttttctcttatagtaccatcactataatcaacgaacttctcttctccacccagagatggcttttccaacacattttaattaaaggaactgagtacattaccctgatgtctaaatggaccaaagatctgagatccattgtgattatatctgtatcaggtcagcagaagaaggaactgagcagttgaactctgagttcatcaattctaatatttggaaattatctacaatggaatcttccctctgttctctgataacctacttgcttactcaatgcctttaagccaagtcaccctgttgcctatgggaggaggtggaaggatttggcaagctcaaccacatgctatttagttagcatcagttgtcaccaacagtctttctgcaaagggcaggagagctttgggggaaaggaaaaggcttaccaggctgctatggtcaactcttcagaa

SEQ ID NO: 71 - DNA, 508 bp, human
agccctggagaagacgaaagccaacatcaagtgggtgaaggagaacaaggaggtggtgctccagtggttcacagaaaacagcaaatagtccccagcccttgaagtcacccggccccgatgcaaggtgcccacatgtgtccatcccagcggctggtgcagggcctccattcctggagcccgaggcaccagtgtcctcccctcaaggacaaagtctccagcccacgttctctctgcctgtgagccagtctagttcctgatgacccaggctgcctgagcacctcccagcccctgcccctcatgccaaccccgccctaggcctggcatggcacctgtcgcccagtgccctggggctgatctcagggaagcccagctccagggccagatgagcagaagctctcgatggacaatgaacggccttgctgggggccgccctgtaccctctttcacctttccctaaagaccctaaatctgaggaatcaacagggcagcagatctgtatatttttttc

SEQ ID NO: 72 - DNA, 460 bp, human
attatctggtcttttcctgttgtgcaaaaatgactcattgctccgaatgtcaaaaacaaatgcgacaaacaatggcacttcatcatttaaagtaatgttgccaagagaaaaaatttcctgggagggaggtttcccacaagccaaatctcctaagcctcaaatgctagcactttttggcagttggataggaaatgagacattctttggcagccaaaataagagaggccgatggtgaaactttttgagacaccctatggccttcttgtcaaaaccttcactggagctcaagaaaagcatttctgttgtgttatttgcagtgcagatgatgtctgtgtaacaacataatggttattcacctttttttgattttgatttttgctgtgttatcaaaaacttgaatactgtgagaagaagtgaattttcagttgacgaatcagcatcttgttcccatggtgataac

SEQ ID NO: 73 - DNA, 551 bp, human
cttcgaccacgtgatcaccaacatgaacaacaattatgagccccgcagtggcaagttcacctgcaaggtgcccggtctctactacttcacctaccacgccagctctcgagggaacctgtgcgtgaacctcatgcgtggccgggagcgtgcacagaaggtggtcaccttctgtgactatgcctacaacaccttccaggtcaccaccggtggcatggtcctcaagctggagcagggggagaacgtcttcctgcaggccaccgacaagaactcactactgggcatggagggtgccaacagcatcttttccgggttcctgctctttccagatatggaggcctgacctgtgggctgcttcacatccaccccggctccccctgccagcaacgctcactctacccccaacaccaccccttgcccagccaatggacacagtagggcttggtgaatgctgctgagtgaatgagtaaataaactcttcaaggccaaggaacagtggtctaattcaactctgtgtcccagcactggcacaccagaagtgccatgctcagaaa

SEQ ID NO: 74 - DNA, 542 bp, human
tgatgagctttcctttgatccggacgacgtaatcactgacattgagatggtggacgagggctggtggcggggacgttgccatggccactttggactcttccctgcaaattatgtcaagcttctggagtgactagagctcactgtctactgcaactgtgatttcccatgtccaaagtggctctgctccaccccctccctattcctgatgcaaatgtctaaccagatgagtttctggacagacttccctctcctgcttcattaagggcttggggcagagacagcatggggaaggaggtccccttccccaagagtcctctctatcctggatgagctcatgaacatttctcttgtgttcctgactccttcccaatgaacacctctctgccaccccaagctctgctctcctcctctgtgagctctgggcttcccagtttgtttacccgggaaagtacgtctagattgtgtggtttgcctcattgtgctatttgcccactttccttccctgaagaaatatctgtgaaccttctttctgttcagtccta

SEQ ID NO: 75 - DNA, 436 bp, human
gctggaatacttactcttgtcgggagattgaaccactaaaatgttagagcagaattcattatgctgtggtcacaggggtgtcttgtctgagaacaaatacaattcagtcttctctttggggttttagtatgtgtcaaacataggactggaagtttgcccctgttcttttttcttttgaaagaacatcagttcatgcctgaggcatgagtgactgtgcatttgagatagttttccctattctgtggatacagtcccagagttttcagggagtacacaggtagattagtttgaagcattgaccttttatttattccttatttctctttcatcaaaacaaaacagcagctgtgggaggagaaatgagagggcttaaatgaaatttaaaataagctatattatacaaatactatctctgtattgttctgaccctggtaaa

SEQ ID NO: 76 - DNA, 379 bp, human
  misc_feature (170)..(170): n is a, c, g, or t
aatgcgaaggctaagtgtcaccccctttctctgcctctggctgggccttgctaagggccaaggaaagaaagacattttttagggggcagccagtccaaatgccaaaagaagaccagttcttgccctgattgtatgaaatttgacattttggcactttttttttttttttnggccaatcagattttctatgttctaaggacatggctgctgtagaatagcacagacgtggatgataaattatccccagaagcagcatgacagaatgcctcggggagcacttggaagggaaattgcagttctgttgaaatagaggaaaatcccttggtaaagacacagcctgttaggctcgtgtgggcctccagtatgttcaccaggggaa

SEQ ID NO: 77 - DNA, 451 bp, human
aacgatgtcacatgcgtgttcgaccccgtgcacaccatctcccacaccgtcatctcgctgcctaccttccgcgagttcacccgccctgaagagatcatcttcctccgggccatcacgccaccgcatcctgccagccaggctaacatcatcttcgacatcacggaagggaacctgcgggactcttttgacatcatcaagcgttacatggacggcatgaccgtgggtgtcgtgcgccaggtgcggcccatcgtgggcccatttcatgccgtcctgaagctggagatgaactatgtggtcgggggcgtggtttcccaccgaaatgttgtcaacgtccgcatcttcgtctctgagtactggttctgagggctggtctgccgcacagccgcaggtgcacctccaggccaaatcattgctgccagtgactgtggtctgtacttgtttataccctcag

SEQ ID NO: 78 - DNA, 500 bp, human
  misc_feature (410)..(411): n is a, c, g, or t
gacagacctgagaccaatctgggtagaagcaaaaagttgaaccttttaaagtgctgaacacaaatccaaattcgaatggttcaagcagccgtgaaatcgctcttcataaagtgggcttaattctctagtttaagttcttttgatggaatgaattaattaatgtgtcaggtggcttatttgtggatgccatgattgatgatgttcattttaagctcttacctatagtacaagtacatgatgctactgaatatttttccacttggaaactgtgagctggttgttgcattaaaacacacatacaaacaaaatcaaaaacactgcggactttcactcaagctggtctttcttccccagtgtaaggcaatcctgcctactaacaacaccaacaacaaaacactccatctgtgaanntgacgcagttaagggggctaggcagggcatttgtgccaactaagaatcaccagatacccaccataagtacctatcgcagttttgaagtc

SEQ ID NO: 79 - DNA, 480 bp, human
acctcgcaacatcaacatctatacttacgatgatatggaagtgaagcaaatcaacaaacgtgcctctggccaggcttttgagctgatcttgaagccaccatctcctatctcagaagccccacgaactttagcttctccaaagaagaaagacctgtccctggaggagatccagaagaaactggaggctgcaggggaaagaagaaagtctcaggaggcccaggtgctgaaacaattggcagagaagagggaacacgagcgagaagtccttcagaaggctttggaggagaacaacaacttcagcaagatggcggaggaaaagctgatcctgaaaatggaacaaattaaggaaaaccgtgaggctaatctagctgctattattgaacgtctgcaggaaaaggagaggcatgctgcggaggtgcgcaggaacaaggaactccaggttgaactgtctggctgaagcaagggagggtctggcacgcc

SEQ ID NO: 80 - DNA, 510 bp, human
atggcctctgtgaataatgtaactccagttacacggtgacttttaatagcatacagtgatttgatgaaaggacgtcaaacaatgtggcgatgtcgtggaaagttatctttcccgctctttgctgtggtcattgtgtcttgcagaaaggatggccctgatgcagcagcagcgccagctgtaataaaaaataattcacactatcagactagcaaggcactagaactggaaaagaccacagaaaacaaagaatccaaccctttcatcttacaggtgaacaaactgtgatgatgcacatgtatgtgttttgtaagctgtgagcaccgtaacaaaatgtaaatttgccattattaggaaagtgctggtggcagtgaagaagcacccaggccacttgactcccagtctggtgccctgtctacaccagacaacacaggagctgggtcagattcccctcagctgcttaacaaagttcctcgaacagaaagtgcttacaaagctgccttctcggatact

SEQ ID NO: 81 - DNA, 560 bp, human
agctgccttctcggatactgaaaggtcgagttttctgaactgcactgattttattgcagttgaaaaacccaaagctattccaaagatttcaagctgttctgagacatcttctgatggctttacttcctgagaggcaatgtttttactttatgcataattcattgttgccaaggaataaagtgaagaaacagcacctttttaatatataggtctctctggaagagacctaaatttagaaagagaaaactgtgacaattttcatattctcattcttaaaaaacactaatcttaactaacaaaagttcttttgagaataagttacacacaatggccacagcagtttgtctttaatagtatagtgcctatactcatgtaatcggttactcactactgcctttaaaaaaaaccagcatatttattgaaaacatgagacaggattatagtgccttaaccgatatattttgtgacttaaaaaatacatttaaaactgctcttctgctctagtaccatgcttagtgcaaatgattatttctatgtacaactgatgcttgttcttattt

SEQ ID NO: 82 - DNA, 561 bp, human
ttcagttgctaatgaggctcctccgctctggacacaacccttttatagattaatttctctgccaattaacttgtcattttcagtacatattttactattccacaccaaccataattacaacaagggatttttcttatgcactcctatgcatgtgaataacatgtggtgtaattctgcttcttacagaagtattactgaaggtattatttccaatattatttggtttattatgcggatcttttttatatatgcagtcccatcccttctgtgccactcaatgccatccagacatggtttttccctccaggggcctttctctccagagggcacttcggctgcctctgcttcctctcattcgaggcccggctcttgctgacagaataggttccgttctgggcggtggttctcgagcctgccattcaaaaccaaagcaaattggagcatttctcacaacatggtattgaagttcctttttgttctcaaaagttgtgaccgtgttaaattgtactcccttagtcctgtaaggtatgttaagtgaatcgcagttacgctgtactttta

SEQ ID NO: 83 - DNA, 458 bp, human
agaaaatttgccaatctttcctactttctatttttatgatgacaatcaaagccggcctgagaaacactatttgtgactttttaaacgattagtgatgtccttaaaatgtggtctgccaatctgtacaaaatggtcctatttttgtgaagagggacataagataaaatgatgttatacatcaatatgtatatatgtatttctatatagacttggagaatactgccaaaacatttatgacaagctgtatcactgccttcgtttatatttttttaactgtgataatccccacaggcacattaactgttgcacttttgaatgtccaaaatttatattttagaaataataaaaagaaagatacttacatgttcccaaaacaatggtgtggtgaatgtgtgagaaaaactaacttgatagggtctaccaatacaaaatgtattacgaatgcccctgttcatgtt

SEQ ID NO: 84 - DNA, 543 bp, human
ccaggactacagaataccatcccctggtaccgtgtagttgccgaagtccagatctgccatggcaaaacggaggctgtgggccaggtccacatcttcttccaggatgggatggtgacgttgactccaaacaagggtgtgtgggtgaatggtctccgagtggatctcccagctgagaagttagcatctgtgtccgtgagtcgtacacctgatggctccctgctagtccgccagaaggcaggggtccaggtgtggcttggagccaatgggaaggtggctgtgattgtcagcaatgaccatgctgggaaactgtgtggggcctgtggaaactttgacggggaccagaccaatgattggcatgactcccaggagaagccagcgatggagaaatggagagcgcaggacttctccccatgttatggctgatcagtcatccaccaggaacgaagatttcctgaagaagacctggtccctctggaggttgcggtggctgaaggatgcatcatgtgctcctaccctgctctaccgcttttctgggtcacagag

SEQ ID NO: 85 - DNA, 401 bp, human
aaagacaagcattgggtcagacccataaaccacctcccaaaggctgtcatttcattgcactggaattttgctttatcagaagcaaggaagtaagggagtcattgccttgggcctgggaatctaagtgggagacaatattaatttggatccgattaattggagattactaactgtggacaaaagtttatctttgcacaatcaataaaaatggcatttttttagtaaattaagagcataaacaatattgctagaggtggcatgtttagtctaccaaaaacaatacttttcaggcactttagaaatatccttttagaagcagcgagtgcatgggctaattatcatcaatctttatgtatttgttaaagaaacatctacaggatctttattggtgaccttttgta

SEQ ID NO: 86 - DNA, 454 bp, human
gtccttcacatcaccattttgagacctcagcttggcactcaggtgctgaagggtaatatggactcagccttgcaaatagccagtgctagttctgacccaaccacagaggatgctgacatcatttgtattatgttccaaggctactacagagaaggctgcctgctatgtatttgcaaggctgatttatggtcagaatttccctctgatatgtctagggtgtgatttaggtcagtagactgtgattcttagcaaaaaatgaacagtgataagtatactgggggcaaaatcagaatggaatgctctggtctatataaccacatttctgagcctttgagactgttcctgagccttcagcactaacctatgagggtgagctggtcccctctatatatacatcatacttaactttactaagtaatctcacagcatttgccaagtctcccaatatccaatt

SEQ ID NO: 87 - DNA, 492 bp, human
atgtgtcactgattttttagctcaaaatcatcactgttaatttccagtcaccccaaatatggttaaaagatttttttttttaatcatgaagagaaaattagtagcatttctttctctccccattatttattggttttcctcactaatctttttttttttagtccaaaagccaaaaatatttatcttggttttacattttaatttccattcttaattgtaatttttttctttaaataaggaaaccaatataatctcatgtataaaaacttaaatattttacaagttacatatagcatcattctaaaataagaattttttttgttttctgtctgcttttttcttatgtctcttgttgagttttatattttcagtggttatttttgcttgtgttagatcattattaaaatatatccaatgtccctttgatacttgtgctctgctgagaatgtacagtttgcattaaacatcccaggtctcatccttcaggaattt

SEQ ID NO: 88 - DNA, 444 bp, human
gaagcgcctggcagtgtaccaggccggggcccgcgagggcgccgagcgcggcctcagcgccatccgcgagcgcctggggcccctggtggaacagggccgcgtgcgggccgccactgtgggctccctggccggccagccgctacaggagcgggcccaggcctggggcgagcggctgcgcgcgcggatggaggagatgggcagccggacccgcgaccgcctggacgaggtgaaggagcaggtggcggaggtgcgcgccaagctggaggagcaggcccagcagatacgcctgcaggccgaggccttccaggcccgcctcaagagctggttcgagcccctggtggaagacatgcagcgccagtgggccgggctggtggagaaggtgcaggctgccgtgggcaccagcgccgcccctgtgcccagcgacaatcactgaacgccgaagcc

SEQ ID NO: 89 - DNA, 500 bp, human
actgtgatataggtactctgatttaaaactttggacatcctgtgatctgttttaaagttggggggtgggaaatttagctgactagggacaaacatgtaaacctattttcctatgaaaaaagttttaaatgtcccacttgaataacgtaattcttcatagtttttttaatctatggataaatggaaacctaattatttgtaatgaattatttagacagttctaagccctgtcttctgggagttatcaattttaaagagaacttttgtgcaattcaaatgaagtttttataagtaattgaaaatgacaacacaataacactttctgtataaaagtatatattttatgtgatttattcctactaaatgaaagtgcactactgcctcatgtaaagactcttgcacgcagagcctttaagtgactaaggaacaacatagatagtgagcatagtccccacctccacccctcacaatttatttgaatacttcaattgtgcctctcaa

SEQ ID NO: 90 - DNA, 444 bp, human
ttcagacagttatatcctccttttaaaccattgttgttgagtgtaagatgtccttcatgttttcttataaagtcagtgtttagaaatgttaccctttctaagttatatacagatcaaatgcttttttctttcacgtacatccatcatttgcaactgctgttcgtacacagaaacaggactgctcaaatgatcctatttgtattttctgatgctatcagactctaatgtttttttccctaaaatattattgccatcatgctttaggaatttttatatttttacacaatcatattttagtatggtgtctgtttatgtaactctgacttgctggaaaagttgaaactccaaataatctgaaactagaaaagaaatagcacataattactaccttccccttggcggctctcctcccccaacccccaccccacaattttatgacttcca

SEQ ID NO: 91 - DNA, 435 bp, human
caacgtctaactggacttcccaagataaatggtaccagcgtcctcttaaaagatgccttaatccattccttgaggacagaccttagttgaaatgatagcagaatgtgcttctctctggcagctggccttctgcttctgagttgcacattaatcagattagcctgattctcttcagtgaattttgataatggcttccagactctttgcgttggagacgcctgttaggatcttcaagtcccatcatagaaaattgaaacacagagttgttctgctgatagttttggggatacgtccatctttttaagggattgctttcatctaattctggcaggacctcaccaaaagatccagcctcatacctacatcagacaaaatatcgccgttgttccttctgtactaaagtattgtgttttgctttggaaacacccactcact

SEQ ID NO: 92 - DNA, 486 bp, human
gccagacgctggggccatagtgagtgtgggcacaaggaagacgctgcagtgaattgcacagatatttcagtgcagaaaaccccacaaaaagccacaacaggtcgctcatcccgtcagtcatcctttattgcagtcgggatccttggggttgttctgttggccattttcgtcgcattattcttcttgactaaaaagcgaagacagagacagcggcttgcagtttcctcaagaggagagaacttagtccaccaaattcaataccgggagatgaattcttgcctgaatgcagatgatctggacctaatgaattcctcaggaggccattctgagccacactgaaaaggaaaatgggaatttataacccagtgagttcagcctttaagataccttgatgaagacctggactattgaatggagcagaaattcacctctctcactgactattacagttgcatttttatggagttcttcttctcctaggattcc

SEQ ID NO: 93 - DNA, 522 bp, human
ttttcattgttcattgatatgctcagtatgctgccacataagatgaatttaattatattcaaccaaagcaatatactcttacatgatttctaggccccatgacccagtgtctagagacattaattctaaccagttgtttgcttttaaatgagtgatttcattttgggaaacaggtttcaaatgaatatatatacatgggtaaaattactctgtgctagtgtagtcttactagagaatgtttatggtcccacttgtatatgaaaatgtggttagaatgttaattggataatgtatatataagaagttaaagtatgtaaagtataacttcagccacatttttagaacactgtttaacatttttgcaaaaccttcttgtaggaaaagagagctctctacatgaagatgacttgttttatatttcagattttattttaaaagccatgtctgttaaacaagaaaaaacacaaaagaactccagattcctggttcatcattctgtattcttactcactttttcaagtt

SEQ ID NO: 94 - DNA, 465 bp, human
atcctcattcttatactgcttttcgtggccactttggacaagtcctggtggactctccctgggaaagagtccctgaatctctggtacgactgcacgtggaacaacgacaccaaaacatgggcctgcagtaatgtcagcgagaatggctggctgaaggcggtgcaggtcctcatggtgctctccctcattctctgctgtctctccttcatcctgttcatgttccagctctacaccatgcgacgaggaggtctcttctatgccaccggcctctgccagctttgcaccagcgtggcggtgtttactggcgccttgatctatgccattcacgccgaggagatcctggagaagcacccgcgagggggcagcttcggatactgcttcgccctggcctgggtggccttccccctcgccctggtcagcggcatcatctacatccacctacggaagcgggagtgagcgccccgc

SEQ ID NO: 95 - DNA, 560 bp, human
caagtatcggggctctgctatcaaggtgcaaagtccttcgtggatgcaacctcaaccatatattctacagcaccctggtgccgtgttaactccctcaatggagcacaccatgtcactacagcccgcatcaatgatcagccctctggcccagcagatgagtcatctgtcactaggcagcaccggaacatacatgcctgcaacgtcagctatgcaaggagcctacttgccacagtatgcacatatgcagacgacagcggttcctgttgaggaggcaagtggtcaacagcaggtggctgtcgagacgtctaatgaccattctccatatacctttcaacctaataagtaactgtgagatgtacagaaaggtgttcttacatgaagaagggtgtgaaggctgaacaatcatggatttttctgatcaattgtgctttaggaaattattgacagttttgcacaggttcttgaaaacgttatttataatgaaatcaactaaaactatttttgctataagttctataaggtgcataaaacccttaaattcatctagtagctgttccccc

SEQ ID NO: 96 - DNA, 442 bp, human
caccttgggtctgtgtcacttgtaggtttctctgcctccaggttgcctcaacagcaggaggcacagcagtttcaccatctttgaggtgagggtggggtgccccagctaggaagcaagatcgctgtgctaggtctgaccaaaaccagagggcagtctagtcctgggggtaaagccctcagatcccagggtacactcttctccattccctccacccacttgcctgtcaccccagtcacctaagcaatcactgggcccagaggagaggagacagacacacactggctcctggacctaaagggtatgagctggagctaaggccagctagagcttccactgtcagccctcactgtcagccccactgcacccccctgtgcctgctgggcactgggcactagctagatgctttaggttgcttcagctgatccttcaactctgtgaggtg

SEQ ID NO: 97 - DNA, 484 bp, human
tatgtgacgctggaccttttctttacccaaggatttttaaaactcagatttaaaacaaggggttactttacatcctactaagaagtttaagtaagtaagtttcattctaaaatcagaggtaaatagagtgcataaataattttgttttaatctttttgtttttcttttagacacattagctctggagtgagtctgtcataatatttgaacaaaaattgagagctttattgctgcattttaagcataattaatttggacattatttcgtgttgtgttctttataaccaccgagtattaaactgtaaatcataatgtaactgaagcataaacatcacatggcatgttttgtcattgttttcaggtactgagttcttacttgagtatcataatatattgtgttttaacaccaacactgtaacatttacgaattatttttttaaacttcagttttactgcattttcacaacatatcagacttcaccaa

SEQ ID NO: 98 - DNA, 509 bp, human
tgtgtaatacgccaaccagtcaagttgtgttttggccagagatttagatatgtccaatttcctggctcatttcattgtgctctatgggtacgtataaaaagcaagaattctgtttcctaggcaaacattgcaactcagggctaaagtcatccagtgaaacttttagagccagaagtaactttgtcccagtcctacaatgtgaaaagagtgaatagttgcctctttttagccattttcatggctggtacatattcgtacgcattacttttcagaatcaatacgcactttcagatattcttatttttattctcttaagtctttattaactttggagagagaaatgatgcatctttttattttaaatgaagtagatcaacatggtggaacaaaatgataaagaacagaaaacatttcaatatattactaataactttttccaatataaatcctaaaattcctataacatagtattttacagttttatgaagctttctattgtgacttttatg

SEQ ID NO: 99 - DNA, 453 bp, human
  misc_feature (250)..(250): n is a, c, g, or t
aacactcacatacaagttcatactttaaaagaggaaagctacttaacaatgacaaatatttcacaataataatttttacttatataccatctttcaactgaacatttcagttcttccaagagcttcttagagtagtatattttgggggcagtcaaggaataaactacagtgtaaacatatcccagatgaaaactgctgtatggaaaaatgacagaaagtaactgattgacactgttgattcacagttcangcctcctatctgggaaagacatttctttcctctgctcactttaagaacttttaccgactccaaaaatctcaggaattaaacttttaacagttacagcaataaagaatagttagtactccaaaaatattatatttaagatgctcaacaagaaaaaaatgcaaatgtaatatttttttcaaattacttctttattgacttgtcca

SEQ ID NO: 100 - DNA, 461 bp, human
gatttccaaatcagtcgttttctcaaaaaaatatcgtataagtgactcatcctgtctgctaactccagacctcccagcttgaagccaaatctttccatgtgagattgatatggatttcctagaagtactggaatgttgtcatatcttgccctattttaattctgctatagaaaacaattgccttcacttttaaggagtaatttgaatattaataactctggtctagattttcatataatgtattaaagacaaagtagtgaacatcaatgaacatctgatagagataaactgtaatcaggcataagcttgtttgtatgttctggcagtgactaatcagtaaatgatgtcggtttgcccagtatcacttatcttctgtatttttcctctgtcgtgtaaatagtataaccttttcatttatggacaattttttggactagtagccttcaatatacattctgctt

SEQ ID NO: 101 - DNA, 470 bp, human
acgcacacaactactacaattccgcctaggtccacaaggccttcactgttttccccccaagggaggctgctgctgctcttggctggagccagccagggccagccgaccccctctccctgcatggcatcctccagcccctgtagaactcaacctctacagggttagagtttggagagagcagactggcggggggcccattggggggaaggggaccctccgctctgtagtgctacagggtccaacatagaacagggtgtccccaacagcgcccaaaggacgcactgagcaacgctattccagctgtccccccactccctcacaagtgggtacccccaggaccagaagctcccccagcaaagcccccagagcccaggctcggcctgcccccaccccattcccgcagtgggagcaaactgcatgcccagagacccagcggacacacgcggtttggtttgcagcgactggcatac

SEQ ID NO: 102 - DNA, 476 bp, human
ccaaatttcatttcagccacttctgcaggatccctactgccaacctggaatggagacttttatctacttctctctctctgaagatgtcaaatcgtggtttagatcaaatatatttcaagctataaaagcaggaggttatctgtgcagggggctggcatcatgtatttaggggcaagtaataatggaatgctactaagatactccatattcttccccgaatcacacagacagtttctgacaggcgcaactcctccattttcctcccgcaggtgagaaccctgtggagatgagtcagtgccatgactgagaaggaaccgacccctagttgagagcaccttgcagttccccgagaactttctgattcacagtctcattttgacagcatgaaatgtcctcttgaagcatagctttttaaatatctttttccttctactcctccctctgactctaagaattctctcttctggaatcgcttg

SEQ ID NO: 103 - DNA, 501 bp, human
gtgtgatgcttttgtaggtacctggaaacttgtctccagtgaaaactttgatgattatatgaaagaagtaggagtgggctttgccaccaggaaagtggctggcatggccaaacctaacatgatcatcagtgtgaatggggatgtgatcaccattaaatctgaaagtacctttaaaaatactgagatttccttcatactgggccaggaatttgacgaagtcactgcagatgacaggaaagtcaagagcaccataaccttagatgggggtgtcctggtacatgtgcagaaatgggatggaaaatcaaccaccataaagagaaaacgagaggatgataaactggtggtggaatgcgtcatgaaaggcgtcacttccacgagagtttatgagagagcataagccaagggacgttgacctggactgaagttcgcattgaactctacaacattctgtgggatatattgttcaaaaagatattgttgttttccctgatttagcaagca

SEQ ID NO: 104 - DNA, 462 bp, human
ggtccccacagactcagagagaacccaccatggtgctgtctcctgccgacaagaccaacgtcaaggccgcctggggtaaggtcggcgcgcacgctggcgagtatggtgcggaggccctggagaggatgttcctgtccttccccaccaccaagacctacttcccgcacttcgacctgagccacggctctgcccaggttaagggccacggcaagaaggtggccgacgccctgaccaacgccgtggcgcacgtggacgacatgcccaacgcgctgtccgccctgagcgacctgcacgcgcacaagcttcgggtggacccggtcaacttcaagctcctaagccactgcctgctggtgaccctggccgcccacctccccgccgagttcacccctgcggtgcacgcctccctggacaagttcctggcttctgtgagcaccgtgctgacctccaaataccgttaagctg

SEQ ID NO: 105 - DNA, 520 bp, human
gcccaggctgacttacacattgaggatggagactccatccgcttcgggcgcttcgcgttggagaccagggccagccctggccacaccccaggctgtgtcaccttcgtcctgaatgaccacagcatggccttcactggagatgccctgttgatccgtgggtgtgggcggacagacttccagcaaggctgtgccaagaccttgtaccactcggtccatgaaaagatcttcacacttccaggagactgtctgatctaccctgctcacgattaccatgggttcacagtgtccaccgtggaggaggagaggactctgaaccctcggctcaccctcagctgtgaggagtttgtcaaaatcatgggcaacctgaacttgcctaaacctcagcagatagactttgctgttccagccaacatgcgctgtggggtgcagacacccactgcctgatctcacttctgtcagatgctcccatccactattaatgcactaggtgggaggagagggcggcaatgacactgcacct

SEQ ID NO: 106 - DNA, 515 bp, human
  misc_feature (89)..(89): n is a, c, g, or t
  misc_feature (110)..(110): n is a, c, g, or t
  misc_feature (227)..(227): n is a, c, g, or t
  misc_feature (250)..(250): n is a, c, g, or t
  misc_feature (276)..(276): n is a, c, g, or t
aattgccacattttcttatggcattaaaaattttacaaaaacataattttaatggctatattatattccatttaatggatgcaactcangtttatttaaccattcccatngttgttaactatttaggttgtttctaattttcattattataaagttgcagaaatttggtgtacataaaactgtctccatataattgattattaggatatattcccatgaaggattcntttttttaaaaaaatgtgaaatntcatcttgtacttacacctttcatgnaaagggatttcctgcttttgtactgcatgggtggcagttgtgaggaaaagccagtcaaatgacctttttacaaaagaaatgcagtggtcacttcagttgagagtgactttttaatacaacaagatcaactagaagaattcaactgtctcaagaatcaaggtaccccaatatatctcgcaattccaaactttgtttgagggactcgttatccagctcttggtagccacacctgcaatgta

SEQ ID NO: 107 - DNA, 532 bp, human
aagccttacagttatcctgcaagggacaggaaggtctgatttgcaggatttttagagcattaaaataactatcaggcagaagaatctttcttctcgcctaggatttcagccatgcgcgcgctctctctctttctctctcttttcctctctctccctctttctagcctggggcttgaatttgcatgtctaattcatttactcaccatatttgaattggcctgaacagatgtaaatcgggaaggatgggaaaaactgcagtcatcaacaatgattaatcagctgttgcaggcagtgtcttaaggagactggtaggaggaggcatggaaaccaaaaggccgtgtgtttagaagcctaattgtcacatcaagcatcattgtccccatgcaacaaccaccaccttatacatcacttcctgttttaagcagctctaaaacatagactgaagatttatttttaatatgttgactttatttctgagcaaagcatcggtcatgtgtgtattttttcatagtcccaccttggagcatttatg

SEQ ID NO: 108 - DNA, 415 bp, human
gaaggcggctgatgagagcgagagaggaatgaaggtcatcgaaaaccgggccatgaaggatgaggagaagatggaactgcaggagatgcagctgaaggaggccaagcacatcgctgaggattcagaccgcaaatatgaagaggtggccaggaagctggtgatcctggaaggagagctggagcgctcggaggagagggctgaggtggccgagagccgagccagacagctggaggaggaacttcgaaccatggaccaggccctcaagtccctgatggcctcagaggaggagtattccaccaaagaagataaatatgaagaggagatcaaactgttggaggagaagctgaaggaggctgagacccgagcagagtttgccgagaggtctgtggcaaagttggagaaaaccatcgatgac

SEQ ID NO: 109 - DNA, 366 bp, human
catggggggacttgaaccctgcagcaggctcctgctcctgcctctcctgctggctgtaagtggtctccgtcctgtccaggcccaggcccagagcgattgcagttgctctacggtgagcccgggcgtgctggcagggatcgtgatgggagacctggtgctgacagtgctcattgccctggccgtgtacttcctgggccggctggtccctcgggggcgaggggctgcggaggcagcgacccggaaacagcgtatcactgagaccgagtcgccttatcaggagctccagggtcagaggtcggatgtctacagcgacctcaacacacagaggccgtattacaaatgagcccgaatcatgacagtcagcaa

SEQ ID NO: 110 - DNA, 480 bp, human
tccccagcagtggctcggtgagccatgtgcacctatggcccagccactgcagcacaggaggctccgtgagccttggttcctccccgaaaacccccagcattacgatcccccaagtgtcctggaccctggcctaaagaatcccacccccacttcatgcccactgccgatgcccaatccaggcccggtgaggccaaggtttcccagtgagcctctgcgcctctccactgtttcatgagcccaaacaccctcctggcacaacgctctaccctgcagcttggagaactccgctggatgggagtctcatgcaagacttcactgcagcctttcacaggactctgcagatagtgcctctgcaaactaaggagtgactaggtgggttggggaccccctcaggattgtttctcggcaccagtgcctcagtgctgcaattgagggctaaatcccaagtgtctcttgactggctcaagaattagggcccca

SEQ ID NO: 111 - DNA, 475 bp, human
cagaccggcagtcatcatggcagtttcagcgttcaaacagcaatagctcaagtgtgataactactgaggataataaaatccacattcacttaggaagtccttacatgcaagctgtagccagcccttcagcaccactgcaggataaccgaactcaaggcttaattaacggggcactaaacaaaacaaccaataaagtcaccagcagtattactatcacaccaacagccacacctcttcctcgacaatcacaaattacagtaagtaatatatataactgaccacgctcaccctcatccagtccatactgatatttttgcaaggaactcaatccttttttaatcatccctccatatcccccaagactgactgaactcgtactttgggaaggtttgtgcatgaactatacaagagtatctgaaactaactgttgcctgcatagtcatatcgagtgtgcacttactgtatatcttttcat

SEQ ID NO: 112 - DNA, 344 bp, human
  misc_feature (173)..(173): n is a, c, g, or t
gcgtgttttcctcttgatcgggaactcctgcttctccttgcctcgaaatggaccccaactgctcctgctcgcctgttggctcctgtgcctgtgccggctcctgcaaatgcaaagagtgcaaatgcacctcctgcaagaagagctgctgctcctgctgccctgtgggctgtgcnaagtgtgcccagggctgcatctgcaaagggacgtcagacaagtgcagctgctgtgcctgatgccaggacagctgtgctctcagatgtaaatagagcaacctatataaacctggatttttttttttttttttttgtacaaccctgacccgtttgctacatctttttttctat

SEQ ID NO: 113 - DNA, 318 bp, human
ttccacatcagtaactgccctggggtttgtgctgtacaaatacaagctcctgccacggtcttgaagttctgttcttatgctctctgctcactggttttcaataccaccaagaggaaaatattgacaagtttaaaggctgtgtcattgggccatgtttaagtgtactggatttaactacctttggcttaattccaatcattgttaaagtaaaaacaattcaaagaatcacctaattaatttcagtaagatcaagctccatcttatttgtcagtgtagatcaactcatgttaattgatagaataaagccttgtgatcact

SEQ ID NO: 114 - DNA, 411 bp, human
ctttgcgggcacagagactgccacaaagtggagcggctacatggaaggggcagttgaggctggagaacgagcagctagggaggtcttaaatggtctcgggaaggtgaccgagaaagacatctgggtacaagaacctgaatcaaaggacgttccagcggtagaaatcacccacaccttctgggaaaggaacctgccctctgtttctggcctgctgaagatcattggattttccacatcagtaactgccctggggtttgtgctgtacaaatacaagctcctgccacggtcttgaagttctgttcttatgctctctgctcactggttttcaataccaccaagaggaaaatattgacaagtttaaaggctgtgtcattgggccatgtttaagtgtactggatttaactacctttg

SEQ ID NO: 115 - DNA, 454 bp, human
taattgcttgttttctagcctggcaagatattttcataaaagagggataacaatgctgattactaccttttaaaatattttagataaatgcacagcaccacagcaccacatctaagcattagtgatgggtagctgatgtcagcttcatgtggattttaagcactctagaaacaatgaagcttcttggcatattttaaggagctcccaaaatgtgttacctattaaattgtaactcagcaagtagaagaccatttgaaaagtcaggtacaaatttcctcaagtggcataaaaatgtagtcagttttctcttttaccagtttttatttccactccaattatttagaactttatttgtacatgtgcagaagaataaggcagctgagaatcttgtttcccccaagagagttttacaggctgagtgttgcaaatgtgttctttgtcctgttatatgtat

SEQ ID NO: 116 - DNA, 482 bp, human
gcactgccatgtatgaaagtctctttatgatgtttgtttttttgtcatttttgttctttacatcaagaaattttatgtttaaatatgcggagaatgtatattgcctctgctcctatcagggttgctaaaccctggtacatcgtatataaaatgtattaaaactggggtttgttaccagttgctgtactttgtatatagaatttttataaattgtatgcttcagaaataatttatttttaaaaagaaattaaaagttttaaactcacatccatattacacctttcccccctgaaatgtatagaatccatttgtcatcaggaatcaaaacccacagtccattgtgaagtgtgctatatttagaacagtcttaaaatgtacagtgtattttatagaattgaagttaacattcttattttcaagagaatttatggacgttgtagaaatgtacaaatgcatttccaaactgccttaaacgttgtatt

SEQ ID NO: 117 - DNA, 422 bp, human
gggactgtctgtcaaaagactctgtatatcttttgtggatgagttttgtgagagaacagagagaccattgtacctggcacaagggctcttcatgaaaagggagacttactgggaggtgcaagacagtggcatttctcctctcctcttgctgctcagcacagccctggattgcagccccgaggctgagaccagacaaagcccgggaggcagaaagatgctccaagaaccaacactatcaatgtctttgcaaatcctcacaggattcctgtgggtccagctttggaactgggaaacctttcttcggatccgcactcattccactgatgccagctgcccctgaaggatgccagtactgtggtgtgtgagtctcagcagccgcccacacgctcctaactctgctgcatggcagatgcctaggtgga

SEQ ID NO: 118 - DNA, 357 bp, human
attaatcagccccagagtgctttaaaaaattctcttaaataaaaataatagactcgctagtcagtaaagatatttgaatatgtatcgtgccccctccggtgtctttgatcaggatgacatgtgccatttttcagaggacgtgcagacaggctggcattctagattacttttcttactctgaaacatggcctgtttgggagtgcgggattcaaaggtggtcccaccgctgcccctactgcaaatggcagttttaatcttatcttttggcttctgcagatggttgcaattgatccttaaccaataatggtcagtcctcatctctgtcctgcttcataggtgccaccttgtgtgtttaaa

SEQ ID NO: 119 - DNA, 328 bp, human
ctacgcgtgtccttgggcggagaagggaggtgactccggcggaagaggacaaggcagaatgcaggcccttcgggtgtcccaggcgctgatccgctccttcagctccaccgcccggaaccgctttcagaaccgagtgcgcgagaaacagaagctcttccaggaggacaatgacatcccgttgtacctgaagggcggcatcgttgacaacatcctgtaccgagtgacaatgacgctgtgtctgggcggcactgtctacagcttgtactcccttggctgggcctccttccccaggaattaagaccaagaagcctggggggcctgagagact

SEQ ID NO: 120 - DNA, 417 bp, human
tggtgacacaaacagccttttcccaggtacttggtacctggagcgagtggacgagcagcatcgccgaaagtatgcccggcgtcccgtctaaaggtgttctgcagatccatggaaagcttcctgggaaacgtatgctagcagagcttctccccgtgaatcatatttttaagatcccactcttagctggtaaatgaatttgaatcgacatagtagccccataagcatcagccctgtagagtgaggagccatctctagcgggcccttcattcctctccatgctgcaatcactgtcctgggcttatggtgcctatggactaggggtcctttgtgaaagagcaagatggagcaatggagagaagacctcttcctgaatcactggactccagaaatgtgcatgcagatcagctgttgccttca

SEQ ID NO: 121 - DNA, 533 bp, human
ttgatgccagcatttgcatcccgggctccatcacattcatgcccaatggatgctgcaagacctgcacccctcgcaatgagaccagggtgccctgctccaccgtccccgtcaccacggaggtttcgtacgccggctgcaccaagaccgtcctcatgaatcattgctccgggtcctgcgggacatttgtcatgtactcggccaaggcccaggccctggaccacagctgctcctgctgcaaagaggagaaaaccagccagcgtgaggtggtcctgagctgccccaatggcggctcgctgacacacacctacacccacatcgagagctgccagtgccaggacaccgtctgcgggctccccaccggcacctcccgccgggcccggcgctcccctaggcatctggggagcgggtgagcggggtgggcacagcccccttcactgccctcgacagctttacctcccccggaccctctgagcctcctaagctcggcttcctctcttcagatatttattgtctgagtctttgttcagtccttg

SEQ ID NO: 122 - DNA, 417 bp, human
cttgcttatatcatgtgctgccgacgggaaggcgtggaaaagagaaacatgcaaacaccagacatccaactggtccatcacagtgctattcagaaatctaccaaggagcttcgagacatgtccaagaatagagagatagcatggcccctgtcaacgcttcctgtgttccaccctgtgactggggaaatcatacctcctttacacacagacaactatgatagcacaaacatgccattgatgcaaacgcagcagaacttgccacatcagactcagattccccaacagcagactacaggtaaatggtatccctgaagaaagaaaactgactgaagcaatgaatttataatcagacaatatagcagttacatcacatttcttttctcttccaataatgcatgagcttttctggcatatgtt

SEQ ID NO: 123 - DNA, 542 bp, human
cctcccgggaggacagccttgaggcgggcctgcccctccaggtccgaggctaccccgaggagaagaaagaggaggagggcagcgcaaaccgcagaccagaggaccaggagctggagagcctgtcggccattgaagcagagctggagaaagtggcccaccagctgcaggcactacggcggggctgagacaccggctggcagggctggccccagggcaccctgtggccctggctctgctgtccccttggcaggtcctggccagatggcccggacgctgcttccggtagggaggcagcctccagcctgcccaagcccaggccaccctatcgccccctacgcgccttgtctcctactcctgactcctacctgccctggaacatcctttgcagggcagccccacaactttaaacattgacgattccttctctgaacacaggcagctttctagaagtttcccttcctccatcctatccactgggcacaactgcaataacttctgaccttttggtgaaagctgagaactcctgactgtaacatattctg

SEQ ID NO: 124 - DNA, 537 bp, human
gcaggccatactggttccattgttctgtataatactgaataaataaatttacttttacatgatcgtataagtttctagataagataaacaaattctgtttaaatttttttaataaaaatcttaaaacactttttttctaacctagactgagaaattcatgtttacttttctaggtgtatgatactttgtaaagttgatactttcctaagaatttaacatgtcatatttttgaaatagatttaagtgtgcttcttattgctaaaaatactaaatgtcatgggtcatagtatctgatatcaatatcgttgataacatatccacaggtaacaccatgatgtaggcataaatggaaaacaaaaaccctactatttcaaatatattgtacttttttatttctgtaagccaactgtgtgccattttcactggacttttaaatctagactttagtgatgtctacattgtaaatgatcttttgtggatatttgtcacttggtttcagaaagttcacaaatgtagcaacagctcacatgactgagt

SEQ ID NO: 125 - DNA, 312 bp, human
tcccttctcgcttgggaactctagtctcgcctcgggttgcaatggaccccaactgctcctgtgccgctggtgtctcctgcacctgcgccagctcctgcaagtgcaaagagtgcaaatgcacctcctgcaagaagagctgctgctcctgctgccctgtgggctgtgccaagtgtgcccaaggctgcatctgcaaaggggcatcggagaagtgcagctgctgcgcctgatgtcgggacagccctgctcccaagtacaaatagagtgacccgtaaaatctaggattttttgttttttgctacaatcttgacccct

SEQ ID NO: 126 - DNA, 393 bp, human
aaaggctggcatcttatggctcatcaaaggcggctgtgaccatgttctcatcagttatgagactggagctttccaagtggggaattaaagttgcttccatccaacctggaggcttcctaacaaatatcgcaggcaccagtgacaagtgggaaaagctggagaaggacattctggaccacctccccgctgaggtacaggaagactacggccaggactacatcttagcacagcggaatttcctcctattgatcaactcgttagccagcaaggacttctctccggtgctgcgggacatccagcatgctatcttggcgaagagcccttttgcctattacacgccagggaaaggcgcttacttgtggatctgccttgctcactatttgcctattggca

SEQ ID NO: 127 - DNA, 460 bp, human
ggtgtcgaactatatgccttgtatgatcagttttatgtggctaatgagtttctcaaatatcgtttacacgttggtaactataatggcacagctggagatgcattacgtttcaacaaacattacaaccacgatctgaagtttttcaccactccagataaagacaatgatcgatatccttctgggaactgtgggctgtactacagttcaggctggtggtttgatgcatgtctttctgcaaacttaaatggcaaatattatcaccaaaaatacagaggtgtccgtaatgggattttctggggtacctggcctggtgtaagtgaggcacaccctggtggctacaagtcctccttcaaagaggctaagatgatgatcagacccaagcactttaagccataaatcactctgttcattcctccaggtattcgttatctaatagggcaattaattccttcagcacttt

SEQ ID NO: 128 - DNA, 525 bp, human
ccaggaggggctgtccaatcacattcaggcatgcgaatgagctgggccctgggtgaggtgggggtctggcctagtggggaggggcctggcctgggtggggcagggcctggcctggtccaggcttgggctccattcccatcactgctgtccctcctgaggtctggattggggatggggacaaagaaatagcaagagatgagaaacaacagaaacttttttctctaaaggactggttaaatcaattctgatacagccttacaatacaatagtatgcagctaaaaaataattgtatgtctttatatactaatatgtaataatcttcaggtgaaaaaggcaagccacagaaatgtgtatagcgcacttcccatttgtgtttcagaaaggagtagaatataaacacataattgcttatgtatgcctattcagaataaatgggtaacactgattacttttgggaggggaaccagtaggttgaggacaggagagggaagggtcttaacacttacacccttttgtacattttg

SEQ ID NO: 129 - DNA, 515 bp, human
aggagcccaggaacgacgtggtcttccagcccatttccggggaagacgtgcgcgatgtgacagccctgaacgtgagcacgctgaaggcttacttcagatgcgatggctacaagggctacgacctggtctacagcccccagagcggcttcacctgcgtgtccccgtgcagtaggggctactgtgaccatggaggccagtgccagcacctgcccagtgggccccgctgcagctgtgtgtccttctccatctacacggcctggggcgagcactgtgagcacctgagcatgaaactcgacgcgttcttcggcatcttctttggggccctgggcggcctcttgctgctgggggtcgggacgttcgtggtcctgcgcttctggggttgctccggggccaggttctcctatttcctgaactcagctgaggccttgccttgaaggggcagctgtggcctaggctacctcaagactcacctcatccttaccgcacatttaaggcgccattgcttttgggagact

SEQ ID NO: 130 - DNA, 339 bp, human
agcagcttattgtttctctgaaagtgtgtgtagttttactttcctaaggaattaccaagaatatcctttaaaatttaaaaggatggcaagttgcatcagaaagctttattttgagatgtaaaaagattcccaaacgtggttacattagccattcatgtatgtcagaagtgcagaattggggcacttaatggtcaccttgtaacagttttgtgtaactcccagtgatgctgtacacatatttgaagggtctttctcaaagaaatattaagcatgttttgttgctcagtgtttttgtgaattgcttggttgtaattaaattctgagcctgatattgatatg

SEQ ID NO: 131 - DNA, 377 bp, human
gagaacgggtacattcacccggtcaacctgacgtggccctttatggtggccgggaaacccgagagtgacctgaaagaagtggtgaccgcgagccgcttatgtggaaccaccgcgtcctgaccttggaggtgcgagtctgggaaaggcgcgctcccggggggagcgggccccgggaaggcgacccctgccctcagtgctctctgtctctgcttccccctcgcaatgctcctctctctgtcccaccccgcgagaacactttacaacgacgaggagattcgtttccaaaccagaggagatcaattgtacttacaaagattcccatctatttaactttattaacttctaccgtgaatgactctgcaagccttgctggtcca

SEQ ID NO: 132 - DNA, 252 bp, human
ttctctcgaccacttaaaacttcagacttcctgtcctgctggtatcatggagaaagtccaatacctcactcgctcagctataagaagagcctcaaccattgaaatgcctcaacaagcacgtcaaaagctacagaatctatttatcaatttctgtctcatcttaatatgtctcttgctgatctgtatcatcgtgatgcttctctgaagttctgctacaacctctagatctgcagcttgccacatcagcttaaa

SEQ ID NO: 133 - DNA, 191 bp, human
tggcccatctattacatctacagctgacccttgaacatgggggttaggggagctgacaattcgtgggtccgcaaaatcttaactacctaatagcctactattgaccataaaccttactgataacataaacagtaaattaacacatattttgcgtgttatatgtattatacactatattcctacaataaagt

SEQ ID NO: 134 - DNA, 371 bp, human
aaatctgttctaagacatatgatcaacagatgagaactggtggttaatatgtgacagtgagattagtcatatcactaatatactaacaacagaatctaatcttcatttaaggcactgtagtgaattatctgagctagagttacctagcttaccatactatatctttggaatcatgaaaccttaagacttcagaatgattttgcaggttgtcttccattccagcctaacatccaatgcaggcaaggaaaataaaagatttccagtgacagaaaaatatattatctcaagtattttttaaaaatatatgaattctctctccaaatattaactaattattagattatattttgaaatgaacttgttggcccatc

SEQ ID NO: 135 - DNA, 441 bp, human
ggacaaagagcgctatgtctccctggtgatgcctgtggccctgttcaacctgattgacacttttcccttgagaaaagaagagatggtcctacaagccgaaatgagccagacctgtaacacctgacatgatggttcctctcttggcaattcctcttcattgtctacatagtgacatgcacacgggaaagccttaaaaatatccttgatgtacagattttatttgtaattttaaaagtctattttattatgagctttctttgcacttaaaaattagcatgctgctttttgtacttggaagtgtttcaaaaaattatatgaccatatttactctttctaacctttctttactccatcatggctggttgatttgtagagaaattagaacccataaccatacacaggctatcaacatgttattcaatgtgacacctaactcttttc

SEQ ID NO: 136 - DNA, 402 bp, human
  misc_feature (353)..(353): n is a, c, g, or t
tactcatgcctttttgtttaggataaataggtaagcacaaagagctcttcaaaatcagaaaaaacaataggagtccttccttgtcttttctgtgatctctgtccttgtttctgagactttctctaccattaagctctattttagctttcagttattctagtttgtttcccatggaatctgtcctaaactggtgtttttgtcagtgacagtcttgccagtcagcaatttctaacagcattttaaatgagtttgatgtacagtaaatattgatgacaatgacagcttttaactcttcaagtcacctaaagctattatgcaggaggatttagaagtcacattcataaaacccaagngctatgggtgtattattcatgatagctggcccacaggtcatgaattgag

SEQ ID NO: 137 - DNA, 174 bp, human
agaagctcaccaagtcaactaaacagccccgaggacttacatcaccttctcagctcttgacctcagaaagtatccaaaccaaggaggagaaacctgtgggtggcttgtcctccagtgacacaatggattaccgacagtgactaagggcagcatgtttaacccaggtgaagatct

SEQ ID NO: 138 - DNA, 371 bp, human
tttctggccttcacccagacgaagaccttccacgaggccagcgaggactgcatctcgcgcgggggcaccctgagcacccctcagactggctcggagaacgacgccctgtatgagtacctgcgccagagcgtgggcaacgaggccgagatctggctgggcctcaacgacatggcggccgagggcacctgggtggacatgaccggcgcccgcatcgcctacaagaactgggagactgagatcaccgcgcaacccgatggcggcaagaccgagaactgcgcggtcctgtcaggcgcggccaacggcaagtggttcgacaagcgctgccgcgatcagctgccctacatctgccagttcgggatcgtgtagccggc

SEQ ID NO: 139 - DNA, 406 bp, human
gcagactgggagttgctagcaaacaaatggcttacttacaaaagcagcttttagttcagacttagtttttataaaatgagaattctgacttacttaaccaggtttgggatggagatggtctgcatcagctttttgtattaacaaagttactggctctttgtgtgtctccaggtaactttgcttgattaaacagcaaagccatattctaaattcactgttgaatgcctgtcccagtccaaattgtctgtctgctcttatttttgtaccatattgctcttaaaaatcttggtttggtacagttcataattcaccaaaaagttcatataatttaaagaaacactaaattagtttaaaatgaagcaatttatatctttatgcaaaaacatatgtctgtctttgcaaagga

SEQ ID NO: 140 - DNA, 388 bp, human
gcacccagccttttaccatgtgggtttctttagtgtcttaaaagcgtccataagccaccattctgtggaaccaaggccccctccacgcaaacaccctccctcctggggacctctggagcctcagccagaagtaccattaggtttaattttaatttgttttgctggagaaacatcaggtttgtaggagactgagttgttagcaggtgtgcttagctcttgatagtgaacgtgtaccttgggaactggctcacccacctgctaatagcaccatcgtcactattaagcagacatttcagttggtagaatccatgtagaagtcatggacttttctgggaaatgacttttctgggaaatgacagtttctttgacatattttctttgcccactt

SEQ ID NO: 141 - DNA, 317 bp, human
cgccatcaccgagcgcttgatgtgcgcggagagcaatcgccgggacagctgcaagggtgactccgggggcccgctggtgtgcgggggcgtgctcgagggcgtggtcacctcgggctcgcgcgtttgcggcaaccgcaagaagcccgggatctacacccgcgtggcgagctatgcggcctggatcgacagcgtcctggcctagggtgccggggcctgaaggtcagggtcacccaagcaacaaagtcccgagcaatgaagtcatccactcctgcatctggttggtctttattgagcacctactatatgcagaaggggag

SEQ ID NO: 142 - DNA, 317 bp, human
gggccacgccaggaatattcagaaaataatgagaactacattgaagtgccattgatttttgatcctgtcacaagagaggatttgcacatggattttaaatgtgttgtccataataccctgagttttcagacactacgcaccacagtcaaggaagcctcctccacgttctcctggggcattgtgctggccccactttcactggccttcttggttttggggggaatatggatgcacagacggtgcaaacacagaactggaaaagcagatggtctgactgtgctatggcctcatcatcaagactttcaatcctatcccaa

SEQ ID NO: 143 - DNA, 456 bp, human
gctgctctggttctcatgacggcagatgcagcgaagaggctcaatgttacaccactggcaagaatagtagcatttgctgacgctgctgtagaacctattgattttccaattgctcctgtatatgctgcatctatggttcttaaagatgtgggattgaaaaaagaagatattgcaatgtgggaagtaaatgaagcctttagtctggttgtactagcaaacattaaaatgttggagattgatccccaaaaagtgaatatcaatggaggagctgtttctctgggacatccaattgggatgtctggagccaggattgttggtcatttgactcatgccttgaagcaaggagaatacggtcttgccagtatttgcaatggaggaggaggtgcttctgccatgctaattcagaagctgtagacaacctctgctatttaaggagacaaccctatgtgaccagaa

SEQ ID NO: 144 - DNA, 507 bp, human
ggaaagcaggattccatcgctggaacaattacatgatggactggaaaaatcaatttaacgattacactagcaagaaagaaagttgtgtgggtctctaattaatagatttaccctttatagaacatattttcctttagatcaaggcaaaaatatcaggagcttttttacacacctactaaaaaagttattatgtagctgaaacaaaaatgccagaaggataatattgattcctcacatctttaacttagtattttacctagcatttcaaaacccaaatggctagaacatgtttaattaaatttcacaatataaagttctacagttaattatgtgcatattaaaacaatggcctggttcaatttctttctttccttaataaatttaagttttttccccccaaaattatcagtgctctgcttttagtcacgtgtattttcattaccactcgtaaaaaggtatcttttttaaatgaattaaatattgaaacactgtacaccatagtttaca

SEQ ID NO: 145 - DNA, 489 bp, human
gagggtgatgccatctaaccctgcccctgtccaccccgggtgggtgaaactcactgagcagccaagactgttgcccgaggactcactgtatggtgccctctccaaagggtcgggagggtagctctccaggccagagcttgtgtccttcaacagagaggccagcggcaactggtccgttactggccaagggctctgaagaatcaacggtgctggtacaggatacaggaataaattgtatcttcacctggttcctaccctcgtccctacctgtcctgatcctggtcctgaagacccctcggaacaccctctcctggtggcaggccacttccctcccagtgccagtctccatccaccccagagaggaacaggcgggtgggccatgtggttttctccttcctggccttggctggcctctggggcaggggtggtggagagatggaagggcatcaggtgtagggaccctgccaagtggcacctgatttactctag

SEQ ID NO: 146 - DNA, 480 bp, human
ttaatgtgccaaggagccgttttctgcctgtcaaaaccacatcagatctcttgctggtgatgtcaaacctctatagtcttaatgcaggatctctgacaatgagtgaaaagcgggaatttcctacagtgcccttggttaaattaggcagttcttttacgaaggttcaagattatctaagaagatttgaaagtataccagatatgcttgaattggatcacctcacagtttcaggagatgtgacatttggaaaaaatgtttcattaaagggaacggttatcatcattgcaaatcatggtgacagaattgatatcccacctggagcagtattagagaacaagatagtgtctggaaaccttcgcatcttggaccactgaaatgaaaaatactgtggacacttaaataatgggctagtttcttacaatgaaatgttctctaggatttaggcactaaaaggtactttactatgttactgtaccctgc

SEQ ID NO: 147 - DNA, 529 bp, human
caagaatgatgggcactaccgtggagatcccaactggtttatgaagaaagcgcaggagcataagagggaattcacagagagccagctgcaggagggaaagcatgtcattggccttcagatgggcagcaacagaggggcctcccaggccggcatgacaggctacggacgacctcggcagatcatcagttagagcggagagggctagccctgagcccggcgctcccccagctccttggctgcagccatcccgcttagcctgcctcacccacacccgtgtggtaccttcagccctggccaagctttgaggctctgtcactgagcaatggtaactgcacctgggcagctcctccctgtgcccccagcctcagcccaacttcttacccgaaagcatcactgccttggcccctccctcccggcggcccccatcacctctactgtctcctccctgggctaagcaggggagaagcgggctgggggtagcctggatgtgggcgaagtccactgtcctccttggcggcaaaagcccatt

SEQ ID NO: 148 - DNA, 525 bp, human
tcaaagacttcgtgattatccccctgcacaccaccccagagacatccgttaaggagatcgatgagttggttgaggtctacacggacgtgaaacaccgctggaaggcggagaatttcattttcatgggtgacttcaatgccggctgcagctacgtccccaagaaggcctggaagaacatccgcttgaggactgaccccaggtttgtttggctgatcggggaccaagaggacaccacggtgaagaagagcaccaactgtgcatatgacaggattgtgcttagaggacaagaaatcgtcagttctgttgttcccaagtcaaacagtgtttttgacttccagaaagcttacaagctgactgaagaggaggccctggatgtcagcgaccactttccagttgaatttaaactacagtcttcaagggccttcaccaacagcaaaaaatctgtcactctaaggaagaaaacaaagagcaaacgctcctagacccaagggtctcatcttattaaccatttcttgcctctaaata

SEQ ID NO: 149 - DNA, 559 bp, human
aacgaggagcacatgaccctgctgaagatgattttgataaaatgctgtgatatctctaacgaggtccgtccaatggaagtcgcagagccttgggtggactgtttattagaggaatattttatgcagagcgaccgtgagaagtcagaaggccttcctgtggcaccgttcatggaccgagacaaagtgaccaaggccacagcccagattgggttcatcaagtttgtcctgatcccaatgtttgaaacagtgaccaagctcttccccatggttgaggagatcatgctgcagccactttgggaatcccgagatcgctacgaggagctgaagcggatagatgacgccatgaaagagttacagaagaagactgacagcttgacgtctggggccaccgagaagtccagagagagaagcagagatgtgaaaaacagtgaaggagactgtgcctgaggaaagcggggggcgtggctgcagttctggacgggctggccgagctgcgcgggatccttgtgcagggaagagctgccctgggcacctggcaccacaagaccatgttttctaa

SEQ ID NO: 150 - DNA, 542 bp, human
tgacgcaaaataccaccttggcgcctacacgggagacgacgtccgcatcgtccgtgacgacatgctgtgtgccgggaacacccggagggactcatgccagggcgactccggagggcccctggtgtgcaaggtgaatggcacctggctgcaggcgggcgtggtcagctggggcgagggctgtgcccagcccaaccggcctggcatctacacccgtgtcacctactacttggactggatccaccactatgtccccaaaaagccgtgagtcaggcctgggttggccacctgggtcactggaggaccaacccctgctgtccaaaacaccactgcttcctacccaggtggcgactgccccccacaccttccctgccccgtcctgagtgccccttcctgtcctaagccccctgctctcttctgagccccttcccctgtcctgaggacccttccctatcctgagcccccttccctgtcctaagcctgacgcctgcaccgggccctccagccctcccctgcccagatagctggtggtgggcgctaatcct

SEQ ID NO: 151 - DNA, 220 bp, human
gaagagggagctctattgccaccatgagtttctccggcaagtaccaactgcagagccaggaaaactttgaagccttcatgaaggcaatcggtctgccggaagagctcatccagaaggggaaggatatcaagggggtgtcggaaatcgtgcagaatgggaagcacttcaagttcaccatcaccgctgggtccaaagtgatccaaaacgaattcacggtggg

SEQ ID NO: 152 - DNA, 524 bp, human
ccttctctggtctccttgagatgatcgtagacacagggatgattcccacccaaacccacgtattcattcagtgagttaaacacgaattgatttaaagtgaacacacacaagggagcttgcttgcagatggtctgagttcttgtgtcctggtaattcctctccaggccagaataattggcatgtctcctcaacccacatggggttcctggttgttcctgcatcccgatacctcagccctggccctgcccagcccatttgggctctggttttctggtggggctgtcctgctgccctcccacagcctccttctgtttgtcgagcatttcttctactcttgagagctcaggcagcgttagggctgcttaggtctcatggaccagtggctggtctcacccaactgcagtttactattgctatcttttctggatgatcagaaaaataattccataaatctattgtctacttgcgattttttaaaaaatgtatatttttatatatattgttaaatcctttgcttcattcca

SEQ ID NO: 153 - DNA, 486 bp, human
tctcccaacctctactgtaaactttctggtccgagaacgagccgaacacagcgcgacgcagggactaggacggcccggtgaccgcgcggattcaggattgcggggacgcagaaaggttaaggcacttttaaaaactatagcaaggctcctgtttatttattctactttctttccctaataatcaaaacaccgcgtaggctcctccgtttatcagtattaatggtgtaactttgttggcaatatttgccgtgtagaattttttttagatatccattgtaaatttgaaacaaagaccgatctgtgtaaaaacaaatttccatatgttttatataaatatatatataatatgaaggactaccctcctttttttttttttgtattttggctgctagagtgcagcatttgtgacacgtatttgaaatttgaaatttccttctgcactgtataaaaggaccatttgaggatgttttgccttttgtgtatttt

SEQ ID NO: 154 - DNA, 560 bp, human
gagccccattcacaaattttgacccctctactctccttccttcatccctggatttctggacctaccctggctctctgactcatcctcctctttatgagagtgtaacttggatcatctgtaaggagagcatcagtgtcagctcagagcagctggcacaattccgcagccttctatcaaatgttgaaggtgataacgctgtccccatgcagcacaacaaccgcccaacccaacctctgaagggcagaacagtgagagcttcattttgatgattctgagaagaaacttgtccttcctcaagaacacagccctgcttctgacataatccagttaaaataataatttttaagaaataaatttatttcaatattagcaagacagcatgccttcaaatcaatctgtaaaactaagaaacttaaattttagttcttactgcttaattcaaataataattagtaagctagcaaatagtaatctgtaagcataagcttatcttaaattcaagtttagtttgaggaattctttaaaattacaactaagtgatttgtatgtctatttttttc

SEQ ID NO: 155 - DNA, 521 bp, human
ggcagcttttttatcagccttgctttggataggacctccaaggactaagcctccagccccatgtgtgacccttgtcatctctctgccccacataattatgttactttgctatgtgctcctaatgtatctagtgtgtcctgtgacaacactcatcacacttcattgtaaatcacttgttttattgactgtctttcctatagactgtaagctccatgagggcaggcacatgttgttctcattgaccgtgctggccccagtgcctagatgcatggctggcacattgttggcactcaacaatggttgaatgaataaaacaataaatgaatgaataactaagatatagaaactctcatttatattgcagattgaatatatatgatgaaattcttatgttgaatatgttagaatcaaatactcatttttcattagatacagtagtgtcatcactcttttaagatcttgttaaagatttcaaataaaggtacttctggcgagccaggctgcacagcatttgctttcct

SEQ ID NO: 156 - DNA, 268 bp, human
tatttgaatatgtatcgtgccccctccggagtctttgatcaggatgacatgtgccatttttcagaggacgtgcagacaggctggcattctagattacttttcttactctgaaacatggcctgtttgggagtgcgggattcaaaggtggtcccacggctgcccctactgcaaatggcagttttaatcttatcttttggcttctgcagatggttgcattgatccttaaccaataatggtcagtcctcatctctgtcctgacttcataggt

SEQ ID NO: 157 - DNA, 529 bp, human
ctttctatattgttatcagtccaggaaacaggtaaacagatgtaattagagacattggctctttgtttaggcctaatctttctttttacttttttttttcttttttctttttttttaaagatcatgaatttgtgacttagttctgccctttggagaacaaaagaaagcagtcttccatcaaatcaccttaaaatgcacggctaaactattcagagttaacactccagaattgttaaattacaagtactatgctttaatgcttctttcatcttactagtatggcctataaaaaaaataataccacttgatgggtgaaggctttggcaatagaaagaagaatagaattcaggttttatgttattcctctgtgttcacttcgccttgctcttgaaagtgcagtatttttctacatcatgtcgagaatgattcaatgtaaatatttttcattttatcatgtatatcctatacacacatctccttcatcatcatatatgaagtttattttgagaagtctacattgcttacattt

SEQ ID NO: 158 - DNA, 494 bp, human
taacaatccatgatgctgttttgcatattttgatgaagaaagattacagtacttcaaagtttaatcccagtcaggaaaaagatggaaaaattgattttaccataaatacaaatggaggattacgtaatcgggtatatgaggtgccagttgaaacaaaattctaatcaacatataattcagaaggatcttcatctgactatgacataaaaacaactttatacccagaaagttattgataagttcatacattgtacgaagagtatttttgacagaatatgtttcaaactttggaacaagatggttctagcatggcatatttttcacatatctagtatgaaattatataagtattctaaattttatatcttgtagctttatcaaagggtgaaaattattttgttcatacatatttttgtagcactgacagatttccatcctagtcactaccttcatgcataggtttagcagtatagtggcgccactgttttgaatct

SEQ ID NO: 159 - DNA, 469 bp, human
tcatcaggctccagttattctccatctcccagctcagctttttctgtctgtaagcctgattttcaggaaggctctttcctagtgatggagatgaccaccatcagctccaggcttctatcctgctaacccagtaacccagtgggaagagatttacttattccaataattccaagtggagagtgtcattgacccgtttggggtctcatctctacttctaggggaatgaaacactttgagtggccaggcctgtgtcatgtgctaattcctagagccagggaaataaggtctgaggattcaggatggggtgaaaggtggttgcttaaaggaaaatgaaatacaattagcagaataaggggaaacgagtggtctgctctgctcgggcaaaacaagagatgcccattactgtgagggacccttgaagtctggactcttaaatgggtttttgctgatttcctgggtgcatgctagg

SEQ ID NO: 160 - DNA, 269 bp, human
accagcaacaacttcaatccggtggagaacaaagatattgtggttttaacctgtcaacctgagactcagaacacaacctacctgtggtgggtaaacaatcagagcctcctggtcagtcccaggctgctgctctccactgacaacaggaccctcgttctactcagcgccacaaagaatgacataggaccctatgaatgtgaaatacagaacccagtgggtgccagccgcagtgacccagtcaccctgaatgtccgctatgagtcagtaca

SEQ ID NO: 161 - DNA, 513 bp, human
atattagttaccctggtgtgctgtattctctaaaacctttaaatgtttgcatgcagccattcgtcaaatgtcaaatattctctctttggctggaatgacaaaaactcaaataaatgtatgattaggaggacatcataacctatgaatgatggaagtccaaaatgatggtaactgacagtagtgttaatgccttatgtttagtcaaactctcatttaggtgacagcctggtgactccagaatggagccagtcatgctaaatgccatatactcacactgaaacatgaggaagcaggtagatcccagaacagacaaaactttcctaaaaacatgagagtccaggctgtctgagtcagcacagtaagaaagtcctttctgctttaactcttagaaaaaagtaatatgaagtattctgaaattaaccaatcagtttatttaaatcaatttatttatattcttctgttcctggattcccattttacaaaacccactgttctactgttgtattgcccagt

SEQ ID NO: 162 - DNA, 355 bp, human
cccaggaccctgaagacgaaattgcggtgctggcctttctggtggaggctggaacccaggtgaacgagggcttccagccactggtggaggcactgtctaatatccccaaacctgagatgagcactacgatggcagagagcagcctgttggacctgctccccaaggaggagaaactgaggcactacttccgctacctgggctcactcaccacaccgacctgcgatgagaaggtcgtctggactgtgttccgggagcccattcagcttcacagagaacagatcctggcattctctcagaagctgtactacgacaaggaacagacagtgagcatgaaggacaatgtcaggcccctgca

SEQ ID NO: 163 - DNA, 283 bp, human
accacaccgacctgcgatgagaaggtcgtctggactgtgttccgggagcccattcagcttcacagagaacagatcctggcattctctcagaagctgtactacgacaaggaacagacagtgagcatgaaggacaatgtcaggcccctgcagcagctggggcagcgcacggtgataaagtccggggccccgggtcggccgctgccctgggccctgcctgccctgctgggccccatgctggcctgcctgctggccggcttcctgcgatgatggctcacttctgcac

SEQ ID NO: 164 - DNA, 337 bp, human
actgactggacgcacgtggaaaggagctatttttggaggctttaagagtaaagaatctgtcccgaaacttgtggctgactttatggctaagaagttttcactggatgcattaataacaaatattttaccttttgaaaaaataaatgaaggatttgacctgcttcgctctggaaagagtatccgtaccgtcctgacgttttgaaacaatacagatgccttcccttgtagcagttttcagcctcctctaccctacatgatctggagcaacagctaggaaatatcattaattctgctcttcagagatgttaaaaataaattacacgtgggagctttccaa

SEQ ID NO: 165 - DNA, 498 bp, human
gttggtcactgttatttgcctactgctggaagaaggacaaccgctggcaaggtagcgttccccaatctgaatacctgcaggctcccacatgagggagagggcagactcaggtgggaagatgtgccatgcgtaaggcatcaacgtgtatctgtgggatcttcgttgccttcagtaatcagggtgtgaaaaaagcagacaagttgtgtgtgtgtgtgtgtgtctaagaaaacttgtgtgcttttcaaaaaggcagtgctaagcacaagatttcaagaaagcctcttcttgttgcctagctgagtgggagagtcattttccccagacactacatttggatacaggtgccaaagaacattattaaggaattatttagaaacaatgtgtctagtttaagaaagtggttttcagtattgtgacaatacaacgtttttacaaggttgttttctaccaccatattttaaagatatttttatgaccgtgtatactcacactttgctt

SEQ ID NO: 166 - DNA, 489 bp, human
aaagcccttcatctaatatttgttgctattgccaatttttcaatgaaatgacctaaaaacaacaaaaaaaaataacctatacggtagttgctttagggggtggggggatgctatctgttagtgcttaaaagggggtaaatgcttgccgctttagaggtggatggtgctcataaaaggccccagtcgggggtatttaaaaaggactgaacagaaatccttagctagtagaatggcagcacgctgtaaaattattactgtattgtgtactggctataagatgtagacacctttcagtaagccaatcatttgtaaccattctagcagtgtcatattaggttaataaggctgctgtgttttaaagggcatttttatttgggttttggtgaaattctttaatttgttgattatattcacataaaatcagcattcattgacacatagctctaatgacatatgtatgaaaaaccatacactggatgacctagtcga

SEQ ID NO: 167 - DNA, 556 bp, human
ctgatggttctttctctgatgagatgaacaccattcttgataatcttgccgccagggactttataaactggttgattcagaccaaaatcactgacaggaaataactatatcactattcaagatcatcttcacaacatcacctgctagccacgtgggatgtttgaaatgttaagtcctgtaaatttaagaggtgtattctgaggccacattgctttgcatgccaataaataaattttcttttagtgttgtgtagccaaaaattacaaatggaataaagttttatcaaaatattgctaaaatatcagctttaaaatatgaaagtgctagattctgttattttcttcttattttggatgaagtaccccaacctgtttacatttagcgataaaattatttttctatgatataatttgtaaatgtaaattattccgatctgacatatctgcattataataataggagaatagaagaactggtagccacagtggtgaaattggaaagagaactttcttcctgaaacctttgtcttaaaaatactcagctttcaatgtatcaa

SEQ ID NO: 168 - DNA, 244 bp, human
gccggctcctgcaagtgcaaaaagtgcaaatgcacctcctgcaagaagagctgctgctcctgttgccccctgggctgtgccaagtgtgcccagggctgcatctgcaaaggggcgtcagagaagtgcagctgctgtgcctgatgtcgggacagccctgctgtcagatgaaaacagaatgacacgtaaaatccgaggttttttttttctacaactccgactcatttgctacattcctttttttctg

SEQ ID NO: 169 - DNA, 557 bp, human
ttccctgctggaggatcccaagattaaggagattgctgcaaagcacaaaaaaaccgcagcccaggttctgatccgtttccatatccagaggaatgtgattgtcatccccaagtctgtgacaccagcacgcattgttgagaacattcaggtctttgactttaaattgagtgatgaggagatggcaaccatactcagcttcaacagaaactggagggcctgtaacgtgttgcaatcctctcatttggaagactatcccttcaatgcagaatattgaggttgaatctcctggtgagattatacaggagattctctttcttcgctgaagtgtgactacctccactcatgtcccattttagccaagcttatttaagatcacagtgaacttagtcctgttatagacgagaatcgaggtgctgttttagacatttatttctgtatgttcaactaggatcagaatatcacagaaaagcatggcttgaataaggaaatgacaattttttccacttatctgatcagaacaaatgtttattaagcatcagaaactctgccaacactga

SEQ ID NO: 170 - DNA, 268 bp, human
taagtccagtagtagcaaagccccaaatcaaagccagcaagaccacagtcacaggagataaggactctgtgaacctgacctgctccacaaatgacactggaatctccatccgttggttcttcaaaaaccagagtctcccgtcctcggagaggatgaagctgtcccagggcaacaccaccctcagcataaaccctgtcaagagggaggatgctgggacgtattggtgtgaggtcttcaacccaatcagtaagaaccaaagcgaccccat

SEQ ID NO: 171 - DNA, 459 bp, human
tgagcctggggttctggtgttagaatatttttaagtaggctttactgagagaaactaaatattggcatacgttatcagcaacttcccctgttcaatagtatgggaaaaataagatgactgggaaaaagacacacccacaccgtagaacatatattaatctactggcgaatgggaaaggagaccattttcttagaaagcaaataaacttgatttttttaaatctaaaatttacattaatgagtgcaaaataacacataaaatgaaaattcacacatcacatttttctggaaaacagacggattttacttctggagacatggcatacggttactgacttatgagctaccaaaactaaattctttctctgctattaactggctagaagacattcatctatttttcaaatgttctttcaaaacatttttataagtaatgtttgtatctatttcatgctttact

SEQ ID NO: 172 - DNA, 482 bp, human
atttctttggcagttttcgtgctaatgtttttgctaaggaagataagctctgaaccattaaaggacgagtttaaaaacacaggatcaggtctcctgggcatggctaacattgacctggaaaagagcaggactggtgatgaaattattcttccgagaggcctcgagtacacggtggaagaatgcacctgtgaagactgcatcaagagcaaaccgaaggtcgactctgaccattgctttccactcccagctatggaggaaggcgcaaccattcttgtcaccacgaaaacgaatgactattgcaagagcctgccagctgctttgagtgctacggagatagagaaatcaatttctgctaggtaattaaccatttcgactcgagcagtgccactttaaaaatcttttgtcagaatagatgatgtgtcagatctctttaggatgactgtatttttcagttgccgatacagctttttgtcctctaactg

SEQ ID NO: 173 - DNA, 519 bp, human
gaagacactaccaacatgatattacgtattgatctgaccacacacaatgttactctagaagaaccaatagaaatcaactggtcatgaagatcaccatcaattttagttgtcaatgggaaaaaacaccaggatttaagtttcacagcacttacaattttccctcttcacttggttcttgtactctacaaaatatagctttcataacatcgaaaagttattttgtagcgtacatcaatgataatgctaattttattatagtaatgtgacttggattcaattttaaggcatatttaacaaaatttgaatagccctatttatccttgttaagtatcagctacaattgtaaactagttactaaacatgtatgtaaatagctaagatataatttaaacgtgatttttaaattaaataaaatttttatgtaattatatatactatatttttctcaatgtttagcagatttaagatatgtaacaacaattatttgaagatttaattacttcttagtatgtgcattta

SEQ ID NO: 174 - DNA, 227 bp, human
attgcaatttcttaggtaaccttatatttacaataaatgaagattaccctcaaatgctagaagctgtctaggtccgtccggtgtgtcagatttcctcagattagatgtgccaataaccaagtttattcagtaaacaacttgtacttgtttcatctggtttattactctcacccataaacagtaatgactctctgaccctctggaaatatgtaatgcttccaatcttg

SEQ ID NO: 175 - DNA, 478 bp, human
ttttggacctgcggtggtggccaaccactggaacttccactggatctactggctgggcccactcctggctggcctgcttgttggactgctcattaggtgcttcattggagatgggaagacccgcctcatcctgaaggctcggtgagcagagctcgtgggattcctgctgctccaggtgtcctcagctcacctgtcccagactgaggacaggggagttcctgcatttcctgccagggcagaggcccagaggagcgaccccctgcttccactgcttgggcctgctttctcagatagactgactgctgaggaggctctaggttcttggaattcctttgtgctcatcagagaccccagcctggggaacacgctgcccgcactgcccagagagcagtgcaaacaccacaacacgagcgtgtttcttgagaggaatgtccccgagttggacaaggaggctgtttctgcacatcagctcatttcc

SEQ ID NO: 176 - DNA, 436 bp, human
gaggggtcaccgtgcaggatggaaatttctccttttctctggagtcagtgaagaagctcaaagacctccaggagccccaggagcccagggttgggaaactcaggaactttgcacccatccctggtgaacctgtggttcccatcctctgtagcaacccgaactttccagaagaactcaagcctctctgcaaggagcccaatgcccaggagatacttcagaggctggaggaaatcgctgaggacccgggcacatgtgaaatctgtgcctacgctgcctgtaccggatgctaggggggcttgcccactgcctgcctcccctccgcagcagggaagctcttttctcctgcagaaagggccacccatgatactccactcccagcagctcaacctaccctggtccagtcgggaggagcagcccggggaggaactgggtgact

SEQ ID NO: 177 - DNA, 234 bp, human
tcgtctgcttcacaagctatcgctatggtgttcgtgcgcaggccgtggcccgccttgaccacagtgcttctggccctgctcgtctgcctaggggcgctggtcgacgcctaccccatcaaacccgaggctcccggcgaagacgcctcgccggaggagctgaaccgctactacgcctccctgcgccactacctcaacctggtcacccggcagcggtatgggaaaagagacggcccg

SEQ ID NO: 178 - DNA, 287 bp, human
tatttgaatatgtatcgtgccccctccggtgtctttgatcaggatgacatgtgccatttttcagaggacgtgcagacaggctggcattctagattacttttcttactctgaaacatggcctgtttgggagtgcgggattcaaaggtggtcccaccgctgcccctactgcaaatggcagttttaatcttatcttttggcttctgcagatggttgcaattgatccttaaccaataatggtcagtcctcatctctgtcctgcttcataggtgccaccttgtgtgtttaaa

SEQ ID NO: 179 - DNA, 536 bp, human
tgacgcaaaataccaccttggcgcctacacgggagacgacgtccgcatcgtccgtgacgacatgctgtgtgccgggaacacccggagggactcatgccagggcgactccggagggcccctggtgtgcaaggtgaatggcacctggctgcaggcgggcgtggtcagctggggcgagggctgtgcccagcccaaccggcctggcatctacacccgtgtcacctactacttggactggatccaccactatgtccccaaaaagccgtgagtcaggcctgggttggccacctgggtcactggaggaccaacccctgctgtccaaaacaccactgcttcctacccaggtggcgactgccccccacaccttccctgccccgtcctgagtgccccttcctgtcctaagccccctgctctcttctgagccccttcccctgtcctgaggacccttccccatcctgagcccccttccctgtcctaagcctgacgcctgcaccgggccctccggccctcccctgcccaggcagctggtggtgggcgct

SEQ ID NO: 180 - DNA, 472 bp, human
atgatcacaaaatgttgcctgttttgtttccgaaagcttgccaaaacaggaaagaagaagaaaagggattagttatatcaaaagcctgaagtggaatgaccaaaagatgggactcctcctttattccagcatggagggttttaaatggaggatttcctttttcctgcgacaaaacgtcttttcacaacttaccctgttaagtcaaaatttattttccaggaatttaatatgtactttagttggaattattctatgtcaatgatttttaagctatgaaaaataataatataaaaccttatgggcttatattgaaatttattattctaatccaaaagttaccccacacaaaagttactgagcttccttatgtttcacacattgtatttgaacacaaaacattaacaactccactcatagtatcaacattgttttgcaaatactcagaatattttggcttcattttgagcagaat

SEQ ID NO: 181 - DNA, 334 bp, human
caagtatcggggctctgctatcaaggtgcaaagtccttcgtggatgcaacctcaaccatatattctacagcaccctggtgccgtgttaactccctcaatggagcacaccatgtcactacagcccgcatcaatgatcagccctctggcccagcagatgagtcatctgtcactaggcagcaccggaacatacatgcctgcaacgtcagctatgcaaggagcctacttgccacagtatgcacatatgcagacgacagcggttcctgttgaggaggcaagtggtcaacagcaggtggctgtcgagacgtctaatgaccattctccatatacctttcaa

SEQ ID NO: 182 - DNA, 475 bp, human
tggactggtgtcgagccaagactcggggcctacgagcacgtcgacatccagaacttctcctccagctggagtgatgggatggccttctgtgccctggtgcacaacttcttccctgaggccttcgactatgggcagcttagccctcagaaccgacgccagaacttcgaggtggccttctcatctgcggagatgctggtggactatgtgcccctggtggaggtggacgacatgatgatcatgggcaagaagcctgaccccaagtgtgtcttcacctatgtgcagtcgctctacaaccacctgcgacgccacgaactggcctcgcgcggcaagaatgtctagcctgcccgcccgcatggccagccagtggcaactgccgcccccactctccgggcaccgtctcctgcctgtgcgtccgcccaccgctgccctgtctgttgcgacaccctcccccccacatacacacgcagcgttttga

SEQ ID NO: 183 - DNA, 561 bp, human
ggatgtgatagcattcctgctggcctgcgtggcaactgtgatatttatcatcacaaaattttgcctgttttgtttccgaaagcttgccaaaacaggaaagaagaagaaaagagattagttatatcaaaagcctgaagtggaatgactgaaagatgggactcctcctttatttcagcatggagggttttaaatggaggatttcctttttcctgtgacaaaacatcttttcacaacttaccttgttaagacaaaatttattttccagggatttaatacgtactttagttggaattattctatgtcaatgatttttaagctatgaaaaatacaatggggggaaggatagcatttggagatatacctaatgttaaatgacgagttactggatgcagcacgcaacatggcacatgtgtatacatatgtagctaacccttcgttgtgcacatgtaccctaaaacttaaagtataatttaaaaaaagcaaaaaaaaaaaataccaactcttttttttaaaccaggaaggaaaatgtgaacatggaaacaacttctagtattggatctg

SEQ ID NO: 184 - DNA, 512 bp, human
gccccggagtgcagctgcggggacccgctgctcgaccccggcctgccggagcccgaggccccgccccctgcgggtcccgaaccgcttaccctcatccctgggcctgtcgagcccttcagcatcgtgaccatgcccgggccccggggtccggcgccaccctggctgcccagccctattggcgaggaggaggagaatctggcctgagatcttagagcccagccccctaaggacagggaaccaggtccctgcacggcacccacgcaggtgtcccggtctgcataagcctcgtatgcctttgtaaagtccacctacacttttgaccagctctcgctgcccgcatgtgtttggcgctgtgctaggggcgggagttcttccagactcttggaccagcccgccctgaccaccagctctacttcccaacccccactgcctgagaggtctctatcagtgtcctgcctgaattctttccttcaagtgaagatgtgactgactacctcctcgagttgtcatga

SEQ ID NO: 185 - DNA, 450 bp, human
gaacccagggagcgcgatgggctgcagggctgcgtcagggctcctgccaggagtggccgtggtcctcctgctgctgctgcagagcacacagtcagtctacatccagtaccaaggcttccgggtccagctggaatccatgaagaagctgagtgacctggaggcacagtgggcacccagcccccgcctgcaggcccagagcctcctgcccgccgtgtgccaccaccctgctctgcctcaggaccttcagcctgtctgcgcctcgcaggaggcttccagcatcttcaagaccctgaggaccatcgctaacgacgactgtgagctgtgtgtgaacgttgcgtgtaccggctgcctctgagatagccctgggtaccctgagcccaccagggacacctcgcccttcagcccaccaccctggcaggcttccatccccgtccatgctcaagatgggtc

SEQ ID NO: 186 - DNA, 474 bp, human
ttattcttagcgtcactggtctggctttcagaattaacatacaaggttgccacacctagttctgcccagctttatgtcttttattccagtattccaccaaagtttgttttcctgcattccagttctcaagtcttaagataaagattgtacttgacagtttagtatatccataaaactatttgaggtggttaaggttcttgggttcattttccttaatactttgctgaatattgtagattgtaggcaatgaaaaagtctactaaattaggaaaaccttgaataattaggtatcctaggtaagagcccctaaacatcaagcaatctgtgagtctgtaaagaaataaatattttttggattattcttatctaattccacccctgttggaagatgatttctttgttctttgcaactatggaagctgtgaaaatcatcacaagtgcctctgaaagcgagtgttaggttggttagagggt

SEQ ID NO: 187 - DNA, 569 bp, human
gtgacggtgctgaagaaggccctggatgaagagacgcggtcccatgaggctcaggtccaggagatgaggcagaaacacgcacaggcggtggaggagctcacagagcagcttgagcagttcaagagggccaaggcgaacctagacaagaataagcagacgctggagaaagagaacgcagacctggccggggagctgcgggtcctgggccaggccaagcaggaggtggaacataagaagaagaagctggaggcgcaggtgcagacactgagtttttagaaaaacatatccacggtaaccggtccctggcaattctgtttacatgaaatggggagaaagtcaccgaaatgggtgccgccggcccccactcccaattcattccctaacctgcaaacctttccaacttctcacgtcaggcctttgagaattctttccccctctcctggtttccacacctcagacacgcacagttcaccaagtgccttctgtagtcacatgaattgaaaaggagacgctgctcccacggaggggagcaggaatgctgcactgtttacaccctgactgtgcttaaa

SEQ ID NO: 188 - DNA, 422 bp, human
tagtggccgtgaggagcatcttcagtaagaaggaaggttctgacagacaatggaactacgcctgcatgcccacgccacagagcctcggggaacccacggagtgctggtgggaggagatcaacagggctggcatggaatggtaccagacgtgctccaacaatgggctggtggcaggattccagagccgctacttcgagtcagtgctggatcgggagtggcagttttactgttgtcgctacagcaagaggtgcccatattcctgctggctaacaacagaatatccaggtcactatggtgaggaaatggacatgatttcctacaattatgattactatatccgaggagcaacaaccactttctctgcagtggaaagggatcgccagtggaagttcataatgtgccggatgactgaatacgactgt

SEQ ID NO: 189 - DNA, 183 bp, human
aatgtcatagacactgatttcatcgacgaggaagttcttatgtccttggtgatagaaatgggtttggaccgcatcaaggagctgcccgaactctggctggggcaaaacgagtttgattttatgacggacttcgtgtgcaaacagcagcccagcagagtgagctgttgactcgatcgaaacccc

SEQ ID NO: 190 - DNA, 434 bp, human
  misc_feature (158)..(158): n is a, c, g, or t
ccccccaattgctgccaatagtggataacatgtatcactcactgccaaaaatagaaagtgaccatgaaaaataaattcgctggggaagggggctccatgctggtgtggccaaggctgagagctctctcttctctgttacaaaacgagataagcaagtnttagaattgccttaaggccacactggcatctccctgaccttctccagggacagaagcaggagtaagtttctcatcccatgggcgaccagggccatctcctcccaccagtggcccccactcacagggagctggcaatgccctacctgcctgttctccagatggagaaacaggctctgagatttcacaggtcttgcccaaagtcattgattttaatgattaaaaagaataaacacagtgtttcctgagtagcagtgattgttatgccttgctatttta

SEQ ID NO: 191 - DNA, 463 bp, human
gatgtattaacctacctgtgaatcatatgttgtaggaaaagctgttcccatgtctaacaggacttgaattcaaagcatgtcaagtggatagtagatctgtggcgatatgagagggatgcagtgcctttccccattcattcctgatggaattgttatactaggttaacatttgtaatttttttctagttgtaatgtgtatgtctggtaaataggtattatattttggccttacaataccgtaacaatgtttgtcattttgaaatacttaatgccaagtaacaatgcatgctttggaaatttggaagatggttttattctttgagaagcaaatatgtttgcattaaatgctttgattgttcatatcaagaaattgattgaacgttctcaaaccctgtttacggtacttggtaagagggagccggtttgggagagaccattgcatcgctgtccaagtgtttcttgt

SEQ ID NO: 192 - DNA, 468 bp, human
agggcaagtgttcccaaattgacgccacctaataatcatcaccacaccgggagcagatctgaaggcacactttgatttttttaaggataagaaccacagaacactgggtagtagctaatgaaattgagaagggaaatcttagcatgcctccaaaaattcacatccaatgcatactttgttcaaatttaaggttactcaggcattgatcttttcagtgttttttcacttagctatgtggattagctagaatgcacaccaaaaagatacttgagctgtatatatatatgtgtgtgtgtgtgtgtgtgtgtgtgtgtgtgcatgtatgtgcacatgtgtctgtgtgatatttggtatgtgtatttgtatgtactgttattcaaaatatatttaatacctttggaaaatcttgggcaagatgacctactagttttccttgaaaaaaagttgctttgttattaatattgtgct

SEQ ID NO: 193 - DNA, 531 bp, human
ccgagccgagcttactgtgagtgtggagatgttatcccaccatgtaaagtcgcctgcgcaggggagggctgcccatctccccaacccagtcacagagagataggaaacggcatttgagtgggtgtccagggccccgtagagagacatttaagatggtgtatgacagagcattggccttgaccaaatgttaaatcctctgtgtgtatttcataagttattacaggtataaaagtgatgacctatcatgaggaaatgaaagtggctgatttgctggtaggattttgtacagtttagagaagcgattatttattgtgaaactgttctccactccaactcctttatgtggatctgttcaaagtagtcactgtatatacgtatagagaggtagataggtaggtagattttaaattgcattctgaatacaaactcatactccttagagcttgaattacatttttaaaatgcatatgtgctgtttggcaccgtggcaagatggtatcagagagaaacccatcaattgctcaaatactc

SEQ ID NO: 194 - DNA, 392 bp, human
ggagctgtcaccatgacgggggaacttgaggttaagaacatggacatgaagccggggtcaaccctgaagatcacaggcagcatcgccgatggcactgatggctttgtaattaatctgggccaggggacagacaagctgaacctgcatttcaaccctcgcttcagcgaatccaccattgtctgcaactcattggacggcagcaactgggggcaagaacaacgggaagatcacctgtgcttcagcccagggtcagaggtcaagttcacagtgacctttgagagtgacaaattcaaggtgaagctgccagatgggcacgagctgacttttcccaacaggctgggtcacagccacctgagctacctgagcgtaaggggcgggttcaacatgtcctc

SEQ ID NO: 195 - DNA, 233 bp, human
gcgtgttttcctcttgatcgggaactcctgcttctccttgcctcgaaatggaccccaactgctcctgctcgcctgttggctcctgtgcctgtgccggctcctgcaaatgcaaagagtgcaaatgcacctcctgcaagaagagctgctgctcctgctgccctgtcggctgtgccaagtgtgcccagggctgcatctgcaaagggacgtcagacaagtgcagctgctgtgcctga

SEQ ID NO: 196 - DNA, 578 bp, human
cccgaccatcgaatcttgcgaacaacacgatacttgttaagtggctaccccaaaacgatctgcttggtcacccgatgacccgtgcctttatcacccatgctggttcccatggtgtttatgaaagcatatgcaatggcgttcccatggtgatgatgcccttgtttggtgatcagatggacaatgcaaagcgcatggagactaagggagctggagtgaccctgaatgttctggaaatgacttctgaagatttagaaaatgctctaaaagcagtcatcaatgacaaaagttacaaggagaacatcatgcgcctctccagccttcacaaggaccgcccggtggagccgctggacctggccgtgttctgggtggagtttgtgatgaggcacaagggcgcgccacacctgcgccccgcagcccacgacctcacctggtaccagtaccattccttggacgtgattggtttcctcttggccgtcgtgctgacagtggccttcatcacctttaaatgttgtgcttatggctaccggaaatgcttggggaaaaaagggcgagttaagaaagcccacaaatccaagacc

SEQ ID NO: 197 - DNA, 509 bp, human
tgcagaggcctatgttttcactcctaacatgatctgtgctggaggagagaagggcatggatagctgtaaaggggacagtggtggggcctttgctgtacaggatcccaatgacaagaccaaattctacgcagctggcctggtgtcctgggggccccagtgtgggacctatgggctctacacacgggtaaagaactatgttgactggataatgaagactatgcaggaaaatagcaccccccgtgaggactaatccagatacatcccaccagcctctccaagggtggtgaccaatgcattaccttctgttccttatgatattctcattatttcatcatgactgaaagaagacacgagcgaatgatttaaatagaacttgattgttgagacgccttgctagaggtagagtttgatcatagaattgtgctggtcatacatttgtggtctgactccttggggtcctttccccggagtacctattgtagataacactatgggtggggcactccttt

SEQ ID NO: 198 - DNA, 507 bp, human
gggcctaaccagccttgggagtattgactggtcccttacctcttatggctaagtctttgactgtgttcatttaccaagttgacccagtttgtcttttaggttaagtaagactcgagagtaaaggcaaggaggggggccagcctctgaatgcggccacggatgccttgctgctgcaaccctttccccagctgtccactgaaacgtgaagtcctgttttgaatgccaaacccaccattcactggtgctgactacatagaatggggttgagagaagatcagtttgggcttcacagtgtcatttgaaaacgttttttgttttgttttgtaattattgtggaaaactttcaagtgaacagaaggatggtgtcctactgtggatgagggatgaacaaggggatggctttgatccaatggagcctgggaggtgtgcccagaaagcttgtctgtagcgggttttgtgagagtgaacactttccactttttgacaccttatcctgatgtatggttc

SEQ ID NO: 199 - DNA, 510 bp, human
agatgtgtttagaacctcttgtttaaaaataatagactgcttatcataaaatcacatctcacacatttgaggcagtggtcaaacaggtaaagcctatgatgtgtgtcattttaaagtgtcggaatttagcctctgaataccttctccattgggggaaagatattcttggaaccactcatgacatatcttagaaggtcattgacaatgtataaactaattgttggtttgatatttatgtaaatatcagtttaccatgctttaattttgcacattcgtactatagggagcctattggttctctattagtcttgtgggttttctgtttgaaaaggagtcatggcatctgtttacatttaccttatcaaacctagaatgtgtatatttataaatgtatgtcttcattgctaggtactaatttgcagatgtctttacatatttcaatacagaaactataacattcaatagtgtgctgtcaaagtgtgcttagctcacctggatatacctacattg

SEQ ID NO: 200 - DNA, 524 bp, human
  misc_feature (314)..(320): n is a, c, g, or t
gaagggagtgttgctcccagtccagaggcctgattctgttcggactgggttctcaagacacgaccaggttctcaagacacgagtccccttgttcctccccattaaagggggtttgtcagaagcaagaacagcccctctccccagtcacagcctgaagggaggccccgagagcttcctccttccccccacctgctccttaccttctctgccctgctttttagaactgcagttcattgttttaagggattgggggagggagcctggggacacaaaccttttatacaatacaaagctttgctttttttttttttttnnnnnnnccttttccctttctcggttctcttctctcctctgaatggctgaagacccctctgccgagggaggttggggattgtgggacaaggtcccttggtgctgatggcctgaaggggcctgagctgtgggcagatgcagttttctgtgggcttggggaacctctcacgttgctgtgtcctggtgagcagcccgaccaataaacctgcttt

SEQ ID NO: 201 - DNA, 406 bp, human
ggctgcctgcggatgaaggaccagtgtgacaagtgccgggagatcttgtctgtggactgttccaccaacaacccctcccaggctaagctgcggcgggagctcgacgaatccctccaggtcgctgagaggttgaccaggaaatataacgagctgctaaagtcctaccagtggaagatgctcaacacctcctccttgctggagcagctgaacgagcagtttaactgggtgtcccggctggcaaacctcacgcaaggcgaagaccagtactatctgcgggtcaccacggtggcttcccacacttctgactcggacgttccttccggtgtcactgaggtggtcgtgaagctctttgactctgatcccatcactgtgacggtccctgtagaagtctccaggaagaacccta

SEQ ID NO: 202 - DNA, 286 bp, human
agcagctgaacgagcagtttaactgggtgtcccggctggcaaacctcacgcaaggcgaagaccagtactatctgcgggtcaccacggtggcttcccacacttctgactcggacgttccttccggtgtcactgaggtggtcgtgaagctctttgactctgatcccatcactgtgacggtccctgtagaagtctccaggaagaaccctaaatttatggagaccgtggcggagaaagcgctgcaggaataccgcaaaaagcaccgggaggagtgagatgtggatgttgc

SEQ ID NO: 203 - DNA, 101 bp, human
cgatcaccaatgtacctccagaggtaactgtgctcacgaacagccctgtggaactgagagagcccaacgtcctcatctgtttcatagacaagttcacccca

SEQ ID NO: 204 - DNA, 530 bp, human
  misc_feature (115)..(115): n is a, c, g, or t
  misc_feature (117)..(117): n is a, c, g, or t
  misc_feature (121)..(123): n is a, c, g, or t
gatataccatgtattcacactttaaaaaatacacatagcagagtatcggaaagtatgtaccaaaacattgatctggctaccactgggagtcaggatcatgagttcatcttcttcntncttnnnactgcttttccaaacattctccagtaagcaggtactacatttataatggaaggaattttttaaaaaattttaagctgtacactttcaagtaagatctgaattctaatgctggcttgtgcctcttactatgtggtttggtcattatagataatgcccagtttcagtttccccatctttgaaaaggagataatatgttatctcttgggagtagttctgaatatgaagtgtgttgatacaagaagcactgacaatgtttctgtttcttaaatttaaaactggcctggtttgccttttttatcaagagagcttaacagataaaaaatgaaattagtctattttctacttgccagcagagtatctgtcttattttaggatgcagtgtgaaacttaccattcaactgaacaac

SEQ ID NO: 205 - DNA, 480 bp, human
  misc_feature (431)..(431): n is a, c, g, or t
gaaggctggattctatctacataagtcctttcaattccaccagggccagagcagctccaccactgtgcacttagccatgatggcaacagaaaccaagagacacaattacgcaggtatttagaagcagagggacaaccagaaggcccttaactatcaccagtgcatcacatctgcacactctcttctccattccctagcaggaacttctagctcatttaacagataaagaaactgaggcccacggtttcagctagacaatgatttggccaggcctagtaaccaaggccctgtctctggctactccctggaccacgaggctgattcctctcatttccagcttctcagtttctgcctgggcaatggccaggggccaggagtggggagagttgtgatggaggggagaggggtcacacccaccccctgcctggttntaggctgctgcacaccaaggccctgcatctgtctgctctgcatatatgt

SEQ ID NO: 206 - DNA, 546 bp, human
agacagctgtttagggtcttctcccctcacccatgctttcatcatcccctccgcacagcctccccgtccaggccttctaaccacacctacccagggctgccgcattcctgcactcagaagtctgcagcggtgcctcacaaacttgattgtgcataaaaatcactggggatcttgttaatacagattctaactcaatagatctgggagatcctgcatttctaacaagctcccaggtaaggcggaggctgctggtgtgaggaccatgctgtgagcagcagggcgagagtgcccagggctgatatatattggaaatatcacccctgaagccatcgctggcccccacctcctgtggactgatgccccagggattcccaccccacttctgcaaccccaggtatccttcattatccaccccatcccagactcccaccccagggattgcccgtgaagactttggcctagcaaattgtgttggttatgtgagtgttgttttaatcagagatgtacatgattgccaatctgcatttcttaccagtgtgaccacac

SEQ ID NO: 207 - DNA, 562 bp, human
atcatcagatcgactcttatacgagtaatatgcctgctatttgaagtgtaattgagaaggaaaattttagcgtgctcactgacctgcctgtagccccagtgacagctaggatgtgcattctccagccatcaagagactgagtcaagttgttccttaagtcagaacagcagactcagctctgacattctgattcgaatgacactgttcaggaatcggaatcctgtcgattagactggacagcttgtggcaagtgaatttgcctgtaacaagccagattttttaaaatttatattgtaaatattgtgtgtgtgtgtgtgtgtgtatatatatatatatatgtacagttatctaagttaatttaaagttgtttgtgcctttttatttttgtttttaatgctttgatatttcaatgttagcctcaatttctgaacaccataggtagaatgtaaagcttgtctgatcgttcaaagcatgaaatggatacttatatggaaattctgctcagatagaatgacagtccgtcaaaacagattgtttgcaaaggggaggcatcagtgtcttg

SEQ ID NO: 208 - DNA, 553 bp, human
tacaccacaatggctgagcacttcctgacgttgctggtagtgcctgccatcaagaaagattatggttcccaggaagacttcactcaagtgtggaacaccaccatgaaagggctcaagtgctgtggcttcaccaactatacggattttgaggactcaccctacttcaaagagaacagtgcctttcccccattctgttgcaatgacaacgtcaccaacacagccaatgaaacctgcaccaagcaaaaggctcacgaccaaaaagtagagggttgcttcaatcagcttttgtatgacatccgaactaatgcagtcaccgtgggtggtgtggcagctggaattgggggcctcgagctggctgccatgattgtttccatgtatctgtactgcaatctacaataagtccacttctgcctctgccactactgctgccacatgggaaactgtgaagaggcaccctgggcaagcagcagtgattgggggaggggacaggatctaacaatgtcacttgggccagaatggacctggcctttctgctcccagacttgggggctag

SEQ ID NO: 209 - DNA, 220 bp, human
  misc_feature (132)..(132): n is a, c, g, or t
  misc_feature (135)..(135): n is a, c, g, or t
  misc_feature (159)..(159): n is a, c, g, or t
  misc_feature (162)..(162): n is a, c, g, or t
  misc_feature (172)..(172): n is a, c, g, or t
  misc_feature (174)..(174): n is a, c, g, or t
  misc_feature (180)..(180): n is a, c, g, or t
gcagttatgggcaaccctaaggtgaaggctcatggcaagaaagtgctcggtgcctttagtgatggcctggctcacctggacaacctcaagggcacctttgccacactgagtgagctgcactgtgacaagctncangtggatcctgagaacttcaggctnctnggcaacgtgntngtctgngtgctggcccatcactttggcaaagaattcaccccaccag

SEQ ID NO: 210 - DNA, 417 bp, human
tctctgggctccaggctgaggacgaggctgattattactgctgctcatatgcaggtagttacactgtggttttcggcggagggaccaaactgaccgtcctaggtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgtt

SEQ ID NO: 211 - DNA, 532 bp, human
tttggtgcagctgctagtcagtccctgactgacattgccaagtattcaataggcagactgcggcctcacttcttggatgtttgtgatccagattggtcaaaaatcaactgcagcgatggttacattgaatactacatatgtcgagggaatgcagaaagagttaaggaaggcaggttgtccttctattcaggccactcttcgttttccatgtactgcatgctgtttgtggcactttatcttcaagccaggatgaagggagactgggcaagactcttacgccccacactgcaatttggtcttgttgccgtatccatttatgtgggcctttctcgagtttctgattataaacaccactggagcgatgtgttgactggactcattcagggagctctggttgcaatattagttgctgtatatgtatcggatttcttcaaagaaagaacttcttttaaagaaagaaaagaggaggactctcatacaactctgcatgaaacaccaacaactgggaatcactatccgagcaatcaccagc

SEQ ID NO: 212 - DNA, 361 bp, human
gcacgccatcaatgccatcgtgcgcagcccgcgtggcggggcccggaggcacgcagagctgtccttcgtgttcctcacggacggcgtcacgggcaacgacagtctgcacgagtcggcgcactccatgcgcaacgagaacgtggtacccaccgtcctggccttgggcagcgacgtggacatggacgtgctcaccacgctcagcctgggtgaccgcgccgccgtgttccacgagaaggactatgacagcctggcgcaacccggcttcttcgaccgcttcatccgctggatctgctagcgccgccgcccgggccccgcagtcgagggtcgtgagcccaccccgtccatggtgctaagcgggccc

SEQ ID NO: 213 - DNA, 498 bp, human
tgtttcctcatagctagttctcaagctgcatgtaagattttaacgggaagagaaaataggcctggacctgaaggtctcaaatatgttgagaagaaagtatgaactataaggaacttgagatgtagatttattttgcaggaaatacgaggaaaataggaaagaagtgtttgccgcatcaagtgtaaagaatgatactgaggatttttacattttatgaaatgaaataatggcatttacaaaatgaaaaatgtagtttcacaactaagttttgttaacagagactgcatgctttgcttatagttcttaattttggttttgacattcatttaattttttccatgttaaatatgtagtttaattatttactcaaaataaacattgttcatgcttttaggcctttgggggaattgatttttatccacaggtagaaaatggtctttgcacacactacacttatttcaaatatacaatgtgctcccgaactttcgcattagtctt

SEQ ID NO: 214 - DNA, 491 bp, human
gaaatatcactgacactccagactaaagcagagtctaggtttctgcaattttgttacagtaatttgtaaatagctttagtaaactcaccttgcatggtagattaataagatgacttactgtacatgaattacacaataatgagatctggtggctatttccacattttgaaaaggattcagttatttactgacagtggtgagcatcctttttaaaataatgttctgatacttaaacattagagagcagtatctttaaatgaattattaacactttggaatacttacattttctgttatttttgattgcctgataaccagtttcaatgatgaaaatgaaaacaagtgctgaagatgaaatggaagagaaccgttttaatctggattttgttttgtcacacctggaaaatactttgcaaatatgttctaaattgaaaacaatttttttatgatcacatggttcactaccaaatgaccctcaaataagccagatg

SEQ ID NO: 215 - DNA, 389 bp, human
gatagtcggaatagagccgccccaactcagatcctacaacacgcaagttccttcttgaaccctggtgcctcctaccctatggccctgaatggtgcactggtttaattgtgttggtgtcggcccctcacaaatgcagccaagtcatgtaattagtcatctggaacaaagactaaaaacagcagagaattgcgggttctacccagtcagaagatcacaccatggagactttctactagaggacttgaaagagaactgaggggccacaaaataaacttcaccttccattaagtgttcaagcatgtctgcaaattaggagggagttagaaacagtctttttcatcctttgtgatgaagcctgaaattgtgccgtgttgccttatatgaatatg

SEQ ID NO: 216 - DNA, 515 bp, human
tgtctccccgctatctaaaaaagtataagaacaagcagataacagcgagaatcttggaggcccatcagaatgtagctcagatgagtctaattgaagccaagatgagatttattcaagcttggcagtcactacctgaatttggcatcactcacttcattgcaaggttccaagggggcaaaaaagaagaacttattggaattgcatacaacagactgattcggatggatgccagcactggagatgcaattaaaacatggcgtttcagcaacatgaaacagtggaatgtcaactgggaaatcaaaatggtcaccgtagagtttgcagatgaagtacgattgtccttcatttgtactgaagtagattgcaaagtggttcatgaattcattggtggctacatatttctctcaacacgtgcaaaagaccaaaacgagagtttagatgaagagatgttctacaaacttaccagtggttgggtgtgaatagaaatactgtttaatgaaactccacggccataa

SEQ ID NO: 217 - DNA, 536 bp, human
  misc_feature (26)..(26): n is a, c, g, or t
  misc_feature (64)..(64): n is a, c, g, or t
  misc_feature (124)..(124): n is a, c, g, or t
aaaatgctattagtccgtcgtgcttnatttgtttttgtccttgaataagcatgttatgtatatngtctcgtgtttttatttttacaccatattgtattacacttttagtattcaccagcataancactgtctgcctaaaatatgcaactctttgcattacaatatgaagtaaagttctatgaagtatgcattttgtgtaactaatgtaaaaacacaaattttataaaattgtacagttttttaaaaactactcacaactagcagatggcttaaatgtagcaatctctgcgttaattaaatgcctttaagagatataattaacgtgcagttttaatatctactaaattaagaatgacttcattatgatcatgatttgccacaatgtccttaactctaatgcctggactggccatgttctagtctgttgcgctgttacaatctgtattggtgctagtcagaaaattcctagctcacatagcccaaaagggtgcgagggagaggtggattaccagtattgttcaataatccatggttca

SEQ ID NO: 218 - DNA, 514 bp, human
gaccagttcttcggagagcacctgttggagtctgatcttttcccgacgtctacttccctgagtcccttctaccttcggccaccctccttcctgcgggcacccagctggtttgacactggactctcagagatgcgcctggagaaggacaggttctctgtcaacctggatgtgaagcacttctccccagaggaactcaaagttaaggtgttgggagatgtgattgaggtgcatggaaaacatgaagagcgccaggatgaacatggtttcatctccagggagttccacaggaaataccggatcccagctgatgtagaccctctcaccattacttcatccctgtcatctgatggggtcctcactgtgaatggaccaaggaaacaggtctctggccctgagcgcaccattcccatcacccgtgaaaagaagcctgctgtcaccgcagcccccaagaaaaagatgccctttcttgaattgcattttttaaaacaagaaagtttccccaccagtgaatgaa

SEQ ID NO: 219 - DNA, 293 bp, human
tgaatcttcgggtgtttccctttagctaagcacagatctaccttggtgatttggaccctggttgctttgtgtctagttttctagacccttcatctcttacttgatagacttactaataaaatgtgaagactagaccaattgtcatgcttgacacaactgctgtggctggttggtgctttgtttatggtagtagtttttctgtaacacagaatataggataagaaataagaataaagtaccttgactttgttcacagcatgtagggtgatgagcactcacaattgttgactaaa

SEQ ID NO: 220 - DNA, 398 bp, human
ctgtgtatccttcaaagacccagcccctgcagcaccataacctcctggtctgttctgtgagtggtttctatccaggcagcattgaagtcaggtggttccggaatggccaggaagagaagactggggtggtgtccacaggcctgatccacaatggagactggaccttccagaccctggtgatgctggaaacagttcctcggagtggagaggtttacacctgccaagtggagcacccaagcgtgacaagccctctcacagtggaatggagagcacggtctgaatctgcacagagcaagatgctgagtggagtcgggggctttgtgctgggcctgctcttccttggggccgggctgttcatctacttcaggaatcagaaaggacactctggacttcagcca

SEQ ID NO: 221 - DNA, 396 bp, human
  misc_feature (148)..(149): n is a, c, g, or t
  misc_feature (151)..(154): n is a, c, g, or t
  misc_feature (156)..(156): n is a, c, g, or t
agtcaaatgccaaacactagctctgtattaatccccatcattactggtaaagcctcatttgaatgtgtgaattcaatacaggctatgtaaaatttttactaatgtcattattttgaaaaaataaatttaaaaatacattcaaaattannannnnanacaagcttaattgttaatattccctaaacacaattttatgaagggagaagacattggtttgttgacaataacagtacatcttttcaagttctcagctatttcttctacctctccctatcttacatttgagtatggtaacttatgtcatctatgttgaatgtaagcttataaagcacaaagcatacatttcctgactggtctagagaactgatgtttcaatttacccctctgctaaataaa

SEQ ID NO: 222 - DNA, 396 bp, human
ggcaaactgcttaatcttgtggattttgtagatggtttcaaatgactgaactgcattcagatttacgagtgaaaggaaaaattgcattagttggttgcatgaactttgaagggcagatattactgcacaaactgccatctcgcttcatttttttaactatgcatttgagtacagactaatttttaaaatatgctaaactggaagattaaacagatgtggcccaaactgttctggatcaggaaagtcatactgttcactttcaagttggctgtcccccccgccgcccccccccacccccatatgtacagatgataatagggtgtggaatgtcgtcagtggcaaacatttcacagattattttgtttctgtcttcaacatttttgacactgtgctaat

SEQ ID NO: 223 - DNA, 479 bp, human
ctcctccatgagtctgacatctcggaaactgagcagctgccggacgcctgggtcaggaatccaagaccccacctcttaaggactggttcctcagaaagcaccctcagggaaaaaggtgaaaacattacatccgtggattctcctgccacaaccgcattggaagaaaaggctgccgcaacatctcagcgaggagtgaaggacccatgtcccaggaaccgcgctgcgccacctgcactcacccccctcacattctcttaagcacccggtggccctccgaggcctggcggaatggtggtgcccacggggttgggcaagggctcaccaggacctcaacgggcaaagttgtgcacactaaaatatcaaatcaaggtgcttggttttaaagtaaatgtttttctaaagaaagctgtgttcttctgttgacccagacgaatagggcacagccctgtaactgcacgtgccttctgtcattgggaatg

SEQ ID NO: 224 - DNA, 259 bp, human
caccatcacgtgcctggtgacgggcttctctcccgcggacgtcttcgtgcagtggatgcagagggggcagcccttgtccccggagaagtatgtgaccagcgccccaatgcctgagccccaggccccaggccggtacttcgcccacagcatcctgaccgtgtccgaagaggaatggaacacgggggagacctacacctgcgtggtggcccatgaggccctgcccaacagggtcaccgagaggaccgtggacaagtccacc

SEQ ID NO: 225 - DNA, 340 bp, human
  misc_feature (208)..(208): n is a, c, g, or t
  misc_feature (262)..(262): n is a, c, g, or t
ttcttttttatattgtcctccacctccatcattttcaataaaagatagggcttttgctcccttgttcttggagggaccattattacatctctgaactacctttgtatccaacatgttttaaatccttaaatgaattgctttctcccaaaaaaagcacaatataaagaaacacaagatttaattatttttctacttggggggaaaaaangtcctcatgtagaagcacccacttttgcaatgttgttctaagctatctatctanctctcagcccatgataaagttccttaagctggtgattcctaatcaaggacaagccaccctagtgtctcatgtttgtat

SEQ ID NO: 226 - DNA, 505 bp, human
ggaagcacaatttccaccttattttttgaactttggcagtttcaatgtctgtctctgttgcttcggggcataagctgatcaccgtctagttgggaaagtaaccctacagggtttgtagggacatgatcagcatcctgatttgaaccctgaaatgttgtgtagacaccctcttgggtccaatgaggtagttggttgaagtagcaagatgttggcttttctggattttttttgccatgggttcttcactgaccttggactttggcatgattcttagtcatacttgaacttgtctcattccacctcttctcagagcaactcttcctttgggaaaagagttcttcagatcatagaccaaaaaagtcataccttcgaggtggtagcagtagattccaggaggagaagggtacttgctaggtatcctgggtcagtggcggtgcaaactggtttcctcagctgcctgtccttctgtgtgcttatgtctcttgtgacaattgttttcctccct

SEQ ID NO: 227 - DNA, 445 bp, human
agagaacccaccatggtgctgtctcctgccgacaagaccaacgtcaaggccgcctggggtaaggtcggcgcgcacgctggcgagtatggtgcggaggccctggagaggatgttcctgtccttccccaccaccaagacctacttcccgcacttcgacctgagccacggctctgcccaggttaagggccacggcaagaaggtggccgacgcgctgaccaacgccgtggcgcacgtggacgacatgcccaacgcgctgtccgccctgagcgacctgcacgcgcacaagcttcgggtggacccggtcaacttcaagctcctaagccactgcctgctggtgaccctggccgcccacctccccgccgagttcacccctgcggtgcacgcctccctggacaagttcctggcttctgtgagcaccgtgctgacctccaaataccgttaagctg

SEQ ID NO: 228 - DNA, 499 bp, human
tggaggaatttcacaagcacccgcccgtgcagtgggccttccaggagaccagtgtggagagcgccgtggacacgcccttcccagctggaatatttgtgaggctggaatttaagctgcagcagacaagctgccggaagagggactggaagaaacccgagtgcaaagtcaggcccaatgggaggaaacggaaatgcctggcctgcatcaaactgggctctgaggacaaagttctgggccggttggtccactgccccatagagacccaagttctgcgggaggctgaggagcaccaggagacccagtgcctcagggtgcagcgggctggtgaggacccccacagcttctacttccctggacagttcgccttctccaaggccctgccccgcagctaagccagcactgagctgcgtggtgcctccaggaccgctgccggtggtaaccagtggaagaccccagcccccagggagaggaccccgttctatccccagccatgataata

SEQ ID NO: 229 - DNA, 465 bp, human
tttgtcaggaaaccttggcctctgctaaggtgtatttggtccttgagaagtgggagcaccctacagggacactatcactcatgctggtggcattgtttacagctagaaagctgcactggtgctaatgccccttggggaaatggggctgtgaggaggaggattataacttaggcctagcctcttttaacagcctctgaaatttatcttttcttctatggggtctataaatgtatcttataataaaaaggaaggacaggaggaagacaggcaaatgtacttctcacccagtcttctacacagatggaatctctttggggctaagagaaaggttttattctatattgcttacctgatctcatgttaggcctaagaggctttctccaggaggattagcttggagttctctatactcaggtacctctttcagggttttctaaccctgacacggactgtgcatactttccc

SEQ ID NO: 230 - DNA, 526 bp, human
actggatgcgttaataacccatgttttaccttttgaaaaaataaatgaaggatttgacctgcttcactctgggaaaagtatccgtaccgtcctgacgttttgaggcaatagagatgccttcccctgtagcagtcttcagcctcctctaccctacaagatctggagcaacagctaggaaatatcattaattcagctcttcagagatgttatcaataaattacacatgggggctttccaaagaaatggaaattgatgggaaattatttttcaggaaaatttaaaattcaagtgagaagtaaataaagtgttgaacatcagctggggaattgaagccaacaaaccttccttcttaaccattctactgtgtcacctttgccattgaggaaaaatattcctgtgacttcttgcatttttggtatcttcataatctttagtcatcgaatcccagtggaggggacccttttacttgccctgaacatacacatgctgggccattgtgattgaagtcttctaactctgtctcagt

SEQ ID NO: 231 - DNA, 555 bp, human
gcagatttcttgcttcatatgacaaagcctcaattactaattgtaaaaactgaactattcccagaatcatgttcaaaaaatctgtaatttttgctgatcgaaagtgcttcattgactaaacagtattagtttgtggctataaatgattatttagatgatgactgaaaatgtgtataaagtaattaaaagtaatatggtggctttaagtgtagagatgggatggcaaatgctgtgaatgcagaatgtaaaattggtaactaagaaatggcacaaacaccttaagcaatatattttcctagtagatatatatatacacatacatatatacacatatacaaatgtatatttttgcaaaattgttttcaatctagaacttttctattaactaccatgtcttaaaatcaagtctataatcctagcattagtttaatattttgaatatgtaaacacctgtgttaatgctttgttaatgcttttcccactctcatttgttaatgctttcccactctcgggaaggatttgcattttgagctttatctctaaatgtgacatgca

SEQ ID NO: 232 - DNA, 453 bp, human
ggatgccgctctgtgacaaatgtggcagtggcatagtcggtgctgtggtgaaggcgcgggataagtaccggcaccctgagtgcttcgtgtgtgccgactgcaacctcaacctcaagcaaaagggctacttcttcatagaaggggagctgtactgcgaaacccacgcaagagcccgcacaaaacccccaagaggctatgacacggtcactctgtatcccaaagcttaagtctctgcaggcgtggcacacgcacgcacccacccacgcgcacttacacgagaagacattcatggctttgggcagaaggattgtgcagattgtcaactccaaatctaaagtcaaggctttagacctttatcctattgtttattgaggaaaaggaatgggaggcaaatgcctgctatgtgaaaaaaacatacacttagctatgttttgcaactctttttggggctag

SEQ ID NO: 233 - DNA, 396 bp, human
ttcctgaagctcttcggctgacagcccgctcggctcgccctctcccccggaggccgcgccctcccggaaaagccgggtcctccagaccccgaggccttgctctcagagcgggaggccccacccactggagagccccgcccctaaggtactatgagttctcaggggtcaagttcagaaacggcccagccagacctaaacccacacgcccacaaagtggattgcacacagacaagaactcccgtgcgggcctccactctattcccacccttgagggagcccccttactgggggagggtccttgcaattccagcgaatcggaggccaggccaggacgtccttgctccctgcaccctcactgttctgtgcactttttctacctacataaacacacgcatt

SEQ ID NO: 234 - DNA, 501 bp, human
gcagggctagttattccgatttcttgcacaattatttagctttttgtaagttcaacatgtaaattttaaagacataaatatagagagacttatgtgtttgaatataaatgatatatatggattagcatgtacctgtatattattaaacatgcaatgaactgactggtaagtgacgtctaattgtatggctagcaatgtaatttattcagactgtatttttgtacagagcagtgcactctaacctatgcctctgtgtcctctttaatgcctaaagctgtgcctagaaatttcatctgtcttaaaagtaaaatatacttcatgctgtttatgctattagtttctgtactgctattctatatttattatttttaaatatatgacatgtttactacttaaacatgaattcatggtatcctggttattttttttaagtcatctgggggaaaacctgtttatcactccagtgattttgagtttgcagtttcacaatcagttcttcat

SEQ ID NO: 235 - DNA, 94 bp, human
caggccttttccactttgagggaggtgcttcgaagaatgttgcccacacctaagtgttagaagcctatgtccgttcatccctgagaggtctgaa

SEQ ID NO: 236 - DNA, 508 bp, human
tggcggactccatgtttgtgatccctgcactccaagtagcacattttcagtgttcccgggcccctgtgtacttctacgagttccagcatcagcccagctggctcaagaacatcaggccaccgcacatgaaggcagaccatggtgatgagcttccttttgttttcagaagtttctttgggggcaactacattaaattcactgaggaagaggagcagctaagcaggaagatgatgaagtactgggccaactttgcgagaaatgggaacccgaatggcgagggtctgccacactggccgctgttcgaccaggaggagcaatacctgcagctgaacctacagcctgcggtgggccgggctctgaaggcccacaggctccagttctggaagaaggcgctgccccaaaagatccaggagctcgaggagcctgaagagagacacacagagctgtagctccctgtgccggggaggagggggtgggttcgctgacaggcgagggtcagcctgctgtg

SEQ ID NO: 237 - DNA, 475 bp, human
gagagctcgctttgagtgactgggttttgtgattgcctctgaagcctatgtatgccatggaggcactaacaaactctgaggtttccgaaatcagaagcgaaaaaatcagtgaataaaccatcatcttgccactaccccctcctgaagccacagcagggtttcaggttccaatcagaactgttggcaaggtgacatttccatgcataaatgcgatccacagaaggtcctggtggtatttgtaactttttgcaaggcatttttttatatatatttttgtgcacatttttttttacgtttctttagaaaacaaatgtatttcaaaatatatttatagtcgaacaattcatatatttgaagtggagccatatgaatgtcagtagtttatacttctctattatctcaaactactggcaatttgtaaagaaatatatatgatatataaatgtgattgcagcttttcaatgttagccacagt

SEQ ID NO: 238 - DNA, 491 bp, human
tgtggtttctgtagcaacacttctcatgaccatctgttttgtgtttatgatgattttttcaggtctgttggtcaatctcacaaccattgcatcttggctgtcatggcttcagtacttcagcattccacgatatggatttacggctttgcagcataatgaatttttgggacaaaacttctgcccaggactcaatgcaacaggaaacaatccttgtaactatgcaacatgtactggcgaagaatatttggtaaagcagggcatcgatctctcaccctggggcttgtggaagaatcacgtggccttggcttgtatgattgttattttcctcacaattgcctacctgaaattgttatttcttaaaaaatattcttaaatttccccttaattcagtatgatttatcctcacataaaaaagaagcactttgattgaagtattcaatcaagtttttttgttgttttctgttcccttgccatcacactgttgcacagca

SEQ ID NO: 239 - DNA, 400 bp, human
gaacacgcagagagtttccctagatatactcctgcctccaggtgctgggacacacctttgcaaaatgctgtgggaagcaggagctggggagctgtgttaagtcaaagtagaaaccctccagtgtttggtgttgtgtagagaataggacatagggtaaagaggccaagctgcctgtagttagtagagaagaatggatgtggttcttcttgtgtatttatttgtatcataaacacttggaacaacaaagaccataagcatcatttagcagttgtagccattttctagttaactcatgtaaacaagtaagagtaacataacagtattaccctttcactgttctcacaggacatgtacctaattatggtacttatttatgtagtcactgtatttctggattttt

SEQ ID NO: 240 - DNA, 467 bp, human
  misc_feature (84)..(85): n is a, c, g, or t
  misc_feature (136)..(137): n is a, c, g, or t
ctccagccccagagctgaaaacaccaagtgcctatttgagggtgtctgtctggagacttagagtttgtcatgtgtgtgtgtgtnnttggttaatgtgggtttatgggttttctttctttttttttttttttttttnnagtctacattagggggaagtgagcgcctcccatgtgcagacagtgtgtctttatagatttttctaaggctttccccaatgatgtcggtaatttctgatgtttctgaagttcccaggactcacacacccgttcccatctcacttgcccacccagtgtgacaaccctcggtgtggatatacccccgtggactcatggctcttccccacccccactttctataaatgtaggcctagaatacgcttctctgttgcaaaactcagctaagttcctgcttccaccttgatgttgaaatatcttatgtaagagggcaggggatgtcgtgaagatggc

SEQ ID NO: 241 - DNA, 352 bp, human
ggaatgacacttacttacgacccaactacagctgctatacagaacggattttatccttcaccatacagtattgctacaaaccgaatgatcactcaaacttctattacaccctatattgcatctcctgtatctgcctaccaggtgcaaagtccttcgtggatgcaacctcaaccatatattctacagcaccctggtgccgtgttaactccctcaatggagcacaccatgtcactacagcccgcatcaatgatcagccctctggcccagcagatgagtcatctgtcactaggcagcaccggaacatacatgcctgcaacgtcagctatgcaaggagcctacttgccacagtatg

SEQ ID NO: 242 - DNA, 520 bp, human
ggaacgaaaccagcgtcctattccagcgcctctacgggccccaggccctcctcttctccctcttctggcccaccttcctgctgaccggtggcctcctcattatcgccatggtgaagagcaaccagtacctgtccatcctggcggcccagaagtagagccatccatccatgccataccacttgtcagggcacaggggactggctgggcccccagggctgctccccacttgcagcacaatgccttctccacctgccctcccactcttccagtccaatccacgctgtcttctgttgcaggactaacctttgagaaatccttttgtgaagtcattgcctgctcaagaatgtacagtggctccccaatgccttggagccataaggccagccagttctagctctctattacctgtccccactcaactgactcatacctgtttccggctgcatcactatgtgccccacagagaacgatgatcgtcacctctgtgcctgagttctccctgttgtctcaaagcggtacc

SEQ ID NO: 243 - DNA, 538 bp, human
ccggtcagcaggatcatcgtgcacccacagttctacatcatccagactggagcggatatcgccctgctggagctggaggagcccgtgaacatctccagccgcgtccacacggtcatgctgccccctgcctcggagaccttccccccggggatgccgtgctgggtcactggctggggcgatgtggacaatgatgagcccctcccaccgccatttcccctgaagcaggtgaaggtccccataatggaaaaccacatttgtgacgcaaaataccaccttggcgcctacacgggagacgacgtccgcatcatccgtgacgacatgctgtgtgccgggaacacccggagggactcatgccagggcgactctggagggcccctggtgtgcaaggtgaatggcacctggctacaggcgggcgtggtcagctgggacgagggctgtgcccagcccaaccggcctggcatctacacccgtgtcacctactacttggactggatccaccactatgtccccaaaaagccgtgagtcaggcctggggtgt

SEQ ID NO: 244 - DNA, 489 bp, human
ggccaaatcaccgacctgaaggcggaaattcacgggggcagtctcattaatctgacttggacagctcctggggatgattatgaccatggaacagctcacaagtatatcattcgaataagtacaagtattcttgatctcagagacaagttcaatgaatctcttcaagtgaatactactgctctcatcccaaaggaagccaactctgaggaagtctttttgtttaaaccagaaaacattacttttgaaaatggcacagatcttttcattgctattcaggctgttgataaggtcgatctgaaatcagaaatatccaacattgcacgagtatctttgtttattcctccacagactccgccagagacacctagtcctgatgaaacgtctgctccttgtcctaatattcatatcaacagcaccattcctggcattcacattttaaaaattatgtggaagtggataggagaactgcagctgtcaatagcctagggc

SEQ ID NO: 245 - DNA, 461 bp, human
accccttcagcgactagagagctacaggagaatcaccagtggcaaatgtccccagaaagctgtgatcttcaagaccaaactggccaaggatatctgtgccgaccccaagaagaagtgggtgcaggattccatgaagtatctggaccaaaaatctccaactccaaagccataaataatcaccatttttgaaaccaaaccagagcctgagtgttgcctaatttgttttcccttcttacaatgcattctgaggtaacctcattatcagtccaaagggcatgggttttattatatatatatatattttttttttaaaaaaaaacgtattgcatttaatttattgaggctttaaaacttatcctccatgaatatcagttatttttaaactgtaaagctttgtgcagattctttaccccctgggagccccaattcgatcccctgtcacgtgtgggcaatgttccccc

SEQ ID NO: 246 - DNA, 534 bp, human
tgtgaagctttacgcgcacacggacaaaatgcccaaactggagcccttgcaaaaacacggcttgtggcattggcatacttgcccttacaggtggagtatcttcgtcacacatctaaatgagaaatcagtgacaacaagtctttgaaatggtgctatggatttaccattccttattatcactaatcatctaaacaactcactggaaatccaattaacaattttacaacataagatagaatggagacctgaataattctgtgtaatataaatggtttataactgcttttgtacctagctaggctgctattattactataatgagtaaatcataaagccttcatcactcccacatttttcttacggtcggagcatcagaacaagcgtctagactccttgggaccgtgagttcctagagcttggctgggtctaggctgttctgtgcctccaaggactgtctggcaatgacttgtattggccaccaactgtagatgtatatatggtgcccttctgatgctaagactccagaccttttgt

SEQ ID NO: 247 - DNA, 560 bp, human
tctactgcgtgacttgccatgagaccaagtttgccaagcattgcgtgaagtgcaacaaggccatcacatctggaggaatcacttaccaggatcagccctggcatgccgattgctttgtgtgtgttacctgctctaagaagctggctgggcagcgtttcaccgctgtggaggaccagtattactgcgtggattgctacaagaactttgtggccaagaagtgtgctggatgcaagaaccccatcactgggaaaaggactgtgtcaagagtgagccgcccagtctctaaagctaggaagcccccagtgtgccacgggaaacgcttgcctctcaccctgtttcccagcgccaacctccggggcaggcatccgggtggagagaggacttgtccctcgtgggtggtggttctttatagaaaaaatcgaagcttagcagctcctcgtggcccgggtttggtaaaggctccagtgtggtggcctatgaaggacaatcctggcacgactactgcttccactgcaaaaaatgctccgtgaatctggccaacaagcgctttgttttccacc

SEQ ID NO: 248 - DNA, 445 bp, human
gcataggagataaaacccccactgagatgctctcatgcctcagctggacccaccgtgtagacacacgacatgcaagagttgcagcggctgctccaactcactgctcaccctctctgtgagcagaaagaaccctactgacatgcatggttaacttcctcatcagaactctgcccttcccttctgttcttttgtgctttcaaataactaacacgaacttccagaaaattaacatttgaacttagctgtaattctaaactgacctttccccgtactaacgtttggtttccccgtgtggcatgcttttctgagccttcctactttaaagcatggaacatgcaggtgatttgggaagtgtagaaagacctgagaaaacgagcctgtttcagaggaacatcgtcacaacgaatacttctggaagcttaacaaaactaaccctgctgtcctt

SEQ ID NO: 249 - DNA, 185 bp, human
atttcgttttgcttttggttgcctgaatgttgtcaccaagtgaaaaaattatttaactatatgtaaaatttctcttttaaaaaaaagttttactgatgttaaacgttctcagtgccaatgtcagactgtgctcctccctctcctgaacctctaccctcaccctgagctgtcttgttgaaaacagt

SEQ ID NO: 250 - DNA, 551 bp, human
agccctttgctctggaggaagttctccagcttcagctcaactcacagcttctccaagcatcaccctgggagtttcctgagggttttctcataaatgagggctgcacattgcctgttctgcttcgaagtattcaataccgctcagtattttaaatgaagtgattctaagatttggtttgggatcaataggaaagcatatgcagccaaccaagatgcaaatgttttgaaatgatatgaccaaaattttaagtaggaaagtcacccaaacacttctgctttcacttaagtgtctggcccgcaatactgtaggaacaagcatgatcttgttactgtgatattttaaatatccacagtactcactttttccaaatgatcctagtaattgcctagaaatatctttctcttacctgttatttatcaatttttcccagtatttttatacggaaaaaattgtattgaaaacacttagtatgcagttgataagaggaatttggtataattatggtgggtgattattttttatactgtatgtgccaaagctttactactgtg

SEQ ID NO: 251 - DNA, 518 bp, human
gtgccatagtgcaggcttggggagctttaagcctcagttatataacccacgaaaaacagagcctcctagatgtaacattcctgatcaaggtacaattctttaaaattcactaatgattgaggtccatatttagtggtactctgaaattggtcactttcctattacacggagtgtgctaaaactaaaaagcattttgaaacatacagaatgttctattgtcattgggaaatttttctttctaacccagtggaggttagaaagaagttatattctggtagcaaattaactttacatcctttttcctacttgttatggttgtttggaccgataagtgtgcttaatcctgaggcaaagtagtgaatatgttttatatgttatgaagaaaagaattgttgtaagtttttgattctactcttatatgctggactgcattcacacatggcatgaaataagtcaggttctttacaaatggtattttgatagatactggattgtgtttgtgccatatttgtgccatt

SEQ ID NO: 252 - DNA, 419 bp, human
gtcaggactgttatggccctgttgtgctgagttagttggaacagaattcaagctccctgcactagtccacctgccccactgcttcttcgcttctctcttggaaagtccagtctctcctcggcttgcaatggaccccaactgctcctgcgccgctggtgtctcctgcacctgcgctggttcctgcaagtgcaaagagtgcaaatgcacctcctgcaagaagagctgctgctcctgctgccccgtgggctgtagcaagtgtgcccagggctgtgtttgcaaaggggcgtcagagaagtgcagctgctgcgactgatgccaggacaacctttctcccagatgtaaacagagagacatgtacaaacctggattttttttttataccaccttgacccatttgctacattccttttcctgtgaaa

SEQ ID NO: 253 - DNA, 506 bp, human
caggaaggaccttgctttggaccctacacacttcggctctctggacacttgcgacacctcaaggtgttctctgtagctcaatctgcaaacatgccaggcctcagggatcctctgctgggtgcctccttgccttgggaccatggccaccccagagccatccgatcgatggatgggatgcactctcagaccaagcagcaggaattcaaagctgcttgctgtaactgtgtgagattgtgaagtggtctgaattctggaatcacaaaccaagccatgctggtgggccattaatggttggaaaacactttcatccggggctttgccagagcgtgctttcaagtgtcctggaaagtctgctgcttctccaagctttcagacaagaatgtgcactctctgcttaggttttgcttgggaaactcaacttctttcctctggagacggggcatctccctctgatttccttctgctatgacaaaacctttaatctgcaccttacaactcggggacaa

SEQ ID NO: 254 - DNA, 565 bp, human
ttaccaatgacaaccctgagtgccgccttgtgaaagaaacccggatttgtgaggtgcggccttgtggacagccagtgtacagcagcctgaaaaagggcaagaaatgcagcaagaccaagaaatcccccgaaccagtcaggtttacttacgctggatgtttgagtgtgaagaaataccggcccaagtactgcggttcctgcgtggacggccgatgctgcacgccccagctgaccaggacatgtgaagatgcggttccgctgcgaagatggggagacattttccaagaacgtcatgatgatccagtcctcaaatgcaactacaactgcccgcatgccaatgaagcagcgtttcccttctacaggctgttcaatgacattcacaaatttagggactaaatgctacctgggtttccagggcacacctagacaaacaagggagaagagtgtcagaatcagaatcatggagaaaatgggcgggggtggtgtgggtgatgggactcattgtagaaaggaagccttgctcattcttgaggagcattaaggtatttcgaaactgccaagggtgc

SEQ ID NO: 255 - DNA, 413 bp, human
aaattgtggagttagcctcctgtggagttagcctcctgtggtaaaggaattgaagaaaatataacaccttacaccctttttcatcttgacattaaaagttctggctaactttggaatccattagagaaaaatccttgtcaccagattcattacaattcaaatcgaagagttgtgaactgttatcccattgaaaagaccgagccttgtatgtatgttatggatacataaaatgcacgcaagccattatctctccatgggaagctaagttataaaaataggtgcttggtgtacaaaactttttatatcaaaaggctttgcacatttctatatgagtgggtttactggtaaattatgttattttttacaactaattttgtactctcagaatgtttgtcatatgcttcttgcaatgc

SEQ ID NO: 256 - DNA, 253 bp, human
aaggcaagaggatgcatctttcttcctggtgtacaagcctttaaagacttctgctgctgatatgcctcttggatgcacactttgtgtgtacatagttacctttaactcagtggttatctaatagctctaaactcattaaaaaaactccaagccttccaccaaaacagtgccccacctgtatacatttttattaaaaaaatgtaatgcttatgtataaacatgtatgtaatatgctttctatgaatgatgtttg

SEQ ID NO: 257 - DNA, 445 bp, human
gaaggagacggtctggcggcttgaagaatttggacgatttgccagctttgaggctcaaggtgcattggccaacatagctgtggacaaagccaacttggaaatcatgacaaagcgctccaactatactccgatcaccaatgacaagttcaccccaccagtggtcaatgtcacgtggcttcgaaatggaaaacctgtcaccacaggagtgtcagagacagtcttcctgcccagggaagaccaccttttccgcaagttccactatctccccttcctgccctcaactgaggacgtttacgactgcagggtggagcactggggcttggatgagcctcttctcaagcactgggagtttgatgctccaagccctctcccagagactacagagaacgtggtgtgtgccctgggcctgactgtgggtctggtgggcatcattattgggaccatc

SEQ ID NO: 258 - DNA, 370 bp, human
aatattggatatcaactgcttgtaaaggtgctcctcctttttcttgtcattgctggtcaagattactaatatttgggaaggctttaaagacgcatgttatggtgctaatgtactttcacttttaaactctagatcagaattgttgacttgcattcagaacataaatgcacaaaatctgtacatgtctcccatcagaaagattcattggcatgccacaggggattctcctccttcatcctgtaaaggtcaacaataaaaaccaaattatggggctgcttttgtcacactagcatagagaatgtgttgaaatttaactttgtaagcttgtatgtggttgttgatcttttttttccttacagacacccataat

SEQ ID NO: 259 - DNA, 449 bp, human
atctcatacccgcaaattttaaccttgtcaacctctggggtattagtatgccctgacctgagtgaattcacccgtgacaaaactgacgtgaagattcaatggtacaaggattctcttcttttggataaagacaatgagaaatttctaagtgtgagggggaccactcacttactcgtacacgatgtggccctggaagatgctggctattaccgctgtgtcctgacatttgcccatgaaggccagcaatacaacatcactaggagtattgagctacgcatcaagaaaaaaaaagaagagaccattcctgtgatcatttcccccctcaagaccatatcagcttctctggggtcaagactgacaatcccgtgtaaggtgtttctgggaaccggcacacccttaaccaccatgctgtggtggacggccaatgacacccacatagagagcgccta

SEQ ID NO: 260 - DNA, 435 bp, human
agctcgaaagagtttgcaaccccatcatcagcaaactttaccaaggtggtcctggcggcggcagcggcggcggcggttcaggagcctccgggggacccaccatcgaagaagtggactaagcttgcactcaagtcagcgtaaacctctttgcctttctctctctctctttttttttgtttgtttctttgaaatgtccttgtgccaagtacgagatctattgttggaagtctttggtatatgcaaatgaaaggagaggtgcaacaacttagtttaattataaaagttccaaagtttgttttttaaaaacattattcgaggtttctctttaatgcattttgcgtgtttgctgacttgagcatttttgattagttcgtgcatggagatttgtttgagatgagaaaccttaagtttgcacacctgttctgtagaagcttg

SEQ ID NO: 261 - DNA, 476 bp, human
gctttttttgttgtcagctatcttaagaatcattaaatacacctgctttgggtaaaactctttgcaagcagtaattaacactagtaacagtgaaagcacaagatttccaaatcagtcgttttctcaaaaaaatatcgtataagtgactcatcctgtctgctaactccagacctcccagcttgaagccaaatctttccatgtgagattgatatggatttcctagaagtactggaatgttgtcatatcttgccctattttaattctgctatagaaaacaattgccttcacttttaaggagtaatttgaatattaataactctggtctagattttcatataatgtattaaagacaaagtagtgaacatcaatgaacatctgatagagataaactgtaatcaggcataagcttgtttgtatgttctggcagtgactaatcagtaaatgatgtcggtttgcccagtatcacttatcttctg

SEQ ID NO: 262 - DNA, 541 bp, human
cgtgaacggcaaagtggcgctggtgaccggcgcggctcagggcataggcagagcctttgcagaggcgctgctgcttaagggcgccaaggtagcgctggtggattggaatcttgaagcaggtgtacagtgtaaagctgccctggatgagcaatttgaacctcagaagactctgttcatccagtgcgatgtggctgaccagcaacaactgagagacacttttagaaaagttgtagaccactttggaagactggacattttggtcaataatgctggagtgaataatgagaaaaactgggaaaaaactctgcaaattaatttggtttctgttatcagtggaacctatcttggtttggattacatgagtaagcaaaatggaggtgaaggcggcatcattatcaatatgtcatctttagcagcccaccattgattgccaatggattgataacactcattgaagatgatgctttaaatggtgctattatgaagatcacaacttctaagggaattcattttcaagactatgatacaactccatttcaag

SEQ ID NO: 263 - DNA, 421 bp, human
gagtattacattggccttgggggacagaaaggaggaagttctgacttttcagggctaccttatttctactaaggacccagagcaggcctgtccatgccattccttcgcacagatgaaactgagctgggactggaaaggacagcccttgacctgggttctgggtataatttgcacttttgagactggtagctaaccatcttatgagtgccaatgtgtcatttagtaaaacttaaatagaaacaaggtccttcaaatgttcctttggccaaaagctgaagggagttactgagaaaatagttaacaattactgtcaggtgtcatcactgttcaaaaggtaagcacatttagaattttgttcttgacagttaactgactaatcttacttccacaaaatatgtgaatttgctgcttctgagaggca

SEQ ID NO: 264 - DNA, 281 bp, human
ggactggtgaagccttcggagaccctgtccctcacctgcagtgtctctggtggctccatcagtagttactactggagctggatccggcagcccccagggaagggactggagtggattgggtatatcaattacagtgggagcaccaactataacccctccctcaagagtcgagtcaccatatcagtagacacgtccaagaaccagttctccctgaagctgagctctgtgaccgctgcggacacggccgtgtattactgtgcgagaacgatttcggggggtcg

SEQ ID NO: 265 - DNA, 293 bp, human
aatagtgatgacgcagcttccagccaccctgtctgtgtctccaggggaaagagccaccctctcctgcagggccagtcagagtgttcgcagcaacttagcctggtaccagcaaaaacctggccaggctcccaggctcctcatctacggtgcatccaccagggccaatggtatcccagccaggttcagtggcagtgggtctgggacagagttcactctcaccatcagcggcctgcagtctgacgattttgcagtttattactgtcaacactataataactggcctccgtggacgt

SEQ ID NO: 266 - DNA, 284 bp, human
aattgtgttgacgcagtctccaggcaccctgtctttgtctccaggggaaagagccaccctctcctgcagggccagtcagagtattagcagcagtttcttagcctggtaccagcagaaagttggccaggctcccaggctcctcatctatggtgcatccagcagggccactggcatcccagacaggttcagtggcagtgggtctgggacagacttcactctcaccatcagcagactggagcctgaagattttgcagtgtattactgtcagcaatatggtagctcgc

SEQ ID NO: 267 - DNA, 263 bp, human
tcctccctgtctgcatctgtaggagacagagtcaccatcacttgccgggcaagtcagggcattagaaatgatttagcctggtatcagcagaaaccagggaaagcccctaagcgcctgatctatgctgcatccagtttgcaaagtggggtcccatcaaggttcagcggcagtggatctgggacagaattcactctcacaatcagcagcctgcagcctgaagattttgcaacttattactgtctacagcataatagttacccttg

SEQ ID NO: 268 - DNA, 458 bp, human
gcagttgagtcgtcatcacttttcagtgatgggagagtagatggtgaaatttattagttaatatatcccagaaattagaaaccttaatatgtggacgtaatctccacagtcaaagaaggatggcacctaaaccaccagtgcccaaagtctgtgtgatgaactttctcttcatactttttttcacagttggctggatgaaattttctagactttctgttggtgtatcccccccctgtatagttaggatagcatttttgatttatgcatggaaacctgaaaaaaagtttacaagtgtatatcagaaaagggaagttgtgccttttatagctattactgtctggttttaacaatttcctttatatttagtgaactacgcttgctcattttttcttacataattttttattcaagttattgtacagctgtttaagatgggcagctagttcgtagctttccca

SEQ ID NO: 269 - DNA, 494 bp, human
tgaggagaagtctgccgttactgccctgtggggcaaggtgaacgtggatgaagttggtggtgaggccctgggcaggctgctggtggtctacccttggacccagaggttctttgagtcctttggggatctgtccactcctgatgctgttatgggcaaccctaaggtgaaggctcatggcaagaaagtgctcggtgcctttagtgatggcctggctcacctggacaacctcaagggcacctttgccacactgagtgagctgcactgtgacaagctgcacgtggatcctgagaacttcaggctcctgggcaacgtgctggtctgtgtgccggcccatcactttggcaaagaattcacccaaccagtgcaggctgcctatcagaaagtggtggctggtgtggctaatgccctggcccacaagtatcactaagctcgctttcttgctgtccaatttctattaaaggttcctttgttccctaagtccaactactaaactg

SEQ ID NO: 270 - DNA, 445 bp, human
agagaacccaccatggtgctgtctcctgacgacaagaccaacgtcaaggccgcctggggtaaggtcggcgcgcacgctggcgagtatggtgcggaggccctggagaggatgttcctgtccttccccaccaccaagacctacttcccgcacttcgacctgagccacggctctgcccaggttaagggccacggcaagaaggtggccgacgcgctgaccaacgccgtggcgcacgtggacgacatgcccaacgcgctgtccgccctgagcgacctgcacgcgcacaagcttcgggtggacccggtcaacttcaagctcctaagccactgcctgctggtgaccctggccgcccacctccccgccgagttcacccctgcggtgcacgcctccctggacaagttcctggcttctgtgagcaccgtgctgacctccaaataccgttaagctg

SEQ ID NO: 271 - DNA, 485 bp, human
gggagtttcctgagggttttctcataaatgagggctgcacattgcctgttctgcttcgaagtattcaataccgctcagtattttaaatgaagtgattctaagatttggtttgggatcaataggaaagcatatgcagccaaccaagatgcaaatgttttgaaatgatatgaccaaaattttaagtaggaaagtcacccaaacacttctgctttcacttaagtgtctggcccgcaatactgtaggaacaagcatgatcttgttactgtgatattttaaatatccacagtactcactttttccaaatgatcctagtaattgcctagaaatatctttctcttacctgttatttatcaatttttcccagtatttttatacggaaaaaattgtattgaaaacacttagtatgcagttgataagaggaatttggtataattatggtgggtgattattttttatactgtatgtgccaaagctttactactgtg

SEQ ID NO: 272 - DNA, 435 bp, human
tcctgccgacaagaccaacgtcaaggccgcctggggtaaggtcggcgcgcacgctggcgagtatggtgcggaggccctggagaggatgttcctgtccttccccaccaccaagacctacttcccgcacttcgacctgagccacggctctgcccaggttaagggccacggcaagaaggtggccgacgcgctgaccaacgccgtggcgcacgtggacgacatgcccaacgcgctgtccgccctgagcgacctgcacgcgcacaagcttcgggtggacccggtcaacttcaagctcctaagccactgcctgctggtgaccctggccgcccacctccccgccgagttcacccctgcggtgcacgcctccctggacaagttcctggcttctgtgagcaccgtgctgacctccaaataccgttaagctggagcctcggtggc

SEQ ID NO: 273 - DNA, 583 bp, human
tacgcagactccgtgaagggccgattcaccatctccagagacaattccaagaacacgctgtatcttcaaatgaacagcctgagagccgaggacacggccgtattactgtgcaagagagacttgtgtgattggggccaaggtaccctggtcaccgtgtcgagaggtggcggtggctcgggcggtggtgggtcgggtggcggcggatcttctgagctgactcaggaccctgctgtgtctgtggccttgggacagacagtcaggatcacatgccaaggagacagcctcagaagctattatgcaagctggtaccagcagaagccaggacaggcccctgtacttgtcatctatggtaaaaacaaccggccctcagggatcccagaccgattctctggctccagctcaggaaacacagcttccttgaccatcactggggctcaggcggaagatgaggctgactattactgtaactcccgggacagcagtggtaaccatgtggtattcggcggagggaccaagctgaccgtcctaggtgcggccgcagaacaaaaactcatctcagaagaggatctgaatggggccgcat

SEQ ID NO: 274 - DNA, 484 bp, human
ttgcacaagtttcctgggctggaccgtttcaacagagaggcttatttgactttatgctagaagatgaggcttctgggataggcccagaagttcctgatgaccgcgacttcgagccctccctaggcccagtgtgccccttccgctgtcaatgccatcttcgagtggtccagtgttctgatttgggtctggacaaagtgccaaaggatcttccccctgacacaactctgctagacctgcaaaacaacaaaataaccgaaatcaaagatggagactttaagaacctgaagaaccttcacgttgtctaccttcataacaacaatatctctgtagttggatcaagtgacttctgcccacctggacacaacaccaaaaaggcttcttattcgggtgtgagtcttttcagcaacccggtccagtactgggagatacagccatccaccttcagatgtgtctacgtgcgctctgccattcaactcggaaacta

SEQ ID NO: 275 - DNA, 502 bp, human
tgtccatacagagtgtgcattccctggcaggggctcctgctcacagcctcgcttttaaccttctggaacctgccaaacagtgcccagaccaatattgatggtgtgccgttcaatgtcgcagaagggaaggaggtccttctagtagtccataatgagtcccagaatctttatggctacaactggtacaaagggcaaagggtgcatgccaactatcgaattataggatatgtaaaaaatataagtcaagaaaatgccccagggcccgcacacaacggtcgagagacaatataccccaatggaaccctgctgatccagaacgtcacccacaatgacgcaggaatctataccctacacgttataaaagaaaatcttgtgaatgaagaagtaaccagacaattctacgtattctatgagtcagtacaagcaagttcacctgacctctcagctgggaccgctgtcagcatcatgattggagtactggctgggatggctctgatatagc

SEQ ID NO: 276 - DNA, 510 bp, human
tgcacagtactcctggcttatcaatggaacattccagcaaagcacacaagagctctttatccctaacatcactgtgaataatagtggatcctatacctgccacgccaataactcagtcactggctgcaacaggaccacagtcaagacgatcatagtcactgagagacagaatctcaccatgttgcccgggctggactcgaactcctgggctcaagcaatcctcccatctgtttcccaaagtgctgagattacagataatgctctaccacaagaaaatggcctctcacctggggccattgctggcattgtgattggagtagtggccctggttgctctgatagcagtagccctggcatgttttctgcatttcgggaagaccggcagggcaagcgaccagcgtgatctcacagagcacaaaccctcagtgtccaaccacactcaggaccactccaatgacccacctaacaagatgaatgaagttacttattctaccctgaactttgaagccca

SEQ ID NO: 277 - DNA, 244 bp, human
tcctccttcccttacggaattacatcttgatggcaacaaaatcagcagagttgatgcagctagcctgaaaggactgaataatttggctaagttgggattgagtttcaacagcatctctgctgttgacaatggctctctggccaacacgcctcatctgagggagcttcacttggacaacaacaagcttaccagagtacctggtgggctggcagagcataagtacatccaggttgtctaccttcat

SEQ ID NO: 278 - DNA, 198 bp, human
tcacaccgtagcccacatggatagcacagttgtcagacaagattccttcagattccgagttgcctaccggttgttttcgttgttgttgttgttgtttttctttttctttttttttttgaagacagcaataaccacagtacatattactgtagttctctatagttttacatacattcataccataactctgttctctcc

SEQ ID NO: 279 - DNA, 430 bp, human
  misc_feature (42)..(42): n is a, c, g, or t
  misc_feature (93)..(94): n is a, c, g, or t
  misc_feature (107)..(107): n is a, c, g, or t
  misc_feature (121)..(121): n is a, c, g, or t
  misc_feature (125)..(125): n is a, c, g, or t
  misc_feature (144)..(144): n is a, c, g, or t
  misc_feature (155)..(155): n is a, c, g, or t
  misc_feature (163)..(163): n is a, c, g, or t
  misc_feature (167)..(167): n is a, c, g, or t
  misc_feature (174)..(174): n is a, c, g, or t
  misc_feature (184)..(184): n is a, c, g, or t
  misc_feature (188)..(188): n is a, c, g, or t
  misc_feature (193)..(193): n is a, c, g, or t
  misc_feature (199)..(199): n is a, c, g, or t
  misc_feature (254)..(254): n is a, c, g, or t
  misc_feature (270)..(270): n is a, c, g, or t
  misc_feature (361)..(361): n is a, c, g, or t
  misc_feature (364)..(364): n is a, c, g, or t
  misc_feature (377)..(377): n is a, c, g, or t
cactttccaatgccacagacaactcacattgttcaactcccnttctcggggtgggacagacgagacaacagcacacaggcagccagccgtggnnccagaggctcganggggctcaggggcntcanggcacccgtccccacacgnagggccccgtngggtgggncctnggccctngctttctacngccnaatgnttatgnccagctccatgttctcccaaataccgttgatgtgaattattttaaaggcaaaacngtgctctttattttanaaaacactgataatcacactgcggtaggtcattcttttgccacatccctatagaccactgggtttggcaaaactcaggcagaagtggaganccnttctagacatcantgtcagccttgctacttgaaggtacaccccatagggtcggaggtgctgtcccc

SEQ ID NO: 280 - DNA, 135 bp, human
gaggcaaatggatctcgatatttcagatgggcttttgatgcactgttgccaaggaaggctttttctgattttttgacaaatgaatttttgcacactttcattggtgtctttcggcaacttacacacattgaaaat

SEQ ID NO: 281 - DNA, 260 bp, human
  misc_feature (45)..(45): n is a, c, g, or t
  misc_feature (48)..(48): n is a, c, g, or t
gaggacttaggagagatctgaactccagctgccctacaaactccntcncagcttttcttctcacttcatgtgaaaactactccagtggctgactgaattgctgacccttcaagctctgtccttatccattacctcaaagcagtcattccttagtaaagtttccaacaaatagaaattaatgacactttggtagcactaatatggagattatcctttcattgagccttttatcctctgttctcctttgaagaacccctcac

SEQ ID NO: 282 - DNA, 131 bp, human
  misc_feature (85)..(85): n is a, c, g, or t
gcactgggaggcccaagagccaatccagatgcctgagacaacggagactgtgctctgtgccctgggcctggtgctgggcctagtnggcatcatcgtgggcaccgtcctcatcataaagtctctgcgttctg

SEQ ID NO: 283 - DNA, 466 bp, human
tgcagaagtcaaaccctcatgacaaagtaggcacaagtctacaataagctaaatcagaatttacaaatacaagtgtcccaggtagcattgactcccgtcattggagtgaaatggatcaaagtttgaattaaggcctatggtaaggtaacattgctttgttgtacttttgaacaagagctcctcctgatcactattacatatttttctagaaaatctaaagttcagaagagaatgtatcactgctgacttttattccaatatttggatggagtaagttttagggtagaattttgttcagtttggatttaatcttttgaaaagtaaattccttgtttactggtttgactataattctctgttatctttacgaggtaaaactgcaagctgactagcatgttctgtgaatctgccattcctaaaaattttataaacacttgatacttttcactgataatggatcgctcca

SEQ ID NO: 284 - DNA, 496 bp, human
  misc_feature (304)..(304): n is a, c, g, or t
  misc_feature (307)..(307): n is a, c, g, or t
tcacagcgggcaggacggaccccgcccacgacgtgcgggtggcggtggtgcagtacagcggcacgggccagcagcgcccagagcgggcgtcgctgcagttcctgcagaactacacggccctggccagtgccgtcgatgccatggactttatcaacgacgccaccgacgtcaacgatgccctgggctatgtgacccgcttctaccgcgaggcctcgtccggcgctgccaagaagaggctgctgctcttctcagatggcaactcgcagggcgccacgcccgctgccatcgagaaggccgtgcaggnagnccagcgggcaggcatcgagatcttcgtggtggtcgtgggccgccaggtgaatgagccccacatccgcgtcctggtcaccggcaagacggccgagtacgacgtggcctacggcgagagccacctgttccgtgtccccagctaccaggccctgctccgcggtgtcttccaccagacagtctccaggaaggt

SEQ ID NO: 285 - DNA, 541 bp, human
  misc_feature (54)..(54): n is a, c, g, or t
  misc_feature (91)..(91): n is a, c, g, or t
  misc_feature (99)..(99): n is a, c, g, or t
  misc_feature (106)..(106): n is a, c, g, or t
  misc_feature (110)..(110): n is a, c, g, or t
  misc_feature (136)..(136): n is a, c, g, or t
  misc_feature (151)..(151): n is a, c, g, or t
gaatttcacctgtaaacctgagtcgtacagaaagctgcctggtatatccaaaangctttttattcctcctgctcatattgtgattctgccntttggggnacttttncttnaaaccttcagttatgatttttttttncatacacttattggnaactctgcttgatttttgcctcttccagtcttcctgacactttaattaccaacctgttacctactttgactttttgcatttaaaacagacactggcatggatatagttttacttttaaactgtgtacataactgaaaatgtgctatactgcatactttttaaatgtaaagatatttttatctttatatgaagaaaatcacttaggaaatggctttgtgattcaatctgtaaactgtgtattccaagacatgtctgttctacatagatgcttagtccctcatgcaaatcaattactggtccaaaagattgctgaaattttatatgcttactgatatattttacaattttttatcatgcatgtcctgtaaaggttacaagcctgcacaataa

SEQ ID NO: 286 - DNA, 557 bp, human
tcttccccttgagcttcagagaggagagttggcatggttaaatctgaatggttacctcactgctgaaaacccagaggggcgtggcacactcgcttgtgtggaaaagcctctaaatgcatcccttcctttctttcctgcttcctttgccttacaattgaagcagcccgtggtaccatcacagtatgcagagacttcctcacctttcatatctagggaccacccccgatgcattggtgagggtgggcacttataaatgcctgctattgttaagccattccagcctcttcctctgaatagaccagacgccctttcacttagttcagtgccagtccttttgccttcccaaccctgctgttaggcctgctgttccctttgctcttgattaggagagatggaaggagatgagctcccataactgaattggcctttggttcatgttttctccccatatgtatatatgccatatgtgaatatgccatatatatgtgccaacaaatctatctacgttgttcttttcaaattagcacgcagataggaattttgagtttcttcttctt

SEQ ID NO: 287 - DNA, 418 bp, human
tatgtaaactttaacttccactttgtataaatttttaagtgtcagactatccattttacacttgctttatttttcattacctgtagctttgggcagatttgcaacagcaaattaatgtgtaaaattggattattactacaaaaccgtttagtcatatctatctaatcagatcttcttttgggaggatttgatgtaagttactgacaagcctcagcaaacccaaagatgttaacagtattttaagaagttgctgcagattcctttggccactgtatttgttaatttcttgcaatttgaaggtacgagtagaggtttaaagaaaaatcagtttttgttcttaaaaatgcatttaagttgtaaacgtctttttaagcctttgaagtgcctctgattctatgtaacttgttgcagactggtg

SEQ ID NO: 288 - DNA, 199 bp, human
cgccggctcctgcaaatgcaaagagtgcaaatgcacctcctgcaagaaaagctgctgctcctgctgccctgtgggctgtgccaagtgtgcccagggctgcatctgcaaaggggcgtcggacaagtgcagctgctgcgcctgatgctgggacagccccgctcccagatgtaaagaacgcgacttccacaaacctggattt

SEQ ID NO: 289 - DNA, 500 bp, human
caactgtttttgcgactttataggcaggtaaattttgctattactattgaatacaaatgacaattcatttatgaccactcaaacagcgttagtaaccatttagtgacaaaggattaaaacatccatctggatgttaattttgaagatgtaaattatatgttgtttaaatttttccaggcatctgaaaaccttatctgctagacaatgtaagattcacacagagttatctgggattctgattttttaaatagtacatatcattaaaccattttctctaaatgtaagaagagcagaaaaaatcttataagattatcagatttttctaatgacacagaaatgtaagaaaaaaatccctttatattgaaaaaagatgcagtcaaagtcttttcagacatgcccaaactttgagaatttcttcaaccatctaatgctataaagatttttgttcttcctgttcacaaccagttgtataacagaaatactagctactgttttccttc

SEQ ID NO: 290 - DNA, 546 bp, human
gatgggtcgtgtgatgagatgcatttaaggccgatagtgatagatgttttttttatttcttgaacacaggctttgtctgaatgatgttcttttatctcttgaacacaagctttgaatgataactacaggttttaagtgctgttacattaataccataatgtgatgtgttagaaacaaagggatatttcaaaggtagatatttgaaaattctctagtctcaatatgtatgtgtattgaatatactctaaaaataaatgtgcaatttgctagtaggacaatgcagtgactgactagcattaggtatgtttcttttatatcctagctatgtcccactttcttctaagtgcaatcctttcatgttcacttgctgttttaccccatctactctaacttcatttggaaggcttgtctagagtatagcatgtatttttacctttgcagtgaattgcatgtgctaattgtaaccacagctatttttatgttgacataactccaaatgttatattaaatgttctattatatattagctctaatcccttaagtaaa

SEQ ID NO: 291 - DNA, 419 bp, human
  misc_feature (249)..(249): n is a, c, g, or t
acagtgttctctaatgttacagatgagatgcgcattgccaaagaggagatttttggaccagtgcagcaaatcatgaagtttaaatctttagatgacgtgatcaaaagagcaaacaatactttctatggcttatcagcaggagtgtttaccaaagacattgataaagccataacaatctcctctgctctgcaggcaggaacagtgtgggtgaattgctatggcgtggtaagtgcccagtgcccctttggntgggattcaagatgtctggaaatggaagagaactgggagagtacggtttccatgaatatacagaggtcaaaacagtcacagtgaaaatctctcagaagaactcataaagaaaatacaagagtggagagaagctcttcaatagctaagcatctccttacagtcactaatat

SEQ ID NO: 292 - DNA, 538 bp, human
caaggtcccactctaggtgatagacagggaccccttctactgaacctttgaggaaaggaggaaggaagaaatgcgtttagatcttggatgcagacctttcaaagggttaaatgtaaccatatggatcaaccacatgcacatccttactacagaatccgtcctttcatttcaacttatagcaagctatgatttttatatataaatattatataaataatgtataaaacattaaaagttaactatgtaagatattatttctgaaacaatttagctatatccactatgattataaactgtgtctcgacctgtgttatttacattagctgcttaaaaaagcattgagttaatttttttaaatatcaactaaaatatcatagttctgtggtagacattgttttataatgaaataactgcaactagagaaaactgtataaaaacattaaattgtcagtatttttgtaaggttccattttgtaaagagaataatattcaaagacttttgtagcatacaaagtgaaaacttgtatctgcgaaacta

SEQ ID NO: 293 - DNA, 431 bp, human
  misc_feature (131)..(131): n is a, c, g, or t
aagcagcctagggatgagcatttctttgaaagcaattaggttattcacctggtattaaaactatttactgttaaaaaatctgtgacttcatgaagttgatttttaaaggcagcatcaaaaactgaaaaggnaagggaaaaaataggcagcttctctgcacttgtttggagctccccaaaacaggagccatggagaagtggcatcaagaccgggctgccctttcgagaacaccctgtggcagttcagagacacgcttttcctacactgcatgcagcccctctttccagcactggaaagaagtggtcttgagcccagctgagaagcacttcacactcctctctcttgttctgaatggtgtttgtgtcagtctgcagctgtgtatggtattatgtcttataatcctgcatcacttctatcctatccagtcatat

SEQ ID NO: 294 - DNA, 445 bp, human
gggtagatgggagtgtcgcttgtatgttatcgtacagctgacatgtatttttgtctattctttattatcttagtttcatgctatgtatgtaccataaccaacctattgcctatgagaaacatgtaagataatgtatttacagccattgttacaagtttataatgtatttttctatcttgttttatatgtatgttatataacattcaaaaagaatttttttcttgattgagaaaaggatacaaaatgcaaaatccacaattttgataactgaaaattgccaattgttttgcagtactttattatattgggtgtcttgtcttttttgggcttttagttagctaatgaatgaatacattagacacttttgggttttagttgggattttacatagcttgcattttaattctttggttctttgctgtttctattaacccacagcattatt

SEQ ID NO: 295 - DNA, 394 bp, human
gaaggttgtcatgggctgttccttgggggtttttatcctgctcaccgtggagataagcctgcggcttgtctaaccagcgcagcgcaaaggtctcaatgccttttggtaacatccgtcattgcagaagaaagtttacacgacgtcaaaaagtgacgttcatgctaagtgtttttccagaaatattggtttcatgtttcttattggctctgcctcctgtgcttatatcatccaaaaactttttaaaaaggtccagaattctattttaacctgatgttgagcacctttaaaacgttcgtatgtgtgttgcactaattctaaactttggaggcattttgctgtgtgaggccgatcgccactgtaaaggtcctagagttgcctgtttgtctctggagat

SEQ ID NO: 296 - DNA, 179 bp, human
gagatttaccatgtatcagtgcctggctttttgttataaagctttgtttgtctagtgctcttttgctataaaatagactgtagtacaccctagtaggaaaaaaaaaaaactaaatttaaaaataaaaaatatatttggcttatttttcgcaggagcaatccttttataccatgaatatt

SEQ ID NO: 297 - DNA, 540 bp, human
  misc_feature (59)..(59): n is a, c, g, or t
  misc_feature (103)..(103): n is a, c, g, or t
gtttatacaactgtcagtccacccagtggtgcaactggttctgattcagtcttccgatncctttttatttttcactttttcctatttctgaattttttttttnatttgtgatcttgattttgatgaggggttggggagtggggagggagtcgaaccaagacttggagttaagaggattttcatcttttgcatccaacaggcagaatatgatctgtgtccaaaagtgaacttgagtcaggaatgaatcaatttcagcataaacaagcacaaaaatttagtctgctggctgactggaagcaaaaaagtcaagatggaatatgatgaattccaacacaatggggcaccaaggcctttaggcctctctttttattttgctttggttttgtttgtttttctttagagacatgctctttctcatgggacttgaagtggactcatctttgtgcagtgctggttttgccatactcatttcaagtattatagacatatgtaatggtgaaaatatatgaactgtggcctttttcattcttgttacttgtg

SEQ ID NO: 298 - DNA, 480 bp, human
gaagtgactgttgtaccatggttgtgcacatgcttcagaatcctatggaagagaatattcctacttgcagtacatcaaaggaatggatggtggaccctactattcatgttttgagacataaatgttcactttaaagcaattgcataatagataaaaacctgaactttcattggatttttgttaattttcctcattttgaattatgtgcactaccatagctacatcagtttgatacagtattgaaaaattatcagttatattttgctgtttatgatctatttgtagattaggattaaaatggatttaatccatttttaaggctgtgtgaatttttctaaacaagaaccatttgcaatatggatttcttagagattaaaccaattataacttattagcagtcgcgagcacatgttcatatagtcaatgtaaaaatacactaatgagtatttggtaaatcccagtaggcttttaccattagca

SEQ ID NO: 299 - DNA, 124 bp, human
tccctcacttctcttaagccaagaagtttgctttccctagctgcagtgtagatggctcttgtttttgtttttttgttttaatcatttggcattcacatgtggctgttaatatgtgcttgttttt

SEQ ID NO: 300 - DNA, 547 bp, human
  misc_feature (59)..(59): n is a, c, g, or t
  misc_feature (308)..(308): n is a, c, g, or t
tggtgggagggctctcaatcagcagggccccaggagggaagaagaagtggggcaaagcntggcctcgccgctcgggagctttgccatctgagccacgcctcctccaggccatgctccttgaacttggaaatgtcaaccggagcccttacaccagccctccagcatctaatagacttgaatctactctaaacgaatatttaatccaacctcactacattgtagctcagtccaacgactaaccctgaaatgggggtgttccagccttcagcgagatggccaagcggtcccctgggggctgtggcagcggncttatccttctctgttgccaaccttgccgtccgacctcctccgcccccatgcggtgaccccgtccgtgtctgtgtctgtccatacgtgtgagtccagctaaaaagacaaaacagaacccgtgggcccagctcggaaggtgcgtggagaaggctccgacgtctccgaagtgcagcccttgggatggcattccgttgtgtgccttattcctggagaatctgtatacggctcgcctatagaa

SEQ ID NO: 301 - DNA, 350 bp, human
  misc_feature (90)..(90): n is a, c, g, or t
  misc_feature (124)..(124): n is a, c, g, or t
  misc_feature (143)..(143): n is a, c, g, or t
  misc_feature (150)..(150): n is a, c, g, or t
  misc_feature (157)..(158): n is a, c, g, or t
  misc_feature (162)..(163): n is a, c, g, or t
  misc_feature (168)..(168): n is a, c, g, or t
  misc_feature (223)..(223): n is a, c, g, or t
cttctccaagcatcaccctgggagtttcctgagggttttctcataaatgagggctgcacattgcctgttctgcttcgaagtattcaatanccgctcagtattttaaatgaagtgattctaagantttggtttgggatcaatanggaaagncatatgnncagnnccaanccaagatgcaaatgttttgaaatgatatgaccaaaattttaagtaggaaagtcancccaaacacttctgctttcacttaagtgtctggcccgcaatactgtaggaacaagcatgatcttgttactgtgatattttaaatatccacagtactcactttttccaaatgatcctagtaattgcct

SEQ ID NO: 302 - DNA, 414 bp, human
  misc_feature (157)..(157): n is a, c, g, or t
gtatcaaaatcttccaattatcatgctcacctgaaagaggtatgctctcttaggaatacagtttctagcattaaacaaataaacaaggggagaaaataaaactcaaggagtgaaaatcaggaggtgtaataaaatgttcctcgcattcccccccgcntttttttttttttttgactttgccttggagagccagagcttccgcattttctttactattctttttaaaaaaagtttcactgtgtagagaacatatatgcataaacataggtcaattatatgtctccattagaaaaataataattggaaaacatgttctagaactagttacaaaaataatttaaggtgaaatctctaatatttataaaagtagcaaaataaatgcataattaaaatatatttggacataacagactt

SEQ ID NO: 303 - DNA, 426 bp, human
  misc_feature (324)..(324): n is a, c, g, or t
caggaggacacagcattattctagtgctgtactgttccgtacggcagccactacccacatgtaactttttaagatttaaatttaaattagttaacattcaaaacgcagctccccaatcacactagcaacatttcaagtgcttgagagccatgcatgattagtggttaccctattgaataggtcagaagtagaatcttttcatcatcacagaaagttctattggacagtgctcttctagatcatcataagactacagagcacttttcaaagctcatgcatgttcatcatgttagtgtcgtattttgagctggggttttgagactncccttagagatagagaaacagacccaagaaatgtgctcaattgcaatgggccacatacctagatctccagatgtcatttcccctctcttattttaagttatg

SEQ ID NO: 304 - DNA, 295 bp, human
  misc_feature (114)..(114): n is a, c, g, or t
  misc_feature (224)..(224): n is a, c, g, or t
accaatgaggttcctgaggtcacagtgttttccaagtctcccgtgacactgggtcagcccaacaccctcatctgtcttgtggacaacatctttcctcctgtggtcaacatcacntggctgagcaatgggcactcagtcacagaaggtgtttctgagaccagcttcctctccaagagtgatcattccttcttcaagatcagttacctcaccttcctcccttctgntgatgagatttatgactgcaaggtggagcactggggcctggatgagcctcttctgaaacactgggagcctg

SEQ ID NO: 305 - DNA, 537 bp, human
  misc_feature (368)..(368): n is a, c, g, or t
cacatggttctagcctggacctggctgggctccatgagaatgagttgcctccaccctgtcccaacagctgacagccaggagccactctcccagctgcaggcctttgtggtccatcttgtcctgcttcctcactgtggacccctgtctgggccaccctagtgtgctaagctgagcagtgcagtgtgaacagggcccatggtgtattctaggccacagcccagcactcctctgggctgctctcaaaccatgtcccatcttcagcatccctcccaccaacttactcccctgtggtgagtaccgtggaaccccagcccacctcactatcatactcagcttcccctgatggcccatcccagcccctgaagctntatgccaagaacacagctaccgcacaccaccctgaaacagccacagccaaggtaggcatgcatatgaggtcttccccataccctctgggtgttgagaggtttagccacatgagggagcagaggacaatctctgcagggctgggagtgggtagggactgaaggtctcaat

SEQ ID NO: 306 - DNA, 466 bp, human
tcagtgtctgggctcatgacttgtaaatggaagctgatgtgaacaggtaattaatattatgacccacttctatttactttgggaaatatcttggatcttaattatcatctgcaagtttcaagaagtattctgccaaaagtatttacaagtatggactcatgagctattgttggttgctaaatgtgaatcacgcgggagtgagtgtgcccttcacactgtgacattgtgacattgtgacaagctccatgtcctttaaaatcagtcactctgcacacaagagaaatcaacttcgtggttggatggggccggaacacaaccagtctttttgtatttattgttactgagacaaaacagtactcactgagtgtttttcagtttcctactggtggttttgatattgtttgtttaagatgtatatttagaatgacatcatctaagaagctgattttgctaaactcctgttccc

SEQ ID NO: 307 - DNA, 474 bp, human
gaactcatgtgatttacccttttcaactttttggaaaacgatttaatttattctaattagattaaccctattaatctatggattgggtatcaaaatgaatgccagtccagatgtgcctagacacgaaattggagctgaggactctcacgatatgcaagttcatccaacgtgaagataccataagctttttctctgaaccagagaaatgaaagtcagtttaagaggctgatagatcttggccctgttaaggcatccacttcacagttctgaaggctgagtcagccccactccacagttaggccaagaattagattttaaaacttcatctgtctgtcccagttaactgttaaataaggcctcatcctccactgaagagtatggattgaaggattgtgaactatgtttagtgtgattgtgaacttggtgcctaatgttccatgtctgaagtttgccccagtgctacacgttggagta

SEQ ID NO: 308 - DNA, 388 bp, human
  misc_feature (218)..(218): n is a, c, g, or t
gatgcaattggttctcctgcattgagatttgatttaacagtgttatgttaacatttatacttgccttggactgtagaacagaacttaaatgggaatgtattagttttacaactacaatcaagtcattttacctttacccagtttttaatataaaacttaaattttgaaattcactgtgtgactaatagcatgatgctctgcagttttattaagaaatnagcctaaccatacaactctcatttccttagtaagccaaattaggattaacttctataaacagtgttgggaacaatgtttaacattttgtgccaatttgttcctgtattcatgtatgtaagttacagatctgactcttcatttttaagttccttgttacatcatggtcatt

SEQ ID NO: 309 - DNA, 379 bp, human
cctatcctacttgcccatatgagcacggctccccatggccacatactcctgcaaagcttttatgctgcttcgcttttctctaaacagatctgatattgctgctcctgtggttttctcaaaattaactttgccgtggtttttaaaaaggaatcaaaatgcattgttgcattaagctttttcaataaaggaaaattacggaaggaaaataggcaacaccagcaaattatatgtggacaggttctaaactctatatatacatatatatatatatatctatatatctatatacgtaatcatctagttctgtcatcttactgaaaggaataacacttctaaagatcaccatttctgagaagttcttggaaatctttatgtctaa

SEQ ID NO: 310 - DNA, 519 bp, human
  misc_feature (130)..(130): n is a, c, g, or t
  misc_feature (144)..(144): n is a, c, g, or t
  misc_feature (167)..(167): n is a, c, g, or t
  misc_feature (169)..(169): n is a, c, g, or t
  misc_feature (268)..(268): n is a, c, g, or t
  misc_feature (349)..(349): n is a, c, g, or t
  misc_feature (358)..(358): n is a, c, g, or t
  misc_feature (478)..(478): n is a, c, g, or t
caccgcgcagagctcagggggtggtgcgcccggcccttctgcggcgcacagcccagcccaggaacgcgggcggtgcggactcagcgggccgggtgcaggcgcggagctgggcctctgcgcccggcccganctccgtctataaanagagcagccagttgcagggctcnantctgctttccaactgcctgactgcttgttcgtctcactggtgtgagctccagcatcccctttgctcgaaatggaccccaactgctcttgcgccactggntggctcctgcacgtgcgccggctcctgcaagtgcaaagagtgcaaatgcacctcctgcaagaagagctgctgttcctgctnccccgtggnctgtgccaagtgtgcccagggctgcgtctgcaaaggggcatcggagaagtgcagctgctgtgcctgatgtgggaacagctcttctcccagatgtaaatagaacaacctgcacaacctggnatttttttaaaaatacaacactgagccatttgctgcatttc

SEQ ID NO: 311 - DNA, 468 bp, human
tactgatgcttctgatactggatgtttagcttcttactgcaaaaacataagtaaaacagtcaactttaccatttccgtattctccatagattgaagaaatttataccacatatcgcatatgaccatctttccatcaaatcaatgtagagataatgtaaactgaaaaaaaatctgcaagataatgtaactgaatgttttaaaaacagaacttgtcactttatataaaagaatagtatgctctatttcctgaatggatgtggaaatgaaagctagcgcacctgcactttgaattcttgcttcttttttattactgttatgattttgctttttacagatgttggacgattttttcttctgattgttgaattcataatcatggtctcatttcctttgcttctttggaatatttctttcaacacattcctttattttattatacattgtgtcctttttttagctattgctgct

SEQ ID NO: 312 - DNA, 147 bp, human
gacaggagggtgtccacatatgttaacatcagttggatctcctatagaagtttctgctgctctctttccttctccctgagctggtaactgcaatgccaacttcctgggcctttctgactagtatcacacttctaataaaatccacaa

SEQ ID NO: 313 - DNA, 560 bp, human
  misc_feature (175)..(177): n is a, c, g, or t
  misc_feature (179)..(181): n is a, c, g, or t
  misc_feature (190)..(190): n is a, c, g, or t
  misc_feature (422)..(422): n is a, c, g, or t
  misc_feature (426)..(426): n is a, c, g, or t
  misc_feature (429)..(430): n is a, c, g, or t
gctccacatgagccatgcatgcttagcaatccaagtgcagagctctttgctccaggagtgaggagactgggaggtgaaatggggaaatggaagggtttggaggcagagctgaaaacagggttggaaggatttcctgaattagaagacaaacgttagcatacccagtaaggaaaannngnnnaggggccanggggaacccgtgaggatcactctcaaatgagattaaaaacaaggaagcagagaatggtcagagaatgggattcagattgggaacttgtggggatgagagtgaccaggttgaactgggaagtggaaaaaggagtttgagtcactggcacctagaagcctgcccacgattcctaggaaggctggcagacaccctggaaccctggggagctactggcaaactctcctggattggncctnatnnttttggtgggaaaggctgccctggggatcaactttccttctgtgtgtggctcaggagttcttctgcagagatggcgctatctttcctcctcctgtgatgtcctgctcccaaccatttgtactcttcatta

SEQ ID NO: 314 - DNA, 530 bp, human
aagtgtgcataatttcatttaacgttaaagaaatagatccaattcctttcttgcaaccaaaaataaataaaatacgttgcctcaatataaggtttgggctattctgtgtttctatagaagcaatctgtttttggtaaaatgtacttttaaggatccagtcatctgaagtattttatgtagagttagagatttcacaatattgactatacatatatttaaaatataaattatccagctgatgtttgaatttgtcttactttcctggccacctcgttgtcctattttataagctggggagttaactagcttaacaaaagatgcttagcttttgtaaaagaacaagtgtttcattttacaaagacactccaaatgatagttacttgattttctcgagacctttaactatggtgatgaataacaggacttgctttcaagccttaataaatgtaaaatgccttttaatgaagatacagctgagtgttttcctcatgaatctgaaccaattaccaatttgtgttccagtcttgatt

SEQ ID NO: 315 - DNA, 417 bp, human
gaaaccaaggtcaggaggccgttgcagacataaatctcggcgactcggccccgtctcctgagggtcctgctggtgaccggcctggaccttggccctacagccctggaggccgctgctgaccagcactgaccccgacctcagagagtactcgcaggggcgctggctgcactcaagaccctcgagattaacggtgctaaccccgtctgctcctccctcccgcagagactggggcctggactggacatgagagccccttggtgccacagagggctgtgtcttactagaaacaacgcaaacctctccttcctcagaatagtgatgtgttcgacgttttatcaaaggccccctttctatgttcatgttagttttgctccttctgtgtttttttctgaaccatatccatgttgctgacttttc

SEQ ID NO: 316 - DNA, 544 bp, human
cacaggcaggtgactactccatgcgcgtggacctgcgggctggggacgaggctgtgttcgcccagtacgactccttccacgtagactcggctgcggagtactaccgcctccacttggagggctaccacggcaccgcaggggactccatgagctaccacagcggcagtgtcttctctgcccgtgatcgggaccccaacagcttgctcatctcctgcgctgtctcctaccgaggggcctggtggtacaggaactgccactacgccaacctcaacgggctctacgggagcacagtggaccatcagggagtgagctggtaccactggaagggcttcgagttctcggtgcccttcacggaaatgaagctgagaccaagaaactttcgctccccagcggggggaggctgagctgctgcccacctctctcgcaccccagtatgactgccgagcactgaggggtcgccccgagagaagagccagggtccttcaccacccagccgctggaggaagccttctctgccagcgatctcgcagcactgtgtttacag

SEQ ID NO: 317 - DNA, 437 bp, human
ccaatggcgagggtctgccacactggccgctgttcgaccaggaggagcaatacctgcagctgaacctacagcctgcggtgggccgggctctgaaggcccacaggctccagttctggaagaaggcgctgccccaaaagatccaggagctcgaggagcctgaagagagacacacagagctgtagctccctgtgccggggaggagggggtgggttcgctgacaggcgagggtcagcctgctgtgcccacacacacccactaaggagaaagaagttgattccttcattcacttcgccattcattcatacttccgtccatccattcagaaagcatttattaagaatttactcaggcatgatggcccatacttgtaatcccagctattgggaaggatgagatgggaggatggcttgaggccagaggtttgagaccgaccagcc

SEQ ID NO: 318 - DNA, 557 bp, human
  misc_feature (464)..(464): n is a, c, g, or t
gggagagtccatctggaagctggagtatatcctgacccagacctacgacattgaagatttgcagccggaaagtttatatggattagctaaacaatttacaatcctagacagtaagcagtttataaaatactacaattacttctttgtgagttatgacagcagtgtaacatgtgataagacatgtaaggcctttcagatttgtgcaattatgaatcttgataatatttcctatgcagattgcctcaaacagctttatataaagcacaattactagtatttcacagtttttgctaatagaaaatgctgattctgattctgagatcaatttgtgggaattttacataaatctttgttaattactgagtgggcaagtagacttcctgtctttgctttctttttttttttctttttgatgccttaatgtagatatctttatcattctgaattgtattatatatttaaantgctcattaatagaatgatggatgtaaattggatgtaaatattcagtttatataattatatctaatttgtacccttgttgaaattgtcattta

SEQ ID NO: 319 - DNA, 336 bp, human
  misc_feature (150)..(150): n is a, c, g, or t
  misc_feature (154)..(154): n is a, c, g, or t
cttcgcttctctcttggaaagtccagtctctcctcggcttgcaatggaccccaactgctcctgcgccgctggtgtctcctgcacctgcgctggttcctgcaagtgcaaagagtgcaaatgcacctcctgcaagaagagctgctgctcctngctnccccgtgggctgtagcaagtgtgcccagggctgtgtttgcaaaggggcgtcagagaagtgcagctgctgcgactgatgccaggacaacctttctcccagatgtaaacagagagacatgtacaaacctggattttttttttataccaccttgacccatttgctacattccttttcctgtgaaa

SEQ ID NO: 320 - DNA, 208 bp, human
  misc_feature (85)..(85): n is a, c, g, or t
  misc_feature (100)..(100): n is a, c, g, or t
  misc_feature (102)..(102): n is a, c, g, or t
  misc_feature (119)..(123): n is a, c, g, or t
  misc_feature (125)..(126): n is a, c, g, or t
  misc_feature (128)..(128): n is a, c, g, or t
  misc_feature (148)..(148): n is a, c, g, or t
cctggggctgagcaaggcctacgtaggccagaagagcagcttcacagtagactgcagcaaagcaggcaacaacatgctgctggtnggggttcatggcccnangaccccctgcgaggagnnnnngnngnagcacgtgggcagccggctntacagcgtgtcctacctgctcaaggacaagggggagtacacactggtggtcaaatggggg

SEQ ID NO: 321 - DNA, 358 bp, human
  misc_feature (146)..(146): n is a, c, g, or t
  misc_feature (180)..(181): n is a, c, g, or t
gaaactgtatgggtagcttttttgtttgttttttgttttgtttttgtttttgtttttgtttttagttgtaggtcgcagcggggaaattttttgcgactgtacacatagctgcagcattaaaaacttaaaaaaattgttaaaaaaanaaaaaaagggaaaacatttcaaaaaaaaaaaaanngataaacagttacaccttgttttcaatgtgtggctgagtgcctcgattttttcatgtttttggtgtatttctgatttgtagaagtgtccaaacaggttgtgtgctggagttccttcaagacaaaaacaaacccagcttggtcaaggccattacctgtttcccatctgtagttattcg

SEQ ID NO: 322 - DNA, 344 bp, human
gctgctgccgcggggaagcaggaactggccaagtacttcttggcagagctgctgtctgaacccaaccagacggagaatgatgccctggaacctgaagatctgtcccaggctgctgagcaggatgaaatgaggcttgagctgcagagatctgctaactcaaacccggctatggcaccccgagaacgcaaagctggctgcaagaatttcttctggaagactttcacatcctgttagctttcttaactagtattgtccatatcagacctctgatccctcgcccccacaccccatctctcttccctaatcctccaagtcttcagcgagacccttgcattagaaactga

SEQ ID NO: 323 - DNA, 486 bp, human
  misc_feature (103)..(103): n is a, c, g, or t
acaggaaattgctacttaccgccgccttctggaaggagaagacgtaaaaactacagaatatcagttaagcaccctggaagagagagatataaagaaaaccagnaagattaagacagtcgtgcaagaagtagtggatggcaaggtcgtgtcatctgaagtcaaagaggtggaagaaaatatctaaatagctaccagaaggagatgctgctgaggttttgaaagaaatttggctataatcttatctttgctccctgcaagaaatcagccataagaaagcactattaatactctgcagtgattagaaggggtggggtggcgggaatcctatttatcagactctgtaattgaatataaatgttttactcagaggagctgcaaattgcctgcaaaaatgaaatccagtgagcactagaatatttaaaacatcattactgccatctttatcatgaagcacatcaattacaagctgtagaccacctaatatca

SEQ ID NO: 324 - DNA, 299 bp, human
gagaaagccaggcaggtagccagggggactagcccctgtggagactggggggcttgaaattgtccccgtggtctcttactttcctttccccagcccagggtggacttagaaagcaggggctacaagagggaatccccgaaggtgctggaggtgggagcaggagattgagaaggagagaaagtgggtgagatgctggagaagagaggagaggagagaggcagagagcggtctcaggctggtgggaggggcgcccacctccccacgccctcccctcccctgctgcaggggctctggagaga

SEQ ID NO: 325 - DNA, 419 bp, human
atgagccttcatacatggactgagagtcagagcttgaagaaaagcttatttattttccccaacctcccccaggtgcagtgtgacattattttattataacatccacaaagagattatttttaaataatttaaagcataatatttcttaaaaagtatttaattatatttaagttgttgatgttttaactctatctgtcatacatcctagtgaatgtaaaatgcaaaatcctggtgatgtgttttttgtttttgttttcctgtgagctcaactaagttcacggcaaaatgtcattgttctccctcctacctgtctgtagtgttgtggggtcctcccatggatcatcaaggtgaaacactttggtattctttggcaatcagtgctcctgtaagtcaaatgtgtgctttgtactgctgttgtt

SEQ ID NO: 326 - DNA, 445 bp, human
  misc_feature (42)..(42): n is a, c, g, or t
  misc_feature (51)..(51): n is a, c, g, or t
  misc_feature (197)..(197): n is a, c, g, or t
  misc_feature (212)..(212): n is a, c, g, or t
  misc_feature (220)..(220): n is a, c, g, or t
  misc_feature (275)..(275): n is a, c, g, or t
  misc_feature (288)..(288): n is a, c, g, or t
  misc_feature (293)..(293): n is a, c, g, or t
  misc_feature (300)..(300): n is a, c, g, or t
  misc_feature (306)..(308): n is a, c, g, or t
  misc_feature (336)..(336): n is a, c, g, or t
  misc_feature (359)..(359): n is a, c, g, or t
atgcctacaggtatgcgtgattgtgtgtgtgtgcatgggtgnacagccacntgtctacctatgtgtctttctgggaatgtgtaccatctgtgtgcctgcagctgtgtagtgctggacagtgacaaccctttctctccagttctccactccaatgataatagttcacttacacctaaacccaaaggaaaaaccagctntaggtccaattgttntgctctanactgatacctcaaccttggggccagcatctcccactgcctccaaatattagtaantatgactgacgtncccanaagtttntgggtnnnaccacactccccaaccccccactcctanttcctgaagggccctcccaaggntacatccccaccccacagttctccctgagagagatcaacctccctgagatcaaccaaggcagatgtgacagcaagggccacggacc

SEQ ID NO: 327 - DNA, 524 bp, human
  misc_feature (40)..(40): n is a, c, g, or t
  misc_feature (44)..(44): n is a, c, g, or t
  misc_feature (48)..(49): n is a, c, g, or t
  misc_feature (52)..(56): n is a, c, g, or t
  misc_feature (60)..(60): n is a, c, g, or t
  misc_feature (98)..(98): n is a, c, g, or t
gcctctggcaatgccattcaggccaggtcttcctcctatngtgnagannatnnnnntggnggtggaaagcgattctctcattctggcaaccagttggnacggccccatcaccgccctccgggtccgagtcaacacatactacatcgtaggtcttcaggtgcgctatggcaaggtgtggagcgactatgtgggtggtcgcaacggagacctggaggagatctttctgcaccctggggaatcagtgatccaggtttctgggaagtacaagtggtacctgaagaagctggtatttgtgacagacaagggccgctatctgtcttttgggaaagacagtggcacaagtttcaatgccgtccccttgcaccccaacaccgtgctccgcttcatcagtggccggtctggttctctcatcgatgccattggcctgcactgggatgtttaccccactagctgcagcagatgctgagcctcctctccttggcaggggcactgtgatgaggagtaagaactcccttatcactaac

SEQ ID NO: 328 - DNA, 335 bp, human
acaaggcccaggctggggccagggccagaggggaaggccctggattctcactcatgtgagatcttgaatctctttctttgttctgtttgtttagttagtatcatctggtaaaatagttaaaaaacaacaaaaaactctgtatctgtttctagcatgtgctgcattgactctattaatcacatttcaaattcaccctacattcctctcctcttcactagcctctctgaaggtgtcctggccagccctggagaagcactggtgtctgcagcacccctcagttcctgtgcctcagcccacaggccactgtgataatggtctgtttagcacttctgtat

SEQ ID NO: 329 - DNA, 244 bp, human
  misc_feature (74)..(74): n is a, c, g, or t
  misc_feature (93)..(93): n is a, c, g, or t
  misc_feature (164)..(169): n is a, c, g, or t
  misc_feature (171)..(172): n is a, c, g, or t
  misc_feature (176)..(176): n is a, c, g, or t
  misc_feature (180)..(181): n is a, c, g, or t
tcaacttcaagctcctaagccactgcctgctggtgaccctggccgcccacctccccgccgagttcaacccctgnggtgcacgcctccctgganaagttcctggcttctgtgagcaccgtgctgacctccaaataccgttaagctggagcctcggtagccgttcnnnnnncnngctnggcnntccaacgggccctcctcccctccttgcaccggcccttcctggtctttgaataaagtctgagtg

SEQ ID NO: 330 - DNA, 543 bp, human
  misc_feature (324)..(324): n is a, c, g, or t
cttcctcggaggcagcattgttaagggaggccctgtgcaagtgctggaggacgaggaactaaagtcccagccagagcccctagtggtcaagggaaaacgggtggctggaggccctcagatgatccagctcagcctggatgggaagcgcctctacatcaccacgtcgctgtacagtgcctgggacaagcagttttaccctgatctcatcagggaaggctctgtgatgctgcaggttgatgtagacacagtaaaaggagggctgaagttgaaccccaacttcctggtggacttcgggaaggagccccttggcccagcccttgcccnatgagctccgctaccctgggggcgattgtagctctgacatctggatttgaaggctccaccctcatcacccacactccctattttgggccctcacttccttggggacctggcttcattctgctctctcttggcacccgacccttggcagcatgtaccacacagccaagctgagactgtggcaatgtgttgagtcatatacatttactgaccactgttgct

SEQ ID NO: 331 - DNA, 507 bp, human
ccatcggtgcggactccaaggaggtgcactataagaaccgcttctggcatgacacctgcttccgctgtgccaagtgccttcaccccttggccaatgagacctttgtggccaaggacaacaagatcctgtgcaacaagtgcaccactcgggaggactcccccaagtgcaaggggtgcttcaaggccattgtggcaggagatcaaaacgtggagtacaaggggaccgtctggcacaaagactgcttcacctgtagtaactgcaagcaagtcatcgggactggaagcttcttccctaaaggggaggacttctactgcgtgacttgccatgagaccaagtttgccaagcattgcgtgaagtgcaacaagggtttggtaaaggctccagtgtggtggcctatgaaggacaatcctggcacgactactgcttccactgcaaaaaatgctccgtgaatctggccaacaagcgctttgttttccaccaggagcaagtgtattgtcccgactgtgc

SEQ ID NO: 332 - DNA, 492 bp, human
ttgtcaaggggctttgcattcaaactgcttttccagggctatactcagaagaaagataaaagtgtgatctaagaaaaagtgatggttttaggaaagtgaaaatatttttgtttttgtatttgaagaagaatgatgcattttgacaagaaatcatatatgtatggatatattttaataagtatttgagtacagactttgaggtttcatcaatataaataaaagagcagaaaaatatgtcttggttttcatttgcttaccaaaaaaacaacaacaaaaaaagttgtcctttgagaacttcacctgctcctatgtgggtacctgagtcaaaattgtcatttttgttctgtgaaaaataaatttccttcttgtaccatttctgtttagttttactaaaatctgtaaatactgtatttttctgtttattccaaatttgatgaaactgacaatccaatttgaaagtttgtgtcgacgtctgtctagcttaaatgaatg

SEQ ID NO: 333 - DNA, 251 bp, human
tcataagtgacttctacccgggagccgtgacagtggcctggaaggcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttcataggttctcaaccctcac

SEQ ID NO: 334 - DNA, 519 bp, human
  misc_feature (118)..(118): n is a, c, g, or t
  misc_feature (125)..(125): n is a, c, g, or t
  misc_feature (133)..(133): n is a, c, g, or t
  misc_feature (136)..(136): n is a, c, g, or t
gagtatacatcggtgcaggcttcctggatgacagttgggtgatatgtgtcatgtggcctaaaagcctccatgtcatttgacctacgaattctatctttgggaatttatcctaagaaantacttanggatttanttngtgataagatgttcatcccagcattgcaatggagaaaaatgggaagcaatggtttggttgggaatttattccttttctgctgtaacgaaagtttgcaataggggattgcttaagtaaattattgtatctccatccagatggtggagtaccgcgcagacattaaaagtcatgtaaaagaacatctgactgaaagaaaaatgctccttgaatattaaaaggttgtaaaaatagtgcatgttatgtgatttcaattttgttttttaaaatatgggtgtatgcttgtatacgtagagcagataaaaaagacggaaggcatactaaaaaatgttgagtggttatctttgtatggtggaacaaagtcactgtaattttcatctttggtt

SEQ ID NO: 335 - DNA, 492 bp, human
  misc_feature (254)..(254): n is a, c, g, or t
  misc_feature (315)..(315): n is a, c, g, or t
  misc_feature (388)..(388): n is a, c, g, or t
tggggctgagcaaggcctacgtaggccagaagagcagcttcacagtagactgcagcaaagcaggcaacaacatgctgctggtgggggttcatggcccaaggaccccctgcgaggagatcctggtgaagcacgtgggcagccggctctacagcgtgtcctacctgctcaaggacaagggggagtacacactggtggtcaaatggggggacgagcacatcccaggcagcccctaccgcgttgtggtgccctgagtntggggcccgtgccagccggcagcccccaagcctgccccgctacccaagcagccccgccctnttcccctcaaccccggcccaggccgccctggccgcccgcctgtcactgcagccgcccctgccctgtgccgtgntgcgctcacctgcctccccagccagccgctgacctctcggctttcacttgggcagagggagccatttggtggcgctgcttgtcttctttggttctgggaggggt

SEQ ID NO: 336 - DNA, 386 bp, human
  misc_feature (30)..(30): n is a, c, g, or t
  misc_feature (117)..(117): n is a, c, g, or t
  misc_feature (123)..(124): n is a, c, g, or t
  misc_feature (221)..(221): n is a, c, g, or t
  misc_feature (331)..(331): n is a, c, g, or t
  misc_feature (335)..(335): n is a, c, g, or t
  misc_feature (338)..(338): n is a, c, g, or t
  misc_feature (344)..(344): n is a, c, g, or t
  misc_feature (354)..(354): n is a, c, g, or t
  misc_feature (359)..(359): n is a, c, g, or t
aatgctctgggtctctggatccagtggggnatattgtgatgactcagtctccactctccctgcccgtcacccctggagagccggcctccatctcctgcaggtctagtcagagcctcntgcatnntaatggatacaactatttggattggtacctgcagaagccagggcagtctccacagctcctgatctatttgggttctaatcgggcctccggggtcccngacaggttcagtggcagtggatcaggcacagattttacactgaaaatcagcagagtggaggctgaggatgttggggtttattactgcatgcaagctctacaaactcctcngacnttnggccangggaccaagntgganatcaaacgaactgtggctgcaccatct

SEQ ID NO: 337 - DNA, 295 bp, human
  misc_feature (136)..(136): n is a, c, g, or t
  misc_feature (221)..(221): n is a, c, g, or t
gcaagatggtgttgcagacccaggtcttcatttctctgttgctctggatctctggtgcctacggggacatcgtgatgacccagtctccagactccctggctgtgtctctgggcgagagggccaccatcaactgcangtccagccagagtgttttatacagctccaacaataagaactacttagcttggtaccagcagaaaccaggacagcctcctaagctnctcatttactgggcatctacccgggaatccggggtccctgaccgattcagtggcagcgggtctgggacagattt

SEQ ID NO: 338 - DNA, 470 bp, human
  misc_feature (92)..(92): n is a, c, g, or t
  misc_feature (126)..(126): n is a, c, g, or t
  misc_feature (148)..(149): n is a, c, g, or t
  misc_feature (166)..(167): n is a, c, g, or t
  misc_feature (170)..(170): n is a, c, g, or t
  misc_feature (182)..(182): n is a, c, g, or t
  misc_feature (239)..(239): n is a, c, g, or t
  misc_feature (269)..(269): n is a, c, g, or t
  misc_feature (289)..(289): n is a, c, g, or t
  misc_feature (291)..(293): n is a, c, g, or t
  misc_feature (317)..(318): n is a, c, g, or t
  misc_feature (320)..(320): n is a, c, g, or t
  misc_feature (327)..(327): n is a, c, g, or t
  misc_feature (329)..(330): n is a, c, g, or t
gaggtgcagctgttggagtctgggggaggcttggtacagcctggggggtccctgagactctcctgtgcagcctctggattcacctttagcanctatgccatgagctgggtccgccaggctccaggnaaggggctggagtgggtctcanntattagtggtagtggtnntancacatactacgnagactccgtgaagggccggttcaccatctccagagacaattccaagaacacgctgtntctgcaaatgaacagcctgagagccgaggncacggccgtatattactgtncnnnagaaaatggtgtcagcagttgganntnccaaganannaccccaacctatgttgactactggggccaggaaccctggtcaccgtctcctcagggagtgcatccgccccaacccttttccccctcgtctcctgtgagaattccccgtcggatacgaagcagcgtggccgttggctgctc

SEQ ID NO: 339 - DNA, 473 bp, human
cgtcagtcatcctttattgcagtcgggatccttggggttgttctgttggccattttcgtcgcattattcttcttgactaaaaagcgaagacagagacagcggcttgcagtttcctcaagaggagagaacttagtccaccaaattcaataccgggagatgaattcttgcctgaatgcagatgatctggacctaatgaattcctcagaaaattcccatgagtcagctgatttcagtgctgctgaactaatttctgtgtctaaatttcttcctatttctggaatggaaaaggaggccattctgagccacactgaaaaggaaaatgggaatttataacccagtgagttcagcctttaagataccttgatgaagacctggactattgaatggagcagaaattcacctctctcactgactattacagttgcatttttatggagttcttcttctcctaggattcctaagactgctgctga

SEQ ID NO: 340 - DNA, 123 bp, human
ggggaatggagcaaaacagtctttgaatatcgaacacgcaaggctgtgagactacctattgtagatattgcaccctatgacattggtggtcctgatcaagaatttggtgtggacgttggccct

SEQ ID NO: 341 - DNA, 290 bp, human
cttgctctgttgcagattggcagatgccgcctccctatgtggtgctggacttgccgcaggagaccctggaggaggagacccccggcgccaacctgtggcccaccaccatcaccttcctcaccctcttcctgctgagcctgttctatagcacagcactgaccgtgaccagcgtccggggcccatctggcaacagggagggcccccagtactgagcgggagccggcaaggcacagggaggaagtgtggaggaacctcttggagaagccagctatgcttgccagaactcagcc

SEQ ID NO: 342 - DNA, 276 bp, human
  misc_feature (32)..(35): n is a, c, g, or t
  misc_feature (203)..(203): n is a, c, g, or t
  misc_feature (240)..(240): n is a, c, g, or t
gcataaattaatcagccccagagtgctttaannnnttctcttaaataccggtgtctttgatcaggatgacatgtgccatttttcagaggacgtgcagacaggctggcattctagattacttttcttactctgaaacatggcctgtttgggagtgcgggattcaaaggtggtcccaccgctgcccctactgcaaatggcagttntaatcttatcttttggcttctgcagatggttgcaatngatccttaaccaataatggtcagtcctcatctctgt

SEQ ID NO: 343 - DNA, 314 bp, human
  misc_feature (150)..(150): n is a, c, g, or t
  misc_feature (161)..(161): n is a, c, g, or t
  misc_feature (185)..(185): n is a, c, g, or t
ctactctggctccgaggtgccagatgtgacatccagatgacccagtctccatcctccctgtctgcatctgtaggagacagagtcaccatcacttgccgggcaagtcagagcattagcagctatttaaattggtatcagcagaaaccaggnaaagcccctangctcctgatctatgctgcatccantttgcaaagtggggtcccatcaaggttcagtggcagtggatctgggacagatttcactctcaccatcagcagtctgcaacctgaagattttgcaacttactactgtcaacagagttacagtacccctca

SEQ ID NO: 344 - DNA, 448 bp, human
  misc_feature (28)..(28): n is a, c, g, or t
  misc_feature (40)..(40): n is a, c, g, or t
  misc_feature (49)..(50): n is a, c, g, or t
  misc_feature (86)..(87): n is a, c, g, or t
  misc_feature (99)..(99): n is a, c, g, or t
  misc_feature (121)..(121): n is a, c, g, or t
  misc_feature (139)..(139): n is a, c, g, or t
  misc_feature (168)..(168): n is a, c, g, or t
  misc_feature (180)..(180): n is a, c, g, or t
  misc_feature (212)..(212): n is a, c, g, or t
  misc_feature (226)..(226): n is a, c, g, or t
  misc_feature (232)..(232): n is a, c, g, or t
  misc_feature (236)..(236): n is a, c, g, or t
  misc_feature (248)..(248): n is a, c, g, or t
  misc_feature (255)..(255): n is a, c, g, or t
  misc_feature (266)..(266): n is a, c, g, or t
  misc_feature (314)..(314): n is a, c, g, or t
  misc_feature (369)..(369): n is a, c, g, or t
  misc_feature (371)..(371): n is a, c, g, or t
  misc_feature (398)..(398): n is a, c, g, or t
gccgagtactggaacagccagaaggacntcctggaagacnagcgggccnnggtggacacctactgcagacacaactacggggttgnngagagcttcacngtgcagcggcgagtccatcctnaggtgactgtgtatcctncaaagacccagcccctgcagcaccacaanctcctggtctgntctgtgagtggtttctatccaggcagcattgnaagtcaggtggttncggaanggcnaggaagagaagnctggggntggtgtccacnggcctgatccagaatggagactggaccttccagaccctggtgatgctngaaacagttcctcggagtggagaggtttacacctgccaagtggagcacccaagcntnacgagccctctcacagtggaatggagngcacggtctgaatctgcacagagcaagatgctgagtggagtcgggggctt

SEQ ID NO: 345 - DNA, 550 bp, human
  misc_feature (30)..(30): n is a, c, g, or t
  misc_feature (42)..(42): n is a, c, g, or t
  misc_feature (49)..(49): n is a, c, g, or t
  misc_feature (58)..(58): n is a, c, g, or t
  misc_feature (116)..(116): n is a, c, g, or t
  misc_feature (119)..(119): n is a, c, g, or t
  misc_feature (175)..(176): n is a, c, g, or t
  misc_feature (178)..(178): n is a, c, g, or t
  misc_feature (180)..(181): n is a, c, g, or t
  misc_feature (183)..(183): n is a, c, g, or t
  misc_feature (188)..(188): n is a, c, g, or t
  misc_feature (225)..(225): n is a, c, g, or t
  misc_feature (246)..(246): n is a, c, g, or t
  misc_feature (248)..(248): n is a, c, g, or t
  misc_feature (258)..(258): n is a, c, g, or t
  misc_feature (268)..(268): n is a, c, g, or t
  misc_feature (274)..(274): n is a, c, g, or t
  misc_feature (285)..(287): n is a, c, g, or t
  misc_feature (289)..(289): n is a, c, g, or t
  misc_feature (299)..(299): n is a, c, g, or t
  misc_feature (336)..(336): n is a, c, g, or t
  misc_feature (364)..(364): n is a, c, g, or t
aagatcctggagtttgtggggcgctccctnccagaggagacngtggacntcatggttnagcacacgtcgttcaaggagatgaagaagaaccctatgaccaactacaccaccgtccnccnggagttcatggaccacagcatctcccccttcatgaggaaaggcatggctggggacnngngnngnccacnttcaccgtggcgcagaatgagcgcttcgatgcggacntatgcggagaagatggcaggncngcagcctcangcttccgctntgagcngtgagaggggnnncntggagtcacngcagagggagtgtgcgaatcaaacctgaccaagcggntcaagaataaaatatgaattgagggccngggacggtaggtcatgtctgtaatcccagcaatttggaggctgaggtgggaggatcatttgagcccaggagttcgagaccaacctgggcaacatagtgagattctgttaaaaaaataaaataaaataaaaccaatttttaaaaagagaataaaatatgattgtgggccaggcagagtggctcatgc

SEQ ID NO: 346 - DNA, 496 bp, human
  misc_feature (150)..(151): n is a, c, g, or t
  misc_feature (345)..(345): n is a, c, g, or t
  misc_feature (347)..(348): n is a, c, g, or t
  misc_feature (366)..(366): n is a, c, g, or t
  misc_feature (405)..(405): n is a, c, g, or t
  misc_feature (428)..(428): n is a, c, g, or t
ccggtcagcaggatcatcgtgcacccacagttctacatcatccagactggagcggatatcgccctgctggagctggaggagcccgtgaacatctccagccgcgtccacacggtcatgctgccccctgcctcggagaccttccccccgggnntgccgtgctgggtcactggctggggcgatgtggacaatgatgagcccctcccaccgccatttcccctgaagcaggtgaaggtccccataatggaaaaccacatttgtgacgcaaaataccaccttggcgcctacacgggagacgacgtccgcatcatccgtgacgacatgctgtgtgccgggaacacccggagngnntcatgccagggcgactcnggagggcccctggtgtgcaaggtgaatggcacctggctncaggcgggcgtggtcagctgggncgagggctgtgcccagcccaaccggcctggcatctacacccgtgtcacctactacttggactggatcc

SEQ ID NO: 347 - DNA, 519 bp, human
  misc_feature (326)..(326): n is a, c, g, or t
  misc_feature (374)..(376): n is a, c, g, or t
  misc_feature (398)..(401): n is a, c, g, or t
gaaccacctcaatgcaaagattctacgggaaaatgtgggccccctccacctattgacaatggggacattacttcattcccgttgtcagtatatgctccagcttcatcagttgagtaccaatgccagaacttgtatcaacttgagggtaacaagcgaataacatgtagaaatggacaatggtcagaaccaccaaaatgcttacatccgtgtgtaatatcccgagaaattatggaaaattataacatagcattaaggtggacagccaaacagaagctttatttgagaacaggtgaatcagctgaatttgtgtgtaaacggggatatcngtctttcatcacgttctcacacattgcgaacaacatgttgggatgggnnnctggagtatccaacttgtgcannnngatagaatcaatcataaaatgcacacctttattcagaactttagtattaaatcagttcttaatttcatttttaagtattgttttactcctttttattcatacgtaaaattttggatta

SEQ ID NO: 348 - DNA, 516 bp, human
  misc_feature (161)..(162): n is a, c, g, or t
  misc_feature (164)..(167): n is a, c, g, or t
  misc_feature (170)..(170): n is a, c, g, or t
  misc_feature (174)..(176): n is a, c, g, or t
  misc_feature (179)..(179): n is a, c, g, or t
  misc_feature (256)..(256): n is a, c, g, or t
  misc_feature (364)..(370): n is a, c, g, or t
  misc_feature (372)..(376): n is a, c, g, or t
  misc_feature (465)..(465): n is a, c, g, or t
tcttccaatttattatctgcaccaattattttgggcacttctcctgtgtatgtaaactttggagtttaggtctacgtcatttatataagagcctgtagacctgagtggatggacactgcctcttagaactagaacttagaactttatcttgaaaatgtacnncnnnngcngaannnccncacagagtatgtgtcaggtaggaaccaaatttatgattgttatattcaaactggttttctgttccttgaaaatggtnttttttctgatcaaagcctatttaggagttattatgttcttgagtttattccatgtgtgtgaatattttgaatgaatccactaataagatatgcatgtatgtctggannnnnnncnnnnnagaataaatatttaagtagactttaacaagtaaatggttgtaggcatcattaagtcttaaatagccttttcaaagatattagtgttctngttttaattattcagataattcctttaattctccactcaaatctgacttta

SEQ ID NO: 349 - DNA, 507 bp, human
gattcctgtgggtccagctttggaactgggaaacctttcttcggatccgcactcattccactgatgccagctgcccctgaaggatgccagtactgtggtgtgtgagtctcagcagccgcccacacgctcctaactctgctgcatggcagatgcctaggtggaaatagcaaaaacaaggcccgggctggggccagggccagaggggaaggccctggattctcactcatgtgagatcttgaatctctttctttgttctgtttgtttagttagtatcatctggtaaaatagttaaaaaacaacaaaaaactctgtatctgtttctagcatgtgctgcattgactctattaatcacatttcaaattcaccctacattcctctcctcttcactagcctctctgaaggtgtcctggccagccctggagaagcactggtgtctgcagcacccctcagttcctgtgcctcagcccacaggccactgtgataatggtctgtttagcacttctgtat

SEQ ID NO: 350 - DNA, 275 bp, human
  misc_feature (129)..(129): n is a, c, g, or t
  misc_feature (187)..(187): n is a, c, g, or t
  misc_feature (191)..(191): n is a, c, g, or t
ctctggctcccaggtgccagatgtgccatccagttgacccagtctccatcctccctgtctgcatctgtaggagacagagtcaccatcacttgccgggcaagtcagggcattagcagtgctttagcctgntatcagcagaaaccagggaaagctcctaagctcctgatctatgatgcctccagtttgnaaantggggtcccatcaaggttcagcggcagtggatctgggacagatttcactctcaccatcagcagcctgcagcctgaagattttgc

SEQ ID NO: 351 - DNA, 339 bp, human
ccaactgcctgactgcttgttggcctcaccggtgggagctccagcatctcctttgctcgaaatggaccccaactgctcctgtgccactggtggctcctgcatgtgcgccggctcctgcaagtgcaaagagtgcaaatgcacctcctgcaagaagagctgctgctcctgctgccccgtgggctgtgccaagtgtgcccagggctgcatctgcaaaggggcgttggagaagtgcaactgctatgcctgacgtggggacagctctgctcccagatgtaaatagagtaacctgcacaaacctggattttttaaaaaatacaacactgagccatttgcttcatt

SEQ ID NO: 352 - DNA, 259 bp, human
tcctccctgtctgcatctgtaggagacagagtcaccatcacttgccgggtgagtcagggcattagcagttatttaaattggtatcggcagaaaccagggaaagttcctaagctcctgatctatagtgcatccaatttgcaatctggagtcccatctcggttcagtggcagtggatctgggacagatttcactctcactatcagcagcctgcagcctgaagatgttgcaacttattacggtcaacggacttacaatgccc

SEQ ID NO: 353 - DNA, 539 bp, human
tggaggaagttctccagcttcagctcaactcacagcttctccaagcatcaccctgggagtttcctgagggttttctcataaatgagggctgcacattgcctgttctgcttcgaagtattcaataccgctcagtattttaaatgaagtgattctaagatttggtttgggatcaataggaaagcatatgcagccaaccaagatgcaaatgttttgaaatgatatgaccaaaattttaagtaggaaagtcacccaaacacttctgctttcacttaagtgtctggcccgcaatactgtaggaacaagcatgatcttgttactgtgatattttaaatatccacagtactcactttttccaaatgatcctagtaattgcctagaaatatctttctcttacctgttatttatcaatttttcccagtatttttatacggaaaaaattgtattgaaaacacttagtatgcagttgataagaggaatttggtataattatggtgggtgattattttttatactgtatgtgccaaagctttactactgtg

SEQ ID NO: 354 - DNA, 358 bp, human
ccgccatttcctctgaagcaggtgaaggtccccataatggaaaaccacatttgtgacgcaaaataccaccttggcgcctacacgggagacgacgtccgcatcgtccgtgacgacatgctgtgtgccgggaacacccggagggactcatgccagggcgactccggagggcccctggtgtgcaaggtgaatggcacctggctgcaggcgggcgtggtcagctggggcgagggctgtgcccagcccaaccggcctggcatctacacccgtgtcacctactacttggactggatccaccactatgtccccaaaaagccgtgagtcaggcctgggttggccacctgggtcactggaggaccaa

SEQ ID NO: 355 - DNA, 273 bp, human
agtcgggcccaggactggtgaagccttcggagaccctgtccctcacctgcactgtctctggtggctccatcagtagttactactggagctggatccggcagcccgccgggaagggactggagtggattgggcgtatctataccagtgggagcaccaactacaacccctccctcaagagtcgagtcaccatgtcagtagacacgtccaagaaccagttctccctgaagctgagctctgtgaccgccgcggacacggccgtgtattactgtgcga

SEQ ID NO: 356 - DNA, 162 bp, human
  misc_feature (114)..(114): n is a, c, g, or t
  misc_feature (119)..(119): n is a, c, g, or t
agtgggtctcagctattagtgggagtgcaggtaccacatactacgcagactccgtgaagggccggttcaccacctccagagacaattccaagaacacgctgtatctgcaaatgnacagnctgagagccgaggacacggccgtatattactgtgcgaaatccc

SEQ ID NO: 357 - DNA, 288 bp, human
tcctccctgtctgcatctgtaggagacagagtcaccatcacttgccaggcgagtcaggacattagtaattttttaacttggtatcagaagaagccagggaaagcccctaaagtcctgatctacgatgcatccaatttggaaacaggggtcccatcaaggttcagtggaagtggttctgggacagattttactttcaccatcgccagcctgcagcctgacgattttgcaacatattactgtcaacaatataatgatcaccccctcactttcggccctgggaccaaagtg

SEQ ID NO: 358 - DNA, 356 bp, human
atgaaactgattacaacaggctgtaagaatcaaagtcaactgacatctatgctacatattattatatagtttgtactgagctattgaagtcccattaacttaaagtatatgttttcaaattgccattgctactattgcttgtcggtgtattttattttattgtttttgactttggaagagatgaactgtgtatttaacttaagctattgctcttaaaaccagggatcagaatatatttgtaagttaaatcattggtgctaataataaatgtggattttgtattaaaatatatagaagcaatttctgtttacatgtccttgctacttttaaaaacttgcatttattcctcagatttt

SEQ ID NO: 359 - DNA, 247 bp, human
catctcctgcactggaaccagcagtgacgttggtgcttataactatgtctcctggtaccaacagcacccaggcaaagcccccaaactcatgatttatgaggtcagtaatcggccctcaggggtttctaatcgcttctctggctccaagtctggcaacacggcctccctgaccatctctgggctccaggctgacgacgagggtgattactactgcatctcatatacaagtagcaaccctctcgtggtt

SEQ ID NO: 360 - DNA, 548 bp, human
tcaagtgggaagagcgctgttcaaggaccacctgagcgtgacctctgtggctgctacagcgtgttccagtgtcctgccgggctgtgccgagccatggaaccatggggagaccttcacttgcactgctgcccaccccgagttgaagaccccactaaccgccaacatcacaaaatccggaaacacattccggcccgaggtccacctgctgccgccgccgtcggaggagctggccctgaacgagctggtgacgctgacgtgcctggcacgtggcttcagcccaaggatgtgctggttcgctggctgcaggggtcacaggagctgccccgcgagaagtacctgacttgggcatcccggcaggagcccagccagggcaccaccaccttcgctgtgaccagcatactgcgcgtggcagccgaggactggaagaagggggacaccttctcctgcatggtgggccacgaggccctgccgctggccttcacacagaagaccatcgaccgcttggcgggtaaacccacccatgtcaatgtgtctgttgtcatggcgga

SEQ ID NO: 361 - DNA, 257 bp, human
gctgtcaggattcttcaaggagaatgaatactgggaatcaagacaggactataccttatccataggcgcaggtgcacagggggaggccataaagatcaaacatgcatggatgggtcctcacgcagacacacccacagaaggacactagcctggcgcgcgtgcacacacacacacacacacacgagttcataatgtggtgatggccctaagttaagcaaaatgcttctgcacacaaaactctctggtttacttcaaat

SEQ ID NO: 362 - DNA, 277 bp, human
gcacctgagcatgaaactcgacgcgttcttcggcatcttctttggggccctgggcggcctcttgctgctgggggtcgggacgttcgtggtcctgcgcttctggggttgctccggggccaggttctcctatttcctgaactcagctgaggccttgccttgaaggggcagctgtggcctaggctacctcaagactcacctcatccttaccgcacatttaaggcgccattgcttttgggagactggaaaagggaaggtgactgaaggctgtcaggattct

SEQ ID NO: 363 - DNA, 140 bp, human
  misc_feature (33)..(33): n is a, c, g, or t
  misc_feature (44)..(44): n is a, c, g, or t
  misc_feature (64)..(65): n is a, c, g, or t
  misc_feature (99)..(100): n is a, c, g, or t
catctcctgcactggaaccagcagtgacgttgntggttataacnatgtctcctggtaccaacannacccaggcaaagcccccaaactcatgatttatgnngtcagtaatcggccctcaggggtttctaatcgcttctctg

SEQ ID NO: 364 - DNA, 113 bp, human
gcgctggttcctgcaagtgcaaagagtgcaaatgcacctcctgcaagaagagctgctgctcctgctgccccgtgggctgtagcaagtgtgcccagggctgtgtttgcaaaggg

SEQ ID NO: 365 - DNA, 266 bp, human
aggacagagggtcaccatctcttgctctggaagcagctccaacattgggaagaattatgtatcctggtaccagcaactcccaggaacagcccccaaactcctcatctataaaaataataagcgaccctcagggattcctgaccgattctctggctccaagtctggcacgtcagccaccctgggcatcaccagactccagactggggacgaggccgattattactgcggaacatgggatagcagcctgagtggtggccgggggatat

SEQ ID NO: 366 - DNA, 434 bp, human
tgaggagaagtctgccgttactgccctgtggggcaaggtgaacgtggatgaagttggtggtgaggccctgggcaggctgctggtggtctacccttggacccagaggttctttgagtcctttggggatctgtccactcctgatgctgttatgggcaaccctaaggtgaaggctcatggcaagaaagtgctcggttctttgagtcctttggggatctgtccactcctgatgctgttatgggcaaccctaaggtgaaggctcatggcaagaaagtgctcggtgcctttagtgatggcctggctcacctggacaacctcaagggcacctttgccacactgagtgagctgcactgtgacaagctgcacgtggatcctgagaacttcaggctcctgggcaacgtgctggtctgtgtgctggcccatcactttggcaaaga

SEQ ID NO: 367 - DNA, 273 bp, human
cagtgtccggatctcctggacagtcagtcaccatctcctgcaccggaaccagcagtgatgttggtggttataactatgtctcctggtaccaacagcacccaggcaaagcccccacactcatgatttatgatgtcattaagcggccctcaggggtccctgatcgcttctctggctccaaatctggcaacacggcctccctgaccatctctgggctccaggctgaggatgaggctgattattattgcagctcaaatgcaggcagtttttatgtct

SEQ ID NO: 368 - DNA, 233 bp, human
tcctgcactgggagcagctcctacatcggggcaggttatgatgtacactggtaccggcaacttcccggcacagcccccaaactcctcatctatggtaacaccgatcggccctcaggggtccctgaccgattctctggctccaagtctggcatctcagcctccctggccatcactgggctccaggctgaggatgaggctgattattactgcaagtcctatgacagcagcctgag

SEQ ID NO: 369 - DNA, 256 bp, human
gatgtgacatccaggtgacccagtctccatcttccctgtctgcgtctgtaggagacagagtcaccatcacctgccgggcaagtcagggcattagcaatgggttatcctggtatcagcagaaaccagggcaagcccctacgctcctgatctatgctgcatccagtttgcagtcgggggtcccatctcggttcagtggcagtggatctgggacagatttcactctcaccatcagcagcctgcagcctgaagatgttgc

SEQ ID NO: 370 - DNA, 434 bp, human
ggtccccacagactcagagagaacccaccatggtgctgtctcctgccgacaagaccaacgtcaaggccgcctggggtaaggtcggcgcgcacgctggcgagtatggtgcggaggccctggagaggcacttcgacctgagccacggctctgcccaagttaagggccacggcaagaaggtggccgacgcgctgaccaacgccgtggcgcacgtggacgacatgcccaacgcgctgtccgccctgagcgacctgcacgcgcacaagcttcgggtggacccggtcaacttcaagctaagccactgcctgctggtgaccctggccgcccacctccccgccgagttcacccctgcggtgcacgcttccctggacaagttcctggcttctgtgagcaccgtgctgacctccaaataccgttaagctggagcctcggtagcc

SEQ ID NO: 371 - DNA, 270 bp, human
gctgccaggtgccagatgtgacatccagatgacccagtctccatcctccctgtctgcatctgtaggagacagagtcaccatcacttgccgggcgagtcagggcattagcaataatttaaattggtatcagcagaaaccagggaaaactcctaagctcctgatctatgctgcacccagtctgcaaagtgggattccctctcggttcagtgacagtggatctggggcagattacactctcaccatccgcagcctgcagcctgaagattttgc

SEQ ID NO: 372 - DNA, 319 bp, human
agtcgctccatttatcgcttgagatctccagccttaccgcggctcgaaatggaccccaactgctcctgcaccactggtgtctcctgcgcctgcaccggctcctgcaagtgcaaagagtgcaaatgcacctcctgcaagaagagctgctgctcctgctgccccgtgggctgtgccaagtgtgcccacggctgtgtctgcaaagggacgttggagaactgcagctgctgtgcctgatgtgggaacagctcttctcccagatgttaatagaacaagctgcacaacctggattttttttcaatacgatactgagccatttgct

SEQ ID NO: 373 - DNA, 434 bp, human
cctccagacctccttgaaatacaatattctcccagaaaaggaagagttcccctttgctttaggagtgcagactctgcctcaaacttgtgatgaacccaaagcccacaccagcttccaaatctccctaagtgtcagttacacagggagccgctctgcctccaacatggcgatcgttgatgtgaagatggtctctggcttcattcccctgaagccaacagtgaaaatgcttgaaagatctaaccatgtgagccggacagaagtcagcagcaaccatgtcttgatttaccttgataaggtgtcaaatcagacactgagcttgttcttcacggttctgcaagatgtcccagtaagagatctcaaaccagccatagtgaaagtctatgattactacgagacggatgagtttgcaatcgctgagtacaatgctccttgca

SEQ ID NO: 374 - DNA, 500 bp, human
  misc_feature (77)..(77): n is a, c, g, or t
  misc_feature (181)..(181): n is a, c, g, or t
  misc_feature (207)..(207): n is a, c, g, or t
ctattaccattctaactttcataaaaagttgggatcaagaagcagctgatttcctgccagggcttatattagggggntgattcttaaaggacattaggattggtgctcagaaatggttaatcatgctgtgtgctagccagggccagctggtaccttctttgccatgagcattcaagggacngctaacctttattgacaatctatatngcaaaagtcaggaaagaggttgtgagctgattggattaaagacctggcacttcagtaactcagcacgcttccacttcactcaacttaagagagttcattgacagtgttaggatgtgaaggctgggaaacacttattttgcttcaagagttccacttggctctcccaaataggtacctcaaaaactgttagcaagcggcatttggatgtcttgacaggggctttgcagggatttttagggttttttccacattgtccacattaatggttggcatgattgtgcttgcaggccaag

SEQ ID NO: 375 - DNA, 542 bp, human
gaatatgacgttaccttgcagactaaaaggttgaaggcccgaaactaacttttagctaacaataagggctgtgccccaatggaaactgagttcattttctgagaaaggtttggatgactgaaatatttcctctacagtcaaggactttggcatgcggtggctgaaactgagcttttttgtgtgggctccagttctcactgttctgcaatgctcatggcaagttgaatggtgagctagcttataaattaaagagctctgaactgtattcagaccgactgggtatctagcttactgttttaacatcattgttgaaaccagaccctgtagtccagtggtgctgccctgttgtgcaaactgctcctttttctcgtgtttttgtaaagagcttccatctgggctggacccagttcttgcacatacaagacaccgctgcagtcagctaggacctttccgccatgtattctattctgtagtaaagcatttccatcaacaatgcctaattgtatctgttatttttggtttaacacacactgattcatact

SEQ ID NO: 376 - DNA, 541 bp, human
ggtctacgcctattacaacctggaggaaagctgtacccggttctaccatccggaaaaggaggatggaaagctgaacaagctctgccgtgatgaactgtgccgctgtgctgaggagaattgcttcatacaaaagtcggatgacaaggtcaccctggaagaacggctggacaaggcctgtgagccaggagtggactatgtgtacaagacccgactggtcaaggttcagctgtccaatgactttgacgagtacatcatggccattgagcagaccatcaagtcaggctcggatgaggtgcaggttggacagcagcgcacgttcatcagccccatcaagtgcagagaagccctgaagctggaggagaagaaacactacctcatgtggggtctctcctccgatttctggggagagaagcccaacctcagctacatcatcgggaaggacacttgggtggagcactggcctgaggaggacgaatgccaagacgaagagaaccagaaacaatgccaggacctcggcgccttcaccgagagcatggttgtc

SEQ ID NO: 377 - DNA, 485 bp, human
cccctgcatatcttctcagcaataactccatgggctctgggaccctaccccttccaaccttccctgcttctgagacttcaatctacagcccagctcatccagatgcagactacagtccctgcaattgggtctctggcaggcaatagttgaaggactcctgttccgttggggccagcacaccgggatggatggagggagagcagaggcctttgcttctctgcctacgtccccttagatgggcagcagaggcaactcccgcatcctttgctctgcctgtcggtggtcagagcggtgagcgaggtgggttggagactcagcaggctccgtgcagcccttgggaacagtgagaggttgaaggtcataacgagagtgggaactcaacccagatcccgcccctcctgtcctctgtgttcccgcggaaaccaaccaaaccgtgcgctgtgacccattgctgttctctgtatcgtgatctatcctcaacaaca

SEQ ID NO: 378 - DNA, 512 bp, human
ctcttttactactgacctttcacagagaaaaaatatttcccttgaaaaaaactgggcttgtcattttttcccttgtagctttaagcagagacataagtgccttgcattacacatagtaaactttctttaaaaaaaaaaaaaaaagattttggagactaccagggtaagattccaacttgtccaaaagctttctggccttacatattttattataaaaattctcaagtctggtaatcttctatgtcagagctagtgatttcaaaaggtttcacaattccccaagacaaaagtgattttcgttcattataataaggttaagtgatatgtgattcataacaattttgatgtgaagaagggaaggacatcattgacttaataatagtatcagtcggtgcaacagttggcaacatgtgccttcacactttaccataaagagacgggtttgagggtttgccttctaaagtctgcaacttcaagaaaaaaaatcgacactgtggattgactttcccggt

SEQ ID NO: 379 - DNA, 520 bp, human
aactttgtatagcccatgtacctaccttgtatagaaaaataattttaaaaatttgaatggaagggggtaaaggaagtcatgaagtttttttgcatttttatttaaatgaaggaattccaaataactcacctacagatttttagcacaaaaatagccattgtaaagtgttaaaatttacgataagtattctattggggaggaaaggtaactctgatctcagttacagtttttttttcctttttaatttcattattttgggtttttggtttttgcagtcctatttatctgcagtcgtattaagtcctattgctagaataggttactacaaaaaaggttatattctgaaagaaaaataactgacattatatataaccaattaatttaaagtattgccatttaaattacacactgagagcatgtcctatgcagacatagatttttctgttcatttatttttcttcattgcagtggattgatttgataaatagatgtgttgaattactacatttgctgtacatat

SEQ ID NO: 380 - DNA, 448 bp, human
ggagctagccttgttttttgcatctttctcactcccatacatttatattatatccccactaaatttcttgttcctcattcttcaaatgtgggccagttgtggctcaaatcctctatatttttagccaatggcaatcaaattctttcagctcctttgtttcatacggaactccagatcctgagtaatccttttagagcccgaagagtcaaaaccctcaatgttccctcctgctctcctgccccatgtcaacaaatttcaggctaaggatgccccagacccagggctctaaccttgtatgcgggcaggcccagggagcaggcagcagtgttcttcccctcagagtgacttggggagggagaaataggaggagacgtccagctctgtcctctcttcctcactcctcccttcagtgtcctgaggaacaggactttctccacattgttttgta

SEQ ID NO: 381 - DNA, 547 bp, human
  misc_feature (159)..(159): n is a, c, g, or t
  misc_feature (240)..(240): n is a, c, g, or t
  misc_feature (299)..(299): n is a, c, g, or t
  misc_feature (312)..(312): n is a, c, g, or t
gcgggtcttgctggctaaaatgcccaggtaaagggttggttggacacagcgcttagtgcacgctgtcatcatggacatcataatcagttgtgaaaaacacgcgaacctatgacacttcttattccacactgaatgtgaaattgcatgttcagatgtttnactacgaggcctggctcacaggaagtgttcagtaaaagtatgcactgttagattactgataacgcggatagatttttgttnaccataaattgttccagatttatattaatggaaggaagtgtgcatttattagctattantcaactttacaangcaaacatcttatttctcatctttaaacatgtcgaccagtttaattgaaaagtattctgagactgcaaaatggggtgttaaaaaatactgcagttacggagctgtgtaaaccagtttctcattgcataagatacagatgtaaattgcatggagaggttgatatgcacctgtacagtaattcactcccccatttcacttctttgtcagagaatagttcttgttcatactgagtgtt

SEQ ID NO: 382 - DNA, 425 bp, human
gcatccgaggggagccgccggatgtggaagaagactcggctttcctgcagccatttagtgccgccccatgctaggttatttgacattgtgcagtgtagagttgccttaaagtgcgtgatctgccagtgctttcttcaagtcacccttgccccgattcctcatgtttgcgctccccagggttgctcaagtggaaattttgtcagctgtttagccttttcgtacttggcgtgatgtcaacttcacttctaatctgcaaaagcagaagctgtttcctagtttacctcgcgtgtgtttacctatatggagtagctcgcagagatcacagaaatgcttgcagcctaaggcagggttttcagaccgtgggtcccagcccatttagtaaaatgggaaatcaattagcaagtggtcaccagcattacacagca

SEQ ID NO: 383 - DNA, 551 bp, human
tatacagtttaataagcctcttgcaagttacttgttctctcacctgaggtatttttttcctccccaccttgcccctgttcctcccttcctcttctccctttgcaagaggaaatatttaacatatttgggtccaacttcaataatgtaataattaatacattaaaagcatttaacttcctttctagaaaaatgcacaggctaaggcatagacaaaacaaagagaaatgctgagaaatttgccactggagacaagcaatctgaataaatatttgccaaaagttctttttatgtcatatagtgtcaggatttgaaggagctattttttttaatgttgcaactagcaactcatcttcggaagacacagccaggagaatgaagtagaagtgaaaggtttataaatccatttgtaagcatttatcccatatattttaaattcaagaaaaattgtgtttatctttagaattttgtattcaatactttatgtactatgtgactcatgcttctggataaataaagcaccaaatatgtatctgtaaccacaatcacacata

SEQ ID NO: 384 - DNA, 468 bp, human
ggcttgatgtagactggcttgctttgatttttagtgaagggaatgtacgtaaaacaaaatagggcttggctggtcaaaggagacaagcaggatggatggatggatggatgaatagatagatggtgtttgcatgtaaattgcagagaaaacaaaaccaaagctgattggaaacaattaattgtgggtgtctgagggggaaggtcgcagctttgggcagctttgagaagcggtacaagagctctgtgcctgtgtgtccagccctggagccagccagtgcatttattttaagctcttagaagcaactccttggcccaggaatgcgtgacccctgagatgggtccacgcatctctctacacgtccttctctccgtgggatactggactcgtgcctctgcgcccattctcttctcacgcatatccatgagctttaatttcactttctgatcacggtacgtccataaagccagt

SEQ ID NO: 385 - DNA, 355 bp, human
ggagatgacttaagttggcagcagtaatcttcttttaggagcttgtaccacagtcttgcacataagtgcagatttgccccaagtaaagagaatttcctcaacactaacttcacggggataatcaccacgtaactacccttaaagcatatcactagccaaagaggggaatatctgttcttcttactgtgcctatattaagactagtacaaatgtggtgtgtcttccaactttcattgaaaatgccatatctataccatattttattcgagtcactgatgatgtaatgatatattttttcattattatagtagaatatttttatggcaagagatttgtggtcttgatcatacctatt

SEQ ID NO: 386 - DNA, 551 bp, human
gaagctgacacaccgtatgttgttagagtcttttatctggtcaggggaaacaaaatcttgacccagctgaacatgtcttcctgagtcagtgcctgaatctttattttttaaattgaatgttccttaaaggttaacatttctaaagcaatattaagaaagactttaaatgttattttggaagacttacgatgcatgtatacaaacgaatagcagataatgatgactagttcacacataaagtccttttaaggagaaaatctaaaatgaaaagtggataaacagaacatttataagtgatcagttaatgcctaagagtgaaagtagttctattgacattcctcaagatatttaatatcaactgcattatgtattatgtctgcttaaatcatttaaaaacggcaaagaattatatagactatgaggtaccttgctgtgtaggaggatgaaaggggagttgatagtctcataaaactaatttggcttcaagtttcatgaatctgtaactagaatttaattttcaccccaataatgttctatatagcctttgctaa

SEQ ID NO: 387 - DNA, 433 bp, human
gttcaaaatatttccaagcctgagtattgtctattggtatagatttttagaaatcaataattgattatttatttgcacttattacaatgcctgaaaaagtgcaccacatggatgttaagtagaaattcaagaaagtaagatgtcttcagcaactcagtaaaaccttacgccaccttttggtttgtaagaggttttttatacatttcaaacaggttgcacaaaagttaaaataatggggtcttttataaatccaaagtactgtgaaaacattttacatattttttaaatcttctgactaatgctaaaacgtaatctaattaaatttcatacagttactgcagtaagcattaggaagtgaatatgatatacaaaatagtttataaagactctatagtttctataatttattttactggcaaatgtcatgcaacaa

SEQ ID NO: 388 - DNA, 493 bp, human
ggcagcccacaagtttctcgtggggagatggaggcagagcccagggtaggggacagagctgctggggcctttccttgcctgggaatctgtcccaggaagagcttccccactcccatcccccaaattggaaaaaccgtacattcaagcctgtttggccctgaaattcttaagaatctggttaagaattaactcactaatgtcaaaagtcaaaacctcctaggggttgtcctgggagtcaggttcacgggtacagaagatgaatctcagatgtcactcaacctgagccgtcattctctgtggcagggctgccctgggtttctcttactcaatccctggagtgtaagcatttggattgtgtcacagattacctttttaccttttctttctttttttttctttttttcaatatcagtgcccacaccttactgagtattgagttttagagctttcgcttgatgtgcttgaccaagagacttcttttgtatccttttct

SEQ ID NO: 389 - DNA, 473 bp, human
tcaagtgcgttctttagaccaatgcattgcgtttctttccctgcttttgagatagtaggaagagttcttggtggtgtccccccccttcaattcttcagttgtatagtagttatagggaagatatgggtgtttttctttattattacttttttttttctgcaggtcagtaaaaggatttaagttgcactgacaaaaataccaaaataaaagtgtatttttaagttcccatttgaaattgctggcgctgctggccggatgcatttttgagtttgtattagttgataaattaacagtaataacaagattgtatgaaccgcatggtgcttgcagttttaaatattgtggatatttgtcctgcatcagaaacgagctttggtttttacagattcaactgtgttgaaatcaaacctgccgcaacagaaattgtttttatttcatgtaaaataagggatcaatttcaaaccctgcttatg

SEQ ID NO: 390 - DNA, 495 bp, human
cctcctgcctctggattttaggtgttgatttctggatcacgggataccacttcctgtccacaccccgaccaggggctagaaaatttgtttgagatttttatatcatcttgtcaaattgcttcagttgtaaatgtgaaaaatgggctggggaaaggaggtggtgtccctaattgttttacttgttaacttgttcttgtgcccctgggcacttggcctttgtctgctctcagtgtcttccctttgacatgggaaaggagttgtggccaaaatccccatcttcttgcacctcaacgtctgtggctcagggctggggtggcagagggaggccttcaccttatatctgtgttgttatccagggctccagacttcctcctctgcctgccccactgcaccctctcccccttatctatctccttctcggctccccagcccagtcttggcttcttgtcccctcctggggtcatccctccactctgactctgactatggcagcag

SEQ ID NO: 391 - DNA, 482 bp, human
ggcatgtgtgactgtttcagcgactgcggagtctgtctctgtggcacattttgtttcccgtgccttgggtgtcaagttgcagctgatatgaatgaatgctgtctgtgtggaacaagcgtcgcaatgaggactctctacaggacccgatatggcatccctggatctatttgtgatgactatatggcaactctttgctgtcctcattgtactctttgccaaatcaagagagatatcaacagaaggagagccatgcgtactttctaaaaactgatggtgaaaagctcttaccgaagcaacaaaattcagcagacacctcttcagcttgagttcttcaccatcttttgcaactgaaatatgatggatatgcttaagtacaactgatggcatgaaaaaaatcaaatttttgatttattataaatgaatgttgtccctgaacttagctaaatggtgcaacttagtttctccttgctttcatattatcg

SEQ ID NO: 392 - DNA, 566 bp, human
  misc_feature (124)..(124): n is a, c, g, or t
agagaatgcccttctccttattgtaaccctgtctggatcctatcctcctacctccaaagcttcccacggcctttctagcctggctatgtcctaataatatcccactgggagaaaggagttttgncaaagtgcaaggacctaaaacatctcatcagtatccagtggtaaaaaggcctcctggctgtctgaggctaggtgggttgaaagccaaggagtcactgagaccaaggctttctctactgattccgcagctcagaccctttcttcagctctgaaagagaaacacgtatcccacctgacatgtccttctgagcccggtaagagcaaaagaatggcagaaaagtttagcccctgaaagccatggagattctcataacttgagacctaatctctgtaaagctaaaataaagaaatagaacaaggctgaggatacgacagtacactgtcagcagggactgtaaacacagacagggtcaaagtgttttctctgaacacattgagttggaatcactgtttagaacacacacacttactttttctggtctctaccactgctgatattttct

SEQ ID NO: 393 - DNA, 489 bp, human
attaccttatcatgtcttagagcccgtctttatgtttaaaactaatttcttaaaataaagccttcagtaaatgttcattaccaacttgataaatgctactcataagagctggtttggggctatagcatatgcttttttttttttaattattacctgatttaaaaatctctgtaaaaacgtgtagtgtttcataaaatctgtaactcgcattttaatgatccgctattataagcttttaatagcatgaaaattgttaggctatataacattgccacttcaactctaaggaatatttttgagatatccctttggaagaccttgcttggaagagcctggacactaacaattctacaccaaattgtctcttcaaatacgtatggactggataactctgagaaacacatctagtataactgaataagcagagcatcaaattaaacagacagaaaccgaaagctctatataaatgctcagagttctttatgtatt

SEQ ID NO: 394 - DNA, 506 bp, human
atggggctggggacttgaattggatgcttcaaaaccatcacctgttggccaacaagtttgacccaaaggtagatgataatgctcttcagtgcttagaagaatacctacgttataaggccatctatgggactgaactttgagacacactatgagagcgttgctacctgtggggcaagagcatgtacaaacatgctcagaacttgctgggacagtgtgggtgggagaccagggctttgcaattcgtggcatcctttaggataagagggctgctattagattgtgggtaagtagatcttttgccttgcaaattgctgcctgggtgaatgctgcttgttctctcacccctaaccctagtagttcctccactaactttctcactaagtgagaatgagaactgctgtgatagggagagtgaaggagggatatgtggtagagcacttgatttcagttgaatgcctgctggtagcttttccattctgtggagctgccgttcctaataattccag

SEQ ID NO: 395 - DNA, 315 bp, human
ggaattcttctcttacttcaataaaatgggttttaacataactttaaattcagttaaatatacaatattgaatacctatagttgactttgggatggggactttttcaagtcattaagagtgtttgtttaaggtgatctcattgatggtagttctcagccgtctcaaaaactgcaagctaatcagtcagacattctttaatgaccccaattttttcactttaattgttaccatgttttctatttttactgatttttgctaaagcatgtaagagtgaatttattatagcagtaatcttgtgtttctcctgatgtgca

SEQ ID NO: 396 - DNA, 331 bp, human
atcaacacatttagcttggcgttttattcattccatcacccttactgtaactactatggcaactcaaataattgtcatgggactatgtccatcttaatgggtctggatggcatggtgctcctcttaagtgtgctggaattctgcattgctgtgtccctctctgcctttggatgtaaagtgctctgttgtacccctggtggggttgtgttaattctgccatcacattctcacatggcagaaacagcatctcccacaccacttaatgaggtttgaggccacccaaagatcaacagacaaatgctccagaaatctatgctgactgtgacacaag

SEQ ID NO: 397 - DNA, 449 bp, human
catctctcaggaggtgggctgtccaccaaaatgagcattcagggctgcgtggcccaaccttccagcttcttgttgaaccacaccagacaaatcgggatcttctctgcgcgtgagaagcgtgatgtgcagcctcctgcctctcagcatgagggaggtggggctgagggcctggagtctctcacttggggggtggggctggcactggccccagcgctgtggtggggagtggtttgcccttcctgctaactctattacccccacgattcttcaccgctgctgaccacccacactcaacctccctctgacctcataacctaatggccttggacaccagattctttcccattctgtccatgaatcatcttccccacacacaatcattcatatctactcacctaacagcaacactggggagagcctggagcatccggacttgccctatgggagag

SEQ ID NO: 398 - DNA, 483 bp, human
tccttgtccacaagcactatggcccccggctcaagtgctgctctggcaaagctccggagccccagccccaaggctttgacaaccaggcgttcctccctgaccacaaggccaactgggcgcccgtccccagccccacgcacgaccccaagcccgcggaggcaccgatgcccgcagagcccgcaccccccggccctgcctccccaggcggtgcccctgagccccccgcagcggcccgagctggcggaagccccacggcggtgaggtccatcctgaccaaggagcggcggccagagggcgggtacaaggctgtctggtttggcgaggacatcgggacggaggcagacgtggtcgttctcaacgcgcccaccctggacgtggatggcgccagtgactccggcagcggcgatgagggcgagggcgcggggaggggtgggggtccctacgatgcgcccggtggtgatgactcctacatctaagtggccc

SEQ ID NO: 399 - DNA, 241 bp, human
accatcgctggtggtatcccagggtccctgctcaagttttctttgaaaaggagggctggaatggtacatcacataggcaagtcctgccctgtatttaggctttgcctgcttggtgtgatgtaagggaaattgaaagacttgcccattcaaaatgatctttaccgtggcctgccccatgcttatggtccccagcatttacagtaacttgtgaatgttaagtatcatctcttatctaaatatt

SEQ ID NO: 400 - DNA, 470 bp, human
gaatactcataattcttatctctataatcaaaagtataatttactgtagaaaaataaagagatgcttgttctgaaagtaagatcagtgaactgcttttcagtctcaatctttgagaattgtaaattcatcaaataattgcttacatagtaaaaatttaaggtattagaaaacctgcataacaaatagtattatatattaaatattttgatatgtaaagctctacacaaagctaaatatagtgtaataatgtttacactaataagcaaatatgttaatcttctcatttttttactgtcatataatcttagtgatatgcctattaatagttttaaataaataaattggctcatctggctttttgaaaattttgaaattcttacagatgttgattaggtatatctacaaattaatttcaattttaaaatgatgatataaaaataaatataagtatttttcttgtgtatgtata

SEQ ID NO: 401 - DNA, 488 bp, human
agtaactttgtttatccctcaagcaaatcctgatgacattgatcctactcctactcctactcctactcctgataaaagtcataattctggagttaatatttctacgctggtattgtctgtgattgggtctgttgtaattgttaactttattttaagtaccaccatttgaaccttaacgaagaaaaaaatcttcaagtagacctagaagagagttttaaaaaacaaaacaatgtaagtaaaggatatttctgaatcttaaaattcatcccatgtgtgatcataaactcataaaaataattttaagatgtcggaaaaggatactttgattaaataaaaacactcatggatatgtaaaaactgtcaagattaaaatttaatagtttcatttatttgttattttatttgtaagaaatagtgatgaacaaagatcctttttcatactgatacctggttgtatattatttgatgcaacagttttctgaaatgat

SEQ ID NO: 402 - DNA, 407 bp, human
ttccacttctattccacggagaaaaaaattgatttgtgtcacagaagtttttatggaaactagcaccatgtctacagaaactgaaccatttgttgaaaataaagcagcattcaagaatgaagctgctgggtttggaggtgtccccacggctctgctagtgcttgctctcctcttctttggtgctgcagctggtcttggattttgctatgtcaaaaggtatgtgaaggccttcccttttacaaacaagaatcagcagaaggaaatgatcgaaaccaaagtagtaaaggaggagaaggccaatgatagcaaccctaatgaggaatcaaagaaaactgataaaaacccagaagagtccaagagtccaagcaaaactaccgtgcgatgcctggaagctgaagtttagatga

SEQ ID NO: 403 - DNA, 120 bp, human
ctcaactcggccatcacatatcgaattaccaaccactcacacttccggatggagggagaggttgtgctgaccaccaccacactggcacaggcgggagccttctacgcagaggttgaggcc

SEQ ID NO: 404 - DNA, 321 bp, human
tccattaccaagagctcatgccacccggttcctgcatgccagaggagcccaagccaaagaggggaagacgatcgtggccccggaaaaggaccgccacccacacttgtgattacgcgggctgcggcaaaacctacacaaagagttcccatctcaaggcacacctgcgaacccacacaggtgagaaaccttaccactgtgactgggacggctgtggatggaaattcgcccgctcagatgaactgaccaggcactaccgtaaacacacggggcaccgcccgttccagtgccaaaaatgcgaccgagcattttccaggtcggacc

SEQ ID NO: 405 - DNA, 531 bp, human
aaaacccattctttcagttacatctactaagagctcgtagttcaataacattccaaagaacgtagtttggaaaacactggcctatcggctgtaataggaagggtctaaagaaaattatttgctgagtcctcataatcaattggctataatcacaatttaataatttatcaatgcaaacagtacattaaaaactgtagcacaatctataatttttaccttcatatgcagagaaattaagaatttacatcttttacaaatatgtgtatctctgaaataatgaaatatattgatgggtgaaataatatttctggtattttcttcattataacacgtggaatgggacagtggctagaggtatggatggagcaaggtcaaatatgagctgatggagctgggcgctaaataagggtataatatgctacttctgtgtatgttgtgtatggtatagtatgctatttctgtgtatgttcaaattgtttatgtatataataaagttttaaaaatttgtatgtacttgacaattccttagaa

SEQ ID NO: 406 - DNA, 533 bp, human
agaggagacactttggcgttcttcaagcagaactgaggctgcgaaaaatccaagtctctacagagacactgatgaagttgaaagggtaattgtttttccatgccaaatgaggaaatcaaattaatgagttgacaaacttttcctgagatgttatttcatctacatttagttaaacaacttagaatgatatctagaaaatatttattttcagccaggaactttagcaaactgtggcaatgatcactgggaatgaaaacgtataatgttctacaatttttgttatcacattggatgactttgaatatagtagtgacacttgggaccaaataaattattttatatgactactagaacaaagttttagtaagctgtctgctaatagagtcagaattcttccagattgtttccagctgtaacaggtattgaatttccaccgtgctcctatgtaacagctgggtggtgtaaaaaatgaacttcaaattatggagtggaagaagcgtaatgttaatatcttgtaaattcgtattccctat

SEQ ID NO: 407 - DNA, 394 bp, human
cattttccaggatctcagtgtgagtatcattgatgcctgggatataacaattgcatatggcacaaataatgtacacccacctcaacatgtagtcggaaatcagattaatatattattaaactatatttgttaaataacacaaaagtctgaaattcattcacttaagtaaaaaaatttattgactgtctactagcaggccagatgctgtgtttggctctgaattcccaactagcaagagcagagaatctacattatggctgatccataagccaccaattcagctaaatgagatgtttctaatctgggcttccacttaaaaataaaactaaaaatactcatgaaaagagcctacccttcttgatcagagccatacccctcttcaccaatgggaagt

SEQ ID NO: 408 - DNA, 478 bp, human
ggtattttagtctagttttatatgaacggttgtatcagggtaaccaactcgatttgggatgaatcttagggcaccaaagactaagacagtatctttaagattgctagggaaaagggccctatgtgtcaggcctctgagcccaagccaagcatcgcatcccctgtgatttgcacgtatacatccagatggcctaaagtaactgaagatccacaaaagaagtaaaaatagccttaactgatgacattccaccattgtgatttgttcctgccccaccctaactgatcaatgtactttgtaatctcccccacccttaagaaggtactttgtaatcttccccacccttaagaaggttctttgtaattctccccacccttgagaatgtactttgtgagatccaccctgcccacaaaacattgctcttaacttcaccgcctaacccaaaacctataagaactaatgataatccatcacccttcgc

SEQ ID NO: 409 - DNA, 487 bp, human
gctggccaagactactgggccgtgctttctggaaaaggcatttcagccacgctgatgatcttctccctcttggagttcttcgtagcttgtgccacagcccattttgccaaccaagcaaacaccacaaccaatatgtctgtcctggttattccaaatatgtatgaaagcaaccctgtgacaccagcgtcttcttcagctcctcccagatgcaacaactactcagctaatgcccctaaatagtaaaagaaaaaggggtatcagtctaatctcatggagaaaaactacttgcaaaaacttcttaagaagatgtcttttattgtctacaatgatttctagtctttaaaaactgtgtttgagatttgtttttaggttggtcgctaatgatggctgtatctcccttcactgtctcttcctacattaccactactacatgctggcaaaggtgaaggatcagaggactgaaaaatgattctgcaactctcttaaa

SEQ ID NO: 410 - DNA, 313 bp, human
agcccaccagcaggagcttggagtttggggagtggggatgagtccgtcaagcacaactgttctctgagtggaaccaaagaagcaaggagctaggacccccagtcctgccccccaggagcacaagcagggtcccctcagtcaaggcagtgggatgggcggctgaggaacggggcaggcaaggtcactgctcagtcacgtccacgggggacgagccgtgggttctgctgagtaggtggagctcattgctttctccaagcttggaactgttttgaaagataacacagagggaaagggagagccacctggtacttgt

SEQ ID NO: 411 - DNA, 325 bp, human
gccactatcttgaagaaggtgcacagtgccctgctcctctttcctatgtccccagaattctcttagggttctcagatgccatgactttcaaggagagagtacggaaccacatcatgcacttggaggaacatttattttgccagtatttttccaaaaatgccctagaaatagcctctgaaattctccaaacacctgtcacagcatatgatctctacagccacacatcaatttggttgttgcgaacagactttgttttggactatcccaaacccgtgatgcccaatatgatcttcattggtggtatcaactgccatcagggaaagcc

SEQ ID NO: 412 - DNA, 284 bp, human
ctttatcatccccacaaacattttgaaactggaatatttgtcttcagaaaatggaaacaagactataaatgataagccctgtccctagcaccacctctcctgtgtgtggaatagaggcccctcgtgctaccaacacttaccctgtgtttaaaaagatcttgtaccaagccaacggcgttcctggctctcctgcccacaggatgaacattttcggcttccttaggagttttgccctaccgtattccaaagcgtgtgctggtttctcatattgtctgtaggctcac

SEQ ID NO: 413 - DNA, 539 bp, human
gtgaggctcagtcagaacctccaccctcccccacaccaaagacaggggcagcgtagtattcaaaccagtattgtggtggggaataattgtatacatgtaaattatcaagccctatgagtggaagaattttttcaaattatttttgtctctctatatattgatttatattatgtataactatctctttatataaactatatataattatatatatataactatataattatatatatataactatatatataactatatatatgtatcccctagtattggatcatgaagagctcttcatgcattctttgcaaaggaggttataaagttacgccctcagaacatttataactataagaatgtgccagttaaagtgctcaacaggaaatatgacagtttaaaagcattgtaaaactcacatagcttacttctctctctaaagtgcaacaaggatgaatagaatgggccaaggtatgacaattaatggttctgcatgacctagccactgctgggggttttcttctataacgttgtccttgtga

SEQ ID NO: 414 - DNA, 511 bp, human
gggacttaacatttcacgttgtatcttacttgcagtgaatgcaagggttacttttctctggggacctcccccatcacccaggttcctactctgggctcccgattcccatggctcccaaaccatgccgcatggtttggttaatgaaacccagtagctaaccccactgtgcttccacatgcctggcctaaaatgggtgatatacaggtcttatatccccatatggaatttatccatcaaccacataaaaacaaacagtgccttctgccctctgcccagatgtgtccagcacgttctcaaagtttccacattagcactccctaaggacgctgggagcctgtcagtttatgatctgacctaggtcccccctttcttctgtcccctgtttttaagtccggatttttacagaaggaactgtctccagacagctcatcaaggaaccaagcaaaggccagatagcctgacagataggctagtggtaattgtgtatatgggcgggacgtgtgtgtcatta

SEQ ID NO: 415 - DNA, 316 bp, human
cctctgtcctcaaatgtccaaaatgttggaggacctctgttcatatcccacgcctgggctcttgccagcagtggagttactgtagagggatgtcccaagcttgttttccaatcagtgttaagctgtttgaaactctcctgtgtctgtgttttgtttgtgcgtgtgtgtgagagcacatcagtgtgtgcaggctgtgtttccccatttctctcctcccttcagacccatcattgagaacaaatgtaagaaatcccttcccaccaccctccctgcctcccaggccctctgcgggggaaacaagatcacccagcatcct

SEQ ID NO: 416 - DNA, 149 bp, human
  misc_feature (55)..(55): n is a, c, g, or t
atattttgtatcatcgtgcctatagccgctgccaccgtgtataaatcctggtgtntgctccttatcctggacatgaatgtattgtacactgacgcgtccccactcctgtacagctgctttgtttctttgcaatgcattgtatggcttta

SEQ ID NO: 417 - DNA, 523 bp, human
  misc_feature (29)..(29): n is a, c, g, or t
atccgacttgaatattcctggacttacanaatgccaagggggtgactggaagttgtggatatcagggtataaattatatccgtgagttgggggagggaagaccagaattcccttgaattgtgtattgatgcaatataagcataaaagatcaccttgtattctctttaccttctaaaagccattattatgatgttagaagaagaggaagaaattcaggtacagaaaacatgtttaaatagcctaaatgatggtgcttggtgagtcttggttctaaaggtaccaaacaaggaagccaaagttttcaaactgctgcatactttgacaaggaaaatctatatttgtcttccgatcaacatttatgacctaagtcaggtaatatacctggtttacttctttagcatttttatgcagacagtctgttatgcactgtggtttcagatgtgcaataatttgtacaatggtttattcccaagtatgccttaagcagaacaaatgtgtttttctatatagttccttgccttaa

SEQ ID NO: 418 - DNA, 576 bp, human
ccaatcctgaaggtactccctgtttgctgcagaatgtcagatattttggatgttgcataagagtcctatttgccccagttaattcaacttttgtctgcctgttttgtggactggctggctctgttagaactctgtccaaaaagtgcatggaatataacttgtaaagcttcccacaattgacaatatatatgcatgtgtttaaaccaaatccagaaagcttaaacaatagagctgcataatagtatttattaaagaatcacaactgtaaacatgagaataacttaaggattctagtttagttttttgtaattgcaaattatatttttgctgctgatatattagaataatttttaaatgtcatcttgaaatagaaatatgtattttaagcactcacgcaaaggtaaatgaacacgttttaaatgtgtgtgttgctaattttttccataagaattgtaaacattgaactgaacaaattacctataatggatttggttaatgacttatgagcaagctggtttggccagacagtatacccaaacttttatataatatacagaaggctatcacacttgtgaa

SEQ ID NO: 419 - DNA, 323 bp, human
tcgactctgctgctcatgggaagaacagaattgctcctgcatgcaactaattcaataaaactgtcttgtgagctgatcgcttggagggtcctctttttatgttgagttgctgcttcccggcatgccttcattttgctatggggggcaggcaggggggatggaaaataagtagaaacaaaaaagcagtggctaagatggtatagggactgtcataccagtgaagaataaaagggtgaagaataaaagggatatgatgacaaggttgatccacttcaagaattgcttgctttcaggaagagagatgtgtttcaacaagccaacta

SEQ ID NO: 420 - DNA, 448 bp, human
aataacgcaaatggcttcctctttcctttttttggaccatctcagtctttatttgtgtaattcattttgaggaaaaaacaactccatgtatttattcaagtgcattaaagtctacaatggaaaaaaagcagtgaagcattagatgctggtaaaagctagaggagacacaatgagcttagtacctccaatttcctttctttcctaccatgtaaccctgctttgggaatatggatgtaaagaagtaacttgtgtctcatgaaaatcagtacaatcacacaaggaggatgaaacgccggaacaaaaatgaggtgtgtagaacagggtcccacaggtttggggacattgagatcacttgtcttgtggtggggaggctgctgaggggtagcaggtccatctccagcagctggtccaacagtcgtatcctggtgaatgtctgttcagctcttct

SEQ ID NO: 421 - DNA, 477 bp, human
tcaaatggaacctgccctctaaagcactttctttcctttacttgcgtggtttcatgtaagctgtgctgtttagaaacaacatctcagactttacaaagaaatgacaaagaaggcaattgcactttttaagggatatcgacaagcagtttctgttttctaaaggacaaaatacagagtgtgtgtcatttttaattagattctttcccctgctgagttggaaattccagtgcagcactgattgaccacagttgccaatctaaaagcacaaagacagaagtaaagctttatgctaattttatttcaatatgatagaaaatttatcttggtatgtccttttttagataactccagcaggaaactgtaactgctatgtctttaggaaaacgtagaagaaagaacattattattctttaattcctacaaggtacttgaaaaccttaagtgaaaaagatttctatctttttatcttggcgcatt

SEQ ID NO: 422 - DNA, 577 bp, human
tgccactaattcattcacactaaggtgtaaatgattgataataggaatgagttacctcttcccacagacatttgtttttaagtatgacagagcagggccttaatcccaagggaaaaggttatggaactggagggggtgagctttctgggtagaaggagacttcctgaatttccttaaaacccagtaagagtaagacctgttgttttggaaggtctgctccaccatctaagagcactgtttttttttttttgttgttgttgttgttttacggtctctgagggaatatagtaaaaatgcatatgcacgtgcaatttgcacggcagcatttcaccgattgtggactgtattggctaatgtgtttcctggtctttagatgcaaaccattaataacactatcttatctcatagttttttcaggggtgcttcttgattagtagggaattttgaacacctctttaaatacagctagaaaataaaaccaatttgtaaagccacatttgcatatgatgccagcctcacgcatttgtatatctccagaaattcaggtatgcctcaccaatttgcccgtctttaataa

SEQ ID NO: 423 - DNA, 544 bp, human
  misc_feature (46)..(46): n is a, c, g, or t
  misc_feature (64)..(64): n is a, c, g, or t
  misc_feature (66)..(66): n is a, c, g, or t
  misc_feature (97)..(97): n is a, c, g, or t
taaactaacccttacattccatgtgatgtgatgtaggcttataagntgctaaaatctataggtntnggaagtgaaagattctattttttcttttttngtacataatgggaatttcattccagattatattttatttacatattaatttcacagaatattaatatttcttaacttcttaaagcatgctagcgttttatgtatatgtacacatatattcagacagggtaattttatctgctgcctaacattgtactaaaatattgctttatctgtttttaattacaaaatgctaatgatttcttaaattatagtttaaagacaattggcccaggaagcaaatcccctgcctttagtatgaaccactataagtaaccttacaaatagagttaatccaagacaatattaacaaactgtgctttgtctttaataaaagggataggattaacaaacatattgatggcataacctattcagctatgtccttatttttgcaataatgtaacctcaaatattggattgttgaaccaacaatggctgtgttaaa

SEQ ID NO: 424 - DNA, 512 bp, human
  misc_feature (79)..(79): n is a, c, g, or t
tttatgatgactcagtggtgccagagtaaagtttctaaaataacattcctctcacttgtaccccactaaaagtattagnctacacattacattgaagttaaacacaaaattatcagtgttttagaaacatgagtccggactgtgtaagtaaaagtacaaacattatttccaccataaagtatgtattgaaatcaagttgtctctgtgtacagaatacatacttattcccatttttaagcatttgcttctgttttccctacctagaatgtcagatgtttttcagttatctccccatttgtcaaagttgacctcaagataacatttttcattaaagcatctgagatctaagaacacaattattattctaacaatgattattagctcattcacttattttgataactaatgatcacagctattatactactttctcgttattttgtgtgcatgcctcatttccctgacttaaacctcactgagagcgcaaaatgcagctttatactttttacttt

SEQ ID NO: 425 - DNA, 517 bp, human
gataacctacatcaaccgagataccaaaatcatcctggagaccaagagcaagaccatttacaagctgaacggtgtgtccgaaagggacctgaagaaatcggtgctgtggctcaaagacagcttgcagtgcacctgtgaggagatgaacgacatcaacgcgccctatctggtcatgggacagaaacagggtggggagctggtgatcacctcggtgaagcggtggcagaaggggcagagagagttcaagcgcatctcccgcagcatccgcaagctgcagtgctagtcccggcatcctgatggctccgacaggcctgctccagagcacggctgaccatttctgctccgggatctcagctcccgttccccaagcacactcctagctgctccagtctcagcctgggcagcttccccctgccttttgcacgtttgcatccccagcatttcctgagttataaggccacaggagtggatagctgttttcacctaaaggaaaagcccacccgaatcttgtagaaat

SEQ ID NO: 426 - DNA, 441 bp, human
ggtgcaactgtgacttgggtctggttggttgttgtttgttgttttgagtcagctgattttcacttcccactgaggttgtcataacatgcaaattgcttcaattttctctgtggcccaaacttgtgggtcacaaaccctgttgagataaagctggctgttatctcaacatcttcatcagctccagactgagactcagtgtctaagtcttacaacaattcatcattttataccttcaatgggaacttaaactgttacatgtatcacattccagctacaatacttccatttattagaagcacattaaccatttctatagcatgatttcttcaagtaaaaggcaaaagatataaattttataattgacttgagtactttaagccttgtttaaaacatttcttacttaacttttgcaaattaaacccattgtagcttacctgtaat

SEQ ID NO: 427 - DNA, 377 bp, human
agggtacgagaacttgccaatgggaaattcatccgagtggcactggcagagaaggataggagtggaatgcccacacagtgaccaacagaactggtctgcgtgcataaccagctgccaccctcaggcctgggccccagagctcagggcacccagtgtcttaaggaaccatttggaggacagtctgagagcaggaacttcaagctgtgattctatctcggctcagacttttggttggaaaaagatcttcatggccccaaatcccctgagacatgccttgtagaatgattttgtgatgttgtgatgcttgtggagcatcgcgtaaggcttcttgcttatttaaactgtgcaaggtaaaaatcaagcctttggagccacag

SEQ ID NO: 428 - DNA, 521 bp, human
  misc_feature (96)..(96): n is a, c, g, or t
  misc_feature (102)..(102): n is a, c, g, or t
  misc_feature (354)..(354): n is a, c, g, or t
gagtccaaatgtcatcagtgctcattttgagataccctgctatcgatggtcgctacaaaccaggaaatactcaagttattatgtgtatacattggntttagntttatgaaacaatttaccttcatgatctcatagttaaaattgtaataaatttaggaatataaaggatcaatatgggaagcaaaatttctaaaggcagtttctgttgttttaattagtatttgtgtagttcaaaccaggaaggatttgactatcattagattttgcttaactttatgaaagctaaaatattctctgttataaaggggcaactccatctggtcctatagcatctttactactgatttttttttngtttaatttgaaaatgcaaagaattgttaaatgttcttaaatgttctcactacaaaaaaagaaaaaagataactacgtgaggtgatggatatgttaattagctggattgtggtaatcattttggaatgtatatgtatatcaaaacatgtagtacaccctaaatatat

SEQ ID NO: 429 - DNA, 423 bp, human
agatacatttttacccaccataaatgttacaatatctgaatatgctttgtcaaactatccctttatgcaatcgtcttcatattgtttttatgattctaatcaagctgtatgtagagactgaatgtgaagtcaagtctgagcacaaaaagataatgcacgatgagattgcctaccattttataggatatttactatgtatttatacgttaagacctctatgaatgaatgtatcagagaatgtctttgtaactgtttaattcaatctgtaataaaaatctaactaactaactcatttatttctattaaaaaggtattgtcctttaggcggggaatgggaatccttgctgcactgttgcagtcattctgaaaggacctttccctgtacttacctttcaacatgcttcaatcttatcaacgctacat

SEQ ID NO: 430 - DNA, 465 bp, human
gaaaaccgatgtgatccttgatcgaaaaaaaaatccagaaccttgggaaactgtggaccctactgtacctcaaaagcttataacaatcaaccaacaatggaaacccattgaagagttgcaaaatgtccaaagggtgaccaaatgacgagccctcgcctctttcttctgaagagtactctataaatctagtggaaacatttctgcacaaactagattctggacaccagtgtgcggaaatgcttctgctacatttttagggtttgtctacattttttgggctctggataaggaattaaaggagtgcagcaataactgcactgtctaaaagtttgtgcttattttcttgtaaatttgaatattgcatattgaaatttttgtttatgatctatgaatgtttttcttaaaatttacaaagctttgtaaattagattttctttaataaaatgccatttgtgcaagatttct

SEQ ID NO: 431 - DNA, 548 bp, human
aattgtgttgtgatgctactcactttgattgcaatgatgatgtccaaggtaagctattaaaaggcaggttacttccaaatcgcactgaaggaaaaggttaagaataatacatgatcacagaaatgcataccactgtctgtaaacccaacaaaattcactgttctcttttggatttatttagcctgatgtatttttaattcaatttttatggtgatgggcaaatcattcttggtaaatgtaaatcaaacatgattgatttaaaacttcatggaatttgtagaaaattatggacatttttggtgagaaagaacaatagtcaaaactcacatggatagagtgtgtttgttttttgccaaaaatgccccagaccttttcccaaacctcaaaaacgtcttggaaaaattgtaaaagtttgataacagaaacatctttaggatatttttgtctgacgtattttgcttctagtatgtgcctactgtgatttttttcatgtggaaaatgcaaaatttgtaacaaaatggttatatggaacatgcctattaaacgaa

SEQ ID NO: 432 - DNA, 494 bp, human
ggcatctggcacgtgcccaataagtcccccatgcagcactggagaaacagctccctgctgaggtaccgcacggacactggcttcctccagacactgggacataatctgtttggcatctaccagaaatatccagtgaaatatggagaaggaaagtgttggactgacaacggcccggtgatccctgtggtctatgattttggcgacgcccagaaaacagcatcttattactcaccctatggccagcgggaattcactgcgggatttgttcagttcagggtatttaataacgagagagcagccaacgccttgtgtgctggaatgagggtcaccggatgtaacactgagcaccactgcattggtggaggaggatactttccagaggccagtccccagcagtgtggagatttttctggttttgattggagtggatatggaactcatgttggttacagcagcagccgtgagataactgaggcagctgtgcttctattcta

SEQ ID NO: 433 - DNA, 443 bp, human
gagaagcacctgttccaactaagactaaagtggccgttgatgagaataaagccaaagaattccttggcagcctgaagcgccagaagcggcagctgtgggaccggactcggcccgaggtgcagcagtggtaccagcagtttctctacatgggctttgacgaagcgaaatttgaagatgacatcacctattggcttaacagagatcgaaatggacatgaatactatggcgattactaccaacgtcactatgatgaagactctgcaattggtccccggagcccctacggctttaggcatggagccagcgtcaactacgatgactactaaccatgacttgccacacgctgtacaagaagcaaatagcgattctcttcatgtatctcctaatgccttacactacttggtttctgatttgctctatttcagcagatctttctacctact

SEQ ID NO: 434 - DNA, 507 bp, human
agctgccgggcaacgtgttgtgtaagtgaacatctgggaggtaaacactacacgtgaagagtggtgaaagggaacattgattactgaagtgccctggagagggaaagcactggtcaacatcacatggacaaatttcattgttttctaaagatggcctggaagtagtctttgccactgcttcctccacaaacagctcttcataacatgggctgcatgaaatcaaagcaaactttcccatttcctaccatatatgaaggtgagaagcagcatgagagtgaagaaccctttatgccagaagagagatgtctacctaggatggcttctccagttaatgtcaaagaggaagtgaaggaacctccagggaccaatattgtgatcttggaatatgcacaccgcctgtctcaggatatcttgtgtgatgccttgcagcaatgggcatgcaataacatcaagtaccatgacattccatacattgagagtgaggggccttgaggctgtaggatgaca

SEQ ID NO: 435 - DNA, 392 bp, human
ggggctatactccatccaaatatgcagtggaaggtttcaatgacagcttaagacgggacatgaaagcttttggtgtgcacgtctcatgcattgaacgtctagacaaactgaaaggcaataaatcctatgtgaacatggacctctctccggtggtagagtgcatggaccacgctctaacaagtctcttccctaagactcattatgccgctggaaaagatgccaaaattttctggatacctctgtctcacatgccagcagctttgcaagactttttattgttgaaacagaaagcagagctggctaatcccaaggcagtgtgactcagctaaccacaaatgtctcctccaggctatgaaattggccgatttcaagaacacatctccttttcaacc

SEQ ID NO: 436 - DNA, 512 bp, human
ggggctatactccatccaaatatgcagtggaaggtttcaatgacagcttaagacgggacctgaaagcttttggtgtgcacgtctcatgcattgaaccaggattgttcaaaacaaacttggcagatccagtaaaggtaattgaaaaaaaactcgccatttgggagcagctgtctccagacatcaaacaacaatatggagaaggttacattgaaaaaagtctagacaaactgaaaggcaataaatcctatgtgaacatggacctctctccggtggtagagtgcatggaccacgctctaacaagtctcttccctaagactcattatgccgctggaaaagatgccaaaattttctggatacctctgtctcacatgccagcagctttgcaagactttttattgttgaaacagaaagcagagctggctaatcccaaggcagtgtgactcagctaaccacaaatgtctcctccaggctatgaaattggccgatttcaagaacacatctccttttcaacc

SEQ ID NO: 437 - DNA, 262 bp, human
tatctgcggccccaggacagaaggtcgccatctcctgctctggaagcagctccaacattgggaataattatgtatcctggtaccaacaactcccaggaacagcccccaaactcctcatttatgacaataataagcgaccctcagggattcctggccgattctctggctccaagtctggcacgtcagccaccctgggcatcaccggactccagactggggacgaggccgattattactgcggaacatgggatagcagcctgag

SEQ ID NO: 438 - DNA, 544 bp, human
ttttatttcaggcttccagctgtccctgtgagttatcctggacatttcgatggtttttggtaaggccaaactctgataagcaaaacagagaatactgacggtatacttaaccatatgtgtaactgatacttggcaccatggaatttttcattgagttatttcctcattcttttaaaaaataagggactataaatcagttatttagtatcttttgtttttgtagctgattccttaactttcttgtatgcctctagtaatttcagagattaaatattgctttaaactgtgatactttgatttgctagattgacaaaactgatactaatataattaagttcatctttgaaatacatctttgtgcgtagagccaaaaaaagagataaaattaataatagttcacttgttatttgagattaatttggcatttgaaatgatcattttattttacaatcatttataatgaatcaatgttccagttagctttaaaaggtatacggtgctaattagtaaaatattgaaggcaatattttactgctagcttgca

SEQ ID NO: 439 - DNA, 380 bp, human
taatcctgacattagctgacttgctagtgagcttgctttaaaaatctacactcttgcattcttaggcatacaggggaaatgttgaaaaggaaggtggaaaaccaagaatttagtttgccaatgattgcctctgattcttgtaagtttgagttccacaagggctaatttattccccttttacttgggttttggggtggtggaaagcgggaaatttgggtgatttgttgattggcaatgaggataaaatgttaatacttttttggggacttaacaactttatcctattctacaagtcagtaaaggaacaattggtactcacctcagtgctgcactcaactatggaaagaggcagagtttgcttgcccaattgccaaactaaa

SEQ ID NO: 440 - DNA, 529 bp, human
attagtgtcattagtgtgttggaagagaaatactattcagtaagcttcgccaaagaaaagtgagtcaaagttaatgtgtgtgtgcatttatatgtaggcagctcgtagaccacattttagccagcaactggtaacaaagagcttagttttccttgtttgaatgctgtagatctgtacctagtacccctcccatctactgatttgtttgtttttgtaaccaaacacattttcagatagaaggagccttaaaaaaaaaaaaatcacattgagtaacttcagtatgaatgaatgagagtgtgtggagctacccctcaccctccacccctttgtgctttttattcccgaattttcccagtctcttaaacagaaaaatgactgatataattatcttttggaaactgagccttaattttttttagagggggaaataagttttccccaactcacacagcataagcaatgtttgacagcaatataatgccgttgtaaactactgagagtattgtatctgttctggtaaccatgtaca

SEQ ID NO: 441 - DNA, 389 bp, human
  misc_feature (74)..(74): n is a, c, g, or t
  misc_feature (181)..(181): n is a, c, g, or t
ttgtttttgacatcagctgtaatcattcctgtgctgtgttttttattacccttggtaggtattagacttgcacnttttttaaaaaaaggtttctgcatcgtggaagcatttgacccagagtggaacgcgtggcctatgcaggtggattccttcaggtctttcctttggttctttgagcatnctttgctttcattcgtctcccgtctttggttctccagttcaaattattgcaaagtaaaggatctttgagtaggttcggtctgaaaggtgtggcctttatatttgatccacacacgttggtcttttaaccgtgctgagcagaaaacaaaacaggttaagaagagccgggtggcagctgacagaggaagccgctcaaataccttcacaat

SEQ ID NO: 442 - DNA, 450 bp, human
ggtcagatggtcaacttttttcagtattatttatagttggcacttgattgcagttctgtgaggcttgagcattcatacacctcacctgccttggcaagcctattttagtgatatggcagcacggatataacactatgcattaaaagcactttttgtaataagtttaatatcctaaaaggaatgccaattaagttttgttaactgtgtcatcaacttatcctagtacctcagtgttcattcctgttacctgcatatcttcttaaaagaaatagctgttattaatgcctttttgttttccattgagtgtacactactgaataagtgtaggagttttatgtttaccatgtgagtcctgcaacactaaagatattttgaatatcagtcatgatggcaatttctgtataaaagagccttaaatggaacattgttttgagatcaaactccccaccc

SEQ ID NO: 443 - DNA, 496 bp, human
gatatctggccatgggtaacctcattgtaactatcatcagaatgggcagagatgatcttgaagtgtcacatacactaaagtccaaacactatgtcagatgggggtaaaatccattaaagaacaggaaaaaataattataagatgataagcaaatgtttcagcccaatgtcaacccagttaaaaaaaaaattaatgctgtgtaaaatggttgaattagtttgcaaactatataaagacatatgcagtaaaaagtctgttaatgcacatcctgtgggaatggagtgttctaaccaattgccttttcttgttatctgagctctcctatattatcatactcagataaccaaattaaaagaattagaatatgatttttaatacacttaacattaaactcttctaactttcttctttctgtgataattcagaagatagttatggatcttcaatgcctctgagtcattgttataaaaaatcagttatcactataccatgctat

SEQ ID NO: 444 - DNA, 538 bp, human
gacctaatacatttcctctgtgtgtgtgtgtaacattccaaatactttttttttcttttccactgtttgtaaggtgcaacaatttaatatttttaagggactttttaagagttccttaagaaccaatttaaaattacttcagtgcaatcctacacagtatcaacattagaattttgatattagtcttatgttatcttccattctatttttatctgctttttgctgctagtttcaaactgccagtatttttccttttgcttttaaaatagttacaatatttttcatgatagccacagtattgccacagtttattataataaagggtttttatttgatttagcgcattcaaagcttttttctatcacttttgtgttcagaatataacctttgtgtgcgtgtatgttgtgtgtgtgcatgtgtggcgtatatgtgtgttacaggttaatgccttcttggaattgtgttaatgttctcttggtttattatgccatcagaatggtaaatgagaacactacaactgtagtcagctcacaatttt

SEQ ID NO: 445 - DNA, 474 bp, human
gagtgagtcccagttacatcaaacagtgacttccagttattccccagtaagtctgagtggttccttcaagctgggtgtctttccagcctttgccagtctagccccagcagggcaccgtgtatgaatgcagtttggtgctgttttagagtatgcctgctccccagccccctgcctggaaccctctgagcaacttgctctgacctataatgtcttaggtgcaacacggaccccaccagagctcttggatacccccctagatccatgtggctttatgtgaggggactgaatgcagacacaccatagcccccttctactactttccctctcgccctgccacctagttccacatggaaccaacaagttgagtgcatccctgttgggtgttttgtgttgagactggctgaaatgaggagactttgaccatgtgacgtgtcaacagactcaaggagacaaccacctcaactgggtcatgtg

SEQ ID NO: 446 - DNA, 414 bp, human
ccccagtctcacctacgaaatatgaagagcaaaagctgattttgcttacttgctaaactgttgggaaagctctgtagagcatggttccagtgaggccaagattgaaatttgatactaaaaaggccacctagctttttgcagataacaaacaagaaagctattccaagactcagatgatgccagctgtctcccacgtgtgtattatggttcaccagggggaactggcaaaagtgtgtgtggggaggggaagggtgtgtgagtggttctgagcaaataactacagggtgcccattaccactcaagaagacacttcacgtattcttgtatcaaattcaataatcttaaacaatttgtgtagaagtccacagacatctttcaaccaccttttaggctgcatatggattgccaagtcag

SEQ ID NO: 447 - DNA, 524 bp, human
tcatgcgtcatccctaaaataataagatacatgggatcaaatagcccttgccttttcaacacaaatcagttggaaaattatggtttgagtcctgttgctgccatggcttctgtttctcagaaatgagtgtgtatgaacataccaatctatgtaataggctacctttttttgtcttctttggaactttgtacacaaaccaagacaatatcagggtgacaggtgaatgaacttaaattctcagtcttgtctattcaccaaaaaagtatactgcctgttttttctttaattattcaaggttgatgacttttaggaacatgttttatactgtattttttaattaaagcaagtgccttgatgtaattccatgtaaatcattgcttaaccctcttatgggatgaggatgagttattaatgtattgcagcctactggaaaggagggggagttggttaatagcagatacttttcttctagaagcttatgttttatgctgtttattatgtaagatcctgtatgtgtgttgaga

SEQ ID NO: 448 - DNA, 426 bp, human
ataatgatccattcgagttctgtgatccttattgttcttaattgtgtttctctacgtattgttacagatgagccatacgtttctttgtatcaatgtagacatgacttcagatacctctgaggacctacccagcagtctaggaccctgggccaagtgctgggactatggtactaaatccagtagatgggctgtgtagcaactctcccagggaacacactagggtacttagggaggtgctttgtggagcatgttgaagctttgagatctgagcaggaggcagtgatgtccctggtctattcagggaaagatttcagtgtgaaatggtaaacatccaattgacaggatttagattttgcttagtttttctgctttttaatgtttctatcccccatctcagtgttttctttatccatcccagtgatgcct

SEQ ID NO: 449 - DNA, 470 bp, human
ataattttacatgatcctcaatatcaactccagtttaaaaagtgttatttttaaaacatttgaaaccaagtactgtttaatttcaatcagaagatgcaaatacatactttgatctatgtttgattttgctaataatatttgaaggagattgcctaccaaggacaaaacaataaatttaaaaatcaaacgatttctccatacgctcatagtcacatatggaattttgagaaaataaagcatgctgtctttaggaatttttatacttctttgtctttcttccttaatatttgcttctagctgctcttggcaatgatgaattgttatgtatgcattaatgttttgcagcccaaaagttgttcacatttttcctatataagatctgtggagtgtgtgtttcaaagagagaactacagaaatgttaaagcaggaaaacctgaatgtgatgtgcacattttcatcccacatggaca

SEQ ID NO: 450 - DNA, 404 bp, human
catttctgtcacatgcacttagttgacattacatctacatatattagctttttcctacatgagccatctatttacttagtaaccagtgttcttaatgaagtatttagtcttgggtttcttgtaaaatttctctgcattccttagacagtgtactatacatgaaatattcttgttgacctagtaatttatattattccatttaattcttaaacctatggcctttttattgagcacactcttaaatcattatttggcttgtaaacattcatctgaattgtggctacaatcctctttaaataatctaggaaaaaagaaagataaagcttacattttcacagttttggctcttaaacacattccacaaatgccattaagaatttattttgttttaggccagtcatggt

SEQ ID NO: 451 - DNA, 522 bp, human
  misc_feature (388)..(388): n is a, c, g, or t
gagcttctagaaatttcacttgcaagtttatttttgcttcctgtgttactgccattcctatttacagtatatttgagtgaatgattatatttttaaaaagttacatggggcttttttggttgtcctaaacttacaaacattccactcattctgtttgtaactgtgattataatttttgtgataatttctggcctgattgaaggaaatttgagaggtctgcatttatatattttaaatagatttgataggtttttaaattgctttttttcataaggtatttataaagttatttggggttgtctgggattgtgtgaaagaaaattagaaccacgctgtatttacatttaccttggtagtttatttgtggatggcagttttctgtagtttnggggactgtggtagctcttggattgttttgcaaattacagctgaaatctgtgtcatggattaaactggcttatgtggctagaataggaagagagaaaaaatgaaatggttgtttactaattttatactcccatt

SEQ ID NO: 452 - DNA, 500 bp, human
  misc_feature (299)..(299): n is a, c, g, or t
  misc_feature (456)..(456): n is a, c, g, or t
ttgtgtgtaatttcatggtggcctagtgttgtggtgcttctggtaatggtaatagaagctcaactatttttttgtggatttcagtttttatcatcagaagtcctagacagtgacatttcttaatggtgggagtccagctcatgcatttctgattatacaaaacagtttgcagtaggttatttgtcatttcagttttttactgaaatttgagctaaacatttttacatgtaaatacttgtatttaccaaagatttaaatcagttgattaattaattaactcaaatactgtgaactatctntaaaacactagaaaaaagaaatgttagtatctcaattacaccaactgtgcaaatgaactttgataaaatagaaataatctacattggcctttgtgaaatctggggaagagctttaggattctagtagatggatactgaatactcaggcccacttaanttattaatgtatacattgtgtttttgtctttatgctatgtacag

SEQ ID NO: 453 - DNA, 512 bp, human
  misc_feature (447)..(448): n is a, c, g, or t
  misc_feature (450)..(455): n is a, c, g, or t
ggcgtgcaattttggtctgcgccacataaccattctgaagcttttaggcgttggagaggaagttgggggagtgttagaactgttcccaattaatgggagctctgttgttgagcgagaagacgtaccagcccatttggtgaaagacattcgtaactattttcaagtgagcccggagtacttctccatgcttctagtcggaaaagacggaaatgtcaaatcctggtatccttccccaatgtggtccatggtgattgtgtacgatttaattgattcgatgcaacttcggagacaggaaatggcgattcagcagtcactggggatgcgctgcccagaagatgagtatgcaggctatggttaccatagttaccaccaaggataccaggatggttaccaggatgactaccgtcatcatgagagttatcaccatggatacccttactgagcagnnannnnnnaccttagactcagccagtttcctctgcagctgctaaaactacatgtggccagctcca

SEQ ID NO: 454 - DNA, 565 bp, human
aactgtgtacttgtctggtcagctgtgtatgatcagttatctacctcagagtctattttcttttgtgctgggacaggttgctggccctccctgtttccacagaccaaatcctcctagctcaggagctagggctaagcagttatttctttcaagtattttttagttcttaaattttatgcttgtatttgatgatagatgtcagtgacatttcatagtttcaaaagtccttgctgctctgagaagtgtagattctagtgaaaattacatagtcataagagaaatgtgtttttgtttttgtttttgtttcatttttttaaagttgtggtattattggttctatgctccctggaatattactgctttgtgaaagtccagactgaacgcagcaccctctgtgtacctagtacagttataaacctgggtctctcactacttgatatttttgcattagttaagacagaaatttgatagctcggttagaggggaggggaaatctgctgctagaaatgtctgaactaagtgccatactcgtctgggtaagatttgggaaacataacctctgtac

SEQ ID NO: 455 - DNA, 575 bp, human
gatgagagcaaaggggtcgcaccatatggaaatgttgaaaactattgtaaagtagtattatgaagtagcttttgtgtcattcatgtcgatgacatgaaagtgaagtaaatttattctatgtaaattcacactaaaaccagtacagtaccataagtagaatacatgtaagaatcacctagtcttcactatattgagtaaatataacatgctaattttacaattaatgaaactaaacttttaaacatctccattatatctacatccttttgaaggtatttatcatagttgccaattttaattttaggattgactttctctttctgaatgacttcataaagtttggtgtgaattttgaagacttgggttactaatgattgtatctttgctagtcaacaacttatgaaatatactcaatgcgtctgatgtgtcattaagtgcagaaataactaagacacaaataacctttgcaaaccttcaagctgtgtaatattccaatgttgtttttttctttgtatatatacttatatcacgtaggatgtaaaaccagtatgaccttgtctagtctccaaacttaa

SEQ ID NO: 456 - DNA, 411 bp, human
  misc_feature (179)..(179): n is a, c, g, or t
  misc_feature (343)..(343): n is a, c, g, or t
gccacacgtccaaaaccaatcaggtcaactcgggcggtgtgctgctgaggttgcaggtgggcgaggaggtgtggctggctgtcaatgactactacgacatggtgggcatccagggctctgacagcgtcttctccggcttcctgctcttccccgactagggcgggcagatgcgctcgagncccacgggccttccacctccctcagcttcctgcatggacccaccttactggccagtctgcatccttgcctagaccattctccccaccagatggacttctcctccagggagcccaccctgacccacccccactgcaccccctccccatgggttctctccttcctntgaacttctttaggagtcactgcttgtgtggttcctgggacacttaaccaatgccttctggtactgcc

SEQ ID NO: 457 - DNA, 550 bp, human
gtatgtttgtaagtgcttctgcacgaatgtttatacatgactgtttccatagtacttatgtttttaaaaatattcagtcatttcctactataatcctcatgtatccatgtaactgactcaaaaatacttcagccacagaaagctaaaactgagcaaatctcattcttcttttccatcccctttgcatgtggctggcatttagtaatgattaataatatggccagctgaataacagaggtttgagacacaattctttctcaaaggagtcagctaagctgggtctacttatggacaaacatctaaatgtgtggaagtatctgatatttgacaatggtaaatttccacttagctagctagcattgtcagacttcaatctcctcatggctctggccgtcctgttttaagcatgataattgttggccacatctcacatagttctcattgagtgagttcataaataaacagggtttttttttttttttaaagagcagccaagcacaaagtgtgactttgttgacattttatgtgactttgtcatatgttcctaacc

SEQ ID NO: 458 - DNA, 353 bp, human
agtgtcaggtgccttgcaggaaaataatctgagtcccaagctagcctgtgctcatccacaatcacaatgaacatgtcaaggaagaatttgcagagactcaagggaagcacaatgggataaggtaatcactttcagtgaaaaactgttttcttgaaaacaggcttggacacaattgaaagctggcttcctgcaaacacaccaagagtctgtaatctagcctatccattatatgtcctttattattcatgatatcctattcttctaccttgttgcctggtaactttttctgaggactgagtttctgcagcgatgtggtgcactcttcctgtgatgaggaaacatctgggccccct

SEQ ID NO: 459 - DNA, 528 bp, human
ctggacacccggttaccaaagtcagcaaagaagatgcggtaatcgccgcctgatctccacatggtgaacacaacactcccaccaacacctccttgactggtcggtcttcagcaccgggggtgggcaggcaggtgttctgtgttgacgagaattgcacaggctaaacacaaacacggaaccagagtgagaacacctcactcacggcagcccaggctgctccctaccaggtgacggagcgcgccggggctgtgggtgccaggggctgagtgctagggactcgtcatgagtggggatccccacgttcctgtcactgctgtcaaacagaaggtaaacagtcttatgaatgtatttccttaggaaaacttgtaaaaacttttattaggatatctatttaatactgaactttggcctactttgtgatagactataaacaaattgaggaaatcactatttctcacttctgtattttctcaaaaataattttgttacagagttcaatatactgtgtaccattgatcttctattgtg

SEQ ID NO: 460 - DNA, 469 bp, human
  misc_feature (99)..(99): n is a, c, g, or t
gaaactgcggatcccaaactgttccctttttcatttcttgaaatgttaccactacagacatttttttaaggtgaataaacagttgtgatgtgctgtacntaaaatcatgtttaatcgtataaggaaacatttcaatacacttatacaggaagaaaactatagatgaagtacatgtgtgtgattcagtctgattcacagaattctgagagtaatatggaataaaacaactccacttagatgataactgaagcatttcctgccttgtgaaaatttggattttaaattgctgttagaatgggaaatttggacactttatatcattgtataatttcagaatttagtttctgtatcttttggaaaacatgattatagcaaaaacatagaaaataatctattactaaaacaccataaatgtaaaactagtatgcttggctgttaactctaaagatgttacttatgtctgttttta

SEQ ID NO: 461 - DNA, 457 bp, human
  misc_feature (151)..(151): n is a, c, g, or t
  misc_feature (155)..(155): n is a, c, g, or t
tgagggtgagcttactcagggcccccagaggaagccctcagcctctgccctccccccacacagggcgggagcccaggcctgttcctggcagctgtggctgcagctgtgctcctgctccctcctggaatgtgcgacaagcccaaatgttccngggnaggcggccggggcagggggcttagaagtgctaatatggttctgtgttttgcctgaaacgataccaggttcccctgaatagcaactttacaaggtccatgtgggagggaccaacccagatgccctgctgagtgtccctgaaaccatggcagctccatctgtcaagatggcaggggccggagtgagggggctgctggcttaacagcaggcatctgggcaggccagtcctcaaagcagctcctgaaggtctgtgttgcactgtcaccagtctcaagctatgcctctaatttcaccagggatattg

SEQ ID NO: 462 - DNA, 139 bp, human
gtgtgatgcatgtgagcgtctctggcacacacacttggacatacagttctgtgtgcgctcattcttattacaggagtgagcaaaggaagcatttaccccgatggttacctagaccacgattatttggattggggggagg

SEQ ID NO: 463 - DNA, 430 bp, human
atccctgtattcatggcttgactttgtgactgctctacactgcatgtctgacattgcagagtgagctatgttgaggtaaactggttggttgtcattattttgcaatcagcctggtctctcccatgaagatgtcgtgtgcataagcacaatcatcactgattagaagatcacagcagaatacccttggattagagagaagttcgtaccttgcatttctctgaattctagtctctcataagcactgctttgctggatgattttcactgctttgtgttaatgactttgagcgatctctcacatgatggggttctttagtacatggtaacagccatgtcatcttacacacctagcattgtgaatgctgtagtgacatcctttataggcaccttacagctcaaaacttttgtttcatttcatgccttacttatca

SEQ ID NO: 464 - DNA, 486 bp, human
agtgccatttattctagtttatcatgttttgcatgtttgaaagtatgaatgtgctctttcctaaaacatggcaaatgaatagatgtagagaataacaatattacttacaagatgaaatgattagattagaagtgtccctttattaaactttgtcagcctgactgggtacaattcttttgttaatttgcagtgtggtttgtatacacgtatacgtgttatcaataataagattttgcaactggatgacacaagattttacttgaacagtgaaggacaaaaatcatgattgtggaagatatttttaaaatctgattttgcagcgatcacttttaaaccctgtagtgatgtaagactaaaatataattgctaagattttgttggttaatgtaaagatatgacttttctgcactgtactctcttcataggattgtaaaggtgttctaatccaattgcatgatgtagtaagcctcttaaatatgtgtgtta

SEQ ID NO: 465 - DNA, 550 bp, human
tcctatgtcttctttcttaaatccagttgctgattttgtaaaatacagttgtgataaagcagcattacgggggggaaaaagctatattccaactggtgttaaatgtattcaacaaaatcttacatcatacagtatttatttcttaattaatagaacttcagtgatatacttggtagatatctcaagccttttgtcttttacacaatggtgctctatcctattgttttcttttcaaagaagcatctgaacacttgcatttctattttcctatccaaaggcatccacatctaagtgtgtttttaaagttgattaaaattatttttctgttaaagcattctgaaagtgtttgtctttacctagaatgatttgtacacactcgtggtcaactgaacatgaatgtcagtagtagtctaattatgggaagggtaaacgtgttagattaaggctcttaaagctctaaaccatataaactatggacttgtatcatgatttaactgttcttagatctttcttacacagtgattcattcctctatttgtacagtggcttt

SEQ ID NO: 466 - DNA, 493 bp, human
tatcaaaggtttgccagccaataaagtgcatcccaagtatacaggggagaaagctagactcctacagggtcctagagtttaagtaatttttttgttattaatataggtaataatttttctaatttttattttttggttccaaatgtaaagctccttgtgtttacctctgtttatgtcattcttgacatgtttatctaaattatgtgtgctctgtgacaggtgaaatgtaaatctgggatccatagtcaagatatcataaggacctacttcccagcctacctttcttcctctacctgataatgataatactcaaaataacaacattcaaaggaaacacaaagaaatcctgctttcacatctcctatttcttgggctccttaataactactgatggtttgttcatgaaaaaaaatttttaaatcaaaagattgtacttggccctgagttgaaaaaatttcaaaaatcaaaagtttgtacttggccctgagttgaa

SEQ ID NO: 467 - DNA, 461 bp, human
cagaaacatgaccctcgctggtcttgggtccacatatcattggactctgggggacacaaagatgcctgtgacactttggtgttgccgagttagtcaacaattattctgggaaaaagcagaattgaattcttctctagatgtcctaccagggttggccaagggccacaaagcaggctaataaattcccacaggatccagacaccaggcaaaattgctctaagaagccagttactgtcatccctctatggttctagaaaaaatagtacaaaaatgacaggtcatcctatgagcgtcatgccaatgaaaccccatcttctggagaagcccttgaatcagaattatcttttttcttgatgtcgtcagatgcagccagtttcttaatttttttaaaaactgtatgtttctgtggtatgtatatttgtacacctaactacctggcacttggaaatcacagcactact

SEQ ID NO: 468 - DNA, 448 bp, human
  misc_feature (28)..(28): n is a, c, g, or t
ttatcctgcgacaacactttcagcagantagcctctccttatctcacagatcacaagcacccctagatagtgtgattctgtcagatagcatttatgcaaaaatctatgaagttaaaagatcgtagaagccaaatgaaatgtacatatctactgactgatgacaagggaatttcattaggaagaaggtaaagaaacatcgttgagtagcctaccttgatttctgtcaagttcataaccagcttcatattttaaaggcttcaggtttgaaattaagtcaactgcatgcagctttgctgataaatgaataattctctttgatgccatttatgagaaaagacttcaatatctgttgcctgtcatatttaagaaaaattactgtttctactctctgtatctgattttaaaagaaaaaactattcatacctggcttccaggtaattgactttga

SEQ ID NO: 469 - DNA, 427 bp, human
gaactgagtcaatatggcaaggtgtatgtgatctgtgggagttatgccatttaacataggaagtgcatgggactttccctctctgcactccagctcttactgtaccattagaagatgcagaattctgttggtgtgcaaaaagtatagccttacattcaagcagaatggatctgaagaaagcagcaatatctgttactagagaacattcccatgtgtttaaactcttcacttcttagatgcatttaaattcttaatgcaaatgacgtagcaatttgaaaacttctccgtattacttgtgtttaaaatgtcttgctttaaatacaaaacaaatggtaaaggggattatcttttgtttagatggttaaatattatttttgccttagatagctttgtaataatttttctccagacagttcaacacttttga

SEQ ID NO: 470 - DNA, 567 bp, human
  misc_feature (246)..(246): n is a, c, g, or t
tactgactgactacaggggctgattgtgaagcacgaggaaccccatgtgtgtggagactgtagggtgagagcacacaattattagcatcatttctgagtgatctcacagattttttttcttgtgtttgttttgctttttgacaactgcttctcccacgttccttgcaattctattctctcaccttcactttactatttgtattcgatggaccaggataattcaggcaaggttaccttgtaaacttnaattggccacacaccatgttgtcacccagctggctatgaagtgaataatggtactgaaagtaaacctgaagacctttctcagatctattttaagtctgagtctgaccaaccatggaaaatattcgacatgaattaatgtagagaactataaagcatttatgacagctccaagaaaaatcatctactctatgcaggagatatgtttagagacctctcagaaaaacttgcctggtttgagggtacacagtaccattttaatcttctgaaaatatctgtattcctgctctttttctgctgtcactgtcaatctgctatattttt

SEQ ID NO: 471 - DNA, 506 bp, human
ttcctgacgggaatgttgtgctataatgaatctgcataacgcttgggattctaggaggaaggaaggttccatggacatgtaagtacagcatattcccctcagtcttctaggagggcagagtgaatcccagaactggtaagattgggaatctgagcattgccactttaatcttagaatatttatcattttgacacatcctgttttttagagaggaaaacaaacacagtttctgcattggtagtgtaaagcataccttgttaggaacgtgttttgtaagacacatttgggttgtcattctagagcatgtcaaactttgtacttcaaaatatatttagtatgattgttagtggtaacatatatcaaggctttgaattaactgttttatttaattttcacaagaagcacttattttagccataggaaaaccaatctgagctacaaatagttctttaaaataagcccaggttatttagctattctagaaagtgccgacttctttcaagaag

SEQ ID NO: 472 - DNA, 488 bp, human
ttaaatattcattccattacatctagactcaccaagaactacatgttatgatgttaagttgaagttgaaacatgatgttttgcattaaatttaagatatgcaaatttatgtagagaaaataaatgttatataccctataatctttcacctaattagtatttaattatatggatttgttttatattataaaagatgttttgattttgtcttttgatattgacaaaattgtttggatatccttatgttctcaagtctgtatctgcctcccctgccttatttcttatgttttgccacagttaacccattgtgcttctttgtaatcaaacagtttgtgggagaatgggcttattgaatgtctaaaaaataagtttaaagtgtttgttaccctaagttttttacatttttaaactctaattacatatgtgaatgttattactctcagtgaattgttattgtttgcaaaaatgcactgggcagtaacattttgt

SEQ ID NO: 473 - DNA, 426 bp, human
  misc_feature (262)..(262): n is a, c, g, or t
  misc_feature (271)..(271): n is a, c, g, or t
gccggactggaaccgtctgagcggcctaacccgcgcccgggtagagacctgcgggggatgacagctgaaccgcctaaaggaggtgaaggctttcgtcacgcaggacattccattctatcacaacctggtgatgaaacacctccctggggccgaccctgagctcgtgctgctgggccgccgctacgaggaactagagcgcatcccactcagtgaaatgacccgcgaagagatcaatgcgctagtgcaggagctcggcttctancgcaaggcngcgcccgacgcgcaggtgccccccgagtacgtgtgggcgcccgcgaagcccccagaggaaacttcggaccacgctgacctgtaggtccgggggcgcggcggagctgggacctacctgcctgagtcctggagacagaatgaagcgctcagcatccc

SEQ ID NO: 474 - DNA, 563 bp, human
  misc_feature (35)..(35): n is a, c, g, or t
  misc_feature (55)..(55): n is a, c, g, or t
  misc_feature (96)..(96): n is a, c, g, or t
aatggcttctatgatcagaactgggaaaacagtgnatcttatggtggaagaggtnctcagcaagtgtacagtatttaccttcctttgtcttacatnggctttttaaattttccattaatttcaacataattatgggaacaagtgtacagaagaattttttttttaagatatgtgagaacttttcatagatgaactttttaacaaatgttttcatttacaggaaattgcaaagaaaattctcaagtgatagtctttttttttaagtgtttcgtaagacaaaaattgaataatgttttttgaagttctggcaagattgaagtctgatattgcagtaatgatatttattaaaaacccataactaccaggaataatgatacctcccaccccttgattcccataacataaaagtgctacttgagagtgggggagaatggcatggtaggctacttttcagggccttgacaagtacatcacccagtggtatcctacatacttctttcaagatcttcaaccatgaggtaaaagagccaagttcaaagaaccctagcacaaatttgctttgg

SEQ ID NO: 475 - DNA, 474 bp, human
  misc_feature (48)..(48): n is a, c, g, or t
  misc_feature (55)..(55): n is a, c, g, or t
ggaacctatcaatttgagtggactttttctttagtagtacaccatttnggttgtngtagtttcaaagtctttctgaagcagatatattgggattggagcggggtggggaaaactgtcactcctttcagaggaaaaggggaggagcatggagaaaaacaaaaattaaaggacttaaagaatggctatacagtgttgagtgttgaggatattaaacatgttatttttcaaacgtatgtaatatatattaaatttataaagcaaatttatgttgtgatcttgcctgaacaaattatattttaatgaaaaaactttctattaatagttcacgcaagagaaaacactttcaacatagtcgaaggcttcaagatctaagtgtatcagacttagggaaaaagtggcacaaccttcgatttaaaattctagtctttaaaatgagtttgtaaataattagctattacgttctattaagttgtt

SEQ ID NO: 476 - DNA, 322 bp, human
agggatctctctacaagagcccctgcccctctgttggaggcacagttttagaataaggaggaggagggagaagagaaaatgtaaaggagggagatctttcccaggccgcaccatttctgtcactcacatggacccaagataaaagaatggccaaaccctcacaacccctgatgtttgaagagttccaagttgaagggaaacaaagaagtgtttgatggtgccagagaggggctgctctccagaaagctaaaatttaatttcttttttcctctgagttctgtacttcaaccagcctacaagctggcacttgctaacaaatcag

SEQ ID NO: 477 - DNA, 409 bp, human
ggtaattatggaaactcctcaaagaggagaaagtaatttttttccagacatttttctcattctgtgtctttcacacactagtttccatagttcgagaattctgttttttaccattgggctgtgaatgttcacaatatcagtcctgttgaattcctatgaggtaatcacaatgtgtatatgttcattttctaggtatgataaaagaatgtatggctttttattctgtggaagtaaaatcctgaacgtttacaacttttccttaacttgtaaataaaaaattgtaagttttttctttttttacagaaaacttagcttgtgtaattctgttagtttcagatttctctcctgtttttgcaaattgtgggaaagattgacaatgcaaatgtgtcaaagacatactgttgggtgc

SEQ ID NO: 478 - DNA, 544 bp, human
acatgggtttatacaagttcctcttgagaaggcaaaaagaccaccatgtgtgagagctctttgacttggccaataggggcctatcttaatgcacttgtttggacacatttctgatcttatttgtaaaggctgcaaaaggagaggatgaaatgctgtaaaagtaggaaatgaagtggaagctggaagaaaatgtaattggtggtacagctatgggccagatggtggaggggagggtggggacccctgccggcaagcagagtgtcacagctggctttcctcacttgggaaaagggtactgccggtctagcagcctcctctgtactcagccaggacacccagcgcgtgggacctgtttgtgtctgttttgcttccttgggaacggcacagtcactcaccctgccatttgcggaaatgacctggtgcactttgactgttaagcaatgcgttattgctgtagtcaaggttagtgcaagcaaggaaacattcccagtaaggtatttgtttccattttctgtctgtgcttctgtcagaaacttgctaggac

SEQ ID NO: 479 - DNA, 479 bp, human
caaacgtcctggactgcacagacctcccactccagaccatccaggcctggttcccaagacccgatccttcccctgcaaccagacagtctacaactgccccctccagcccattttctgccgtgaaaccccagccagccacaccagactctggaaccctttttcgactgccccaactcttggacaccaggccaactagaacacccaacaccaaactgtacagactctcccaccccaacctccccagactctgcacggatgtcctaggccccctccccaactctaaccagaccccatccccctaagtccctttgtcttgacccccaagtcttcaaccagatatcctcggcaacccacctcccaccctcctcctcttctccttcaagacccaactgagcacccgctctgattccccacagcctttctccctgccaccactcccttagtctttcccaggcttactctcccaataaatgtgctag

SEQ ID NO: 480 - DNA, 493 bp, human
ggtggagttggtggtcttaatttggagatgcaggggcaacctgtgaccctttgaggcaagagccctgcacccagctgtcccgtgcagccgtgggcaggggctgcacacggaggggcaggcgggccagttcagggtccgtgccaggccctcctcagtgccctgtgaaggcctcctgtcctccgtgcggctgggcaccagcaccagggagtttctatggcaaccttagtgattattaaggaacactgtcagttttatgaacatatgctcaaatgaaattctactttaggaggaaaggattggaacagcatgtcacaaggctgttaattaacagagagaccttattggatggagatcacatctgttaaatagaatacctcaactctacgttgttttcttggagataaataatagtttcaagtttttgtttgtttgttttacctaattacctgaaagcaaataccaaaggctgatgtctgtatatggggcaaagggt

SEQ ID NO: 481 - DNA, 483 bp, human
atttttccatcttattcaagtcagagcacttttttttaaatagcaatacaacaggcaagagaatactcaaaaatatttaaaactgtattgataccaagagtatgttttaaatattttctaataaatacttgagcggtttttgtctggcaggcttccaaatttgccaaaattaagcgttcagtattttcaacacatacgctttttactggtttatactgaactatctgatgagaattcctgtgttcccaaagcaactgatgtttacaggtcttgtgtttctcctcctcctttctaaggatgagggaatccacaacagactttctctagaaaacactaatgatggacaactttttggtgtcatcaatgagttggctactaccttgatgtaaaaatttgtaaggaaaattttcaccatttcgagtgtcaagtgtatttttaactgtctggtttgtacttttatgacttttgtactaccaaagcgga

SEQ ID NO: 482 - DNA, 408 bp, human
agtgatctctggaagcgctaaagctaaaatttctgttcttgaaacacttcagctttgcaactaaaatattacagattaataataaattaaaccaaccaatgataaacactactcagtccaccaacaacaaacgtgtttgaattcaccttaccaatattaatcccagcgtgtgtaaaacagaacagtaactctatgtgaccccagataacattttgtaacattgtgcttccttgtagtttgtaatgtgagttcaatcagtatttatgttgaaatttctaacattaaatctagtctctatcctgttaatttaatttttaaatgctttatccatttgtgcaaaggtaaacgcagattgtatcttttttaatggtacggcataaaaagtaaccctcaagtgaagtgtctcta

SEQ ID NO: 483 - DNA, 489 bp, human
tgacgagacctcttcaaaaacccacagtaaaactcccctccctccagttggccaccagtctgccaccaaacatgaacaaattctgctgctaatcggtttcccttgtgatctggttcctgaggtcttcggatctgtgcaatgaattatttattgttttattaaaccgacagtggtgtcccagagaggaaccataaataaaatggaaatctggtgctgtgataaagtaataactagcattaatgagacctggttttcctttcagaaagtccagtatacctgtaacaaaggttaaagcaatttatatttaatttgcattctgatgttaacatttaaacagcaattctaacaaaaatgcatcgagtctaattcttacctctatcaaaaaacaactgtataaatttatgaccaacattaaaacaaaaacaaaatgtaaattttctctttagaaatgattaactggaaatgagtgagacagcaccttgtgggttt

SEQ ID NO: 484 - DNA, 460 bp, human
  misc_feature (101)..(101): n is a, c, g, or t
  misc_feature (129)..(129): n is a, c, g, or t
  misc_feature (131)..(133): n is a, c, g, or t
tgctcgcattgatcatcctaatcatcttattcagcctatcccagagaaaacggcacagggaacagtatgatgtgcctcaagagtggcgaaaggaaggcacncctggcatcttccagaagacggccatcngnnnagaccagaatctgagggagagcagattcggccttgagaacgcctacaacaacttccggcccaccctggagactgttgactctggcacagagctccacatccagaggccggagatggtagcatccactgtgtgagccaacgggggcctcccaccctcatctagctctgttcaggagagctgcaaacacagagcccaccacaagcctccggggcgggtcaagaggagaccgaagtcaggccctgaagccggtcctgctctgagctgacagacttggccagtcccctgcctgtgctcctgctggggaaggctgggggctgtaagcctctc

SEQ ID NO: 485 - DNA, 560 bp, human
  misc_feature (144)..(144): n is a, c, g, or t
gctgattctaattgtgtgtaggtcttgaggattaagcacacaaatttcacaaacttctgtttgagtaaacaaactcagccttctgtaaatatacatgcaagtttggaaacagtaatactgtacctataaatatatgctgtctgntttgtgtacagtatgtaaaaactccttttctgccacactaaaaatgcaagccatttatgggaatcctaaaactagtattgaactaaaactttgctaatgatctttattagaggatcgtccaacttttcacttaccttgggttttcttttcaattcactcttacactagtctgcttatttccagctgtttattttattgagtcctgaatttaaaaaaaaaatattttgattcattttgtaaatacaagctgtacaaaaaagagagatttaatgttgtcttttaaatactccaattttcattctaatatgaatgttgttatattgtacttagaaactgtacctttaatattacattacctttattaaaagtgcattgaacacatcaattttagatgtgctttatgtactgttatccta

SEQ ID NO: 486 - DNA, 212 bp, human
taagcattcagtgagctgccaattttgattttgtgttgctctttacccaaattattttttctttgtttttctttttttgggggaggaggggaaaaaagcagcaatactgtgtttggaaattatactctgtatctggttttcctgtgtatgttaaccacttaaatgttattatcctgctttggttttagagtgattgtgaggcattcaatgca

SEQ ID NO: 487 - DNA, 407 bp, human
  misc_feature (75)..(75): n is a, c, g, or t
agtgcctaaatcttgtttacctatcactttaaaaaaataattgaagtgtaagctaaataaaatgcttggagtttngcctgggctagtgagagttggtgcaaattcttgtgtgtgtttgcataggaaggtgagatgaccatctactaaagaggaagtagctaaatacagatctgtgggtgtttttaaaaaaactcaacctatctggtgttttattttaatggataaaaatgtaatttttctaaggtagcaacttatttccaaattaatatagatgaaaaatagataccaattagactaaattgaaagctttttgttctatatttgcatagcctttgaaatatttcttagtgcctaggaggtctggggattcctctttcgtggtggtcactaaccttacttgatgcaga

SEQ ID NO: 488 - DNA, 553 bp, human
  misc_feature (105)..(105): n is a, c, g, or t
  misc_feature (136)..(136): n is a, c, g, or t
  misc_feature (142)..(142): n is a, c, g, or t
  misc_feature (155)..(156): n is a, c, g, or t
  misc_feature (481)..(481): n is a, c, g, or t
aacaccaattatgcaaactacttttttttccagcagaaagggagctgacatgatcaaatccatgttttcaaatgaactgaaaaaggcatccagcaccacttatancacatttatttcagttccaggctgacagccntggggnatctagcaggatnnataattgtcatctggtgagagttgggactttgctcaattttaataccaatctctcctgattgtaattacctccactactttcatgatccccctacaatatttttttaaatgatatatttattcactgaatcaaatgtcattaatgagttatcttctgtggaggatgactgttctctttgttaatgttcacaatcaagatcttgggctgagaagaggcccttcacccaaggagtttgaagtatcacagcgtgtgggaaggtgggaaccaggatacccattcatttccaaccgagacacagagaagtgagtcacagaatttgagccngctctcttgactgcccagccagagacactgatttctgtaacctcttcacttgatcctgcctcttaagcatta

SEQ ID NO: 489 - DNA, 421 bp, human
tcatttctagagtacctgagccaaacaaatacacaacggaagctgcagctgtatcatcactagcaatttgctcatcattatttactacctttgaacctaaggtttcctgcctatgcttttgaaagcaaaaatcagtctcctttgcatgaaaaagagccttagatttttaaacatgttagttaccagaatgctaaaataccagttgattacccaaattattttggaaatctatccataatggaagtctacaacaaacacataaaacagattacactaagagctgagaaattcaaaggaactgaagattctgagagataaactgttcaagtcttagcaatgatactgcacttctctttgacaggttctgggcttaagttagaggccctactggttccaaaccatattccactgactttgcaag

SEQ ID NO: 490 - DNA, 505 bp, human
cctctgagtgttttgtgggttctgggtgttttgtacattttagccaagctaaccacttgtctgcaagtactgactttcctatgaattctttgaagattattgagtcagaaaggaaaaatatagccccaaattcccaggcttttaatgcattacattaactgcctattgaaatgagaagttcttcacaaacttgtatacccactaacaagattgcacataaacatgcattaaagtatatactaagaaaccctctgtccaacggctcatgcatatgaagtccgaacatgggagtttgccaattgcattcatcaagtcgttttgcggagtcagatccctgatggaagagctcacaggctctgccttccaagtcctgggttcctaactggtgaccttagcctggggtctgtggggagaccaaccctggcttccaagaaaaccacattccatggactatcagaaatagacacagatttgggtgacaaagctggctctgtatttgcatttt

SEQ ID NO: 491 - DNA, 497 bp, human
  misc_feature (48)..(48): n is a, c, g, or t
  misc_feature (84)..(84): n is a, c, g, or t
  misc_feature (86)..(87): n is a, c, g, or t
  misc_feature (116)..(116): n is a, c, g, or t
  misc_feature (348)..(348): n is a, c, g, or t
gcaagcgcgtgctgagcaagctgcagtctccatcgcgggcccgcgggncagggggcagtcccggggggctgcagaagcggcacncnngcgtcaccgtcaagtatgaccggcggganctgcagcggcggctggacgtggagaagtggatcgacgggcgcctggaggagctgtaccgcggcatggaggcagacatgcccgatgagatcaacattgatgaattgttggagttagagagtgaagaggagagaagccggaaaatccagggactcctgaagtcatgtgggaaacctgtcgaggacttcatccaggagctgctggcaaagcttcaaggcctccacaggcagcccngcctccgccagccaagcccctcccacgacggcagcctcagccccctccaggaccgggcccggactgctcacccctgaccctcttgcactctccctgccccccggacgccgcccagcttgcttgtgtataagttgtatttaatggttctg

SEQ ID NO: 492 - DNA, 540 bp, human
  misc_feature (380)..(380): n is a, c, g, or t
cactgttccatggtcagcaagtcatatttcataatgtggattttccaaaataattattgaatacagctattctatggctacttttagtgtttttgtggtatgtggtgtgggagtgtttatggaattaccagtatcttaaattttcaaaggaaccttggaagtctatcactctaaatgaaagtctgtcactctacatgaattatgtgctcaaatttgaccaactcagtttaagacacaaaacagtaatttgaagaaggaaaaatgaagagagtttctagtttaatgggttaaatttttgttgttgcaatagtaagtttagtcttcttataatatttctaaatgaaaaatcataggtatttgttaccatgtgtgaagattantttgttaaaagcaaaagtggtcgtgtgatatgctaaatgttaattactgattttatatgtttaaatcacgccaaacaaattatgtctgtgccatccagggtctgttgttaatctttttctgagtacttggattgggataaagggcttgtactatgcactt

SEQ ID NO: 493 - DNA, 569 bp, human
  misc_feature (79)..(79): n is a, c, g, or t
  misc_feature (286)..(286): n is a, c, g, or t
acacttatagtctactgcccatgtaaggatcagctccggctaagaggccaaagatgggtgacatcgttatgctctgccntttattttttctttcttacccacttagcttcctaattggaggaaggaggcgtggtaaaggtatatgaagactatggtttaattagaccagaaaacactgtcataatctctggggtcatcagaatgtccagttttgtctttgggccaagataagggcagtgggatttatgatgtgttgtttatagtctgaaactactctggtgatcanccagggtcagtttctttaatgatggtttccaactggcctaatacattaagtaagactggctgataacatgaccagacagacataaagaccctgttgggaatgacattgaactctcaaagtcaagatttcttacacaaatctatcagctggagaaaatgaaggcagtgtggtatatgtgtgcaaataaggacattatgaagcttaaatatggaatgtctcttggacccccgatgtcatctgtattctctttttcttcttgtactatatcctttgcctgtaaata

SEQ ID NO: 494 - DNA, 326 bp, human
tcccagtgcgaactctgctgtgagtgtgtgcggggaggcgcgcccgcgctgagtcggcggcgggtagccactccatgcccttgtccgatggtttgcaactccgattttgcacaccgctccaccgtgccccccagcgcacacccattcacactcacgccaacactctcgctgaacacttttataattgttaggcgtggccgttgggactttgggcgcagcgcggctgctactgcgtctggaggattgatatttatttttgcattgcgatggctgaaggcatttatttaacgatctttttacctggatatgtctgtgaggctcctgaa

SEQ ID NO: 495 - DNA, 548 bp, human
gtgaagtaccatagaccatattgtctagatgggacaaaataataaattttgactaagaacttactgtttagagagaaaaaatagtaaataatgttcaaaatgagaagacaataactgacaagtctgtgatttttgggttagatgagaccacttttgatttttgaggaataaaaggagttagttttttggtcctgatgtgtaaaatgattcatggggtttcacatacagacaagtagttttcttttcagcaaatatttaacatatctctcaatatatttttcaccagtttttggtaaatatgcattgctataggtataccaagttttttttgttatgtttagtaactaatttcattttttctgtatatgtaaatatagctattggaccctgcattgaaaaccttcttagtactaacaacactccttttattattaccttcatagttactttaaatgacatatttatttcgagttttacttctggctgagttgcaaagtctatgagaatgttacctcctgtctcagaagcttgttagttattgtgccacc

SEQ ID NO: 496 - DNA, 476 bp, human
acatctgctagaaccttttgccttaactattcaccaatatatgctaatattcataaatatggattgactgtttacaaacattagaatcttgtcttggttccattttgatggctaatatttgttatcttaattaagactatttctgaggtcatgattacttgaaaatattgactaaaactgggtccttagaaattccaggtggagctgatttacctatgactgaggggaaaaaaaaatcaaattttactgataatagtaatgctccaaatgaattaatgacacatctgttcaataaataaagagcttaaatatacaaaacataagaaatctgggcaacaaaacttgtggtctttacttttgaatagctacccaagaaaaggttttaaaggtaaaagttatgagtaatgtcatcacaataagctcttgtttaaaattcttttcttttatgtataattaggtttatgtttcatgtcttt

SEQ ID NO: 497 - DNA, 414 bp, human
atcccatgggcaataaagcgcattcaatgtgtttataagccaaacagtcactttgtttaagcaaacacaagtacaaagtaaaatagaaccacaaaataatgaactgcatgttcataacatacaaaaatcgccgcctactcagtaggtaactacaacattccaactcctgaatatatttataaatttacattttcagttaaaaaaatagacttttgagagttcagattttgttttagattttgttttcttacattctggagaaccgaagctcagctcagccctcttccttattttgctcccaaagcctcccccaaatcatcactccctgccccccttaaggctagaggtgagcatgtccctcacaattgcacatgtcaagccatcagcaaggcgcatcacacaaaaggcaccaag

SEQ ID NO: 498 - DNA, 309 bp, human
  misc_feature (61)..(61): n is a, c, g, or t
cagtgagtcgaggttgcgctactgcactccagcctggacaacagagggagactctgtctcnaaaaaaaaaaaaacctacagctgttcaaggaccagctgacaggtcaagtgtggccttttctggtctttgaacacatcatagaaagtgacaaatgctgcaaagccatgaagaacatgaactataaacgggtagactaactgcccagcttagacacttatctatgccacaaaacagctgaatttgtcacatttatatattgcaatatgggaagtattgagatcaaaacaggattccattgacctaattat

SEQ ID NO: 499 - DNA, 233 bp, human
gccatcacagttgcgattccatgagtagctgctttatgactgctttttgtactatctggatgtgcccagagttacttctgtacaagctctgtatctatgtccgttgagaacattattttaacaagaagaacaccaacagtagcatgaaatataatactgttttataattctaaagctgctgttaatttatgaagtacataataatctaatgtaaactgcagaagtcagagcaa

SEQ ID NO: 500 - DNA, 420 bp, human
  misc_feature (71)..(71): n is a, c, g, or t
gcaaccattggggaatgaccttttcatttcagaagtggatgaggaaggtggtgtgagcatcaggtatattnctggaccatttcaagtgctggtgagaagaaaggaactctttgcctgaactgggcttggttttccaagtgctgctttggaaatgaagacccagagatgcagagcttatggtagttcataaatcttcatgttctattatctttcatctgccaataaagttcattttcaataatgtccaccattgctgtgcccagaataaccacaggcaaacatcaaaacaatacgcataagttagacaagattaaatcttgtctgatatctgcacaaacagatatgcaccatgttggaaacatgtgttttcctagtcccatccaggcttcccacaagaaagccatgatgtgggtctaaacc

SEQ ID NO: 501 - DNA, 390 bp, human
  misc_feature (96)..(96): n is a, c, g, or t
attgtttcccatagcagaatgtcaatattcacagtacatttctgtaaagagcaaaccaatataatgttttgagtgttgaaaaaaattccagatttntgaagaattagacaactcttcatctaccttatttctagttcacacagttatctcaaattccactgaaactaatgggatactgtcttgtgtagatgccagttgagtttataatgtgacctagtaaagctgtcttttttgttgtgttgtatgagtgtcggatcatgcttttaggaatacttttattaaaatggtgtgcattcatgcaaaaggccaactggcttttgtgaacaatagatcttttctcccctttattttgttctcttgacacttttgtgaaaattacctagcctgata

SEQ ID NO: 502 - DNA, 460 bp, human
  misc_feature (258)..(258): n is a, c, g, or t
aaagccttccctaaaaagagagcatgccagtcatagagacactaatttggcacttttcctccttcttaggtttaaatgttataccaatgcatgtgtttgaaatatccattaagatgatttaaaatttgtcttgtattttgagtttcttaaatgcgtggattcttgttcatttatctctgaatatgtctcttttatattttggcacaattatacacattggaaagggccaacctattagggctcaagtatgtatatgcnaaaaaaaaaagttatatcaaacaggcacagttattacaactagagagaaattccagaaatatttgtttttttaagagaaagtaaattttcacattagatttctattcaaagtactaatatctacatggtccacacttttctattttaaaaaattgtgttctcttgttaaatagattaacatttccacttctgtttatcacaa

SEQ ID NO: 503 - DNA, 514 bp, human
  misc_feature (285)..(285): n is a, c, g, or t
  misc_feature (469)..(469): n is a, c, g, or t
atctttccatattgaggacagcctctgcatataaaaatgttcattgggagaaaaattatttttaaaactaaatttttatagtagtcctccattactggcaattaaggagcaacccacaaaatgtcattatgtggttctttgattaggcataaaaagtattgcaaacagcttttctattcctttggaatatgaatctttttaatgtctaaagttaacctgcaacattttctagtaggacaagtcaatgtaaattaggtaatttttgcctgttttataccttctgtngcattttgttcaaatgaaaacattttttaaaacatgattgaatttttacttctcagataattggtttgagtgtggaatttcttttttaacactttgatatgtaagttagtgtaaattaatacttttataacactactaggctttatatttttatggaatgttttaaaatttgaaatttttgaantgttaatttagcagtactttatcattatgtgccaatactgacaag

SEQ ID NO: 504 - DNA, 445 bp, human
tttactccagtcattcgatgttgctgagatttacatatgactcttgtcaacatctcatcttttgacccaatcttattcatttaataagaggtctcattcatttgcatggaaaaatgctcattgtatattgcaaagtgaaaataacgagttgcaaaacagtgtatacatatatgtgtgtatatatgtacactttatttgtacatttctatgtgacataatgcaaaggaaagtgtctgattttattatacaccaaaggttaacagtgaatctctgtgtgatctctttttttttctttttgcctatctgcatcttctcacttgccaaaaaatgaatatatgtttatgtgtgtatattacttgtgtcacaaaaaaccctaaagtagacagtaaaagaacttgtcaatcgcctttggaaggcaatgaaacacttaataaactctcaataa

SEQ ID NO: 505 - DNA, 514 bp, human
  misc_feature (26)..(26): n is a, c, g, or t
  misc_feature (128)..(128): n is a, c, g, or t
agccaagaactgaccggggccagggnctgccatggtctccttgcctgctccaaggcacaggatacagtgggaatcttgagactctttggccatttcccatggctcagactaagctccaagcccttcangagttccaagggaacacttgaaccatggacaagactctctcaagatggcaaatggctaattgaggttctgaagttcttcagtacattgctgtaggtcctgaggccagggatttttaattaaatggggtgatgggtggccaataccacaattcctgctgaaaaacactcttccagtccaaaagcttcttgatacagaaaaaagagcctggatttacagaaacatatagatctggtttgaattccagatcgagtttacagttgtgaaatcttgaaggtattacttaacttcactacagattgtctagaagacctttctaggagttatctgattctagaagggtctatacttgtccttgtctttaagctatttgacaactctacgtgtt

SEQ ID NO: 506 - DNA, 432 bp, human
  misc_feature (197)..(197): n is a, c, g, or t
  misc_feature (268)..(268): n is a, c, g, or t
  misc_feature (357)..(357): n is a, c, g, or t
  misc_feature (364)..(365): n is a, c, g, or t
gggctgttccagagattcgatcctcttaaggcattatcagtgagcaaatgtgaaggaaatggtgtctggaagaaagttctggttcacatgccttgtagctaagtctttctgcaaacaacctcccttccccccgtcgagtcatttggtgactttgatggggggatttctggttatgtcaaggctctggagacaggaanggcctttggccgccttgggtagttgacctgccttttctgactccgggacgagccagtcctaggctgcctcngggagcacttgaggtatcccgcaggccatgaggacccactgggcagctcctggacagcctcttggctccagcccccacccgaaagtggnacactgnntccgccctggccacctggggactggcactgtggtgcacagtggcccaatgtggccaacggaagtttt

SEQ ID NO: 507 - DNA, 225 bp, human
  misc_feature (35)..(36): n is a, c, g, or t
  misc_feature (38)..(43): n is a, c, g, or t
cacaggtgtagcactcccaaagcaagactccaganngnnnnnnacctcatgcctggcacctgaggtacccagcagcctcctgtctcccctttcagccttcacagcagtgagctgcaatgttggagggcttcatctcgggctgcaaggaccctgggaaagttccagaactccacgtccttgtctcaattgtgccatcaactttcagagctatcatgagccaacctc

SEQ ID NO: 508 - DNA, 224 bp, human
  misc_feature (59)..(59): n is a, c, g, or t
caccatgaccggtcacagctacaaatccagagaccatcaatcctgctagagtgcagggnggcaagcacccaagggtggctgaccaagactgcagagtctcctccatcttcaggtccattcagcctcctggcatttaactaccagcatccagtggtccccaaggaatcccttcctagcctcctgacatgagtctgctggaaagagcatccaaacaaacaagtaat

SEQ ID NO: 509 - DNA, 177 bp, human
ggttagattccgttttgactgcttggtatctcattgtaagcatttcccagggacactacacagtctttataattatgatcactacatcttttggataacttaggcttttgcactattttgtcacgatgcctcaaacttttctatcattgtctgtaattactcctataatcttttatt

SEQ ID NO: 510 - DNA, 498 bp, human
aatatctcaagatttctaggcattttcctatgtctaatagtaggatgttctctctgaatttatttattacaaaaataacattacaacaataaagcaaaataatatttaaaagaaagaaaaaacaaacagtcgcaataccagaacaccattgtagtcgtttgtgtatgtgtgacttctttactgtgttcatgcagagacaagctttgtaatacctttgtaactactgtacaggtgcgtggttagattccgttttgactgcttggtatctcattgtaagcatttcccagggacactacacagtctttataattatgatcactacatcttttggataacttaggcttttgcactattttgtcacgatgcctcaaacttttctatcattgtctgtaattactcctataatcttttattataaaatattgaaacaaagtcacatcactaaaaacactgtgagtataatttattagtaagtgtagactgtgtatcattagcaaa

SEQ ID NO: 511 - DNA, 453 bp, human
  misc_feature (348)..(348): n is a, c, g, or t
ggagctgcttcaagaagaaacccggcagaagctcaacgtgtctacgaagctgcgccagctggaggaggagcggaacagcctgcaagaccagctggacgaggagatggaggccaagcagaacctggagcgccacatctccactctcaacatccaggtgcctgccccgtgtccttgcttccttcatgggtcctctcaacttctctgcgctgagatcccccgcaggcagatcgcggtggagtgttggtgcgatggtgcttgaccccccagcttcccctgctattgggtttctccaacgaggagacatggtcttcgcttctcagagtctgtggggccagggacaggggccantcatggtccccctctcaccctaccctggacgctgtccttgtagctctccgactcgaagaagaagctgcaggactttgccagcaccgtggaagctctggaagaggg

SEQ ID NO: 512 - DNA, 447 bp, human
  misc_feature (49)..(49): n is a, c, g, or t
  misc_feature (153)..(153): n is a, c, g, or t
  misc_feature (177)..(177): n is a, c, g, or t
tgttacctttcagttacatgcctttgatcctaaaattctctacttttgntgccttatcagttctttgcaatctgcctgtggttatcagcacttaaagcacaattttgaaggggaaaaaaatgataatcaccttagtcccaaagaaataatttntgtcaaactgccttattagtattnaaaacagacacactgaatgaagtagcatgatacgcatatatcctactcagtatcattggccttttatcaaatggggaaactatacttttgtattacatagttttagaaatcgaaagttagagactctttataagtaatgtcaaggaacagtaatttaaaaacaaagttctaacaaatatattgtttgcttaatcacaatgccctcaacttgtatttgaataactaaataggacatgtcttccttggagctgtgggcattagttcagaagc

SEQ ID NO: 513 - DNA, 469 bp, human
gtttacacctatactgcataatccaacaattttaatttcagttgaagacatgttactaatataactattattaaaagagtagaggatgtgtaattaaccatatcttctaaaacatggttactaaaagaatatgtaacatcaatattgaccttggtttcttacacaagtgttgctaactcaatagtgaaggagacactattaaattttctgaacccatgagagatactagagatggggagtggaaagtgtttggttcagggatatctgaagaacagaagggcagagatttcttaagtgacgcctcatctacaagctggaaattcctaaaaacaagtagaaagcttataaacaacaggtgatacactcacctcactggttttagtaaattaccaatacagaaagtatccctagtcttaaaaacaagtggaaaatttgaactgattagtcatattcctttgattacactgtt

SEQ ID NO: 514 - DNA, 236 bp, human
  misc_feature (135)..(135): n is a, c, g, or t
cagtcagagtggacaaacctctgtgggaggctctactgcactgagatgcagctcttccgagggggctcctaagccagtgtacaactgggtgcgtcttggaacttttcctacaccttctcctggcagcatggttcnagatgaggtgtctggccagctcattctcaccaacctctccctgacctcctcgggcacctaccgctgtgtggccaccaaccagatgggcagtgcatcctgtg

SEQ ID NO: 515 - DNA, 488 bp, human
gcctcctcagacactctcaagaggatggggagatgacatcacttgggtacaaacttatgaagaaggtctcttttatgctcaaaaaagtaagaagccattaatggttattcatcacctggaggattgtcaatactctcaagcactaaagaaagtatttgcccaaaatgaagaaatacaagaaatggctcagaataagttcatcatgctaaaccttatgcatgaaaccactgataagaatttatcacctgatgggcaatatgtgcctagaatcatgtttgtagacccttctttaacagttagagctgacatagctggaagatactctaacagattgtacacatatgagcctcgggatttacccctattgatagaaaacatgaagaaagcattaagacttattcagtcagagctataagagatgatagaaaaaagccttcacttcaaagaagtcaaatttcatgaagaaaacctctggcacattgacaaat

SEQ ID NO: 516 - DNA, 452 bp, human
  misc_feature (34)..(34): n is a, c, g, or t
aaggttatagccacattcaacttgaatggtcccnaatgttaaatttaccatgaatactgtgtcttacaatgctgagctctcgactagactaaccagaataggaacacagtctcaaaggataccataagacaaacagtatttagacatataaaaaactacagagatgataacttgaagaataaataacgtgaaaaactactatattcttgaatgggagtagaatgtaagctctttgagggcagggactttgactgtcttgttcattgctatatgataatgcctggcccaacagcaggcattctgtagtgtctgttgaacaaacatctaaaatctggcaaagtgaacaatggttcctgaggataagtatttaaataacaaaattaactttcactacaaaagctgccaacttacctttatcttctggtgctatcccctgagctttcttaagatca

SEQ ID NO: 517 - DNA, 399 bp, human
  misc_feature (119)..(119): n is a, c, g, or t
gaatgcatgatctcaagccttaactactataatctttttctgcccctcagaaattgaataacctaaccaagatgcctttaggggatgccctaagtaaatgtaatttcagatttcagggnttttttttttttcctctctaagtgttccttccctttcttctcctgctctccatcatgttatggagaccagtgaggaaccagtgttaacttggtgacaatgtgacagctggtgctttatctaagctccgttttctatttcttgggaatgctttattgtggaaactgcttcagatacttaaattgaatcataacttgcttctgtaaattgcgtaaagacaacaaactgattttagtttgaaaagtttatcttttacttgtaaaccttgtttgccagttacct

SEQ ID NO: 518 - DNA, 529 bp, human
  misc_feature (60)..(64): n is a, c, g, or t
  misc_feature (68)..(70): n is a, c, g, or t
  misc_feature (74)..(83): n is a, c, g, or t
  misc_feature (86)..(87): n is a, c, g, or t
  misc_feature (89)..(90): n is a, c, g, or t
  misc_feature (92)..(92): n is a, c, g, or t
  misc_feature (95)..(95): n is a, c, g, or t
  misc_feature (99)..(99): n is a, c, g, or t
  misc_feature (101)..(103): n is a, c, g, or t
  misc_feature (111)..(112): n is a, c, g, or t
  misc_feature (115)..(115): n is a, c, g, or t
  misc_feature (121)..(121): n is a, c, g, or t
  misc_feature (124)..(124): n is a, c, g, or t
  misc_feature (127)..(127): n is a, c, g, or t
  misc_feature (129)..(129): n is a, c, g, or t
  misc_feature (432)..(432): n is a, c, g, or t
agccccaggtgtatttctgagtgtcaacagtgttaatttcaagcatgctaataaaatgtnnnnncggnnnatannnnnnnnnnaannanncnttnaaantnnnggtttccnntgntaaaancanccncntttgcttttgttagctgtaatattttttgtcatttagataagacctggtttggctctcaataaaagatgaagacagtagctctgtacagggatatatctatattagtcttcatctgatgaatgaagaaattttctcatattatgttcaagaaagtatttacttcctaaaaatagaattcccgattctgtctattttggttgaataccagaacaaatctttccgttgcaatcccagtaaaacgaaagaaaaggaatatcttacagactgttcatattagatgtatgtagactgttaatttgcantttccccatatttcctgcctatcttacccagataactttctttgaaggtaaaagctgtgcaaaaggcatgagactcaggcctactctttgtttaaat

SEQ ID NO: 519 - DNA, 281 bp, human
  misc_feature (31)..(31): n is a, c, g, or t
aatcttgtgagttgtgacctaggtgaaggtntaatgataacatctggaggaagcatcatttgttcagctacctagggtttactttcagacttttcctacagtttaaagcacttgcgttcagtgtggtacgatctgtttgtaatgttaatcattgactattgttctgctacttggatgttgatagtgaagcagagaacaatttatcatcgtttattatgagtcactatgcacgcaactatgcttaacatggcgaaatgtattaaacactcgtccaactattt

SEQ ID NO: 520 - DNA, 352 bp, human
aaagggtggcatttgcgtcacgtggaccagggacagtgctgaaatcagcagtgctcagaaacaatttaacatgttgaaacgacaatattctaaaatactgatgaatcttgcatcaatataattattgggttttttttctttttcctgctgtataactccttgccatgcaaactctcaagaggccaatatattcctggccatgtttgaatgagcctcttaaaataaacttagagccatgcaaatgccagcagcttaatggatttcatggaatgaaataccgtgattaactcatagctacatatcattgcataaatgggatttatcttttttctcacttatttttgcggtgaaa

SEQ ID NO: 521 - DNA, 245 bp, human
agagccatgcaaatgccagcagcttaatggatttcatggaatgaaataccgtgattaactcatagctacatatcattgcataaatgggatttatcttttttctcacttatttttgcggtgaaagtcgagggcatgcaagagtttctcttccagaagccaagaggagaacaaaggtcctaatgctgtactattccaccctttggacgcctcatccaggacgcagaggactctaggtttaacatttt

SEQ ID NO: 522 - DNA, 495 bp, human
gacccagagtcacctaagagaactcttccaacttaaatgactagggtgtgtaatcagttgcttcttattctgtgtctcagatttcctagtccatgacatttaatagtgagtatagagtaggatattctatacattttagtatttggcccttggaaacaaagttaaataccatgtcactcagaagactctcaaagtttgtgtataaatggcgaagtatgcactatgagagattctaatccattcacatcattcttgagttgtgcaaatacacttggctttgatttcacttggtgatctaaatttgtttatttaagctgcaagaaagttacattaatttggaatgtgtcatcacttggacccagtatatcagatttttattgagtaaaatgttaaaaataataagtgtaactcagattctggatgttcgagtttacaatacattgcctgtaataaaagctgattctgaagtgcatttttcttgacttactttggcag

SEQ ID NO: 523 - DNA, 374 bp, human
atgcacgtacaaatttcttaacctgttatcaatgtctgagctacataattatctttctagttggagtttgttttaggtgtgtaccaactgacatttcagtttttctgtttgaagtccaatgtattagtgactctgtggctgctctcttcacctgccccttgtggcctgtctacaattctaaatggattttgaactcaatgtcgtcgcttctggtttcctgcatataccaatagcattacctatgactttttttttcctgagctattttcactgagctgagctaatgaactaaaactgagttatgtttaatatttgtatcaaatacataaaaggaatactgctttttccttttgtggctcaaaggtagctgcatt

SEQ ID NO: 524 - DNA, 451 bp, human
  misc_feature (271)..(271): n is a, c, g, or t
ggaatcttttccaataaccagctaaagatttgcactgaaatacaacttgtatgccttttgcatttttaaagcctgcttcctggatttaagcagagtgatagtgttcaaagagccagttcagcctgtaacatatttgaaaaagatatgtctgcactttgaggtcccttttgaatgccattcactagacctctcaagcattttgtttcattgctacatccaagcgcctcacaagtccacaatgcgggacagcatcaaaagctcaagactttgnaaaaagcttgtgggcttgcactgggggagggaagggaacaaaatttgtgtacttctttgtttaatttagaaataaggcatccaagagatgccattattttctgtgtttcaattgttgtgcctttgagttaaactgcatttttgtcttttggttgaaatctgaaatgtactgtcccaatat

SEQ ID NO: 525 - DNA, 360 bp, human
ctccttatctgttctagttccgaagcagtttcactcgaagttgtgcagtcctggttgcagctttccgcatctgccttcgtttcgtgtagattgacgcgtttctttgtaatttcagtgtttctgacaagatttaaaaaaaaaaaaaaggaaaaaaaaagaaaaaatgaatttactgctgcaggtttttttctctctccatgtgtcactaagtgaagtttgtgccttctatagcaaagagaatattttttacatcctactaacagtagatttttttgtagtgaacattttttgtatttttatttataagtctcataagaaaaatagcaatgttcagttgtataccttgaatctgcagttaga

SEQ ID NO: 526 - DNA, 541 bp, human
aataggtcttgcaggtttccttttgaattttaagtatcataaatattttttaagtaaataatacggggtgtcagtaatatctgcagaatgaatgcagtctttcatgctaatgagttagtctggaaaaataaagtcttattttctatgttttattcatagaaatggagtattaatttttaatattttcaccatatgtgataacaaaggatctttcatgaatgtccaagggtaagtcagtattaattaatgctgtattacaaggcaatgctaccttctttattccccctttgaactacctttgaagtcactatgagcacatggatagaaatttaacttttttttgtaaagcaagcttaaaatgtttatgtatacatacccagcaacttttataaatgtgttaaacaattttactgatttttataataaatattttggtaagattttgaataatatgaattcaggcagatatactaaactgcttttatttacttgtttagaaaattgtatatatatgtttgtgtatcctaacagctgctatgaa

SEQ ID NO: 527 - DNA, 484 bp, human
  misc_feature (70)..(70): n is a, c, g, or t
gagaacatcagatctaatagagcatagtgatactatttaatttaaccaaagtctctagtgaatatttcanctttgaatgtaaactaacaaataaacctgaccaccaaggagattgtttgcccagagtttcaaagcacattgtctacaaatggaaattgaaataatttataaaatattgacgttactatgttttttaaaaagttcctaattttttcactaaatggaggaaactattagttttattgttaaatatggtagatattaatattcctcttagatgaccagtgattccaattgtcccagtttgaaataagtaccctgtgagtatgagataaattagtgacaatcagaacaagtttcagtatcagatgttcaagaggaagttgctattgcattgattttaatatttgtacataaacactgatttttttgagcattattttgtatttgttgtactttaatacctggtgtacagttccagaaa

SEQ ID NO: 528 - DNA, 399 bp, human
  misc_feature (119)..(120): n is a, c, g, or t
  misc_feature (145)..(145): n is a, c, g, or t
gaaggatgctgaggacctagatgagaagttacctgcaaaaggcaaaagggttacttagtgtcagaaccaaggcaatgacttctctctcccagatctcctagctactggtcctgggccanntttttttttttaaataatcccaacntttctttaaaagacaagcatttcagtaagctagttattttcatgggttgctcatccatttttttcagtgatctaaaaatgtagggagatggctactactgaagttgtctgtctacttgggataatagcaaattaattgaagacaatgggaaagtaagttataaaaaatactgggaaatctgtttctcttctgagcaagcattcagggcaggtataaacatcaaacatagtgacattgtcaaaacctcttccatt

SEQ ID NO: 529 - DNA, 420 bp, human
  misc_feature (78)..(78): n is a, c, g, or t
  misc_feature (161)..(161): n is a, c, g, or t
  misc_feature (163)..(165): n is a, c, g, or t
ggaaagagaaacgtggctcaagtgctggctcaccttctagctgtgtggccctgggcaggttactgaggctcttccaanctcacttttcacatgtaaaatggcatttctaaaagtacctaccctacctcaaaggaattctgaaaaaagtaagtgaatttgtnannnaagcaaagcactcacaatatgcctgacacacaaactgcttaataaatgttagtaatttttaaaacctcctcataagcggcttagaaattattcatgtttggtacatatttattgcaagtcaagcagatccaacttttttgaaagctggtggtctaggagtatagttttaattatatttgtaaatactcaaaagtattagatgccagtctaatttaaatgcagtctgactgtagcgagtaactgacctgggcaatg

SEQ ID NO: 530 - DNA, 378 bp, human
gggactcagttcttagagaagtcttttggtcttagagttcatcaagcagagaaggggaaggacatggtccaggaaaataaatcagcacacaaagagatggggtcttgagggtgcatagtacattctggacagccagttacctgggatgagttgggagggaggagaataaggacaaaagaccatctgggcaaaaatcacgaaggggtatgtgtgtcatgtaaaggtgtgccatgatagttattcatattgctattgtaatattaatatatagtaattaactacacatgacacagctttacatgaccttaagtagttatcaacattaccataatagtaatattaataactacaataagagccattattattcacttgagg

SEQ ID NO: 531 - DNA, 360 bp, human
gttagcaagtgccagcttgtaggctggttgaagtacagaactcagaggaaaaaagaaattaaattttagctttctggagagcagcccctctctggcaccatcaaacacttctttgtttcccttcaacttggaactcttcaaacatcaggggttgtgagggtttggccattcttttatcttgggtccatgtgagtgacagaaatggtgcggcctgggaaagatctccctcctttacattttctcttctccctcctcctccttattctaaaactgtgcctccaacagaggggcaggggctcttgtagagagatccctggcccaggacaggagatgccaaatctaatttatctcactgagggc

SEQ ID NO: 532 - DNA, 502 bp, human
  misc_feature (371)..(371): n is a, c, g, or t
tttcctaccaatttcacattttgcagaaacttgttcacatttccaacaatatcagaattagaaaacagttcagataacaagaaagattaaaaattagggaaattctgatatcaccataaagcactattttacatttagagattacatttaagataaagtcatcatacacaaaaacaataaatatttataactttctctataaggtccgcatatactgtatatattgaaacaatctgaatgactagtagatttcatatgaccattgttatttccactttctccaatacttgtattttatgctacatgtaatgaagttggacctttttattatttagtaattcctatatgttcctatacttttcattttcaanatgattgctctattgtttcatgttgtttctagcaatatatctccatgagatatgcactttgtttcatattgaaaagtataaaatttatctttcaattcctgtgtgtgtatcctatggttatctgtatgtat

SEQ ID NO: 533 - DNA, 258 bp, human
tcccaacccggttctggcaaagaatccagttatcccttccatgaagacgcacataactctcttacttggtctttccattagggacaacataagtcttgttttacatcaaataaaaacaatgttaaaaagtgtgtgaaccttaaaaatggaagtctactagtttacatacctacttcagaggacatggaaatgaccatgggcctgcatttcagggaccaaagcaaattaggcctggcctaaaatacatcagaccttttg

SEQ ID NO: 534 - DNA, 460 bp, human
gttgaccccaactttggaactcagatcataggatctcccagaggtttcaggtgttcctaagaagcttctaaaggaagttttggaagaaagactaataggtgtaaacatataaacatatatattatatatagctatatatatttaaagagatctctacatatatatatatataaatacaactactgtactctctgcatctgtacaaagtatgagttaaactgcccaggtagtgtgagatacgtaatgcatgaatgtgaatatcctgaaatctgccttcttagcaacgtgccgcgtgtactggagagaggacagttgctcctgggagttctctcaatgggcaaatgtacaagttgatgccgtcttatccaccaacaagccattaaggttggtttcctgctgagacttttgtaccggaagcgagaccggcagcactgggggtgtccatatgccacttatgagg

SEQ ID NO: 535 - DNA, 493 bp, human
  misc_feature (280)..(280): n is a, c, g, or t
ggccaaggcacatttgactcctgagatgaattttttgtggtcataatcaaatacttagttgtttttgatgccccaaaataaagtgagaatggtaatttgccaggaattcttcataacagtatcttacaaaaaacgtgttgctctcttcacagtattatgtgtaaagtcattgtttaaagcacgaatgttccctctggggtacttgttaaagctaaatttattttgcttccctccacttagaagtgctgcacactttacagcagcttcctttctttccatngcactgcctagttaacagaagtcttataaaaatttaaaaagacacatttcttacaaaaaagagttgaatgaggtaaaatggcattagatggctctatattttttaaagctatgtaattgttcagcgtcacttttctaagtacttatacatatctaaacatgtcttcatggtttatattttcacttatatatgctgggctggattaagctttgt

SEQ ID NO: 536 - DNA, 486 bp, human
  misc_feature (28)..(28): n is a, c, g, or t
  misc_feature (31)..(31): n is a, c, g, or t
  misc_feature (39)..(39): n is a, c, g, or t
  misc_feature (118)..(149): n is a, c, g, or t
ggggcagagggactatgttgtgagcctngcnaaagaagnttgtgtggggactgtgggcagtgaatgcgttgggaacaatatggaaaactgggagctgccttggaatctacagggccgnnnnnnnnnnnnnnnnnnnnnnnnnnnnnnnnggctgaagaaaagaagaatggtattcatgcagttcccattttacagatgaaaacaagaggactttttctgtgaagtcaagaaagtggttacaatggtactttcagcctgtccgaattatgtattgcccctcccctttttattaataacattgaagtgtgatgggacaaccactgaagccgtctgttgaaacctgctgggactttttagccattctcttcaacataaagaatgggtgtttttggagggggtgagaggaatggggaaatgttgtcaaagagtacaatgttttagttgagacaggaggaatatattttgttgagatctacagcacagcat

SEQ ID NO: 537 - DNA, 299 bp, human
gatagcgaatgcactcagggtcagcagtggagtttaaaaattgtctcttttcaacttatttaaatgacagcacctgagaagaggaaccgttttacactggatgtttctcatgtagaacaagaaatctttctggaattgatgtttacatgtctgttgttggtcatctctcctgtgtcttaaatactttaatgttggaagagcatagtgtttgggctagtgggtttctgacagcccatgggaatgccctgaaactactgtatctgatgtttgttttcgatgaggttccatgttttgttttc

SEQ ID NO: 538 - DNA, 367 bp, human
  misc_feature (27)..(27): n is a, c, g, or t
  misc_feature (32)..(32): n is a, c, g, or t
gcagctgtggtggtcattataagcatngtccntcctgggaagaagcatccaggcaagcagaaaagaaaagatgcagccaccagaaaaagaaactccagaagtcctgcatttggatgaggccaaggatcacaacagcctaaacaacctaagagaaactttgctctcagaaaagccaaacttggcccaggtggaacttgagttaaaagagagagatgtgctgtcagttttccttccggatgtaccagaaactgagagctagtgagggttcagagaagccccatcctaagccagacacatgatgtgggctcagctcagtggcctgaaacctctcaggttttagagtctctcccaagaagccgcttttttc

SEQ ID NO: 539 - DNA, 394 bp, human
  misc_feature (54)..(54): n is a, c, g, or t
  misc_feature (58)..(59): n is a, c, g, or t
  misc_feature (61)..(61): n is a, c, g, or t
  misc_feature (65)..(65): n is a, c, g, or t
  misc_feature (68)..(69): n is a, c, g, or t
  misc_feature (71)..(72): n is a, c, g, or t
  misc_feature (74)..(74): n is a, c, g, or t
  misc_feature (76)..(76): n is a, c, g, or t
  misc_feature (78)..(81): n is a, c, g, or t
  misc_feature (83)..(84): n is a, c, g, or t
  misc_feature (95)..(95): n is a, c, g, or t
  misc_feature (115)..(115): n is a, c, g, or t
  misc_feature (127)..(127): n is a, c, g, or t
  misc_feature (129)..(129): n is a, c, g, or t
  misc_feature (219)..(219): n is a, c, g, or t
tactagttgttttacactctcttttcttattcttagggcttttgtgtatgtctnactnntntttnaannanntncncnnnnanncagaccttaanttttatatttttttaaagtngctaacatagcngnaggcacttaagcatttagtcaatgatattggtagaaatagtaaaatacatcctttaaatatatatctaagcatatattttaaaaggagcnaaaataaaaccaaagtgttagtaaattttgatttattagatattttagaaaaataatagaattctgaagttttaaaaatgtcagtaattaatttattttcattttcagaaatatatgcatgcagttatgttttatttgattgttgacttaggctatgtctgtatacagtaaccaa

SEQ ID NO: 540 - DNA, 452 bp, human
  misc_feature (72)..(72): n is a, c, g, or t
  misc_feature (86)..(86): n is a, c, g, or t
  misc_feature (93)..(93): n is a, c, g, or t
  misc_feature (96)..(96): n is a, c, g, or t
  misc_feature (98)..(98): n is a, c, g, or t
  misc_feature (107)..(108): n is a, c, g, or t
  misc_feature (111)..(111): n is a, c, g, or t
  misc_feature (121)..(121): n is a, c, g, or t
  misc_feature (124)..(124): n is a, c, g, or t
  misc_feature (130)..(130): n is a, c, g, or t
  misc_feature (264)..(266): n is a, c, g, or t
  misc_feature (269)..(269): n is a, c, g, or t
  misc_feature (273)..(274): n is a, c, g, or t
  misc_feature (277)..(278): n is a, c, g, or t
  misc_feature (281)..(284): n is a, c, g, or t
  misc_feature (328)..(328): n is a, c, g, or t
  misc_feature (362)..(362): n is a, c, g, or t
  misc_feature (373)..(373): n is a, c, g, or t
  misc_feature (379)..(379): n is a, c, g, or t
gagtaggttcggtctgaaaggtgtgggcctttatatttgatccacacacgttggtcttttaaccgtgctgancagaaaacaaaacnggttaanaananccgggtggnnagntgacagaggnaanccgctnaaataccttcacaataaatagtggcaatatatatatagtttaagaaggctccccatttggcatcgtttaatttaaatgttatgttataagcacagctctcttctccaattttcatcctgcaagcaactcaaaannnttnaaannaannttnnnntgtagttattttcaaatctttgcttgataagtattaagaaatantggacttgctgccgtaatttaaagctctgttgantttgtttccgnttgganttttgggggaggggagcactgtgtttatgctggaatatgaagtctgagaccttccggtgctgggaacacacaa

SEQ ID NO: 541 - DNA, 457 bp, human
  misc_feature (364)..(366): n is a, c, g, or t
  misc_feature (369)..(369): n is a, c, g, or t
  misc_feature (379)..(379): n is a, c, g, or t
  misc_feature (382)..(383): n is a, c, g, or t
  misc_feature (398)..(398): n is a, c, g, or t
  misc_feature (407)..(407): n is a, c, g, or t
ggaacctataaatgtagtttcatctgtcgtcaattactgcagaaaattatgtgtatccacaacctagttattttaaaaattatgttgactaaatacaaagtttgttttctaaaatgtaaatatttgccacaatgtaaagcaaatcttagctatattttaaatcataaataacatgttcaagatacttaacaatttatttaaaatctaagattgctctaacgtctagtgaaaaaaatatttttaaaatttcagccaaataatgcattttatttataaaaatacagacagaaaattagggagaaacctctagttttgccaatagaaaatgcttcttccattgaataaaagttatttcaaattgaannngtncctttcacangtnnatgattaaatctganttcttaatnatatatcctatgctgattttcccaaaacatgacccatagtattaaataca

SEQ ID NO: 542 - DNA, 344 bp, human
tctttttgagtgatagtgccattgaatgagcagtatggaaacagtgttacttgatattttgagctttctcaggtttatctaaatcagtggtagcttaacaaaacccagactaattgtgtgtaattgtatttttaataaaaggaaagtacatttcctataatagcatagtactgtttgcatgtaagagtatgcaaaaccttgtgtgtgtgtgtgtgtgtgtgtgtgtgtgtgtgtcttagtgtgtgtaaggcatggcagccaactttgtatctgctatttttagtacgagcagagcttcataattgtggtcactagaactgtacttaccatggacagttaaaact

SEQ ID NO: 543 - DNA, 414 bp, human
tctctcagtttaaacatcttaacagagaatttgttactgtttattaaaagaattgcatttggaagcaagaaaacaaccggatttgcaatcatgtgaagaaacaataatgcctttattatcaagtgttaagcacaattcttataacaaactgtgataaattctttttgtttttttttcctttgccccattattttcttatgaacaaaccaaaaattcatggtgagcagttgcagtgttggctgatatatcttttatgtacagggaatttgaaaaggacagtggattcatttagaagtgtaactggtgctgtgattatagcaatacatttgttagttgtacttcatctttttcatgctagctttttaaatgtttagttttcctcttgtcatggtcagctgctgaatttacttgaag

SEQ ID NO: 544 - DNA, 545 bp, human
agattctggccacggctgggaacgttgtcaccctgaccatcatccccagtgtgatctacgagcacatggtcaaaaagttgcctccagtcctgctccaccacaccatggaccactccatcccagatgcctgaagccactgcagggcagggcaggcagggggggcttcccgccctcctgcagcaaagggcaaccaccctcggatgatgggttgcagccggcctgctgcttaaggtgggggctgccatgaggggggcgtgtccaggagggtgaccatgggatggcttatacacacaggcctccttggagcctcagactccaagctaggctgaggctcaggcagggcccacaggcagccgattctcttgtgctgatttaaatgctggacacggaggcaggctgtttaaacgctgcttaaagtcgcaactgggcccctttcaagaaattttgctctaccaggaaaacagttacacattttaagagaacagagctacgttctttgtgagagctttttccttgccttgacttgctctttgtcacagactgca

SEQ ID NO: 545 - DNA, 248 bp, human
gtcaccatctcttgttctggaagcagctccaacatcggaagtaattatgtatactggtaccagcagctcccaggaacggcccccaaactcctcatctataggaataatcagcggccctcaggggtccctgaccgattctctggctccaagtctggcacctcagcctccctggccatcagtgggctccggtccgaggatgaggctgattattactgtgcagcatgggatgacagattgagtggttttat

SEQ ID NO: 546 - DNA, 329 bp, human
  misc_feature (73)..(73): n is a, c, g, or t
  misc_feature (298)..(298): n is a, c, g, or t
gtttactctgagattctgtagtcctaatattgtatcattgtgctgtctgcaaaacaacttgaatctattttgnttgcatcttttgttacatgtaacgcagctgtactttatgttctttgcaactgtttccattatgagaacgctgtgctatttacaaggttacatttttcttggccaggcgaggtggtcatgcctgtaatcccagcactttgggaggccaaggtgggcggatcacttgaggtaaagagttgagaccagcctggctagcatggcgaaacccagtctctactaaaaatancaaaaattagccgggtgaaattagccgggcg

SEQ ID NO: 547 - DNA, 488 bp, human
ggtgttttaaggttagcacatgcacaaaggaacgagttggtttaaaaagataaatcactgcaaagaatgaaattggcttattcacatcaaaactagataagatgctaaaaaaaaaagatatgaaacagaactaacctatagtttcctgaaatcagtacagtttaatttataagaagctagaaagtaatgcaccttgattgttttaggaatgatttatgtgttgcaattttaatttatttaaagcatgtctactgtgtttgtcctaagagaaatatttcaacaaaacgtgctctgtgtttaagatatgtttaggcagtagttagcaactctgaaagtagaaactggaaatgtttattgtgaggcttgttgcagaatttccattttgtgagttactacttagtttcatgtcagcctaaaattgtaaattccctgtagatcttcaccccattgtggtgtcatcaatgaatccaaagcaggtgccattattt

SEQ ID NO: 548 - DNA, 373 bp, human
  misc_feature (67)..(67): n is a, c, g, or t
  misc_feature (74)..(74): n is a, c, g, or t
gcagggtcataatcacacagcagtgccttatatagttgccataagacttcagtgcagtacaacatanttttacngctacatatcagggcatattctatatggtgtatttgtgttagaataacacattaaatgtctttaaacataaaaataagaatgtttgcatgtttcagttttcaagaaccaaatgagtaattagctatagattccactggccttaaacatacaattaagtgtatacatgatatagtgcacacacaaaagccacctttaattattgaaataacctgtattctttttggaaatcatttaagtttggtattgaagtactatattttttgtgcatcaatgtatttttctatttacaagcctatgt

SEQ ID NO: 549 - DNA, 498 bp, human
ggagatcgtgttctgctttttacatgaggagcagaactgggccatacacgtgttcaagaactaggggagctacctggtagcaagtgagtgcagacccacctcaccttgggggaatctcaaactcataggcctcagatacacgatcacctgtcatatcaggtgagcactggcctgcttggggagagacctgggcccctccaggtgtaggaacagcaacactcctggctgacaactaagccaatatggccctaggtcattcttgcttccaatatgcttgccactccttaaatgtcctaatgatgagaaactctctttctgaccaattgctatgtttacataacacgcatgtactcatgcatcccttgccagagcccatatatgtatgcatatataaacatagcactttttactacatagctcagcacattgcaaggtttgcatttaagttaaaaaaaaaaagaaaaaaaacctaagggtgaaacgatgccactttgacgc

SEQ ID NO: 550 - DNA, 551 bp, human
  misc_feature (77)..(77): n is a, c, g, or t
  misc_feature (228)..(228): n is a, c, g, or t
  misc_feature (340)..(340): n is a, c, g, or t
  misc_feature (342)..(346): n is a, c, g, or t
  misc_feature (349)..(353): n is a, c, g, or t
  misc_feature (355)..(356): n is a, c, g, or t
  misc_feature (358)..(359): n is a, c, g, or t
  misc_feature (361)..(361): n is a, c, g, or t
tgactttcatacacacccagtacatctcaaaggatgctaagggacattttctgccagtagagttctccccctttttnggtgacagcaatattattatgttcacatctaactccagagcttacttcctgtggtgccaatgtatttgttgcaatttactacatttttatatgagcctatttataggtgccattaaactcaggtctttcaaatgaaagagtttctagcccncttagggaaaaagataattgtttagaaaaccataaaatcaatggtaggaaaagttggaactggttacctggatgccatggttctctgttaaataaagtaagagaccaggtgnannnnnaannnnnannanngntattttcagcatgctaataaatgtctttccggttatatatctatctaaaattaacctttaaaatattggtttccttgataaaagcaccactttggcttttgttagctgtaatattttgtcatttagattaagacctggttaggctctcaataaaagatgaagacagtagctctgtacagcggataatatc

SEQ ID NO: 551 - DNA, 537 bp, human
  misc_feature (109)..(109): n is a, c, g, or t
gctagttgcattatactgggtcatgaaaaattatcccttgaaatagatatgaaacatgttacttcatttctggtttaaataacttgtggaatctttcctaatgacaacntgatattaagggaaactaaagaaaatgttattgtggatcccacagtactatattacactgtttttttttgtttgttttgttagtttttttttatttaaagcaaacctcaaacattattgggtatcaattaccacctggttgtattaaaatagtaacttatcaatgccatgtaaaaattaattccattttcgaagccacctggcagacaggtttagctgtttcatcagcagcctaatatatactgttaaatttgttaaggatttcactttgaaggatacatgcaaaacatatagttactattttcatgagtcctgcttctagctccattgtggaatacagaaaattaaatatacctgttaagttcgtatctaaacctaagacattaccaaggtttgtacaaattctactacctgacatttattccaaga

SEQ ID NO: 552 - DNA, 532 bp, human
  misc_feature (69)..(69): n is a, c, g, or t
  misc_feature (131)..(131): n is a, c, g, or t
  misc_feature (283)..(283): n is a, c, g, or t
  misc_feature (412)..(412): n is a, c, g, or t
ctcccctgaagaacgtgtccagcaacgcagagtgccctgcttgttatgaatctaatggaacttcctgtngtgggaagccctggaaatgctatgaagaagaacagtgtgtctttctagttgcagaacttaanaatgacattgagtctaagagtctcgtgctgaaaggctgttccaacgtcagtaacgccacctgtcagttcctgtctggtgaaaacaagactcttggaggagtcatctttcgaaagtttgagtgtgcaaatgtaaacagcttaacccccacgtntgcaccaaccacttcccacaacgtgggctccaaagcttccctctacctcttggcccttgccagcctccttcttcggggactgctgccctgaggtcctggggctgcactttgcccagcaccccatttctncttctctgaggtccagagcaccccctgcggtgctgacaccctctttccctgctctgccccgtttaactgcccagtaagtgggagtcacaggtctccaggcaatgccgacagctgccttgt

SEQ ID NO: 553 - DNA, 521 bp, human
  misc_feature (37)..(37): n is a, c, g, or t
tttgtccatagaggcttcaagatagataggtaagagnccagtagtgttcataagaagccaatagagagcaggagccactttatcaggtggcaggtgtcctgggcctccctgctggctagtcccaagcggtggtgttgccaggatgtcttggaggtgataatgggacacacagaggcactgagtctccataggttaaaatgccaccaaaactggcctttgcctaatatccctcattgactatttagcatttaatttatttattttcctgacatttctgcaagctttgtatttatatttccactttatagatgaggaaatttgaggctcttagaggtaaaatgacttgcccaggtcacacaggaagtggcagagacaagctttttaaataagaaaaaattaataaaatataatatgagagtaacttaaaatattaataaaccacaattttaaattaattaaccgtgataaccaacattaataaaagttaagataccaaaacactggtgtctaattctttcaac

SEQ ID NO: 554 - DNA, 410 bp, human
  misc_feature (45)..(45): n is a, c, g, or t
gttctgttgtttatccatgaagcctatatatttctagtgcaaaangcccagtgggtctcaaaacattcttggaggggtttgacacactgctattagtagtgactcataattccacgattccctgggatacagagaaggcaggggggtagtactgtttgaacaaaaatcttcccaagaccctggcaaagagatcttccttaggaatcagtggcatatgcgtttatgttctttaacagggaaagtaaaagtgagcgcacttttttgaagctcataaatactcatgtgactgtaaatttagaaatgttaattaattgggcctaatgtttgatctggaaaagtacttagcagtcaccaggaatttgccttgaactgactcgaaccacaaatcagaaccgtatttccacaattta

SEQ ID NO: 555 - DNA, 296 bp, human
  misc_feature (123)..(123): n is a, c, g, or t
gcactgttaaaatccggctaggccaagatttcccaatgtcttgaagccaatggctaggaaaacagctgagcctgggactggactttgtcagtaacctctcttccctagggcacgggaggagtngggtgaataggacctcttggcttgaggaccatgattgtgaatacagacatttaattcagaatgacacctacattgatgaattttcattgtacaagtgtttttccagatcaaacacaaaacccatgtaattccttgataaattttcaatgcagggcagataaactgttgctaac

SEQ ID NO: 556 - DNA, 432 bp, human
  misc_feature (26)..(26): n is a, c, g, or t
  misc_feature (332)..(332): n is a, c, g, or t
gtgatcttggactgtcaattcccctncctgtgatccattttactgcaaacataagggttgcagtaaagggttgtctcacgtcttctgctttaaaagcctataaatatatgacctgaaaactccagttacataaaggatctgcagctatctaaggcttggttttcttactgtcatatgatacctgggtctaatgaactctgctgagatcacctcaagtttctgcggttggtaaagagaacaagggaagaacaaacatcccttttattgctccaaatggtgatttaatccctacatggtgctgggtggacaatgtgtcactgtcacatgccttnactgtataaatccaaccttctgccagagagaatctgtggttctggccatggagggaggatagtggaaatgatatagttggactggtgcttgatgtcacta

SEQ ID NO: 557 - DNA, 383 bp, human
  misc_feature (42)..(43): n is a, c, g, or t
  misc_feature (85)..(85): n is a, c, g, or t
  misc_feature (93)..(93): n is a, c, g, or t
  misc_feature (99)..(99): n is a, c, g, or t
  misc_feature (106)..(106): n is a, c, g, or t
atgtgattatacttctctttgacttgtcagcttagctttagnngatacactctggtgcccaactattattgtatcagtgaacttnccactttncttttnccttttnctctcaatttttgttgtatcattcctaccttgtgaggacatataatatttacattctgttgtcatcctcacatttcttagttccacagtttaaatgtatttgaaactcaaaacattcccattaatctcttggtcagctgaaattaatgatttaatagtttccttaaaaaagactcatggaacaatttccctaaatttttgccatgtcaaatatgtttatctgtagcctttacacagtaaaaacaatttggctagataatacaattctcagttcatat

SEQ ID NO: 558 - DNA, 391 bp, human
agggcacttctcaacattaattttggaattattcaccatttaaaaattgtcactgctctttttcattactgtagataactgagagttagctgttctaggaaaaggccatgtttcagttagtgctatgacttcttttttcgattaaggttgaagcagtgttctcttagtagcctgaacacttctcaaagcgacttatcactagcggctttaatattgagcttctatatcgttgtgtccacctcactttttaaggctgttcatacctggcaatattcacttactctgctatttttaaaaactattttaaagttttaagcacaatgttgactcttctgtaatttcctaaaattatattgttttcctacagacagctcaactttccttgatatgt

SEQ ID NO: 559 - DNA, 301 bp, human
ctcattattcctttacatgcagaatagaggcatttatgcaaattgaactgcagtttttcagcatatacacaatgtcttgtgcaacagaaaaacatgttggggaaatattcctcagtggagagtcgttctcatgctgacggggagaacgaaagtgacaggggtttcctcataagttttgtatgaaatatctctacaaacctcaattagttctactctacactttcactatcatcaacactgagactatcctgtctcacctacaaatgtggaaactttacattgttcgatttttcagcagact

SEQ ID NO: 560 - DNA, 463 bp, human
  misc_feature (226)..(226): n is a, c, g, or t
ataaatgaccagactttttctaagaaaaatgttgctttaatgcatttcatgaatttttactcttatatcattgcttgctagtaatagcaaatctgcttttctgcatctgctttgcgtagctattgtaaggctttgaactaatgtatgtatttattgcttgaacttctgtgcataccttataaagcataatgtctgacaatttaaatggctcatgtattcttgcttntatcataagctgattatggggactatgatcttttgtatacagcaaattttaaactgtagcacaaacatctgtttatgtattggtggaatatacctgttttatttatcttttttgaggtaaactaatttttgatacttttcattactgtgtactatgttcatactttgaattctctgacgttagaagtcatggttgagaattgtaacagctgttattcgttctgtattcatggctttc

SEQ ID NO: 561 - DNA, 541 bp, human
aaaagcgaggtggccatgttatgctggtggttgccagggcctccaaccactgtgccactgacttgctgtgtgaccctgggcaagtcacttaactataaggtgcctcagttttccttctgttaaaatggggataataatactgacctacctcaaagggcagttttgaggcatgactaatgctttttagaaagcattttgggatccttcagcacaggaattctcaagacctgagtattttttataataggaatgtccaccatgaacttgatacgtccgtgtgtcccagatgctgtcattagtctatatggttctccaagaaactgaatgaatccattggagaagcggtggataactagccagacaaaatttgagaatacataaacaacgcattgccacggaaacatacagaggatgccttttctgtgattgggtgggattttttccctttttatgtgggatatagtagttacttgtgacaagaataattttggaataatttctattaatatcaactctgaagctaattgtactaatctgagat

SEQ ID NO: 562 - DNA, 419 bp, human
gagtcggagatgatgcagcacacacacaattccccagcccagtgatgcttgtgttgaccagatgttcctgagtctggagcaagcacccaggccagaataacagagctttcttagttggtgaagacttaaacatctgcctgaggtcaggaggcaatttgcctgccttgtacaaaagctcaggtgaaagactgagatgaatgtctttcctctccctgcctcccaccagacttcctcctggaaaacgctttggtagatttggccaggagctttcttttatgtaaattggataaatacacacaccatacactatccacagatatagccaagtagatttgggtagaggatactatttccagaatagtgtttagctcacctagggggatatgtttgtatacacatttgcatatacccacatgggg

SEQ ID NO: 563 - DNA, 303 bp, human
ggaatgtcactggtgcccaggtgtacttgagctgtgaggtcatcggaatcccgacacctgtcctcatctggaacaaggtaaaaaggggtcactatggagttcaaaggacagaactcctgcctggtgaccgggacaacctggccattcagacccggggtggcccagaaaagcatgaagtaactggctgggtgctggtatctcctctaagtaaggaagatgctggagaatatgagtgccatgcatccaattcccaaggacaggcttcagcatcagcaaaaattacagtggttgatgccttacatg

SEQ ID NO: 564 - DNA, 192 bp, human
aggtgaaggtgccgagctataaacctccagaatattattagtctgcatggttaaaagtagtcatggataactacattacctgttcttgcctaataagtttcttttaatccaatccactaacactttagttatattcactggttttacacagagaaatacaaaataaagatcacacatcaagactatctacaa

SEQ ID NO: 565 - DNA, 449 bp, human
ctcaaagactctcccgtggatgacggatgaggactctgggctgctggaataggacactcaagacttttgactgccattttgtttgttcagtggagactccctggccaacagaatccttcttgatagtttgcaggcaaaacaaatgtaatgttgcagatccgcaggcagaagctctgcccttctgtatcctatgtatgcagtgtgctttttcttgccagcttgggccattcttgcttagacagtcagcatttgtctcctcctttaactgagtcatcatcttagtccaactaatgcagtcgatacaatgcgtagatagaagaagccccacgggagccaggatgggactggtcgtgtttgtgcttttctccaagtcagcacccaaaggtcaatgcacagagaccccgggtgggtgagcgctggcttctcaaacggccgaagttgcctctttt

SEQ ID NO: 566 - DNA, 479 bp, human
tctctctttctgtgtgtgtgtgtgtgtgtgtgtgtgtgtgtgtgtgtgtcttgtgcttcctcagacctttctcgcttctgagcttggtggcctgttccctccatctctccgaacctggcttcgcctgtccctttcactccacaccctctggccttctgccttgagctgggactgctttctgtctgtccggcctgcacccagcccctgcccacaaaaccccagggacagcggtctccccagcctgccctgctcaggccttgcccccaaacctgtactgtcccggaggaggttgggaggtggaggcccagcatcccgcgcagatgacaccatcaaccgccagagtcccagacaccggttttcctagaagcccctcacccccactggcccactggtggctaggtctccccttatccttctggtccagcgcaaggaggggctgcttctgaggtcggtggctgtctttccattaaagaaacacc

SEQ ID NO: 567 - DNA, 537 bp, human
ccaccgctggctgggaggagtcggagactgagacctacacagaggtggtgacagagtttgggaccgaggtggagcccgagtttgggaccaaggtggagcccgagtttgagacccagttggagcctgagttcgagacccagctggaacccgagtttgaggaagaggaggaggaggagaaagaggaggagatagccactggccaggcattccccttcacaacagtagagacctacacagtgaactttggggacttctgagatcagcgtcctaccaagaccccagcccaactcaagctacagcagcagcacttcccaagcctgctgaccacagtcacatcacccatcagcacatggaaggcccctggtatggacactgaaaggaagggctggtcctgcccctttgagggggtgcaaacatgactgggacctaagagccagaggctgtgtagaggctcctgctccacctgccagtctcgtaagagatggggttgctgcagtgttggagtaggggcagagggagggagccaaggtcactcca

SEQ ID NO: 568 - DNA, 180 bp, human
ctactacatgattggtgagcagaagttctccagcctccccctgggccgggaggcagtagaggctgctgtgaaagaggctggctacacaatcgaatggtttgaggtgatctcgcaaagttattcttccaccatggccaacaacgaaggacttttctccctggtggcgaggaagctgagcag

SEQ ID NO: 569 - DNA, 368 bp, human
tcgtcactgactactcagaccagaacctgcaggagctggagaagtggctgaagaaagagccagaggcctttgactggtccccagtggtgacctatgtgtgtgatcttgaagggaacagagtcaagggtccagagaaggaggagaagttgagacaggcggtcaagcaggtgctgaagtgtgatgtgactcagagccagccactgggggccgtccccttacccccggctgactgcgtgctcagcacactgtgtctggatgccgcctgcccagacctccccacctactgcagggcgctcaggaacctcggcagcctactgaagccagggggcttcctggtgatcatggatgcgctcaagagcagctactac

SEQ ID NO: 570 - DNA, 473 bp, human
tggcctacatggaccagcagactggcaacctcaagaaggccctgctcctccagggctccaacgagatcgagatccgcgccgagggcaacagccgcttcacctacagcgtcactgtcgatggctgcacgagtcacaccggagcctggggcaagacagtgattgaatacaaaaccaccaagacctcccgcctgcccatcatcgatgtggcccccttggacgttggtgccccagaccaggaattcggcttcgacgttggccctgtctgcttcctgtaaactccctccatcccaacctggctccctcccacccaaccaactttccccccaacccggaaacagacaagcaacccaaactgaaccccctcaaaagccaaaaaatgggagacaatttcacatggactttggaaaatatttttttcctttgcattcatctctcaaacttagtttttatctttgaccaaccgaacatgacca

SEQ ID NO: 571 - DNA, 472 bp, human
  misc_feature (149)..(149): n is a, c, g, or t
  misc_feature (153)..(153): n is a, c, g, or t
  misc_feature (428)..(428): n is a, c, g, or t
gctccccatttttataccaaaggtgctacatctatgtgatgggtggggtggggagggaatcactggtgctatagaaattgagatgcccccccaggccagcaaatgttcctttttgttcaaagtctatttttattccttgatattttttntttntttttttttttttttgtggatggggacttgtgaatttttctaaaggtgctatttaacatgggaggagagcgtgtgcggctccagcccagcccgctgctcactttccaccctctctccacctgcctctggcttctcaggcctctgctctccgacctctctcctctgaaaccctcctccacagctgcagcccatcctcccggctccctcctagtctgtcctgcgtcctctgtccccgggtttcagagacaacttcccaaagcacaaagcagtttttncccctaggggtgggaggaagcaaaagactctgtacctattttgt

SEQ ID NO: 572 - DNA, 356 bp, human
actttcccatgagtgtgatccacattgttaggtgctgacctagacagagatgaactgaggtccttgttttgttttgttcataatacaaaggtgctaattaatagtatttcagatacttgaagaatgttgatggtgctagaagaatttgagaagaaatactcctgtattgagttgtatcgtgtggtgtattttttaaaaaatttgatttagcattcatattttccatcttattcccaattaaaagtatgcagattatttgcccaaagttgtcctcttcttcagattcagcatttgttctttgccagtctcattttcatcttcttccatggttccacagaagctttgtttcttgggca

SEQ ID NO: 573 - DNA, 503 bp, human
gaacaacgcctgtggcattgccaacctggccagcttccccaagatgtgactccagccagccaaatccatcctgctcttccatttcttccacgatggtgcagtgtaacgatgcactttggaagggagttggtgtgctatttttgaagcagatgtggtgatactgagattgtctgttcagtttccccatttgtttgtgcttcaaatgatccttcctactttgcttctctccacccatgacctttttcactgtggccatcaggactttccctgacagctgtgtactcttaggctaagagatgtgactacagcctgcccctgactgtgttgtcccagggctgatgctgtacaggtacaggctggagattttcacataggttagattctcattcacgggactagttagctttaagcaccctagaggactagggtaatctgacttctcacttcctaagttcccttctatatcctcaaggtagaaatgtctatgttttctactccaattc

SEQ ID NO: 574 - DNA, 364 bp, human
gtacccagttaaattttcatttcagataaacaacaaataattttttagtataagtacattattgtttatctgaaagttttaattgaactaacaatcctagtttgatactcccagtcttgtcattgccagctgtgttggtagtgctgtgttgaattacggaataatgagttagaactattaaaacagccaaaactccacagtcaatattagtaatttcttgctggttgaaacttgtttattatgtacaaatagattcttataatattatttaaatgactgcatttttaaatacaaggctttatatttttaactttaagatgtttttatgtgctctccaaattttttttactgtttctgattgtat

SEQ ID NO: 575 - DNA, 557 bp, human
atccgaattctccatatattcactaatcaaagacactattttcatactagattcctgagacaaatactcactgaagggcttgtttaaaaataaattgtgttttggtctgttcttgtagataatgcccttctattttaggtagaagctctggaatccctttattgtgctgttgctcttatctgcaaggtggcaagcagttcttttcagcagattttgcccactattcctctgagctgaagttctttgcatagatttggcttaagcttgaattagatccctgcaaaggcttgctctgtgatgtcagatgtaattgtaaatgtcagtaatcacttcatgaatgctaaatgagaatgtaagtatttttaaatgtgtgtatttcaaatttgtttgactaattctggaattacaagatttctatgcaggatttaccttcatcctgtgcatgtttcccaaactgtgaggagggaaggctcagagatcgagcttctcctctgagttctaacaaaatggtgctttgagggtcagcctttaggaaggtgcagctttgttgtcctttg

SEQ ID NO: 576 - DNA, 168 bp, human
ggcatcatgttgaccgagctggagaaagccttgaactctatcatcgacgtctaccacaagtactccctgataaaggggaatttccatgccgtctacagggatgacctgaagaaattgctagagaccgagtgtcctcagtatatcaggaaaaagggtgcagacgtctgg

SEQ ID NO: 577 - DNA, 557 bp, human
gccagtcttgaccgggatgaggcccacagacaggttgtcatcagcttgtcccattcaagccaccgagctcaccacagacacagtggagccgcgctcttctccagtgacacgtggacaaatgcgggctcatcagcccccccagagagggtcaggccgaaccccatttctcctcctcttaggtcattttcagcaaacttgaatatctagacctctcttccaatgaaaccctccagtctattatagtcacatagataatggtgccacgtgttttctgatttggtgagctcagacttggtgcttccctctccacaacccccaccccttgtttttcaagatactattattatattttcacagacttttgaagcacaaatttattggcatttaatattggacatctgggcccttggaagtacaaatctaaggaaaaaccaacccactgtgtaagtgactcatcttcctgttgttccaattctgtgggtttttgattcaacggtgctataaccagggtcctgggtgacagggcgctcactgagcaccatgtgtcatcacagaca

SEQ ID NO: 578 - DNA, 546 bp, human
caggaaatagtcactcatcccactccacataaggggtttagtaagagaagtctgtctgtctgatgatggatagggggcaaatctttttcccctttctgttaatagtcatcacatttctatgccaaacaggaacgatccataactttagtcttaatgtacacattgcattttgataaaattaattttgttgtttcctttgaggttgatcgttgtgttgttttgctgcactttttacttttttgcgtgtggagctgtattcccgagacaacgaagcgttgggatacttcattaaatgtagcgactgtcaacagcgtgcaggttttctgtttctgtgttgtggggtcaaccgtacaatggtgtgggaatgacgatgatgtgaatatttagaatgtaccatattttttgtaaattatttatgtttttctaaacaaatttatcgtataggttgatgaaacgtcatgtgttttgccaaagactgtaaatatttatttatgtgttcacatggtcaaaatttcaccactgaaaccctgcacttagctagaacct

SEQ ID NO: 579 - DNA, 361 bp, human
  misc_feature (75)..(75): n is a, c, g, or t
cccacctggagctgaatcacatgacctagctgcaccccagcgcctgggcccgccccacgctctgtccacacccangcgccccgggagcggggccatgcctccagccccccagctcgcccgacccatcctgttcgtgaataggtctcaggggttgggggagggactgccagatttggacactatatttttttctaaattcaacttgaagatgtgtatttcccctgaccttcaaaaaatgttccaaggtaagcctcgtaaaggtcatcccaccatcaccaaagcctccgtttttaacaacctccaacacgatccatttagaggccaaatgtcattctgcaggtgccttcccgatggattaaag

SEQ ID NO: 580 - DNA, 549 bp, human
ctggaggacagcacctgtgacttcggcaacctcaagcgctatgcatgcacctctcatacccagggcctgagcccaggctgctatgacacctacaatgcggacatcgactgccagtggatcgacataaccgacgtgcagcctgggaactacatcctcaaggtgcacgtgaacccaaagtatattgttttggagtctgacttcaccaacaacgtggtgagatgcaacattcactacacaggtcgctacgtttctgcaacaaactgcaaaattgtccaatcctgatctccgggagggacagatggccaatctctccccttccaaagcaggccctgctccccgggcagcctcccgccgaggggcccagcccccaacccacaggcagggaggggcatccctccctgccggcctcagggagcgaacgtggatgaaaaccacagggattccggatgccagaccccattttatacttcacttttctctacagtgttgttttgttgttgttggtttttattttttatactttggccataccacagagctagattgccc

SEQ ID NO: 581 - DNA, 539 bp, human
tgccagcgactgtctcagactgggcagggaggctttggcatgacttaagaggaagggcagtcttgggacccgctatgcaggtcctggcaaacctggctgccctgtctcatccctgtccctcagggtagcaccatggcaggactgggggaactggagtgtccttgctgtatccctgttgtgaggttccttccaggggctggcactgaagcaagggtgctggggccccatggccttcagccctggctgagcaactgggctgtagggcagggccacttcctgaggtcaggtcttggtaggtgcctgcatctgtctgccttctggctgacaatcctggaaatctgttctccagaatccaggccaaaaagttcacagtcaaatggggaggggtattcttcatgcaggagaccccaggccctggaggctgcaacatacctcaatcctgtcccaggccggatcctcctgaagcccttttcgcagcactgctatcctccaaagccattgtaaatgtgtgtacagtgtgtataaaccttcttcttctt

SEQ ID NO: 582 - DNA, 512 bp, human
gagtacaggtgccagacaaacctctccaccctcagtgacccggtgcagctagaagtccatatcggctggctgttgctccaggcccctcggtgggtgttcaaggaggaagaccctattcacctgaggtgtcacagctggaagaacactgctctgcataaggtcacatatttacagaatggcaaagacaggaagtattttcatcataattctgacttccacattccaaaagccacactcaaagatagcggctcctacttctgcagggggcttgttgggagtaaaaatgtgtcttcagagactgtgaacatcaccatcactcaaggtttggcagtgtcaaccatctcatcattctctccacctgggtaccaagtctctttctgcttggtgatggtactcctttttgcagtggacacaggactatatttctctgtgaagacaaacatttgaagctcaacaagagactggaaggaccataaacttaaatggagaaaggaccctcaagacaaatgacc

SEQ ID NO: 583 - DNA, 291 bp, human
aaccagccagtcccaagaagaacattaaaactaggagtgcccagaagagaacaaacccgaaaagagtgtgagctaactagtttccaaagcggagacttccgacttccttacaggatgaggctgggcattgcctgggacagcctatgtaaggccatgtgccccttgccctaacaactcactgcagtgctcttcatagacacatcttgcagcatttttcttaaggctatgcttcagtttttctttgtaagccatcacaagccatagtggtaggtttgccctttggtacagaag

SEQ ID NO: 584 - DNA, 507 bp, human
gaaaatgtaccttggtgccaccaacccattttgtgccacatgcaagttttgaataaggatgtatggaaaacaacgctgcatatacaggtaccatttaggaaataccgatgcctttgtgggggcagaatcacagacaaaagctttgaaaatcataaagatataagttggtgtggctaagatggaaacagggctgattcttgattcccaattctcaactctccttttcctatttgaatttctttggtgctgtagaaaacaaaaaaagaaaaatatatattcataaaaaatatggtgctcattctcatccatccaggatgtactaaaacagtgtgtttaataaattgtaattattttgtgtacagttctatactgttatctgtgtccatttccaaaacttgcacgtgtccctgaattccgctgactctaatttatgaggatgccgaactctgatggcaataatatatgtattatgaaaatgaagttatgatttccgatgaccctaagtcc

SEQ ID NO: 585 - DNA, 386 bp, human
gaagaactgtctattttctcagtcatttttaacctctagagtcactgatacacagaatataatcttatttatacctcagtttgcatatttttttactatttagaatgtagccctttttgtactgatataatttagttccacaaatggtgggtacaaaaagtcaagtttgtggcttatggattcatataggccagagttgcaaagatcttttccagagtatgcaactctgacgttgatcccagagagcagcttcagtgacaaacatatcctttcaagacagaaagagacaggagacatgagtctttgccggaggaaaagcagctcaagaacacatgtgcagtcactggtgtcaccctggataggcaagggataactcttctaacaca

SEQ ID NO: 586 - DNA, 541 bp, human
tgccgtgctcccaaaacattttaaatgaaagtattggcattcaaaaagacagcagacaaaatgaaagaaaatgagagcagaaagtaagcatttccagcctatctaatttctttagttttctatttgcctccagtgcagtccatttcctaatgtataccagcctactgtactatttaaaatgctcaatttcagcaccgatggccatgtaaataagatgatttaatgttgattttaatcctgtatataaaataaaaagtcacaatgagtttgggcatatttaatgatgattatggagccttagaggtctttaatcattggttcggctgcttttatgtagtttaggctggaaatggtttcacttgctctttgactgtcagcaagactgaagatggcttttcctggacagctagaaaacacaaaatcttgtaggtcattgcacctatctcagccataggtgcagtttgcttctacatgatgctaaaggctgcgaatgggatcctgatggaactaaggactccaatgtcgaactcttctttgctgc

SEQ ID NO: 587 - DNA, 524 bp, human
ggggaactgagggcaaggggatatagtgatggggctcagatggactgggaggagggggagggtgatgcattaattaatggcttcgttaattaatgtcatgttgcttgtcgctttctcagtgtgtgtgtgtggtccatgcccactgctggtgccagggtgggtgtccatgtgcacccggcctggatgccagctgtgtccttcgggggcgtgcgtgtaactgtagtgtagtcaggtgctcaatggagaatataaacatatacagaaaaatatatattttaagtttaaaaaacagaaaaacagacaaaacaatccccatcaggtagctgtctaacccccagctgggtctaatccttctcattacccacccgacctggctgcccctcaccttgggctgggggactggggggccatttccttttctctgccctttttttgttgttctattttgtacagacaagttggaaaaacaacagcgacaaaaaagtcaagaaactttgtaaaatatcgtgtgtgtgattccttgt

SEQ ID NO: 588 - DNA, 502 bp, human
cccgaccggtgggcatttgtgaggcccatggttgagaaatgaataatttcccaattaggaagtgtaagcagctgaggtctcttgagggagcttagccaatgtgggagcagcggtttggggagcagagacactaacgacttcagggcagggctctgatattccatgaatgtatcaggaaatatatatgtgtgtgtatgtttgcacacttgttgtgtgggctgtgagtgtaagtgtgagtaagagctggtgtctgattgttaagtctaaatatttccttaaactgtgtggactgtgatgccacacagagtggtctttctggagaggttataggtcactcctggggcctcttgggtcccccacgtgacagtgcctgggaatgtacttattctgcagcatgacctgtgaccagcactgtctcagtttcactttcacatagatgtccctttcttggccagttatcccttccttttagcctagttcatccaatcctcactgggtgggg

SEQ ID NO: 589 - DNA, 555 bp, human
caccacctacctatgatgccgtggtacagatggagtaccttgacatggtggtgaatgaaacactcagattattcccagttgctattagacttgagaggacttgcaagaaagatgttgaaatcaatggggtattcattcccaaagggtcaatggtggtgattccaacttatgctcttcaccatgacccaaagtactggacagagcctgaggagttccgccctgaaaggttcagtaagaagaaggacagcatagatccttacatatacacaccctttggaactggacccagaaactgcattggcatgaggtttgctctcatgaacatgaaacttgctctaatcagagtccttcagaacttctccttcaaaccttgtaaagaaacacagatccccttgaaattagacacgcaaggacttcttcaaccagaaaaacccattgttctaaaggtggattcaagagatggaaccctaagtggagaatgagttattctaaggacttctactttggtcttcaagaaagctgtgccccagaacaccagagatttcaacttagtca

SEQ ID NO: 590 - DNA, 540 bp, human
gaaaatcgatgcagccatttctgataaggaaaagaacaaaacatatttctttgtagaggacaaatactggagatttgatgagaagagaaattccatggagccaggctttcccaagcaaatagctgaagactttccagggattgactcaaagattgatgctgtttttgaagaatttgggttcttttatttctttactggatcttcacagttggagtttgacccaaatgcaaagaaagtgacacacactttgaagagtaacagctggcttaattgttgaaagagatatgtagaaggcacaatatgggcactttaaatgaagctaataattcttcacctaagtctctgtgaattgaaatgttcgttttctcctgcctgtgctgtgactcgagtcacactcaagggaacttgagcgtgaatctgtatcttgccggtcatttttatgttattacagggcattcaaatgggctgctgcttagcttgcaccttgtcacatagagtgatctttcccaagagaaggggaagcactcgtgtgcaacagac

SEQ ID NO: 591 - DNA, 511 bp, human
tccacacacggccaggcctgtttatctacactgctgcccactcctctctccagctccacatgctgtacctggatcattctgaagcaaattccgagcattacatcattttgtccataaatatttctaacatccttaaatatacaatcggaattcaagcatctcccattgtcccacaaatgtttggctgtttttgtagttggattgtttgtattaggattcaagcaaggcccatatattgcatttatttgaaatgtctgtaagtctctttccatctacagagtttagcacatttgaacgttgctggttgaaatcccgaggtgtcatttgacatggttctctgaacttatctttcctataaaatggtagttagatctggaggtctgattttgtggcaaaaatacttcctaggtggtgctgggtacttcttgttgcatcctgtcaggaggcagataatgctggtgcctctctattggtaatgttaagactgctgggtgggtttggagttcttggc

SEQ ID NO: 592 - DNA, 428 bp, human
gaacatccaagtctttcttcttttttaagttgtcaaagaagcttccacaaaattagaaaggacaacagttctgagctgtaatttcgccttaaactctggacactctatatgtagtgcatttttaaacttgaaatatataatattcagccagcttaaacccatacaatgtatgtacaatacaatgtacaattatgtctcttgagcatcaatcttgttactgctgattcttgtaaatctttttgcttctactttcatcttaaactaatacgtgccagatataactgtcttgtttcagtgagagacgccctatttctatgtcatttttaatgtatctatttgtacaattttaaagttcttattttagtatacatataaatatcagtattctgacatgtaagaaaatgttacggcatcacacttatatttta

SEQ ID NO: 593 - DNA, 541 bp, human
aatctcttctagagcacctgctatccccaacttctagacctgctccaaactagtgactaggatagaatttgatcccctaactcactgtctgcggtgctcattgctgctaacagcattgcctgtgctctcctctcaggggcagcatgctaacggggcgacgtcctaatccaactgggagaagcctcagtggtggaattccaggcactgtgactgtcaagctggcaagggccaggattgggggaatggagctggggcttagctgggaggtggtctgaagcagacagggaatgggagaggaggatgggaagtagacagtggctggtatggctctgaggctccctggggcctgctcaagctcctcctgctccttgctgttttctgatgatttgggggcttgggagtccctttgtcctcatctgagactgaaatgtggggatccaggatggcttccttcctcttacccttcctccctcagcctgcaacctctatcctggaacctgtcctccctttctccccaactatgcatctgttgtctgctcct

SEQ ID NO: 594 - DNA, 502 bp, human
aagaagctaagcctgatctcctgtaaaaacatcatttccctgatggacaccagcggcaatgggaagctggagtttgatgaattcaaagtgttctgggacaagctgaagcagtggattaaccttttccttcggtttgatgctgacaagtccggcaccatgtctacctatgaactacggactgcactgaaagctgcaggctttcagctgagcagccacctcctgcagctgattgtgctcaggtatgcggatgaggagctccagctggacttcgatgacttcctcaactgcctggtccggctggagaatgcgagccgggtgttccaggctctcagtacaaagaacaaggagttcattcatctcaatataaatgagttcatccatttgacaatgaacatctgaggctgccttgtagagatgcagcctgcccagctgaatcttggcttctggaccttgaccttcagaacttctcttggtgtggaaccattacgcccagggttcactc

SEQ ID NO: 595 - DNA, 542 bp, human
actcccaagagcaaatccacattcctcttgagttctgcagcttctgtgtaaatagggcagctgtcgtctatgccgtagaatcacatgatctgaggaccattcatggaagctgctaaatagcctagtctggggagtcttccataaagttttgcatggagcaaacaaacaggattaaactaggtttggttccttcagccctctaaaagcatagggcttagcctgcaggcttccttgggctttctctgtgtgtgtagttttgtaaacactatagcatctgttaagatccagtgtccatggaaacattcccacatgccgtgactctggactatatcagtttttggaaagcagggttcctctgcctgctaacaagcccacgtggaccagtctgaatgtctttcctttacacctatgtttttaagtagtcaaacttcaagaaacaatctaaacaagtttctgttgcatatgtgtttgtgaacttgtatttgtatttagtaggcttctatattgcatttaacttgtttttgtaactcctgattcttcct

SEQ ID NO: 596 - DNA, 473 bp, human
gaagcctcaagttccagtgcagagatcctacttctctgagtcagctgaccccctccccgcaatccctcaaaccttgaggagaagtggggaccccacccctcatcaggagttccagtgctgcatgcgattatctacccacgtccacgcggccacctcaccctctccgcacacctctggctgtctttttgtactttttgttccagagctgcttctgtctggtttatttaggttttatccttccttttctttgagagttcgtgaagagggaagccaggattggggacctgatggagagtgagagcatgtgaggggtagtgggatggtggggtaccagccactggaggggtcatccttgcccatcgggaccagaaacctgggagagacttggatgaggagtggttgggctgtgcctgggcctagcacggacatggtctgtcctgacagcactcctcggcaggcatggctggtgcctg

SEQ ID NO: 597 - DNA, 477 bp, human
aaagcagcgctagtggagggttggagaaggaggtaaggatgagggttcatcatccctccctgcctaaggaagctaaaagcatggccctgctgcccctccctgcctccacccacagtggagagggctacaaaggaggacaagaccctctcaggctgtcccaagctcccaagagcttccagagctctgacccacagcctccaagtcaggtggggtggagtcccagagctgcacagggtttggcccaagtttctaagggaggcacttcctcccctcgcccatcagtgccagcccctgctggctggtgcctgagcccctcagacagccccctgccccgcaggcctgccttctcagggacttctgcggggcctgaggcaagccatggagtgagacccaggagccggacacttctcaggaaatggcttttcccaacccccagcccccacccggtggttcttcctgttctgtgactgtgtatag

SEQ ID NO: 598 - DNA, 432 bp, human
ttcagccagtcgccaagaatcatgaaagtcgccagtggcagcaccgccaccgccgccgcgggccccacgtgcgcgctgaaggccggcaagacagcgagcggtgcgggcgaggtggtgcgctgtctgtctgagcagagcgtggccatctcgcgctgccggggcgccggggcgcgcctgcctgccctgctggacgagcagcaggtaaacgtgctgctctacgacatgaacggctgttactcacgcctcaaggagctggtgcccaccctgccccagaaccgcaaggtgagcaaggtggagattctccagcacgtcatcgactacatcagggaccttcagttggagctgaactcggaatccgaagttggaacccccgggggccgagggctgccggtccgggctccgctcagcaccctcaacggcgagatcagcgcc

SEQ ID NO: 599 - DNA, 382 bp, human
gattccctgcatcaactaagaaaagcctgttttctttatttcaaacttggtggcgaatgtgttgcgggtcctgttgggctgctttctgtattgtctcctaaccctctagttttaattggacacttctttgctgttgcaatctatgccgtgtatttttgctttaagtcagaaccttggattacaaaacctcgagcccttctcagtagtggtgctgtattgtacaaagcgtgttctgtaatatttcctctaatttactcagaaatgaagtatatggttcattaagcttaaaggggaaccatttgtgaatgaatatttggaacttaccaagtcctaagagacttttggaagaggatatatatagcatagtaccataccacttata

SEQ ID NO: 600 - DNA, 269 bp, human
gctgtggggatagtgaggcatcgcaatgtaagactcgggattagtacacacttgttgatgattaatggaaatgtttacagatccccaagcctggcaagggaatttcttcaactccctgccccctagccctccttatcaaaggacaccattttggcaagctctatcaccaaggagccaaacatcctacaagacacagtgaccatactaattataccccctgcaaagccagcttgaaaccttcacttaggaacgtaatcgtgtcccctatc

SEQ ID NO: 601 - DNA, 473 bp, human
tcaccaatgccatcaaggatgcactcgctgcaacgtagccctctgttctgcacacagcacgggggccaaggatgccccgtccccctctggctccagctggccgggagcctgatcacctgccctgctgagtcccaggctgagcctcagtctccctcccttggggcctatgcagaggtccacaacacacagatttgagctcagccctggtgggcagagaggtagggatggggctgtggggatagtgaggcatcgcaatgtaagactcgggattagtacacacttgttgatgattaatggaaatgtttacagatccccaagcctggcaagggaatttcttcaactccctgccccctagccctccttatcaaaggacaccattttggcaagctctatcaccaaggagccaaacatcctacaagacacagtgaccatactaattataccccctgcaaagccagcttgaaaccttcact

SEQ ID NO: 602 - DNA, 329 bp, human
tccaacctccttcaaattcagtcaccactgttatattaccttctccaggaaccctccagtggggaaggctgcgatattagatttccttgtatgcaaagtttttgttgaaagctgtgctcagaggaggtgagaggagaggaaggagaaaactgcatcataactttacagaattgaatctagagtcttccccgaaaagcccagaaacttctctgcagtatctggcttgtccatctggtctaaggtggctgcttcttccccagccatgagtcagtttgtgcccatgaataatacacgacctgttatttccatgactgctttactgtattttt

SEQ ID NO: 603 - DNA, 529 bp, human
gaatggtgcatacaaggccatccccgttgcccaggacctgaacgcgccttctgattgggacagccgtgggaaggacagttatgaaacgagtcagctggatgaccagagtgctgaaacccacagccacaagcagtccagattatataagcggaaagctaatgatgagagcaatgagcattccgatgtgattgatagtcaggaactttccaaagtcagccgtgaattccacagccatgaatttcacagccatgaagatatgctggttgtagaccccaaaagtaaggaagaagataaacacctgaaatttcgtatttctcatgaattagatagtgcatcttctgaggtcaattaaaaggagaaaaaatacaatttctcactttgcatttagtcaaaagaaaaaatgctttatagcaaaatgaaagagaacatgaaatgcttctttctcagtttattggttgaatgtgtatctatttgagtctggaaataactgatgtgtttgataattagtttagtttgtggcttcatggaa

SEQ ID NO: 604 - DNA, 536 bp, human
acagctttccaaggtgacaaactcctctatgcagtgtatcgaaagctgggtgtttatgaagttgaagaccagattacagctgtcagaaaattcatagaaatgggtttcattgatgaaaaaagaatagccatatggggctggtcctatggaggatacgtttcatcactggcccttgcatctggaactggtcttttcaaatgtggtatagcagtggctccagtctccagctgggaatattacgcgtctgtctacacagagagattcatgggtctcccaacaaaggatgataatcttgagcactataagaattcaactgtgatggcaagagcagaatatttcagaaatgtagactatcttctcatccacggaacagcagatgataatgtgcactttcagaactcagcacagattgctaaagctctggttaatgcacaagtggatttccaggcaatgtggtactctgaccagaaccacggcttatccggcctgtccacgaaccacttatacacccacatgacccacttcctaaagcagtg

SEQ ID NO: 605 - DNA, 470 bp, human
tcttttgtcctccttagcacaatgtaaaaaagaatagtaatatcagaacaggaaggaggaatggcttgctggggagcccatccaggacactgggagcacatagagattcacccatgtttgttgaacttagagtcattctcatgcttttctttataattcacacatatatgcagagaagatatgttcttgttaacattgtatacaacatagccccaaatatagtaagatctatactagataatcctagatgaaatgttagagatgctatatgatacaactgtggccatgactgaggaaaggagctcacgcccagagactgggctgctctcccggaggccaaacccaagaaggtctggcaaagtcaggctcagggagactctgccctgctgcagacctcggtgtggacacacgctgcatagagctctccttgaaaacagaggggtctcaagacattctgcctacctattagc

SEQ ID NO: 606 - DNA, 441 bp, human
aaaggagcagtcgcacagacctttcctcatgctgcaggcccggcagtctgaagaccaccctcatcgccggcgtcggcggggcttggagtgtgatggcaaggtcaacatctgctgtaagaaacagttctttgtcagtttcaaggacatcggctggaatgactggatcattgctccctctggctatcatgccaactactgcgagggtgagtgcccgagccatatagcaggcacgtccgggtcctcactgtccttccactcaacagtcatcaaccactaccgcatgcggggccatagcccctttgccaacctcaaatcgtgctgtgtgcccaccaagctgagacccatgtccatgttgtactatgatgatggtcaaaacatcatcaaaaaggacattcagaacatgatcgtggaggagtgtgggtgctcatagagttgcccagc

SEQ ID NO: 607 - DNA, 561 bp, human
acaagcatcctgtctcacgaagaacaaatgtttgttaatcgtgtgggccatgattatcagtggataggcctcaatgacaagatgtttgagcatgacttccgttggactgatggcagcacactgcaatacgagaattggagacccaaccagccagacagcttcttttctgctggagaagactgtgttgtaatcatttggcatgagaatggccagtggaatgatgttccctgcaattaccatctcacctatacgtgcaagaaaggaacagttgcttgcggccagccccctgttgtagaaaatgccaagacctttggaaagatgaaacctcgttatgaaatcaactccctgattagataccactgcaaagatggtttcattcaacgtcaccttccaactatccggtgcttaggaaatggaagatgggctatacctaaaattacctgcatgaacccatctgcataccaaaggacttattctatgaaatactttaaaaattcctcatcagcaaaggacaattcaataaatacatccaaacatgatcatcgttggagccggaggtgg

SEQ ID NO: 608 - DNA, 557 bp, human
tggtgatgtctgctactatgccagccggaacgacaagtcctactggctctctaccactgcgccgctgcccatgatgcccgtggccgaggacgagatcaagccctacatcagccgctgttctgtgtgtgaggccccggccatcgccatcgcggtccacagtcaggatgtctccatcccacactgcccagctgggtggcggagtttgtggatcggatattccttcctcatgcacacggcggcgggagacgaaggcggtggccaatcactggtgtcaccgggcagctgtctagaggacttccgcgccacaccattcatcgaatgcaatggaggccgcggcacctgccactactacgccaacaagtacagcttctggctgaccaccattcccgagcagagcttccagggctcgccctccgccgacacgctcaaggccggcctcatccgcacacacatcagccgctgccaggtgtgcatgaagaacctgtgagccggcgcgtgccaggaagggccattttggtgcttattcttaacttattacctcaggtgccaacccaaaa

SEQ ID NO: 609 - DNA, 436 bp, human
gaaagactgtgctgtcctttaacataggtttttaaagactaggatattgaatgtgaaacatccgttttcattgttcacttctaaaccaaaaattatgtgttgccaaaaccaaacccaggttcatgaatatggtgtctattatagtgaaacatgtactttgagcttattgtttttattctgtattaaatattttcagggttttaaacactaatcacaaactgaatgacttgacttcaaaagcaacaaccttaaaggccgtcatttcattagtattcctcattctgcatcctggcttgaaaaacagctctgttgaatcacagtatcagtattttcacacgtaagcacattcgggccatttccgtggtttctcatgagctgtgttcacagacctcagcagggcatcgcatggaccgcaggagggcagattcggaccact

SEQ ID NO: 610 - DNA, 429 bp, human
tcggctactcttttgtgatgcacaccagcgctggtgcagaaggctctggccaagccctggcgtcccccggctcctgcctggaggagtttagaagtgcgccattcatcgagtgtcacggccgtgggacctgcaattactacgcaaacgcttacagcttttggctcgccaccatagagaggagcgagatgttcaagaagcctacgccgtccaccttgaaggcaggggagctgcgcacgcacgtcagccgctgccaagtctgtatgagaagaacataatgaagcctgactcagctaatgtcacaacatggtgctacttcttcttctttttgttaacagcaacgaaccctagaaatatatcctgtgtacctcactgtccaatatgaaaaccgtaaagtgccttataggaatttgcgtaactaacacaccctgc

SEQ ID NO: 611 - DNA, 517 bp, human
  misc_feature (181)..(181): n is a, c, g, or t
ggaaaacacctcatttgaccttgccagctgaccttcaaaccctgcatttgaaccgaccaacattaagtccagagagtaaacttgaatggaataacgacattccagaagttaatcatttgaattctgaacactggagaaaaaccgaaaaatggacggggcatgaagagactaatcatctggnaaaccgatttcagtggcgatggcatgacagagctagagctcgggcccagccccaggctgcagcccattcgcaggcacccgaaagaacttccccagtatggtggtcctggaaaggacatttttgaagatcaactatatcttcctgtgcattccgatggaatttcagttcatcagatgttcaccatggccaccgcagaacaccgaagtaattccagcatagcggggaagatgttgaccaaggtggagaagaatcacgaaaaggagaagtcacagcacctagaaggcagcgcctcctcttcactctcctctgattagatgaaactgttaccttacccta

SEQ ID NO: 612 - DNA, 397 bp, human
aatatccttgttgtgtattaggtttttaaataccagctaaaggattacctcactgagtcatcagtaccctcctattcagctccccaagatgatgtgtttttgcttaccctaagagaggttttcttcttatttttagataattcaagtgcttagataaattatgttttctttaagtgtttatggtaaactcttttaaagaaaatttaatatgttatagctgaatctttttggtaactttaaatctttatcatagactctgtacatatgttcaaattagctgcttgcctgatgtgtgtatcatcggtgggatgacagaacaaacatatttatgatcatgaataatgtgctttgtaaaaagatttcaagttattaggaagcatactctgttttttaatca

SEQ ID NO: 613 - DNA, 518 bp, human
gtgtgcacacggagactcatcgttataatttactatctgccaagagtagaaagaaaggctggggatatttgggttggcttggttttgattttttgcttgtttgtttgttttgtactaaaacagtattatcttttgaatatcgtagggacataagtatatacatgttatccaatcaagatggctagaatggtgcctttctgagtgtctaaaacttgacacccctggtaaatctttcaacacacttccactgcctgcgtaatgaagttttgattcatttttaaccactggaatttttcaatgccgtcattttcagttagatgattttgcactttgagattaaaatgccatgtctatttgattagtcttatttttttatttttacaggcttatcagtctcactgttggctgtcattgtgacaaagtcaaataaacccccaaggacgacacacagtatggatcacatattgtttgacattaagcttttgccagaaaatgttgcatgtgttttacctcgactt

SEQ ID NO: 614 - DNA, 523 bp, human
gaaaggcgatctcttcactgtgaaaagttgcccgggtgcagcgccttttccttctaccatgggaaatgcaggctgggcccttggggtgagcctgcggggctctggtgctgtccccgacccccaccaccaccagaatgcagttccagcttaggaagccacaaacaagccacccaggaggaacaaaacaccgccagcgtggattttccaaatttccctggaaagtaagtctcgctcttgccaaagaaaagtctggcttggagagtctctggagcccaggatgccagcatgtgccaatgactgtcaccttcatctcttcaaaagaaaagccatagccgaggactgtcccgcgacccccgtggactgcgtctaggtcatgtgattctgttttcatttctcatcccatccaatttgtccttttctcctgtcattttcttcctctgtggtcccttcaaagttgttataatttgtactgaacttcaaaatgtgtcccgttctccccagaccactctagccacagtatatt

SEQ ID NO: 615 - DNA, 542 bp, human
  misc_feature (56)..(56): n is a, c, g, or t
  misc_feature (63)..(63): n is a, c, g, or t
  misc_feature (102)..(102): n is a, c, g, or t
attgaaaagcaggtaccagtgccccttttcagacagtttttgattcgctctagacnttttttnttttttaatagggagggaaaaaatttgataattttcttntttctacatgcacttaagactaaaacacaggtttggattaattttatttgcttcctttttccgcttttcttcccgcagagcctgatgggagaatgtccagggcagggaaaccacattttttgtaggtgataactcaatgaaaattggtgcttattttttacacttctctcttgtggctctcttgtggtgctatctgttttaaggtctccttgaaggcgcactggggtccctggccatgcctcgttctccctgctttctttatcctgttattgcctccacagtctgttgccaaggactctaagatcaatgcacgtcactttcctttccactgggcaggatagccaagcacactccctcctgcgctctcccgccccggtgcgtccactcccgagggctgttatgaggactgggttgtgcctacttgatttgaaaacacacac

SEQ ID NO: 616 - DNA, 559 bp, human
  misc_feature (36)..(36): n is a, c, g, or t
  misc_feature (51)..(51): n is a, c, g, or t
  misc_feature (56)..(56): n is a, c, g, or t
acttctcagcaaataaatctcccttaagtaggaaanctagatttcatattngcttnctttgaattaacagcaactttccacaggtaaatctgttcttgcaaagatgtgagcagaatagttaaaaataatatttttatgtttcatggttctaaatggaagccataaatgcagtaaatactatctgttgtttaactactttaatcgtcattttttacattttcaagtttattaggttaagaaaaacagggcagccttggaaggcagctactacagaaaactgcagttttgcgttaaagataaagtagtattttcagctccctgaaaaaccattcctgctgaaactgctgtagaaattgtgaagctgcatgagtggagagtattgaatctgtggttatagtagttttctcaggtttgtttatcttgatgtttgatgcactgtgttttatagttattaaaattgagtaatattatttctatgcagtgttatgtgtcattggccttttgtgaatgtgcatgttttaaactgcaaattttaaacattttgtcctctaattgttat

SEQ ID NO: 617 - DNA, 462 bp, human
gaatgagtattcctatacgacccagatagactacaaccccaaggaccgcctgctctatgcctgggacaatggccaccaggtcacttaccatgtcatctttgcctactgacacccttgtccccacaagcagaagcacagaggggtcactagcaccttgtgtgtatgtgtgtgcgcgcacgtgtgtgtaggtgggtatgtgttgtttaaaaatatatattattttgtataatattgcaaatgtaaaatgacaatttgggtctatttttttatatggattgtagatcaatccatacgtgtatgtgctggtctcatcctccccagtttatatttttgtgcaaatgaacttctccttttgaccagtaaccaccttccttcaagccttcagcccctccagctccaagtctcagatctcgaccattgaaaaggtttcttcatctgggtcttgcaggaggcaggcaacac

SEQ ID NO: 618 - DNA, 503 bp, human
  misc_feature (419)..(419): n is a, c, g, or t
gtgctcggcctgatggagactgtgtgcagccccttcacggccgccagacgtctgcgggaccaggaggcagccgtggcggagctgcaggccgccctggagcgacaggctctccagaagcaagccctgcaggagaaaggcaagcagcaggacacggtcctcggcggccgggccctgtccaaccggcagcacgcctcctaggaactgtgggagaccagcggagtgggagggagacgcagtagacagagacagaccgagaaggaagggagagacagagggggcgcgcgcacaggagcctgactccgctgggagagtgcaggagcacgtgctgttttttatttggacttaacttcagagaaaccgctgacatctagaactgacctaccacaagcatccaccaaaggagtttgggattgagtttntgctgctgtgcagcactgcattgtcatgacatttccaacactgtgtgaattatctaaatgcgtctaccattttgcactagggag

SEQ ID NO: 619 - DNA, 568 bp, human
  misc_feature (82)..(82): n is a, c, g, or t
  misc_feature (251)..(251): n is a, c, g, or t
  misc_feature (393)..(393): n is a, c, g, or t
  misc_feature (407)..(407): n is a, c, g, or t
  misc_feature (412)..(412): n is a, c, g, or t
cacagcctccaagtcaggtggggtggagtcccagagctgcacagggtttggcccaagtttctaagggaggcacttcctcccntcgcccatcagtgccagcccctgctggctggtgcctgagcccctcagacagccccctgccccgcaggcctgccttctcagggacttctgcggggcctgaggcaagccatggagtgagacccaggagccggacacttctcaggaaatggcttttcccaacccccagcccnccacccggtggttcttcctgttctgtgactgtgtatagtgccaccacagcttatggcatctcattgaggacaaagaaaactgcacaataaaaccaagcctctggaatctgtcctcgtgtccacctggccttcgctcctccancagtgcctgcctgnccccncttcgctggggtctccacgggtgaggctggggaacgccacctcttcctcttccctgacttctccccaaccacttagtagcaacgctaccccaggggctaatgactgcacactgggcttcttttcagaatgaccctaacgagacacatttgcccaaa

SEQ ID NO: 620 - DNA, 248 bp, human
  misc_feature (45)..(45): n is a, c, g, or t
cacaaaaccccagggacagcggtctccccagcctgccctgctcangccttgcccccaaacctgtactgtcccggaggaggttgggaggtggaggcccagcatcccgcgcagatgacaccggttttcctagaagcccctcacccccactggcccactggtggctaggtctccccttatccttctggtccagcgcaaggaggggctgcttctgaggtcggtggctgtctttccattaaagaaacaccgtg

SEQ ID NO: 621 - DNA, 261 bp, human
gaatgaagaaaagtcgcctcaacgacaaacaaaagcaccgactagatttccttcagctgatgattgactcccagaattcgaaagaaactgagtcccacaaagctctgtctgatctggagctcgcagcccagtcaataatcttcatttttgctggctatgaaaccaccagcagtgttctttccttcactttatatgaactggccactcaccctgatgtccagcagaaactgcaaaaggagattgatgcagttttgcccaata

SEQ ID NO: 622 - DNA, 245 bp, human
ggtgaggggatgacccctggagatgaagggaagaggtgaagccttagcaaaaatgcctcctcaccactccccaggagaatttttataaaaagcataatcactgattccttcactgacataatgtaggaagcctctgaggagaaaaacaaagggagaaacatagagaacggttgctactggcagaagcataagatctttgtacaatattgctggccctggttcacctgtttactgttatcacaata

SEQ ID NO: 623 - DNA, 273 bp, human
acttaggtaattgtagggcgaggattataaatgaaatttgcaaaatcacttagcagcaactgaagacaattatcaaccacgtggagaaaatcaaaccgagcagggctgtgtgaaacatggttgtaatatgcgactgcgaacactgaactctacgccactccacaaatgatgttttcaggtgtcatggactgttgccaccatgtattcatccagagttcttaaagtttaaagttgcacatgattgtataagcatgctttctttgagttttaaat

SEQ ID NO: 624 - DNA, 212 bp, human
  misc_feature (43)..(43): n is a, c, g, or t
  misc_feature (121)..(121): n is a, c, g, or t
tctcacctatacgtgcaagaaaggaacagttgcttgcggccancccctgttgtagaaaatgccaagacctttggaaagatgaaacctcgttatgaaatcaactccctgattagataccacntgcaaagatggtttcattcaacgtcaccttccaactatccggtgcttaggaaatggaagatgggctatacctaaaattacctgcatgaacc

SEQ ID NO: 625 - DNA, 157 bp, human
agggcctaagggtgacagaggtgatgctggtcccaaaggtgctgatggctctcctggcaaagatggcgtccgtggtctgaccggccccattggtcctcctggccctgctggtgcccctggtgacaagggggaccccattcccgaggagctttatgag

SEQ ID NO: 626 - DNA, 481 bp, human
aacattgtaatggccatcgctggaaacaagtgcgacctctcagatattagggaggttcccctgaaggatgctaaggaatacgctgaatccataggtgccatcgtggttgagacaagtgcaaaaaatgctattaatatcgaagagctctttcaaggaatcagccgccagatcccacccttggacccccatgaaaatggaaacaatggaacaatcaaagttgagaagccaaccatgcaagccagccgccggtgctgttgacccaagggcgtggtccacggtacttgaagaagccagagcccacatcctgtgcactgctgaaggaccctacgctcggtggcctggcacctcactttgagaagagtgagcacactggctttgcatcctggaaggcctgcagggggcggggcaggaaatgtacctgaaaaggattttagaaaaccctgggaaacccaccacaccaccacaaaatggcctttagtgt

SEQ ID NO: 627 - DNA, 399 bp, human
gccacaccttcgcgaaacctgtggtggcccaccagtcctaacgggacaggacagagagacagagcagccctgcactgttttccctccaccacagccatcctgtccctcattggctctgtgctttccactatacacagtcaccgtcccaatgagaaacaagaaggagcaccctccacatggactcccacctgcaagtggacagcgacattcagtcctgcactgctcacctgggtttactgatgactcctggctgccccaccatcctctctgatctgtgagaaacagctaagctgctgtgacttccctttaggacaatgttgtgtaaatctttgaaggacacaccgaagacctttatactgtgatcttttacccctttcactcttggctttcttatgttgc

SEQ ID NO: 628 - DNA, 548 bp, human
ctgccccgagctcgaagaagaggctgagtgcgtccctgataactgcgtctaagaccagagccccgcagcccctggggcccccggagccatggggtgtcgggggctcctgtgcaggctcatgctgcaggcggccgaggcacagggggtttcgcgctgctcctgaccgcggtgaggccgcgccgaccatctctgcactgaagggccctctggtggccggcacgggcattgggaaacagcctcctcctttcccaaccttgcttcttaggggcccccgtgtcccgtctgctctcagcctcctcctcctgcaggataaagtcatccccaaggctccagctactctaaattatggtctccttataagttattgctgctccaggagattgtccttcatcgtccaggggcctggctcccacgtggttgcagatacctcagacctggtgctctaggctgtgctgagcccactctcccgagggcgcatccaagcgggggccacttgagaagtgaataaatggggcggtttcggaagcgtcagtgtttccatgttatgg

SEQ ID NO: 629 - DNA, 524 bp, human
gaagttgcaacattcgtttgataggaattccagaaaaggagagttatgagaatagggcagaggacataattaaagaaataattgatgaaaactttgcagaactaaagaaaggttcaagtcttgagattgtcagtgcttgtcgagtacctagtaaaattgatgaaaagagactgactcctagacacatcttggtgaaattttggaattctagtgataaagagaaaataataagggcttctagagagagaagagaaattacctaccaaggaacaagaatcaggttgacagcagacttatcactggacacactggatgctagaagtaaatggagcaatgtcttcaaagttctgctggaaaaaggctttaatcctagaatcctatatccagccaaaatggcatttgattttaggggcaaaacaaaggtatttcttagtattgaagaatttagagattatgttttgcatatgcccaccttgagagaattactggggaataatataccttagcacgccagggtgactaca

SEQ ID NO: 630 - DNA, 488 bp, human
gagtggttcatccatactctcattccctcgcctccccttgtggacgggggtcttgccttttcaattcctgtgttttggtgtcttcccttatctgctaccctgaatcacctgtcctggtcttgctgtgtgatgggaacatgcttgtaaactgcgtaacaaatctactttgtgtatgtgtctgtttatgggggtggtttattatttttgctggtccctagaccactttgtatgaccgtttgcagtctgagcaggccaggggctgacagctaatgtcaggaccctcagcggtggagcctgctggggggacccagctgctcttggacaagtggctgagctcctatctggcctcctcttttttttttttttcaagtaatttgtgtgtatttctaactgattgtattgaaaaaattcctagtatttcagtaaaaatgcctgttgtgagatgaacctcctgtaacttctatctgttcttttttgaggctcaggga

SEQ ID NO: 631 - DNA, 460 bp, human
tggaattagaccatttggcctttgaactttcataggaaaaatgacccaacatttcttagcatgagctacctcatctctagaagctgggatggacttactattcttgtttatattttagatactgaaaggtgctatgcttctgttattattccaagactggagataggcagggctaaaaaggtattattatttttcctttaatgatggtgctaaaattcttcctataaaattccttaaaaataaagatggtttaatcactaccattgtgaaaacataactgttagacttcccgtttctgaaagaaagagcatcgttccaatgcttgttcactgttcctctgtcatactgtatctggaatgctttgtaatacttgcatgcttcttagaccagaacatgtaggtccccttgtgtctcaatactttttttttcttaattgcatttgttggctctattttaattt

SEQ ID NO: 632 - DNA, 446 bp, human
tagattccggtatatcgttcttcaagacacttgctctaagcggaatggaaatgtgggcaagactgtctttgaatatagaacacagaatgtggcacgcttgcccatcatagatcttgctcctgtggatgttggcggcacagaccaggaattcggcgttgaaattgggccagtttgttttgtgtaaagtaagccaagacacatcgacaatgagcaccaccatcaatgaccaccgccattcacaagaactttgactgtttgaagttgatcctgagactcttgaagtaatggctgatcctgcatcagcattgtatatatggtcttaagtgcctggcctccttatccttcagaatatttattttacttacaatcctcaagttttaattgattttaaatatttttcaatacaacagtttaggtttaagatgaccaatgacaatgaccacctt

SEQ ID NO: 633 - DNA, 552 bp, human
  misc_feature (63)..(63): n is a, c, g, or t
  misc_feature (72)..(72): n is a, c, g, or t
  misc_feature (74)..(74): n is a, c, g, or t
  misc_feature (85)..(85): n is a, c, g, or t
  misc_feature (138)..(138): n is a, c, g, or t
tttcagcaccgatggccatgtaaataagatgatttaatgttgattttaatcctgtatataaantaaaaagtncncaatgagtttngggcatatttaatgatgattatggagccttagaggtctttaatcattggttcnggctgcttttatgtagtttaggctggaaatggtttcacttgctctttgactgtcagcaagactgaagatggcttttcctggacagctagaaaacacaaaatcttgtaggtcattgcacctatctcagccataggtgcagtttgcttctacatgatgctaaaggctgcgaatgggatcctgatggaactaaggactccaatgtcgaactcttctttgctgcattcctttttcttcacttacaagaaaggcctgaatggaggacttttctgtaaccaggaacattttttaggggtcaaagtgctaataattaactcaaccaggtctactttttaatggctttcataacactaactcataaggttaccgatcaatgcatttcatacggatatagacctagggctctggagggtgggg

SEQ ID NO: 634 - DNA, 440 bp, human
  misc_feature (165)..(165): n is a, c, g, or t
ctggcctacgtgttcaattttctatgaacaaaggctttagtccttgacccagggctaaagtggtctgtccaagctgttgttggtagagggagtatgataaaatgtttaaatctcatttggttaccttgagtcctggaacacgcagtaactgtcatgctatagtcntcatctgtatttggctgggaatacaaatgaagattgtggtgtattcaagcagtagggtttttgcttttgtttttgttttagtgccaacaaaacttttttttgtctgactacattaaagataagactgactatatttatacaacagaaactttgtaatagattttttcagctttgtgaaatcgaattttttttcatcagggctggttggatttcctttttaccctgtaatccaagcgttaatagtttgttagaagatgggttattgcatgtcactt

SEQ ID NO: 635 - DNA, 360 bp, human
gaagatcaaggatgttctcaacagtctagagtacagtccctctcctataagcaagaagctctcgtgtgctagtgtcaaaagccaaggcagaccgtcctcctgccctgctgggatggctgtcactggctgtgcttgtggctatggctgtggttcgtgggatgttcagctggaaaccacctgccactgccagtgcagtgtggtggactggaccactgcccgctgctgccacctgacctgacagggaggaggctgagaactcagttttgtgaccatgacagtaatgaaaccagggtcccaaccaagaaatctaactcaaacgtcccacttcatttgttccattcctgattcttgggtaataaa

SEQ ID NO: 636 - DNA, 490 bp, human
ggagctcagagatctaagctgctttccatcttttctcccagccccaggacactgactctgtacaggatggggccgtcctcttgcctccttctcatcctaatcccccttctccagctgatcaacccggggagtactcagtgttccttagactccgttatggataagaagatcaaggatgttctcaacagtctagagtacagtccctctcctataagcaagaagctctcgtgtgctagtgtcaaaagccaaggcagaccgtcctcctgccctgctgggatggctgtcactggctgtgcttgtggctatggctgtggttcgtgggatgttcagctggaaaccacctgccactgccagtgcagtgtggtggactggaccactgcccgctgctgccacctgacctgacagggaggaggctgagaactcagttttgtgaccatgacagtaatgaaaccagggtcccaaccaagaaatctaactcaaacgtcccact

SEQ ID NO: 637 - DNA, 577 bp, human
gcctgagaaagcaagcacgcactctcagtcaacatgacagattctggaggataaccagcaggagcagagataacttcaggaagtccatttttgcccctgcttttgctttggattatacctcaccagctgcacaaaatgcattttttcgtatcaaaaagtcaccactaaccctcccccagaagctcacaaaggaaaacggagagagcgagcgagagagatttccttggaaatttctcccaagggcgaaagtcattggaatttttaaatcataggggaaaagcagtcctgttctaaatcctcttattcttttggtttgtcacaaagaaggaactaagaagcaggacagaggcaacgtggagaggctgaaaacagtgcagagacgtttgacaatgagtcagtagcacaaaagagatgacatttacctagcactataaaccctggttgcctctgaagaaactgccttcattgtatatatgtgactatttacatgtaatcaacatgggaacttttaggggaacctaataagaaatcccaattttcaggagtggtggtgtcaataaacgctctgtggccagtg

SEQ ID NO: 638 - DNA, 351 bp, human
ggaacccagagctgctgtgtatttcgagcgggcagtttatcttttgctatacttattttcaattcaattacaccacgattcaaataattcccctcctaaaaccaaaaaggagggaaacgtcaactccattgcaattacttatcttcctcttctatctctgttatacgccggggcatagaatgctcgtatacatctctttaacaaccacaaaccttaagccatgtagatgaagttagtgcatcaacgggatacagttccatattgccttaaacctccttgttttagacacactaacatttataccaaattgcagattattctgcagagagggaattgcatgtttgtgttgta

SEQ ID NO: 639 - DNA, 506 bp, human
aattaatattcatcgcacttcttctgtggaaggactttgtgaaggaattggtgctggattagtggatgttgctatctgggttggcacttgttcagattacccaaaaggagatgcttctactggatggaattcagtttctcgcatcattattgaagaactaccaaaataaatgctttaattttcatttgctacctctttttttattatgccttggaatggttcacttaaatgacattttaaataagtttatgtatacatctgaatgaaaagcaaagctaaatatgtttacagaccaaagtgtgatttcacactgtttttaaatctagcattattcattttgcttcaatcaaaagtggtttcaatattttttttagttggttagaatactttcttcatagtcacattctctcaacctataatttggaatattgttgtggtcttttgttttttctcttagtatagcatttttaaaaaaatataaaagctaccaatctttgtacaatttg

SEQ ID NO: 640 - DNA, 360 bp, human
  misc_feature (128)..(128): n is a, c, g, or t
  misc_feature (190)..(190): n is a, c, g, or t
  misc_feature (206)..(206): n is a, c, g, or t
aaatgactggatggtcgctgctttttaagtttcaaattgacattccagacaagcggtgcctgagcccgtgcctgtcttcagatcttcacagcacagttcctgggaaggtggagccaccagcctctccntgaataactgggagatgaaacaggaagctctatgacacacttgatcgaatatgacagacacngaaaatcacgactcanccccctccagcacctctacctgttgcccgccgatcacagccggaatgcagctgaaagattccctggggcctggttccaaccgcccactgtggactctgaggcctctgcatttgcgggtggtctgcctgtgatattttggtcatgggctggtctg

SEQ ID NO: 641 - DNA, 427 bp, human
gaagaggagcaacatctatgccaaatactgtgcattctacaatggtgctaatctcagacctaaatgatactccatttaatttaaaaaagagttttaaataattatctatgtgcctgtatttcccttttgagtgctgcacaacatgttaacatattagtgtaaaagcagatgaaacaaccacgtgttctaaagtctagggattgtgctataatccctatttagttcaaaattaaccagaattcttccatgtgaaatggaccaaactcatattattgttatgtaaatacagagttttaatgcagtatgacatcccacaggggaaaagaatgtctgtagtgggtgactgttatcaaatattttatagaatacaatgaacggtgaacagactggtaacttgtttgagttcccatgacagatttgagacttg

SEQ ID NO: 642 - DNA, 574 bp, human
  misc_feature (83)..(83): n is a, c, g, or t
  misc_feature (94)..(94): n is a, c, g, or t
  misc_feature (103)..(103): n is a, c, g, or t
  misc_feature (134)..(138): n is a, c, g, or t
  misc_feature (140)..(141): n is a, c, g, or t
  misc_feature (143)..(143): n is a, c, g, or t
  misc_feature (145)..(146): n is a, c, g, or t
  misc_feature (169)..(169): n is a, c, g, or t
  misc_feature (171)..(172): n is a, c, g, or t
  misc_feature (174)..(177): n is a, c, g, or t
  misc_feature (179)..(179): n is a, c, g, or t
  misc_feature (181)..(181): n is a, c, g, or t
  misc_feature (183)..(184): n is a, c, g, or t
  misc_feature (186)..(193): n is a, c, g, or t
  misc_feature (196)..(196): n is a, c, g, or t
  misc_feature (198)..(203): n is a, c, g, or t
  misc_feature (206)..(207): n is a, c, g, or t
tgaaagtgggcatctctgcaggcacctgtactgccatcctgctcaccgtcttgacctgctacttttggaaaaagaatcaaaanctagagtacangtactccangctggtgatgaatgctactctcaaggactgnnnnntnncngnngctgacagctgcgccatcatggnanncnnnnangnanngnnnnnnnncancnnnnnncanntgcgccatcatggaaggcgaggatgtagaggacgacctcatctttaccagcaagaagtcactctttgggaagatcaaatcatttacctccaagaggactcctgatggatttgactcagtgccgctgaagacatcctcaggaggcccagacatggacctgtgagaggcactgcctgcctcacctgcctcctcaccttgcatagcacctttgcaagcctgcggcgatttgggtgccagcatcctgcaacacccactgctggaaatctcttcattgtggccttatcagatgtttgaatttcagatctttttttatagagtacccaaaccctcctttctgcttgcctcaaacctgccaaatatacccacac

SEQ ID NO: 643 - DNA, 485 bp, human
aaacgacgcaaatctctgagctggggaccacttggagaaccggcttagtaacagtcctgatcttcgcaagccagcttcttctgcatctgaggggctcctggcgcccagaggaggcagacagatgtcttctagctgagtttctaaccgcatgatgagactcagaccttccgctgcactagaaaatctgcaacagtgtccctgagtcacttctccttagtgggcagactcgtgttagatttgtggaacccagctctctgatttactccttttggaaaacccatggaatttcatgtataaggctttcatttgtattttaaggtttttctgtttgttttgagtatatacatggtgctcaatagcaacatcttagcagatgaagcagtttatgattccactccctcctgtatgacaggtagccactatactgaatcaaggtgctgaactcaaatcacaaaattctggcttaccgatacaacaaccaatac

SEQ ID NO: 644 - DNA, 487 bp, human
  misc_feature (296)..(296): n is a, c, g, or t
tataaggtaactctttagtcctccatttagcacattttaaatcctccaaagaataagtatcatgtgattattttagctttacaaaaaaaaagttgaatggcgttttattttcatggcctataagcaggtaccttagtagggcagatataggaaaaacaaattagagcaaaacaaatcctctacaaatccaaggcaggaaaagtggtggcagagtgactcattctcctgtccctcccatcaggtcaaatcaggaggctgcagtgaatgcctgttctttgaatgtgtagcagttgttncctgtaactctttaaaacttggctataggctgtttagcacagtacagattaaagatacagttacgtaaacagcaaagtaattttatagtgcttcatccatttatcatgctttggtttgctaattttttcacatacctttttctatcacagtctgttgcttttgtacacatttctcatattggggttcgaca

SEQ ID NO: 645 - DNA, 520 bp, human
ggtgccaggaaggtcacagatggacactggccattctggtcatctcagtctggaactcagtcccacttcttggcctggacaatgaacaggattcagttttgctgttaactttgcttctctacttttttttgtttgtttgtaatagcacatcccagagacatcagaaaccagcaactgattcagtgtgatttccagactttttaggcatgaaattcggacacttcagtatttccaggaatagcatatgcacgctgttcttgcttcatggaatgctacatgctttctgtttttctcattttggatttctccaaaactaactgaatttaagcttcaggtccctttgtatgcagtagaaaggaattattaaaaacaccaccaaagaaaataaatatatcctacttgaaatttactctatggacttacccactgctagaataaatgtatcaaatcttatttgtaaattctcaattttgatatatatatgtatatatgcatatacatatccacacttgtctgcaag

SEQ ID NO: 646 - DNA, 516 bp, human
ttaccctctatttaaatgctttgaaaaacagtgcattgacaatgggttgatatttttctttaaaagaaaaatataattatgaaagccaagataatctgaagcctgttttattttaaaactttttatgttctgtggttgatgttgtttgtttgtttgtttctattttgttggttttttactttgttttttgttttgttttgttttgttttgcatactacatgcagttctttaaccaatgtctgtttggctaatgtaattaaagttgttaatttatatgagtgcatttcaactatgtcaatggtttcttaatatttattgtgtagaagtactggtaatttttttatttacaatatgtttaaagagataacagtttgatatgttttcatgtgtttatagcagaagttatttatttctatggcattccagcggatattttggtgtttgcgaggcatgcagtcaatattttgtacagttagtggacagtattcagcaacgcctgatagcttctttggcctt

SEQ ID NO: 647 - DNA, 390 bp, human
ccacacaccaggcactaatcacctggtgaggatttggcatatccaccaaaaaatgcatccgatttaaccaacatctccaccagcgctacggactcctcccaattctgacatctcttgcagacaatactatgctctctacacactgtttagaaatggaaaggtgatctgcactgtatcttgggtttgttggctatgcttcctttgatgacatatattatacagtatatatatacatatattttttttgttagagttctagccattttatttctccgcagggtcctttctcagacattactgcatgctgtatatggcgttagctgtgtgttgatcttctaaaagatgatagagtttactggtaattgtgtaatcagctcctgcctttttatt

SEQ ID NO: 648 - DNA, 505 bp, human
  misc_feature (39)..(39): n is a, c, g, or t
caggaaactcttctctgacttggggagttcctacgcganacaactgggcttccgggacagctgggtcttcataggagccaaagacctcaggggtaaaagcccctttgagcagttcttaaagaacagcccagacacaaacaaatacgagggatggccagagctgctggagatggagggctgcatgcccccgaagccattttagggtggctgtggctcttcctcagccaggggcctgaagaagctcctgcctgacttaggagtcagagcccggcaggggctgaggaggaggagcagggggtgctgcgtggaaggtgctgcaggtccttgcacgctgtgtcgcgcctctcctcctcggaaacagaaccctcccacagcacatcctacccggaagaccagcctcagagggtccttctggaaccagctgtctgtggagagaatggggtgctttcgtcagggactgctgacggctggtcctgaggaaggacaaactgcccagacttgagcc

SEQ ID NO: 649 - DNA, 331 bp, human
  misc_feature (73)..(74): n is a, c, g, or t
  misc_feature (76)..(76): n is a, c, g, or t
cacttgatagactgtaagcacctgcttaactttgtgtcccaaatatttagtgtgtatatatatatatatatanncncacacacacacatatatattcaacaaataaagcaaaatataacatgcatttcacattttgtctttccctgttacgattttaatagcagaactgtatgacaagtttaggtgatcctagcatatgttaaattcaaattaatgtaaaacagattaacaacaacaaagaaactgtctatttgagtgaagtcatgctttctattataataacttggcttcggttatccatcaaatgcacacttatactgttatctgattg

SEQ ID NO: 650 - DNA, 582 bp, human
  misc_feature (179)..(180): n is a, c, g, or t
  misc_feature (218)..(218): n is a, c, g, or t
  misc_feature (226)..(227): n is a, c, g, or t
gccaaaaggcctggcttctgataggaaactggtaagaaactcttcatgaaaacacatcactaatattcgctattactctcctggtctgaagtcagcttttctgaaccattaaggtatttcatcacaagttatattttataatatcagtttaagaggcttttattcatgtgaacaccagnnccctttcaggggcatggtctttttgaaaaaaaaaaaanaaaaaaannaacagttttagccacatatcagatatttctatatctaattatcctttatggctaacattctgcctccattgttaaggtataattgttcctgaatttaaaggtggtttggcctctaatttaattctgattcagactctcctgtcaggactcaagaaaatttaattaattaccaaggattaagtcttctggttaaggtttctgggaaaaaaaaatagcaaagatgttgatttcttggaatccttttacaggttcataacagaaaaatcttcattccctgtaggcatttaattaaacctagttgagaagtgtgtgggattcctcaattatgaacaaaacacgtatattggctttcttt

SEQ ID NO: 651 - DNA, 433 bp, human
gacatgattgtctataatctcgctagccttgtactgtgtgtgcatagcaattacagggaagtaatctagctcctgactattatgttgaactatgtcgctgctttttacaaacttgtcttgatccaaagcagtcacaatgataaccctgcatatctgggaatcataagtcaactatgtatctctgtgtgtgtatatatatgtatgtatgtatctattttcaaactgtgatttaatatttaaatattcctactgccatttttgtgactgaaaaactacacatgaggaaacgtcttagaattttccaatagaggaaaaataacacttgggcaatctgtcatgtttcacaacagttctcatttttctcatgatttgtgtagcgtggaatgtgtttgctcaatgtgaagggttttcattgctcaatttctctgtgtaa

SEQ ID NO: 652 - DNA, 361 bp, human
  misc_feature (71)..(71): n is a, c, g, or t
  misc_feature (295)..(304): n is a, c, g, or t
ggttccggctaacacattttctaagtcgccagtgctgcttacagtttgaatacatgaaaatcctgtttctnagatgtttgcgcacgtgcttattaggaaatgagtctgtatggaaatctcaccacagataatggttaacgaaccgggtcgacatcacaaaggagggtggagactctttttactaacttgaatgagacaaaagcagtggtgtcagtttataatcctgatgcatttcagtaataatgtagaaaaacattattttaaaaaagttccaacacacagccatgaggagccnnnnnnnnnntcagttttgaaagaggtgcataataaaactactaaccagaggagtctatgccatttt

SEQ ID NO: 653 - DNA, 415 bp, human
  misc_feature (42)..(42): n is a, c, g, or t
  misc_feature (95)..(95): n is a, c, g, or t
  misc_feature (353)..(353): n is a, c, g, or t
gagtttcaactttaaatgttcactatgtcatttagtgtccanctttacggataggttgactatctaaataggcatttttagtcattaaaaaaaantctagtcaccaggaggatccctataactcaaaataacttgtttgtaaaagaaaatttgtttacttacccattagtaagttcctgcatattcattataagatggcaaatcaaacttttctaggatgaagacagcttatttttaagttgtatagtcttagttggtttagggtctcaattttaattaataaaatacttggtttttatttgcttgtccttttgaattcctgttttaataattttaaaatgagcacaaagaangttgaagttcagattaatctcttctgaatgatgtttttttcctctgtgatgagttgtttctg

SEQ ID NO: 654 - DNA, 364 bp, human
  misc_feature (282)..(282): n is a, c, g, or t
  misc_feature (294)..(294): n is a, c, g, or t
  misc_feature (336)..(337): n is a, c, g, or t
tgctgtagtcactggtgttcctcacccaccagctgtaactcagtttgtgtgaggtacagccacagaagatgtcatgtactgtatattacctggtgatagttgcttttcaccccccgagttcagtttctaggagccaatgaaacttcccctcacctcctcatctttccaagttgttctttgaattgaggagtttgaaggcataaacagttacttggggatttgcgaaaatcctacttagttactgcgtttacagttctttggcccagtctctgacccttcccnagtatttgtgcnatgattgtgtttactgctggatttttgaaggttttttttttnnaagaaagtgccatttcattatttgatt

SEQ ID NO: 655 - DNA, 436 bp, human
gggatcactgggagaagccatggcattatcttcaggcaatttagtctgtcccaaataaaataaatccttgcatgtaaatcattcaagggttatagtaatatttcatatactgaaaagtgtctcataggagtcctcttgcacatctaaaaaggctgaacatttaagtatcccgaattttcttgaattgctttccctatagattaattacaattggatttcatcatttaaaaaccatacttgtatatgtagttataatatgtaaggaatacattgtttataaccagtatgtacttcaaaaatgtgtattgtcaaacatacctaactttcttgcaataaatgcaaaagaaactggaacttgacaattataaatagtaatagtgaagaaaaaatagaaaggttgcaattatataggccatgggtggctcaaaactttgaa

SEQ ID NO: 656 - DNA, 427 bp, human
  misc_feature (45)..(51): n is a, c, g, or t
  misc_feature (402)..(402): n is a, c, g, or t
gattcctggattgatgtcttatctacaggcttctttttaaaaatnnnnnnngattggctgtgagtttaccaggattatagttgaggctaaaggacagctcctcaggaaagcccctgttctacatctacggtcacatgctggaccttgagttgtcactcagagaaaagagtgccatctaccgaaactccacagtttccattgtgaatggcttctttggtgcagagttccaaaaattatgtagcccagctctttaattttgtaacatctaatgatatcaccgccttgaagtgattaaagtagattgcttaaagaattaaagctttaaagatgaaagatgttattgcttttgctggacatgaggaacagttgtaaagtttccaggtctacaataactttctgganccctctcagtgaactgtttcttgta

SEQ ID NO: 657 - DNA, 559 bp, human
gaagtccatcctttggtccaaagcatctggaagaggaagaagagaggaatgagaaagaaggaagtgatgcaaaacatctccaaagaagtcttttggaacaggaaaatcattcaccactcacagggtcaaatatgaaatacaaaaccacgaaccaatcaacagaatttttatccttccaagatgccagctcattgtacagaaacattttagaaaaagaaagggaacttcagcaactgggaatcacagaatacctaaggaaaaacattgctcagctccagcctgatatggaggcacattatcctggagcccacgaagagctgaagttaatggaaacattaatgtactcacgtccaaggaaggtattagtggaacagacaaaaaatgagtattttgaacttaaagctaatttacatgctgaacctgactatttagaagtcctggagcagcaaacatagatggagagtttgagggctttcgcagaaatgctgtgattctgttttaagtccataccttgtaaataagtgccttacgtgagtgtgtcatcaatcagaacctaagc

SEQ ID NO: 658 - DNA, 368 bp, human
gctgctcaagcgcctgcagaacaacgacacgtgcagcatgccaggcctcacgtgcttcacccacgacaaccagcactggcagacggcgcctttctggacactggggcctttctgtgcctgcaccagcgccaacaataacacgtactggtgcatgaggaccatcaatgagactcacaatttcctcttctgtgaatttgcaactggcttcctagagtactttgatctcaacacagacccctaccagctgatgaatgcagtgaacacactggacagggatgtcctcaaccagctacacgtacagctcatggagctgaggagctgcaagggttacaagcagtgtaacccccggactcgaaacatggacctgg

SEQ ID NO: 659 - DNA, 436 bp, human
  misc_feature (300)..(300): n is a, c, g, or t
acaatcggctaaccttgacatttctttttaccttcatatgccactatctcggtagttcaaaaaaatttagttcttgataaattgccttgaagtttaccttgtgctggagagccttatgataactccaaagactttcttacggtataatacatgttgtttaggattgtgtttcttagtcactgaagataataaatattaaaatggatgttttcatcagaaaattttcatgttttcctttaaggtaacataattgtaagaattgtttaataaaatactcaggaaattctaaaggtttctccnaatacctaaacatttctgaacatcagtattgcagttgtggaagagcagaaggaggatacatttgtttgtgttgctccccaaaattccaccttgcatttgcatcacaaacttccctcaattgaggcagttttctttg

SEQ ID NO: 660 - DNA, 431 bp, human
  misc_feature (311)..(311): n is a, c, g, or t
  misc_feature (391)..(391): n is a, c, g, or t
cagtgctgctgtgaactaaagtatgtcatttatgctcaaagtttaattcttcttcttgggatattttaaaaatgctactgagattctgctgtaaatatgactagagaatatattgggtttgctttatttcataggcttaattctttgtaaatctgaatgaccataatagaaatacatttcttgtggcaagtaattcacagttgtaaagtaaataggaaaaattattttatttttattgatgtacattgatagatgccataaatcagtagcaaaaggcacttctaaaggtaagtggtttaagttgcctcaanagagggacaatgtagctttattttacaagaaggcatagttagatttctatgaaatatttattctgtacagttttatatanttttggttcacaaaagtaattattcttgggtgcctttcaa

SEQ ID NO: 661 - DNA, 322 bp, human
  misc_feature (37)..(38): n is a, c, g, or t
aaagtatattgtgctagcttgtctaagaataaacttnnatactgttgggggagggctgcacctgtcaagataacctgtcaatgtagtaggaaaacaggaggggacagtaacagaaaagcacgggaaaagatggcaaggttagttaaaatagaaaagtgctcagttcctcatacctgtaatcccagcagtttagggggccaaggaaggtgggtcacttgagcccaagagttcaaggccatcctgggcaatgtggcgaaagtgtctacaaaaaaatacaaaaagaggaagaaatgatatttcacaagtttgtatcatttgtcat

SEQ ID NO: 662 - DNA, 282 bp, human
  misc_feature (43)..(43): n is a, c, g, or t
  misc_feature (50)..(50): n is a, c, g, or t
  misc_feature (99)..(99): n is a, c, g, or t
  misc_feature (115)..(115): n is a, c, g, or t
cagtccaaaatccatagacacagacagtagaaaggtggttatncaagggnctggtaggagagggagttagtatttagtggtatagagttttagtgtttnccgggtaaaaatgttnctagagatctgttgcaaccatgtgaatatacttaacactaccgtactgtacactgaaaaaatggttaagaggataaatttaaatggttaagaggaaaaatggttaagaggataatgttgttttgttaccacagtagtaacttttaaaaaccctcatgtatgttcttt

SEQ ID NO: 663 - DNA, 398 bp, human
  misc_feature (80)..(80): n is a, c, g, or t
  misc_feature (82)..(83): n is a, c, g, or t
  misc_feature (85)..(85): n is a, c, g, or t
  misc_feature (87)..(87): n is a, c, g, or t
  misc_feature (97)..(97): n is a, c, g, or t
caacccattttgtgccacatgcaagttttgaataaggatggtatagaaaacaacgctgcatatacaggtaccatttaggnannancngatgcctttntgggggcagaatcacatggcaaaagctttgaaaatcataaagatataagttggtgtggctaagatggaaacagggctgattcttgattcccaattctcaactctccttttcctatttgaatttctttggtgctgtagaaaacaaaaaaagaaaaatatatattcataaaaaatatggtgctcattctcatccatccaggatgtactaaaacagtgtgtttaataaattgtaattattttgtgtacagttctatactgttatctgtgtccatttccaaaacttgcacgtgtccctgaattcc

SEQ ID NO: 664 - DNA, 22 bp, Artificial sequence
  Synthetic Oligonucleotide
tcgtttttcgcgtttagttgtc

SEQ ID NO: 665 - DNA, 17 bp, Artificial sequence
  Synthetic Oligonucleotide
gcgcaccgaaaaaaccg

SEQ ID NO: 666 - DNA, 24 bp, Artificial sequence
  Synthetic Oligonucleotide
cggttacggttagtattcggtttc

SEQ ID NO: 667 - DNA, 23 bp, Artificial sequence
  Synthetic Oligonucleotide
aaaatacgacgataaaaaacgcg

SEQ ID NO: 668 - DNA, 23 bp, Artificial sequence
  Synthetic Oligonucleotide
cgagggttttttagcgatttgtc

SEQ ID NO: 669 - DNA, 17 bp, Artificial sequence
  Synthetic Oligonucleotide
aaacgaaccgctccccg

SEQ ID NO: 670 - DNA, 21 bp, Artificial sequence
  Synthetic Oligonucleotide
ttcggcgttttcgttttttac

SEQ ID NO: 671 - DNA, 21 bp, Artificial sequence
  Synthetic Oligonucleotide
ccccttaacaacataatcgcg

SEQ ID NO: 672 - DNA, 23 bp, Artificial sequence
  Synthetic Oligonucleotide
gatggtgcgttcggtatttatgt

SEQ ID NO: 673 - DNA, 19 bp, Artificial sequence
  Synthetic Oligonucleotide
gcttacacccgcccgacta

SEQ ID NO: 674 - DNA, 28 bp, Artificial sequence
  Synthetic Oligonucleotide
gttttgtgattggttaggttattaaagt

SEQ ID NO: 675 - DNA, 25 bp, Artificial sequence
  Synthetic Oligonucleotide
ccrtcaccttctacaccaaaaaata

SEQ ID NO: 676 - DNA, 30 bp, Artificial sequence
  Synthetic Oligonucleotide
tgtggatttatttttataaggatattgaat

SEQ ID NO: 677 - DNA, 32 bp, Artificial sequence
  Synthetic Oligonucleotide
ataactcattaaaaactcacatctaaacacta

SEQ ID NO: 678 - DNA, 28 bp, Artificial sequence
  Synthetic Oligonucleotide
ttaagaaagagagtaaggaggaagagtt

SEQ ID NO: 679 - DNA, 24 bp, Artificial sequence
  Synthetic Oligonucleotide
aaaatcccaattaaaaaacaacca

SEQ ID NO: 680 - DNA, 33 bp, Artificial sequence
  Synthetic Oligonucleotide
caaaataataaatccctctactactattatcaa

SEQ ID NO: 681 - DNA, 30 bp, Artificial sequence
  Synthetic Oligonucleotide
ggaggaaaggaattagtttagaagttagtt

(19) United States
(12) Patent Application Publication
(10) Pub. No.: US 2019/0024188 A1
(43) Pub. Date: Jan. 24, 2019

(54) METHOD OF DIAGNOSING NEOPLASMS - II

(71) Applicants: CLINICAL GENOMICS PTY. LTD., NEW SOUTH WALES (AU); COMMONWEALTH SCIENTIFIC AND INDUSTRIAL RESEARCH ORGANISATION, AUSTRALIAN CAPITAL TERRITORY (AU)

(72) Inventors: Lawrence C. LAPOINTE, West Pennant Hills (AU); Robert DUNNE, Darlington (AU); Graeme P. YOUNG, Malvern (AU); Trevor John LOCKETT, Denistone (AU); William J. WILSON, Hornsby Heights (AU); Peter Laurence MOLLOY, Chatswood (AU)

(73) Assignees: CLINICAL GENOMICS PTY. LTD., NEW SOUTH WALES (AU); COMMONWEALTH SCIENTIFIC AND INDUSTRIAL RESEARCH ORGANISATION, AUSTRALIAN CAPITAL TERRITORY (AU)

(21) Appl. No.: 16/149,653

(22) Filed: Oct. 2, 2018

Related U.S. Application Data
(63) Continuation of application No. 14/057,812, filed on Oct. 18, 2013.
Application No. 14/057,812 is a division of application No. 12/739,540, filed on Nov. 19, 2010, now abandoned.
(60) Provisional application No. 60/982,115, filed on Oct. 23, 2007.

(51) Int. Cl. C12Q 1/6886

(52) U.S. Cl. CPC C12Q 1/6886; C12Q 2600/154; C12Q 2600/112

(57) ABSTRACT

The present invention relates generally to nucleic acid molecules in respect of which changes to the DNA or to the RNA or protein expression profiles are indicative of the onset, predisposition to the onset and/or progression of a neoplasm. More particularly, the present invention is directed to nucleic acid molecules in respect of which changes to the DNA or to the RNA or protein expression profiles are indicative of the onset and/or progression of a large intestine neoplasm, such as a adenoma or an adeocarcinoma. The DNA or the expression profiles of the present invention are useful in a range of applications including, but not limited to, those relating to the diagnosis and/or monitoring of colorectal neoplasms, such as colorectal adenocarcinoma. Accordingly, in a related aspect the present invention is directed to a method of screening a subject for the onset, predisposition to the onset and/or progression of a neoplasm by screening for modulation in thin DNA or the RNA or protein expression profile of one or more nucleic acid molecule markers.

### FIELD OF THE INVENTION

The present invention relates generally to nucleic acid molecules in respect of which changes to the DNA or to the RNA or protein expression profiles are indicative of the onset, predisposition to the onset and/or progression of a neoplasm. More particularly, the present invention is directed to nucleic acid molecules in respect of which changes to the DNA or to the RNA or protein expression profiles are indicative of the onset and/or progression of a large intestine neoplasm, such as an adenoma or an adenocarcinoma. The DNA or the expression profiles of the present invention are useful in a range of applications including, but not limited to, those relating to the diagnosis and/or monitoring of colorectal neoplasms, such as colorectal adenocarcinomas. Accordingly, in a related aspect the present invention is directed to a method of screening a subject for the onset, predisposition to the onset and/or progression of a neoplasm by screening for modulation in the DNA or the RNA or protein expression profile of one or more nucleic acid molecule markers.

### BACKGROUND OF THE INVENTION

Bibliographic details of the publications referred to by author in this specification are collected alphabetically at the end of the description.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

Adenomas are benign tumours, or neoplasms, of epithelial origin which are derived from glandular tissue or exhibit clearly defined glandular structures. Some adenomas show recognisable tissue elements, such as fibrous tissue (fibroadenomas) and epithelial structure, while others, such as bronchial adenomas, produce active compounds that might give rise to clinical syndromes.

Adenomas may progress to become an invasive neoplasm and are then termed adenocarcinomas. Accordingly, adenocarcinomas are defined as malignant epithelial tumours arising from glandular structures, which are constituent parts of many organs of the body. The term adenocarcinoma is also applied to tumours showing a glandular growth pattern. These tumours may be sub-classified according to the substances that they produce, for example mucus secreting and serous adenocarcinomas, or to the microscopic arrangement of their cells into patterns, for example papillary and follicular adenocarcinomas. These carcinomas may be solid or cystic (cystadenocarcinomas). Each organ may produce tumours showing a variety of histological types, for example the ovary may produce both mucinous and cystadenocarcinoma.

Adenomas in different organs behave differently. In general, the overall chance of carcinoma being present within an adenoma (i.e. a focus of cancer having developed within a benign lesion) is approximately 5%. However, this is related to size of an adenoma. For instance, in the large bowel (colon and rectum specifically) occurrence of a cancer within an adenonma is rare in adenomas of less than 1 centimeter. Such a development is estimated at 40 to 50% in adenomas which are greater than 4 centimeters and show certain histopathological change such as villous change, or high grade dysplasia. Adenomas with higher degrees of dysplasia have a higher incidence of carcinoma. In any given colorectal adenoma, the predictors of the presence of cancer now or the future occurrence of cancer in the organ include size (especially greater than 9 mm) degree of change from tubular to villous morphology, presence of high grade dysplasia and the morphological change described as “serrated adenoma”. In any given individual, the additional features of increasing age, familial occurrence of colorectal adenoma or cancer, male gender or multiplicity of adenomas, predict a future increased risk for cancer in the organ—so-called risk factors for cancer. Except for the presence of adenomas and its size, none of these is objectively defined and all those other than number and size are subject to observer error and to confusion as to precise definition of the feature in question. Because such factors can be difficult to assess and define, their value as predictors of current or future risk for cancer is imprecise.

Once a sporadic adenoma has developed, the chance of a new adenoma occurring is approximately 30% within 26 months.

Colorectal adenomas represent a class of adenomas which are exhibiting an increasing incidence, particularly in more affluent countries. The causes of adenoma, and of progression to adenocarcinoma, are still the subject of intensive research. To date it has been speculated that in addition to genetic predisposition, environmental factors (such as diet) play a role in the development of this condition. Most studies indicate that the relevant environmental factors relate to high dietary fat, low fibre, low vegetable intake, smoking, obesity, physical inactivity and high refined carbohydrates.

Colonic adenomas are localised areas of dysplastic epithelium which initially involve just one or several crypts and may not protrude from the surface, but with increased growth in size, usually resulting from an imbalance in proliferation and/or apoptosis, they may protrude. Adenomas can be classified in several ways. One is by their gross appearance and the major descriptors include degrees of protrusion: flat sessile (i.e. protruding but without a distinct stalk) or pedunculated (i.e. having a stalk). Other gross descriptors include actual size in the largest dimension and actual number in the colon/rectum. While small adenomas (less than say or 10 millimetres) exhibit a smooth tan surface, pedunculated and especially larger adenomas tend to have a cobblestone or lobulated red-brown surface. Larger sessile adenomas may exhibit a more delicate villous surface. Another set of descriptors include the histopathological classification; the prime descriptors of clinical value include degree of dysplasia (low or high), whether or not a focus of invasive cancer is present, degree of change from tubular gland formation to villous gland formation (hence classification is tubular, villous or tubulovillous), presence of admixed hyperplastic change and of so-called “serrated” adenomas and its subgroups. Adenomas can be situated at any site in the colon and/or rectum although they tend to be more common in the rectum and distal colon. All of these descriptors, with the exception of number and size, are relatively subjective and subject to interobserver disagreement.

The various descriptive features of adenomas are of value not just to ascertain the neoplastic status of any given adenomas when detected, but also to predict a person's future risk of developing colorectal adenomas or cancer. Those features of an adenoma or number of adenomas in an individual that point to an increased future risk for cancer or recurrence of new adenomas include: size of the largest adenoma (especially 10 mm or larger), degree of villous change (especially at least 25% such change and particularly 100% such change), high grade dysplasia, number (3 or more of any size or histological status) or presence of serrated adenoma features. None except size or number is objective and all are relatively subjective and subject to interobserver disagreement. These predictors of risk for future neoplasia (hence “risk”) are vital in practice because they are used to determine the rate and need for and frequency of future colonoscopic surveillance. More accurate risk classification might thus reduce workload of colonoscopy, make it more cost-effective and reduce the risk of complications from unnecessary procedures.

Adenomas are generally asymptomatic, therefore rendering difficult their diagnosis and treatment at a stage prior to when they might develop invasive characteristics and so became cancer. It is technically impossible to predict the presence or absence of carcinoma based on the gross appearance of adenomas, although larger adenomas are more likely to show a region of malignant change than are smaller adenomas. Sessile adenomas exhibit a higher incidence of malignancy than pedunculated adenomas of the same size. Some adenomas result in blood loss which might be observed or detectable in the stools; while sometimes visible by eye, it is often, when it occurs, microscopic or “occult”. Larger adenomas tend to bleed more than smaller adenomas. However, since blood in the stool, whether overt or occult, can also be indicative of non-adenonmatous conditions, the accurate diagnosis of adenoma is rendered difficult without the application of highly invasive procedures such as colonoscopy combined with tissue acquisition by either removal (i.e. polypectomy) or biopsy and subsequent histopathological analysis.

Accordingly, there is an on-going need to elucidate the causes of adenoma and to develop more informative diagnostic protocols or aids to diagnosis that enable one to direct colonoscopy at people more likely to have adenomas. These adenomas may be high risk, advanced or neither of these, in particular protocols which will enable the rapid, routine and accurate diagnosis of adenoma. Furthermore, it can be difficult after colonoscopy to be certain that all adenomas have been removed, especially in a person who has had multiple adenomas. An accurate screening test may minimise the need to undertake an early second colonoscopy to ensure that the colon has been cleared of neoplasms. Accordingly, the identification of molecular markers for adenomas would provide means for understanding the cause of adenomas and cancer, improving diagnosis of adenomas including development of useful screening tests, elucidating the histological stage of an adenoma, characterising a patient's future risk for colorectal neoplasia on the basis of the molecular state of an adenoma and facilitating treatment of adenomas.

To date, research has focused on the identification of gene mutations which lead to the development of colorectal neoplasms. In work leading up to the present invention, however, it has been determined that changes in the DNA or the RNA or protein expression profiles of genes which are also expressed in healthy individuals are indicative of the development of neoplasms of the large intestine, such as adenomas and adenocarcinomas. It has been further determined that in relation to neoplasms of the large intestine, diagnosis can be made based on screening for one or more of a panel of these differentially expressed genes. In a related aspect, it has still further been determined that to the extent that neoplastic tissue has been identified either by the method of the invention or by some other method, the present invention provides still further means of characterising that tissue as an adenoma or a cancer. In yet another aspect, it has been determined that a proportion of these genes are characterised by gene expression which occurs in the context of non-neoplastic tissue but not in the context of neoplastic tissue, thereby facilitating the development of qualitative analyses which do not require a relative analysis to be performed against a non-neoplastic or normal control reference level. Accordingly, the inventors have identified a panel of genes which facilitate the diagnosis of adenocarcinoma and adenoma development and/or the monitoring of conditions characterised by the development of these types of neoplasms.

### SUMMARY OF THE INVENTION

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

As used herein, the term “derived from” shall be taken to indicate that a particular integer or group of integers has originated from the species specified, but has not necessarily been obtained directly from the specified source. Further, as used herein the singular forms of “a”, “and” and “the” include plural referents unless the context clearly dictates otherwise.

Unless otherwise defined, all technical and scientific terms used herein have the same meaning as commonly understood by one of ordinary skill in the art to which this invention belongs.

The subject specification contains amino acid and nucleotide sequence information prepared using the programme PatentIn Version 3.4, presented herein after the bibliography. Each amino acid and nucleotide sequence is identified in the sequence listing by the numeric indicator <210> followed by the sequence identifier (eg. <210>1, <210>2, etc). The length, type of sequence (amino acid, DNA, etc.) and source organism for each sequence is indicated by information provided in the numeric indicator fields <211>m<212> and <213>, respectively. Amino acid and nucleotide sequences referred to in the specification are identified by the indicator SEQ ID NO: followed by the sequence identifier (eg. SEQ ID NO: 1, SEQ ID NO: 2, etc). The sequence identifier referred to in the specification correlates to the information provided in numeric indicator field <400> in the sequence listing, which is followed by the sequence identifier (eg. <400>1, <400>2, etc). That is SEQ ID NO: 1 as detailed in the specification correlates to the sequence indicated as <400>1 in the sequence listing.

One aspect of the present invention is directed to a method of screening for the onset or predisposition to the onset of a large intestine neoplasm in an individual, said method comprising assessing the level of expression of one or more genes or transcripts selected from:

the gene, genes or transcripts detected by Affymetrix probeset IDs:

| | | | |
|---|---|---|---|
| (i) | 200600_at | 210133_at | 227235_at |
| | 200621_at | 210139_s_at | 227265_at |
| | 200795_at | 210298_x_at | 227404_s_at |
| | 200799_at | 210299_s_at | 227529_s_at |
| | 200845_s_at | 210302_s_at | 227561_at |
| | 200859_x_at | 210495_x_at | 227623_at |
| | 200897_s_at | 210517_s_at | 227662_at |
| | 200974_at | 210764_s_at | 227705_at |
| | 200986_at | 210809_s_at | 227727_at |
| | 201041_s_at | 210946_at | 227826_s_at |
| | 201058_s_at | 210982_s_at | 227827_at |
| | 201061_s_at | 211161_s_at | 228202_at |
| | 201069_at | 211548_s_at | 228504_at |
| | 201105_at | 211596_s_at | 228507_at |
| | 201137_s_at | 211643_x_at | 228640_at |
| | 201141_at | 211644_x_at | 228706_s_at |
| | 201150_s_at | 211645_x_at | 228707_at |
| | 201289_at | 211671_s_at | 228750_at |
| | 201300_s_at | 211696_x_at | 228766_at |
| | 201324_at | 211719_x_at | 228846_at |
| | 201348_at | 211798_x_at | 228854_at |
| | 201426_s_at | 211813_x_at | 228885_at |
| | 201427_s_at | 211848_s_at | 229530_at |
| | 201438_at | 211889_x_at | 229839_at |
| | 201496_x_at | 211896_s_at | 230087_at |
| | 201497_x_at | 211959_at | 230264_s_at |
| | 201539_s_at | 211964_at | 230788_at |
| | 201540_at | 211985_s_at | 230830_at |
| | 201616_s_at | 211990_at | 231120_x_at |
| | 201617_x_at | 211991_s_at | 231579_s_at |
| | 201645_at | 212077_at | 231773_at |
| | 201667_at | 212091_s_at | 234764_x_at |
| | 201739_at | 212097_at | 234987_at |
| | 201743_at | 212136_at | 236300_at |
| | 201744_s_at | 212158_at | 236313_at |
| | 201842_s_at | 212185_x_at | 242317_at |
| | 201852_x_at | 212192_at | 200884_at |
| | 201858_s_at | 212195_at | 201495_x_at |
| | 201859_at | 212230_at | 202266_at |
| | 201865_x_at | 212233_at | 202350_s_at |
| | 201893_x_at | 212265_at | 202731_at |
| | 201920_at | 212288_at | 202741_at |
| | 201957_at | 212386_at | 202742_s_at |
| | 202007_at | 212387_at | 202768_at |
| | 202037_s_at | 212397_at | 202838_at |
| | 202069_s_at | 212414_s_at | 203058_s_at |
| | 202133_at | 212419_at | 203060_s_at |
| | 202222_s_at | 212464_s_at | 203240_at |
| | 202242_at | 212667_at | 203296_s_at |
| | 202274_at | 212671_s_at | 203343_at |
| | 202283_at | 212713_at | 203474_at |
| | 202291_s_at | 212730_at | 203638_s_at |
| | 202388_at | 212764_at | 203963_at |
| | 202555_s_at | 212859_x_at | 204018_x_at |
| | 202620_s_at | 212956_at | 204034_at |
| | 202686_s_at | 213068_at | 204036_at |
| | 202746_at | 213071_at | 204130_at |
| | 202760_s_at | 213428_s_at | 204388_s_at |
| | 202766_s_at | 213509_x_at | 204389_at |
| | 202888_s_at | 213624_at | 204508_s_at |
| | 202920_at | 213746_s_at | 204532_x_at |
| | 202953_at | 213891_s_at | 204607_at |
| | 202957_at | 214027_x_at | 204673_at |
| | 202992_at | 214038_at | 204818_at |
| | 202994_s_at | 214091_s_at | 204895_x_at |
| | 202995_s_at | 214142_at | 204897_at |
| | 203000_at | 214414_x_at | 205112_at |
| | 203001_s_at | 214505_s_at | 205259_at |
| | 203066_at | 214677_x_at | 205403_at |
| | 203131_at | 214696_at | 205480_s_at |
| | 203305_at | 214752_x_at | 205554_s_at |
| | 203382_s_at | 214768_x_at | 205593_s_at |
| | 203477_at | 214777_at | 205892_s_at |
| | 203645_s_at | 215049_x_at | 205929_at |
| | 203680_at | 215076_s_at | 206000_at |
| | 203729_at | 215118_s_at | 206094_x_at |
| | 203748_x_at | 215176_x_at | 206262_at |
| | 203766_s_at | 215193_x_at | 206377_at |
| | 203881_s_at | 215382_x_at | 206385_s_at |
| | 203908_at | 215388_s_at | 206664_at |
| | 203913_s_at | 215657_at | 207126_x_at |
| | 203914_x_at | 216207_x_at | 207245_at |
| | 203951_at | 216401_x_at | 207390_s_at |
| | 203980_at | 216442_x_at | 207392_x_at |
| | 204069_at | 216474_x_at | 207432_at |
| | 204083_s_at | 216576_x_at | 207761_s_at |
| | 204122_at | 216834_at | 208596_s_at |
| | 204135_at | 216984_x_at | 208920_at |
| | 204326_x_at | 217148_x_at | 209114_at |
| | 204438_at | 217179_x_at | 209374_s_at |
| | 204457_s_at | 217235_x_at | 209458_x_at |
| | 204570_at | 217258_x_at | 209791_at |
| | 204688_at | 217378_x_at | 210107_at |
| | 204697_s_at | 217480_x_at | 210524_x_at |
| | 204719_at | 217546_at | 210735_s_at |
| | 204745_x_at | 217757_at | 211372_s_at |
| | 204834_at | 217762_s_at | 211538_s_at |
| | 204894_s_at | 217764_s_at | 211549_s_at |
| | 204931_at | 217767_at | 211637_x_at |
| | 204938_s_at | 217897_at | 211699_x_at |
| | 204939_s_at | 217967_s_at | 211745_x_at |
| | 204940_at | 218087_s_at | 212224_at |
| | 204955_at | 218162_at | 212592_at |
| | 205097_at | 218224_at | 212741_at |
| | 205200_at | 218312_s_at | 212814_at |
| | 205267_at | 218353_at | 213317_at |
| | 205382_s_at | 218418_s_at | 213451_x_at |
| | 205412_at | 218468_s_at | 213629_x_at |
| | 205433_at | 218469_at | 213921_at |
| | 205464_at | 218559_s_at | 213953_at |
| | 205547_s_at | 218756_s_at | 214164_x_at |
| | 205683_x_at | 219014_at | 214433_s_at |
| | 205935_at | 219087_at | 214598_at |
| | 205950_s_at | 219508_at | 214916_x_at |
| | 206134_at | 219607_s_at | 215125_s_at |
| | 206143_at | 219669_at | 215299_x_at |
| | 206149_at | 219799_s_at | 215867_x_at |
| | 206198_s_at | 220026_at | 216336_x_at |
| | 206199_at | 220037_s_at | 216491_x_at |
| | 206208_at | 220376_at | 216510_x_at |
| | 206209_s_at | 220834_at | 217022_s_at |
| | 206422_at | 221541_at | 217109_at |
| | 206461_x_at | 221667_s_at | 217110_s_at |
| | 206561_s_at | 221747_at | 217165_x_at |
| | 206576_s_at | 221748_s_at | 217232_x_at |
| | 206637_at | 222043_at | 217414_x_at |
| | 206641_at | 222162_s_at | 218541_s_at |
| | 206710_s_at | 222453_at | 218546_at |
| | 206784_at | 222513_s_at | 219059_s_at |
| | 207003_at | 222717_at | 219543_at |
| | 207080_s_at | 222722_at | 219796_s_at |
| | 207134_x_at | 223121_s_at | 219948_x_at |
| | 207266_x_at | 223122_s_at | 220075_s_at |
| | 207502_at | 223235_s_at | 220266_s_at |
| | 207961_x_at | 223343_at | 220468_at |
| | 207977_s_at | 223395_at | 220645_at |
| | 207980_s_at | 223551_at | 220812_s_at |
| | 208131_s_at | 223623_at | 221004_s_at |
| | 208370_s_at | 223952_x_at | 221305_s_at |
| | 208383_s_at | 224009_x_at | 221584_s_at |
| | 208399_s_at | 224352_s_at | 221841_s_at |
| | 208450_at | 224412_s_at | 221896_s_at |
| | 208581_x_at | 224480_s_at | 223484_at |
| | 208747_s_at | 224560_at | 223597_at |
| | 208763_s_at | 224663_s_at | 223754_at |
| | 208788_at | 224694_at | 224342_x_at |
| | 208789_at | 224823_at | 224989_at |
| | 208791_at | 224836_at | 224990_at |
| | 208792_s_at | 224840_at | 225458_at |
| | 208894_at | 224959_at | 225728_at |
| | 209047_at | 224963_at | 226147_s_at |
| | 209074_s_at | 224964_s_at | 226302_at |
| | 209101_at | 225207_at | 226594_at |
| | 209116_x_at | 225242_s_at | 226654_at |
| | 209138_x_at | 225269_s_at | 226811_at |
| | 209147_s_at | 225275_at | 227052_at |
| | 209156_s_at | 225353_s_at | 227522_at |
| | 209167_at | 225381_at | 227682_at |
| | 209170_s_at | 225442_at | 227725_at |
| | 209191_at | 225575_at | 227735_s_at |
| | 209209_s_at | 225602_at | 227736_at |
| | 209210_s_at | 225604_s_at | 228133_s_at |
| | 209283_at | 225626_at | 228195_at |
| | 209301_at | 225688_s_at | 228232_s_at |
| | 209312_x_at | 225710_at | 228241_at |
| | 209335_at | 225720_at | 228469_at |
| | 209357_at | 225721_at | 228961_at |
| | 209373_at | 225782_at | 229070_at |
| | 209436_at | 225894_at | 229254_at |
| | 209457_at | 225895_at | 229659_s_at |
| | 209496_at | 226001_at | 229831_at |
| | 209498_at | 226051_at | 230595_at |
| | 209612_s_at | 226084_at | 231925_at |
| | 209613_s_at | 226103_at | 231975_s_at |
| | 209621_s_at | 226303_at | 233565_s_at |
| | 209651_at | 226304_at | 235146_at |
| | 209656_s_at | 226333_at | 235766_x_at |
| | 209667_at | 226430_at | 235849_at |
| | 209668_x_at | 226492_at | 238143_at |
| | 209687_at | 226682_at | 238750_at |
| | 209735_at | 226694_at | 238751_at |
| | 209763_at | 226818_at | 239272_at |
| | 209868_s_at | 226834_at | 241994_at |
| | 209948_at | 226841_at | 242447_at |
| | 210084_x_at | 227006_at | 242601_at |
| | 227099_s_at | 227061_at | 243278_at; and/or |
| (ii) | CLCA4 | SGK | MT1X |
| | ZG16 | CFL2 | AOC3 |
| | CA2 | C1S | PPAP2A |
| | CA1 | SELENBP1 | ZSCAN18 |
| | MS4A12 | MT1E | IVD |
| | AQP8 | ADAMTS1 | SFRP1 |
| | SLC4A4 | ITM2A | COL4A2 |
| | CEACAM7 | POU2AF1 | GPM6B |
| | TAGLN | FAM55D | EPB41L3 |
| | GUCA1B | C6orf204 | MAOA |
| | GCG | AKAP12 | DMD |
| | ADH1B | TUBB6 | MSRB3 |
| | UGT2B17 | LGALS2 | PLOD2 |
| | ADAMDEC1 | KIAA0828 | C9orf19 |
| | MT1M | MGC14376 | MIER3 |
| | AKR1B10 | PPP1R14A | XDH |
| | FN1 | MUC4 | CLDN23 |
| | MGP | PKIB | SGCE |
| | CXCL12 | PIGR | FOXF2 |
| | PDK4 | ASPN | AGR3 |
| | CA4 | A2M | IGLJ3 |
| | PYY | LOC25845 | QKI |
| | IGHA1 | LGALS1 | LOC399959 |
| | TPM2 | BCHE | ANKRD25 |
| | C6orf105 | ST6GALNAC1 | CRISPLD2 |
| | HPGD | GJA1 | ANK2 |
| | ADH1C | SCNN1B | LOC283666 |
| | CLCA1 | FABP4 | CRYAB |
| | FABP1 | F13A1 | ACAT1 |
| | ENAM | CD36 | IGL@ |
| | CFD | SPARCL1 | PBLD |
| | GUCA2B | ZCWPW2 | CCL8 |
| | FBLN1 | TNC | LIFR |
| | LOC63928 | MT1A | HLA-DRB1 |
| | ABCA8 | LOC652745 | UGP2 |
| | POSTN | MALL | IGKV1D-13 |
| | DCN | GNG2 | AP1S2 |
| | ITLN1 | DNASE1L3 | EMP3 |
| | COL6A2 | EGR1 | MMP28 |
| | FCGBP | CMBL | UGT2A3 |
| | SLC26A2 | GCNT3 | RGS5 |
| | PGM5 | SERPING1 | PTGIS |
| | DMN | MEIS1 | DUSP5 |
| | GPNMB | EDN3 | MFAP4 |
| | IGFBP5 | MSN | UGT1A6 |
| | CLEC3B | MT1G | PRKAR2B |
| | LOC253012 | TPSAB1 | HHLA2 |
| | DPT | GPX3 | LOC652128 |
| | PCK1 | CDKN2B | C3 |
| | CNN1 | FOSB | ATP2B4 |
| | HSD17B2 | HSPA1A | HBA1 |
| | PLAC8 | CYBRD1 | TCF21 |
| | TMEM47 | PTGER4 | PPID |
| | OGN | MAG1 | PPAP2B |
| | CALD1 | BEST2 | SPON1 |
| | ACTG2 | HLA-DQA1 | PHLDB2 |
| | MGC4172 | PRIMA1 | RARRES2 |
| | MAB21L2 | MT1F | ETHE1 |
| | RPL24 | MAFB | MMP2 |
| | ABCG2 | FAM107A | SRI |
| | CCDC80 | PRKACB | CNTN3 |
| | UGT1A1 | SELM | RGS2 |
| | MRC1 | TYROBP | COL6A1 |
| | HSD11B2 | TNS1 | FBN1 |
| | ANPEP | MYH11 | MXD1 |
| | MATN2 | ITM2C | PLCE1 |
| | PRNP | CES2 | KCNMB1 |
| | ABI3BP | MS4A4A | CALM1 |
| | HLA-C | PDGFRA | HLA-DPB1 |
| | NDE1 | CA12 | SMOC2 |
| | SRPX | FKBP5 | LOC285382 |
| | WWTR1 | HSPB8 | CLIC5 |
| | HMGCS2 | TPSB2 | APOE |
| | LOC646627 | FGL2 | SERPINF1 |
| | KRT20 | C1QB | PPP1R12B |
| | KLF4 | ANGPTL1 | HSPB6 |
| | FHL1 | MEP1A | FNBP1 |
| | ARL14 | GUCY1A3 | C4orf34 |
| | LUM | UGDH | SORBS2 |
| | SORBS1 | DUSP1 | GPA33 |
| | METTL7A | C2orf40 | GALNAC4S-6ST |
| | FAM129A | PLN | CFHR1 |
| | SCARA5 | UGT2B15 | MGC13057 |
| | SI | PDLIM3 | C10orf56 |
| | ACTA2 | TP53INP2 | SULT1A1 |
| | CD177 | ATP8B1 | TTRAP |
| | C10orf99 | ANK3 | CCL28 |
| | COL15A1 | CTGF | IDH3A |
| | NR3C2 | MUCDHL | EDG2 |
| | DHRS9 | SDPR | UGT1A8 |
| | LMOD1 | COL14A1 | RAB27A |
| | EFEMP1 | DSCR1 | ANTXR1 |
| | GREM1 | CITED2 | EMP1 |
| | IL1R2 | MT1H | CSRP1 |
| | LOC387763 | NEXN | PLEKHC1 |
| | TIMP3 | MUC2 | LOC572558 |
| | MYLK | NID1 | FOXP2 |
| | CLDN8 | HBB | HSPA2 |
| | RDX | GCNT2 | ATP1A2 |
| | TSPAN7 | C20orf118 | TNXB |
| | TNFRSF17 | SLC20A1 | FUCA1 |
| | SYNPO2 | CD14 | MRGPRF |
| | VIM | KCTD12 | HIGD1A |
| | SMPDL3A | RBMS1 | MFSD4 |
| | P2RY14 | PTRF | AXL |
| | CHGA | TSPAN1 | AQP1 |
| | C15orf48 | UGT1A9 | MAP1B |
| | COL3A1 | COX7A1 | PALLD |
| | CYR61 | MUC12 | MPEG1 |
| | TRPM6 | PDCD4 | KLHL5 |
| | OSTbeta | CAV1 | TCEAL7 |
| | IGLV1-44 | FAM46C | FILIP1L |
| | VSIG2 | LRIG1 | IQGAP2 |
| | IGHM | HLA-DPA1 | PRDX6 |
| | LRRC19 | C1orf115 | RAB31 |
| | CD163 | HBA2 | LOC96610 |
| | CEACAM1 | EDIL3 | FGFR2 |
| | TIMP2 | DES | PAPSS2 |
| | ENTPD5 | MT2A | XLKD1 |
| | DDR2 | KCNMA1 | SMTN |
| | CHRDL1 | GAS1 | C8orf4 |
| | SRGN | TBC1D9 | SDCBP2 |
| | PDE9A | C7 | CCL11 |
| | PMP22 | P2RY1 | ELOVL5 |
| | FLNA | NR3C1 | FOXF1 |
| | STMN2 | STOM | RELL1 |
| | MYL9 | CKB | PNMA1 |
| | SEMA6D | CLU | LOC339562 |
| | PADI2 | SLC26A3 | PALM2-AKAP2 |
| | SEPPI | SDC2 | PAG1 |
| | TGFB1I1 | SST | HCLS1 |
| | SFRP2 | HLA-DRA | RGS1 |
| | UGT1A3 | TSC22D3 | FXYD6 |
| | MS4A7 | IL6ST | OLFML3 |
| | ALDH1A1 | C1QC | COL6A3 |

in a biological sample from said individual wherein a lower level of expression of the genes or transcripts of group (i) and/or group (ii) relative to control levels is indicative of a neoplastic large intestine cell or a cell predisposed to the onset of a neoplastic state.

Another aspect of the present invention provides a method of screening for the onset or predisposition to the onset of a large intestine neoplasm in an individual, said method comprising assessing the level of expression of one or more genes or transcripts selected from:
(i) the gene, genes or transcripts detected by Affymetrix probeset IDs: 220026 at; and/or
(ii) CLCA4

in a biological sample from said individual wherein a lower level of expression of the gene or transcripts of group (i) and/or group (ii) relative to control levels is indicative of a neoplastic large intestine cell or a cell predisposed to the onset of a neoplastic state.

In yet another aspect there is provided a method of screening for the onset or predisposition to the onset of a large intestine neoplasm in an individual, said method comprising assessing the level of expression of one or more genes or transcripts selected from:
(i) the gene, genes or transcripts detected by Affymetrix probeset IDs: 214142 at; and/or
(11) ZG16

in a biological sample from said individual wherein a lower level of expression of the genes or transcripts of group (i) and/or group (ii) relative to control levels is indicative of a neoplastic large intestine cell or a cell predisposed to the onset of a neoplastic state.

In still another aspect there is provided a method of screening for the onset or predisposition to the onset of a large intestine neoplasm in an individual, said method comprising assessing the level of expression of one or more genes or transcripts selected from:
(i) the gene, genes or transcripts detected by Affymetrix probeset IDs: 209301_at 205950_s_at; and/or
(ii) CA2 CA1

in a biological sample from said individual wherein a lower level of expression of the genes or transcripts of group (i) and/or group (ii) relative to control levels is indicative of a neoplastic large intestine cell or a cell predisposed to the onset of a neoplastic state.

In still yet another aspect there is provided a method of screening for the onset or predisposition to the onset of a large intestine neoplasm in an individual, said method comprising assessing the level of expression of one or more genes or transcripts selected from:
(i) the gene, genes or transcripts detected by Affymetrix probeset IDs: 220834 at; and/or
(ii) MS4A12

in a biological sample from said individual wherein a lower level of expression of the genes or transcripts of group (i) and/or group (ii) relative to control levels is indicative of a neoplastic large intestine cell or a cell predisposed to the onset of a neoplastic state.

In yet still another aspect there is provided a method of screening for the onset or predisposition to the onset of a large intestine neoplasm in an individual, said method comprising assessing the level of expression of one or more genes or transcripts selected from:
(i) the gene, genes or transcripts detected by Affymetrix probeset IDs: 206784 at; and/or
(ii) AQP8

in a biological sample from said individual wherein a lower level of expression of the genes or transcripts of group (i) and/or group (ii) relative to control levels is indicative of a neoplastic large intestine cell or a cell predisposed to the onset of a neoplastic state.

In a further aspect there is provided a method of screening for the onset or predisposition to the onset of a large intestine neoplasm in an individual, said method comprising assessing the level of expression of one or more genes or transcripts selected from:
the gene, genes or transcripts detected by Affymetrix probeset IDs: 203908_at, 206198_s_at, 205547_s_at, 207003_at, 206422_at, 209613_s_at, 207245_at; and/or
(ii) SLC4A4, CEACAM7, TAGLN, GUCA1B, GCG, ADH1B, UGT2B17,

in a biological sample from said individual wherein a lower level of expression of the genes or transcripts of group (i) and/or group (ii) relative to control levels is indicative of a neoplastic large intestine cell or a cell predisposed to the onset of a neoplastic state.

In another further aspect there is provided a method of screening for the onset or predisposition to the onset of a large intestine neoplasm in an individual, said method comprising assessing the level of expression of one or more genes or transcripts selected from:
the gene, genes or transcripts detected by Affymetrix probeset IDs: 203908_at, 206198_s_at, 205547_s_at, 207003_at, 206422_at, 209613_s_at, 207245_at; and/or
(ii) SLC4A4, CEACAM7, TAGLN, GUCA1B, GCG, ADH1B, UGT2B17,

in a biological sample from said individual wherein a lower level of expression of the genes or transcripts of group (i) and/or group (ii) relative to control levels is indicative of a neoplastic large intestine cell or a cell predisposed to the onset of a neoplastic state.

In still another further aspect there is provided a method of screening for the onset or predisposition to the onset of a large intestine neoplasm in an individual, said method comprising screening the level of expression of one or more genes or transcripts selected from:

the gene, genes or transcripts detected by Affymetrix probeset IDs:

| | | | |
|---|---|---|---|
| (i) | 225207_at | 211548_s_at | 205382_s_at |
| | 206208_at | 206262_at | 207502_at |
| | 207080_s_at | 210107_at | 202995_s_at |
| | 215118_s_at | 205892_s_at | 206149_at |
| | 204083_s_at | 212592_at | 204719_at |
| | 229070_at; and/or | | |
| (ii) | PDK4 | HPGD | CFD |
| | CA4 | ADH1C | GUCA2B |
| | PYY | CLCA1 | FBLN1 |
| | IGHA1 | FABP1 | LOC63928 |
| | TPM2 | ENAM | ABCA8 |
| | C6orf105 | | |

in a biological sample from said individual wherein a lower level of expression of the genes or transcripts of group (i) and/or group (ii) relative to control levels is indicative of a neoplastic large intestine cell or a cell predisposed to the onset of a neoplastic state.

In yet still yet another further aspect there is provided a method of screening for the onset or predisposition to the onset of a large intestine neoplasm in an individual, said method comprising screening the level of expression of one or more genes or transcripts selected from:

the gene, genes or transcripts detected by Affymetrix probeset IDs:

| | | | |
|---|---|---|---|
| (i) | 210809_s_at | 201617_x_at | 202133_at |
| | 201893_x_at | 202274_at | 204607_at |
| | 223597_at | 218756_s_at | 238143_at |
| | 209156_s_at | 210302_s_at | 213953_at |
| | 203240_at | 228885_at | 220266_s_at |
| | 224963_at | 209735_at | 210299_s_at |
| | 226303_at | 228504_at | 220468_at |
| | 212730_at | 225242_s_at | 201744_s_at |
| | 201141_at | 215125_s_at | 218087_s_at |
| | 211959_at | 204438_at | 207761_s_at |
| | 205200_at | 204130_at | 217967_s_at |
| | 242601_at | 202888_s_at | 229839_at |
| | 213068_at | 202350_s_at | 206664_at |
| | 208383_s_at | 201300_s_at | 200974_at |
| | 203951_at | 223395_at | 219669_at |
| | 204818_at | 214768_x_at | 227736_at |
| | 219014_at | 228133_s_at | 203477_at |
| | 209656_s_at | 204955_at | 205259_at |
| | 222722_at; and/or | | |
| (ii) | POSTN | OGN | WWTR1 |
| | DCN | CALD1 | HMGCS2 |
| | ITLN1 | ACTG2 | LOC646627 |
| | COL6A2 | MGC4172 | KRT20 |
| | FCGBP | MAB21L2 | KLF4 |
| | SLC26A2 | RPL24 | FHL1 |
| | PGM5 | ABCG2 | ARL14 |
| | DMN | CCDC80 | LUM |
| | GPNMB | UGT1A1 | SORBS1 |
| | IGFBP5 | MRC1 | METTL7A |
| | CLEC3B | HSD11B2 | FAM129A |
| | LOC253012 | ANPEP | SCARA5 |
| | DPT | MATN2 | SI |
| | PCK1 | PRNP | ACTA2 |
| | CNN1 | ABI3BP | CD177 |
| | HSD17B2 | HLA-C | C10orf99 |
| | PLAC8 | NDE1 | COL15A1 |
| | TMEM47 | SRPX | NR3C2 |

in a biological sample from said individual wherein a lower level of expression of the genes or transcripts of group (i) and/or group (ii) relative to control levels is indicative of a neoplastic large intestine cell or a cell predisposed to the onset of a neoplastic state.

In a related aspect the present invention is directed to a method of screening for the onset or predisposition to the onset of a large intestine neoplasm in an individual, said method comprising screening the level of expression of one or more genes or transcripts selected from:

the gene, genes or transcripts detected by Affymetrix probeset IDs:

| | | | |
|---|---|---|---|
| (i) | 200600_at | 208788_at | 215382_x_at |
| | 200665_s_at | 208789_at | 215388_s_at |
| | 200799_at | 208894_at | 216442_x_at |
| | 200845_s_at | 209047_at | 216474_x_at |
| | 200859_x_at | 209101_at | 216834_at |
| | 200897_s_at | 209138_x_at | 217480_x_at |
| | 200974_at | 209147_s_at | 217757_at |
| | 200986_at | 209156_s_at | 217762_s_at |
| | 201041_s_at | 209191_at | 217764_s_at |
| | 201061_s_at | 209209_s_at | 217767_at |
| | 201069_at | 209210_s_at | 217897_at |
| | 201105_at | 209312_x_at | 218162_at |
| | 201137_s_at | 209335_at | 218224_at |
| | 201141_at | 209436_at | 218312_s_at |
| | 201150_s_at | 209457_at | 218353_at |
| | 201289_at | 209496_at | 218418_s_at |
| | 201300_s_at | 209621_s_at | 218468_s_at |
| | 201426_s_at | 209651_at | 218469_at |
| | 201438_at | 209656_s_at | 218559_s_at |
| | 201616_s_at | 209868_s_at | 219087_at |
| | 201617_x_at | 210084_x_at | 219607_s_at |
| | 201645_at | 210133_at | 221541_at |
| | 201667_at | 210139_s_at | 222043_at |
| | 201743_at | 210495_x_at | 222453_at |
| | 201744_s_at | 210517_s_at | 222513_s_at |
| | 201842_s_at | 210764_s_at | 223121_s_at |
| | 201852_x_at | 210809_s_at | 223122_s_at |
| | 201858_s_at | 210982_s_at | 223235_s_at |
| | 201859_at | 211161_s_at | 223343_at |
| | 201865_x_at | 211596_s_at | 224560_at |
| | 201893_x_at | 211671_s_at | 224694_at |
| | 201920_at | 211719_x_at | 224840_at |
| | 202007_at | 211813_x_at | 224964_s_at |
| | 202069_s_at | 211896_s_at | 225242_s_at |
| | 202133_at | 211959_at | 225269_s_at |
| | 202283_at | 211964_at | 225353_s_at |
| | 202291_s_at | 211985_s_at | 225381_at |
| | 202403_s_at | 211990_at | 225442_at |
| | 202620_s_at | 211991_s_at | 225602_at |
| | 202686_s_at | 212077_at | 225604_s_at |
| | 202760_s_at | 212091_s_at | 225626_at |
| | 202766_s_at | 212136_at | 225688_s_at |
| | 202953_at | 212158_at | 225710_at |
| | 202957_at | 212185_x_at | 226001_at |
| | 202994_s_at | 212195_at | 226051_at |
| | 202995_s_at | 212230_at | 226084_at |
| | 203066_at | 212233_at | 226103_at |
| | 203131_at | 212265_at | 226430_at |
| | 203305_at | 212386_at | 226682_at |
| | 203382_s_at | 212387_at | 226694_at |
| | 203477_at | 212397_at | 226818_at |
| | 203645_s_at | 212414_s_at | 226834_at |
| | 203680_at | 212419_at | 226841_at |
| | 203729_at | 212464_s_at | 227061_at |
| | 203748_x_at | 212667_at | 227099_s_at |
| | 204069_at | 212671_s_at | 227235_at |
| | 204122_at | 212713_at | 227404_s_at |
| | 204135_at | 212764_at | 227529_s_at |
| | 204438_at | 212956_at | 227561_at |
| | 204457_s_at | 213428_s_at | 227623_at |
| | 204570_at | 213509_x_at | 227705_at |
| | 204688_at | 213746_s_at | 227727_at |
| | 205412_at | 213891_s_at | 228507_at |
| | 205683_x_at | 214038_at | 228750_at |
| | 205935_at | 214677_x_at | 228846_at |
| | 207134_x_at | 214752_x_at | 229530_at |
| | 207266_x_at | 215049_x_at | 230264_s_at |
| | 208131_s_at | 215076_s_at | 231579_s_at |
| | 208370_s_at | 215193_x_at | 234987_at; and/or |
| | 208747_s_at | | |
| (ii) | A2M | FBN1 | PALLD |
| | ACAT1 | FILIP1L | PALM2-AKAP2 |
| | ACTA2 | FKBP5 | PDGFRA |
| | AKAP12 | FLNA | PDLIM3 |
| | ANKRD25 | FN1 | PHLDB2 |
| | ANTXR1 | FOXF1 | PLEKHC1 |
| | AP1S2 | FXYD6 | PLOD2 |
| | APOE | GALNAC4S-6ST | PMP22 |
| | AQP1 | GAS1 | PNMA1 |
| | ASPN | GJA1 | POSTN |
| | ATP2B4 | GNG2 | PPAP2A |
| | AXL | GPNMB | PPAP2B |
| | C10orf56 | GREM1 | PRDX6 |
| | C1QB | GUCY1A3 | PRKAR2B |
| | C1QC | HCLS1 | PRNP |
| | C1S | HLA-DPA1 | PTGIS |
| | C20orf118 | HLA-DPB1 | PTRF |
| | C3 | HLA-DQA1 | QKI |
| | C9orf19 | HLA-DRA | RAB31 |
| | CALD1 | HLA-DRB1 | RARRES2 |
| | CALM1 | HSPA1A | RBMS1 |
| | CCDC80 | IDH3A | RDX |
| | CCL11 | IGFBP5 | RELL1 |
| | CCL8 | IGL@ | RGS1 |
| | CD14 | IGLJ3 | RGS5 |
| | CD163 | IL6ST | SDC2 |
| | CES2 | KLHL5 | SELM |
| | CFHR1 | LGALS1 | SEPT6 |
| | CLU | LOC283666 | SERPINF1 |
| | COL14A1 | LOC339562 | SERPING1 |
| | COL15A1 | LOC387763 | SFRP2 |
| | COL1A2 | LOC399959 | SGCE |
| | COL3A1 | LRIG1 | SLC20A1 |
| | COL4A2 | LUM | SMOC2 |
| | COL6A1 | MAFB | SORBS1 |
| | COL6A2 | MAP1B | SPARC |
| | COL6A3 | MEIS1 | SPON1 |
| | COX7A1 | MFAP4 | SRGN |
| | CRISPLD2 | MGP | STOM |
| | CTGF | MMP2 | TBC1D9 |
| | CYBRD1 | MPEG1 | TCEAL7 |
| | CYR61 | MRC1 | TGFB1I1 |
| | DCN | MRGPRF | TIMP2 |
| | DDR2 | MS4A4A | TIMP3 |
| | DSCR1 | MS4A7 | TMEM47 |
| | DUSP1 | MSN | TNC |
| | DUSP5 | MT2A | TPSAB1 |
| | EFEMP1 | MXD1 | TPSB2 |
| | EGR1 | NEXN | TUBB6 |
| | ELOVL5 | NID1 | TYROBP |
| | EMP3 | NR3C1 | VIM |
| | F13A1 | OLFML3 | WWTR1 |
| | FBLN1 | PAG1 | ZSCAN18 |

in a biological sample from said individual wherein a lower level of expression of the genes or transcripts of group (i) and/or group (ii) relative to control levels is indicative of an adenoma cell or a cell predisposed to the onset of an adenoma state.

In another aspect of the present invention there is provided a method of screening for the onset or predisposition to the onset of a large intestine neoplasm in an individual, said method comprising screening the level of expression of one or more genes or transcripts selected from:

the gene or genes detected by Affymetrix probeset IDs:

| | | | |
|---|---|---|---|
| (i) | 200884_at | 208596_s_at | 220812_s_at |
| | 201495_x_at | 208920_at | 221004_s_at |
| | 202266_at | 209114_at | 221305_s_at |
| | 202350_s_at | 209374_s_at | 221584_s_at |
| | 202731_at | 209458_x_at | 221841_s_at |
| | 202741_at | 209791_at | 221896_s_at |
| | 202742_s_at | 210107_at | 223484_at |
| | 202768_at | 210524_x_at | 223597_at |
| | 202838_at | 210735_s_at | 223754_at |
| | 203058_s_at | 211372_s_at | 224342_x_at |
| | 203060_s_at | 211538_s_at | 224989_at |
| | 203240_at | 211549_s_at | 224990_at |
| | 203296_s_at | 211637_x_at | 225458_at |
| | 203343_at | 211699_x_at | 225728_at |
| | 203474_at | 211745_x_at | 226147_s_at |
| | 203638_s_at | 212224_at | 226302_at |
| | 203963_at | 212592_at | 226594_at |
| | 204018_x_at | 212741_at | 226654_at |
| | 204034_at | 212814_at | 226811_at |
| | 204036_at | 213317_at | 227052_at |
| | 204130_at | 213451_x_at | 227522_at |
| | 204388_s_at | 213629_x_at | 227682_at |
| | 204389_at | 213921_at | 227725_at |
| | 204508_s_at | 213953_at | 227735_s_at |
| | 204532_x_at | 214164_x_at | 227736_at |
| | 204607_at | 214433_s_at | 228133_s_at |
| | 204673_at | 214598_at | 228195_at |
| | 204818_at | 214916_x_at | 228232_s_at |
| | 204895_x_at | 215125_s_at | 228241_at |
| | 204897_at | 215299_x_at | 228469_at |
| | 205112_at | 215867_x_at | 228961_at |
| | 205259_at | 216336_x_at | 229070_at |
| | 205403_at | 216491_x_at | 229254_at |
| | 205480_s_at | 216510_x_at | 229659_s_at |
| | 205554_s_at | 217022_s_at | 229831_at |
| | 205593_s_at | 217109_at | 230595_at |
| | 205892_s_at | 217110_s_at | 231925_at |
| | 205929_at | 217165_x_at | 231975_s_at |
| | 206000_at | 217232_x_at | 233565_s_at |
| | 206094_x_at | 217414_x_at | 235146_at |
| | 206262_at | 218541_s_at | 235766_x_at |
| | 206377_at | 218546_at | 235849_at |
| | 206385_s_at | 219059_s_at | 238143_at |
| | 206664_at | 219543_at | 238750_at |
| | 207126_x_at | 219796_s_at | 238751_at |
| | 207245_at | 219948_x_at | 239272_at |
| | 207390_s_at | 220075_s_at | 241994_at |
| | 207392_x_at | 220266_s_at | 242447_at |
| | 207432_at | 220468_at | 242601_at |
| | 207761_s_at | 220645_at | 243278_at; and/or |
| (ii) | ADH1C | HIGD1A | NR3C2 |
| | AGR3 | HMGCS2 | P2RY1 |
| | ALDH1A1 | HPGD | PADI2 |
| | ANK3 | HSD11B2 | PAPSS2 |
| | ARL14 | HSD17B2 | PBLD |
| | ATP1A2 | HSPA2 | PDCD4 |
| | ATP8B1 | IGHA1 | PDE9A |
| | BEST2 | IGHM | PIGR |
| | C10orf99 | IL1R2 | PLCE1 |
| | C15orf48 | IL8 | PPID |
| | C1orf115 | IQGAP2 | PRKACB |
| | C4orf34 | ITLN1 | PTGER4 |
| | C6orf105 | ITM2C | RAB27A |
| | C8orf4 | KCNMA1 | SCARA5 |
| | CA12 | KIAA0828 | SDCBP2 |
| | CCL28 | KLF4 | SELENBP1 |
| | CKB | KRT20 | SI |
| | CLCA1 | LOC253012 | SMTN |
| | CLDN8 | LOC25845 | SORBS2 |
| | CLIC5 | LOC285382 | SRI |
| | CMBL | LOC572558 | SST |
| | CNTN3 | LOC646627 | ST6GALNAC1 |
| | DNASE1L3 | LOC652128 | SULT1A1 |
| | EDG2 | LOC96610 | TNXB |
| | ENAM | MAOA | TSPAN1 |
| | ENTPD5 | MATN2 | TTRAP |
| | ETHE1 | MEP1A | UGDH |
| | FABP1 | METTL7A | UGP2 |
| | FAM46C | MFSD4 | UGT1A1 |
| | FAM55D | MGC13057 | UGT1A3 |
| | FCGBP | MIER3 | UGT1A6 |
| | FGFR2 | MMP28 | UGT1A8 |
| | FOSB | MT1A | UGT1A9 |
| | FOXF2 | MT1F | UGT2A3 |
| | FOXP2 | MT1M | UGT2B15 |
| | FUCA1 | MUC12 | UGT2B17 |
| | GPA33 | MUC2 | VSIG2 |
| | HBA1 | MUC4 | XDH |
| | HBA2 | MUCDHL | XLKD1 |
| | HBB | MYH11 | ZCWPW2 |
| | HHLA2 | NDE1 | |

in a biological simple from said individual wherein a lower level of expression of the genes or transcripts of group (i) and/or group (ii) relative to control levels is indicative of a cancer cell or cell predisposed to the onset of a cancerous state.

In still another aspect there is provided a method of screening for the onset or predisposition to the onset of a large intestine neoplasm in an individual, said method comprising screening the level of expression of one or more genes or transcripts selected from:

the gene, genes or transcripts detected by Affymetrix probeset IDs:

| | | | |
|---|---|---|---|
| (i) | 202920_at | 222717_at | 231120_x_at |
| | 203881_s_at | 224412_s_at | 231773_at |
| | 204719_at | 225381_at | 203296_s_at |
| | 204931_at | 225575_at | 206664_at |
| | 204940_at | 227529_s_at | 211549_s_at |
| | 205433_at | 227623_at | 214598_at |
| | 206637_at | 227705_at | 219948_x_at |
| | 207080_s_at | 227827_at | 220812_s_at |
| | 207980_s_at | 228504_at | 221305_s_at |
| | 209170_s_at | 228706_s_at | 229831_at |
| | 209209_s_at | 228766_at | 231925_at |
| | 209613_s_at | 228854_at | 235146_at |
| | 220037_s_at | 228885_at | 238751_at |
| | 220376_at | 230788_at | 243278_at; and/or |
| (ii) | ADH1B | ANGPTL1 | HHLA2 |
| | SORBS2 | DMD | SORBS2 |
| | PYY | GCNT2 | CLDN23 |
| | ABCA8 | SDPR | CNTN3 |
| | RPL24 | PKIB | PLEKHC1 |
| | SI | CITED2 | LRRC19 |
| | CLDN8 | TCF21 | LIFR |
| | P2RY14 | P2RY1 | ATP1A2 |
| | PLN | ANK2 | HPGD |
| | TRPM6 | XLKD1 | GPM6B |
| | CD36 | LOC399959 | UGT1A8 |
| | BCHE | AKAP12 | FOXP2 |
| | TCEAL7 | UGT2A3 | |

in a biological sample from said individual wherein a level of expression of the genes or transcripts of group (i) and/or group (ii) which is not substantially above background levels is indicative of a neoplastic cell or a cell predisposed to the onset of a neoplastic state.

In a further aspect there is provided a method of screening for the onset or predisposition to the onset of a large intestine neoplasm in an individual, said method comprising screening the level of expression of one or more genes or transcripts selected from:
(i) the gene, genes or transcripts detected by Affymetrix probeset IDs: 209209_s_at, 225381_at, 227529 s_at, 227623_at, 227705_at; and/or
(ii) AKAP12, LOC399959, PLEKHC1, TCEAL7,
   in a biological sample from said individual wherein a lower level of expression of the genes or transcripts of group (i) and/or group (ii) which is not substantially above background levels is indicative of an adenoma cell or a cell predisposed to the onset of an adenoma state.

In yet still another further aspect there is provided a method of screening for the onset or predisposition to the onset of a large intestine neoplasm in an individual, said method comprising screening the level of expression of one or more genes or transcripts selected from:

the gene, genes or transcripts detected by Affymetrix probeset IDs:

| | | | |
|---|---|---|---|
| (i) | 203296_s_at | 219948_x_at | 231925_at |
| | 206664_at | 220812_s_at | 235146_at |
| | 211549_s_at | 221305_s_at | 238751_at |
| | 214598_at | 229831_at | 243278_at; and/or |
| (ii) | ATP1A2 | HHLA2 | SORBS2 |
| | CLDN8 | HPGD | UGT1A8 |
| | CNTN3 | P2RY1 | UGT2A3 |
| | FOXP2 | SI | |

in a biological sample from said individual wherein a lower level of expression of the genes or transcripts of group (i) and/or group (ii) which is not substantially above background levels is indicative of a cancer cell or a cell predisposed to the onset of a cancerous state.

In another further aspect there is provided a method of characterising a neoplastic cell or cellular population, which cell or cellular population is derived from the large intestine of an individual, said method comprising assessing the level of expression of one or more genes or transcripts selected from:

the gene, genes or transcripts detected by Affymetrix probeset IDs:

| | | | |
|---|---|---|---|
| (i) | 200600_at | 204006_s_at | 213428_s_at |
| | 200665_s_at | 204051_s_at | 213524_s_at |
| | 200832_s_at | 204122_at | 213869_x_at |
| | 200974_at | 204320_at | 213905_x_at |
| | 200986_at | 204475_at | 214247_s_at |
| | 201058_s_at | 204620_s_at | 215049_x_at |
| | 201069_at | 205479_s_at | 215076_s_at |
| | 201105_at | 205547_s_at | 215646_s_at |
| | 201141_at | 205828_at | 216442_x_at |
| | 201147_s_at | 207173_x_at | 217430_x_at |
| | 201150_s_at | 207191_s_at | 217762_s_at |
| | 201162_at | 208747_s_at | 217763_s_at |
| | 201163_s_at | 208782_at | 217764_s_at |
| | 201185_at | 208788_at | 218468_s_at |
| | 201261_x_at | 208850_s_at | 218469_at |
| | 201289_at | 208851_s_at | 218559_s_at |
| | 201426_sat | 209101_at | 218638_s_at |
| | 201438_at | 209156_s_at | 219087_at |
| | 201616_s_at | 209218_at | 221011_s_at |
| | 201645_at | 209395_at | 221729_at |
| | 201667_at | 209396_s_at | 221730_at |
| | 201744_s_at | 209596_at | 221731_x_at |
| | 201792_at | 209875_s_at | 37892_at |
| | 201842_s_at | 209955_s_at | 223122_s_at |
| | 201852_x_at | 210095_s_at | 223235_s_at |
| | 201859_at | 210495_x_at | 224560_at |
| | 201893_x_at | 210511_s_at | 224694_at |
| | 202237_at | 210764_s_at | 224724_at |
| | 202238_s_at | 210809_s_at | 225664_at |
| | 202283_at | 211161_s_at | 225681_at |
| | 202291_s_at | 211571_s_at | 225710_at |
| | 202310_s_at | 211719_x_at | 225799_at |
| | 202311_s_at | 211813_x_at | 226237_at |
| | 202403_s_at | 211896_s_at | 226311_at |
| | 202404_s_at | 211959_at | 226694_at |
| | 202450_s_at | 211964_at | 226777_at |
| | 202620_s_at | 211966_at | 226930_at |
| | 202766_s_at | 211980_at | 227099_s_at |
| | 202859_x_at | 211981_at | 227140_at |
| | 202878_s_at | 212077_at | 227566_at |
| | 202917_s_at | 212344_at | 229218_at |
| | 202998_s_at | 212353_at | 229802_at |
| | 203083_at | 212354_at | 231579_s_at |
| | 203325_s_at | 212464_s_at | 231766_s_at |
| | 203382_s_at | 212488_at | 231879_at |
| | 203477_at | 212489_at | 232458_at |
| | 203570_at | 212667_at | 233555_s_at |
| | 203645_s_at | 213125_at | 234994_at; and/or |
| | 203878_s_at | | |
| (ii) | COL1A2 | LGALS1 | SRGN |
| | CTHRC1 | ELOVL5 | LBH |
| | FN1 | MGP | CTGF |
| | POSTN | MMP2 | TNC |
| | SPP1 | LOXL2 | G0S2 |
| | MMP1 | MYL9 | SQLE |
| | SPARC | DCN | EFEMP1 |
| | LUM | CALD1 | APOE |
| | GREM1 | FBN1 | MSN |
| | IL8 | MMP3 | IGFBP3 |
| | IGFBP5 | IGFBP7 | SERPINF1 |
| | SFRP2 | FSTL1 | ISLR |
| | SULF1 | COL4A2 | HNT |
| | ASPN | VCAN | COL5A1 |
| | COL6A3 | SMOC2 | OLFML2B |
| | COL8A1 | HTRA1 | KIAA1913 |
| | COL12A1 | CYR61 | PALM2-AKAP2 |
| | COL5A2 | FAP | SERPING1 |
| | CDH11 | VIM | TYROBP |
| | THBS2 | TIMP2 | ACTA2 |
| | COL15A1 | SCD | COL3A1 |
| | COL11A1 | TIMP3 | PLOD2 |
| | S100A8 | AEBP1 | MMP11 |
| | FNDC1 | GJA1 | CD163 |
| | SFRP4 | NNMT | FCGR3B |
| | INHBA | COL1A1 | PLAU |
| | COL6A2 | SULF2 | MAFB |
| | ANTXR1 | COL6A1 | LOC541471 |
| | GPNMB | SPON2 | LOC387763 |
| | BGN | CTSK | CHI3L1 |
| | TAGLN | MXRA5 | THY1 |
| | COL4A1 | C1S | LOXL1 |
| | RAB31 | DKK3 | CD93 |

in said cell or cellular population wherein a lower level of expression of the genes of group (i) and/or group (ii) relative to a gastrointestinal cancer control level is indicative of an adenoma cell or a cell predisposed to the onset of an adenoma state.

In still another aspect there is provided a method of characterising a neoplastic cell or cellular population, which cell or cellular population is derived from the large intestine of an individual, said method comprising assessing the level of expression of one or more genes or transcripts selected from:

the gene, genes or transcripts detected by Affymetrix probeset IDs:

| | | | |
|---|---|---|---|
| (i) | 200884_at | 214234_s_at | 226248_s_at |
| | 203240_at | 214235_at | 226302_at |
| | 203963_at | 214433_s_at | 227676_at |
| | 204508_s_at | 215125_s_at | 227719_at |
| | 204607_at | 215867_x_at | 227725_at |
| | 204811_s_at | 217109_at | 228232_s_at |
| | 204895_x_at | 217110_s_at | 229070_at |
| | 204897_at | 218211_s_at | 231832_at |
| | 205259_at | 219543_at | 232176_at |
| | 205765_at | 219955_at | 232481_s_at |
| | 205927_s_at | 221841_s_at | 235976_at |
| | 208063_s_at | 221874_at | 236894_at |
| | 208937_s_at | 223969_s_at | 237521_x_at |
| | 210107_at | 223970_at | 242601_at; and/or |
| | 213106_at | | |
| (ii) | CLCA1 | CTSE | ATP8B1 |
| | FCGBP | C6orf105 | CACNA2D2 |
| | HMGCS2 | CKB | KLF4 |
| | RETNLB | ATP8A1 | CYP3A5P2 |
| | L1TD1 | MUC4 | CAPN9 |
| | SLITRK6 | UGT1A1 | NR3C2 |
| | VSIG2 | SELENBP1 | PBLD |
| | LOC253012 | PTGER4 | CA12 |
| | ST6GALNAC1 | MLPH | WDR51B |
| | ID1 | KIAA1324 | FAM3D |
| | CYP3A5 | | |

in a biological sample from said individual wherein a lower level of expression of the genes or transcripts of group (i) and/or group (ii) relative to a gastrointestinal adenoma control level is indicative of a cancer or a cell predisposed to the onset of a cancerous state.

In yet another aspect there is provided a method of characterising a neoplastic cell or cellular population, which cell or cellular population is derived from the large intestine of an individual, said method comprising assessing the level of expression of one or more genes or transcripts selected from:
(i) the gene or genes detected by Affymetrix probeset IDs: 202404_s_at, 212464_s_at, 210809_s_at, 225681_at; and/or
(ii) COL1 A2, FN1, POSTN, CTHRC1
   in a biological sample from said individual wherein a lower level of expression of the genes or transcripts of group (i) and/or group (ii) relative to a gastrointestinal cancer control level is indicative of an adenoma cell or a cell predisposed to the onset of an adenoma state.

In yet still another aspect, there is provided a method of characterising a neoplastic cell or cellular population, which cell or cellular population is derived from the large intestine of an individual, said method comprising assessing the level of expression of one or more genes selected from:

the gene, genes or transcripts detected by Affymetrix probeset IDs:

| | | | |
|---|---|---|---|
| (i) | 209875_s_at | 227140_at | 204475_at; and/or |
| (ii) | SPP1 | MMP1 | |

in a biological sample from said individual wherein a lower level of expression of the genes or transcripts of group (i) and/or group (ii) relative to a gastrointestinal cancer control level is indicative of an adenoma cell or a cell predisposed to the onset of an adenoma state.

In still yet another aspect the present invention is directed to a method of characterising a cell or cellular population, which cell or cellular population is derived from the large intestine of an individual, said method comprising assessing the level of expression of one or more genes or transcripts selected from:

the gene, genes or transcripts detected by Affymetrix probeset IDs:

| | | | |
|---|---|---|---|
| (i) | 200665_s_at | 226237_at | 226930_at |
| | 201744_s_at | 225664_at | 204051_s_at |
| | 218468_s_at | 221730_at | 210511_s_at |
| | 202859_x_at | 207173_x_at | 209156_s_at |
| | 211959_at | 203083_at | 224694_at |
| | 223122_s_at | 203477_at | 201141_at |
| | 212353_at | 37892_at | 213905_x_at |
| | 219087_at | 202917_s_at | 205547_s_at |
| | 201438_at; and/or | | |
| (ii) | SPARC | COL8A1 | SFRP4 |
| | LUM | COL12A1 | INHBA |
| | GREM1 | COL5A2 | COL6A2 |
| | IL8 | CDH11 | ANTXR1 |
| | IGFBP5 | THBS2 | GPNMB |
| | SFRP2 | COL15A1 | BGN |
| | SULF1 | COL11A1 | TAGLN |
| | ASPN | S100A8 | |
| | COL6A3 | FNDC1 | |

in a biological sample from said individual wherein a lower level of expression of the genes or transcripts of group (i) and/or (ii) relative to a gastrointestinal cancer control level is indicative of an adenoma or a cell predisposed to the onset of an adenoma state.

In yet another aspect the present invention is directed to a method of characterising a cell or cellular population, which cell or cellular population is derived from the large intestine of an individual, said method comprising assessing the level of expression of one or more genes or transcripts selected from:
(i) the gene, genes or transcripts detected by Affymetrix probeset IDs: 210107_at; and/or
(ii) CLCA1

in a biological sample from said individual wherein a lower level of expression of the genes or transcripts of group (i) and/or (ii) relative to a gastrointestinal adenoma control level is indicative of a cancer or a cell predisposed to the onset of a cancerous state.

In still yet another aspect the present invention is directed to a method of characterising a cell or cellular population, which cell or cellular population is derived from the large intestine of an individual, said method comprising assessing the level of expression of one or more genes or transcripts selected from:

the gene, genes or transcripts detected by Affymetrix probeset IDs:

| | | | |
|---|---|---|---|
| (i) | 203240_at | 219955_at | 242601_at |
| | 204607_at | 232481_s_at | 227725_at |
| | 223969_s_at | 228232_s_at; and/or | |
| (ii) | FCGBP | L1TD1 | LOC253012 |
| | HMGCS2 | SLITRK6 | ST6GALNAC1 |
| | RETNLB | VSIG2 | |

in a biological sample from said individual wherein a lower level of expression of the genes or transcripts of group (i) and/or (ii) relative to a gastrointestinal adenoma control level is indicative of a cancer or a cell predisposed to the onset of a cancerous state.

A further aspect of the present invention provides a method of characterising a neoplastic cell or cellular population, which cell or cellular population is derived from the large intestine of an individual, said method comprising assessing the level of expression of one or more genes or transcripts selected from:

the gene or genes detected by Affymetrix probeset IDs:

| | | | |
|---|---|---|---|
| (i) | 235976_at | 236894_at | 237521; and/or |
| (ii) | SLITRK6 | L1TD1 | |

in a biological sample from said individual wherein expression of the genes or transcripts of group (i) and/or (ii) at a level which is not substantially greater than background neoplastic tissue levels is indicative of a cancer or a cell predisposed to the onset of a cancerous state.

A related aspect of the present invention provides a molecular array, which array comprises a plurality of;
(i) nucleic acid molecules comprising a nucleotide sequence corresponding to any one or more of the neoplastic marker genes hereinbefore described or a sequence exhibiting at least 80% identity thereto or a functional derivative, fragment, variant or homologue of said nucleic acid molecule; or
(ii) nucleic acid molecules comprising a nucleotide sequence capable of hybridising to any one or more of the sequences of (i) under medium stringency conditions or a functional derivative, fragment, variant or homologue of said nucleic acid molecule; or
(iii) nucleic acid probes or oligonucleotides comprising a nucleotide sequence capable of hybridising to any one or more of the sequences of (i) under medium stringency conditions or a functional derivative, fragment, variant or homologue of said nucleic acid molecule; or
(iv) probes capable of binding to any one or more of the proteins encoded by the nucleic acid molecules of (i) or a derivative, fragment or, homologue thereof

wherein the level of expression of said marker genes of (i) or proteins of (iv) is indicative of the neoplastic state of a cell or cellular subpopulation derived from the large intestine.

### BRIEF DESCRIPTION OF THE DRAWINGS

FIG. 1 is a graphical representation of alcohol dehydrogenase IB (class I), beta polypeptide.

FIG. 2 is a graphical representation of the methylation of MAMDC2 and GPM6B in normal and neoplastic tissues and cell lines. Panel A shows the methylation level of the MAMDC2 gene as assessed by methylation specific PCR, using amplification of the CAGE gene to normalise for input DNA levels. Each point represents an individual tissue sample or cell line. Samples included DNAs from 18 colorectal cancer tissues, 12 colorectal adenomas, 22 matched normal colorectal tissues, 6 other normal tissues and a cell line and 6 colon cancer cell lines. Panel B shows the relative level of methylation of the GPM6B gene assessed by a COBRA assay. Levels of methylation were scored between 0 (no restriction enzyme digestion) and 5 (complete restriction enzyme digestion). Each point represents a single tissue sample. Samples included 14 colorectal cancer tissues, 11 colorectal adenomas and 22 matched normal tissues.

FIG. 3 is a schematic representation of predicted RNA variants derived from hCG_1815491. cDNA clones derived from map region 8579310 to 8562303 on human chromosome 16 were used to locate exon sequences. Arrows: Oligo nucleotide primer sets were designed to allow measurement of individual RNA variants by PCR. Primers covering splice junctions are shown as spanning intron sequences which is not included in the actual oligonucleotide primer sequence.

### DETAILED DESCRIPTION OF THE INVENTION

The present invention is predicated, in part, on the elucidation of gene expression profiles which characterise large intestine cellular populations in terms of their neoplastic state and, more particularly, whether they are malignant or pre-malignant. This finding has now facilitated the development of routine means of screening for the onset or predisposition to the onset of a large intestine neoplasm or characterising cellular populations derived from the large intestine based on screening for downregulation of the expression of these molecules, relative to control expression patterns and levels. To this end, in addition to assessing expression levels of the subject genes relative to normal or non-neoplastic levels, it has been determined that a proportion of these genes are not expressed in the diseased state, thereby facilitating the development of a simple qualitative test based on requiring assessment only relative to test background levels.

In accordance with the present invention, it has been determined that the genes detailed above are modulated, in terms of differential changes to their levels of expression, depending on whether the cell expressing that gene is neoplastic or not. It should be understood that reference to a gene “expression product” or “expression of a gene” is a reference to either a transcription product (such as primary RNA or mRNA) or a translation product such as protein. In this regard, one can assess changes to the level of expression of a gene either by screening for changes to the level of expression product which is produced (i.e. RNA or protein), changes to the chromatin proteins with which the gene is associated, for example the presence of histone H3 methylated on lysine at amino acid position number 9 or 27 (repressive modifications) or changes to the DNA itself which acts to downregulate expression, such as changes to the methylation of the DNA. These genes and their gene expression products, whether they be RNA transcripts, changes to the DNA which act to downregulate expression or encoded proteins, are collectively referred to as “neoplastic markers”.

Accordingly, one aspect of the present invention is directed to a method of screening for the onset or predisposition to the onset of a large intestine neoplasm in an individual, said method comprising assessing the level of expression of one or more genes or transcripts selected from:

the gene, genes or transcripts detected by Affymetrix probeset IDs:

| | | | |
|---|---|---|---|
| (i) | 200600_at | 210133_at | 227235_at |
| | 200621_at | 210139_s_at | 227265_at |
| | 200795_at | 210298_x_at | 227404_s_at |
| | 200799_at | 210299_s_at | 227529_s_at |
| | 200845_s_at | 210302_s_at | 227561_at |
| | 200859_x_at | 210495_x_at | 227623_at |
| | 200897_s_at | 210517_s_at | 227662_at |
| | 200974_at | 210764_s_at | 227705_at |
| | 200986_at | 210809_s_at | 227727_at |
| | 201041_s_at | 210946_at | 227826_s_at |
| | 201058_s_at | 210982_s_at | 227827_at |
| | 201061_s_at | 211161_s_at | 228202_at |
| | 201069_at | 211548_s_at | 228504_at |
| | 201105_at | 211596_s_at | 228507_at |
| | 201137_s_at | 211643_x_at | 228640_at |
| | 201141_at | 211644_x_at | 228706_s_at |
| | 201150_s_at | 211645_x_at | 228707_at |
| | 201289_at | 211671_s_at | 228750_at |
| | 201300_s_at | 211696_x_at | 228766_at |
| | 201324_at | 211719_x_at | 228846_at |
| | 201348_at | 211798_x_at | 228854_at |
| | 201426_s_at | 211813_x_at | 228885_at |
| | 201427_s_at | 211848_s_at | 229530_at |
| | 201438_at | 211889_x_at | 229839_at |
| | 201496_x_at | 211896_s_at | 230087_at |
| | 201497_x_at | 211959_at | 230264_s_at |
| | 201539_s_at | 211964_at | 230788_at |
| | 201540_at | 211985_s_at | 230830_at |
| | 201616_s_at | 211990_at | 231120_x_at |
| | 201617_x_at | 211991_s_at | 231579_s_at |
| | 201645_at | 212077_at | 231773_at |
| | 201667_at | 212091_s_at | 234764_x_at |
| | 201739_at | 212097_at | 234987_at |
| | 201743_at | 212136_at | 236300_at |
| | 201744_s_at | 212158_at | 236313_at |
| | 201842_s_at | 212185_x_at | 242317_at |
| | 201852_x_at | 212192_at | 200884_at |
| | 201858_s_at | 212195_at | 201495_x_at |
| | 201859_at | 212230_at | 202266_at |
| | 201865_x_at | 212233_at | 202350_s_at |
| | 201893_x_at | 212265_at | 202731_at |
| | 201920_at | 212288_at | 202741_at |
| | 201957_at | 212386_at | 202742_s_at |
| | 202007_at | 212387_at | 202768_at |
| | 202037_s_at | 212397_at | 202838_at |
| | 202069_s_at | 212414_s_at | 203058_s_at |
| | 202133_at | 212419_at | 203060_s_at |
| | 202222_s_at | 212464_s_at | 203240_at |
| | 202242_at | 212667_at | 203296_s_at |
| | 202274_at | 212671_s_at | 203343_at |
| | 202283_at | 212713_at | 203474_at |
| | 202291_s_at | 212730_at | 203638_s_at |
| | 202388_at | 212764_at | 203963_at |
| | 202555_s_at | 212859_x_at | 204018_x_at |
| | 202620_s_at | 212956_at | 204034_at |
| | 202686_s_at | 213068_at | 204036_at |
| | 202746_at | 213071_at | 204130_at |
| | 202760_s_at | 213428_s_at | 204388_s_at |
| | 202766_s_at | 213509_x_at | 204389_at |
| | 202888_s_at | 213624_at | 204508_s_at |
| | 202920_at | 213746_s_at | 204532_x_at |
| | 202953_at | 213891_s_at | 204607_at |
| | 202957_at | 214027_x_at | 204673_at |
| | 202992_at | 214038_at | 204818_at |
| | 202994_s_at | 214091_s_at | 204895_x_at |
| | 202995_s_at | 214142_at | 204897_at |
| | 203000_at | 214414_x_at | 205112_at |
| | 203001_s_at | 214505_s_at | 205259_at |
| | 203066_at | 214677_x_at | 205403_at |
| | 203131_at | 214696_at | 205480_s_at |
| | 203305_at | 214752_x_at | 205554_s_at |
| | 203382_s_at | 214768_x_at | 205593_s_at |
| | 203477_at | 214777_at | 205892_s_at |
| | 203645_s_at | 215049_x_at | 205929_at |
| | 203680_at | 215076_s_at | 206000_at |
| | 203729_at | 215118_s_at | 206094_x_at |
| | 203748_x_at | 215176_x_at | 206262_at |
| | 203766_s_at | 215193_x_at | 206377_at |
| | 203881_s_at | 215382_x_at | 206385_s_at |
| | 203908_at | 215388_s_at | 206664_at |
| | 203913_s_at | 215657_at | 207126_x_at |
| | 203914_x_at | 216207_x_at | 207245_at |
| | 203951_at | 216401_x_at | 207390_s_at |
| | 203980_at | 216442_x_at | 207392_x_at |
| | 204069_at | 216474_x_at | 207432_at |
| | 204083_s_at | 216576_x_at | 207761_s_at |
| | 204122_at | 216834_at | 208596_s_at |
| | 204135_at | 216984_x_at | 208920_at |
| | 204326_x_at | 217148_x_at | 209114_at |
| | 204438_at | 217179_x_at | 209374_s_at |
| | 204457_s_at | 217235_x_at | 209458_x_at |
| | 204570_at | 217258_x_at | 209791_at |
| | 204688_at | 217378_x_at | 210107_at |
| | 204697_s_at | 217480_x_at | 210524_x_at |
| | 204719_at | 217546_at | 210735_s_at |
| | 204745_x_at | 217757_at | 211372_s_at |
| | 204834_at | 217762_s_at | 211538_s_at |
| | 204894_s_at | 217764_s_at | 211549_s_at |
| | 204931_at | 217767_at | 211637_x_at |
| | 204938_s_at | 217897_at | 211699_x_at |
| | 204939_s_at | 217967_s_at | 211745_x_at |
| | 204940_at | 218087_s_at | 212224_at |
| | 204955_at | 218162_at | 212592_at |
| | 205097_at | 218224_at | 212741_at |
| | 205200_at | 218312_s_at | 212814_at |
| | 205267_at | 218353_at | 213317_at |
| | 205382_s_at | 218418_s_at | 213451_x_at |
| | 205412_at | 218468_s_at | 213629_x_at |
| | 205433_at | 218469_at | 213921_at |
| | 205464_at | 218559_s_at | 213953_at |
| | 205547_s_at | 218756_s_at | 214164_x_at |
| | 205683_x_at | 219014_at | 214433_s_at |
| | 205935_at | 219087_at | 214598_at |
| | 205950_s_at | 219508_at | 214916_x_at |
| | 206134_at | 219607_s_at | 215125_s_at |
| | 206143_at | 219669_at | 215299_x_at |
| | 206149_at | 219799_s_at | 215867_x_at |
| | 206198_s_at | 220026_at | 216336_x_at |
| | 206199_at | 220037_s_at | 216491_x_at |
| | 206208_at | 220376_at | 216510_x_at |
| | 206209_s_at | 220834_at | 217022_s_at |
| | 206422_at | 221541_at | 217109_at |
| | 206461_x_at | 221667_s_at | 217110_s_at |
| | 206561_s_at | 221747_at | 217165_x_at |
| | 206576_s_at | 221748_s_at | 217232_x_at |
| | 206637_at | 222043_at | 217414_x_at |
| | 206641_at | 222162_s_at | 218541_s_at |
| | 206710_s_at | 222453_at | 218546_at |
| | 206784_at | 222513_s_at | 219059_s_at |
| | 207003_at | 222717_at | 219543_at |
| | 207080_s_at | 222722_at | 219796_s_at |
| | 207134_x_at | 223121_s_at | 219948_x_at |
| | 207266_x_at | 223122_s_at | 220075_s_at |
| | 207502_at | 223235_s_at | 220266_s_at |
| | 207961_x_at | 223343_at | 220468_at |
| | 207977_s_at | 223395_at | 220645_at |
| | 207980_s_at | 223551_at | 220812_s_at |
| | 208131_s_at | 223623_at | 221004_s_at |
| | 208370_s_at | 223952_x_at | 221305_s_at |
| | 208383_s_at | 224009_x_at | 221584_s_at |
| | 208399_s_at | 224352_s_at | 221841_s_at |
| | 208450_at | 224412_s_at | 221896_s_at |
| | 208581_x_at | 224480_s_at | 223484_at |
| | 208747_s_at | 224560_at | 223597_at |
| | 208763_s_at | 224663_s_at | 223754_at |
| | 208788_at | 224694_at | 224342_x_at |
| | 208789_at | 224823_at | 224989_at |
| | 208791_at | 224836_at | 224990_at |
| | 208792_s_at | 224840_at | 225458_at |
| | 208894_at | 224959_at | 225728_at |
| | 209047_at | 224963_at | 226147_s_at |
| | 209074_s_at | 224964_s_at | 226302_at |
| | 209101_at | 225207_at | 226594_at |
| | 209116_x_at | 225242_s_at | 226654_at |
| | 209138_x_at | 225269_s_at | 226811_at |
| | 209147_s_at | 225275_at | 227052_at |
| | 209156_s_at | 225353_s_at | 227522_at |
| | 209167_at | 225381_at | 227682_at |
| | 209170_s_at | 225442_at | 227725_at |
| | 209191_at | 225575_at | 227735_s_at |
| | 209209_s_at | 225602_at | 227736_at |
| | 209210_s_at | 225604_s_at | 228133_s_at |
| | 209283_at | 225626_at | 228195_at |
| | 209301_at | 225688_s_at | 228232_s_at |
| | 209312_x_at | 225710_at | 228241_at |
| | 209335_at | 225720_at | 228469_at |
| | 209357_at | 225721_at | 228961_at |
| | 209373_at | 225782_at | 229070_at |
| | 209436_at | 225894_at | 229254_at |
| | 209457_at | 225895_at | 229659_s_at |
| | 209496_at | 226001_at | 229831_at |
| | 209498_at | 226051_at | 230595_at |
| | 209612_s_at | 226084_at | 231925_at |
| | 209613_s_at | 226103_at | 231975_s_at |
| | 209621_s_at | 226303_at | 233565_s_at |
| | 209651_at | 226304_at | 235146_at |
| | 209656_s_at | 226333_at | 235766_x_at |
| | 209667_at | 226430_at | 235849_at |
| | 209668_x_at | 226492_at | 238143_at |
| | 209687_at | 226682_at | 238750_at |
| | 209735_at | 226694_at | 238751_at |
| | 209763_at | 226818_at | 239272_at |
| | 209868_s_at | 226834_at | 241994_at |
| | 209948_at | 226841_at | 242447_at |
| | 210084_x_at | 227006_at | 242601_at |
| | 227099_s_at | 227061_at | 243278_at; and/or |
| (ii) | CLCA4 | SGK | MT1X |
| | ZG16 | CFL2 | AOC3 |
| | CA2 | C1S | PPAP2A |
| | CA1 | SELENBP1 | ZSCAN18 |
| | MS4A12 | MT1E | IVD |
| | AQP8 | ADAMTS1 | SFRP1 |
| | SLC4A4 | ITM2A | COL4A2 |
| | CEACAM7 | POU2AF1 | GPM6B |
| | TAGLN | FAM55D | EPB41L3 |
| | GUCA1B | C6orf204 | MAOA |
| | GCG | AKAP12 | DMD |
| | ADH1B | TUBB6 | MSRB3 |
| | UGT2B17 | LGALS2 | PLOD2 |
| | ADAMDEC1 | KIAA0828 | C9orf19 |
| | MT1M | MGC14376 | MDER3 |
| | AKR1B10 | PPP1R14A | XDH |
| | FN1 | MUC4 | CLDN23 |
| | MGP | PKIB | SGCE |
| | CXCL12 | PIGR | FOXF2 |
| | PDK4 | ASPN | AGR3 |
| | CA4 | A2M | IGLJ3 |
| | PYY | LOC25845 | QKI |
| | IGHA1 | LGALS1 | LOC399959 |
| | TPM2 | BCHE | ANKRD25 |
| | C6orf105 | ST6GALNAC1 | CRISPLD2 |
| | HPGD | GJA1 | ANK2 |
| | ADH1C | SCNN1B | LOC283666 |
| | CLCA1 | FABP4 | CRYAB |
| | FABP1 | F13A1 | ACAT1 |
| | ENAM | CD36 | IGL@ |
| | CFD | SPARCL1 | PBLD |
| | GUCA2B | ZCWPW2 | CCL8 |
| | FBLN1 | TNC | LIFR |
| | LOC63928 | MT1A | HLA-DRB1 |
| | ABCA8 | LOC652745 | UGP2 |
| | POSTN | MALL | IGKV1D-13 |
| | DCN | GNG2 | AP1S2 |
| | ITLN1 | DNASE1L3 | EMP3 |
| | COL6A2 | EGR1 | MMP28 |
| | FCGBP | CMBL | UGT2A3 |
| | SLC26A2 | GCNT3 | RGS5 |
| | PGM5 | SERPING1 | PTGIS |
| | DMN | MEIS1 | DUSP5 |
| | GPNMB | EDN3 | MFAP4 |
| | IGFBP5 | MSN | UGT1A6 |
| | CLEC3B | MT1G | PRKAR2B |
| | LOC253012 | TPSAB1 | HHLA2 |
| | DPT | GPX3 | LOC652128 |
| | PCK1 | CDKN2B | C3 |
| | CNN1 | FOSB | ATP2B4 |
| | HSD17B2 | HSPA1A | HBA1 |
| | PLAC8 | CYBRD1 | TCF21 |
| | TMEM47 | PTGER4 | PPID |
| | OGN | MAG1 | PPAP2B |
| | CALD1 | BEST2 | SPON1 |
| | ACTG2 | HLA-DQA1 | PHLDB2 |
| | MGC4172 | PRIMA1 | RARRES2 |
| | MAB21L2 | MT1F | ETHE1 |
| | RPL24 | MAFB | MMP2 |
| | ABCG2 | FAM107A | SRI |
| | CCDC80 | PRKACB | CNTN3 |
| | UGT1A1 | SELM | RGS2 |
| | MRC1 | TYROBP | COL6A1 |
| | HSD11B2 | TNS1 | FBN1 |
| | ANPEP | MYH11 | MXD1 |
| | MATN2 | ITM2C | PLCE1 |
| | PRNP | CES2 | KCNMB1 |
| | ABI3BP | MS4A4A | CALM1 |
| | HLA-C | PDGFRA | HLA-DPB1 |
| | NDE1 | CA12 | SMOC2 |
| | SRPX | FKBP5 | LOC285382 |
| | WWTR1 | HSPB8 | CLIC5 |
| | HMGCS2 | TPSB2 | APOE |
| | LOC646627 | FGL2 | SERPINF1 |
| | KRT20 | C1QB | PPP1R12B |
| | KLF4 | ANGPTL1 | HSPB6 |
| | FHL1 | MEP1A | FNBP1 |
| | ARL14 | GUCY1A3 | C4orf34 |
| | LUM | UGDH | SORBS2 |
| | SORBS1 | DUSP1 | GPA33 |
| | METTL7A | C2orf40 | GALNAC4S-6ST |
| | FAM129A | PLN | CFHR1 |
| | SCARA5 | UGT2B15 | MGC13057 |
| | SI | PDLIM3 | C10orf56 |
| | ACTA2 | TP53INP2 | SULT1A1 |
| | CD177 | ATP8B1 | TTRAP |
| | C10orf99 | ANK3 | CCL28 |
| | COL15A1 | CTGF | IDH3A |
| | NR3C2 | MUCDHL | EDG2 |
| | DHRS9 | SDPR | UGT1A8 |
| | LMOD1 | COL14A1 | RAB27A |
| | EFEMP1 | DSCR1 | ANTXR1 |
| | GREM1 | CITED2 | EMP1 |
| | IL1R2 | MT1H | CSRP1 |
| | LOC387763 | NEXN | PLEKHC1 |
| | TIMP3 | MUC2 | LOC572558 |
| | MYLK | NID1 | FOXP2 |
| | CLDN8 | HBB | HSPA2 |
| | RDX | GCNT2 | ATP1A2 |
| | TSPAN7 | C20orf118 | TNXB |
| | TNFRSF17 | SLC20A1 | FUCA1 |
| | SYNPO2 | CD14 | MRGPRF |
| | VIM | KCTD12 | HIGD1A |
| | SMPDL3A | RBMS1 | MFSD4 |
| | P2RY14 | PTRF | AXL |
| | CHGA | TSPAN1 | AQP1 |
| | C15orf48 | UGT1A9 | MAP1B |
| | COL3A1 | COX7A1 | PALLD |
| | CYR61 | MUC12 | MPEG1 |
| | TRPM6 | PDCD4 | KLHL5 |
| | OSTbeta | CAV1 | TCEAL7 |
| | IGLV1-44 | FAM46C | FILIP1L |
| | VSIG2 | LRIG1 | IQGAP2 |
| | IGHM | HLA-DPA1 | PRDX6 |
| | LRRC19 | C1orf115 | RAB31 |
| | CD163 | HBA2 | LOC96610 |
| | CEACAM1 | EDIL3 | FGFR2 |
| | TIMP2 | DES | PAPSS2 |
| | ENTPD5 | MT2A | XLKD1 |
| | DDR2 | KCNMA1 | SMTN |
| | CHRDL1 | GAS1 | C8orf4 |
| | SRGN | TBC1D9 | SDCBP2 |
| | PDE9A | C7 | CCL11 |
| | PMP22 | P2RY1 | ELOVL5 |
| | FLNA | NR3C1 | FOXF1 |
| | STMN2 | STOM | RELL1 |
| | MYL9 | CKB | PNMA1 |
| | SEMA6D | CLU | LOC339562 |
| | PADI2 | SLC26A3 | PALM2-AKAP2 |
| | SEPPI | SDC2 | PAG1 |
| | TGFB1I1 | SST | HCLS1 |
| | SFRP2 | HLA-DRA | RGS1 |
| | UGT1A3 | TSC22D3 | FXYD6 |
| | MS4A7 | IL6ST | OLFML3 |
| | ALDH1A1 | C1QC | COL6A3 |

in a biological sample from said individual wherein a lower level of expression of the genes or transcripts of group (i) and/or group (ii) relative to control levels is indicative of a neoplastic large intestine cell or a cell predisposed to the onset of a neoplastic state.

In one embodiment, said expression is assessed by screening for DNA changes which impact on methylation, in particular hypermethylation. In another embodiment expression is assessed by the association of DNA with chromatin proteins carrying repressive modifications, for example, methylation of lysines 9 or 27 of the histone H3.

Reference to “large intestine” should be understood as a reference to a cell derived from one of the six anatomical regions of the large intestine, which regions commence after the terminal region of the ileum, these being:
(i) the cecum;
(ii) the ascending colon;
(iii) the transverse colon;
(iv) the descending colon;
(v) the sigmoid colon; and
(vi) the rectum.

Reference to “neoplasm” should be understood as a reference to a lesion, tumour or other encapsulated or unencapsulated mass or other form of growth which comprises neoplastic cells. A “neoplastic cell” should be understood as a reference to a cell exhibiting abnormal growth. The term “growth” should be understood in its broadest sense and includes reference to proliferation. In this regard, an example of abnormal cell growth is the uncontrolled proliferation of a cell. Another example is failed apoptosis in a cell, thus prolonging its usual life span. The neoplastic cell may be a benign cell or a malignant cell. In a preferred embodiment, the subject neoplasm is an adenoma or an adenocarcinoma. Without limiting the present invention to any one theory or mode of action, an adenoma is generally a benign tumour of epithelial origin which is either derived from epithelial tissue or exhibits clearly defined epithelial structures. These structures may take on a glandular appearance. It can comprise a malignant cell population within the adenoma, such as occurs with the progression of a benign adenoma to a malignant adenocarcinoma.

Preferably, said neoplastic cell is an adenoma or adenocarcinoma and even more preferably a colorectal adenoma or adenocarcinoma.

Each of the genes and transcripts detailed in sub-paragraphs (i) and (ii), above, would be well known to the person of skill in the art, as would their encoded proteins. The identification of the expression products of these genes and transcripts as markers of neoplasia occurred by virtue of differential expression analysis using Affymetrix HGU133A or HGU133B gene chips. To this end, each gene chip is characterised by approximately 45,000 probe sets which detect the RNA transcribed from the genome. On average, approximately 11 probe pairs detect overlapping or consecutive regions of the RNA transcript. In general, the genes from which the RNA transcripts described herein are identifiable by the Affymetrix probesets are well known and characterised genes. However, to the extent that some of the probesets detect RNA transcripts which are not yet defined, these transcripts are indicated as “the gene, genes or transcripts detected by Affymetrix probe x”. In some cases a number of genes may be detectable by a single probeset. It should be understood, however, that this is not intended as a limitation as to how the expression level of the subject gene or transcript can be detected. In the first instance, it would be understood that the subject gene transcript is also detectable by other probesets which would be present on the Affymetrix gene chip. The reference to a single probeset is merely included as an identifier of the gene transcript of interest. In terms of actually screening for the transcript, however, one may utilise a probe or probeset directed to any region of the transcript and not just to the 3-terminal 600 bp transcript region to which the Affymetrix probes are often directed.

Reference to each of the genes and transcripts detailed above and their transcribed and translated expression products should therefore be understood as a reference to all forms of these molecules and to fragments or variants thereof. As would be appreciated by the person of skill in the art, some genes are known to exhibit allelic variation between individuals. Accordingly, the present invention should be understood to extend to such variants which, in terms of the present diagnostic applications, achieve the same outcome despite the fact that minor genetic variants between the actual nucleic acid sequences may exist between individuals or that within one individual there may exist 2 or more splice variants of the subject gene. The present invention should therefore be understood to extend to all forms of RNA (eg mRNA, primary RNA transcript, miRNA, etc), cDNA and peptide isoforms which arise from alternative splicing or any other mutation, polymorphic or allelic variation. It should also be understood to include reference to any subunit polypeptides such as precursor forms which may be generated, whether existing as a monomer, multimer, fusion protein or other complex.

To this end, in terms of the genes encompassed by the present invention, means for determining the existence of such variants, and characterising same, are described in Example 6. To the extent that the genes of the present invention are described by reference to an Affymetrix probeset, Table 6 provides details of the nucleic acid sequence to which each probe set is directed. Based on this information, the skilled person could, as a matter of routine procedure, identify the gene in respect of which that sequence forms part. A typical protocol for doing this is also outlined in Example 6.

It should be understood that the “individual” who is the subject of testing may be any human or non-human mammal. Examples of non-human mammals includes primates, livestock animals (e.g. horses, cattle, sheep, pigs, donkeys), laboratory test animals (e.g. mice, rats, rabbits, guinea pigs), companion animals (e.g. dogs, cats) and captive wild animals (e.g. deer, foxes). Preferably the mammal is a human.

The method of the present invention is predicated on the comparison of the level of the neoplastic markers of a biological sample with the control levels of these markers. The “control level” may be either a “normal level”, which is the level of marker expressed by a corresponding large intestine cell or cellular population which is not neoplastic.

The normal (or “non-neoplastic”) level may be determined using tissues derived from the same individual who is the subject of testing. However, it would be appreciated that this may be quite invasive for the individual concerned and it is therefore likely to be more convenient to analyse the test results relative to a standard result which reflects individual or collective results obtained from individuals other than the patient in issue. This latter form of analysis is in fact the preferred method of analysis since it enables the design of kits which require the collection and analysis of a single biological sample, being a test sample of interest. The standard results which provide the normal level may be calculated by any suitable means which would be well known to the person of skill in the art. For example, a population of normal tissues can be assessed in terms of the level of the neoplastic markers of the present invention, thereby providing a standard value or range of values against which all future test samples are analysed. It should also be understood that the normal level may be determined from the subjects of a specific cohort and for use with respect to test samples derived from that cohort. Accordingly, there may be determined a number of standard values or ranges which correspond to cohorts which differ in respect of characteristics such as age, gender, ethnicity or health status. Said “normal level” may be a discrete level or a range of levels. A decrease in the expression level of the subject genes relative to normal levels is indicative of the tissue being neoplastic.

Without limiting the present invention to any one theory or mode of action, although each of the genes or transcripts hereinbefore described is differentially expressed, either singly or in combination, as between neoplastic versus non-neoplastic cells of the large intestine, and is therefore diagnostic of the existence of a large intestine neoplasm, the expression of some of these genes was found to exhibit particularly significant levels of sensitivity, specificity and positive and negative predictive value. Accordingly, in a preferred embodiment one would screen for and assess the expression level of one or more of these genes. To this end, and without limiting the present invention to any one theory or mode of action, the following markers were determined to be expressed in neoplastic tissue at a level of 3-11 fold less than non-neoplastic tissue, when assessed by virtue of the method exemplified herein:

| Fold | Gene, genes or transcripts | |
|---|---|---|
| Decrease | detected by Affymetrix Probe No: | Gene |
| 11 | 220026_at | CLCA4 |
| 10 | 214142_at | ZG16 |
| 9 | 209301_at | CA1 |
| | 205950_s_at | CA2 |
| 8 | 220834_at | MS4A12 |
| 7 | 206784_at | AQP8 |
| 6 | 203908_at | SLC4A4 |
| | 206198_s_at | CEACAM7 |
| | 205547_s_at | TAGLN |
| | 207003_at | GUCA1B |
| | 206422_at | GCG |
| | 209613_s_at | ADH1B |
| | 207245_at | UGT2B17 |
| 5 | 206134_at | ADAMDEC1 |
| | 217546_at | MT1M |
| | 206561_s_at | AKR1B10 |
| | 211719_x_at | FN1 |
| | 202291_s_at | MGP |
| | 209687_at | CXCL12 |
| 4 | 225207_at | PDK4 |
| | 206208_at | CA4 |
| | 207080_s_at | PYY |
| | 215118_s_at | IGHA1 |
| | 204083_s_at | TPM2 |
| | 229070_at | C6orf105 |
| | 211548_s_at | HPGD |
| | 206262_at | ADH1C |
| | 210107_at | CLCA1 |
| | 205892_s_at | FABP1 |
| | 212592_at | ENAM |
| | 205382_s_at | CFD |
| | 207502_at | GUCA2B |
| | 202995_s_at | FBLN1 |
| | 206149_at | LOC63928 |
| | 204719_at | ABCA8 |
| 3 | 210809_s_at | POSTN |
| | 201893_x_at | DCN |
| | 223597_at | ITLN1 |
| | 209156_s_at | COL6A2 |
| | 203240_at | FCGBP |
| | 224963_at | SLC26A2 |
| | 226303_at | PGM5 |
| | 212730_at | DMN |
| | 201141_at | GPNMB |
| | 211959_at | IGFBP5 |
| | 205200_at | CLEC3B |
| | 242601_at | LOC253012 |
| | 213068_at | DPT |
| | 208383_s_at | PCK1 |
| | 203951_at | CNN1 |
| | 204818_at | HSD17B2 |
| | 219014_at | PLAC8 |
| | 209656_s_at | TMEM47 |
| | 222722_at | OGN |
| | 201617_x_at | CALD1 |
| | 202274_at | ACTG2 |
| | 218756_s_at | MGC4172 |
| | 210302_s_at | MAB21L2 |
| | 228885_at | RPL24 |
| | 209735_at | ABCG2 |
| | 228504_at | CCDC80 |
| | 225242_s_at | UGT1A1 |
| | 215125_s_at | MRC1 |
| | 204438_at | HSD11B2 |
| | 204130_at | ANPEP |
| | 202888_s_at | MATN2 |
| | 202350_s_at | PRNP |
| | 201300_s_at | ABI3BP |
| | 223395_at | HLA-C |
| | 214768_x_at | NDE1 |
| | 228133_s_at | SRPX |
| | 204955_at | WWTR1 |
| | 202133_at | HMGCS2 |
| | 204607_at | LOC646627 |
| | 238143_at | KRT20 |
| | 213953_at | KLF4 |
| | 220266_s_at | FHL1 |
| | 210299_s_at | ARL14 |
| | 220468_at | LUM |
| | 201744_s_at | SORBS1 |
| | 218087_s_at | METTL7A |
| | 207761_s_at | FAM129A |
| | 217967_s_at | SCARA5 |
| | 229839_at | SI |
| | 206664_at | ACTA2 |
| | 200974_at | CD177 |
| | 219669_at | C10orf99 |
| | 227736_at | COL15A1 |
| | 203477_at | NR3C2 |
| | 205259_at | |

There is therefore more particularly provided a method of screening for the onset or predisposition to the onset of a large intestine neoplasm in an individual, said method comprising assessing the level of expression of one or more genes or transcripts selected from:
(i) the gene, genes or transcripts detected by Affymetrix probeset IDs: 220026 at; and/or
(ii) CLCA4

in a biological sample from said individual wherein a lower level of expression of the gene or transcripts of group (i) and/or group (ii) relative to control levels is indicative of a neoplastic large intestine cell or a cell predisposed to the onset of a neoplastic state.

Preferably, said control level is a non-neoplastic level.

In another embodiment, there is provided a method of screening for the onset or predisposition to the onset of a large intestine neoplasm in an individual, said method comprising assessing the level of expression of one or more genes or transcripts selected from:
(i) the gene, genes or transcripts detected by Affymetrix probeset IDs: 214142_at; and/or
(ii) ZG16
   in a biological sample from said individual wherein a lower level of expression of the genes or transcripts of group (i) and/or group (ii) relative to control levels is indicative of a neoplastic large intestine cell or a cell predisposed to the onset of a neoplastic state.

Preferably, said control level is a non-neoplastic level.

In yet another embodiment there is provided a method of screening for the onset or predisposition to the onset of a large intestine neoplasm in an individual, said method comprising assessing the level of expression of one or more genes or transcripts selected from:
(i) the gene, genes or transcripts detected by Affymetrix probeset IDs: 209301_at 205950 s_at; and/or
(ii) CA2 CA1
   in a biological sample from said individual wherein a lower level of expression of the genes or transcripts of group (i) and/or group (ii) relative to control levels is indicative of a neoplastic large intestine cell or a cell predisposed to the onset of a neoplastic state.

Preferably, said control level is a non-neoplastic level.

In still yet another preferred embodiment, there is provided a method of screening for the onset or predisposition to the onset of a large intestine neoplasm in an individual, said method comprising assessing the level of expression of one or more genes or transcripts selected from:
(i) the gene, genes or transcripts detected by Affymetrix probeset IDs: 220834_at; and/or
(ii) MS4A12
   in a biological sample from said individual wherein a lower level of expression of the genes or transcripts of group (i) and/or group (ii) relative to control levels is indicative of a neoplastic large intestine cell or a cell predisposed to the onset of a neoplastic state.

Preferably, said control level is a non-neoplastic level.

In yet still another preferred embodiment, there is provided a method of screening for the onset or predisposition to the onset of a large intestine neoplasm in an individual, said method comprising assessing the level of expression of one or more genes or transcripts selected from:
(i) the gene, genes or transcripts detected by Affymetrix probeset IDs: 206784_at; and/or
(ii) AQP8
   in a biological sample from said individual wherein a lower level of expression of the genes or transcripts of group (i) and/or group (ii) relative to control levels is indicative of a neoplastic large intestine cell or a cell predisposed to the onset of a neoplastic state.

Preferably, said control level is a non-neoplastic level.

In a further embodiment, there is provided a method of screening for the onset or predisposition to the onset of a large intestine neoplasm in an individual, said method comprising assessing the level of expression of one or more genes or transcripts selected from:
(i) (1) the gene, genes or transcripts detected by Affymetrix probeset IDs: 203908_at, 206198_s_at, 205547_s_at, 207003_at, 206422_at, 209613_s_at, 207245_at; and/or
(ii) SLC4A4, CEACAM7, TAGLN, GUCA1B, GCG, ADH1B, UGT2B17
   in a biological sample from said individual wherein a lower level of expression of the genes or transcripts of group (i) and/or group (ii) relative to control levels is indicative of a neoplastic large intestine cell or a cell predisposed to the onset of a neoplastic state.

Preferably, said control level is a non-neoplastic level.

In another further embodiment, there is provided a method of screening for the onset or predisposition to the onset of a large intestine neoplasm in an individual, said method comprising assessing the level of expression of one or more genes or transcripts selected from:
(i) the gene, genes or transcripts detected by Affymetrix probeset IDs: 206134_at, 217546_at, 206561_s_at, 211719_x_at, 202291_s_at, 209687_at; and/or
(ii) ADAMDEC1, MT1M, AKR1B10, FN1, MGP, CXCL12
   in a biological sample from said individual wherein a lower level of expression of the genes or transcripts of group (i) and/or group (ii) relative to control levels is indicative of a neoplastic large intestine cell or a cell predisposed to the onset of a neoplastic state.

Preferably, said control level is a non-neoplastic level.

In still another further embodiment, there is provided a method of screening for the onset or predisposition to the onset of a large intestine neoplasm in an individual, said method comprising screening the level of expression of one or more genes or transcripts selected from:

the gene, genes or transcripts detected by Affymetrix probeset IDs:

| | | | |
|---|---|---|---|
| (i) | 225207_at | 211548_s_at | 205382_s_at |
| | 206208_at | 206262_at | 207502_at |
| | 207080_s_at | 210107_at | 202995_s_at |
| | 215118_s_at | 205892_s_at | 206149_at |
| | 204083_s_at | 212592_at | 204719_at |
| | 229070_at; and/or | | |
| (ii) | PDK4 | HPGD | CFD |
| | CA4 | ADH1C | GUCA2B |
| | PYY | CLCA1 | FBLN1 |
| | IGHA1 | FABP1 | LOC63928 |
| | TPM2 | ENAM | ABCA8 |
| | C6orf105 | | |

in a biological sample from said individual wherein a lower level of expression of the genes or transcripts of group (i) and/or group (ii) relative to control levels is indicative of a neoplastic large intestine cell or a cell predisposed to the onset of a neoplastic state.

Preferably, said control level is a non-neoplastic level.

In yet still yet another further embodiment, there is provided a method of screening for the onset or predisposition to the onset of a large intestine neoplasm in an individual, said method comprising screening the level of expression of one or more genes or transcripts selected from:

the gene, genes or transcripts detected by Affymetrix probeset IDs:

| | | | |
|---|---|---|---|
| (i) | 210809_s_at | 201617_x_at | 202133_at |
| | 201893_x_at | 202274_at | 204607_at |
| | 223597_at | 218756_s_at | 238143_at |
| | 209156_s_at | 210302_s_at | 213953_at |
| | 203240_at | 228885_at | 220266_s_at |
| | 224963_at | 209735_at | 210299_s_at |
| | 226303_at | 228504_at | 220468_at |
| | 212730_at | 225242_s_at | 201744_s_at |
| | 201141_at | 215125_s_at | 218087_s_at |
| | 211959_at | 204438_at | 207761_s_at |
| | 205200_at | 204130_at | 217967_s_at |
| | 242601_at | 202888_s_at | 229839_at |
| | 213068_at | 202350_s_at | 206664_at |
| | 208383_s_at | 201300_s_at | 200974_at |
| | 203951_at | 223395_at | 219669_at |
| | 204818_at | 214768_x_at | 227736_at |
| | 219014_at | 228133_s_at | 203477_at |
| | 209656_s_at | 204955_at | 205259_at |
| | 222722_at; and/or | | |
| (ii) | POSTN | OGN | WWTR1 |
| | DCN | CALD1 | HMGCS2 |
| | ITLN1 | ACTG2 | LOC646627 |
| | COL6A2 | MGC4172 | KRT20 |
| | FCGBP | MAB21L2 | KLF4 |
| | SLC26A2 | RPL24 | FHL1 |
| | PGM5 | ABCG2 | ARL14 |
| | DMN | CCDC80 | LUM |
| | GPNMB | UGT1A1 | SORBS1 |
| | IGFBP5 | MRC1 | METTL7A |
| | CLEC3B | HSD11B2 | FAM129A |
| | LOC253012 | ANPEP | SCARA5 |
| | DPT | MATN2 | SI |
| | PCK1 | PRNP | ACTA2 |
| | CNN1 | ABI3BP | CD177 |
| | HSD17B2 | HLA-C | C10orf99 |
| | PLAC8 | NDE1 | COL15A1 |
| | TMEM47 | SRPX | NR3C2 |

in a biological sample from said individual wherein a lower level of expression of the genes or transcripts of group (i) and/or group (ii) relative to control levels is indicative of a neoplastic large intestine cell or a cell predisposed to the onset of a neoplastic state.

Preferably, said control level is a non-neoplastic level.

According to these aspects of the present invention, said large intestine tissue is preferably colorectal tissue.

In one embodiment, said expression is assessed by screening for DNA changes which impact on methylation, in particular hypermethylation. In another embodiment, expression is assessed by the association of DNA with chromatin proteins carrying repressive modifications, for example, methylation of lysines 9 or 27 of histone H3.

The detection method of the present invention can be performed on any suitable biological sample. To this end, reference to a “biological sample” should be understood as a reference to any sample of biological material derived from an animal such as, but not limited to, cellular material, biofluids (eg. blood), faeces, tissue specimens (such as biopsy specimens), surgical specimens or fluid which has been introduced into the body of an animal and subsequently removed (such as, for example, the solution retrieved from an enema wash). The biological sample which is tested according to the method of the present invention may be tested directly or may require some form of treatment prior to testing. For example, a biopsy or surgical sample may require homogenisation prior to testing or it may require sectioning for in situ testing of the qualitative expression levels of individual genes. Alternatively, a cell sample may require permeabilisation prior to testing. Further, to the extent that the biological sample is not in liquid form, (if such form is required for testing) it may require the addition of a reagent, such as a buffer, to mobilise the sample.

To the extent that the neoplastic marker gene expression product is present in a biological sample, the biological sample may be directly tested or else all or some of the nucleic acid material present in the biological sample may be isolated prior to testing. In yet another example, the sample may be partially purified or otherwise enriched prior to analysis. For example, to the extent that a biological sample comprises a very diverse cell population, it may be desirable to enrich for a sub-population of particular interest. It is within the scope of the present invention for the target cell population or molecules derived therefrom to be pretreated prior to testing, for example, inactivation of live virus or being run on a gel. It should also be understood that the biological sample may be freshly harvested or it may have been stored (for example by freezing) prior to testing or otherwise treated prior to testing (such as by undergoing culturing).

The choice of what type of sample is most suitable for testing in accordance with the method disclosed herein will be dependent on the nature of the situation. Preferably, said sample is a faecal (stool) sample, enema wash, surgical resection, tissue or blood specimen.

In a related aspect, it has been determined that certain of the markers hereinbefore defined are more indicative of adenoma development versus cancer development or vice versa. This is an extremely valuable finding since it enables one to more specifically characterise the likely nature of a neoplasm which is detected by virtue of the method of the present invention.

Accordingly, in a related aspect the present invention is directed to a method of screening for the onset or predisposition to the onset of a large intestine neoplasm in an individual, said method comprising screening the level of expression of one or more genes or transcripts selected from:

the gene, genes or transcripts detected by Affymetrix probeset IDs:

| | | | |
|---|---|---|---|
| (i) | 200600_at | 208788_at | 215382_x_at |
| | 200665_s_at | 208789_at | 215388_s_at |
| | 200799_at | 208894_at | 216442_x_at |
| | 200845_s_at | 209047_at | 216474_x_at |
| | 200859_x_at | 209101_at | 216834_at |
| | 200897_s_at | 209138_x_at | 217480_x_at |
| | 200974_at | 209147_s_at | 217757_at |
| | 200986_at | 209156_s_at | 217762_s_at |
| | 201041_s_at | 209191_at | 217764_s_at |
| | 201061_s_at | 209209_s_at | 217767_at |
| | 201069_at | 209210_s_at | 217897_at |
| | 201105_at | 209312_x_at | 218162_at |
| | 201137_s_at | 209335_at | 218224_at |
| | 201141_at | 209436_at | 218312_s_at |
| | 201150_s_at | 209457_at | 218353_at |
| | 201289_at | 209496_at | 218418_s_at |
| | 201300_s_at | 209621_s_at | 218468_s_at |
| | 201426_s_at | 209651_at | 218469_at |
| | 201438_at | 209656_s_at | 218559_s_at |
| | 201616_s_at | 209868_s_at | 219087_at |
| | 201617_x_at | 210084_x_at | 219607_s_at |
| | 201645_at | 210133_at | 221541_at |
| | 201667_at | 210139_s_at | 222043_at |
| | 201743_at | 210495_x_at | 222453_at |
| | 201744_s_at | 210517_s_at | 222513_s_at |
| | 201842_s_at | 210764_s_at | 223121_s_at |
| | 201852_x_at | 210809_s_at | 223122_s_at |
| | 201858_s_at | 210982_s_at | 223235_s_at |
| | 201859_at | 211161_s_at | 223343_at |
| | 201865_x_at | 211596_s_at | 224560_at |
| | 201893_x_at | 211671_s_at | 224694_at |
| | 201920_at | 211719_x_at | 224840_at |
| | 202007_at | 211813_x_at | 224964_s_at |
| | 202069_s_at | 211896_s_at | 225242_s_at |
| | 202133_at | 211959_at | 225269_s_at |
| | 202283_at | 211964_at | 225353_s_at |
| | 202291_s_at | 211985_s_at | 225381_at |
| | 202403_s_at | 211990_at | 225442_at |
| | 202620_s_at | 211991_s_at | 225602_at |
| | 202686_s_at | 212077_at | 225604_s_at |
| | 202760_s_at | 212091_s_at | 225626_at |
| | 202766_s_t | 212136_at | 225688_s_at |
| | 202953_at | 212158_at | 225710_at |
| | 202957_at | 212185_x_at | 226001_at |
| | 202994_s_at | 212195_at | 226051_at |
| | 202995_s_at | 212230_at | 226084_at |
| | 203066_at | 212233_at | 226103_at |
| | 203131_at | 212265_at | 226430_at |
| | 203305_at | 212386_at | 226682_at |
| | 203382_s_at | 212387_at | 226694_at |
| | 203477_at | 212397_at | 226818_at |
| | 203645_s_at | 212414_s_at | 226834_at |
| | 203680_at | 212419_at | 226841_at |
| | 203729_at | 212464_s_at | 227061_at |
| | 203748_x_at | 212667_at | 227099_s_at |
| | 204069_at | 212671_s_at | 227235_at |
| | 204122_at | 212713_at | 227404_s_at |
| | 204135_at | 212764_at | 227529_s_at |
| | 204438_at | 212956_at | 227561_at |
| | 204457_s_at | 213428_s_at | 227623_at |
| | 204570_at | 213509_x_at | 227705_at |
| | 204688_at | 213746_s_at | 227727_at |
| | 205412_at | 213891_s_at | 228507_at |
| | 205683_x_at | 214038_at | 228750_at |
| | 205935_at | 214677_x_at | 228846_at |
| | 207134_x_at | 214752_x_at | 229530_at |
| | 207266_x_at | 215049_x_at | 230264_s_at |
| | 208131_s_at | 215076_s_at | 231579_s_at |
| | 208370_s_at | 215193_x_at | 234987_at; and/or |
| | 208747_s_at | | |
| (ii) | A2M | FBN1 | PALLD |
| | ACAT1 | FILIP1L | PALM2-AKAP2 |
| | ACTA2 | FKBP5 | PDGFRA |
| | AKAP12 | FLNA | PDLIM3 |
| | ANKRD25 | FN1 | PHLDB2 |
| | ANTXR1 | FOXF1 | PLEKHC1 |
| | AP1S2 | FXYD6 | PLOD2 |
| | APOE | GALNAC4S-6ST | PMP22 |
| | AQP1 | GAS1 | PNMA1 |
| | ASPN | GJA1 | POSTN |
| | ATP2B4 | GNG2 | PPAP2A |
| | AXL | GPNMB | PPAP2B |
| | C10orf56 | GREM1 | PRDX6 |
| | C1QB | GUCY1A3 | PRKAR2B |
| | C1QC | HCLS1 | PRNP |
| | C1S | HLA-DPA1 | PTGIS |
| | C20orf118 | HLA-DPB1 | PTRF |
| | C3 | HLA-DQA1 | QKI |
| | C9orf19 | HLA-DRA | RAB31 |
| | CALD1 | HLA-DRB1 | RARRES2 |
| | CALM1 | HSPA1A | RBMS1 |
| | CCDC80 | IDH3A | RDX |
| | CCL11 | IGFBP5 | RELL1 |
| | CCL8 | IGL@ | RGS1 |
| | CD14 | IGLJ3 | RGS5 |
| | CD163 | IL6ST | SDC2 |
| | CES2 | KLHL5 | SELM |
| | CFHR1 | LGALS1 | SEPT6 |
| | CLU | LOC283666 | SERPINF1 |
| | COL14A1 | LOC339562 | SERPING1 |
| | COL15A1 | LOC387763 | SFRP2 |
| | COL1A2 | LOC399959 | SGCE |
| | COL3A1 | LRIG1 | SLC20A1 |
| | COL4A2 | LUM | SMOC2 |
| | COL6A1 | MAFB | SORBS1 |
| | COL6A2 | MAP1B | SPARC |
| | COL6A3 | MEIS1 | SPON1 |
| | COX7A1 | MFAP4 | SRGN |
| | CRISPLD2 | MGP | STOM |
| | CTGF | MMP2 | TBC1D9 |
| | CYBRD1 | MPEG1 | TCEAL7 |
| | CYR61 | MRC1 | TGFB1I1 |
| | DCN | MRGPRF | TIMP2 |
| | DDR2 | MS4A4A | TIMP3 |
| | DSCR1 | MS4A7 | TMEM47 |
| | DUSP1 | MSN | TNC |
| | DUSP5 | MT2A | TPSAB1 |
| | EFEMP1 | MXD1 | TPSB2 |
| | EGR1 | NEXN | TUBB6 |
| | ELOVL5 | NID1 | TYROBP |
| | EMP3 | NR3C1 | VIM |
| | F13A1 | OLFML3 | WWTR1 |
| | FBLN1 | PAG1 | ZSCAN18 |

in a biological sample from said individual wherein a lower level of expression of the genes or transcripts of group (i) and/or group (ii) relative to control levels is indicative of an adenoma cell or a cell predisposed to the onset of an adenoma state.

In another preferred embodiment of this aspect of the present invention there is provided a method of screening for the onset or predisposition to the onset of a large intestine neoplasm in an individual, said method comprising screening the level of expression of one or more genes or transcripts selected from:

the gene or genes detected by Affymetrix probeset IDs:

| | | | |
|---|---|---|---|
| (i) | 200884_at | 208596_s_at | 220812_s_at |
| | 201495_x_at | 208920_at | 221004_s_at |
| | 202266_at | 209114_at | 221305_s_at |
| | 202350_s_at | 209374_s_at | 221584_s_at |
| | 202731_at | 209458_x_at | 221841_s_at |
| | 202741_at | 209791_at | 221896_s_at |
| | 202742_s_at | 210107_at | 223484_at |
| | 202768_at | 210524_x_at | 223597_at |
| | 202838_at | 210735_s_at | 223754_at |
| | 203058_s_at | 211372_s_at | 224342_x_at |
| | 203060_s_at | 211538_s_at | 224989_at |
| | 203240_at | 211549_s_at | 224990_at |
| | 203296_s_at | 211637_x_at | 225458_at |
| | 203343_at | 211699_x_at | 225728_at |
| | 203474_at | 211745_x_at | 226147_s_at |
| | 203638_s_at | 212224_at | 226302_at |
| | 203963_at | 212592_at | 226594_at |
| | 204018_x_at | 212741_at | 226654_at |
| | 204034_at | 212814_at | 226811_at |
| | 204036_at | 213317_at | 227052_at |
| | 204130_at | 213451_x_at | 227522_at |
| | 204388_s_at | 213629_x_at | 227682_at |
| | 204389_at | 213921_at | 227725_at |
| | 204508_s_at | 213953_at | 227735_s_at |
| | 204532_x_at | 214164_x_at | 227736_at |
| | 204607_at | 214433_s_at | 228133_s_at |
| | 204673_at | 214598_at | 228195_at |
| | 204818_at | 214916_x_at | 228232_s_at |
| | 204895_x_at | 215125_s_at | 228241_at |
| | 204897_at | 215299_x_at | 228469_at |
| | 205112_at | 215867_x_at | 228961_at |
| | 205259_at | 216336_x_at | 229070_at |
| | 205403_at | 216491_x_at | 229254_at |
| | 205480_s_at | 216510_x_at | 229659_s_at |
| | 205554_s_at | 217022_s_at | 229831_at |
| | 205593_s_at | 217109_at | 230595_at |
| | 205892_s_at | 217110_s_at | 231925_at |
| | 205929_at | 217165_x_at | 231975_s_at |
| | 206000_at | 217232_x_at | 233565_s_at |
| | 206094_x_at | 217414_x_at | 235146_at |
| | 206262_at | 218541_s_at | 235766_x_at |
| | 206377_at | 218546_at | 235849_at |
| | 206385_s_at | 219059_s_at | 238143_at |
| | 206664_at | 219543_at | 238750_at |
| | 207126_x_at | 219796_s_at | 238751_at |
| | 207245_at | 219948_x_at | 239272_at |
| | 207390_s_at | 220075_s_at | 241994_at |
| | 207392_x_at | 220266_s_at | 242447_at |
| | 207432_at | 220468_at | 242601_at |
| | 207761_s_at | 220645_at | 243278_at; and/or |
| (ii) | ADH1C | HIGD1A | NR3C2 |
| | AGR3 | HMGCS2 | P2RY1 |
| | ALDH1A1 | HPGD | PADI2 |
| | ANK3 | HSD11B2 | PAPSS2 |
| | ARL14 | HSD17B2 | PBLD |
| | ATP1A2 | HSPA2 | PDCD4 |
| | ATP8B1 | IGHA1 | PDE9A |
| | BEST2 | IGHM | PIGR |
| | C10orf99 | IL1R2 | PLCE1 |
| | C15orf48 | IL8 | PPID |
| | Clorf115 | IQGAP2 | PRKACB |
| | C4orf34 | ITLN1 | PTGER4 |
| | C6orf105 | ITM2C | RAB27A |
| | C8orf4 | KCNMA1 | SCARA5 |
| | CA12 | KIAA0828 | SDCBP2 |
| | CCL28 | KLF4 | SELENBP1 |
| | CKB | KRT20 | SI |
| | CLCA1 | LOC253012 | SMTN |
| | CLDN8 | LOC25845 | SORBS2 |
| | CLIC5 | LOC285382 | SRI |
| | CMBL | LOC572558 | SST |
| | CNTN3 | LOC646627 | ST6GALNAC1 |
| | DNASE1L3 | LOC652128 | SULT1A1 |
| | EDG2 | LOC96610 | TNXB |
| | ENAM | MAOA | TSPAN1 |
| | ENTPD5 | MATN2 | TTRAP |
| | ETHE1 | MEP1A | UGDH |
| | FABP1 | METTL7A | UGP2 |
| | FAM46C | MFSD4 | UGT1A1 |
| | FAM55D | MGC13057 | UGT1A3 |
| | FCGBP | MIER3 | UGT1A6 |
| | FGFR2 | MMP28 | UGT1A8 |
| | FOSB | MT1A | UGT1A9 |
| | FOXF2 | MT1F | UGT2A3 |
| | FOXP2 | MT1M | UGT2B15 |
| | FUCA1 | MUC12 | UGT2B17 |
| | GPA33 | MUC2 | VSIG2 |
| | HBA1 | MUC4 | XDH |
| | HBA2 | MUCDHL | XLKD1 |
| | HBB | MYH11 | ZCWPW2 |
| | HHLA2 | NDE1 | |

in a biological sample from said individual wherein a lower level of expression of the genes or transcripts of group (i) and/or group (ii) relative to control levels is indicative of a cancer cell
or a cell predisposed to the onset of a cancerous state.

According to these aspects, said control levels are preferably non-neoplastic levels and said large intestine tissue is colorectal tissue. Even more preferably, said biological sample is a stool sample or blood sample.

In one embodiment, said expression is assessed by screening for DNA changes which impact on methylation, in particular hypermethylation. In another embodiment, expression is assessed by the association of DNA with chromatin proteins carrying repressive modifications, for example, methylation of lysines 9 or 27 of histone H3.

In a related aspect, it has been determined that a subpopulation of the markers of the present invention are not only expressed at levels lower than normal levels, their expression pattern is uniquely characterized by the fact that expression levels above that of background control levels are not detectable in neoplastic tissue. This determination has therefore enabled the development of qualitative screening systems which are simply designed to detect marker expression relative to a control background level. In accordance with this aspect of the present invention, said “control level” is therefore the “background level”.

According to this aspect, there is therefore provided a method of screening for the onset or predisposition to the onset of a large intestine neoplasm in an individual, said method comprising screening the level of expression of one or more genes or transcripts selected from:

the gene, genes or transcripts detected by Affymetrix probeset IDs:

| | | | |
|---|---|---|---|
| (i) | 202920_at | 222717_at | 231120_x_at |
| | 203881_s_at | 224412_s_at | 231773_at |
| | 204719_at | 225381_at | 203296_s_at |
| | 204931_at | 225575_at | 206664_at |
| | 204940_at | 227529_s_at | 211549_s_at |
| | 205433_at | 227623_at | 214598_at |
| | 206637_at | 227705_at | 219948_x_at |
| | 207080_s_at | 227827_at | 220812_s_at |
| | 207980_s_at | 228504_at | 221305_s_at |
| | 209170_s_at | 228706_s_at | 229831_at |
| | 209209_s_at | 228766_at | 231925_at |
| | 209613_s_at | 228854_at | 235146_at |
| | 220037_s_at | 228885_at | 238751_at |
| | 220376_at | 230788_at | 243278_at; and/or |
| (ii) | ADH1B | ANGPTL1 | HHLA2 |
| | SORBS2 | DMD | SORBS2 |
| | PYY | GCNT2 | CLDN23 |
| | ABCA8 | SDPR | CNTN3 |
| | RPL24 | PKIB | PLEKHC1 |
| | SI | CITED2 | LRRC19 |
| | CLDN8 | TCF21 | LIFR |
| | P2RY14 | P2RY1 | ATP1A2 |
| | PLN | ANK2 | HPGD |
| | TRPM6 | XLKD1 | GPM6B |
| | CD36 | LOC399959 | UGT1A8 |
| | BCHE | AKAP12 | FOXP2 |
| | TCEAL7 | UGT2A3 | |

in a biological sample from said individual wherein a level of expression of the genes or transcripts of group (i) and/or group (ii) which is not substantially above background levels is indicative of a neoplastic cell or a cell predisposed to the onset of a neoplastic state.

In a most preferred embodiment, said genes or transcripts are selected from:
(i) the gene, genes or transcripts detected by Affymetrix probeset IDs: 209613_s_at, 227827_at, 204719_at, 228504_at, 228885_at, 206664_at, 207080_s_at; and/or
(ii) ADH1B, SORBS2, PYY, ABCA8, RPL24, SI Preferably, said neoplasm is an adenoma or an adenocarinoma and said gastrointestinal tissue is colorectal tissue.

In yet another embodiment, it has been determined that a further subpopulation of these markers are more characteristic of adenoma development, while others are more characteristic of cancer development. Accordingly, there is provided a convenient means of qualitatively obtaining indicative information in relation to the characteristics of the subject neoplasm.

According to this embodiment there is provided a method of screening for the onset or predisposition to the onset of a large intestine neoplasm in an individual, said method comprising screening the level of expression of one or more genes or transcripts selected from:
(i) the gene, genes or transcripts detected by Affymetrix probeset IDs: 209209_s_at, 225381_at, 227529_s_at 227623_at, 227705_at; and/or
(ii) AKAP12, LOC399959, PLEKHC1, TCEAL7
   in a biological sample from said individual wherein a lower level of expression of the genes or transcripts of group (i) and/or group (ii) which is not substantially above background levels is indicative of an adenoma cell or a cell predisposed to the onset of an adenoma state.

In yet still another preferred embodiment there is provided a method of screening for the onset or predisposition to the onset of a large intestine neoplasm in an individual, said method comprising screening the level of expression of one or more genes or transcripts selected from:

the gene, genes or transcripts detected by Affymetrix probeset IDs:

| | | | |
|---|---|---|---|
| (i) | 203296_s_at | 219948_x_at | 231925_at |
| | 206664_at | 220812_s_at | 235146_at |
| | 211549_s_at | 221305_s_at | 238751_at |
| | 214598_at | 229831_at | 243278_at; and/or |
| (ii) | ATP1A2 | HHLA2 | SORBS2 |
| | CLDN8 | HPGD | UGT1A8 |
| | CNTN3 | P2RY1 | UGT2A3 |
| | FOXP2 | SI | |

in a biological sample from said individual wherein a lower level of expression of the genes or transcripts of group (i) and/or group (ii) which is not substantially above background levels is indicative of a cancer cell or a cell predisposed to the onset of a cancerous state.

Preferably, said large intestine tissue is colorectal tissue.

More preferably, said biological sample is a stool sample or a blood sample.

In one embodiment, said expression is assessed by screening for DNA changes which impact on methylation, in particular hypermethylation. In another embodiment, expression is assessed by the association of DNA with chromatin proteins carrying repressive modifications, for example, methylation of lysines 9 or 27 of histone H3.

As detailed hereinbefore, the present invention is designed to screen for a neoplastic cell or cellular population, which is located in the large intestine. Accordingly, reference to “cell or cellular population” should be understood as a reference to an individual cell or a group of cells. Said group of cells may be a diffuse population of cells, a cell suspension, an encapsulated population of cells or a population of cells which take the form of tissue.

Reference to “expression” should be understood as a reference to the transcription and/or translation of a nucleic acid molecule. In this regard, the present invention is exemplified with respect to screening for neoplastic marker expression products taking the form of RNA transcripts (eg primary RNA or mRNA). Reference to “RNA” should be understood to encompass reference to any form of RNA, such as primary RNA or mRNA. Without limiting the present invention in any way, the modulation of gene transcription leading to increased or decreased RNA synthesis will also correlate with the translation of some of these RNA transcripts (such as mRNA) to produce a protein product. Accordingly, the present invention also extends to detection methodology which is directed to screening for modulated levels or patterns of the neoplastic marker protein products as an indicator of the neoplastic state of a cell or cellular population. Although one method is to screen for mRNA transcripts and/or the corresponding protein product, it should be understood that the present invention is not limited in this regard and extends to screening for any other form of neoplastic marker expression product such as, for example, a primary RNA transcript.

In terms of screening for the downregulation of expression of a marker it would also be well known to the person of skill in the art that changes which are detectable at the DNA level are indicative of changes to gene expression activity and therefore changes to expression product levels. Such changes include but are not limited to, changes to DNA methylation. Accordingly, reference herein to “screening the level of expression” and comparison of these “levels of expression” to control “levels of expression” should be understood as a reference to assessing DNA factors which are related to transcription, such as gene/DNA methylation patterns.

It would also be known to a person skilled in the art that changes in the structure of chromatin are indicative of changes in gene expression. Silencing of gene expression is often associated with modification of chromatin proteins, methylation of lysines at either or both positions 9 and 27 of histone H3 being well studied examples, while active chromatin is marked by acetylation of lysine 9 of histone H3. Thus association of gene sequences with chromatin carrying repressive or active modifications can be used to make an assessment of the expression level of a gene.

It is well within the skill of the person of skill in the art to determine the most appropriate screening method for any given situation. To this end, the genes which are known to encode an expression product which is either secreted by the cell or membrane bound is detailed in the table below. It would be appreciated that screening for neoplastic markers which are secreted or membrane bound may provide particular advantages in terms of the design of a diagnostic screening product.

The gene or genes detected by Asymetrix probe Nos:

| | | | |
|---|---|---|---|
| 200600_at | 205593_s_at | 212185_x_at | 224480_s_at |
| 200845_s_at | 205765_at | 212192_at | 224663_s_at |
| 200859_x_at | 205892_s_at | 212224_at | 224694_at |
| 200884_at | 205927_s_at | 212230_at | 224823_at |
| 200897_s_at | 205929_at | 212233_at | 224836_at |
| 200974_at | 205935_at | 212265_at | 224840_at |
| 201041_s_at | 205935_at | 212288_at | 224959_at |
| 201058_s_at | 205950_s_at | 212386_at | 224963_at |
| 201061_s_at | 206000_at | 212387_at | 224964_s_at |
| 201069_at | 206094_x_at | 212397_at | 224989_at |
| 201105_at | 206143_at | 212414_s_at | 224990_at |
| 201137_s_at | 206149_at | 212419_at | 225207_at |
| 201300_s_at | 206198_s_at | 212671_s_at | 225242_s_at |
| 201324_at | 206199_at | 212730_at | 225269_s_at |
| 201426_s_at | 206208_at | 212741_at | 225381_at |
| 201539_s_at | 206209_s_at | 212764_at | 225442_at |
| 201540_at | 206262_at | 212814_at | 225458_at |
| 201616_s_at | 206377_at | 212859_x_at | 225575_at |
| 201617_x_at | 206385_s_at | 212956_at | 225602_at |
| 201667_at | 206461_x_at | 213106_at | 225604_s_at |
| 201739_at | 206561_s_at | 213317_at | 225626_at |
| 201743_at | 206576_s_at | 213509_x_at | 225710_at |
| 201865_x_at | 206637_at | 213629_x_at | 225720_at |
| 201920_at | 206664_at | 213746_s_at | 225721_at |
| 201957_at | 206710_s_at | 213891_s_at | 225782_at |
| 202007_at | 206784_at | 213953_at | 225894_at |
| 202069_s_at | 207126_x_at | 214027_x_at | 225895_at |
| 202133_at | 207245_at | 214234_s_at | 226001_at |
| 202242_at | 207266_x_at | 214235_at | 226051_at |
| 202266_at | 207390_s_at | 214414_x_at | 226084_at |
| 202274_at | 207392_x_at | 214433_s_at | 226103_at |
| 202388_at | 207432_at | 214505_s_at | 226147_s_at |
| 202555_s_at | 207761_s_at | 214598_at | 226248_s_at |
| 202620_s_at | 207980_s_at | 214677_x_at | 226302_at |
| 202686_s_at | 208063_s_at | 214696_at | 226303_at |
| 202731_at | 208131_s_at | 214752_x_at | 226304_at |
| 202741_at | 208370_s_at | 214768_x_at | 226333_at |
| 202742_s_at | 208383_s_at | 214777_at | 226430_at |
| 202746_at | 208450_at | 215049_x_at | 226594_at |
| 202760_s_at | 208581_x_at | 215118_s_at | 226654_at |
| 202768_at | 208596_s_at | 215125_s_at | 226682_at |
| 202888_s_at | 208763_s_at | 215176_x_at | 226694_at |
| 202920_at | 208788_at | 215193_x_at | 226811_at |
| 202957_at | 208789_at | 215299_x_at | 226818_at |
| 202992_at | 208920_at | 215657_at | 226834_at |
| 202994_s_at | 208937_s_at | 216207_x_at | 226841_at |
| 202995_s_at | 209047_at | 216336_x_at | 227006_at |
| 203000_at | 209074_s_at | 216401_x_at | 227052_at |
| 203001_s_at | 209114_at | 216491_x_at | 227061_at |
| 203058_s_at | 209116_x_at | 216576_x_at | 227099_s_at |
| 203060_s_at | 209138_x_at | 216834_at | 227235_at |
| 203066_at | 209147_s_at | 216984_x_at | 227404_s_at |
| 203131_at | 209156_s_at | 217022_s_at | 227522_at |
| 203240_at | 209167_at | 217148_x_at | 227529_s_at |
| 203305_at | 209170_s_at | 217165_x_at | 227561_at |
| 203343_at | 209191_at | 217232_x_at | 227623_at |
| 203382_s_at | 209209_s_at | 217235_x_at | 227662_at |
| 203474_at | 209210_s_at | 217378_x_at | 227682_at |
| 203638_s_at | 209283_at | 217414_x_at | 227705_at |
| 203645_s_at | 209301_at | 217480_x_at | 227719_at |
| 203680_at | 209312_x_at | 217546_at | 227725_at |
| 203729_at | 209357_at | 217762_s_at | 227727_at |
| 203748_x_at | 209373_at | 217764_s_at | 227735_s_at |
| 203766_s_at | 209374_s_at | 217897_at | 227736_at |
| 203908_at | 209457_at | 217967_s_at | 228202_at |
| 203913_s_at | 209458_x_at | 218087_s_at | 228232_s_at |
| 203914_x_at | 209498_at | 218211_s_at | 228469_at |
| 203951_at | 209612_s_at | 218224_at | 228504_at |
| 203980_at | 209613_s_at | 218312_s_at | 228507_at |
| 204018_x_at | 209621_s_at | 218353_at | 228640_at |
| 204034_at | 209651_at | 218418_s_at | 228766_at |
| 204036_at | 209656_s_at | 218546_at | 228846_at |
| 204069_at | 209667_at | 218559_s_at | 228854_at |
| 204083_s_at | 209668_x_at | 219014_at | 228961_at |
| 204122_at | 209868_s_at | 219059_s_at | 229070_at |
| 204130_at | 209948_at | 219508_at | 229254_at |
| 204135_at | 210107_at | 219543_at | 229530_at |
| 204326_x_at | 210139_s_at | 219607_s_at | 229659_s_at |
| 204388_s_at | 210298_x_at | 219796_s_at | 229831_at |
| 204389_at | 210299_s_at | 219948_x_at | 229839_at |
| 204438_at | 210302_s_at | 219955_at | 230087_at |
| 204457_s_at | 210517_s_at | 220026_at | 230264_s_at |
| 204532_x_at | 210524_x_at | 220037_s_at | 230595_at |
| 204570_at | 210524_x_at | 220075_s_at | 230788_at |
| 204607_at | 210946_at | 220266_s_at | 230830_at |
| 204688_at | 211372_s_at | 220376_at | 231120_x_at |
| 204697_s_at | 211538_s_at | 220468_at | 231832_at |
| 204719_at | 211548_s_at | 220812_s_at | 231925_at |
| 204745_x_at | 211549_s_at | 220834_at | 231975_s_at |
| 204818_at | 211596_s_at | 221004_s_at | 232176_at |
| 204894_s_at | 211637_x_at | 221305_s_at | 232481_s_at |
| 204897_at | 211643_x_at | 221667_s_at | 233565_s_at |
| 204931_at | 211645_x_at | 221747_at | 234987_at |
| 204938_s_at | 211671_s_at | 221748_s_at | 235146_at |
| 204939_s_at | 211696_x_at | 221841_s_at | 235766_x_at |
| 204940_at | 211699_x_at | 221874_at | 235849_at |
| 204955_at | 211745_x_at | 221896_s_at | 235976_at |
| 205097_at | 211798_x_at | 222513_s_at | 236300_at |
| 205112_at | 211848_s_at | 222717_at | 236313_at |
| 205259_at | 211889_x_at | 223235_s_at | 236894_at |
| 205267_at | 211964_at | 223343_at | 237521_x_at |
| 205403_at | 211985_s_at | 223395_at | 238750_at |
| 205412_at | 211990_at | 223484_at | 241994_at |
| 205433_at | 211991_s_at | 223551_at | 242317_at |
| 205464_at | 212077_at | 223597_at | 242447_at |
| 205480_s_at | 212097_at | 223623_at | 242601_at |
| 205547_s_at | 212136_at | 224352_s_at | 243278_at |
| 205554_s_at | 212158_at | 224412_s_at | |
| ABCA8 | EGR1 | LOC25845 | PPID |
| ABI3BP | ELOVL5 | LOC283666 | PPP1R12B |
| ACAT1 | EMP1 | LOC285382 | PPP1R14A |
| ACTA2 | EMP3 | LOC339562 | PRDX6 |
| ACTG2 | ENTPD5 | LOC387763 | PRIMA1 |
| ADH1B | EPB41L3 | LOC399959 | PRKACB |
| ADH1C | ETHE1 | LOC572558 | PRKAR2B |
| AKAP12 | F13A1 | LOC63928 | PRNP |
| AKR1B10 | FABP1 | LOC652128 | PTGER4 |
| ALDH1A1 | FABP4 | LOC652745 | PTGIS |
| ANK2 | FAM107A | LRIG1 | PTRF |
| ANK3 | FAM129A | LRRC19 | QKI |
| ANKRD25 | FAM46C | MAB21L2 | RAB27A |
| ANPEP | FBLN1 | MAFB | RAB31 |
| ANTXR1 | FCGBP | MAGI | RBMS1 |
| AOC3 | FGFR2 | MALL | RDX |
| AP1S2 | FHL1 | MAOA | RELL1 |
| APOE | FILIP1L | MAP1B | RGS1 |
| AQP1 | FKBP5 | MEIS1 | RGS2 |
| AQP8 | FLNA | MEP1A | RGS5 |
| ARL14 | FNBP1 | METTL7A | SCARA5 |
| ATP2B4 | FOSB | MFSD4 | SCNN1B |
| ATP8A1 | FOXF1 | MGC14376 | SDC2 |
| AXL | FOXF2 | MIER3 | SDCBP2 |
| BCHE | FOXP2 | MLPH | SDPR |
| BEST2 | FXYD6 | MMP2 | SELENBP1 |
| C10orf56 | GALNAC4S- | MPEG1 | SELM |
| C10orf99 | 6ST | MPEG1 | SGCE |
| C15orf48 | GAS1 | MRC1 | SGK |
| C1orf115 | GCNT2 | MRGPRF | SI |
| C20orf118 | GCNT3 | MS4A12 | SLC20A1 |
| C2orf40 | GJA1 | MS4A4A | SLC26A2 |
| C4orf34 | GNG2 | MS4A7 | SLC26A3 |
| C6orf105 | GPA33 | MSN | SLC4A4 |
| C6orf204 | GPM6B | MSRB3 | SLITRK6 |
| C7 | GUCY1A3 | MT1A | SMOC2 |
| C9orf19 | HBA1 | MT1E | SMTN |
| CA1 | HBA2 | MT1F | SORBS1 |
| CA2 | HBB | MT1G | SRI |
| CA4 | HCLS1 | MT1H | SRPX |
| CALD1 | HHLA2 | MT1M | ST6GALNAC1 |
| CALM1 | HIGD1A | MT1X | STMN2 |
| CAPN9 | HLA-C | MT2A | STOM |
| CAV1 | HLA-DPA1 | MUC12 | SULT1A1 |
| CCDC80 | HLA-DPB1 | MUCDHL | SYNPO2 |
| CCL28 | HLA-DQA1 | MXD1 | TAGLN |
| CD14 | HLA-DRB1 | MYL9 | TBC1D9 |
| CD163 | HMGCS2 | MYLK | TCEAL7 |
| CD36 | HPGD | NEXN | TCF21 |
| CDKN2B | HSD11B2 | NID1 | TGFB1I1 |
| CEACAM1 | HSD17B2 | NR3C1 | TMEM47 |
| CEACAM7 | HSPA2 | NR3C2 | TNS1 |
| CES2 | HSPB6 | OSTbeta | TP53INP2 |
| CFL2 | HSPB8 | P2RY1 | TPM2 |
| CHGA | ID1 | P2RY14 | TRPM6 |
| CITED2 | IDH3A | PAG1 | TSC22D3 |
| CKB | IGHA1 | PALLD | TSPAN1 |
| CLCA1 | IGHM | PALM2- | TSPAN7 |
| CLCA4 | IGKV1D-13 | AKAP2 | TTRAP |
| CLDN8 | IGL@ | PAPSS2 | TUBB6 |
| CLIC5 | IGLJ3 | PBLD | TYROBP |
| CMBL | IL1R2 | PCK1 | UGDH |
| CNN1 | IQGAP2 | PDCD4 | UGP2 |
| CNTN3 | ITLN1 | PDE9A | UGT1A1 |
| COL4A2 | ITM2A | PDGFRA | UGT1A3 |
| COL6A2 | ITM2C | PDK4 | UGT1A6 |
| COX7A1 | KCNMA1 | PDLIM3 | UGT1A8 |
| CRYAB | KCNMB1 | PGM5 | UGT1A9 |
| CTSE | KCTD12 | PIGR | UGT2A3 |
| CYP3A5 | KIAA0828 | PKIB | UGT2B15 |
| CYP3A5P2 | KIAA1324 | PLAC8 | UGT2B17 |
| DDR2 | KLF4 | PLCE1 | VIM |
| DES | KLHL5 | PLEKHC1 | VSIG2 |
| DMN | KRT20 | PLN | WDR51B |
| DNASE1L3 | L1TD1 | PLOD2 | WWTR1 |
| DSCR1 | LGALS1 | PMP22 | XDH |
| DUSP1 | LGALS2 | PNMA1 | XLKD1 |
| DUSP5 | LIFR | POU2AF1 | ZSCAN18 |
| EDG2 | LMOD1 | PPAP2A | LOC253012 |
| PPAP2B | | | |

Reference to “nucleic acid molecule” should be understood as a reference to both deoxyribonucleic acid molecules and ribonucleic acid molecules and fragments thereof. The present invention therefore extends to both directly screening for mRNA levels in a biological sample or screening for the complementary cDNA which has been reverse-transcribed from an mRNA population of interest. It is well within the skill of the person of skill in the art to design methodology directed to screening for either DNA or RNA. As detailed above, the method of the present invention also extends to screening for the protein product translated from the subject mRNA or the genomic DNA itself.

In one preferred embodiment, the level of gene expression is measured by reference to genes which encode a protein product and, more particularly, said level of expression is measured at the protein level. Accordingly, to the extent that the present invention is directed to screening for markers which are detailed in the preceding table, said screening is preferably directed to the encoded protein.

In another particularly preferred embodiment, said gene expression is assessed by analysing genomic DNA methylation. In another embodiment, expression is assessed by the association of DNA with chromatin proteins carrying repressive modifications, for example, methylation of lysines 9 or 27 of histone H3.

As detailed hereinbefore, it should be understood that although the present invention is exemplified with respect to the detection of expressed nucleic acid molecules (e.g. mRNA), it also encompasses methods of detection based on screening for the protein product of the subject genes. The present invention should also be understood to encompass methods of detection based on identifying both proteins and/or nucleic acid molecules in one or more biological samples. This may be of particular significance to the extent that some of the neoplastic markers of interest may correspond to genes or gene fragments which do not encode a protein product. Accordingly, to the extent that this occurs it would not be possible to test for a protein and the subject marker would have to be assessed on the basis of transcription expression profiles or changes to genomic DNA.

The term “protein” should be understood to encompass peptides, polypeptides and proteins (including protein fragments). The protein may be glycosylated or unglycosylated and/or may contain a range of other molecules fused, linked, bound or otherwise associated to the protein such as amino acids, lipids, carbohydrates or other peptides, polypeptides or proteins.

Reference herein to a “protein” includes a protein comprising a sequence of amino acids as well as a protein associated with other molecules such as amino acids, lipids, carbohydrates or other peptides, polypeptides or proteins.

The proteins encoded by the neoplastic markers of the present invention may be in multimeric form meaning that two or more molecules are associated together. Where the same protein molecules are associated together, the complex is a homomultimer. An example of a homomultimer is a homodimer. Where at least one marker protein is associated with at least one non-marker protein, then the complex is a heteromultimer such as a heterodimer.

Reference to a “fragment” should be understood as a reference to a portion of the subject nucleic acid molecule or protein. This is particularly relevant with respect to screening for modulated RNA levels in stool samples since the subject RNA is likely to have been degraded or otherwise fragmented due to the environment of the gut. One may therefore actually be detecting fragments of the subject RNA molecule, which fragments are identified by virtue of the use of a suitably specific probe.

Reference to the “onset” of a neoplasm, such as adenoma or adenocarcinoma, should be understood as a reference to one or more cells of that individual exhibiting dysplasia. In this regard, the adenoma or adenocarcinoma may be well developed in that a mass of dysplastic cells has developed. Alternatively, the adenoma or adenocarcinoma may be at a very early stage in that only relatively few abnormal cell divisions have occurred at the time of diagnosis. The present invention also extends to the assessment of an individual's predisposition to the development of a neoplasm, such as an adenoma or adenocarcinoma. Without limiting the present invention in any way, changed levels of the neoplastic markers may be indicative of that individual's predisposition to developing a neoplasia, such as the future development of an adenoma or adenocarcinoma or another adenoma or adenocarcinoma.

In yet another related aspect of the present invention, markers have been identified which enable the characterisation of neoplastic tissue of the large intestine in terms of whether it is an adenoma or a cancer. This development now provides a simple yet accurate means of characterising tissue using means other than the traditional methods which are currently utilised.

According to this aspect of the present invention, there is provided a method of characterising a neoplastic cell or cellular population, which cell or cellular population is derived from the large intestine of an individual, said method comprising assessing the level of expression of one or more genes or transcripts selected from:

the gene, genes or transcripts detected by Affymetrix probeset IDs:

| | | | |
|---|---|---|---|
| (i) | 200600_at | 204006_s_at | 213428_s_at |
| | 200665_s_at | 204051_s_at | 213524_s_at |
| | 200832_s_at | 204122_at | 213869_x_at |
| | 200974_at | 204320_at | 213905_x_at |
| | 200986_at | 204475_at | 214247_s_at |
| | 201058_s_at | 204620_s_at | 215049_x_at |
| | 201069_at | 205479_s_at | 215076_s_at |
| | 201105_at | 205547_s_at | 215646_s_at |
| | 201141_at | 205828_at | 216442_x_at |
| | 201147_s_at | 207173_x_at | 217430_x_at |
| | 201150_s_at | 207191_s_at | 217762_s_at |
| | 201162_at | 208747_s_at | 217763_s_at |
| | 201163_s_at | 208782_at | 217764_s_at |
| | 201185_at | 208788_at | 218468_s_at |
| | 201261_x_at | 208850_s_at | 218469_at |
| | 201289_at | 208851_s_at | 218559_s_at |
| | 201426_s_at | 209101_at | 218638_s_at |
| | 201438_at | 209156_s_at | 219087_at |
| | 201616_s_at | 209218_at | 221011_s_at |
| | 201645_at | 209395_at | 221729_at |
| | 201667_at | 209396_s_at | 221730_at |
| | 201744_s_at | 209596_at | 221731_x_at |
| | 201792_at | 209875_s_at | 37892_at |
| | 201842_s_at | 209955_s_at | 223122_s_at |
| | 201852_x_at | 210095_s_at | 223235_s_at |
| | 201859_at | 210495_x_at | 224560_at |
| | 201893_x_at | 210511_s_at | 224694_at |
| | 202237_at | 210764_s_at | 224724_at |
| | 202238_s_at | 210809_s_at | 225664_at |
| | 202283_at | 211161_s_at | 225681_at |
| | 202291_s_at | 211571_s_at | 225710_at |
| | 202310_s_at | 211719_x_at | 225799_at |
| | 202311_s_at | 211813_x_at | 226237_at |
| | 202403_s_at | 211896_s_at | 226311_at |
| | 202404_s_at | 211959_at | 226694_at |
| | 202450_s_at | 211964_at | 226777_at |
| | 202620_s_at | 211966_at | 226930_at |
| | 202766_s_at | 211980_at | 227099_s_at |
| | 202859_x_at | 211981_at | 227140_at |
| | 202878_s_at | 212077_at | 227566_at |
| | 202917_s_at | 212344_at | 229218_at |
| | 202998_s_at | 212353_at | 229802_at |
| | 203083_at | 212354_at | 231579_s_at |
| | 203325_s_at | 212464_s_at | 231766_s_at |
| | 203382_s_at | 212488_at | 231879_at |
| | 203477_at | 212489_at | 232458_at |
| | 203570_at | 212667_at | 233555_s_at |
| | 203645_s_at | 213125_at | 234994_at; and/or |
| | 203878_s_at | | |
| (ii) | COL1A2 | LGALS1 | SRGN |
| | CTHRC1 | ELOVL5 | LBH |
| | FN1 | MGP | CTGF |
| | POSTN | MMP2 | TNC |
| | SPP1 | LOXL2 | G0S2 |
| | MMP1 | MYL9 | SQLE |
| | SPARC | DCN | EFEMP1 |
| | LUM | CALD1 | APOE |
| | GREM1 | FBN1 | MSN |
| | IL8 | MMP3 | IGFBP3 |
| | IGFBP5 | IGFBP7 | SERPINF1 |
| | SFRP2 | FSTL1 | ISLR |
| | SULF1 | COL4A2 | HNT |
| | ASPN | VCAN | COL5A1 |
| | COL6A3 | SMOC2 | OLFML2B |
| | COL8A1 | HTRA1 | KIAA1913 |
| | COL12A1 | CYR61 | PALM2-AKAP2 |
| | COL5A2 | FAP | SERPING1 |
| | CDH11 | VIM | TYROBP |
| | THBS2 | TIMP2 | ACTA2 |
| | COL15A1 | SCD | COL3A1 |
| | COL11A1 | TIMP3 | PLOD2 |
| | S100A8 | AEBP1 | MMP11 |
| | FNDC1 | GJA1 | CD163 |
| | SFRP4 | NNMT | FCGR3B |
| | INHBA | COL1A1 | PLAU |
| | COL6A2 | SULF2 | MAFB |
| | ANTXR1 | COL6A1 | LOC541471 |
| | GPNMB | SPON2 | LOC387763 |
| | BGN | CTSK | CHI3L1 |
| | TAGLN | MXRA5 | THY1 |
| | COL4A1 | C1S | LOXL1 |
| | RAB31 | DKK3 | CD93 |

in said cell or cellular population wherein a lower level of expression of the genes of group (i) and/or group (ii) relative to a gastrointestinal cancer control level is indicative of an adenoma cell or a cell predisposed to the onset of an adenoma state.

In another aspect there is provided a method of characterising a neoplastic cell or cellular population, which cell or cellular population is derived from the large intestine of an individual, said method comprising assessing the level of expression of one or more genes or transcripts selected from:

the gene, genes or transcripts detected by Affymetrix probeset IDs:

| | | | |
|---|---|---|---|
| (i) | 200884_at | 214234_s_at | 226248_s_at |
| | 203240_at | 214235_at | 226302_at |
| | 203963_at | 214433_s_at | 227676_at |
| | 204508_s_at | 215125_s_at | 227719_at |
| | 204607_at | 215867_x_at | 227725_at |
| | 204811_s_at | 217109_at | 228232_s_at |
| | 204895_x_at | 217110_s_at | 229070_at |
| | 204897_at | 218211_s_at | 231832_at |
| | 205259_at | 219543_at | 232176_at |
| | 205765_at | 219955_at | 232481_s_at |
| | 205927_s_at | 221841_s_at | 235976_at |
| | 208063_s_at | 221874_at | 236894_at |
| | 208937_s_at | 223969_s_at | 237521_x_at |
| | 210107_at | 223970_at | 242601_at; and/or |
| | 213106_at | | |
| (ii) | CLCA1 | CTSE | ATP8B1 |
| | FCGBP | C6orf105 | CACNA2D2 |
| | HMGCS2 | CKB | KLF4 |
| | RETNLB | ATP8A1 | CYP3A5P2 |
| | L1TD1 | MUC4 | CAPN9 |
| | SLITRK6 | UGT1A1 | NR3C2 |
| | VSIG2 | SELENBP1 | PBLD |
| | LOC253012 | PTGER4 | CA12 |
| | ST6GALNAC1 | MLPH | WDR51B |
| | ID1 | KIAA1324 | FAM3D |
| | CYP3A5 | | |

in a biological sample from said individual wherein a lower level of expression of the genes or transcripts of group (i) and/or group (ii) relative to a gastrointestinal adenoma control level is indicative of a cancer or a cell predisposed to the onset of a cancerous state.

Preferably, said gastrointestinal tissue is colorectal tissue.

In one embodiment, said expression is assessed by screening for DNA changes which impact on methylation, in particular hypermethylation. In another embodiment, expression is assessed by the association of DNA with chromatin proteins carrying repressive modifications, for example, methylation of lysines 9 or 27 of histone H3.

Reference to an “adenoma control level” or “cancer control level” should be understood as a reference to the level of said gene expression in a population of adenoma or cancer gastrointestinal cells, respectively. As discussed hereinbefore in relation to “normal levels”, the subject level may be a discrete level or a range of levels. Accordingly, the definition of “adenoma control level” or “cancer control level” should be understood to have a corresponding definition to “normal level”, albeit in the context of the expression of genes by a neoplastic population of large intestine cells.

In terms of this aspect of the present invention, the subject analysis is performed on a population of neoplastic cells. These cells may be derived in any manner, such as sloughed off neoplastic cells which have been collected via an enema wash or from a gastrointestinal sample, such as a stool sample. Alternatively, the subject cells may have been obtained via a biopsy or other surgical technique.

Without limiting this aspect of the invention in any way, several of the markers of this aspect of the present invention have been determined to be expressed at particularly significant levels below those of neoplastic cells. For example, decreased expression levels of 3 to 9 fold have been observed in respect of the following markers which are indicative of gastrointestinal adenomas, when assessed by the method herein exemplified.

| Fold | Gene, genes or transcripts | |
|---|---|---|
| Decrease | detected by Affymetrix Probe No: | Gene |
| 9 | 202404_s_at | COL1A2 |
| 8 | 225681_at | CTHRC1 |
| 7 | 212464_s_at | FN1 |
| | 210809_s_at | POSTN |
| 6 | 209875_s_at | SPP1 |
| 5 | 221740_at | MMP1 |
| | 204475_at | |
| 4 | 200665_s_at | SPARC |
| | 201744_s_at | LUM |
| | 218468_s_at | GREM1 |
| | 202859_x_at | IL8 |
| | 211959_at | IGFBP5 |
| 3 | 223122_s_at | SFRP2 |
| | 212353_at | SULF1 |
| | 219087_at | ASPN |
| | 201438_at | COL6A3 |
| | 226237_at | COL8A1 |
| | 225664_at | COL12A1 |
| | 221730_at | COL5A2 |
| | 207173_x_at | CDH11 |
| | 203083_at | THBS2 |
| | 203477_at | COL15A1 |
| | 37892_at | COL11A1 |
| | 202917_s_at | S100A8 |
| | 226930_at | FNDC1 |
| | 204051_s_at | SFRP4 |
| | 210511_s_at | INHBA |
| | 209156_s_at | COL6A2 |
| | 224694_at | ANTXR1 |
| | 201141_at | GPNMB |
| | 213905_x_at | BGN |
| | 205547_s_at | TAGLN |

In another example, decreased expression levels of between 3 to 5 fold have been observed in respect of the following markers which are indicative of gastrointestinal cancers, when assessed by the method herein exemplified.

| Fold | Gene, genes or transcripts | |
|---|---|---|
| Decrease | detected by Affymetrix Probe No: | Gene |
| 5 | 210107_at | CLCA1 |
| 3 | 203240_at | FCGBP |
| | 204607_at | HMGCS2 |
| | 223969_s_at | RETNLB |
| | 219955_at | L1TD1 |
| | 232481_s_at | SL1TRK6 |
| | 228232_s_at | VSIG2 |
| | 242601_at | LOC253012 |
| | 227725_at | ST6GALNAC1 |

According to this embodiment, there is therefore provided a method of characterising a neoplastic cell or cellular population, which cell or cellular population is derived from the large intestine of an individual, said method comprising assessing the level of expression of one or more genes or transcripts selected from:
(i) the gene or genes detected by Affymetrix probeset IDs: 202404_s_at, 212464_s_at, 210809_s_a, 225681_at; and/or
(ii) COL1A2, CTHRC1, FN1, POSTN
   in a biological sample from said individual wherein a lower level of expression of the genes or transcripts of group (i) and/or group (ii) relative to a gastrointestinal cancer control level is indicative of an adenoma cell or a cell predisposed to the onset of an adenoma state.

In another embodiment, there is provided a method of characterising a neoplastic cell or cellular population, which cell or cellular population is derived from the large intestine of an individual, said method comprising assessing the level of expression of one or more genes selected from:
(i) the gene, genes or transcripts detected by Affymetrix probeset IDs: 209875_s_at 227140_at 204475_at; and/or

the gene, genes or transcripts detected by Affymetrix probeset IDs:

| | | | |
|---|---|---|---|
| (i) | 209875_s_at | 227140_at | 204475_at; and/or |
| (ii) | SPP1 | MMP1 | |

in a biological sample from said individual wherein a lower level of expression of the genes or transcripts of group (i) and/or group (ii) relative to a gastrointestinal cancer control level is indicative of an adenoma cell or a cell predisposed to the onset of an adenoma state.

Preferably, said gastrointestinal tissue is colorectal tissue.

Still more preferably, said biological sample is a tissue sample.

In another preferred embodiment the present invention is directed to a method of characterising a cell or cellular population, which cell or cellular population is derived from the large intestine of an individual, said method comprising assessing the level of expression of one or more genes or transcripts selected from:

the gene, genes or transcripts detected by Affymetrix probeset IDs:

| | | | |
|---|---|---|---|
| (i) | 200665_s_at | 226237_at | 226930_at |
| | 201744_s_at | 225664_at | 204051_s_at |
| | 218468_s_at | 221730_at | 210511_s_at |
| | 202859_x_at | 207173_x_at | 209156_s_at |
| | 211959_at | 203083_at | 224694_at |
| | 223122_s_at | 203477_at | 201141_at |
| | 212353_at | 37892_at | 213905_x_at |
| | 219087_at | 202917_s_at | 205547_s_at |
| | 201438_at; and/or | | |
| (ii) | SPARC | COL8A1 | SFRP4 |
| | LUM | COL12A1 | INHBA |
| | GREM1 | COL5A2 | COL6A2 |
| | IL8 | CDH11 | ANTXR1 |
| | IGFBP5 | THBS2 | GPNMB |
| | SFRP2 | COL15A1 | BGN |
| | SULF1 | COL11A1 | TAGLN |
| | ASPN | S100A8 | |
| | COL6A3 | FNDC1 | |

in a biological sample from said individual wherein a lower level of expression of the genes or transcripts of group (i) and/or (ii) relative to a gastrointestinal cancer control level is indicative of an adenoma or a cell predisposed to the onset of an adenoma state.

In yet another preferred embodiment the present invention is directed to a method of characterising a cell or cellular population, which cell or cellular population is derived from the large intestine of an individual, said method comprising assessing the level of expression of one or more genes or transcripts selected from:
(i) the gene, genes or transcripts detected by Affymetrix probeset IDs: 210107 at; and/or
(ii) CLCA1
   in a biological sample from said individual wherein a lower level of expression of the genes or transcripts of group (i) and/or (ii) relative to a gastrointestinal adenoma control level is indicative of a cancer or a cell predisposed to the onset of a cancerous state.

In still yet another preferred embodiment the present invention is directed to a method of characterising a cell or cellular population, which cell or cellular population is derived from the large intestine of an individual, said method comprising assessing the level of expression of one or more genes or transcripts selected from:
(i) the gene, genes or transcripts detected by Affymetrix probeset IDs: 203240_at, 204607_at, 223969 s_at, 219955_at, 232481_s_at, 242601_at, 227725_at, 228232.s_s_at; and/or
(ii) FCGBP, HMGCS2, RETNLB, L1TD1, SLITRK6, VSIG2, LOC253012, ST6GALNAC1
   in a biological sample from said individual wherein a lower level of expression of the genes or transcripts of group (i) and/or (ii) relative to a gastrointestinal adenoma control level is indicative of a cancer or a cell predisposed to the onset of a cancerous state.

Preferably, said gastrointestinal tissue is colorectal tissue.

Even more preferably, said biological sample is a tissue sample.

In still another related aspect it has been determined that a subset of the markers of this aspect of the present invention are useful as qualitative markers of neoplastic tissue characterisation in that these markers, if not detectable at levels substantially above background levels in neoplastic tissue are indicative of cancerous tissue.

According to this aspect, the present invention provides a method of characterising a neoplastic cell or cellular population, which cell or cellular population is derived from the large intestine of an individual, said method comprising assessing the level of expression of one or more genes or transcripts selected from:

the gene or genes detected by Affymetrix probeset IDs:

| | | | |
|---|---|---|---|
| (i) | 235976_at | 236894_at | 237521; and/or |
| (ii) | SLURK6 | L1TD1 | |

in a biological sample from said individual wherein expression of the genes or transcripts of group (i) and/or (ii) at a level which is not substantially greater than background neoplastic tissue levels is indicative of a cancer or a cell predisposed to the onset of a cancerous state.

Preferably, said gastrointestinal tissue is colorectal tissue.

Still more preferably, said biological sample is a tissue sample.

In a most preferred embodiment, the methods of the present invention are preferably directed to screening for proteins encoded by the markers of the present invention or changes to DNA methylation of genomic DNA. In another embodiment, expression is assessed by the association of DNA with chromatin proteins carrying repressive modifications, for example, methylation of lysines 9 or 27 of histone H3.

Although the preferred method is to detect the expression product or DNA changes of the neoplastic markers for the purpose of diagnosing neoplasia development or predisposition thereto, the detection of converse changes in the levels of said markers may be desired under certain circumstances, for example, to monitor the effectiveness of therapeutic or prophylactic treatment directed to modulating a neoplastic condition, such as adenoma or adenocarcinoma development. For example, where reduced expression of the subject markers indicates that an individual has developed a condition characterised by adenoma or adenocarcinoma development, for example, screening for an increase in the levels of these markers subsequently to the onset of a therapeutic regime may be utilised to indicate reversal or other form of improvement of the subject individual's condition.

The method of the present invention is therefore useful as a one off test or as an on-going monitor of those individuals thought to be at risk of neoplasia development or as a monitor of the effectiveness of therapeutic or prophylactic treatment regimes directed to inhibiting or otherwise slowing neoplasia development. In these situations, mapping the modulation of neoplastic marker expression levels in any one or more classes of biological samples is a valuable indicator of the status of an individual or the effectiveness of a therapeutic or prophylactic regime which is currently in use. Accordingly, the method of the present invention should be understood to extend to monitoring for increases or decreases in marker expression levels in an individual relative to their normal level (as hereinbefore defined), background control levels, cancer levels, adenoma levels or relative to one or more earlier marker expression levels determined from a biological sample of said individual.

Means of assessing the subject expressed neoplasm markers in a biological sample can be achieved by any suitable method, which would be well known to the person of skill in the art. To this end, it would be appreciated that to the extent that one is examining either a homogeneous cellular population (such as a tumour biopsy or a cellular population which has been enriched from a heterogeneous starting population) or a tissue section, one may utilise a wide range of techniques such as in situ hybridisation, assessment of expression profiles by microassays, immunoassays and the like (hereinafter described in more detail) to detect the absence of or downregulation of the level of expression of one or more markers of interest. However, to the extent that one is screening a heterogenous cellular population or a bodily fluid in which heterogeneous populations of cells are found, such as a blood sample, the absence of or reduction in level of expression of a particular marker may be undetectable due to the inherent expression of the marker by non-neoplastic cells which are present in the sample. That is, a decrease in the level of expression of a subgroup of cells may not be detectable. In this situation, a more appropriate mechanism of detecting a reduction in a neoplastic subpopulation of the expression levels of one or more markers of the present invention is via indirect means, such as the detection of epigenetic changes.

Without limiting the present invention to any one theory or mode of action, during development gene expression is regulated by processes that alter the availability of genes for expression in different cell lineages without any alteration in gene sequence, and these states can be inherited through a cell division—a process called epigenetic inheritance. Epigenetic inheritance is determined by a combination of DNA methylation (modification of cytosine to give 5-methyl cytosine, 5 meC) and by modifications of the histone chromosomal proteins that package DNA. Thus methylation of DNA at CpG sites and modifications such as deacetylation of histone H3 on lysine 9, and methylation on lysine 9 or 27 are associated with inactive chromatin, while the converse state of a lack of DNA methylation, acetylation of lysine 9 of histone H3 is associated with open chromatin and active gene expression. In cancer, this epigenetic regulation of gene expression is frequently found to be disrupted (Esteller & Herman, 2000; Jones & Baylin, 2002). Genes such as tumour suppressor or metastasis suppressor genes are often found to be silenced by DNA methylation, while other genes may be hypomethylated and inappropriately expressed. Thus, among genes that show a decrease or loss of expression in cancer, this is often characterised by methylation of the promoter or regulatory region of the gene.

A variety of methods are available for detection of aberrantly methylated DNA of a specific gene, even in the presence of a large excess of normal DNA (Clark 2007). Thus, loss of expression of a gene which can be difficult to detect at the protein or RNA level except by immunohistochemistry can often be detected in tumour samples and in bodily fluids of cancer patients by the presence of hypermethylated DNA of the gene's promoter. Similarly DNA hypomethylation may be used for the detection of certain genes whose expression is elevated in cancer. Epigenetic alterations and chromatin changes in cancer are also evident in the altered association of modified histones with specific genes (Esteller, 2007); for example repressed genes are often found associated with histone H3 that is deacetylated and methylated on lysine 9. The use of antibodies targeted to altered histones allows for the isolation of DNA 0.15 associated with particular chromatin states and its potential use in cancer diagnosis.

Other methods of detecting changes to gene expression levels, particularly where the subject biological sample is not contaminated with high numbers of non-neoplastic cells, include but are not limited to:
(i) In vivo detection.
   Molecular Imaging may be used following administration of imaging probes or reagents capable of disclosing altered expression of the markers in the intestinal tissues.
   Molecular imaging (Moore et al., BBA, 1402:239-249, 1988; Weissleder et al., Nature Medicine 6:351-355, 2000) is the in vivo imaging of molecular expression that correlates with the macro-features currently visualized using “classical” diagnostic imaging techniques such as X-Ray, computed tomography (CT), MRI, Positron Emission Tomography (PET) or endoscopy.
(ii) Detection of downregulation of RNA expression in the cells by Fluorescent In Situ Hybridization (FISH), or in extracts from the cells by technologies such as Quantitative Reverse Transcriptase Polymerase Chain Reaction (QRTPCR) or Flow cytometric qualification of competitive RT-PCR products (Wedemeyer et al, Clinical Chemistry 48:9 1398-1405, 2002).
(iii) Assessment of expression profiles of RNA, for example by array technologies (Alon et al., Proc. Natl. Acad Sci. USA: 96, 6745-6750, June 1999).

A “microarray” is a linear or multi-dimensional array of preferably discrete tlgions, each having a defined area, formed on the surface of a solid support. The density of the discrete regions on a microarray is determined by the total numbers of target polynucleotides to be detected on the surface of a single solid phase support. As used herein, a DNA microarray is an array of oligonucleotide probes placed onto a chip or other surfaces used to amplify or clone target polynucleotides. Since the position of each particular group of probes in the array is known, the identities of the target polynucleotides can be determined based on their binding to a particular position in the microarray.

Recent developments in DNA microarray technology make it possible to conduct a large scale assay of a plurality of target nucleic acid molecules on a single solid phase support. U.S. Pat. No. 5,837,832 (Chee at al.) and related patent applications describe immobilizing an array of oligonucleotide probes for hybridization and detection of specific nucleic acid sequences in a sample. Target polynucleotides of interest isolated from a tissue of interest are hybridized to the DNA chip and the specific sequences detected based on the target polynucleotides' preference and degree of hybridization at discrete probe locations. One important use of arrays is in the analysis of differential gene expression, where the profile of expression of genes in different cells or tissues, often a tissue of interest and a control tissue, is compared and any differences in gene expression among the respective tissues are identified. Such information is useful for the identification of the types of genes expressed in a particular tissue type and diagnosis of conditions based on the expression profile.

In one example, RNA from the sample of interest is subjected to reverse transcription to obtain labelled cDNA. See U.S. Pat. No. 6,410,229 (Lockhart at al.) The cDNA is then hybridized to oligonucleotides or cDNAs of known sequence arrayed on a chip or other surface in a known order. In another example, the RNA is isolated from a biological sample and hybridised to a chip on which are anchored cDNA probes. The location of the oligonucleotide to which the labelled cDNA hybridizes provides sequence information on the cDNA, while the amount of labelled hybridized RNA or cDNA provides an estimate of the relative representation of the RNA or cDNA of interest. See Schena, et al. Science 270:467-470 (1995). For example, use of a cDNA microarray to analyze gene expression patterns in human cancer is described by DeRisi, et al. (Nature Genetics 14:457-460 (1996)).

In a preferred embodiment, nucleic acid probes corresponding to the subject nucleic acids are made. The nucleic acid probes attached to the biochip are designed to be substantially complementary to the nucleic acids of the biological sample such that specific hybridization of the target sequence and the probes of the present invention occurs. This complementarity need not be perfect, in that there may be any number of base pair mismatches that will interfere with hybridization between the target sequence and the single stranded nucleic acids of the present invention. It is expected that the overall homology of the genes at the nucleotide level probably will be about 40% or greater, probably about 60% or greater, and even more probably about 80% or greater, and in addition that there will be corresponding contiguous sequences of about 8-12 nucleotides or longer. However, if the number of mutations is so great that no hybridization can occur under even the least stringent of hybridization conditions, the sequence is not a complementary target sequence. Thus, by “substantially complementary” herein is meant that the probes are sufficiently complementary to the target sequences to hybridize under normal reaction conditions, particularly high stringency conditions.

A nucleic acid probe is generally single stranded but can be partly single and partly double stranded. The strandedness of the probe is dictated by the structure, composition, and properties of the target sequence. In general, the oligonucleotide probes range from about 6, 8, 10, 12, 15, 20, 30 to about 100 bases long, with from about 10 to about 80 bases being preferred, and from about 15 to about 40 bases being particularly preferred. That is, generally entire genes are rarely used as probes. In some embodiments, much longer nucleic acids can be used, up to hundreds of bases. The probes are sufficiently specific to hybridize to a complementary template sequence under conditions known by those of skill in the art. The number of mismatches between the probe's sequences and their complementary template (target) sequences to which they hybridize during hybridization generally do not exceed 15%, usually do not exceed 10% and preferably do not exceed 5%, as-determined by BLAST (default settings).

Oligonucleotide probes can include the naturally-occurring heterocyclic bases normally found in nucleic acids (uracil, cytosine, thymine, adenine and guanine), as well as modified bases and base analogues. Any modified base or base analogue compatible with hybridization of the probe to a target sequence is useful in the practice of the invention. The sugar or glycoside portion of the probe can comprise deoxyribose, ribose, and/or modified forms of these sugars, such as, for example, 2′-O-alkyl ribose. In a preferred embodiment, the sugar moiety is 2′-deoxyribose; however, any sugar moiety that is compatible with the ability of the probe to hybridize to a target sequence can be used.

In one embodiment, the nucleoside units of the probe are linked by a phosphodiester backbone, as is well known in the art. In additional embodiments, internucleotide linkages can include any linkage known to one of skill in the art that is compatible with specific hybridization of the probe including, but not limited to phosphorothioate, methylphosphonate, sulfamate (e.g., U.S. Pat. No. 5,470,967) and polyamide (i.e., peptide nucleic acids). Peptide nucleic acids are described in Nielsen et al. (1991) Science 254: 1497-1500, U.S. Pat. No. 5,714,331, and Nielsen (1999) Curr. Opin. Biotechnol. 10:71-75.

In certain embodiments, the probe can be a chimeric molecule; i.e., can comprise more than one type of base or sugar subunit, and/or the linkages can be of more than one type within the same primer. The probe can comprise a moiety to facilitate hybridization to its target sequence, as are known in the art, for example, intercalators and/or minor groove binders. Variations of the bases, sugars, and internucleoside backbone, as well as the presence of any pendant group on the probe, will be compatible with the ability of the probe to bind, in a sequence-specific fashion, with its target sequence. A large number of structural modifications, are possible within these bounds. Advantageously, the probes according to the present invention may have structural characteristics such that they allow the signal amplification, such structural characteristics being, for example, branched DNA probes as those described by Urdea et al. (Nucleic Acids Symp. Ser., 24:197-200 (1991)) or in the European Patent No. EP-0225,807. Moreover, synthetic methods for preparing the various heterocyclic bases, sugars, nucleosides and nucleotides that form the probe, and preparation of oligonucleotides of specific predetermined sequence, are well-developed and known in the art. A preferred method for oligonucleotide synthesis incorporates the teaching of U.S. Pat. No. 5,419,966.

Multiple probes may be designed for a particular target nucleic acid to account for polymorphism and/or secondary structure in the target nucleic acid, redundancy of data and the like. In some embodiments, where more than one probe per sequence is used, either overlapping probes or probes to different sections of a single target gene are used. That is, two, three, four or more probes, are used to build in a redundancy for a particular target. The probes can be overlapping (i.e. have some sequence in common), or are specific for distinct sequences of a gene. When multiple target polynucleotides are to be detected according to the present invention, each probe or probe group corresponding to a particular target polynucleotide is situated in a discrete area of the microarray.

Probes may be in solution, such as in wells or on the surface of a micro-array, or attached to a solid support. Examples of solid support materials that can be used include a plastic, a ceramic, a metal, a resin, a gel and a membrane. Useful types of solid supports include plates, beads, magnetic material, microbeads, hybridization chips, membranes, crystals, ceramics and self-assembling monolayers. One example comprises a two-dimensional or three-dimensional matrix, such as a gel or hybridization chip with multiple probe binding sites (Pevzner at al, J. Biomol. Struc. & Dyn. 9:399-410, 1991; Maskos and Southern, Nuc. Acids Res. 20:1679-84, 1992). Hybridization chips can be used to construct very large probe arrays that are subsequently hybridized with a target nucleic acid. Analysis of the hybridization pattern of the chip can assist in the identification of the target nucleotide sequence. Patterns can be manually or computer analyzed, but it is clear that positional sequencing by hybridization lends itself to computer analysis and automation. In another example, one may use an Affymetrix chip on a solid phase structural support in combination with a fluorescent bead based approach. In yet another example, one may utilise a cDNA microarray. In this regard, the oligonucleotides described by Lockkart et al (i.e. Affymetrix synthesis probes in situ on the solid phase) are particularly preferred, that is, photolithography.

As will be appreciated by those in the art, nucleic acids can be attached or immobilized to a solid support in a wide variety of ways. By “immobilized” herein is meant the association or binding between the nucleic acid probe and the solid support is sufficient to be stable under the conditions of binding, washing, analysis, and removal. The binding can be covalent or non-covalent. By “non-covalent binding” and grammatical equivalents herein is meant one or more of either electrostatic, hydrophilic, and hydrophobic interactions. Included in non-covalent binding is the covalent attachment of a molecule, such as streptavidin, to the support and the non-covalent binding of the biotinylated probe to the streptavidin. By “covalent binding” and grammatical equivalents herein is meant that the two moieties, the solid support and the probe, are attached by at least one bond, including sigma bonds, pi bonds and coordination bonds. Covalent bonds can be formed directly between the probe and the solid support or can be formed by a cross linker or by inclusion of a specific reactive group on either the solid support or the probe or both molecules. Immobilization may also involve a combination of covalent and non-covalent interactions.

Nucleic acid probes may be attached to the solid support by covalent binding such as by conjugation with a coupling agent or by covalent or non-covalent binding such as electrostatic interactions, hydrogen bonds or antibody-antigen coupling, or by combinations thereof. Typical coupling agents include biotin/avidin, biotin/streptavidin, Staphylococcus aureus protein A/IgG antibody P, fragment, and streptavidin/protein A chimeras (T. Sano and C. R. Cantor, Bio/Technology 9:1378-81 (1991)), or derivatives or combinations of these agents. Nucleic acids may be attached to the solid support by a photocleavable bond, an electrostatic bond, a disulfide bond, a peptide bond, a diester bond or a combination of these sorts of bonds. The array may also be attached to the solid support by a selectively releasable bond such as 4,4′-dimethoxytrityl or its derivative. Derivatives which have been found to be useful include 3 or 4 [bis-(4-methoxyphenyl)]-methyl-benzoic acid, N-succinimidyl-3 or 4 [bis-(4-methoxyphenyl)]-methyl-benzoic acid, N-succinimidyl-3 or 4 [bis-(4-methoxyphenyl)]-hydroxymethyl-benzoic acid, N-succinimidyl-3 or 4 [bis-(4-methoxyphenyl)]-chloromethyl-benzoic acid, and salts of these acids.

In general, the probes are attached to the biochip in a wide variety of ways, as will be appreciated by those in the art. As described herein, the nucleic acids can either be synthesized first, with subsequent attachment to the biochip, or can be directly synthesized on the biochip.

The biochip comprises a suitable solid substrate. By “substrate” or “solid support” or other grammatical equivalents herein is meant any material that can be modified to contain discrete individual sites appropriate for the attachment or association of the nucleic acid probes and is amenable to at least one detection method. The solid phase support of the present invention can be of any solid materials and structures suitable for supporting nucleotide hybridization and synthesis. Preferably, the solid phase support comprises at least one substantially rigid surface on which the primers can be immobilized and the reverse transcriptase reaction performed. The substrates with which the polynucleotide microarray elements are stably associated and may be fabricated from a variety of materials, including plastics, ceramics, metals, acrylamide, cellulose, nitrocellulose, glass, polystyrene, polyethylene vinyl acetate, polypropylene, polymethacrylate, polyethylene, polyethylene oxide, polysilicates, polycarbonates, Teflon, fluorocarbons, nylon, silicon rubber, polyanhydrides, polyglycolic acid, polylactic acid, polyorthoesters, polypropylfumerate, collagen, glycosaminoglycans, and polyamino acids. Substrates may be two-dimensional or three-dimensional in form, such as gels, membranes, thin films, glasses, plates, cylinders, beads, magnetic beads, optical fibers, woven fibers, etc. A preferred form of array is a three-dimensional array. A preferred three-dimensional array is a collection of tagged beads. Each tagged bead has different primers attached to it Tags are detectable by signalling means such as color (Luminex, Illumina) and electromagnetic field (Pharmaseq) and signals on tagged beads can even be remotely detected (e.g., using optical fibers). The size of the solid support can be any of the standard microarray sizes, useful for DNA microarray technology, and the size may be tailored to fit the particular machine being used to conduct a reaction of the invention. In general, the substrates allow optical detection and do not appreciably fluoresce.

In one embodiment, the surface of the biochip and the probe may be derivatized with chemical functional groups for subsequent attachment of the two. Thus, for example, the biochip is derivatized with a chemical functional group including, but not limited to, amino groups, carboxy groups, oxo groups and thiol groups, with amino groups being particularly preferred. Using these functional groups, the probes can be attached using functional groups on the probes. For example, nucleic acids containing amino groups can be attached to surfaces comprising amino groups, for example using linkers as are known in the art; for example, homo- or hetero-bifunctional linkers as are well known. In addition, in some cases, additional linkers, such as alkyl groups (including substituted and heteroalkyl groups) may be used.

In this embodiment, the oligonucleotides are synthesized as is known in the art, and then attached to the surface of the solid support. As will be appreciated by those skilled in the art, either the 5′ or 3′ terminus may be attached to the solid support, or attachment may be via an internal nucleoside. In an additional embodiment, the immobilization to the solid support may be very strong, yet non-covalent. For example, biotinylated oligonucleotides can be made, which bind to surfaces covalently coated with streptavidin, resulting in attachment.

The arrays may be produced according to any convenient methodology, such as preforming the polynucleotide microarray elements and then stably associating them with the surface. Alternatively, the oligonucleotides may be synthesized on the surface, as is known in the art. A number of different array configurations and methods for their production are known to those of skill in the art and disclosed in WO 95/25116 and WO 95/35505 (photolithographic techniques), U.S. Pat. No. 5,445,934 (in situ synthesis by photolithography), U.S. Pat. No. 5,384,261 (in situ synthesis by mechanically directed flow paths); and U.S. Pat. No. 5,700,637 (synthesis by spotting, printing or coupling); the disclosure of which are herein incorporated in their entirety by reference. Another method for coupling DNA to beads uses specific ligands attached to the end of the DNA to link to ligand-binding molecules attached to a bead. Possible ligand-binding partner pairs include biotin-avidin/streptavidin, or various antibody/antigen pairs such as digoxygenin-antidigoxygenin antibody (Smith et al., Science 258:1122-1126 (1992)). Covalent chemical attachment of DNA to the support can be accomplished by using standard coupling agents to link the 5′-phosphate on the DNA to coated microspheres through a phosphoamidate bond. Methods for immobilization of oligonucleotides to solid-state substrates are well established. See Pease et al., Proc. Natl. Acad. Sci. USA 91(11):5022-5026 (1994). A preferred method of attaching oligonucleotides to solid-state substrates is described by Guo at al., Nucleic Acids Res. 22:5456-5465 (1994). Immobilization can be accomplished either by in situ DNA synthesis (Maskos and Southern, supra) or by covalent attachment of chemically synthesized oligonucleotides (Guo at al., supra) in combination with robotic arraying technologies.

In addition to the solid-phase technology represented by biochip arrays, gene expression can also be quantified using liquid-phase arrays. One such system is kinetic polymerase chain reaction (PCR). Kinetic PCR allows for the simultaneous amplification and quantification of specific nucleic acid sequences. The specificity is derived from synthetic oligonucleotide primers designed to preferentially adhere to single-stranded nucleic acid sequences bracketing the target site. This pair of oligonucleotide primers form specific, non-covalently bound complexes on each strand of the target sequence. These complexes facilitate in vitro transcription of double-stranded DNA in opposite orientations. Temperature cycling of the reaction mixture creates a continuous cycle of primer binding, transcription, and re-melting of the nucleic acid to individual strands. The result is an exponential increase of the target dsDNA product. This product can be quantified in real time either through the use of an intercalating dye or a sequence specific probe. SYBR(r) Green 1, is an example of an intercalating dye, that preferentially binds to dsDNA resulting in a concomitant increase in the fluorescent signal. Sequence specific probes, such as used with TaqMan technology, consist of a fluorochrome and a quenching molecule covalently bound to opposite ends of an oligonucleotide. The probe is designed to selectively bind the target DNA sequence between the two primers. When the DNA strands are synthesized during the PCR reaction, the fluorochrome is cleaved from the probe by the exonuclease activity of the polymerase resulting in signal dequenching. The probe signalling method can be more specific than the intercalating dye method, but in each case, signal strength is proportional to the dsDNA product produced. Each type of quantification method can be used in multi-well liquid phase arrays with each well representing primers and/or probes specific to nucleic acid sequences of interest. When used with messenger RNA preparations of tissues or cell lines, an array of probe/primer reactions can simultaneously quantify the expression of multiple gene products of interest. See Germer et al., Genome Res. 10:258-266 (2000); Heid at al., Genome Res. 6:986-994 (1996).
(iv) Measurement of altered neoplastic marker protein levels in cell extracts, for example by immunoassay.

Testing for proteinaceous neoplastic marker expression product in a biological sample can be performed by any one of a number of suitable methods which are well known to those skilled in the art. Examples of suitable methods include, but are not limited to, antibody screening of tissue sections, biopsy specimens or bodily fluid samples.

To the extent that antibody based methods of diagnosis are used, the presence of the marker protein may be determined in a number of ways such as by Western blotting, ELISA or flow cytometry procedures. These, of course, include both single-site and two-site or “sandwich” assays of the non-competitive types, as well as in the traditional competitive binding assays. These assays also include direct binding of a labelled antibody to a target.

Sandwich assays are a useful and commonly used assay. A number of variations of the sandwich assay technique exist, and all are intended to be encompassed by the present invention. Briefly, in a typical forward assay, an unlabelled antibody is immobilized on a solid substrate and the sample to be tested brought into contact with the bound molecule. After a suitable period of incubation, for a period of time sufficient to allow formation of an antibody-antigen complex, a second antibody specific to the antigen, labelled with a reporter molecule capable of producing a detectable signal is then added and incubated, allowing time sufficient for the formation of another complex of antibody-antigen-labelled antibody. Any unreacted material is washed away, and the presence of the antigen is determined by observation of a signal produced by the reporter molecule. The results may either be qualitative, by simple observation of the visible signal, or may be quantitated by comparing with a control sample. Variations on the forward assay include a simultaneous assay, in which both sample and labelled antibody are added simultaneously to the bound antibody. These techniques are well known to those skilled in the art, including any minor variations as will be readily apparent.

In the typical forward sandwich assay, a first antibody having specificity for the marker or antigenic parts thereof; is either covalently or passively bound to a solid surface. The solid surface is typically glass or a polymer, the most commonly used polymers being cellulose, polyacrylamide, nylon, polystyrene, polyvinyl chloride or polypropylene. The solid supports may be in the form of tubes, beads, discs of microplates, or any other surface suitable for conducting an immunoassay. The binding processes are well-known in the art and generally consist of cross-linking, covalently binding or physically adsorbing, the polymer-antibody complex is washed in preparation for the test sample. An aliquot of the sample to be tested is then added to the solid phase complex and incubated for a period of time sufficient (e.g. 2-40 minutes) and under suitable conditions (e.g. 25′C) to allow binding of any subunit present in the antibody. Following the incubation period, the antibody subunit solid phase is washed and dried and incubated with a second antibody specific for a portion of the antigen. The second antibody is linked to a reporter molecule which is used to indicate the binding of the second antibody to the antigen.

An alternative method involves immobilizing the target molecules in the biological sample and then exposing the immobilized target to specific antibody which may or may not be labelled with a reporter molecule. Depending on the amount of target and the strength of the reporter molecule signal, a bound target may be detectable by direct labelling with the antibody. Alternatively, a second labelled antibody, specific to the first antibody is exposed to the target-first antibody complex to form a target-first antibody-second antibody tertiary complex. The complex is detected by the signal emitted by the reporter molecule.

By “reporter molecule” as used in the present specification, is meant a molecule which, by its chemical nature, provides an analytically identifiable signal which allows the detection of antigen-bound antibody. Detection may be either qualitative or quantitative. The most commonly used reporter molecules in this type of assay are either enzymes, fluorophores or radionuclide containing molecules (i.e. radioisotopes) and chemiluminescent molecules.

In the case of an enzyme immunoassay, an enzyme is conjugated to the second antibody, generally by means of glutaraldehyde or periodate. As will be readily recognized, however, a wide variety of different conjugation techniques exist, which are readily available to the skilled artisan. Commonly used enzymes include horseradish peroxidase, glucose oxidase, beta-galactosidase and alkaline phosphatase, amongst others. The substrates to be used with the specific enzymes are generally chosen for the production, upon hydrolysis by the corresponding enzyme, of a detectable color change. Examples of suitable enzymes include alkaline phosphatase and peroxidase. It is also possible to employ fluorogenic substrates, which yield a fluorescent product rather than the chromogenic substrates noted above. In all cases, the enzyme-labelled antibody is added to the first antibody hapten complex, allowed to bind, and then the excess reagent is washed away. A solution containing the appropriate substrate is then added to the complex of antibody-antigen-antibody. The substrate will react with the enzyme linked to the second antibody, giving a qualitative visual signal, which may be further quantitated, usually spectrophotometrically, to give an indication of the amount of antigen which was present in the sample. “Reporter molecule” also extends to use of cell agglutination or inhibition of agglutination such as red blood cells on latex beads, and the like.

Alternately, fluorescent compounds, such as fluorecein and rhodamine, may be chemically coupled to antibodies without altering their binding capacity. When activated by illumination with light of a particular wavelength, the fluorochrome-labelled antibody adsorbs the light energy, inducing a state to excitability in the molecule, followed by emission of the light at a characteristic color visually detectable with a light microscope. As in the EIA, the fluorescent labelled antibody is allowed to bind to the first antibody-hapten complex. After washing off the unbound reagent, the remaining tertiary complex is then exposed to the light of the appropriate wavelength the fluorescence observed indicates the presence of the hapten of interest. Immunofluorescence and EIA techniques are both very well established in the art and are particularly preferred for the present method. However, other reporter molecules, such as radioisotope, chemiluminescent or bioluminescent molecules, may also be employed.
(v) Determining altered expression of protein neoplastic markers on the cell surface, for example by immunohistochemistry.
(vi) Determining altered protein expression based on any suitable functional test, enzymatic test or immunological test in addition to those detailed in points (iv) and (v) above.

A person of ordinary skill in the art could determine, as a matter of routine procedure, the appropriateness of applying a given method to a particular type of biological sample.

Without limiting the present invention in any way, and as detailed above, gene expression levels can be measured by a variety of methods known in the art. For example, gene transcription or translation products can be measured. Gene transcription products, i.e., RNA, can be measured, for example, by hybridization assays, run-off assays., Northern blots, or other methods known in the art.

Hybridization assays generally involve the use of oligonucleotide probes that hybridize to the single-stranded RNA transcription products. Thus, the oligonucleotide probes are complementary to the transcribed RNA expression product. Typically, a sequence-specific probe can be directed to hybridize to RNA or cDNA. A “nucleic acid probe”, as used herein, can be a DNA probe or an RNA probe that hybridizes to a complementary sequence. One of skill in the art would know how to design such a probe such that sequence specific hybridization will occur. One of skill in the art will further know how to quantify the amount of sequence specific hybridization as a measure of the amount of gene expression for the gene was transcribed to produce the specific RNA.

The hybridization sample is maintained under conditions that are sufficient to allow specific hybridization of the nucleic acid probe to a specific gene expression product. “Specific hybridization”, as used herein, indicates near exact hybridization (e.g., with few if any mismatches). Specific hybridization can be performed under high stringency conditions or moderate stringency conditions. In one embodiment, the hybridization conditions for specific hybridization are high stringency. For example, certain high stringency conditions can be used to distinguish perfectly complementary nucleic acids from those of less complementarity. “High stringency conditions”, “moderate stringency conditions” and “low stringency conditions” for nucleic acid hybridizations are explained on pages 2.10.1-2.10.16 and pages 6.3.1-6.3.6 in Current Protocols in Molecular Biology (Ausubel, F. et al., “Current Protocols in Molecular Biology”, John Wiley & Sons, (1998), the entire teachings of which are incorporated by reference herein). The exact conditions that determine the stringency of hybridization depend not only on ionic strength (e.g., 0.2.times.SSC, 0.1.times.SSC), temperature (e.g., room temperature, 42° C., 68° C.) and the concentration of destabilizing agents such as formamide or denaturing agents such as SDS, but also on factors such as the length of the nucleic acid sequence, base composition, percent mismatch between hybridizing sequences and the frequency of occurrence of subsets of that sequence within other non-identical sequences. Thus, equivalent conditions can be determined by varying one or more of these parameters while maintaining a similar degree of identity or similarity between the two nucleic acid molecules. Typically, conditions are used such that sequences at least about 60%, at least about 70%, at least about 80%, at least about 90% or at least about 95% or more identical to each other remain hybridized to one another. By varying hybridization conditions from a level of stringency at which no hybridization occurs to a level at which hybridization is first observed, conditions that will allow a given sequence to hybridize (e.g., selectively) with the most complementary sequences in the sample can be determined.

Exemplary conditions that describe the determination of wash conditions for moderate or low stringency conditions are described in Kraus, M. and Aaronson, S., 1991. Methods Enzymol., 200:546-556; and in, Ausubel et al., Current Protocols in Molecular Biology, John Wiley & Sons, (1998). Washing is the step in which conditions are usually set so as to determine a minimum level of complementarity of the hybrids. Generally, starting from the lowest temperature at which only homologous hybridization occurs, each ° C. by which the final wash temperature is reduced (holding SSC concentration constant) allows an increase by 1% in the maximum mismatch percentage among the sequences that hybridize. Generally, doubling the concentration of SSC results in an increase in Tₘ of about 17° C. Using these guidelines, the wash temperature can be determined empirically for high, moderate or low stringency, depending on the level of mismatch sought. For example, a low stringency wash can comprise washing in a solution containing 0.2.times.SSC/0.1% SDS for 10 minutes at room temperature; a moderate stringency wash can comprise washing in a pre-warmed solution (42° C.) solution containing 0.2.times.SSC/0.1% SDS for 15 minutes at 42° C.; and a high stringency wash can comprise washing in pre-warmed (68° C.) solution containing 0.1.times.SSC/0.1% SDS for 15 minutes at 68° C. Furthermore, washes can be performed repeatedly or sequentially to obtain a desired result as known in the art. Equivalent conditions can be determined by varying one or more of the parameters given as an example, as known in the art, while maintaining a similar degree of complementarity between the target nucleic acid molecule and the primer or probe used (e.g., the sequence to be hybridized).

A related aspect of the present invention provides a molecular array, which array comprises a plurality of
(i) nucleic acid molecules comprising a nucleotide sequence corresponding to any one or more of the neoplastic marker genes hereinbefore described or a sequence exhibiting at least 80% identity thereto or a functional derivative, fragment, variant or homologue of said nucleic acid molecule; or
(ii) nucleic acid molecules comprising a nucleotide sequence capable of hybridising to any one or more of the sequences of (i) under medium stringency conditions or a functional derivative, fragment, variant or homologue of said nucleic acid molecule; or
(iii) nucleic acid probes or oligonucleotides comprising a nucleotide sequence capable of hybridising to any one or more of the sequences of (i) under medium stringency conditions or a functional derivative, fragment, variant or homologue of said nucleic acid molecule; or
(iv) probes capable of binding to any one or more of the proteins encoded by the nucleic acid molecules of (i) or a derivative, fragment or, homologue thereof

wherein the level of expression of said marker genes of (i) or proteins of (iv) is indicative of the neoplastic state of a cell or cellular subpopulation derived from the large intestine.

Preferably, said percent identity is at least 85%, 90%, 91%, 92%, 93%, 94%, 95%, 96%, 97%, 98% or 99%.

Low stringency includes and encompasses from at least about 1% v/v to at least about 15% v/v formamide and from at least about 1M to at least about 2M salt for hybridisation, and at least about 1M to at least about 2M salt for washing conditions. Alternative stringency conditions may be applied where necessary, such as medium stringency, which includes and encompasses from at least about 16% v/v at least about 30% v/v formamide and from at least about 0.5M to at least about 0.9M salt for hybridisation, and at least about 0.5M to at least about 0.9M salt for washing conditions, or high stringency, which includes and encompasses from at least about 31% v/v to at least about 50% v/v formamide and from at least about 0.01M to at least about 0.15M salt for hybridisation, and at least about 0.01M to at least about 0.15M salt for washing conditions. In general, washing is carried out at Tₘ=69.3+0.41 (G+C) % [19]=−12° C. However, the Tₘ of a duplex DNA decreases by 1° C. with every increase of 1% in the number of mismatched based pairs (Bonner et al (1973) J. Mol. Biol. 81:123).

Preferably, the subject probes are designed to bind to the nucleic acid or protein to which they are directed with a level of specificity which minimises the incidence of non-specific reactivity. However, it would be appreciated that it may not be possible to eliminate all potential cross-reactivity or non-specific reactivity, this being an inherent limitation of any probe based system.

In terms of the probes which are used to detect the subject proteins, they may take any suitable form including antibodies and aptamers.

A library or array of nucleic acid or protein probes provides rich and highly valuable information. Further, two or more arrays or profiles (information obtained from use of an array) of such sequences are useful tools for comparing a test set of results with a reference, such as another sample or stored calibrator. In using an array, individual probes typically are immobilized at separate locations and allowed to react for binding reactions. Primers associated with assembled sets of markers are useful for either preparing libraries of sequences or directly detecting markers from other biological samples.

A library (or array, when referring to physically separated nucleic acids corresponding to at least some sequences in a library) of gene markers exhibits highly desirable properties. These properties are associated with specific conditions, and may be characterized as regulatory profiles. A profile, as termed here refers to a set of members that provides diagnostic information of the tissue from which the markers were originally derived. A profile in many instances comprises a series of spots on an array made from deposited sequences.

A characteristic patient profile is generally prepared by use of an array. An array profile may be compared with one or more other array profiles or other reference profiles. The comparative results can provide rich information pertaining to disease states, developmental state, receptiveness to therapy and other information about the patient.

Another aspect of the present invention provides a diagnostic kit for assaying biological samples comprising an agent for detecting one or more neoplastic marker reagents useful for facilitating the detection by the agent in the first compartment. Further means may also be included, for example, to receive a biological sample. The agent may be any suitable detecting molecule.

The present invention is further described by the following non-limiting examples:

### Example 1

### Methods and Materials

### Affymetrix GeneChip Data

Gene expression profiling data and accompanying clinical data was purchased from GeneLogic Inc (Gaithersburg, Md. USA). For each tissue analysed, oligonucleotide microarray data for 44,928 probesets (Affymetrix HGU133A & HGU133B, combined), experimental and clinical descriptors, and digitally archived microscopy images of histological preparations were received. A quality control analysis was performed to remove arrays not meeting essential quality control measures as defined by the manufacturer.

Transcript expression levels were calculated by both Microarray Suite (MAS) 5.0 (Affymetrix) and the Robust Multichip Average (RMA) normalization techniques (Affymetrix. GeneChip expression data analysis fundamentals. Affymetrix, Santa Clara, Calif. USA, 2001; Hubbell at al. Bioinformatics, 18:1585-1592, 2002; Irizarry at al. Nucleic Acid Research, 31, 2003) MAS normalized data was used for performing standard quality control routines and the final data set was normalized with RMA for all subsequent analyses.

### Univariate Differential Expression

Differentially expressed gene transcripts were identified using a moderated t-test implemented in the limma library downloaded from the Bioconductor repository for R. (G. K. Smyth. Statistical Applications in Genetics and Molecular Biology, 3(1):Article 3, 2004; G K Smyth. Bioinformatics and Computational Biology Solutions using R and Bioconductor. Springer, New York, 2005). Significance estimates (p-values) were corrected to adjust for multiple hypothesis testing using the Bonferonni correction.

### Tissue Specific Expression Patterns

To construct a filter for hypothetically ‘turned off’ gene expression the mean expression level for all 44,928 probesets across the full range of 454 tissues was first estimated. To estimate an expression on/off threshold, the 44,928 mean values were ranked and the expression value equivalent to the 30th percentile across the dataset calculated. This arbitrary threshold was chosen because it was theorized that the majority of transcripts (and presumably more than 30%) in a given specimen should be transcriptionally silenced. Thus this threshold represents a conservative upper bound for what is estimated as non-specific, or background, signal.

### Gene Symbol Annotations

To map Affymetrix probeset names to official gene symbols the annotation metadata available from Bioconductor was used. Hgu133plus2 library version 1.16.0, which was assembled using Entrez Gene data downloaded on 15 Mar. 2007, was used.

### Estimates of Performance Characteristics

Diagnostic utility for each table of markers shown herein was estimated including: sensitivity, specificity, positive predictive value, negative predictive value, likelihood ratio positive, likelihood ratio negative. These estimates were calculated in the same data used to discover the markers and will therefore potentially overestimate the performance characteristics in future tissue samples. To improve the generalisabilty of the estimates a modified jackknife resampling technique was used to calculate a less biased value for each characteristic.

### Results

A range of univariate statistical tests were applied on Affymetrix oligonucleotide microarray data to reveal human genes that could be used to discriminate colorectal neoplastic tissues from non-neoplastic tissues. There were further identified a number of gene transcripts that appear to be useful for differentiating colorectal adenomas from colorectal carcinoma. Also identified were a subset of these transcripts that may have particular diagnostic utility due to the protein products being either secreted or displayed on the cell surface of epithelial cells. Finally, there were identified a further subset of transcripts expressed specifically in neoplastic tissues and at low- or near-background levels in non-neoplastic tissues.

### Genes Differentially Expressed in Neoplastic Tissues

From a total GeneChip set of 44,928 probesets it was determined that over 11,000 probesets were differentially expressed by moderated t-test using the limma package in BioConductor (0. K. Smyth, 2004 supra) employing conservative (Bonferroni) multiple test correction. When this list was further filtered to include only those probesets demonstrating a 2-fold or greater mean expression change between the neoplastic and non-neoplastic tissues, 560 probesets were found to be expressed lower in neoplasias relative to normal tissues.

These 560 probesets were annotated using the most recent metadata and annotation packages available for the chips. The 560 underexpressed probesets were mapped to 434 gene symbols.

| Δ-expression | ProbeSet ID | Gene Symbol Maps |
|---|---|---|
| DOWN | 560 | 434 |

### Hypothetical Markers Specific for Colorectal Neoplasia

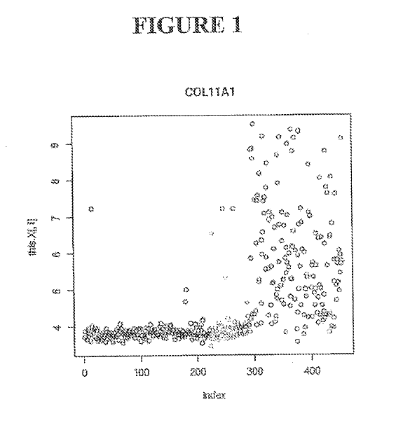

While differential gene expression patterns are useful for diagnostic purposes this project also seeks to identify diagnostic proteins shed into the lumen of the gut by neoplastic colorectal epithelia. To discover candidate proteins, the list of differentially expressed transcripts was filtered with a selection criteria aimed at identifying markers specifically turned off in colorectal neoplasia tissues. To identify ‘off’ genes the filter criteria were designed to find genes with i) neoplastic expression levels below a theoretical on/off threshold and ii) normal signals at least 2-fold higher. The expression profile of an example transcript that is ‘turned-off’ in neoplastic tissues is shown in FIG. 1.

### Example 2

### Probesets Elevated in Non-Neoplasia Relative to Neoplastic Tissues

Differential expression analysis was applied to identify down-regulated probesets in Affymetrix gene chip data measuring RNA concentration in 454 colorectal tissues including 161 adenocarcinoma specimens, 29 adenoma specimens, 42 colitis specimens and 222 non-diseased tissues. Using conservative corrections for multiple hypothesis testing and a 2-fold absolute fold change cut-off it was determined that 560 probesets exhibit a decreased expression level in neoplastic tissues relative to non-neoplastic controls. 560 of these probesets have been mapped to 434 putative gene symbols based on transcript nucleotide sequence.

### Validation/Hypothesis Testing

RNA expression levels of these candidates were measured in independently derived clinical specimens. 526 probesets were hybridised to RNA extracts from 68 clinical specimens comprising 19 adenomas, 19 adenocarcinomas, and 30 non-diseased controls using a custom-designed ‘Adenoma Gene Chip’. Thirty-four (34) probesets were not tested as they were not included on the custom design. It was confirmed that 459 of 526 of the target probesets (or directly related probesets with the same gene locus target) were likewise differentially expressed (P<0.05) in these independently-derived tissues. The results of differential expression analysis of these 459 probesets is shown in Table 1.

The 372 of the 434 unique gene loci to which the 560 probesets are understood to hybridise were further tested. The remaining 62 gene symbols were not represented in the validation data. It was observed that 328 of 372 gene symbols were represented in the validation data by at least one differentially expressed probeset and many symbols included multiple probesets against regions across the putative locus. A complete list of probesets that bind to target loci is shown in Table 2.

### Conclusion

The candidate probesets and symbols shown in Tables 1 and 2. respectively, are differentially expressed lower in neoplastic colorectal tissues compared to non-neoplastic controls.

### Example 3

### Probesets Demonstrating a Non-Neoplasia Specific Profile

During analysis of the discovery data, a novel expression profile was observed between neoplastic and non-neoplastic phenotypes. It was hypothesized that a subset of quantitatively differentially expressed probesets are furthermore qualitatively differentially expressed. Such probesets show no evidence of a gene expression activity in neoplastic tissues, i.e. these probesets appear to be expressed above background levels in non-neoplastic tissues only. This observation and the resulting hypothesis are based on two principles:
1. That the majority of human transcripts that are present on a genome-wide GeneChip (e.g. U133) will not likely be expressed in the colorectal mucosa; and
2. That microarray binding intensity for such ‘off’ probesets (to labeled cRNA) will reflect technical assay background, i.e. non-specific oligonucleotide binding.

To generate a list of non-neoplasia specific probesets the neoplastic intensity of differentially expressed probesets were compared with a hypothetical background signal threshold from across all probesets on the chip. We note that, by design, all probesets in the candidate pool from which the ‘on’ transcripts are chosen are at least two fold over-expressed in the non-diseased tissues relative to diseased tissues. Combined, these criteria yield the subset of differentially expressed transcript species that are specifically expressed in non-neoplastic tissues.

This analysis demonstrated that 42 probesets corresponding to approximately 41 gene loci exhibit a non-neoplasia specific transcription expression profile.

### Validation/Hypothesis Testing

The custom gene chip design precludes testing the non-neoplasia-specific probesets using the same principles as used for discovery. In particular, the custom gene chip (by design) does not contain a large pool of probesets anticipated to hybridise to hypothetically ‘off’/‘non-transcribed’ gene transcripts. This is because the custom gene chip design is heavily biased toward differentially expressed transcripts in colorectal neoplastic tissues.

The usual differential expression testing (limma) was therefore applied to these candidate probesets for specifically expressed in non-neoplastic tissues. Of the 37 (of 42) probesets on the custom gene chip, 33 probesets (or probesets which bind to the same locus) were differentially expressed between the 38 neoplastic tissues (adenoma & cancer) and non-neoplastic controls. The results of these validation experiments is shown in Table 3.

It was further aimed to test all probesets which are known to hybridise to the gene loci to which the probesets claimed herein. Of the 41 putative gene loci targeted by the probesets, 33 were present in the validation data. All thirty-three (33) of these 33 (100%) gene symbols demonstrated at least one hybridising probeset which was differentially expressed in the neoplastic tissues. Results for these experiments, including all probesets that bind to each target locus in a differentially expressed manner are shown in Table 4.

### Example 4

### Materials and Methods for Examples 2 and 3

Gene expression profiling data measured in 454 colorectal tissue specimens including neoplastic, normal and non-neoplastic disease controls was purchased from GeneLogic Inc (Gaithersburg, Md. USA). For each tissue specimen Affymetrix (Santa Clara, Calif. USA) oligonucleotide microarray data totaling 44,928 probesets (HGU133A & HGU133B, combined), experimental and clinical descriptors, and digitally archived microscopy images of histological preparations was received. Prior to applying discovery methods to these data, extensive quality control methods, including statistical exploration, review of clinical records for consistency and histopathology audit of a random sample of arrays was carried out. Microarrays that did not meet acceptable quality criteria were removed from the analysis.

### Hypothesis Testing

Candidate transcription biomarkers were tested using a custom oligonucleotide microarray of 25-mer oligonucleotide probesets designed to hybridise to candidate RNA transcripts identified during discovery. Differential expression hypotheses were tested using RNA extracts derived from independently collected clinical samples comprising 30 normal colorectal tissues, 19 colorectal adenoma tissues, and 19 colorectal adenocarcinoma tissues. Bach RNA extract was confirmed to meet strict quality control criteria.

### Colorectal Tissue Specimens

All tissues used for hypothesis testing were obtained from a tertiary referral hospital tissue bank in metropolitan Adelaide, Australia (Repatriation General Hospital and Flinders Medical Centre). Access to the tissue bank for this research was approved by the Research and Ethics Committee of the Repatriation General Hospital and the Ethics Committee of Flinders Medical Centre. Informed patient consent was received for each tissue studied.

Following surgical resection, specimens were placed in a sterile receptacle and collected from theatre. The time from operative resection to collection from theatre was variable but not more than 30 minutes. Samples, approximately 125 mm3 (5×5×5 mm) in size, were taken from the macroscopically normal tissue as far from pathology as possible, defined both by colonic region as well as by distance either proximal or distal to the pathology. Tissues were placed in cryovials, then immediately immersed in liquid nitrogen and stored at −150° C. until processing.

### RNA Extraction

RNA extractions were performed using Trizol® reagent (Invitrogen, Carlsbad, Calif., USA) as per manufacturer's instructions. Each sample was homogenised in 300 μL of Trizol reagent using a modified Dremel drill and sterilised disposable pestles. Additional 200 μL of Trizol reagent was added to the homogenate and samples were incubated at RT for 10 minutes. 100 μL of chloroform was then added, samples were shaken vortexed for 15 seconds, and incubated at RT for 3 further minutes. The aqueous phase containing target RNA was obtained by centrifugation at 12,000 rpm for 15 min, 40° C. RNA was then precipitated by incubating samples at RT for 10 min with 250μL of isopropanol. Purified RNA precipitate was collected by centrifugation at 12,000 rpm for 10 minutes, 40° C. and supernatants were discarded. Pellets were then washed with 1 mL 75% ethanol, followed by vortexing and centrifugation at 7,500 g for 8 min, 40° C. Finally, pellets were air-dried for 5 min and resuspended in 80 μL of RNase free water. To improve subsequent solubility samples were incubated at 55° C. for 10 min. RNA was quantified by measuring the optical density at A260/280 nm. RNA quality was assessed by electrophoresis on a 1.2% agarose formaldehyde gel.

### Gene Chip Processing

To test hypotheses related to biomarker candidates for colorectal neoplasia RNA extracts were assayed using a custom GeneChip designed by us in collaboration with Affymetrix (Santa Clara, Calif. USA). These custom GeneChips were processed using the standard Affymetrix protocol developed for the HU Gene ST 1.0 array described in (Affy:WTAssay).

### Statistical Software and Data Processing

The R statistics environment R and BioConductor libraries (BioConductor, www.bioconductor.org) (BIOC) was used for most analyses. To map probeset IDs to gene symbol on the Custom GeneChip hgu133plus2 library version 2.2.0 was used which was assembled using Entrez Gene data downloaded on Apr. 18 12:30:55 2008 (BIOC).

### Hypothesis Testing of Differentially Expressed Biomarkers

To assess differential expression between tissue classes the Student's t test for equal means between two samples or the robust variant provided by the limma library (Smyth)(limma) was used. To mitigate the impact of false discovery due to multiple hypothesis testing, a Bonferroni adjustment to P values in the discovery process (MHT:Bonf) was applied. For hypotheses testing the slightly less conservative multiple hypothesis testing correction of Benjamini & Hochberg, which aims to control the false discovery rate of solutions (MHT:BH), was applied.

### Discovery of Tissue-Specific Gene Expression Patterns

Discovery methods using gene expression data often yield numerous candidates, many of which are not suitable for commercial products because they involve subtle gene expression differences that would be difficult to detect in laboratory practice. Pepe et al. note that the ‘ideal’ biomarker is detectable in tumor tissue but not detectable (at all) in non-tumour tissue (Pepe:biomarker:development.) To bias the discovery toward candidates that meet this criteria, an analysis method was developed that aims to enrich the candidates for biomarkers whose qualitative absence or presence measurement is diagnostic for the phenotype of interest. This method attempts to select candidates that show a prototypical ‘turned-on’ or ‘turned-off’ pattern relative to an estimate of the background/noise expression across the chip. It is theorized that such RNA transcripts are more likely to correlate with downstream translated proteins with diagnostic potential or to predict upstream genomic changes (e.g. methylation status) that could be used diagnostically. This focus on qualitative rather than quantitative outcomes may simplify the product development process for such biomarkers.

The method is based on the assumption that the pool of extracted RNA species in any given tissue (e.g. colorectal mucosae) will specifically bind to a relatively small subset of the full set of probesets on a GeneChip designed to measure the whole genome. On this assumption, it is estimated that most probesets on a full human gene chip will not exhibit specific, high-intensity signals.

This observation is utilised to approximate the background or ‘non-specific binding’ across the chip by choosing a theoretical level equal to the value of e.g. lowest 25% quantile of the ranked mean values. This quantile can be arbitrarily set to some level below which there is made a reasonable assumption that the signals do not represent above-background RNA binding. Finally, this background estimate is used as a threshold to estimate the ‘OFF’ probesets in an experiment for, say, the non-neoplastic tissue specimens.

Conversely, it is further hypothesized that probesets which are 1) expressed above this theoretical threshold level and 2) at differentially higher levels in the tumour specimens may be a tumour specific candidate biomarker. It is noted that in this case the concept of ‘fold-change’ thresholds can also be conveniently applied to further emphasize the concept of absolute expression increases in a putatively ‘ON’ probeset.

Given the assumption of low background binding for a sizeable fraction of the measured probesets, this method was only used in the large GeneLogic data and discovery. To construct a filter for hypothetically ‘turned on’ biomarker in the GeneLogic discovery data, the mean expression level for all 44,928 probesets across the full range of 454 tissues was estimated. The 44,928 mean values were then ranked and the expression value equivalent to the 25th percentile across the dataset calculated. This arbitrary threshold was chosen because the majority of transcripts (and presumably more than 25%) in a given specimen should exhibit low concentration which effectively transcriptional silence. Thus this threshold represents a conservative upper bound for what is estimated is non-specific, or background, expression.

### Example 5

### DNA Methylation Data

Assays were developed for detection of methylation in the promoter regions the eight down-regulated genes in Table 5. Methods for bisulphite treatment of DNA and assays for determination of DNA methylation levels, including MSP and COBRA, are described in Clark et al., (2006).

Five MSP assays used the primer pairs shown in TABLE 7. A control PCR for unbiased amplification of the CAGE gene was used to determine the quantity of input DNA to provide a reference for quantification of the level of methylation of each gene. For PCRs, 25 μL reactions in Biorad iQ SyBr Green Super Mix contained 5 ng of bisulphite-treated DNAs (1 ng for cell line assays and 6 ng for clinical specimens) and 200 nM of forward and reverse primers. PCR cycling conditions were:
95.0° C. for 2 min
Followed by 50 cycles of
95.0° C./15 sec
Temp® C/30 sec
72.0° C./30 sec

Where “Temp” is the re-annealing temperature optimised for each gene as shown in Table yy.

For the DF gene, 3 preliminary cycles were done using a 95.0° C. melting temperature, followed by 50 cycles with a lower, 84.0° C. melting temperature (to reduce nonspecific amplification).

A standard curve was generated using DNA methylated with M.SssI methylase (100% methylated) and DNA that had been in vitro amplified using Phi29 DNA polymerase (0% methylation).

COBRA assays were developed for three genes as shown in TABLE 8. PCRs were setup as above with cycling conditions:
95.0° C. for 2 min
Followed by 50 cycles of
95.0° C./15 sec
Temp® C/30 sec
72.0° C./30 sec

After PCR, 10 μL of PCR product was digested with the appropriate enzyme (TABLE 8), digestion products analysed by gel electrophoresis and methylation levels determined semiquantitatively.

The methylation state of the eight genes was determined in four colorectal cancer cell lines, Caco2, HCT116, HT29 and SW480 as well as normal blood DNA and the normal lung fibroblast cell line, MRC5. The level of methylation in summarised in Table 5. The promoter regions of all eight genes show strong methylation in 2 or 3 of the four colorectal cancer cell lines tested. All showed a lack or low level of methylation in DNA from normal blood DNA and the fibroblast cell line MRC5, except for methylation of DF in MRC5.

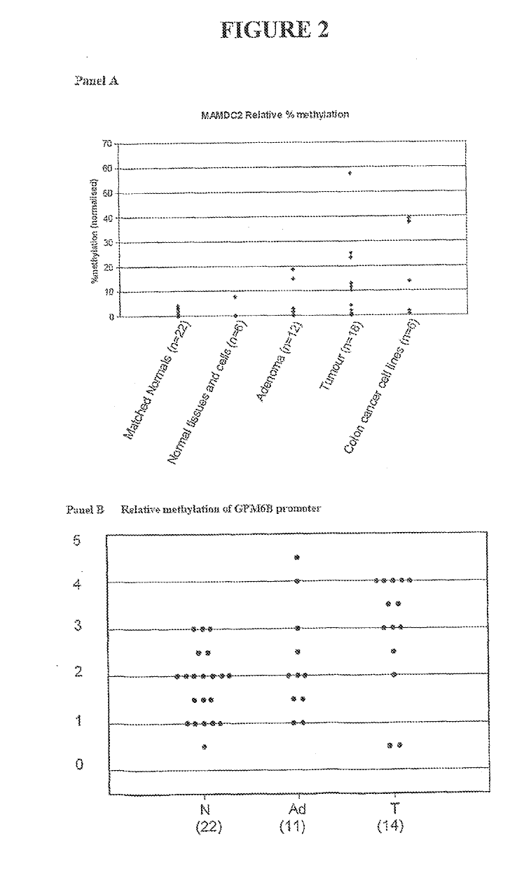

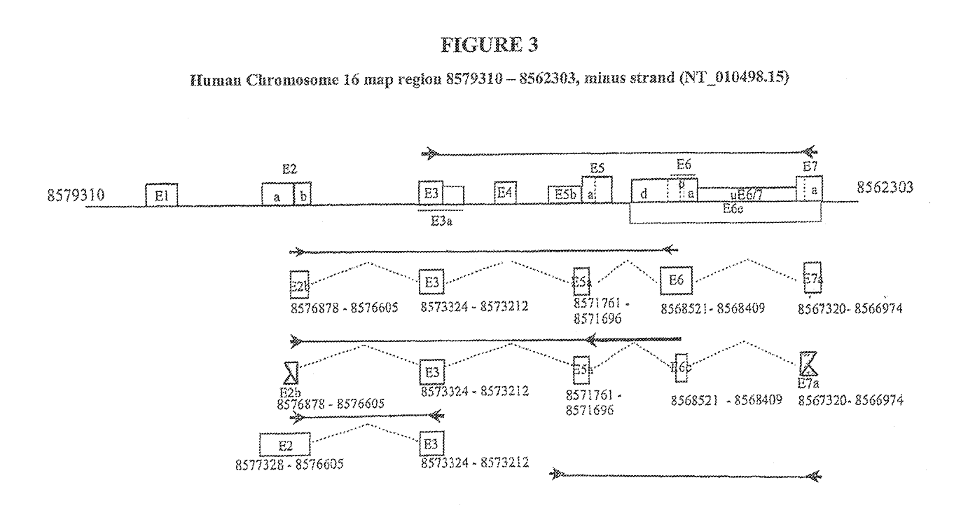

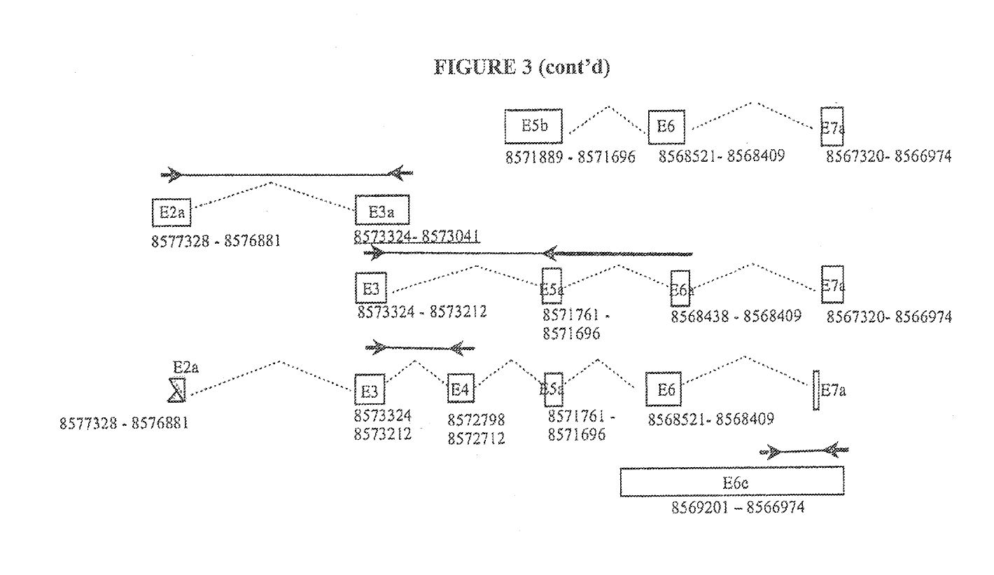

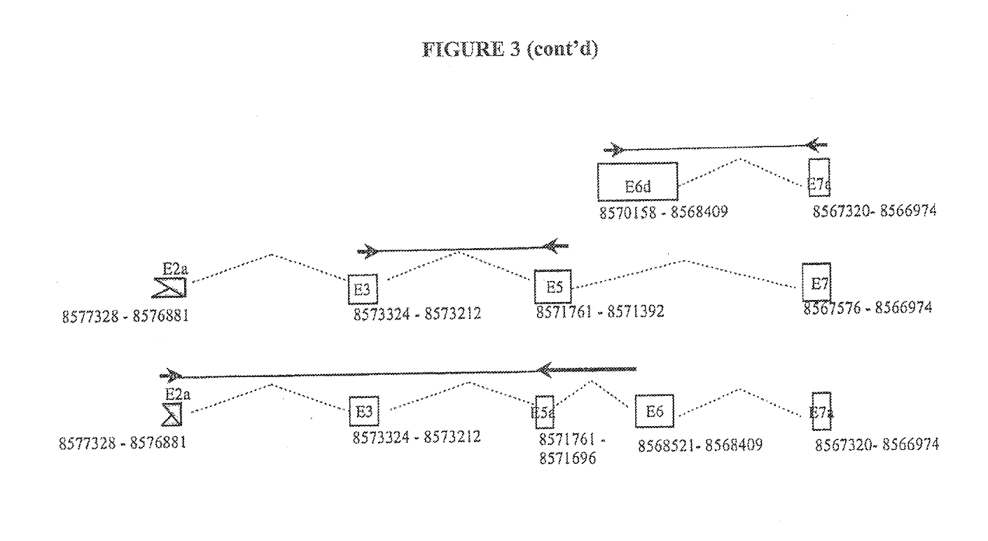

For two of these genes, MAMDC2 and GPM6B analysis has been extended to a set of 12 adenoma, 18 cancer and 22 matched normal tissue samples (FIGS. 2, A and B).

For MAMDC2 quantitative analysis demonstrated that 2 of 12 adenomas and 6 of 18 cancer samples showed elevated methylation compared with the highest level observed in normal tissue samples. Methylation levels of the GPM6B gene were determined by semiquantitative COBRA assays, scored on a scale of 0 to 5 based on visual inspection of restriction digestions. A clear trend toward increasing promoter methylation in progression from normal to adenoma to cancer was evident (FIG. 2, panel B).

These data demonstrate for a number of examples of the down-regulated genes that such downregulation in colorectal cancer cell lines and primary neoplasia tissue may be associated with DNA methylation and that assays of DNA methylation can be used to discriminate cancer and normal tissue.

### Example 6

### Determine Gene Identity of a Nucleic Add Sequence of Interest which is Define by an Affymetrix Probeset

BLAST the Sequence of Interest Using Online Available Basic Local Alignment Search Tools [BLAST]. e.g. NCBI/BLAST
(http://blast.nbi.nlm.nih.gov/Blast.cgi)
(a) Select “Human” in BLAST ASSEMBLED GENOMES on the web page http://blast.ncbi.nlm.nih.gov/Blast.cgi
(b) Leave the default settings, i.e.:
   Database: Genome (all assemblies)
   Program: megaBLAST: compare highly related nucleotide sequences
   Optional parameters: Expect: 0.01, Filter: default, Descriptions: 100, Alignments: 100
(c) Copy/Paste Sequence into the “BLAST” window
(d) Click “Begin Search”
(e) Click “View Report”

### Assessment of the Open BLAST Search Results

Multiple significant sequence alignments may be identified when “blasting” the sequence.

### Identify Gene Nomenclature of the Identified Sequence Match

(a) Click the link to one of the identified hits
(b) The new page will schematically depict the position of the hit on one chromosome. It will be apparent which gene is hit.
(c) Retrieve the “hit” sequence clicking on the link
(d) Do a search for the gene in the provided “search” window. This provides the gene nucleotide coordinates for the gene.

### Determine Promiscuity of Sequence

(a) Open the NCBI/BLAST tool, (http://blast.ncbi.nlm.nih.gov/Blast.cgi)
(b) Click on “nucleotide Blast” under “basic BLAST”
(c) Copy/paste the sequence of interest into the “Query Sequence” window
(d) Click “Blast”.
   Assessment of the nBLAST Search Results of the Sequence
(a) The nBLAST exercise with the Sequence may result in multiple Blast hits of which some accession entry numbers are listed in “Description”.
(b) These hits should be reviewed.

### Determine Location of the Sequence in the Gene

The Ensembl database is an online database, which produces and maintains automatic annotation selected eukaryotic genomes (www.ensembl.org/index.html)

### Identify Location of the Sequence in the Gene

(a) Set “Search” to Homo Sapiens, Type “the gene name” in the provided Search Field Ensemble.org/index.html)
(b) Click “Go”
(c) Click the “vega protein_coding Gene: OTTHUMG000000144184” link to get an annotation report
(d) Click on “Gene DAS Report” to retrieve information regarding Alternative splice site database: Type “the gene name” in search field
   Click on “the gene entry”
   Scroll down to “evidences”
   Review alternative splice sites
   Click “Confirmed intron/exons” to get a list of coordinates for the exons & introns.
      Alternative Splicing and/or Transcription
      The AceView Database provides curated and non-redundant sequence representation of all public mRNA sequences. The database is available through NCBI: http://www.ncb.nlm.nih.gov/IEB/Research/Acembly/
      Further Investigation of the Gene mRNA Transcripts
(a) Type “the gene name” into the provided “search” field
(b) Click “Go”
(c) The following information is available from the resulting entry in AceView:
   The number of cDNA clones from which the gene is constructed (ie originated-from experimental work involving isolation of mRNA)
   The mRNAs predicted to be produced by the gene
   The existence of non-overlapping alternative exons and validated alternative polyadenylation sites
   The existence of truncations
   The possibility of regulated alternate expression
   Introns recorded as participating in alternatively splicing of the gene
(d) Classic splice site motives

### Application of Method to LOC643911/hCG_1815491

### Materials and Methods

### Extraction of RNA

RNA extractions were performed using Trizol® reagent (Invitrogen, Carlsbad, Calif., USA) as per manufacturer's instructions. Each sample was homogenised in 300 μL of Trizol reagent using a modified dremel drill and sterilised disposable pestles. Additional 200 μL of Trizol reagent was added to the homogenate and samples were incubated at RT for 10 minutes. 100 μL of chloroform was then added, samples were shaken vortexed for 15 seconds, and incubated at RT for 3 further minutes. The aqueous phase containing target RNA was obtained by centrifugation at 12,000 rpm for 15 min, 40° C. RNA was then precipitated by incubating samples at RT for 10 min with 250 μL of isopropanol. Purified RNA precipitate was collected by centrifugation at 12,000 rpm for 10 minutes, 40° C. and supernatants were discarded. Pellets were then washed with 1 mL 75% ethanol, followed by vortexing and centrifugation at 7,500 g for 8 min, 40° C. Finally, pellets were air-dried for 5 min and resuspended in 80 μL of RNase free water. To improve subsequent solubility samples were incubated at 55, C for 10 min. RNA was quantified by measuring the optical density at A260/280 nm. RNA quality was assessed by electrophoresis on a 1.2% agarose formaldehyde gel.

### Gene Chip Processing

RNA samples to analyze on Human Exon 1.0 ST GeneChips were processed using the Affymetrix WT target labeling and control kit (part#900652) following the protocol described in (Affymetrix 2007 P/N 701880 Rev.4). Briefly: First cycle cDNA was synthesized from 100 ng ribosomal reduced RNA using random hexamer primers tagged with T7 promoter sequence and SuperScript II (Invitrogen, Carlsbad Calif.), this was followed by DNA Polymerase I synthesis of the second strand cDNA. Anti-sense cRNA was then synthesized using T7 polymerase. Second cycle sense cDNA was then synthesised using SuperScript II, dNTP+dUTP, and random hexamers to produce sense strand cDNA incorporating uracil. This single stranded uracil containing cDNA was then fragmented using a combination of uracil DNA glycosylase (UDG) and apurinic/apyrimidinic endonuclease1 (APE 1). Finally the DNA was biotin labelled using terminal deoxynucleotidyl transferase (TdT) and the Affymetrix proprietary DNA Labeling reagent. Hybridization to the arrays was carried out at 45° C. for 16-18 hours.

Washing and staining of the hybridized GeneChips was carried out using the Affymetrix Fluidics Station 450 and scanned with the Affymetrix Scanner 3000 following recommended protocols.

### SYBR Green Based Quantitative Real Time-PCR

Quantitative real time polymerase chain reaction was performed on RNA isolated from clinical samples for the amplification and detection of the various hCG_1815491 transcripts.

Firstly cDNA was synthesized from 2 ug of total RNA using the Applied Biosystems High Capacity Reverse transcription Kit (P/N 4368814). After synthesis the reaction was diluted 1:2 with water to obtain a final volume of 40 ul and 1 ul of this diluted cDNA used in subsequent PCR reactions.

PCR was performed in a 25 ul volume using 12.5 ul Promega 2×PCR master mix (P/N M7502), 1.5 ul 5 uM forward primer, 1.5 ul 5 uM reverse primer, 7.875 ul water, 0.625 ul of a 1:3000 dilution of 10,000× stock of SYBR green 1 pure dye (Invitrogen P/N S7567), and 1 ul of cDNA.

Cycling conditions for amplification were 95° for 2 minutes×1 cycle, 95° for 15 seconds and 60° for 1 minute×40 cycles. The amplification reactions were performed in a Corbett Research Rotor-Gene RG3000 or a Roche LightCycler480 real-time PCR machine. When the Roche LightCycler480 real-time PCR machine was used for amplification the reaction volume was reduced to 10 ul and performed in a 384 well plate but the relative ratios between all the components remained the same. Final results were calculated using the ΔΔCt method with the expression levels of the various hCG_1815491 transcripts being calculated relative to the expression level of the endogenous house keeping gene HPRT.

### End-Point PCR

End point PCR was performed on RNA isolated from clinical samples for the various hCG_1815491 transcripts. Conditions were identical to those described for the SYBR green assay above but with the SYBR green dye being replaced with water. The amplification reactions were performed in a MJ Research PTC-200 thermal cycler. 2.5 μl of the amplified products were analysed on 2% agarose E-gel (Invitrogen) along with a 100-base pair DNA Ladder Marker.

### Results

The nucleotide structure and expression levels of transcripts related to hCG_1815491 was analysed based on the identification of diagnostic utility of Affymetrix probesets 238021_s_at and 238022_at from the gene chip analysis.

The gene hCG_1815491 is currently represented in NCBI as a single RefSeq sequence, XM_93911. The RefSeq sequence of hCG_1815491 is based on 89 GenBank accessions from 83 cDNA clones. Prior to March 2006, these clones were predicted to represent two overlapping genes, LOC388279 and LOC650242 (the latter also known as LOC643911). In March 2006, the human genome database was filtered against clone rearrangements, co-aligned with the genome and clustered in a minimal non-redundant way. As a result, LOC388272 and LOC650242 were merged into one gene named hCG_1815491 (earlier references to hCG_1815491 are: LOC388279, LOC643911, LOC650242, XM_944116, AF275804, XM373688).

It has been determined that the Ref Sequence, which is defined by the genomic coordinates 8579310 to 8562303 on human chromosome 16 as defined by the NCBI contig reference NT_010498.15|Hs16_10655, NCBI 36 Mar. 2006 genome encompasses hCG_1815491. The 10 predicted RNA variants derived from this gene have been aligned with the genomic nucleotide sequence residing in the map region 8579310 to 8562303. This alignment analysis revealed the existence of at least 6 exons of which several are alternatively spliced. The identified exons are in contrast to the just 4 exons specified in the NCBI hCG_1815491 RefSeq XM_93911. Two additional putative exons were also identified in the Ref Sequence by examination of included probesets on Affymetrix Genechip HuGene Exon 1.0 that target nucleotide sequences embedded in the Ref Sequence. The identified and expanded exon-intron structure of hCG_1815491 have been used to design specific oligonucleotide primers, which allowed measurement of the expression of RNA variants generated from the Ref Sequence by using PCR-based methodology (FIG. 4)

### Tables

The probeset designations include both HG-133plus2 probeset IDs and Human Gene 1.0ST array probe ids. The latter can be conveniently mapped to Transcript Cluster ID using the Human Gene 1.0ST probe tab file provided by Affymetrix (http://www.affymetrix.com/Auth/analysis/downloads/na22/wtgene/HuGene-1_0-st-v1.probe.tab.zip). Using publicly available software such as NetAffx (provided by Affymetrix), the Transcript Cluster ID may be further mapped to gene symbol, chromosomal location, etc.

### Table 1

Probesets demonstrated to be expressed higher in non-neoplastic tissues relative to neoplastic controls. TargetPS: Affymetrix HG-U133plus2 probeset id; Symbol: putative gene symbol corresponding to target probeset id—multiple symbol names indicate the possibility of probeset hybridisation to multiple gene targets; Signif. FDR: Adjusted p-value for mean difference testing between RNA extracted from neoplasia and non-neoplastic tissues. Adjustment is made using Benjamini & Hochberg correction for multiple hypothesis testing (Benjamini and Hochberg, 1995); D.value50: Diagnostic effectiveness parameter estimate corresponding to the area of a receiver operator characteristic ROC. This parameter provides a convenient estimate of diagnostic utility and is described in (Saunders, 2006); FC: fold change between mean expression level of non-neoplasia vs. neoplasia; Sens-Spec: Estimate of diagnostic performance corresponding to the ROC curve point demonstrating equal sensitivity and specificity; CI (95): 95% confidence interval of sensitivity and specificity estimates.

### Table 2

Evidence of multiple probesets which correspond to gene symbols claimed herein exhibiting RNA concentration differences between non-neoplastic tissues and neoplastic controls. Symbol: gene symbol; ValidPS_DOWN: Affymetrix probeset IDs demonstrating statistically significant overexpression in non-neoplastic RNA extracts relative to neoplastic controls. Signif. FDR. Adjusted p-value for mean difference testing between RNA extracted from neoplasia and non-neoplastic tissues. Adjustment is made using Benjamini & Hochberg correction for multiple hypothesis testing (Benjamini and Hochberg, 1995); D.value50: Diagnostic effectiveness parameter estimate corresponding to the area of a receiver operator characteristic ROC. This parameter provides a convenient estimate of diagnostic utility and is described in (Saunders, 2006); FC: fold change between mean expression level of non-neoplasia vs. neoplasia; Sens-Spec: Estimate of diagnostic performance corresponding to the ROC curve point demonstrating equal sensitivity and specificity; CI (95): 95% confidence interval of sensitivity and specificity estimates.

### Table 3

Probesets which demonstrate a qualitatively (in addition to quantitative) elevated profile in non-neoplastic tissues relative to neoplastic controls. TargetPS: Affymetrix HG-U133plus2 probeset id; Symbol: putative gene symbol corresponding to target probeset id—multiple symbol names indicate the possibility of probeset hybridisation to multiple gene targets; Signif. FDR: Adjusted p-value for mean difference testing between RNA extracted from neoplasia and non-neoplastic tissues. Adjustment is made using Benjamini & Hochberg correction for multiple hypothesis testing (Benjamini and Hochberg, 1995); D.value50: Diagnostic effectiveness parameter estimate corresponding to the area of a receiver operator characteristic ROC. This parameter provides a convenient estimate of diagnostic utility and is described in (Saunders, 2006); FC: fold change between mean expression level of non-neoplasia vs. neoplasia; Sens-Spec: Estimate of diagnostic performance corresponding to the ROC curve point demonstrating equal sensitivity and specificity; CI (95): 95% confidence interval of sensitivity and specificity estimates.

### Table 4

Evidence of multiple probesets which correspond to gene symbols claimed herein exhibiting qualitative changes in RNA concentration in non-neoplastic tissues compared to neoplastic tissues. Symbol: gene symbol; ValidPS_DOWN: Affymetrix probeset IDs demonstrating statistically significant overexpression in neoplastic RNA extracts relative to non-neoplastic controls. Signif. FDR Adjusted p-value for mean difference testing between RNA extracted from neoplasia and non-neoplastic tissues. Adjustment is made using Benjamini & Hochberg correction for multiple hypothesis testing (Benjamini and Hochberg, 1995); D.value50: Diagnostic effectiveness parameter estimate corresponding to the area of a receiver operator characteristic ROC. This parameter provides a convenient estimate of diagnostic utility and is described in (Saunders, 2006); FC: fold change between mean expression level of non-neoplasia vs. neoplasia; Sens-Spec: Estimate of diagnostic performance corresponding to the ROC curve point demonstrating equal sensitivity and specificity; CI (95): 95% confidence interval of sensitivity and specificity estimates.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations of any two or more of said steps or features.

TABLE 1

| | | | | | Sens- | |
|---|---|---|---|---|---|---|
| TargetPS | Symbol | Signif. FDR | D.val5 | FC | Spec | CI (95) |
| 230788_at | SPTLC3:GCNT2 | 2.16E−27 | 3.9512 | 13.36 | 97.6 | 94.2-99.2 |
| 207502_at | GUCA2B | 7.04E−25 | 3.6279 | 51.78 | 96.5 | 92.3-98.6 |
| 207003_at | GUCA1B | 7.32E−25 | 3.565 | 10.7 | 96.3 | 92-98.5 |
| 206208_at | CA4 | 1.73E−24 | 3.6263 | 10.41 | 96.5 | 92.4-98.6 |
| 206209_s_at | CA4 | 1.73E−24 | 3.6265 | 10.41 | 96.5 | 92.3-98.6 |
| 203908_at | LOC727995:SLC4A4:LOC730895 | 2.23E−23 | 3.6298 | 3.58 | 96.5 | 92.4-98.6 |
| 206784_at | AQP8 | 2.98E−22 | 3.3284 | 10.82 | 95.2 | 90.3-97.9 |
| 205950_s_at | CA1 | 3.15E−22 | 3.368 | 38.27 | 95.4 | 90.6-98 |
| 209735_at | ABCG2 | 1.09E−21 | 3.2584 | 31.53 | 94.8 | 89.7-97.7 |
| 230830_at | OSTbeta | 6.23E−21 | 3.1861 | 7.15 | 94.4 | 89.2-97.5 |
| 223754_at | MGC13057 | 6.94E−21 | 3.2036 | 3.18 | 94.5 | 89.3-97.5 |
| 228195_at | MGC13057 | 6.94E−21 | 3.2023 | 3.18 | 94.5 | 89.4-97.5 |
| 228706_s_at | CLDN23 | 4.27E−20 | 3.0757 | 3.47 | 93.8 | 88.2-97.1 |
| 228707_at | CLDN23 | 4.27E−20 | 3.072 | 3.47 | 93.8 | 88.3-97.1 |
| 223551_at | PKIB | 5.94E−20 | 3.1219 | 3.28 | 94.1 | 88.6-97.3 |
| 231120_x_at | PKIB | 5.94E−20 | 3.1226 | 3.28 | 94.1 | 88.6-97.3 |
| 226492_at | SEMA6D | 6.07E−20 | 3.0898 | 4.27 | 93.9 | 88.4-97.2 |
| 220026_at | CLCA4 | 1.25E−19 | 3.1909 | 16.3 | 94.5 | 89.2-97.5 |
| 224836_at | TP53INP2 | 1.45E−19 | 3.0248 | 3.78 | 93.5 | 87.9-96.9 |
| 220834_at | MS4A12 | 2.13E−19 | 3.29 | 7.15 | 95 | 90-97.8 |
| 224412_s_at | TRPM6 | 2.25E−19 | 3.01 | 7.5 | 93.4 | 87.7-96.8 |
| 220037_s_at | XLKD1 | 3.60E−19 | 2.9412 | 7.44 | 92.9 | 87.1-96.6 |
| 219059_s_at | XLKD1 | 3.60E−19 | 2.942 | 7.44 | 92.9 | 87-96.6 |
| 209612_s_at | ADH1B:ADH1A | 4.70E−19 | 3.0183 | 4.67 | 93.4 | 87.7-96.9 |
| 209613_s_at | ADH1B:ADH1A | 4.70E−19 | 3.0147 | 4.67 | 93.4 | 87.7-96.9 |
| 200845_s_at | LOC389249:PRDX6 | 5.56E−19 | 2.8412 | 2.76 | 92.2 | 86.1-96.1 |
| 209301_at | CA2 | 5.87E−19 | 2.9662 | 9.22 | 93.1 | 87.3-96.7 |
| 208399_s_at | EDN3 | 6.42E−19 | 2.9342 | 11.21 | 92.9 | 87-96.5 |
| 228961_at | MIER3 | 9.14E−19 | 2.9183 | 3.91 | 92.8 | 86.9-96.5 |
| 231975_s_at | MIER3 | 9.14E−19 | 2.9172 | 3.91 | 92.8 | 86.8-96.4 |
| 204719_at | ABCA8 | 1.26E−18 | 2.9172 | 6.82 | 92.8 | 86.8-96.5 |
| 207761_s_at | METTL7A | 1.35E−18 | 3.1247 | 1.93 | 94.1 | 88.6-97.3 |
| 207977_s_at | DPT | 1.53E−18 | 2.8967 | 9.07 | 92.6 | 86.6-96.4 |
| 213068_at | DPT | 1.53E−18 | 2.8973 | 9.07 | 92.6 | 86.7-96.4 |
| 213071_at | DPT | 1.53E−18 | 2.8962 | 9.07 | 92.6 | 86.7-96.4 |
| 218756_s_at | MRM1:MGC4172 | 1.74E−18 | 2.9041 | 3.74 | 92.7 | 86.6-96.4 |
| 212288_at | FNBP1:C1orf85:CCT3 | 1.76E−18 | 3.0475 | 2.04 | 93.6 | 88-97 |
| 204036_at | EDG2 | 2.44E−18 | 2.8853 | 3.19 | 92.5 | 86.4-96.3 |
| 202037_s_at | SFRP1 | 2.77E−18 | 2.8647 | 15.53 | 92.4 | 86.3-96.2 |
| 206143_at | SLC26A3 | 4.18E−18 | 2.9202 | 21.4 | 92.8 | 86.9-96.5 |
| 215657_at | SLC26A3 | 4.18E−18 | 2.9226 | 21.4 | 92.8 | 86.9-96.5 |
| 231773_at | ANGPTL1 | 1.42E−17 | 2.7938 | 4.64 | 91.9 | 85.6-95.9 |
| 205480_s_at | UGP2 | 1.64E−17 | 2.7911 | 2.41 | 91.9 | 85.6-95.9 |
| 204955_at | SRPX | 1.79E−17 | 2.7753 | 3.91 | 91.7 | 85.5-95.8 |
| 222722_at | OGN | 2.25E−17 | 2.7048 | 13.67 | 91.2 | 84.8-95.4 |
| 202555_s_at | MYLK | 2.33E−17 | 2.773 | 3.99 | 91.7 | 85.4-95.8 |
| 224823_at | MYLK | 2.33E−17 | 2.7727 | 3.99 | 91.7 | 85.4-95.8 |
| 225575_at | LIFR | 2.45E−17 | 2.7823 | 6.36 | 91.8 | 85.5-95.8 |
| 214142_at | LOC653808:ZG16 | 2.51E−17 | 2.8119 | 8.14 | 92 | 85.8-95.9 |
| 206710_s_at | EPB41L3 | 2.62E−17 | 2.8298 | 2.51 | 92.1 | 85.9-96.1 |
| 205464_at | SCNN1B | 3.20E−17 | 2.7209 | 16.15 | 91.3 | 84.9-95.5 |
| 220812_s_at | HHLA2 | 3.47E−17 | 2.6717 | 10.02 | 90.9 | 84.4-95.3 |
| 203913_s_at | HPGD | 3.65E−17 | 2.8441 | 3.21 | 92.2 | 86.1-96.1 |
| 203914_x_at | HPGD | 3.65E−17 | 2.8456 | 3.21 | 92.3 | 86.1-96.1 |
| 211548_s_at | HPGD | 3.65E−17 | 2.8446 | 3.21 | 92.3 | 86.1-96.1 |
| 211549_s_at | HPGD | 3.65E−17 | 2.8466 | 3.21 | 92.3 | 86.1-96.1 |
| 206198_s_at | CEACAM7 | 3.96E−17 | 2.7882 | 10.4 | 91.8 | 85.5-95.9 |
| 206199_at | CEACAM7 | 3.96E−17 | 2.7855 | 10.4 | 91.8 | 85.6-95.8 |
| 211848_s_at | CEACAM7 | 3.96E−17 | 2.7882 | 10.4 | 91.8 | 85.6-95.8 |
| 226430_at | SMAD5:RELL1 | 4.10E−17 | 2.6968 | 2.5 | 91.1 | 84.6-95.4 |
| 202992_at | C7 | 4.40E−17 | 2.7163 | 6.67 | 91.3 | 84.9-95.5 |
| 205112_at | PLCE1 | 6.91E−17 | 2.7542 | 2.43 | 91.6 | 85.2-95.7 |
| 229839_at | SCARA5 | 1.25E−16 | 2.6897 | 2.86 | 91.1 | 84.6-95.3 |
| 235849_at | SCARA5 | 1.25E−16 | 2.6918 | 2.86 | 91.1 | 84.5-95.3 |
| 209763_at | CHRDL1 | 1.49E−16 | 2.6093 | 13.88 | 90.4 | 83.7-94.9 |
| 205259_at | NR3C2 | 2.08E−16 | 2.5812 | 3.19 | 90.2 | 83.4-94.7 |
| 202242_at | TSPAN7 | 2.13E−16 | 2.6519 | 3.68 | 90.8 | 84.1-95.1 |
| 203000_at | STMN2 | 2.25E−16 | 2.6115 | 6.13 | 90.4 | 83.7-94.9 |
| 203001_s_at | STMN2 | 2.25E−16 | 2.6141 | 6.13 | 90.4 | 83.7-94.9 |
| 209074_s_at | FAM107A | 2.52E−16 | 2.6171 | 2.92 | 90.5 | 83.8-94.9 |
| 202920_at | ANK2 | 2.59E−16 | 2.6499 | 6.76 | 90.7 | 84.1-95.1 |
| 213317_at | CLIC5 | 3.68E−16 | 2.696 | 2.37 | 91.1 | 84.6-95.4 |
| 204697_s_at | CHGA | 5.54E−16 | 2.6087 | 7.21 | 90.4 | 83.7-94.9 |
| 212814_at | KIAA0828 | 5.58E−16 | 2.5495 | 4.09 | 89.9 | 83-94.5 |
| 225275_at | EDIL3 | 6.21E−16 | 2.6252 | 2.96 | 90.5 | 83.9-95 |
| 208370_s_at | DSCR1 | 6.21E−16 | 2.6146 | 2.14 | 90.4 | 83.7-94.9 |
| 209147_s_at | PPAP2A | 6.43E−16 | 2.5961 | 2.4 | 90.3 | 83.6-94.8 |
| 210946_at | PPAP2A | 6.43E−16 | 2.5967 | 2.4 | 90.3 | 83.5-94.8 |
| 202731_at | PDCD4 | 8.67E−16 | 2.4558 | 2.6 | 89 | 82-94 |
| 219799_s_at | DHRS9:GORASP2 | 1.09E−15 | 2.7368 | 1.51 | 91.4 | 85.1-95.6 |
| 223952_x_at | DHRS9:GORASP2 | 1.09E−15 | 2.7334 | 1.51 | 91.4 | 85-95.6 |
| 224009_x_at | DHRS9:GORASP2 | 1.09E−15 | 2.7359 | 1.51 | 91.4 | 85.1-95.6 |
| 236313_at | CDKN2B | 1.34E−15 | 2.5442 | 10.09 | 89.8 | 83-94.5 |
| 231925_at | P2RY1 | 1.41E−15 | 2.5354 | 3.6 | 89.8 | 82.9-94.4 |
| 238143_at | LOC646627 | 1.58E−15 | 1.9142 | 5.02 | 83.1 | 74.8-89.4 |
| 224480_s_at | LPAAT-THETA | 2.06E−15 | 2.3867 | 2.63 | 88.4 | 81.1-93.5 |
| 212230_at | PPAP2B | 2.44E−15 | 2.5821 | 1.81 | 90.2 | 83.4-94.7 |
| 207080_s_at | PYY | 3.50E−15 | 2.4811 | 11.71 | 89.3 | 82.3-94.1 |
| 205200_at | CLEC3B | 4.65E−15 | 2.4426 | 4.46 | 88.9 | 81.8-93.8 |
| 228133_s_at | NDE1 | 4.84E−15 | 2.514 | 1.99 | 89.6 | 82.7-94.3 |
| 214038_at | CCL8 | 5.75E−15 | 2.4504 | 7.51 | 89 | 81.9-93.9 |
| 219014_at | PLAC8 | 5.76E−15 | 2.4248 | 3.87 | 88.7 | 81.6-93.7 |
| 219796_s_at | MUCDHL | 5.81E−15 | 2.4346 | 3.06 | 88.8 | 81.6-93.8 |
| 220075_s_at | MUCDHL | 5.81E−15 | 2.4354 | 3.06 | 88.8 | 81.7-93.8 |
| 215299_x_at | SULT1A1:SULT1A2 | 7.17E−15 | 2.4231 | 3.37 | 88.7 | 81.5-93.7 |
| 233565_s_at | SDCBP2 | 9.28E−15 | 2.4871 | 2.37 | 89.3 | 82.3-94.1 |
| 228885_at | RPL24:LOC731365 | 1.23E−14 | 2.5555 | 1.63 | 89.9 | 83.1-94.6 |
| 209687_at | CXCL12 | 1.54E−14 | 2.4322 | 3.79 | 88.8 | 81.7-93.8 |
| 218546_at | C1orf115 | 1.95E−14 | 2.4106 | 2.95 | 88.6 | 81.4-93.6 |
| 205097_at | SLC26A2 | 2.08E−14 | 2.4219 | 7.83 | 88.7 | 81.5-93.7 |
| 224959_at | SLC26A2 | 2.08E−14 | 2.4229 | 7.83 | 88.7 | 81.6-93.7 |
| 224963_at | SLC26A2 | 2.08E−14 | 2.4205 | 7.83 | 88.7 | 81.5-93.7 |
| 204069_at | MEIS1 | 2.49E−14 | 2.3759 | 5.44 | 88.3 | 81-93.4 |
| 223121_s_at | SFRP2 | 3.05E−14 | 2.3937 | 7.39 | 88.4 | 81.2-93.5 |
| 223122_s_at | SFRP2 | 3.05E−14 | 2.3967 | 7.39 | 88.5 | 81.2-93.5 |
| 209191_at | TUBB3:MC1R:TUBB6 | 3.55E−14 | 2.3684 | 5.9 | 88.2 | 80.9-93.3 |
| 201348_at | GPX3 | 5.68E−14 | 2.36 | 2.57 | 88.1 | 80.8-93.2 |
| 214091_s_at | GPX3 | 5.68E−14 | 2.3613 | 2.57 | 88.1 | 80.7-93.3 |
| 228766_at | CD36 | 6.27E−14 | 2.4671 | 1.65 | 89.1 | 82.1-94 |
| 221896_s_at | HIGD1A | 6.29E−14 | 2.3985 | 2.09 | 88.5 | 81.3-93.5 |
| 201865_x_at | NR3C1 | 7.65E−14 | 2.3539 | 2.51 | 88 | 80.6-93.2 |
| 211671_s_at | NR3C1 | 7.65E−14 | 2.3517 | 2.51 | 88 | 80.7-93.2 |
| 206149_at | LOC63928 | 8.09E−14 | 2.3513 | 3.2 | 88 | 80.7-93.2 |
| 228846_at | MXD1 | 8.35E−14 | 2.4074 | 2.11 | 88.6 | 81.4-93.6 |
| 225602_at | C9orf19 | 1.74E−13 | 2.3619 | 1.95 | 88.1 | 80.9-93.3 |
| 225604_s_at | C9orf19 | 1.74E−13 | 2.3646 | 1.95 | 88.1 | 80.8-93.3 |
| 201893_x_at | DCN | 2.05E−13 | 2.311 | 5.62 | 87.6 | 80.2-92.9 |
| 209335_at | DCN | 2.05E−13 | 2.3115 | 5.62 | 87.6 | 80.3-92.9 |
| 211813_x_at | DCN | 2.05E−13 | 2.3145 | 5.62 | 87.6 | 80.2-92.9 |
| 211896_s_at | DCN | 2.05E−13 | 2.3124 | 5.62 | 87.6 | 80.2-92.9 |
| 204818_at | HSD17B2 | 2.32E−13 | 2.196 | 4.76 | 86.4 | 78.7-92 |
| 204931_at | TCF21 | 2.51E−13 | 2.306 | 2.34 | 87.6 | 80.1-92.9 |
| 204438_at | MRC1 | 2.72E−13 | 2.3105 | 2.6 | 87.6 | 80.2-92.9 |
| 206262_at | ADH1A:ADH1C | 2.94E−13 | 2.3719 | 3.1 | 88.2 | 80.9-93.3 |
| 205433_at | BCHE | 3.18E−13 | 2.2514 | 6.21 | 87 | 79.5-92.5 |
| 225242_s_at | CCDC80 | 3.60E−13 | 2.2316 | 3.67 | 86.8 | 79.2-92.3 |
| 207980_s_at | CITED2 | 5.29E−13 | 2.2709 | 1.64 | 87.2 | 79.7-92.6 |
| 209357_at | CITED2 | 5.29E−13 | 2.2704 | 1.64 | 87.2 | 79.7-92.6 |
| 208383_s_at | PCK1 | 5.74E−13 | 2.3242 | 3.6 | 87.7 | 80.4-93 |
| 206385_s_at | ANK3:LOC729184:LOC731186 | 7.46E−13 | 2.2305 | 2.45 | 86.8 | 79.2-92.2 |
| 203305_at | F13A1 | 8.82E−13 | 2.1821 | 4.74 | 86.2 | 78.6-91.8 |
| 206134_at | ADAMDEC1 | 9.04E−13 | 2.2607 | 3.19 | 87.1 | 79.5-92.5 |
| 215118_s_at | AHNAK:IGHG1 | 9.41E−13 | 2.3277 | 1.73 | 87.8 | 80.3-93 |
| 217022_s_at | AHNAK:IGHG1 | 9.41E−13 | 2.3254 | 1.73 | 87.8 | 80.4-93 |
| 223395_at | ABI3BP | 9.47E−13 | 2.2207 | 3.62 | 86.7 | 79-92.2 |
| 225626_at | PAG1 | 1.11E−12 | 2.2136 | 2.68 | 86.6 | 78.9-92.1 |
| 213953_at | KRT20 | 1.26E−12 | 2.0964 | 5.49 | 85.3 | 77.4-91.1 |
| 226594_at | ENTPD5 | 1.43E−12 | 2.1687 | 3.01 | 86.1 | 78.4-91.8 |
| 209373_at | MALL | 2.10E−12 | 2.1597 | 3.96 | 86 | 78.3-91.7 |
| 212713_at | MFAP4 | 2.96E−12 | 2.1797 | 2.51 | 86.2 | 78.5-91.9 |
| 208920_at | SRI | 3.03E−12 | 2.0982 | 2.43 | 85.3 | 77.4-91.1 |
| 201739_at | SGK | 3.12E−12 | 2.1221 | 4.08 | 85.6 | 77.8-91.3 |
| 214696_at | MGC14376 | 3.39E−12 | 2.1045 | 2.58 | 85.4 | 77.5-91.2 |
| 204034_at | ETHE1 | 3.46E−12 | 2.2127 | 1.75 | 86.6 | 79-92.1 |
| 209667_at | CES2 | 3.51E−12 | 2.102 | 2.87 | 85.3 | 77.4-91.2 |
| 209668_x_at | CES2 | 3.51E−12 | 2.0978 | 2.87 | 85.3 | 77.4-91.1 |
| 213509_x_at | CES2 | 3.51E−12 | 2.097 | 2.87 | 85.3 | 77.4-91.1 |
| 202291_s_at | MGP:C12orf46 | 3.63E−12 | 2.1448 | 4.68 | 85.8 | 78-91.5 |
| 209167_at | GPM6B | 4.04E−12 | 2.1568 | 3.59 | 86 | 78.2-91.6 |
| 209170_s_at | GPM6B | 4.04E−12 | 2.1581 | 3.59 | 86 | 78.2-91.6 |
| 225720_at | SYNPO2 | 5.07E−12 | 2.1053 | 6.43 | 85.4 | 77.5-91.2 |
| 225721_at | SYNPO2 | 5.07E−12 | 2.1069 | 6.43 | 85.4 | 77.5-91.2 |
| 225894_at | SYNPO2 | 5.07E−12 | 2.1043 | 6.43 | 85.4 | 77.5-91.2 |
| 225895_at | SYNPO2 | 5.07E−12 | 2.1069 | 6.43 | 85.4 | 77.5-91.2 |
| 227662_at | SYNPO2 | 5.07E−12 | 2.1043 | 6.43 | 85.4 | 77.5-91.2 |
| 206422_at | GCG | 5.58E−12 | 2.1185 | 12.56 | 85.5 | 77.7-91.3 |
| 205593_s_at | PDE9A | 6.56E−12 | 2.1443 | 3.91 | 85.8 | 78-91.5 |
| 220376_at | LRRC19 | 6.75E−12 | 2.076 | 4.51 | 85 | 77.1-91 |
| 204130_at | HSD11B2 | 6.97E−12 | 2.0896 | 2.53 | 85.2 | 77.3-91.1 |
| 224964_s_at | GNG2 | 7.25E−12 | 2.1405 | 1.91 | 85.8 | 78-91.5 |
| 219508_at | GCNT3 | 8.39E−12 | 2.0843 | 3.94 | 85.1 | 77.2-91 |
| 211645_x_at | No Symbol | 9.19E−12 | 2.1536 | 1.61 | 85.9 | 78.2-91.6 |
| 212233_at | No Symbol | 9.19E−12 | 2.1541 | 1.61 | 85.9 | 78.1-91.6 |
| 212764_at | No Symbol | 9.19E−12 | 2.1541 | 1.61 | 85.9 | 78.1-91.6 |
| 214777_at | No Symbol | 9.19E−12 | 2.1543 | 1.61 | 85.9 | 78.2-91.6 |
| 217235_x_at | No Symbol | 9.19E−12 | 2.155 | 1.61 | 85.9 | 78.2-91.6 |
| 225710_at | No Symbol | 9.19E−12 | 2.1539 | 1.61 | 85.9 | 78.1-91.6 |
| 226333_at | No Symbol | 9.19E−12 | 2.1529 | 1.61 | 85.9 | 78.1-91.6 |
| 226834_at | No Symbol | 9.19E−12 | 2.154 | 1.61 | 85.9 | 78.1-91.6 |
| 227061_at | No Symbol | 9.19E−12 | 2.152 | 1.61 | 85.9 | 78.1-91.6 |
| 228504_at | No Symbol | 9.19E−12 | 2.1533 | 1.61 | 85.9 | 78.1-91.6 |
| 228507_at | No Symbol | 9.19E−12 | 2.1538 | 1.61 | 85.9 | 78.2-91.6 |
| 228640_at | No Symbol | 9.19E−12 | 2.1532 | 1.61 | 85.9 | 78.2-91.6 |
| 228854_at | No Symbol | 9.19E−12 | 2.1532 | 1.61 | 85.9 | 78.2-91.7 |
| 236300_at | No Symbol | 9.19E−12 | 2.1532 | 1.61 | 85.9 | 78.2-91.6 |
| 242317_at | No Symbol | 9.19E−12 | 2.1529 | 1.61 | 85.9 | 78.1-91.6 |
| 210524_x_at | No Symbol | 9.19E−12 | 2.1554 | 1.61 | 85.9 | 78.2-91.7 |
| 224989_at | No Symbol | 9.19E−12 | 2.1526 | 1.61 | 85.9 | 78.2-91.6 |
| 227052_at | No Symbol | 9.19E−12 | 2.151 | 1.61 | 85.9 | 78.1-91.6 |
| 227682_at | No Symbol | 9.19E−12 | 2.1549 | 1.61 | 85.9 | 78.2-91.6 |
| 235146_at | No Symbol | 9.19E−12 | 2.1527 | 1.61 | 85.9 | 78.2-91.7 |
| 207126_x_at | UGT1A10:UGT1A7:UGT1A8: | 9.98E−12 | 2.0677 | 4.56 | 84.9 | 77-90.9 |
| | UGT1A1:UGT1A9:UGT1A6: | | | | | |
| | UGT1A5:UGT1A3:UGT1A4 | | | | | |
| 206094_x_at | UGT1A10:UGT1A7:UGT1A8: | 1.01E−11 | 2.0693 | 4.56 | 85 | 77-90.8 |
| | UGT1A1:UGT1A9:UGT1A6: | | | | | |
| | UGT1A5:UGT1A3:UGT1A4 | | | | | |
| 208596_s_at | UGT1A10:UGT1A7:UGT1A8: | 1.01E−11 | 2.0662 | 4.56 | 84.9 | 76.9-90.8 |
| | UGT1A1:UGT1A9:UGT1A6: | | | | | |
| | UGT1A5:UGT1A3:UGT1A4 | | | | | |
| 221305_s_at | UGT1A10:UGT1A7:UGT1A8: | 1.03E−11 | 2.0663 | 4.56 | 84.9 | 77-90.9 |
| | UGT1A1:UGT1A9:UGT1A6: | | | | | |
| | UGT1A5:UGT1A3:UGT1A4 | | | | | |
| 204532_x_at | UGT1A10:UGT1A7:UGT1A8: | 1.18E−11 | 2.0678 | 4.56 | 84.9 | 77-90.9 |
| | UGT1A1:UGT1A9:UGT1A6: | | | | | |
| | UGT1A5:UGT1A3:UGT1A4 | | | | | |
| 215125_s_at | UGT1A10:UGT1A7:UGT1A8: | 1.18E−11 | 2.0682 | 4.56 | 84.9 | 77-90.9 |
| | UGT1A1:UGT1A9:UGT1A6: | | | | | |
| | UGT1A5:UGT1A3:UGT1A4 | | | | | |
| 209791_at | PADI2 | 1.46E−11 | 2.0258 | 3.98 | 84.4 | 76.4-90.5 |
| 219669_at | CD177 | 1.49E−11 | 2.0618 | 5.77 | 84.9 | 76.9-90.8 |
| 201539_s_at | FHL1 | 1.63E−11 | 2.0448 | 3.25 | 84.7 | 76.7-90.7 |
| 201540_at | FHL1 | 1.63E−11 | 2.0466 | 3.25 | 84.7 | 76.7-90.7 |
| 210298_x_at | FHL1 | 1.63E−11 | 2.0438 | 3.25 | 84.7 | 76.7-90.6 |
| 210299_s_at | FHL1 | 1.63E−11 | 2.0458 | 3.25 | 84.7 | 76.6-90.6 |
| 214505_s_at | FHL1 | 1.63E−11 | 2.0459 | 3.25 | 84.7 | 76.7-90.6 |
| 206576_s_at | CEACAM1 | 2.66E−11 | 2.0448 | 2.85 | 84.7 | 76.7-90.7 |
| 209498_at | CEACAM1 | 2.66E−11 | 2.0446 | 2.85 | 84.7 | 76.7-90.7 |
| 211889_x_at | CEACAM1 | 2.66E−11 | 2.0471 | 2.85 | 84.7 | 76.7-90.7 |
| 202994_s_at | FBLN1 | 2.82E−11 | 2.0183 | 3.55 | 84.4 | 76.3-90.4 |
| 202995_s_at | FBLN1 | 2.82E−11 | 2.0191 | 3.55 | 84.4 | 76.3-90.4 |
| 201427_s_at | SEPP1 | 2.83E−11 | 2.034 | 4.15 | 84.5 | 76.5-90.6 |
| 212956_at | TBC1D9 | 3.89E−11 | 2.1428 | 1.54 | 85.8 | 78-91.5 |
| 203963_at | CA12 | 4.32E−11 | 2.0024 | 2.65 | 84.2 | 76.1-90.2 |
| 204508_s_at | CA12 | 4.32E−11 | 2.0029 | 2.65 | 84.2 | 76.2-90.3 |
| 210735_s_at | CA12 | 4.32E−11 | 2.0016 | 2.65 | 84.2 | 76.1-90.2 |
| 214164_x_at | CA12 | 4.32E−11 | 2.0016 | 2.65 | 84.2 | 76.1-90.3 |
| 215867_x_at | CA12 | 4.32E−11 | 2.0027 | 2.65 | 84.2 | 76.1-90.3 |
| 203881_s_at | DMD | 5.01E−11 | 2.0159 | 4.74 | 84.3 | 76.2-90.4 |
| 213624_at | SMPDL3A | 5.84E−11 | 1.9409 | 3.14 | 83.4 | 75.2-89.6 |
| 226304_at | HSPB6 | 6.16E−11 | 1.9486 | 6.88 | 83.5 | 75.4-89.8 |
| 206561_s_at | LOC441282:AKR1B10:LOC340888 | 7.91E−11 | 1.9478 | 5.17 | 83.5 | 75.3-89.7 |
| 203343_at | UGDH | 8.72E−11 | 1.9619 | 2.4 | 83.7 | 75.6-89.9 |
| 205892_s_at | FABP1 | 9.02E−11 | 1.9694 | 6.54 | 83.8 | 75.6-89.9 |
| 206637_at | P2RY14 | 1.31E−10 | 1.9821 | 2 | 83.9 | 75.8-90.1 |
| 202266_at | TTRAP | 1.59E−10 | 1.9147 | 2.2 | 83.1 | 74.9-89.4 |
| 206000_at | LOC642840:MEP1A:LOC389747: | 2.07E−10 | 1.9873 | 3.17 | 84 | 75.9-90.1 |
| | LOC644777 | | | | | |
| 201496_x_at | MYH11 | 2.15E−10 | 1.8577 | 5.35 | 82.4 | 74-88.8 |
| 201497_x_at | MYH11 | 2.15E−10 | 1.8599 | 5.35 | 82.4 | 74.1-88.8 |
| 207961_x_at | MYH11 | 2.15E−10 | 1.859 | 5.35 | 82.4 | 74.1-88.9 |
| 201495_x_at | MYH11 | 2.15E−10 | 1.8585 | 5.35 | 82.4 | 74-88.8 |
| 204388_s_at | MAOA | 2.49E−10 | 2.0297 | 2.18 | 84.5 | 76.4-90.5 |
| 204389_at | MAOA | 2.49E−10 | 2.0256 | 2.18 | 84.4 | 76.5-90.5 |
| 212741_at | MAOA | 2.49E−10 | 2.0267 | 2.18 | 84.5 | 76.5-90.5 |
| 214598_at | CLDN8 | 2.92E−10 | 1.862 | 12.96 | 82.4 | 74.2-88.9 |
| 202838_at | FUCA1 | 4.24E−10 | 1.8831 | 2.03 | 82.7 | 74.4-89.1 |
| 217897_at | MB:FXYD6 | 6.39E−10 | 1.8109 | 2.92 | 81.7 | 73.3-88.3 |
| 220468_at | ARL14 | 7.01E−10 | 1.8242 | 3.06 | 81.9 | 73.5-88.4 |
| 201920_at | SLC20A1 | 7.65E−10 | 1.7849 | 4.04 | 81.4 | 72.9-88.1 |
| 210302_s_at | MAB21L2 | 1.05E−09 | 1.7744 | 5.2 | 81.3 | 72.8-87.9 |
| 209114_at | TSPAN1 | 1.16E−09 | 1.7896 | 2.16 | 81.5 | 73-88.1 |
| 220266_s_at | KLF4 | 1.19E−09 | 1.8515 | 2.27 | 82.3 | 73.8-88.7 |
| 221841_s_at | KLF4 | 1.19E−09 | 1.8493 | 2.27 | 82.2 | 73.9-88.8 |
| 209283_at | CRYAB | 1.47E−09 | 1.7693 | 3.91 | 81.2 | 72.7-87.8 |
| 223484_at | C15orf48 | 1.54E−09 | 1.8189 | 3.07 | 81.8 | 73.4-88.4 |
| 205412_at | ACAT1 | 1.70E−09 | 1.8575 | 1.77 | 82.3 | 73.9-88.8 |
| 202888_s_at | ANPEP | 2.04E−09 | 1.7673 | 8.18 | 81.2 | 72.6-87.8 |
| 225458_at | EXOC3 | 2.25E−09 | 1.7957 | 3.17 | 81.5 | 73.1-88.1 |
| 204834_at | FGL2 | 2.64E−09 | 1.8245 | 2.07 | 81.9 | 73.4-88.5 |
| 227265_at | FGL2 | 2.64E−09 | 1.8246 | 2.07 | 81.9 | 73.6-88.4 |
| 228469_at | PPID | 3.34E−09 | 1.8032 | 1.73 | 81.6 | 73.2-88.3 |
| 221004_s_at | ITM2C | 3.87E−09 | 1.7063 | 2.22 | 80.3 | 71.8-87.1 |
| 213921_at | SST | 4.16E−09 | 1.7652 | 6.14 | 81.1 | 72.6-87.8 |
| 230087_at | PRIMA1 | 4.74E−09 | 1.7085 | 2.75 | 80.4 | 71.8-87.2 |
| 201842_s_at | EFEMP1 | 5.80E−09 | 1.8196 | 1.69 | 81.9 | 73.4-88.4 |
| 222162_s_at | ADAMTS1 | 6.42E−09 | 1.7467 | 2.37 | 80.9 | 72.3-87.6 |
| 210517_s_at | AKAP12 | 7.41E−09 | 1.7454 | 2.83 | 80.9 | 72.3-87.6 |
| 227529_s_at | AKAP12 | 7.41E−09 | 1.7437 | 2.83 | 80.8 | 72.3-87.6 |
| 228750_at | COL14A1 | 7.60E−09 | 1.7813 | 1.67 | 81.3 | 72.9-88 |
| 219948_x_at | LOC642329:UGT2A3 | 8.64E−09 | 1.7422 | 4.89 | 80.8 | 72.2-87.5 |
| 208131_s_at | PTGIS | 9.15E−09 | 1.7212 | 3.98 | 80.5 | 71.9-87.3 |
| 207432_at | BTN2A2:BEST2 | 9.19E−09 | 1.7179 | 3.63 | 80.5 | 71.9-87.3 |
| 219607_s_at | MS4A4A:LOC643680 | 9.59E−09 | 1.8197 | 1.55 | 81.9 | 73.4-88.4 |
| 204688_at | SGCE | 1.02E−08 | 1.7438 | 2.03 | 80.8 | 72.3-87.6 |
| 207134_x_at | TPSB2 | 1.24E−08 | 1.3713 | 1.87 | 75.4 | 66.3-82.9 |
| 209156_s_at | COL6A2 | 1.30E−08 | 1.68 | 2.08 | 80 | 71.3-86.8 |
| 202741_at | PRKACB | 1.30E−08 | 1.7217 | 2.18 | 80.5 | 71.9-87.3 |
| 202742_s_at | PRKACB | 1.30E−08 | 1.721 | 2.18 | 80.5 | 71.9-87.3 |
| 200795_at | SPARCL1 | 1.33E−08 | 1.6379 | 2.85 | 79.4 | 70.7-86.3 |
| 219543_at | PBLD | 1.38E−08 | 1.6981 | 2.74 | 80.2 | 71.6-87.1 |
| 225207_at | PDK4 | 1.45E−08 | 1.705 | 3.02 | 80.3 | 71.7-87.2 |
| 202222_s_at | SPEG:LOC729871:DES | 1.47E−08 | 1.7229 | 6.66 | 80.6 | 72-87.3 |
| 214027_x_at | SPEG:LOC729871:DES | 1.47E−08 | 1.7254 | 6.66 | 80.6 | 72-87.4 |
| 239272_at | MMP28 | 1.80E−08 | 1.6622 | 2.47 | 79.7 | 71-86.6 |
| 222453_at | THEM4:CYBRD1 | 1.84E−08 | 1.6934 | 2.7 | 80.1 | 71.6-87 |
| 212195_at | MAGEA4:IL6ST | 1.87E−08 | 1.764 | 1.58 | 81.1 | 72.6-87.8 |
| 203980_at | FABP4 | 2.01E−08 | 1.6762 | 5.55 | 79.9 | 71.3-86.8 |
| 211985_s_at | CALM1 | 2.12E−08 | 1.6839 | 1.72 | 80 | 71.4-86.9 |
| 221747_at | TNS1:AKAP9 | 2.21E−08 | 1.7434 | 2.01 | 80.8 | 72.2-87.5 |
| 221748_s_at | TNS1:AKAP9 | 2.21E−08 | 1.7409 | 2.01 | 80.8 | 72.3-87.5 |
| 201324_at | EMP1 | 2.59E−08 | 1.7149 | 1.95 | 80.4 | 71.9-87.3 |
| 212397_at | LOC643244:RDX | 3.06E−08 | 1.7431 | 1.9 | 80.8 | 72.3-87.6 |
| 205382_s_at | CFD | 3.06E−08 | 1.6462 | 2.81 | 79.5 | 70.8-86.4 |
| 201141_at | GPNMB | 3.11E−08 | 1.6928 | 1.99 | 80.1 | 71.5-87 |
| 205683_x_at | TPSAB1, TPSAB1 | 3.52E−08 | 1.7075 | 1.93 | 80.3 | 71.8-87.1 |
| 210084_x_at | TPSAB1, TPSAB1 | 3.52E−08 | 1.7045 | 1.93 | 80.3 | 71.8-87.1 |
| 215382_x_at | TPSAB1, TPSAB1 | 3.52E−08 | 1.7091 | 1.93 | 80.4 | 71.7-87.2 |
| 216474_x_at | TPSAB1, TPSAB1 | 3.52E−08 | 1.7069 | 1.93 | 80.3 | 71.7-87.2 |
| 227006_at | PPP1R14A | 3.54E−08 | 1.619 | 2.51 | 79.1 | 70.3-86.1 |
| 242601_at | LOC253012 | 3.59E−08 | 1.6517 | 4.46 | 79.6 | 70.8-86.5 |
| 212730_at | DMN | 4.36E−08 | 1.6046 | 4.05 | 78.9 | 70.2-86 |
| 226818_at | MPEG1 | 4.58E−08 | 1.6466 | 1.86 | 79.5 | 70.8-86.5 |
| 226841_at | MPEG1 | 4.58E−08 | 1.6484 | 1.86 | 79.5 | 70.8-86.5 |
| 203474_at | IQGAP2 | 5.06E−08 | 1.6275 | 2.51 | 79.2 | 70.6-86.2 |
| 203766_s_at | LMOD1 | 5.55E−08 | 1.58 | 2.97 | 78.5 | 69.8-85.6 |
| 214916_x_at | LOC652128:IGHG1:IGHM:IGHV4- | 5.75E−08 | 1.6352 | 3.1 | 79.3 | 70.6-86.3 |
| | 31:LOC647189:IGHV1- | | | | | |
| | 69:IGHA1:IL8:EXOC7:SIX6:IGHD: | | | | | |
| | IGH@:IGHG3:C12orf32: | | | | | |
| | ZCWPW2:IFI6:IGHG4:IGHA2: | | | | | |
| | IGHG2:RAC1 | | | | | |
| 226303_at | PGM5 | 5.87E−08 | 1.6215 | 5.87 | 79.1 | 70.4-86.2 |
| 225442_at | DDR2 | 5.89E−08 | 1.562 | 2.61 | 78.3 | 69.4-85.4 |
| 227561_at | DDR2 | 5.89E−08 | 1.5604 | 2.61 | 78.2 | 69.4-85.5 |
| 202760_s_at | AKAP2:PALM2:PALM2- | 6.28E−08 | 1.6137 | 2.17 | 79 | 70.3-86.1 |
| | AKAP2 | | | | | |
| 226694_at | AKAP2:PALM2:PALM2- | 6.28E−08 | 1.6143 | 2.17 | 79 | 70.2-86.1 |
| | AKAP2 | | | | | |
| 204894_s_at | AOC3 | 6.43E−08 | 1.556 | 2.92 | 78.2 | 69.4-85.4 |
| 203058_s_at | PAPSS2 | 6.43E−08 | 1.6171 | 1.97 | 79.1 | 70.4-86.1 |
| 203060_s_at | PAPSS2 | 6.43E−08 | 1.6159 | 1.97 | 79 | 70.4-86.1 |
| 208763_s_at | TSC22D3 | 7.22E−08 | 1.6098 | 2.76 | 79 | 70.2-86 |
| 222717_at | SDPR | 9.62E−08 | 1.5998 | 2.21 | 78.8 | 70.1-85.9 |
| 203680_at | PRKAR2B | 1.05E−07 | 1.6759 | 2.1 | 79.9 | 71.2-86.8 |
| 201041_s_at | DUSP1 | 1.06E−07 | 1.5295 | 2.44 | 77.8 | 68.9-85 |
| 209374_s_at | LOC652128:IGHG1:IGHM:IGHV4- | 1.12E−07 | 1.6333 | 3.1 | 79.3 | 70.6-86.3 |
| | 31:LOC647189:IGHV1- | | | | | |
| | 69:IGHA1:IL8:EXOC7:SIX6:IGHD: | | | | | |
| | IGH@:IGHG3:C12orf32: | | | | | |
| | ZCWPW2:IFI6:IGHG4:IGHA2: | | | | | |
| | IGHG2:RAC1 | | | | | |
| 216491_x_at | LOC652128:IGHG1:IGHM:IGHV4- | 1.12E−07 | 1.6335 | 3.1 | 79.3 | 70.5-86.3 |
| | 31:LOC647189:IGHV1- | | | | | |
| | 69:IGHA1:IL8:EXOC7:SIX6:IGHD: | | | | | |
| | IGH@:IGHG3:C12orf32: | | | | | |
| | ZCWPW2:IFI6:IGHG4:IGHA2: | | | | | |
| | IGHG2:RAC1 | | | | | |
| 229831_at | CNTN3 | 1.20E−07 | 1.6157 | 3.93 | 79 | 70.2-86.1 |
| 235766_x_at | RAB27A | 1.22E−07 | 1.5984 | 1.79 | 78.8 | 70.1-85.9 |
| 229070_at | C6orf105 | 1.38E−07 | 1.6049 | 2.13 | 78.9 | 70.1-86 |
| 226654_at | MUC12 | 1.53E−07 | 1.4559 | 4.89 | 76.7 | 67.7-84.1 |
| 202686_s_at | AXL | 1.59E−07 | 1.5758 | 1.71 | 78.5 | 69.7-85.6 |
| 205403_at | IL1R2 | 1.73E−07 | 1.5237 | 2.54 | 77.7 | 68.8-84.9 |
| 211372_s_at | IL1R2 | 1.73E−07 | 1.5229 | 2.54 | 77.7 | 68.7-85 |
| 205929_at | GPA33 | 1.86E−07 | 1.7056 | 1.61 | 80.3 | 71.7-87.2 |
| 202069_s_at | IDH3A | 2.08E−07 | 1.5696 | 1.75 | 78.4 | 69.5-85.5 |
| 202350_s_at | MATN2 | 2.11E−07 | 1.506 | 2.23 | 77.4 | 68.4-84.7 |
| 212859_x_at | NUTF2:MT1E:MT1M | 2.14E−07 | 1.6442 | 2.41 | 79.4 | 70.8-86.4 |
| 217546_at | NUTF2:MT1E:MT1M | 2.14E−07 | 1.645 | 2.41 | 79.5 | 70.7-86.5 |
| 216336_x_at | NUTF2:MT1E:MT1M | 2.14E−07 | 1.645 | 2.41 | 79.5 | 70.8-86.4 |
| 221667_s_at | HSPB8 | 2.61E−07 | 1.514 | 3.85 | 77.5 | 68.6-84.8 |
| 217757_at | A2M | 2.72E−07 | 1.598 | 1.64 | 78.8 | 70.1-85.9 |
| 216510_x_at | LOC652128:IGHG1:IGHM:IGHV4- | 2.80E−07 | 1.6346 | 3.1 | 79.3 | 70.6-86.4 |
| | 31:LOC647189:IGHV1- | | | | | |
| | 69:IGHA1:IL8:EXOC7:SIX6:IGHD: | | | | | |
| | IGH@:IGHG3:C12orf32: | | | | | |
| | ZCWPW2:IFI6:IGHG4:IGHA2: | | | | | |
| | IGHG2:RAC1 | | | | | |
| 223343_at | NYD-SP21:MS4A7 | 2.92E−07 | 1.5396 | 2.27 | 77.9 | 69.1-85.1 |
| 202620_s_at | PLOD2 | 3.06E−07 | 1.5551 | 2.33 | 78.2 | 69.3-85.4 |
| 207245_at | UGT2B17 | 3.75E−07 | 1.4796 | 4.88 | 77 | 68.1-84.5 |
| 210139_s_at | PMP22 | 3.97E−07 | 1.5183 | 2.23 | 77.6 | 68.7-84.9 |
| 204938_s_at | PLN | 4.14E−07 | 1.5454 | 2.01 | 78 | 69.1-85.2 |
| 204939_s_at | PLN | 4.14E−07 | 1.5425 | 2.01 | 78 | 69.1-85.2 |
| 204940_at | PLN | 4.14E−07 | 1.5436 | 2.01 | 78 | 69.1-85.2 |
| 203951_at | CNN1 | 4.77E−07 | 1.4324 | 4.56 | 76.3 | 67.3-83.8 |
| 202746_at | ITM2A | 5.27E−07 | 1.4988 | 2.77 | 77.3 | 68.4-84.6 |
| 221584_s_at | KCNMA1 | 6.86E−07 | 1.4959 | 2.37 | 77.3 | 68.4-84.6 |
| 241994_at | XDH | 8.13E−07 | 1.4709 | 2.03 | 76.9 | 67.9-84.3 |
| 209621_s_at | PDLIM3 | 8.20E−07 | 1.4805 | 2.38 | 77 | 68-84.4 |
| 204326_x_at | LOC645652:MT1X | 9.84E−07 | 1.4427 | 4.35 | 76.5 | 67.4-83.9 |
| 208581_x_at | LOC645652:MT1X | 9.84E−07 | 1.4397 | 4.35 | 76.4 | 67.5-83.9 |
| 201616_s_at | CALD1 | 1.02E−06 | 1.4583 | 3.29 | 76.7 | 67.8-84.1 |
| 201617_x_at | CALD1 | 1.02E−06 | 1.4617 | 3.29 | 76.8 | 67.7-84.1 |
| 212077_at | CALD1 | 1.02E−06 | 1.4581 | 3.29 | 76.7 | 67.8-84.1 |
| 225782_at | MSRB3 | 1.20E−06 | 1.4747 | 2.24 | 77 | 68-84.3 |
| 209436_at | SPON1 | 1.46E−06 | 1.4849 | 1.88 | 77.1 | 68.1-84.4 |
| 223623_at | C2orf40 | 1.69E−06 | 1.416 | 2.97 | 76.1 | 67-83.5 |
| 218087_s_at | SORBS1:KIAA0894 | 2.18E−06 | 1.4259 | 2.54 | 76.2 | 67.3-83.7 |
| 222513_s_at | SORBS1:KIAA0894 | 2.18E−06 | 1.4263 | 2.54 | 76.2 | 67.2-83.7 |
| 224990_at | C4orf34 | 2.55E−06 | 1.3853 | 1.95 | 75.6 | 66.5-83.1 |
| 203131_at | FIP1L1:PDGFRA | 2.94E−06 | 1.4845 | 1.37 | 77.1 | 68.2-84.5 |
| 203638_s_at | FGFR2 | 3.55E−06 | 1.5287 | 1.31 | 77.8 | 68.9-85 |
| 201957_at | PPP1R12B:LOC731632 | 4.26E−06 | 1.3611 | 2.21 | 75.2 | 66.2-82.8 |
| 217967_s_at | FAM129A | 4.59E−06 | 1.4131 | 1.8 | 76 | 67-83.5 |
| 210809_s_at | POSTN | 4.94E−06 | 1.3954 | 2.25 | 75.7 | 66.7-83.2 |
| 226302_at | ATP8B1 | 5.73E−06 | 1.313 | 1.67 | 74.4 | 65.3-82.1 |
| 238750_at | CCL28 | 6.01E−06 | 1.4932 | 1.53 | 77.2 | 68.3-84.6 |
| 226103_at | NEXN | 6.83E−06 | 1.3666 | 2.23 | 75.3 | 66.2-82.8 |
| 209101_at | CTGF | 6.96E−06 | 1.4774 | 1.3 | 77 | 68.1-84.4 |
| 229254_at | MFSD4 | 7.19E−06 | 1.3235 | 2.12 | 74.6 | 65.4-82.3 |
| 219087_at | ASPN | 7.45E−06 | 1.3249 | 2.74 | 74.6 | 65.5-82.3 |
| 209457_at | DUSP5 | 8.60E−06 | 1.3336 | 1.91 | 74.8 | 65.7-82.4 |
| 221541_at | CRISPLD2 | 9.24E−06 | 1.3664 | 1.64 | 75.3 | 66.2-82.9 |
| 206377_at | FOXF2 | 9.50E−06 | 1.3032 | 2.08 | 74.3 | 65.1-82 |
| 207392_x_at | UGT2B11:LOC728160:UGT2B15, | 9.73E−06 | 1.3478 | 5.72 | 75 | 65.8-82.6 |
| | UGT2B11:LOC728160:UGT2B15 | | | | | |
| 202274_at | ACTG2 | 1.07E−05 | 1.4015 | 1.82 | 75.8 | 66.8-83.4 |
| 227522_at | CMBL | 1.09E−05 | 1.3588 | 1.58 | 75.2 | 66-82.8 |
| 212192_at | KCTD12 | 1.44E−05 | 1.3659 | 1.36 | 75.3 | 66.2-82.8 |
| 227727_at | MRGPRF | 1.52E−05 | 1.2965 | 2.99 | 74.2 | 65-82 |
| 234987_at | C20orf118 | 1.75E−05 | 1.4278 | 1.3 | 76.2 | 67.3-83.7 |
| 209656_s_at | TMEM47 | 1.86E−05 | 1.2983 | 2.02 | 74.2 | 65.1-82 |
| 212265_at | QKI | 1.93E−05 | 1.3443 | 1.31 | 74.9 | 65.9-82.6 |
| 228232_s_at | VSIG2 | 2.01E−05 | 1.2957 | 2.05 | 74.1 | 65-81.9 |
| 200799_at | HSPA1A, HSPA1A | 2.16E−05 | 1.272 | 2.41 | 73.8 | 64.6-81.6 |
| 218312_s_at | ZNF447 | 3.05E−05 | 1.107 | 1.39 | 71 | 61.6-79.1 |
| 230264_s_at | AP1S2 | 3.39E−05 | 1.2546 | 1.92 | 73.5 | 64.3-81.4 |
| 224840_at | FKBP5 | 3.41E−05 | 1.2371 | 1.71 | 73.2 | 63.9-81.1 |
| 209948_at | KCNMB1 | 3.74E−05 | 1.2181 | 2.66 | 72.9 | 63.6-80.8 |
| 201426_s_at | VIM | 4.15E−05 | 1.2221 | 1.86 | 72.9 | 63.7-80.8 |
| 227826_s_at | SORBS2 | 4.23E−05 | 1.3017 | 2.1 | 74.2 | 65.1-82 |
| 227827_at | SORBS2 | 4.23E−05 | 1.3015 | 2.1 | 74.2 | 65.1-82 |
| 225728_at | SORBS2 | 4.23E−05 | 1.3015 | 2.1 | 74.2 | 65.1-82 |
| 238751_at | SORBS2 | 4.23E−05 | 1.3019 | 2.1 | 74.2 | 65.1-82 |
| 228202_at | C6orf204 | 4.90E−05 | 1.2062 | 2.07 | 72.7 | 63.5-80.6 |
| 220645_at | FAM55D | 5.67E−05 | 1.1786 | 3.48 | 72.2 | 63-80.2 |
| 224560_at | TIMP2 | 6.40E−05 | 1.2304 | 1.82 | 73.1 | 63.9-80.9 |
| 231579_s_at | TIMP2 | 6.40E−05 | 1.2286 | 1.82 | 73 | 63.8-81 |
| 208788_at | ELOVL5 | 8.15E−05 | 1.1804 | 2.05 | 72.2 | 63-80.2 |
| 200974_at | ACTA2 | 1.00E−04 | 1.2299 | 1.81 | 73.1 | 63.8-81 |
| 202388_at | RGS2 | 0.0001 | 1.1604 | 1.94 | 71.9 | 62.7-80 |
| 206461_x_at | MT1A:MT1H:LOC645745:LOC727730 | 0.0001 | 1.189 | 4.55 | 72.4 | 63.2-80.4 |
| 208747_s_at | C1S:PRB1:PRB2, C1S:PRB2:PRB1 | 0.0001 | 1.2884 | 1.61 | 74 | 64.9-81.9 |
| 208791_at | CLU | 0.0001 | 1.2303 | 1.42 | 73.1 | 63.9-80.9 |
| 208792_s_at | CLU | 0.0001 | 1.2302 | 1.42 | 73.1 | 63.9-81 |
| 212185_x_at | NUTF2:LOC441019:MT2A:MT1M, | 0.0001 | 1.1894 | 1.89 | 72.4 | 63.1-80.3 |
| | NUTF2:LOC441019:MT2A: | | | | | |
| | MT1M | | | | | |
| 222043_at | CLU | 0.0001 | 1.2306 | 1.42 | 73.1 | 63.9-81 |
| 227099_s_at | LOC387763 | 0.0001 | 1.1963 | 1.36 | 72.5 | 63.2-80.5 |
| 202766_s_at | FBN1 | 0.0002 | 1.1359 | 1.93 | 71.5 | 62.2-79.6 |
| 204745_x_at | NUTF2:MT1G | 0.0002 | 1.0888 | 2.66 | 70.7 | 61.3-78.9 |
| 218418_s_at | ANKRD25 | 0.0002 | 1.1139 | 1.56 | 71.1 | 61.9-79.3 |
| 201058_s_at | MYL9 | 0.0003 | 1.131 | 2.18 | 71.4 | 62.1-79.5 |
| 205547_s_at | TAGLN | 0.0003 | 1.0914 | 2.12 | 70.7 | 61.4-78.9 |
| 205935_at | FOXF1 | 0.0004 | 1.1743 | 1.28 | 72.1 | 62.9-80.1 |
| 208450_at | LGALS2 | 0.0004 | 1.0845 | 3.07 | 70.6 | 61.4-78.8 |
| 203748_x_at | RBMS1 | 0.0005 | 1.1435 | 1.55 | 71.6 | 62.3-79.7 |
| 207266_x_at | RBMS1 | 0.0005 | 1.1428 | 1.55 | 71.6 | 62.4-79.7 |
| 209868_s_at | RBMS1 | 0.0005 | 1.1437 | 1.55 | 71.6 | 62.3-79.7 |
| 225269_s_at | RBMS1 | 0.0005 | 1.1446 | 1.55 | 71.6 | 62.3-79.6 |
| 212158_at | SDC2 | 0.0006 | 1.0977 | 1.39 | 70.8 | 61.6-79.1 |
| 209209_s_at | PLEKHC1 | 0.0007 | 1.0309 | 1.82 | 69.7 | 60.3-77.9 |
| 209210_s_at | PLEKHC1 | 0.0007 | 1.0325 | 1.82 | 69.7 | 60.3-78 |
| 205554_s_at | ACTB:DNASE1L3 | 0.0007 | 1.0979 | 1.5 | 70.8 | 61.4-78.9 |
| 202283_at | SERPINF1 | 0.0009 | 1.0003 | 1.47 | 69.2 | 59.8-77.5 |
| 209496_at | RARRES2:LOC648925 | 0.0009 | 1.0318 | 1.6 | 69.7 | 60.3-77.9 |
| 224352_s_at | CFL2 | 0.0009 | 1.0586 | 1.75 | 70.2 | 60.8-78.4 |
| 224663_s_at | CFL2 | 0.0009 | 1.0575 | 1.75 | 70.2 | 60.8-78.4 |
| 214433_s_at | SELENBP1 | 0.0009 | 1.0574 | 1.7 | 70.1 | 60.8-78.3 |
| 212592_at | ENAM | 0.0011 | 1.0024 | 2.58 | 69.2 | 59.8-77.5 |
| 213629_x_at | MT1F | 0.0012 | 0.9664 | 2.2 | 68.6 | 59.2-76.9 |
| 217165_x_at | MT1F | 0.0012 | 0.9649 | 2.2 | 68.5 | 59.2-76.9 |
| 203645_s_at | CD163 | 0.0013 | 1.0231 | 1.9 | 69.6 | 60-77.9 |
| 215049_x_at | CD163 | 0.0013 | 1.0215 | 1.9 | 69.5 | 60.1-77.8 |
| 206641_at | TNFRSF17 | 0.0015 | 1.0206 | 2.08 | 69.5 | 60.1-77.8 |
| 227235_at | GUCY1A3 | 0.0015 | 1.0116 | 2.69 | 69.4 | 59.8-77.6 |
| 229530_at | GUCY1A3 | 0.0015 | 1.0115 | 2.69 | 69.3 | 60-77.6 |
| 212097_at | CAV1 | 0.0016 | 1.0199 | 1.58 | 69.5 | 60.1-77.7 |
| 200884_at | CKB | 0.0019 | 0.9758 | 2.27 | 68.7 | 59.2-77 |
| 202133_at | WWTR1 | 0.002 | 0.9917 | 1.48 | 69 | 59.5-77.4 |
| 204607_at | HMGCS2 | 0.0022 | 0.8777 | 2.67 | 67 | 57.5-75.5 |
| 218559_s_at | MAFB | 0.0034 | 0.9595 | 1.66 | 68.4 | 59-76.8 |
| 201061_s_at | STOM | 0.0035 | 1.0165 | 1.21 | 69.4 | 60-77.7 |
| 201743_at | CD14 | 0.0035 | 0.9627 | 1.58 | 68.5 | 59.1-76.9 |
| 204895_x_at | MUC4:TAF5L:LOC650855:LOC645744 | 0.0039 | 1.0448 | 1.2 | 69.9 | 60.6-78.2 |
| 217109_at | MUC4:TAF5L:LOC650855:LOC645744 | 0.0039 | 1.0458 | 1.2 | 69.9 | 60.6-78.1 |
| 217110_s_at | MUC4:TAF5L:LOC650855:LOC645744 | 0.0039 | 1.0453 | 1.2 | 69.9 | 60.5-78.2 |
| 203382_s_at | APOE | 0.0044 | 0.9388 | 1.56 | 68.1 | 58.7-76.5 |
| 206664_at | SI | 0.0054 | 0.9293 | 2.79 | 67.9 | 58.4-76.3 |
| 242447_at | LOC285382 | 0.0058 | 0.7401 | 1.26 | 64.4 | 54.9-73.2 |
| 209312_x_at | HLA-DRB1:HLA- | 0.0063 | 1.0636 | 1.51 | 70.3 | 60.9-78.5 |
| | DRB6:LOC731247:LOC730415: | | | | | |
| | HLA-DRB4:HLA- | | | | | |
| | DRB5:HLA- | | | | | |
| | DRB3:LOC651845 | | | | | |
| 215193_x_at | HLA-DRB1:HLA- | 0.0063 | 1.0646 | 1.51 | 70.3 | 60.9-78.5 |
| | DRB6:LOC731247:LOC730415: | | | | | |
| | HLA-DRB4:HLA- | | | | | |
| | DRB5:HLA- | | | | | |
| | DRB3:LOC651845 | | | | | |
| 224694_at | ANTXR1 | 0.0071 | 0.9531 | 1.29 | 68.3 | 58.8-76.7 |
| 226811_at | FAM46C | 0.0085 | 0.9163 | 1.38 | 67.7 | 58.1-76.1 |
| 201150_s_at | TIMP3 | 0.0091 | 0.9049 | 1.81 | 67.5 | 57.9-76 |
| 226682_at | LOC283666 | 0.0099 | 0.7784 | 1.22 | 65.1 | 55.7-73.8 |
| 218468_s_at | GREM1 | 0.0106 | 0.8585 | 3.03 | 66.6 | 57.2-75.2 |
| 218469_at | GREM1 | 0.0106 | 0.8579 | 3.03 | 66.6 | 57.1-75.2 |
| 203240_at | FCGBP:LOC651441:LOC652599 | 0.0121 | 0.7868 | 1.99 | 65.3 | 55.8-73.9 |
| 211538_s_at | HSPA2 | 0.0123 | 0.8916 | 1.43 | 67.2 | 57.7-75.7 |
| 212091_s_at | COL6A1 | 0.0126 | 0.848 | 1.45 | 66.4 | 56.9-74.9 |
| 213428_s_at | COL6A1 | 0.0126 | 0.8474 | 1.45 | 66.4 | 56.9-74.9 |
| 226051_at | SELM | 0.0133 | 0.8606 | 1.32 | 66.7 | 57.1-75.1 |
| 200859_x_at | FLNA:WTAP | 0.0157 | 0.7975 | 1.59 | 65.5 | 55.9-74 |
| 213746_s_at | FLNA:WTAP | 0.0157 | 0.7959 | 1.59 | 65.5 | 56-74.2 |
| 214752_x_at | FLNA:WTAP | 0.0157 | 0.798 | 1.59 | 65.5 | 56-74.1 |
| 204570_at | COX7A1 | 0.0161 | 0.8622 | 1.32 | 66.7 | 57.2-75.2 |
| 203729_at | EMP3 | 0.0164 | 0.8043 | 1.41 | 65.6 | 56.1-74.3 |
| 223597_at | ITLN1 | 0.021 | 0.7148 | 2.54 | 64 | 54.4-72.7 |
| 227735_s_at | C10orf99 | 0.0218 | 0.7254 | 1.76 | 64.2 | 54.6-72.9 |
| 227736_at | C10orf99 | 0.0218 | 0.7259 | 1.76 | 64.2 | 54.6-72.9 |
| 200621_at | CSRP1 | 0.023 | 0.7246 | 1.39 | 64.1 | 54.6-72.9 |
| 204897_at | PTGER4:LOC730002:LOC730882 | 0.0237 | 0.8114 | 1.41 | 65.8 | 56.2-74.4 |
| 204083_s_at | PPIL5:TPM2 | 0.026 | 0.7531 | 1.53 | 64.7 | 55.2-73.4 |
| 211643_x_at | HLA-C:HLA- | 0.0268 | 0.9237 | 1.24 | 67.8 | 58.3-76.3 |
| | B:LOC730410:LOC652614:LOC732037 | | | | | |
| 211644_x_at | HLA-C:HLA- | 0.0268 | 0.9229 | 1.24 | 67.8 | 58.3-76.2 |
| | B:LOC730410:LOC652614:LOC732037 | | | | | |
| 214768_x_at | HLA-C:HLA- | 0.0268 | 0.9245 | 1.24 | 67.8 | 58.3-76.3 |
| | B:LOC730410:LOC652614:LOC732037 | | | | | |
| 226147_s_at | PIGR | 0.0278 | 0.7282 | 1.59 | 64.2 | 54.6-73 |
| 229659_s_at | PIGR | 0.0278 | 0.7253 | 1.59 | 64.2 | 54.7-72.9 |
| 201300_s_at | PRNP | 0.0288 | 0.7985 | 1.24 | 65.5 | 56-74.1 |
| 210133_at | CCL11 | 0.0361 | 0.8534 | 1.32 | 66.5 | 57-75.1 |
| 225353_s_at | C1QC | 0.0391 | 0.7457 | 1.36 | 64.5 | 55-73.2 |
| 201289_at | CYR61 | 0.0425 | 0.7849 | 1.47 | 65.3 | 55.8-73.9 |
| 210764_s_at | CYR61 | 0.0425 | 0.7836 | 1.47 | 65.2 | 55.6-73.9 |
| 200986_at | SERPING1 | 0.0503 | 0.7672 | 1.44 | 64.9 | 55.4-73.6 |
| 204122_at | TYROBP:ZNF160 | 0.053 | 0.8181 | 1.17 | 65.9 | 56.4-74.5 |
| 201667_at | GJA1 | 0.0561 | 0.784 | 1.76 | 65.2 | 55.7-73.9 |
| 208789_at | PTRF | 0.0624 | 0.736 | 1.5 | 64.4 | 54.8-73.1 |
| 210107_at | CLCA1 | 0.0681 | 0.7868 | 1.61 | 65.3 | 55.7-73.9 |
| 200665_s_at | SPARC | 0.0684 | 0.6714 | 1.45 | 63.1 | 53.6-72.1 |
| 212667_at | SPARC | 0.0684 | 0.6732 | 1.45 | 63.2 | 53.6-71.9 |
| 211964_at | COL4A2 | 0.0738 | 0.7064 | 1.32 | 63.8 | 54.2-72.6 |
| 201858_s_at | PRG1 | 0.0972 | 0.5559 | 1.25 | 60.9 | 51.3-69.9 |
| 201859_at | PRG1 | 0.0972 | 0.5563 | 1.25 | 61 | 51.4-69.9 |
| 228241_at | BCMP11 | 0.102 | 0.4028 | 1.28 | 58 | 48.4-67.1 |
| 217762_s_at | RAB31 | 0.1041 | 0.8135 | 1.14 | 65.8 | 56.3-74.4 |
| 217764_s_at | RAB31 | 0.1041 | 0.8132 | 1.14 | 65.8 | 56.2-74.5 |
| 202007_at | NID1 | 0.105 | 0.7425 | 1.33 | 64.5 | 55-73.1 |
| 210495_x_at | FN1 | 0.1121 | 0.5756 | 1.73 | 61.3 | 51.8-70.2 |
| 211719_x_at | FN1 | 0.1121 | 0.5767 | 1.73 | 61.3 | 51.8-70.2 |
| 212464_s_at | FN1 | 0.1121 | 0.5765 | 1.73 | 61.3 | 51.6-70.3 |
| 216442_x_at | FN1 | 0.1121 | 0.5745 | 1.73 | 61.3 | 51.7-70.3 |
| 200600_at | MSN | 0.1499 | 0.7232 | 1.15 | 64.1 | 54.6-72.9 |
| 209138_x_at | IGL@:LOC651536:LOC731062: | 0.1551 | 0.681 | 1.26 | 63.3 | 53.8-72.2 |
| | CPVL:IGLV2-14:IGLV4- | | | | | |
| | 3:IGLV3-25:IGLV3- | | | | | |
| | 21:IL8:RPL14 | | | | | |
| 216984_x_at | IGL@:LOC651536:LOC731062: | 0.1551 | 0.6808 | 1.26 | 63.3 | 53.8-72.2 |
| | CPVL:IGLV2-14:IGLV4- | | | | | |
| | 3:IGLV3-25:IGLV3- | | | | | |
| | 21:IL8:RPL14 | | | | | |
| 217148_x_at | IGL@:LOC651536:LOC731062: | 0.1551 | 0.6808 | 1.26 | 63.3 | 53.9-72.1 |
| | CPVL:IGLV2-14:IGLV4- | | | | | |
| | 3:IGLV3-25:IGLV3- | | | | | |
| | 21:IL8:RPL14 | | | | | |
| 201069_at | MMP2 | 0.1755 | 0.6214 | 1.24 | 62.2 | 52.6-71 |
| 202403_s_at | COL1A2:LOC728628 | 0.1891 | 0.6508 | 1.9 | 62.8 | 53.2-71.5 |
| 202768_at | FOSB | 0.2076 | 0.6392 | 2.17 | 62.5 | 52.9-71.5 |
| 209116_x_at | HBB | 0.2357 | 0.6432 | 1.28 | 62.6 | 52.9-71.4 |
| 211696_x_at | HBB | 0.2357 | 0.6436 | 1.28 | 62.6 | 53.1-71.4 |
| 217232_x_at | HBB | 0.2357 | 0.6444 | 1.28 | 62.6 | 53.1-71.5 |
| 205267_at | POU2AF1 | 0.2389 | 0.6573 | 1.18 | 62.9 | 53.3-71.7 |
| 201852_x_at | COL3A1 | 0.2551 | 0.5524 | 1.35 | 60.9 | 51.3-69.8 |
| 211161_s_at | COL3A1 | 0.2551 | 0.5509 | 1.35 | 60.9 | 51.3-69.9 |
| 215076_s_at | COL3A1 | 0.2551 | 0.5513 | 1.35 | 60.9 | 51.3-69.8 |
| 217378_x_at | LOC391427 | 0.2627 | 0.2759 | 1.15 | 55.5 | 45.9-64.8 |
| 223235_s_at | SMOC2 | 0.2701 | 0.656 | 1.34 | 62.9 | 53.2-71.7 |
| 204673_at | MUC5AC:LOC652741:MUC2 | 0.2802 | 0.8531 | 1.22 | 66.5 | 57-75.1 |
| 212414_s_at | Sep-06 | 0.3109 | 0.5714 | 1.21 | 61.2 | 51.7-70.2 |
| 201744_s_at | LUM | 0.3157 | 0.5425 | 1.54 | 60.7 | 51-69.7 |
| 202953_at | C1QB | 0.3668 | 0.4998 | 1.24 | 59.9 | 50.3-68.9 |
| 209651_at | TGFB1I1 | 0.4093 | 0.4932 | 1.35 | 59.7 | 50.2-68.8 |
| 212224_at | ALDH1A1 | 0.4232 | 0.3525 | 1.4 | 57 | 47.4-66.2 |
| 211596_s_at | LRIG1 | 0.4614 | 0.5988 | 1.26 | 61.8 | 52.2-70.7 |
| 218541_s_at | C8orf4 | 0.475 | 0.4838 | 1.44 | 59.6 | 50-68.5 |
| 208894_at | HLA-DRA:HLA-DQA1, HLA- | 0.4823 | 0.3234 | 1.29 | 56.4 | 46.8-65.6 |
| | DRA:HLA-DQA1, HLA- | | | | | |
| | DRA:HLA-DQA1 | | | | | |
| 210982_s_at | HLA-DRA:HLA-DQA1, HLA- | 0.4823 | 0.3228 | 1.29 | 56.4 | 46.8-65.6 |
| | DRA:HLA-DQA1, HLA- | | | | | |
| | DRA:HLA-DQA1 | | | | | |
| 227404_s_at | EGR1 | 0.4851 | 0.5387 | 1.38 | 60.6 | 51-69.6 |
| 216401_x_at | LOC652745 | 0.4864 | 0.1728 | 1.09 | 53.4 | 43.9-62.8 |
| 216207_x_at | IGKV1D-13 | 0.5177 | 0.16 | 1.1 | 53.2 | 43.6-62.6 |
| 203477_at | COL15A1 | 0.6601 | 0.5385 | 1.22 | 60.6 | 51.1-69.7 |
| 211959_at | IGFBP5 | 0.7663 | 0.3526 | 1.25 | 57 | 47.4-66.2 |
| 201105_at | LGALS1 | 0.8555 | 0.4634 | 1.16 | 59.2 | 49.6-68.2 |
| 201438_at | COL6A3 | 0.8739 | 0.4434 | 1.2 | 58.8 | 49.2-67.8 |
| 212671_s_at | HLA-DQA1:HLA- | 0.8836 | 0.5398 | 1.28 | 60.6 | 51.1-69.7 |
| | DQA2, HLA- | | | | | |
| | DQA1:LOC731682:HLA- | | | | | |
| | DQA2 | | | | | |
| 227725_at | ST6GALNAC1 | 0.9452 | 0.2599 | 1.15 | 55.2 | 45.6-64.5 |
| 211990_at | HLA-DPA1, HLA-DPA1 | 0.9664 | 0.417 | 1.39 | 58.3 | 48.7-67.4 |
| 211991_s_at | HLA-DPA1, HLA-DPA1 | 0.9664 | 0.4166 | 1.39 | 58.3 | 48.7-67.4 |
| 217179_x_at | IGLV1-44 | 0.9777 | 0.2852 | 1.07 | 55.7 | 46.1-65 |
| 234764_x_at | IGLV1-44 | 0.9777 | 0.2847 | 1.07 | 55.7 | 46.1-64.9 |
| 224342_x_at | IGLV1-44 | 0.9777 | 0.2848 | 1.07 | 55.7 | 46.1-65 |
| 215176_x_at | NTN2L:IGKC:IGKV1- | 0.9858 | 0.4167 | 1.43 | 58.3 | 48.6-67.3 |
| | 5:GJB6:HLA-C | | | | | |
| 216576_x_at | NTN2L:IGKC:IGKV1- | 0.9858 | 0.4171 | 1.43 | 58.3 | 48.6-67.4 |
| | 5:GJB6:HLA-C | | | | | |
| 201645_at | TNC | 0.9905 | 0.3687 | 1.09 | 57.3 | 47.7-66.6 |
| 214414_x_at | HBA1:HBA2, HBA1:HBA2 | 0.9979 | 0.4707 | 1.21 | 59.3 | 49.7-68.3 |
| 204018_x_at | HBA1:HBA2, HBA1:HBA2 | 0.9979 | 0.4711 | 1.21 | 59.3 | 49.7-68.3 |
| 209458_x_at | HBA1:HBA2, HBA1:HBA2 | 0.9979 | 0.4727 | 1.21 | 59.3 | 49.7-68.4 |
| 211699_x_at | HBA1:HBA2, HBA1:HBA2 | 0.9979 | 0.4697 | 1.21 | 59.3 | 49.7-68.3 |
| 211745_x_at | HBA1:HBA2, HBA1:HBA2 | 0.9979 | 0.4708 | 1.21 | 59.3 | 49.8-68.3 |
| 217414_x_at | HBA1:HBA2, HBA1:HBA2 | 0.9979 | 0.469 | 1.21 | 59.3 | 49.7-68.4 |

TABLE 2

| Gene | | | | | Sens- | |
|---|---|---|---|---|---|---|
| Symbol | ValidPS_DOWN | Signif. FDR | D.val5 | FC | Spec | CI (95) |
| GCNT2 | 935239-HuGene_st:225205-HuGene_st:1026280- | 2.16E−27 | 3.9513 | 13.36 | 97.6 | 94.2-99.2 |
| | HuGene_st:668101-HuGene_st:1099985- | | | | | |
| | HuGene_st:698568-HuGene_st:134540- | | | | | |
| | HuGene_st:697147-HuGene_st:250092- | | | | | |
| | HuGene_st:611927-HuGene_st:972833- | | | | | |
| | HuGene_st:168891-HuGene_st:990860- | | | | | |
| | HuGene_st:109287-HuGene_st:322116- | | | | | |
| | HuGene_st:231019-HuGene_st:211020_at:959570- | | | | | |
| | HuGene_st:858764-HuGene_st:215593_at:820195- | | | | | |
| | HuGene_st:239606_at:41059-HuGene_st:669940- | | | | | |
| | HuGene_st:215595_x_at:230788_at | | | | | |
| GUCA2B | 276512-HuGene_st:1006871-HuGene_st:948364- | 7.04E−25 | 3.6268 | 51.78 | 96.5 | 92.4-98.6 |
| | HuGene_st:46575-HuGene_st:207502_at:436788- | | | | | |
| | HuGene_st:485636-HuGene_st:60132- | | | | | |
| | HuGene_st:608718-HuGene_st:779511- | | | | | |
| | HuGene_st:132431-HuGene_st:974232- | | | | | |
| | HuGene_st:425596-HuGene_st:308287- | | | | | |
| | HuGene_st:800088-HuGene_st:827119- | | | | | |
| | HuGene_st:233789-HuGene_st:623623-HuGene_st | | | | | |
| GUCA1B | 207003_at | 7.32E−25 | 3.5625 | 10.7 | 96.3 | 91.9-98.5 |
| CA4 | 636392-HuGene_st:206209_s_at:365396- | 1.73E−24 | 3.6291 | 10.41 | 96.5 | 92.4-98.7 |
| | HuGene_st:978203-HuGene_st:987169- | | | | | |
| | HuGene_st:638557-HuGene_st:206208_at:1033858- | | | | | |
| | HuGene_st:597808-HuGene_st:756864- | | | | | |
| | HuGene_st:326439-HuGene_st:356096- | | | | | |
| | HuGene_st:478435-HuGene_st:31642- | | | | | |
| | HuGene_st:682778-HuGene_st:1006162- | | | | | |
| | HuGene_st:673533-HuGene_st:491473- | | | | | |
| | HuGene_st:822299-HuGene_st:871453- | | | | | |
| | HuGene_st:209666-HuGene_st:465368- | | | | | |
| | HuGene_st:354896-HuGene_st | | | | | |
| SLC4A4 | 810103-HuGene_st:634869- | 2.23E−23 | 3.6301 | 3.58 | 96.5 | 92.4-98.6 |
| | HuGene_st:210739_x_at:211494_s_at:268096- | | | | | |
| | HuGene_st:70822-HuGene_st:940785- | | | | | |
| | HuGene_st:484105- | | | | | |
| | HuGene_st:210738_s_at:203908_at:99375- | | | | | |
| | HuGene_st:238833-HuGene_st:149464- | | | | | |
| | HuGene_st:874564-HuGene_st:161948- | | | | | |
| | HuGene_st:501640-HuGene_st:65687- | | | | | |
| | HuGene_st:886057-HuGene_st:495376- | | | | | |
| | HuGene_st:247215-HuGene_st:847550- | | | | | |
| | HuGene_st:190252-HuGene_st:244077- | | | | | |
| | HuGene_st:477911-HuGene_st:904959- | | | | | |
| | HuGene_st:218417-HuGene_st:1554027_a_at | | | | | |
| AQP8 | 486167-HuGene_st:126752-HuGene_st:93102- | 2.98E−22 | 3.3311 | 10.82 | 95.2 | 90.3-97.9 |
| | HuGene_st:1090469-HuGene_st:980308- | | | | | |
| | HuGene_st:107169-HuGene_st:829020- | | | | | |
| | HuGene_st:1053234-HuGene_st:954102- | | | | | |
| | HuGene_st:40331-HuGene_st:206784_at:990703- | | | | | |
| | HuGene_st:459706-HuGene_st:863327- | | | | | |
| | HuGene_st:965233-HuGene_st:459009- | | | | | |
| | HuGene_st:180228-HuGene_st:944315- | | | | | |
| | HuGene_st:814773-HuGene_st:810460- | | | | | |
| | HuGene_st:228837-HuGene_st | | | | | |
| CA1 | 632246-HuGene_st:205950_s_at:382371- | 3.15E−22 | 3.3666 | 38.27 | 95.4 | 90.6-98 |
| | HuGene_st:1004598-HuGene_st:405837- | | | | | |
| | HuGene_st:551543-HuGene_st:242482- | | | | | |
| | HuGene_st:267733-HuGene_st:1074039- | | | | | |
| | HuGene_st:107073-HuGene_st:485365- | | | | | |
| | HuGene_st:254908-HuGene_st:257502- | | | | | |
| | HuGene_st:333301-HuGene_st:180359- | | | | | |
| | HuGene_st:696950-HuGene_st:588557- | | | | | |
| | HuGene_st:381399-HuGene_st:495540- | | | | | |
| | HuGene_st:384192-HuGene_st:205949_at | | | | | |
| ABCG2 | 236197-HuGene_st:10623-HuGene_st:1005470- | 1.09E−21 | 3.2599 | 31.53 | 94.8 | 89.8-97.7 |
| | HuGene_st:140860-HuGene_st:492719- | | | | | |
| | HuGene_st:417387-HuGene_st:113831- | | | | | |
| | HuGene_st:507347-HuGene_st:944251- | | | | | |
| | HuGene_st:175319-HuGene_st:784920- | | | | | |
| | HuGene_st:709551-HuGene_st:136344- | | | | | |
| | HuGene_st:689536-HuGene_st:209735_at:167748- | | | | | |
| | HuGene_st:759590-HuGene_st:805222- | | | | | |
| | HuGene_st:945575-HuGene_st:294363- | | | | | |
| | HuGene_st:958524-HuGene_st:402982- | | | | | |
| | HuGene_st:301335-HuGene_st | | | | | |
| OSTbeta | 552746-HuGene_st:57742-HuGene_st:461622- | 6.23E−21 | 3.188 | 7.15 | 94.5 | 89.2-97.5 |
| | HuGene_st:965982-HuGene_st:413709- | | | | | |
| | HuGene_st:230830_at:232719-HuGene_st:165400- | | | | | |
| | HuGene_st:203597-HuGene_st:1025456- | | | | | |
| | HuGene_st:625341-HuGene_st:881755- | | | | | |
| | HuGene_st:490180-HuGene_st:113547- | | | | | |
| | HuGene_st:605512-HuGene_st:179662- | | | | | |
| | HuGene_st:280309-HuGene_st:647118-HuGene_st | | | | | |
| MGC13057 | 926449-HuGene_st:317301-HuGene_st:834797- | 6.94E−21 | 3.2029 | 3.18 | 94.5 | 89.3-97.5 |
| | HuGene_st:173388-HuGene_st:127937- | | | | | |
| | HuGene_st:182395-HuGene_st:800172- | | | | | |
| | HuGene_st:1041848-HuGene_st:93911- | | | | | |
| | HuGene_st:810617-HuGene_st:1035428- | | | | | |
| | HuGene_st:644866-HuGene_st:343402- | | | | | |
| | HuGene_st:223754_at:228195_at:509933-HuGene_st | | | | | |
| CLDN23 | 403960-HuGene_st:25144-HuGene_st:947653- | 4.27E−20 | 3.0777 | 3.47 | 93.8 | 88.2-97.1 |
| | HuGene_st:228704_s_at:228706_s_at:320375- | | | | | |
| | HuGene_st:441629:HuGene_st:367414- | | | | | |
| | HuGene_st:855269-HuGene_st:228707_at:788659- | | | | | |
| | HuGene_st:698816-HuGene_st:95789- | | | | | |
| | HuGene_st:270197-HuGene_st:472976- | | | | | |
| | HuGene_st:280539-HuGene_st:1056334- | | | | | |
| | HuGene_st:516288-HuGene_st:579963-HuGene_st | | | | | |
| PKIB | 866170-HuGene_st:1055812-HuGene_st:264946- | 5.94E−20 | 3.1232 | 3.28 | 94.1 | 88.7-97.3 |
| | HuGene_st:684057-HuGene_st:124791- | | | | | |
| | HuGene_st:134561-HuGene_st:1026756- | | | | | |
| | HuGene_st:468593-HuGene_st:1045852- | | | | | |
| | HuGene_st:939917-HuGene_st:110205- | | | | | |
| | HuGene_st:660721-HuGene_st:905229- | | | | | |
| | HuGene_st:223551_at:610426-HuGene_st | | | | | |
| SEMA6D | 855766-HuGene_st:233882_s_at:543564- | 6.07E−20 | 3.092 | 4.27 | 93.9 | 88.4-97.2 |
| | HuGene_st:277987-HuGene_st:1075165- | | | | | |
| | HuGene_st:220574_at:732853-HuGene_st:410940- | | | | | |
| | HuGene_st:384488-HuGene_st:1041056- | | | | | |
| | HuGene_st:233801_s_at:535378-HuGene_st:477671- | | | | | |
| | HuGene_st:626423-HuGene_st:101599- | | | | | |
| | HuGene_st:714098-HuGene_st:301505- | | | | | |
| | HuGene_st:53682-HuGene_st:177281- | | | | | |
| | HuGene_st:596415- | | | | | |
| | HuGene_st:244746_at:226492_at:699792- | | | | | |
| | HuGene_st:73748-HuGene_st:692437-HuGene_st | | | | | |
| CLCA4 | 220026_at:1000955-HuGene_st:215807- | 1.25E−19 | 3.1922 | 16.3 | 94.5 | 89.2-97.5 |
| | HuGene_st:108406-HuGene_st:538359- | | | | | |
| | HuGene_st:366726-HuGene_st:727431- | | | | | |
| | HuGene_st:240891-HuGene_st:485685- | | | | | |
| | HuGene_st:205213-HuGene_st:476179- | | | | | |
| | HuGene_st:283676-HuGene_st:933390- | | | | | |
| | HuGene_st:601908-HuGene_st:844633- | | | | | |
| | HuGene_st:846667-HuGene_st:99723- | | | | | |
| | HuGene_st:376041-HuGene_st:71188- | | | | | |
| | HuGene_st:261183-HuGene_st:498330- | | | | | |
| | HuGene_st:500064-HuGene_st:785031-HuGene_st | | | | | |
| TP53INP2 | 224836_at:294906-HuGene_st:857693- | 1.45E−19 | 3.0229 | 3.78 | 93.5 | 87.8-96.9 |
| | HuGene_st:852970-HuGene_st:594178- | | | | | |
| | HuGene_st:868173-HuGene_st:353925- | | | | | |
| | HuGene_st:212485-HuGene_st:804363- | | | | | |
| | HuGene_st:778869-HuGene_st:427072- | | | | | |
| | HuGene_st:451529-HuGene_st:808765- | | | | | |
| | HuGene_st:205314-HuGene_st:415164- | | | | | |
| | HuGene_st:1048358-HuGene_st | | | | | |
| MS4A12 | 220834_at:582400-HuGene_st:354845- | 2.13E−19 | 3.2962 | 7.15 | 95 | 90-97.8 |
| | HuGene_st:341876-HuGene_st:769180- | | | | | |
| | HuGene_st:740541-HuGene_st:436753- | | | | | |
| | HuGene_st:570027-HuGene_st:26519- | | | | | |
| | HuGene_st:956581-HuGene_st:335315- | | | | | |
| | HuGene_st:206721-HuGene_st:589444- | | | | | |
| | HuGene_st:16629-HuGene_st:113533- | | | | | |
| | HuGene_st:201614-HuGene_st:774878- | | | | | |
| | HuGene_st:786494-HuGene_st:493550- | | | | | |
| | HuGene_st:403871-HuGene_st:192686- | | | | | |
| | HuGene_st:846528-HuGene_st:249799-HuGene_st | | | | | |
| TRPM6 | 767074-HuGene_st:695352-HuGene_st:411125- | 2.25E−19 | 3.0106 | 7.5 | 93.4 | 87.7-96.8 |
| | HuGene_st:221102_s_at:234864_s_at:240389_at:358229- | | | | | |
| | HuGene_st:755964-HuGene_st:840301- | | | | | |
| | HuGene_st:959234-HuGene_st:782639- | | | | | |
| | HuGene_st:833079-HuGene_st:1066034- | | | | | |
| | HuGene_st:678013-HuGene_st:249083- | | | | | |
| | HuGene_st:143934-HuGene_st:159130- | | | | | |
| | HuGene_st:486486-HuGene_st:185057- | | | | | |
| | HuGene_st:878793-HuGene_st:133981- | | | | | |
| | HuGene_st:224412_s_at:202194-HuGene_st | | | | | |
| XLKD1 | 520080-HuGene_st:1091117-HuGene_st:943125- | 3.60E−19 | 2.94 | 7.44 | 92.9 | 87.1-96.6 |
| | HuGene_st:444068-HuGene_st:648558- | | | | | |
| | HuGene_st:346991-HuGene_st:1006205- | | | | | |
| | HuGene_st:373107-HuGene_st:682535- | | | | | |
| | HuGene_st:1083245-HuGene_st:863143- | | | | | |
| | HuGene_st:820120-HuGene_st:1044561- | | | | | |
| | HuGene_st:220037_s_at:541228-HuGene_st:220256- | | | | | |
| | HuGene_st:289122-HuGene_st:219059_s_at:246683- | | | | | |
| | HuGene_st:775976-HuGene_st:207399- | | | | | |
| | HuGene_st:1052557-HuGene_st:92121-HuGene_st | | | | | |
| ADH1B | 1078343-HuGene_st:512808-HuGene_st:614446- | 4.70E−19 | 3.0172 | 4.67 | 93.4 | 87.7-96.9 |
| | HuGene_st:910188-HuGene_st:422504- | | | | | |
| | HuGene_st:731361-HuGene_st:209612_s_at:258079- | | | | | |
| | HuGene_st:568239-HuGene_st:879930- | | | | | |
| | HuGene_st:420417-HuGene_st:1025048- | | | | | |
| | HuGene_st:908335-HuGene_st:654633- | | | | | |
| | HuGene_st:947292-HuGene_st:1087125- | | | | | |
| | HuGene_st:1004870-HuGene_st:209613_s_at:579636- | | | | | |
| | HuGene_st:681018-HuGene_st:822774-HuGene_st | | | | | |
| PRDX6 | 353187-HuGene_st:520885-HuGene_st:919334- | 5.56E−19 | 2.8414 | 2.76 | 92.2 | 86.1-96.1 |
| | HuGene_st:646105-HuGene_st:390352- | | | | | |
| | HuGene_st:200844_s_at:855125- | | | | | |
| | HuGene_st:200845_s_at:54231-HuGene_st:612931- | | | | | |
| | HuGene_st:837652-HuGene_st:538086- | | | | | |
| | HuGene_st:796167-HuGene_st:208992- | | | | | |
| | HuGene_st:39945-HuGene_st:489353- | | | | | |
| | HuGene_st:963499-HuGene_st:376244- | | | | | |
| | HuGene_st:297536-HuGene_st:495777- | | | | | |
| | HuGene_st:1005253-HuGene_st:833460-HuGene_st | | | | | |
| CA2 | 762141-HuGene_st:111686-HuGene_st:512916- | 5.87E−19 | 2.9625 | 9.22 | 93.1 | 87.2-96.7 |
| | HuGene_st:9132-HuGene_st:995083: | | | | | |
| | HuGene_st:209301_at:578483-HuGene_st:583428- | | | | | |
| | HuGene_st:961832-HuGene_st:11748- | | | | | |
| | HuGene_st:1056641-HuGene_st:545067- | | | | | |
| | HuGene_st:952781-HuGene_st:689246- | | | | | |
| | HuGene_st:246103-HuGene_st:803284- | | | | | |
| | HuGene_st:828842-HuGene_st:694989- | | | | | |
| | HuGene_st:808226-HuGene_st:199030-HuGene_st | | | | | |
| EDN3 | 314103-HuGene_st:217154_s_at:820613- | 6.42E−19 | 2.937 | 11.21 | 92.9 | 87-96.5 |
| | HuGene_st:1050996-HuGene_st:348157- | | | | | |
| | HuGene_st:338856-HuGene_st:594687- | | | | | |
| | HuGene_st:38595-HuGene_st:270770- | | | | | |
| | HuGene_st:817067-HuGene_st:766407- | | | | | |
| | HuGene_st:256284-HuGene_st:208399_s_at:200417- | | | | | |
| | HuGene_st:496738-HuGene_st:590141- | | | | | |
| | HuGene_st:137170-HuGene_st:499250- | | | | | |
| | HuGene_st:478524-HuGene_st:720694- | | | | | |
| | HuGene_st:543565-HuGene_st | | | | | |
| MIER3 | 884917-HuGene_st:740856-HuGene_st:107094- | 9.14E−19 | 2.9162 | 3.91 | 92.8 | 86.8-96.5 |
| | HuGene_st:408774-HuGene_st:461523- | | | | | |
| | HuGene_st:153511-HuGene_st:207042- | | | | | |
| | HuGene_st:737492-HuGene_st:43443- | | | | | |
| | HuGene_st:614740-HuGene_st:1559652_at:783045- | | | | | |
| | HuGene_st:887606-HuGene_st:996253- | | | | | |
| | HuGene_st:1041635-HuGene_st:142612- | | | | | |
| | HuGene_st:1553336_a_at:220555-HuGene_st:975347- | | | | | |
| | HuGene_st:470750-HuGene_st:1554450_s_at:804524- | | | | | |
| | HuGene_st:265536-HuGene_st:774142- | | | | | |
| | HuGene_st:231975_s_at:34726- | | | | | |
| | HuGene_st:1554449_at:228961_at | | | | | |
| ABCA8 | 57305-HuGene_st:111752-HuGene_st:61357- | 1.26E−18 | 2.9165 | 6.82 | 92.8 | 86.8-96.5 |
| | HuGene_st:123451-HuGene_st:740746- | | | | | |
| | HuGene_st:512280-HuGene_st:389185- | | | | | |
| | HuGene_st:427721-HuGene_st:346028- | | | | | |
| | HuGene_st:224566-HuGene_st:149653- | | | | | |
| | HuGene_st:680699-HuGene_st:76772- | | | | | |
| | HuGene_st:742091-HuGene_st:423333- | | | | | |
| | HuGene_st:559944-HuGene_st:341399- | | | | | |
| | HuGene_st:1565780_at:863400-HuGene_st:921748- | | | | | |
| | HuGene_st:623719-HuGene_st:204719_at:123143- | | | | | |
| | HuGene_st:1077391-HuGene_st | | | | | |
| METTL7A | 278779-HuGene_st:481332-HuGene_st:787584- | 1.35E−18 | 3.1231 | 1.93 | 94.1 | 88.6-97.3 |
| | HuGene_st:989793-HuGene_st:278513- | | | | | |
| | HuGene_st:424901-HuGene_st:551005- | | | | | |
| | HuGene_st:921169-HuGene_st:125176- | | | | | |
| | HuGene_st:277465-HuGene_st:560742- | | | | | |
| | HuGene_st:207761_s_at:367459-HuGene_st:224782- | | | | | |
| | HuGene_st:660194-HuGene_st:266654- | | | | | |
| | HuGene_st:1086300-HuGene_st:134633- | | | | | |
| | HuGene_st:211424_x_at:273973-HuGene_st:947408- | | | | | |
| | HuGene_st:209703_x_at:617184-HuGene_st:864197- | | | | | |
| | HuGene_st:10061-HuGene_st | | | | | |
| DPT | 876245-HuGene_st:1036210-HuGene_st:214758- | 1.53E−18 | 2.8977 | 9.07 | 92.6 | 86.7-96.4 |
| | HuGene_st:1005554-HuGene_st:178625- | | | | | |
| | HuGene_st:701967-HuGene_st:35076- | | | | | |
| | HuGene_st:33432-HuGene_st:897771- | | | | | |
| | HuGene_st:304494-HuGene_st:445757- | | | | | |
| | HuGene_st:491297-HuGene_st:646408- | | | | | |
| | HuGene_st:842542-HuGene_st:87795- | | | | | |
| | HuGene_st:213068_at:207977_s_at:553389- | | | | | |
| | HuGene_st:477075-HuGene_st:213071_at:1046282- | | | | | |
| | HuGene_st | | | | | |
| MGC4172 | 339712-HuGene_st:1089815-HuGene_st:265870- | 1.74E−18 | 2.9044 | 3.74 | 92.7 | 86.7-96.4 |
| | HuGene_st:895908-HuGene_st:226292- | | | | | |
| | HuGene_st:322910-HuGene_st:575887- | | | | | |
| | HuGene_st:416485-HuGene_st:1055581- | | | | | |
| | HuGene_st:1088692-HuGene_st:442194- | | | | | |
| | HuGene_st:218756_s_at:775684-HuGene_st:773137- | | | | | |
| | HuGene_st:373028-HuGene_st:512667- | | | | | |
| | HuGene_st:164027-HuGene_st:541185- | | | | | |
| | HuGene_st:593729-HuGene_st:506796-HuGene_st | | | | | |
| FNBP1 | 728822-HuGene_st:845505-HuGene_st:1054016- | 1.76E−18 | 3.0483 | 2.04 | 93.6 | 88-97 |
| | HuGene_st:383098-HuGene_st:230389_at:31865- | | | | | |
| | HuGene_st:981305-HuGene_st:213940_s_at:755179- | | | | | |
| | HuGene_st:839717- | | | | | |
| | HuGene_st:230086_at:239453_at:985213- | | | | | |
| | HuGene_st:125814:HuGene_st:451336- | | | | | |
| | HuGene_st:22306-HuGene_st:540957- | | | | | |
| | HuGene_st:277486-HuGene_st:314000- | | | | | |
| | HuGene_st:212288_at:662225-HuGene_st:524650- | | | | | |
| | HuGene_st:605840-HuGene_st:180978- | | | | | |
| | HuGene_st:604436-HuGene_st | | | | | |
| EDG2 | 816816-HuGene_st:309471-HuGene_st:77020- | 2.44E−18 | 2.8838 | 3.19 | 92.5 | 86.5-96.3 |
| | HuGene_st:289373-HuGene_st:10014- | | | | | |
| | HuGene_st:981213-HuGene_st:204038_s_at:1035537- | | | | | |
| | HuGene_st:75833-HuGene_st:781880- | | | | | |
| | HuGene_st:494373-HuGene_st:745317- | | | | | |
| | HuGene_st:497715-HuGene_st:693528- | | | | | |
| | HuGene_st:806817-HuGene_st:812294- | | | | | |
| | HuGene_st:204036_at:241773_at:176887- | | | | | |
| | HuGene_st:441141-HuGene_st:189399- | | | | | |
| | HuGene_st:723788-HuGene_st:204037_at:485543- | | | | | |
| | HuGene_st:1014086-HuGene_st:232716_at | | | | | |
| SFRP1 | 722739-HuGene_st:1095144-HuGene_st:1039611- | 2.77E−18 | 2.8631 | 15.53 | 92.4 | 86.3-96.2 |
| | HuGene_st:836419-HuGene_st:34446- | | | | | |
| | HuGene_st:50826-HuGene_st:483033- | | | | | |
| | HuGene_st:202036_s_at:988375-HuGene_st:774922- | | | | | |
| | HuGene_st:228413_s_at:665988-HuGene_st:302344- | | | | | |
| | HuGene_st:202035_s_at:412438-HuGene_st:995086- | | | | | |
| | HuGene_st:202037_s_at:181713-HuGene_st:527962- | | | | | |
| | HuGene_st:880207-HuGene_st:100260-HuGene_st | | | | | |
| SLC26A3 | 206143_at:172542-HuGene_st:768419- | 4.18E−18 | 2.9214 | 21.4 | 92.8 | 86.8-96.5 |
| | HuGene_st:820082-HuGene_st:263997- | | | | | |
| | HuGene_st:681117-HuGene_st:581666- | | | | | |
| | HuGene_st:180553-HuGene_st:356894- | | | | | |
| | HuGene_st:240715-HuGene_st:718555- | | | | | |
| | HuGene_st:517735-HuGene_st:339388- | | | | | |
| | HuGene_st:989337-HuGene_st:1511- | | | | | |
| | HuGene_st:228629-HuGene_st:354149- | | | | | |
| | HuGene_st:603695-HuGene_st:791438- | | | | | |
| | HuGene_st:154730-HuGene_st:905529- | | | | | |
| | HuGene_st:248690-HuGene_st:431441- | | | | | |
| | HuGene_st:215657_at | | | | | |
| ANGPTL1 | 572942-HuGene_st:891312-HuGene_st:953040- | 1.42E−17 | 2.7929 | 4.64 | 91.9 | 85.6-95.9 |
| | HuGene_st:232844-HuGene_st:145730- | | | | | |
| | HuGene_st:142205-HuGene_st:227771- | | | | | |
| | HuGene_st:80584-HuGene_st:982090- | | | | | |
| | HuGene_st:999640-HuGene_st:672931- | | | | | |
| | HuGene_st:148578-HuGene_st:224339_s_at:1046706- | | | | | |
| | HuGene_st:239183_at:155660-HuGene_st:284674- | | | | | |
| | HuGene_st:231773_at:818064-HuGene_st:978991- | | | | | |
| | HuGene_st:728775-HuGene_st | | | | | |
| UGP2 | 795967-HuGene_st:561220-HuGene_st:754258- | 1.64E−17 | 2.7904 | 2.41 | 91.9 | 85.6-95.9 |
| | HuGene_st:846757-HuGene_st:868436- | | | | | |
| | HuGene_st:321605-HuGene_st:299097- | | | | | |
| | HuGene_st:7536-HuGene_st:216268- | | | | | |
| | HuGene_st:925542-HuGene_st:967807- | | | | | |
| | HuGene_st:205480_s_at:639621-HuGene_st:177627- | | | | | |
| | HuGene_st:894271-HuGene_st:617530- | | | | | |
| | HuGene_st:231698_at:971369-HuGene_st:132663- | | | | | |
| | HuGene_st:215198-HuGene_st:647604- | | | | | |
| | HuGene_st:537579-HuGene_st:232180_at:1056881- | | | | | |
| | HuGene_st | | | | | |
| SRPX | 195599-HuGene_st:541428-HuGene_st:143706- | 1.79E−17 | 2.7749 | 3.91 | 91.7 | 85.5-95.8 |
| | HuGene_st:728558-HuGene_st:190938- | | | | | |
| | HuGene_st:455214-HuGene_st:522280- | | | | | |
| | HuGene_st:966576-HuGene_st:650551- | | | | | |
| | HuGene_st:810331-HuGene_st:396246- | | | | | |
| | HuGene_st:133110-HuGene_st:232310- | | | | | |
| | HuGene_st:475511-HuGene_st:225561- | | | | | |
| | HuGene_st:351281-HuGene_st:204955_at | | | | | |
| OGN | 955964-HuGene_st:775073-HuGene_st:678354- | 2.25E−17 | 2.7078 | 13.67 | 91.2 | 84.7-95.4 |
| | HuGene_st:238500-HuGene_st:1019070- | | | | | |
| | HuGene_st:614445-HuGene_st:23784- | | | | | |
| | HuGene_st:658925-HuGene_st:757128- | | | | | |
| | HuGene_st:632689-HuGene_st:989474- | | | | | |
| | HuGene_st:233177-HuGene_st:650498- | | | | | |
| | HuGene_st:950000-HuGene_st:751146- | | | | | |
| | HuGene_st:67232-HuGene_st:997991- | | | | | |
| | HuGene_st:218730_s_at:222722_at:821488- | | | | | |
| | HuGene_st | | | | | |
| MYLK | 527465-HuGene_st:617817-HuGene_st:716971- | 2.33E−17 | 2.7729 | 3.99 | 91.7 | 85.4-95.8 |
| | HuGene_st:942711-HuGene_st:230460_at:161532- | | | | | |
| | HuGene_st:767103-HuGene_st:1041083- | | | | | |
| | HuGene_st:202167-HuGene_st:738247- | | | | | |
| | HuGene_st:505966-HuGene_st:62701- | | | | | |
| | HuGene_st:776564-HuGene_st:679137- | | | | | |
| | HuGene_st:551306- | | | | | |
| | HuGene_st:202555_s_at:224823_at:986313- | | | | | |
| | HuGene_st:324943-HuGene_st:218172- | | | | | |
| | HuGene_st:351524-HuGene_st:1568770_at:280094- | | | | | |
| | HuGene_st:1017324-HuGene_st:1563466_at | | | | | |
| LIFR | 275506-HuGene_st:323055-HuGene_st:444251- | 2.45E−17 | 2.7826 | 6.36 | 91.8 | 85.6-95.8 |
| | HuGene_st:1056178-HuGene_st:398104- | | | | | |
| | HuGene_st:917434-HuGene_st:1044918- | | | | | |
| | HuGene_st:167500-HuGene_st:423760- | | | | | |
| | HuGene_st:837336-HuGene_st:321505- | | | | | |
| | HuGene_st:918321-HuGene_st:252278- | | | | | |
| | HuGene_st:884504-HuGene_st:124845- | | | | | |
| | HuGene_st:499777-HuGene_st:969722- | | | | | |
| | HuGene_st:709439-HuGene_st:611505- | | | | | |
| | HuGene_st:227771_at:287217- | | | | | |
| | HuGene_st:205876_at:225571_at:229185_at:233367_at: | | | | | |
| | 1093011-HuGene_st | | | | | |
| ZG16 | 287680-HuGene_st:1704-HuGene_st:61566- | 2.51E−17 | 2.8097 | 8.14 | 92 | 85.8-96 |
| | HuGene_st:964936-HuGene_st:9737- | | | | | |
| | HuGene_st:620497-HuGene_st:785765- | | | | | |
| | HuGene_st:214142_at:323025-HuGene_st:1093257- | | | | | |
| | HuGene_st:398415-HuGene_st:942991- | | | | | |
| | HuGene_st:871899-HuGene_st:548837- | | | | | |
| | HuGene_st:605328-HuGene_st:231918- | | | | | |
| | HuGene_st:461816-HuGene_st:604283- | | | | | |
| | HuGene_st:971003-HuGene_st:750108- | | | | | |
| | HuGene_st:927723-HuGene_st:782940- | | | | | |
| | HuGene_st:98669-HuGene_st | | | | | |
| EPB41L3 | 275431-HuGene_st:540074-HuGene_st:731277- | 2.62E−17 | 2.8312 | 2.51 | 92.2 | 86-96.1 |
| | HuGene_st:211776_s_at:218157-HuGene_st:328922- | | | | | |
| | HuGene_st:384314-HuGene_st:272486- | | | | | |
| | HuGene_st:196590-HuGene_st:294230- | | | | | |
| | HuGene_st:206710_s_at:747631-HuGene_st:52743- | | | | | |
| | HuGene_st:1089957-HuGene_st:423337- | | | | | |
| | HuGene_st:915755-HuGene_st:623682- | | | | | |
| | HuGene_st:130815-HuGene_st:497574- | | | | | |
| | HuGene_st:212681_at:77106-HuGene_st:874781- | | | | | |
| | HuGene_st | | | | | |
| SCNN1B | 773390-HuGene_st:467376-HuGene_st:411583- | 3.20E−17 | 2.7219 | 16.15 | 91.3 | 84.9-95.5 |
| | HuGene_st:433511-HuGene_st:142579- | | | | | |
| | HuGene_st:99655-HuGene_st:278054- | | | | | |
| | HuGene_st:371417-HuGene_st:99189- | | | | | |
| | HuGene_st:491965-HuGene_st:490957- | | | | | |
| | HuGene_st:916581-HuGene_st:285916- | | | | | |
| | HuGene_st:359156-HuGene_st:977951- | | | | | |
| | HuGene_st:291765-HuGene_st:767266- | | | | | |
| | HuGene_st:206091-HuGene_st:348267- | | | | | |
| | HuGene_st:205464_at:838576-HuGene_st | | | | | |
| HHLA2 | 371335-HuGene_st:978721-HuGene_st:1065567- | 3.47E−17 | 2.6741 | 10.02 | 90.9 | 84.4-95.2 |
| | HuGene_st:282548-HuGene_st:240410- | | | | | |
| | HuGene_st:170899-HuGene_st:947848- | | | | | |
| | HuGene_st:438234-HuGene_st:220812_s_at:927495- | | | | | |
| | HuGene_st:351364-HuGene_st:234673_at:993142- | | | | | |
| | HuGene_st:1009637-HuGene_st:335000- | | | | | |
| | HuGene_st:285313-HuGene_st:533646- | | | | | |
| | HuGene_st:234624_at:458597-HuGene_st:104838- | | | | | |
| | HuGene_st:26687-HuGene_st:258409- | | | | | |
| | HuGene_st:493304-HuGene_st:378019- | | | | | |
| | HuGene_st:576796-HuGene_st | | | | | |
| HPGD | 291863-HuGene_st:375608-HuGene_st:793406- | 3.65E−17 | 2.8433 | 3.21 | 92.2 | 86.1-96.1 |
| | HuGene_st:436293-HuGene_st:75568- | | | | | |
| | HuGene_st:211549_s_at:684728-HuGene_st:674596- | | | | | |
| | HuGene_st:527856-HuGene_st:329920- | | | | | |
| | HuGene_st:748432-HuGene_st:259392- | | | | | |
| | HuGene_st:769902-HuGene_st:620673- | | | | | |
| | HuGene_st:450707-HuGene_st:203913_s_at:304752- | | | | | |
| | HuGene_st:447604-HuGene_st:170968- | | | | | |
| | HuGene_st:852359-HuGene_st:836377- | | | | | |
| | HuGene_st:242733_at:243846-HuGene_st:136281- | | | | | |
| | HuGene_st:203914_x_at:211548_s_at:288252- | | | | | |
| | HuGene_st | | | | | |
| CEACAM7 | 206199_at:105665-HuGene_st:481561- | 3.96E−17 | 2.785 | 10.4 | 91.8 | 85.5-95.8 |
| | HuGene_st:211848_s_at:574369-HuGene_st:104615- | | | | | |
| | HuGene_st:242258:HuGene_st:740634- | | | | | |
| | HuGene_st:439249-HuGene_st:15044- | | | | | |
| | HuGene_st:424954-HuGene_st:1002484- | | | | | |
| | HuGene_st:206198_s_at:1067871-HuGene_st:751891- | | | | | |
| | HuGene_st:36594-HuGene_st:298155- | | | | | |
| | HuGene_st:1024423-HuGene_st:384378- | | | | | |
| | HuGene_st:1093665-HuGene_st:985998- | | | | | |
| | HuGene_st:325578-HuGene_st:596079- | | | | | |
| | HuGene_st:89770-HuGene_st:864758- | | | | | |
| | HuGene_st:205004-HuGene_st:374052- | | | | | |
| | HuGene_st:133822-HuGene_st:1093942- | | | | | |
| | HuGene_st:780375-HuGene_st:338636-HuGene_st | | | | | |
| RELL1 | 753672-HuGene_st:871189-HuGene_st:202462- | 4.10E−17 | 2.6983 | 2.5 | 91.1 | 84.6-95.4 |
| | HuGene_st:615056-HuGene_st:639180- | | | | | |
| | HuGene_st:4103-HuGene_st:778017- | | | | | |
| | HuGene_st:236549-HuGene_st:86799- | | | | | |
| | HuGene_st:908328-HuGene_st:813998- | | | | | |
| | HuGene_st:859771-HuGene_st:254041- | | | | | |
| | HuGene_st:787272-HuGene_st:916269- | | | | | |
| | HuGene_st:885786-HuGene_st:1554714_at:645386- | | | | | |
| | HuGene_st:777480-HuGene_st:927821- | | | | | |
| | HuGene_st:226430_at | | | | | |
| C7 | 893214-HuGene_st:54339-HuGene_st:501090- | 4.40E−17 | 2.7158 | 6.67 | 91.3 | 84.9-95.5 |
| | HuGene_st:659367-HuGene_st:536407- | | | | | |
| | HuGene_st:598145-HuGene_st:444111- | | | | | |
| | HuGene_st:643669-HuGene_st:191981- | | | | | |
| | HuGene_st:832001-HuGene_st:313065- | | | | | |
| | HuGene_st:612836-HuGene_st:673870- | | | | | |
| | HuGene_st:1005162-HuGene_st:206652- | | | | | |
| | HuGene_st:1021871-HuGene_st:489813- | | | | | |
| | HuGene_st:350161-HuGene_st:766550- | | | | | |
| | HuGene_st:2377-HuGene_st:202992_at:578639- | | | | | |
| | HuGene_st | | | | | |
| PLCE1 | 1562826_at:1563103_at:699801-HuGene_st:353862- | 6.91E−17 | 2.7528 | 2.43 | 91.6 | 85.2-95.7 |
| | HuGene_st:187026-HuGene_st:475246- | | | | | |
| | HuGene_st:819995-HuGene_st:477396- | | | | | |
| | HuGene_st:920273-HuGene_st:1566739_at:158053- | | | | | |
| | HuGene_st:554291-HuGene_st:854670- | | | | | |
| | HuGene_st:950080-HuGene_st:1064466- | | | | | |
| | HuGene_st:1017434-HuGene_st:987146- | | | | | |
| | HuGene_st:137607-HuGene_st:833052- | | | | | |
| | HuGene_st:205112_at:987189-HuGene_st:1092203- | | | | | |
| | HuGene_st:449587- | | | | | |
| | HuGene_st:205111_s_at:1566740_at:928426- | | | | | |
| | HuGene_st:649071-HuGene_st | | | | | |
| SCARA5 | 734536-HuGene_st:1080104-HuGene_st:796100- | 1.25E−16 | 2.6912 | 2.86 | 91.1 | 84.5-95.3 |
| | HuGene_st:258146-HuGene_st:670462- | | | | | |
| | HuGene_st:362431-HuGene_st:1019057- | | | | | |
| | HuGene_st:287988-HuGene_st:235849_at:222306- | | | | | |
| | HuGene_st:495319-HuGene_st:84286- | | | | | |
| | HuGene_st:47679-HuGene_st:855551- | | | | | |
| | HuGene_st:229839_at:999202-HuGene_st:982358- | | | | | |
| | HuGene_st:86142-HuGene_st:568080- | | | | | |
| | HuGene_st:109115-HuGene_st:590235- | | | | | |
| | HuGene_st:1554705_at:800351-HuGene_st | | | | | |
| CHRDL1 | 335657-HuGene_st:1058673-HuGene_st:166016- | 1.49E−16 | 2.6092 | 13.88 | 90.4 | 83.7-94.9 |
| | HuGene_st:825273-HuGene_st:35456- | | | | | |
| | HuGene_st:91115-HuGene_st:303433- | | | | | |
| | HuGene_st:209044-HuGene_st:1081642- | | | | | |
| | HuGene_st:135625-HuGene_st:284037- | | | | | |
| | HuGene_st:452295-HuGene_st:313790- | | | | | |
| | HuGene_st:912814-HuGene_st:712513- | | | | | |
| | HuGene_st:1078238-HuGene_st:721877- | | | | | |
| | HuGene_st:209763_at:657006-HuGene_st | | | | | |
| NR3C2 | 748063-HuGene_st:177629-HuGene_st:964944- | 2.08E−16 | 2.5811 | 3.19 | 90.2 | 83.3-94.7 |
| | HuGene_st:346404-HuGene_st:120283- | | | | | |
| | HuGene_st:646104-HuGene_st:731385- | | | | | |
| | HuGene_st:493776-HuGene_st:440054- | | | | | |
| | HuGene_st:286582-HuGene_st:1097927- | | | | | |
| | HuGene_st:804888-HuGene_st:234142_at:49341- | | | | | |
| | HuGene_st:370558-HuGene_st:22007- | | | | | |
| | HuGene_st:981978-HuGene_st:100953- | | | | | |
| | HuGene_st:239673_at:149241-HuGene_st:940356- | | | | | |
| | HuGene_st:628366- | | | | | |
| | HuGene_st:205259_at:1564236_at:328672-HuGene_st | | | | | |
| TSPAN7 | 703402-HuGene_st:745610-HuGene_st:1018812- | 2.13E−16 | 2.651 | 3.68 | 90.7 | 84.2-95.1 |
| | HuGene_st:742943-HuGene_st:168712- | | | | | |
| | HuGene_st:234246_at:902239-HuGene_st:285968- | | | | | |
| | HuGene_st:883914-HuGene_st:113610- | | | | | |
| | HuGene_st:236336-HuGene_st:511310- | | | | | |
| | HuGene_st:40850-HuGene_st:346422- | | | | | |
| | HuGene_st:189042-HuGene_st:745521- | | | | | |
| | HuGene_st:996022-HuGene_st:202242_at:195303- | | | | | |
| | HuGene_st:70610-HuGene_st:31995- | | | | | |
| | HuGene_st:234245_at | | | | | |
| STMN2 | 459270-HuGene_st:611227-HuGene_st:295241- | 2.25E−16 | 2.6147 | 6.13 | 90.4 | 83.8-94.9 |
| | HuGene_st:935618-HuGene_st:161827- | | | | | |
| | HuGene_st:17520-HuGene_st:337557- | | | | | |
| | HuGene_st:16478-HuGene_st:1015231- | | | | | |
| | HuGene_st:790658-HuGene_st:892767- | | | | | |
| | HuGene_st:343589-HuGene_st:1047491- | | | | | |
| | HuGene_st:454759-HuGene_st:9820- | | | | | |
| | HuGene_st:203001_s_at:472072- | | | | | |
| | HuGene_st:203000_at:691598-HuGene_st:1064573- | | | | | |
| | HuGene_st:811444-HuGene_st:485134-HuGene_st | | | | | |
| FAM107A | 315531-HuGene_st:294482-HuGene_st:764207- | 2.52E−16 | 2.6168 | 2.92 | 90.5 | 83.8-94.9 |
| | HuGene_st:809908-HuGene_st:209074_s_at:341591- | | | | | |
| | HuGene_st:948705-HuGene_st:207547_s_at:415418- | | | | | |
| | HuGene_st:215297-HuGene_st:332641-HuGene_st | | | | | |
| ANK2 | 314086-HuGene_st:282874-HuGene_st:382431- | 2.59E−16 | 2.6501 | 6.76 | 90.7 | 84.1-95.1 |
| | HuGene_st:229308-HuGene_st:779600- | | | | | |
| | HuGene_st:297624-HuGene_st:385943- | | | | | |
| | HuGene_st:730140-HuGene_st:442277- | | | | | |
| | HuGene_st:699309-HuGene_st:182816- | | | | | |
| | HuGene_st:202921_s_at:799860-HuGene_st:868462- | | | | | |
| | HuGene_st:634421-HuGene_st:571536- | | | | | |
| | HuGene_st:1050903-HuGene_st:649509- | | | | | |
| | HuGene_st:239935-HuGene_st:202920_at | | | | | |
| CLIC5 | 217628_at:219866_at:116794-HuGene_st:114619- | 3.68E−16 | 2.6963 | 2.37 | 91.1 | 84.6-95.4 |
| | HuGene_st:895974-HuGene_st:749788- | | | | | |
| | HuGene_st:485909-HuGene_st:326032- | | | | | |
| | HuGene_st:1083118-HuGene_st:213317_at:507492- | | | | | |
| | HuGene_st:480370-HuGene_st:160114- | | | | | |
| | HuGene_st:637800-HuGene_st:33914- | | | | | |
| | HuGene_st:1044626-HuGene_st:243917_at:664038- | | | | | |
| | HuGene_st:867787-HuGene_st:261656- | | | | | |
| | HuGene_st:399156-HuGene_st:558641-HuGene_st | | | | | |
| CHGA | 152790-HuGene_st:354161-HuGene_st:365802- | 5.54E−16 | 2.6097 | 7.21 | 90.4 | 83.6-94.9 |
| | HuGene_st:813261-HuGene_st:40004- | | | | | |
| | HuGene_st:504502-HuGene_st:624128- | | | | | |
| | HuGene_st:719186-HuGene_st:916335- | | | | | |
| | HuGene_st:921714-HuGene_st:489360- | | | | | |
| | HuGene_st:849455-HuGene_st:231475- | | | | | |
| | HuGene_st:294992-HuGene_st:204697_s_at:659997- | | | | | |
| | HuGene_st:521835-HuGene_st:659283- | | | | | |
| | HuGene_st:890388-HuGene_st:786316-HuGene_st | | | | | |
| KIAA0828 | 613787-HuGene_st:478932-HuGene_st:67325- | 5.58E−16 | 2.5514 | 4.09 | 89.9 | 83-94.5 |
| | HuGene_st:668897-HuGene_st:160683- | | | | | |
| | HuGene_st:1031978-HuGene_st:1018444- | | | | | |
| | HuGene_st:237998-HuGene_st:892138- | | | | | |
| | HuGene_st:262221-HuGene_st:864937- | | | | | |
| | HuGene_st:917541-HuGene_st:593865- | | | | | |
| | HuGene_st:55857-HuGene_st:215672_s_at:509888- | | | | | |
| | HuGene_st:302988-HuGene_st:668914- | | | | | |
| | HuGene_st:507489-HuGene_st:212814_at | | | | | |
| EDIL3 | 436107-HuGene_st:1558643_s_at:214345_at:668780- | 6.21E−16 | 2.6259 | 2.96 | 90.5 | 83.8-95 |
| | HuGene_st:545521-HuGene_st:641822- | | | | | |
| | HuGene_st:207379_at:411378-HuGene_st:485618- | | | | | |
| | HuGene_st:576523-HuGene_st:843058- | | | | | |
| | HuGene_st:910789-HuGene_st:165720- | | | | | |
| | HuGene_st:79965-HuGene_st:533665- | | | | | |
| | HuGene_st:237605-HuGene_st:593615- | | | | | |
| | HuGene_st:396108-HuGene_st:701416- | | | | | |
| | HuGene_st:325630-HuGene_st:912197- | | | | | |
| | HuGene_st:224797-HuGene_st:724765- | | | | | |
| | HuGene_st:167229- | | | | | |
| | HuGene_st:233668_at:225275_at:16759- | | | | | |
| | HuGene_st:233875_at | | | | | |
| DSCR1 | 535011-HuGene_st:789105- | 6.21E−16 | 2.6122 | 2.14 | 90.4 | 83.7-94.9 |
| | HuGene_st:215253_s_at:838760-HuGene_st:617570- | | | | | |
| | HuGene_st:419279-HuGene_st:1015938- | | | | | |
| | HuGene_st:208370_s_at:131478-HuGene_st:228884- | | | | | |
| | HuGene_st:685397-HuGene_st:68677- | | | | | |
| | HuGene_st:215254_at:656173-HuGene_st:971107- | | | | | |
| | HuGene_st:1039926-HuGene_st:206974- | | | | | |
| | HuGene_st:232227-HuGene_st:546248- | | | | | |
| | HuGene_st:175597-HuGene_st:1558181_at | | | | | |
| PPAP2A | 282276-HuGene_st:1062765-HuGene_st:952258- | 6.43E−16 | 2.596 | 2.4 | 90.3 | 83.6-94.8 |
| | HuGene_st:214647-HuGene_st:153982- | | | | | |
| | HuGene_st:678140-HuGene_st:209147_s_at:1083041- | | | | | |
| | HuGene_st:459000-HuGene_st:505597- | | | | | |
| | HuGene_st:1035745-HuGene_st:576550- | | | | | |
| | HuGene_st:243715_at:180473-HuGene_st:527974- | | | | | |
| | HuGene_st:821572-HuGene_st:260389- | | | | | |
| | HuGene_st:1042377-HuGene_st:138155- | | | | | |
| | HuGene_st:210946_at:243442_x_at | | | | | |
| PDCD4 | 681770-HuGene_st:1029160-HuGene_st:594740- | 8.67E−16 | 2.4578 | 2.6 | 89 | 82-93.9 |
| | HuGene_st:247013-HuGene_st:845755- | | | | | |
| | HuGene_st:813933-HuGene_st:367555- | | | | | |
| | HuGene_st:456148-HuGene_st:295280- | | | | | |
| | HuGene_st:144200-HuGene_st:620666- | | | | | |
| | HuGene_st:212593_s_at:590149-HuGene_st:87471- | | | | | |
| | HuGene_st:189624-HuGene_st:202730_s_at:44562- | | | | | |
| | HuGene_st:641673-HuGene_st:577621- | | | | | |
| | HuGene_st:102842-HuGene_st:202731_at:776615- | | | | | |
| | HuGene_st:615983-HuGene_st:212594_at:1557166_at | | | | | |
| DHRS9 | 413956-HuGene_st:371161- | 1.09E−15 | 2.7371 | 1.51 | 91.4 | 85.1-95.6 |
| | HuGene_st:224009_x_at:61832-HuGene_st:874913- | | | | | |
| | HuGene_st:321055-HuGene_st:223952_x_at:916038- | | | | | |
| | HuGene_st:313731-HuGene_st:629747- | | | | | |
| | HuGene_st:836048-HuGene_st:369374- | | | | | |
| | HuGene_st:157673-HuGene_st:194386- | | | | | |
| | HuGene_st:1089433-HuGene_st:352973- | | | | | |
| | HuGene_st:637905-HuGene_st:1010893- | | | | | |
| | HuGene_st:219799_s_at:361862-HuGene_st:800910- | | | | | |
| | HuGene_st:1012296-HuGene_st | | | | | |
| CDKN2B | 252481-HuGene_st:1000015-HuGene_st:172198- | 1.34E−15 | 2.5433 | 10.09 | 89.8 | 82.9-94.5 |
| | HuGene_st:967979-HuGene_st:675431- | | | | | |
| | HuGene_st:605224-HuGene_st:277126- | | | | | |
| | HuGene_st:846115-HuGene_st:452433- | | | | | |
| | HuGene_st:332731-HuGene_st:563208- | | | | | |
| | HuGene_st:527577-HuGene_st:207530_s_at:891201- | | | | | |
| | HuGene_st:512260-HuGene_st:236313_at:26244- | | | | | |
| | HuGene_st:61586-HuGene_st | | | | | |
| P2RY1 | 628249-HuGene_st:113454-HuGene_st:627857- | 1.41E−15 | 2.5341 | 3.6 | 89.7 | 82.8-94.4 |
| | HuGene_st:461281-HuGene_st:207455_at:259065- | | | | | |
| | HuGene_st:797734-HuGene_st:135788- | | | | | |
| | HuGene_st:42916-HuGene_st:315405- | | | | | |
| | HuGene_st:340050-HuGene_st:173225- | | | | | |
| | HuGene_st:919818-HuGene_st:591228- | | | | | |
| | HuGene_st:899117-HuGene_st:785070- | | | | | |
| | HuGene_st:286200-HuGene_st:231925_at | | | | | |
| LOC646627 | 238143_at | 1.58E−15 | 1.9131 | 5.02 | 83.1 | 74.9-89.4 |
| PPAP2B | 484735-HuGene_st:860271-HuGene_st:501067- | 2.44E−15 | 2.5819 | 1.81 | 90.2 | 83.4-94.7 |
| | HuGene_st:180731-HuGene_st:919245- | | | | | |
| | HuGene_st:209355_s_at:322730-HuGene_st:830995- | | | | | |
| | HuGene_st:212226_s_at:353268-HuGene_st:989778- | | | | | |
| | HuGene_st:279549-HuGene_st:583834- | | | | | |
| | HuGene_st:1035367-HuGene_st:155537- | | | | | |
| | HuGene_st:782552-HuGene_st:867734- | | | | | |
| | HuGene_st:267847-HuGene_st:212230_at:619833- | | | | | |
| | HuGene_st:232324_x_at | | | | | |
| PYY | 656845-HuGene_st:816022-HuGene_st:633572- | 3.50E−15 | 2.4793 | 11.71 | 89.2 | 82.2-94 |
| | HuGene_st:20355-HuGene_st:240779- | | | | | |
| | HuGene_st:638358-HuGene_st:879780- | | | | | |
| | HuGene_st:207080_s_at:211253_x_at:368591- | | | | | |
| | HuGene_st | | | | | |
| CLEC3B | 794375-HuGene_st:894763-HuGene_st:152328- | 4.65E−15 | 2.4437 | 4.46 | 88.9 | 81.7-93.8 |
| | HuGene_st:669650-HuGene_st:1090081- | | | | | |
| | HuGene_st:251162-HuGene_st:264101- | | | | | |
| | HuGene_st:367416-HuGene_st:984078- | | | | | |
| | HuGene_st:512272-HuGene_st:166348- | | | | | |
| | HuGene_st:181414-HuGene_st:995862- | | | | | |
| | HuGene_st:603678-HuGene_st:205200_at:56968- | | | | | |
| | HuGene_st:542483-HuGene_st | | | | | |
| NDE1 | 228133_s_at:218414_s_at:555730-HuGene_st:620739- | 4.84E−15 | 2.5125 | 1.99 | 89.5 | 82.6-94.3 |
| | HuGene_st:111363-HuGene_st | | | | | |
| CCL8 | 402294-HuGene_st:1098932-HuGene_st:930533- | 5.75E−15 | 2.4512 | 7.51 | 89 | 81.9-93.9 |
| | HuGene_st:524795-HuGene_st:574692- | | | | | |
| | HuGene_st:314202-HuGene_st:719831- | | | | | |
| | HuGene_st:410906-HuGene_st:643479- | | | | | |
| | HuGene_st:911966-HuGene_st:31200- | | | | | |
| | HuGene_st:547634-HuGene_st:39456- | | | | | |
| | HuGene_st:527508-HuGene_st:1057270- | | | | | |
| | HuGene_st:1062460-HuGene_st:214038_at | | | | | |
| PLAC8 | 219014_at:739479-HuGene_st:354260- | 5.76E−15 | 2.4228 | 3.87 | 88.7 | 81.5-93.7 |
| | HuGene_st:994047-HuGene_st:456534- | | | | | |
| | HuGene_st:236727_at:330382-HuGene_st:118286- | | | | | |
| | HuGene_st:940467-HuGene_st:493579- | | | | | |
| | HuGene_st:37786-HuGene_st:776403- | | | | | |
| | HuGene_st:295172-HuGene_st:271859- | | | | | |
| | HuGene_st:438305-HuGene_st:770531- | | | | | |
| | HuGene_st:982881-HuGene_st:543535- | | | | | |
| | HuGene_st:554805-HuGene_st:667817- | | | | | |
| | HuGene_st:519806-HuGene_st:1065533- | | | | | |
| | HuGene_st:345940-HuGene_st | | | | | |
| MUCDHL | 690807-HuGene_st:11068-HuGene_st:955242- | 5.81E−15 | 2.4358 | 3.06 | 88.8 | 81.7-93.8 |
| | HuGene_st:969280-HuGene_st:220075_s_at:625411- | | | | | |
| | HuGene_st:789310-HuGene_st:444568- | | | | | |
| | HuGene_st:589749-HuGene_st:265998- | | | | | |
| | HuGene_st:275576-HuGene_st:219796_s_at:625643- | | | | | |
| | HuGene_st:452184-HuGene_st:692224- | | | | | |
| | HuGene_st:1009431-HuGene_st:311533- | | | | | |
| | HuGene_st:289109-HuGene_st:101091- | | | | | |
| | HuGene_st:220074_at:307121-HuGene_st | | | | | |
| SULT1A1 | 322199-HuGene_st:1021081-HuGene_st:372215- | 7.17E−15 | 2.422 | 3.37 | 88.7 | 81.6-93.7 |
| | HuGene_st:689177-HuGene_st:215299_x_at:369102- | | | | | |
| | HuGene_st:203615_x_at:761577-HuGene_st:744847- | | | | | |
| | HuGene_st:691837-HuGene_st:2491- | | | | | |
| | HuGene_st:742631-HuGene_st:1037597- | | | | | |
| | HuGene_st:805936-HuGene_st | | | | | |
| SDCBP2 | 233565_s_at:263537-HuGene_st:901885- | 9.28E−15 | 2.4869 | 2.37 | 89.3 | 82.3-94.1 |
| | HuGene_st:421400-HuGene_st:835589- | | | | | |
| | HuGene_st:70935-HuGene_st:462061- | | | | | |
| | HuGene_st:702702-HuGene_st:719818- | | | | | |
| | HuGene_st:766065-HuGene_st:612065- | | | | | |
| | HuGene_st:825595-HuGene_st:1004265- | | | | | |
| | HuGene_st:206488-HuGene_st:867231- | | | | | |
| | HuGene_st:858503-HuGene_st:29674- | | | | | |
| | HuGene_st:26237-HuGene_st:351356- | | | | | |
| | HuGene_st:52388-HuGene_st:192518-HuGene_st | | | | | |
| RPL24 | 1559655_at:1559656_a_at:228885_at | 1.23E−14 | 2.5579 | 1.63 | 90 | 83.1-94.6 |
| CXCL12 | 567389-HuGene_st:886750-HuGene_st:815979- | 1.54E−14 | 2.4343 | 3.79 | 88.8 | 81.6-93.7 |
| | HuGene_st:1018943-HuGene_st:209687_at:800403- | | | | | |
| | HuGene_st:500331-HuGene_st:369373- | | | | | |
| | HuGene_st:1050367-HuGene_st:952256- | | | | | |
| | HuGene_st:1066302-HuGene_st:448047- | | | | | |
| | HuGene_st:186655-HuGene_st:771363- | | | | | |
| | HuGene_st:103922-HuGene_st:1025591- | | | | | |
| | HuGene_st:47442-HuGene_st:624854- | | | | | |
| | HuGene_st:211406-HuGene_st:255389-HuGene_st | | | | | |
| C1orf115 | 293810-HuGene_st:439869-HuGene_st:640378- | 1.95E−14 | 2.4078 | 2.95 | 88.6 | 81.4-93.6 |
| | HuGene_st:226391-HuGene_st:88026- | | | | | |
| | HuGene_st:230159_at:709535-HuGene_st:687885- | | | | | |
| | HuGene_st:218546_at:628297-HuGene_st:695536- | | | | | |
| | HuGene_st:230774-HuGene_st:884109- | | | | | |
| | HuGene_st:517531-HuGene_st:472518- | | | | | |
| | HuGene_st:8635-HuGene_st:158841-HuGene_st | | | | | |
| SLC26A2 | 224963_at:861453-HuGene_st:175674- | 2.08E−14 | 2.4223 | 7.83 | 88.7 | 81.5-93.7 |
| | HuGene_st:658865-HuGene_st:979969- | | | | | |
| | HuGene_st:1053352-HuGene_st:224959_at:509013- | | | | | |
| | HuGene_st:755618-HuGene_st:879968- | | | | | |
| | HuGene_st:205097_at:901728-HuGene_st:975514- | | | | | |
| | HuGene_st:407147-HuGene_st:891478- | | | | | |
| | HuGene_st:196344-HuGene_st:1015698- | | | | | |
| | HuGene_st:955403-HuGene_st:430227- | | | | | |
| | HuGene_st:1075936-HuGene_st:1080935- | | | | | |
| | HuGene_st:18346-HuGene_st:341007- | | | | | |
| | HuGene_st:471383-HuGene_st | | | | | |
| MEIS1 | 868613-HuGene_st:1072855-HuGene_st:139075- | 2.49E−14 | 2.3774 | 5.44 | 88.3 | 81-93.4 |
| | HuGene_st:721526-HuGene_st:1559477_s_at:381698- | | | | | |
| | HuGene_st:641395-HuGene_st:494458- | | | | | |
| | HuGene_st:183063-HuGene_st:212378- | | | | | |
| | HuGene_st:579377-HuGene_st:1006382- | | | | | |
| | HuGene_st:753093-HuGene_st:863677- | | | | | |
| | HuGene_st:517122-HuGene_st:777085- | | | | | |
| | HuGene_st:386678-HuGene_st:204069_at:1005832- | | | | | |
| | HuGene_st:395572-HuGene_st:231941- | | | | | |
| | HuGene_st:568986-HuGene_st:108139-HuGene_st | | | | | |
| SFRP2 | 144787-HuGene_st:337246-HuGene_st:97123- | 3.05E−14 | 2.3944 | 7.39 | 88.4 | 81.2-93.5 |
| | HuGene_st:614920-HuGene_st:223121_s_at:236674- | | | | | |
| | HuGene_st:602699-HuGene_st:370588- | | | | | |
| | HuGene_st:794693-HuGene_st:252957- | | | | | |
| | HuGene_st:870849-HuGene_st:279759- | | | | | |
| | HuGene_st:367084-HuGene_st:691080- | | | | | |
| | HuGene_st:223122_s_at:466709-HuGene_st | | | | | |
| TUBB6 | 209191_at:238151_at:267951-HuGene_st:578028- | 3.55E−14 | 2.3694 | 5.9 | 88.2 | 80.9-93.3 |
| | HuGene_st:862750-HuGene_st | | | | | |
| GPX3 | 623237-HuGene_st:40650-HuGene_st:134873- | 5.68E−14 | 2.3613 | 2.57 | 88.1 | 80.8-93.2 |
| | HuGene_st:952394-HuGene_st:644367- | | | | | |
| | HuGene_st:428176- | | | | | |
| | HuGene_st:201348_at:214091_s_at:377060- | | | | | |
| | HuGene_st:674301-HuGene_st:142325- | | | | | |
| | HuGene_st:905944-HuGene_st:439631- | | | | | |
| | HuGene_st:1064572-HuGene_st:587834- | | | | | |
| | HuGene_st:1040504-HuGene_st:250733- | | | | | |
| | HuGene_st:199701-HuGene_st:784815- | | | | | |
| | HuGene_st:652659-HuGene_st | | | | | |
| CD36 | 392196-HuGene_st:274514-HuGene_st:477005- | 6.27E−14 | 2.4677 | 1.65 | 89.1 | 82.1-94 |
| | HuGene_st:691585-HuGene_st:872909- | | | | | |
| | HuGene_st:543050-HuGene_st:603343- | | | | | |
| | HuGene_st:514557-HuGene_st:296850- | | | | | |
| | HuGene_st:945913-HuGene_st:495755- | | | | | |
| | HuGene_st:206488_s_at:1035854-HuGene_st:887301- | | | | | |
| | HuGene_st:836370-HuGene_st:209555_s_at:939919- | | | | | |
| | HuGene_st:507440-HuGene_st:151788- | | | | | |
| | HuGene_st:146280-HuGene_st:360545- | | | | | |
| | HuGene_st:1051486-HuGene_st:228766_at:512885- | | | | | |
| | HuGene_st | | | | | |
| HIGD1A | 936261-HuGene_st:824654-HuGene_st:165981- | 6.29E−14 | 2.4011 | 2.09 | 88.5 | 81.3-93.5 |
| | HuGene_st:225595-HuGene_st:290747- | | | | | |
| | HuGene_st:641007-HuGene_st:1073341- | | | | | |
| | HuGene_st:761357-HuGene_st:818902- | | | | | |
| | HuGene_st:1038575-HuGene_st:102828- | | | | | |
| | HuGene_st:533707-HuGene_st:414059- | | | | | |
| | HuGene_st:217845_x_at:852712- | | | | | |
| | HuGene_st:221896_s_at:291015-HuGene_st:295866- | | | | | |
| | HuGene_st:520869:HuGene_st:155393- | | | | | |
| | HuGene_st:460566-HuGene_st:969731- | | | | | |
| | HuGene_st:170508-HuGene_st:744650- | | | | | |
| | HuGene_st:309982-HuGene_st:795342- | | | | | |
| | HuGene_st:387733-HuGene_st:324047- | | | | | |
| | HuGene_st:239559-HuGene_st:1055553- | | | | | |
| | HuGene_st:827869-HuGene_st:1058468-HuGene_st | | | | | |
| NR3C1 | 111450-HuGene_st:434945-HuGene_st:1054045- | 7.65E−14 | 2.3495 | 2.51 | 88 | 80.6-93.2 |
| | HuGene_st:201866_s_at:98168-HuGene_st:402326- | | | | | |
| | HuGene_st:154154-HuGene_st:117026- | | | | | |
| | HuGene_st:1027472-HuGene_st:517524- | | | | | |
| | HuGene_st:540910-HuGene_st:538776- | | | | | |
| | HuGene_st:550290-HuGene_st:460796- | | | | | |
| | HuGene_st:211671_s_at:216321_s_at:201865_x_at:649431- | | | | | |
| | HuGene_st:461795-HuGene_st | | | | | |
| LOC63928 | 206149_at:681928-HuGene_st:1004459- | 8.09E−14 | 2.352 | 3.2 | 88 | 80.6-93.2 |
| | HuGene_st:249694-HuGene_st:80283- | | | | | |
| | HuGene_st:323497-HuGene_st:650243- | | | | | |
| | HuGene_st:872231-HuGene_st:253533- | | | | | |
| | HuGene_st:270238-HuGene_st:282291- | | | | | |
| | HuGene_st:268597-HuGene_st:913922- | | | | | |
| | HuGene_st:416350-HuGene_st:992808- | | | | | |
| | HuGene_st:472127-HuGene_st:988731- | | | | | |
| | HuGene_st:243977-HuGene_st:802623- | | | | | |
| | HuGene_st:314649-HuGene_st | | | | | |
| MXD1 | 226275_at:206877_at:602969-HuGene_st:127172- | 8.35E−14 | 2.4077 | 2.11 | 88.6 | 81.3-93.6 |
| | HuGene_st:577694-HuGene_st:53257- | | | | | |
| | HuGene_st:228846_at:669193-HuGene_st:37027- | | | | | |
| | HuGene_st:458872-HuGene_st:518444- | | | | | |
| | HuGene_st:410159-HuGene_st:77113- | | | | | |
| | HuGene_st:280760-HuGene_st:229252- | | | | | |
| | HuGene_st:318164-HuGene_st:393130- | | | | | |
| | HuGene_st:172809-HuGene_st:1020416- | | | | | |
| | HuGene_st:1050501-HuGene_st:269397- | | | | | |
| | HuGene_st:68122-HuGene_st:423400- | | | | | |
| | HuGene_st:103208-HuGene_st:936311-HuGene_st | | | | | |
| C9orf19 | 268503-HuGene_st:209268- | 1.74E−13 | 2.3659 | 1.95 | 88.2 | 80.9-93.3 |
| | HuGene_st:225604_s_at:518559-HuGene_st:1007612- | | | | | |
| | HuGene_st:499070-HuGene_st:225602_at:291028- | | | | | |
| | HuGene_st:615472-HuGene_st:3136- | | | | | |
| | HuGene_st:926635-HuGene_st:219003- | | | | | |
| | HuGene_st:396117-HuGene_st:94077- | | | | | |
| | HuGene_st:747558-HuGene_st:226948- | | | | | |
| | HuGene_st:940376-HuGene_st:977640- | | | | | |
| | HuGene_st:316643-HuGene_st:382182- | | | | | |
| | HuGene_st:745966-HuGene_st | | | | | |
| DCN | 6640-HuGene_st:201893_x_at:211813_x_at:311031- | 2.05E−13 | 2.3127 | 5.62 | 87.6 | 80.3-92.9 |
| | HuGene_st:211896_s_at:505982-HuGene_st:219729- | | | | | |
| | HuGene_st:1067446-HuGene_st:538017- | | | | | |
| | HuGene_st:13085-HuGene_st:960643- | | | | | |
| | HuGene_st:132704-HuGene_st:253410- | | | | | |
| | HuGene_st:54359-HuGene_st:1020632- | | | | | |
| | HuGene_st:973501-HuGene_st:483405- | | | | | |
| | HuGene_st:252884-HuGene_st:274013- | | | | | |
| | HuGene_st:664014-HuGene_st:522817- | | | | | |
| | HuGene_st:745324-HuGene_st:209335_at:339470- | | | | | |
| | HuGene_st:916917-HuGene_st | | | | | |
| HSD17B2 | 281748-HuGene_st:142500-HuGene_st:602472- | 2.32E−13 | 2.1956 | 4.76 | 86.4 | 78.7-92 |
| | HuGene_st:437526-HuGene_st:1057583- | | | | | |
| | HuGene_st:999857-HuGene_st:915850- | | | | | |
| | HuGene_st:1097510-HuGene_st:127085- | | | | | |
| | HuGene_st:1088665-HuGene_st:194898- | | | | | |
| | HuGene_st:834134-HuGene_st:360927- | | | | | |
| | HuGene_st:1029542-HuGene_st:422429- | | | | | |
| | HuGene_st:573141-HuGene_st:204818_at:918446- | | | | | |
| | HuGene_st:386936-HuGene_st:613850- | | | | | |
| | HuGene_st:489258-HuGene_st | | | | | |
| TCF21 | 804657-HuGene_st:106365-HuGene_st:710514- | 2.51E−13 | 2.3047 | 2.34 | 87.5 | 80.1-92.8 |
| | HuGene_st:299556-HuGene_st:608149- | | | | | |
| | HuGene_st:242233-HuGene_st:655881- | | | | | |
| | HuGene_st:356773-HuGene_st:788445- | | | | | |
| | HuGene_st:709897-HuGene_st:605488- | | | | | |
| | HuGene_st:652466-HuGene_st:204931_at:830709- | | | | | |
| | HuGene_st:273418-HuGene_st:1004754- | | | | | |
| | HuGene_st:990861-HuGene_st:238739- | | | | | |
| | HuGene_st:836241-HuGene_st:110045- | | | | | |
| | HuGene_st:229529_at:1001969-HuGene_st | | | | | |
| MRC1 | 467197-HuGene_st:758500-HuGene_st:600690- | 2.72E−13 | 2.3106 | 2.6 | 87.6 | 80.2-92.9 |
| | HuGene_st:270842-HuGene_st:787475- | | | | | |
| | HuGene_st:710055-HuGene_st:295499- | | | | | |
| | HuGene_st:757670-HuGene_st:994062- | | | | | |
| | HuGene_st:587369-HuGene_st:224499- | | | | | |
| | HuGene_st:428438-HuGene_st:577964- | | | | | |
| | HuGene_st:159156-HuGene_st:298439- | | | | | |
| | HuGene_st:1088275-HuGene_st:715300- | | | | | |
| | HuGene_st:1627-HuGene_st:513230- | | | | | |
| | HuGene_st:106268-HuGene_st:204438_at | | | | | |
| ADH1C | 661427-HuGene_st:243544_at:634797- | 2.94E−13 | 2.3724 | 3.1 | 88.2 | 81-93.3 |
| | HuGene_st:291687-HuGene_st:972177- | | | | | |
| | HuGene_st:316339-HuGene_st:580686- | | | | | |
| | HuGene_st:206262_at:211851-HuGene_st:641619- | | | | | |
| | HuGene_st:142959-HuGene_st:705200- | | | | | |
| | HuGene_st:1063529-HuGene_st:916148- | | | | | |
| | HuGene_st:901368-HuGene_st:43611- | | | | | |
| | HuGene_st:789755-HuGene_st:410220- | | | | | |
| | HuGene_st:289785-HuGene_st:155724- | | | | | |
| | HuGene_st:289958-HuGene_st:232595-HuGene_st | | | | | |
| BCHE | 228090-HuGene_st:752051-HuGene_st:800167- | 3.18E−13 | 2.2535 | 6.21 | 87 | 79.4-92.4 |
| | HuGene_st:221362-HuGene_st:155900- | | | | | |
| | HuGene_st:717363-HuGene_st:536584- | | | | | |
| | HuGene_st:146780-HuGene_st:302487- | | | | | |
| | HuGene_st:508472-HuGene_st:516293- | | | | | |
| | HuGene_st:968992-HuGene_st:666625- | | | | | |
| | HuGene_st:923158-HuGene_st | | | | | |
| CCDC80 | 1072039-HuGene_st:973747-HuGene_st:580839- | 3.60E−13 | 2.2322 | 3.67 | 86.8 | 79.2-92.3 |
| | HuGene_st:583669-HuGene_st:290210- | | | | | |
| | HuGene_st:308086-HuGene_st:679211- | | | | | |
| | HuGene_st:43315-HuGene_st:1080603- | | | | | |
| | HuGene_st:852141-HuGene_st:569200- | | | | | |
| | HuGene_st:92231-HuGene_st:535306- | | | | | |
| | HuGene_st:427093-HuGene_st:1003098- | | | | | |
| | HuGene_st:408541-HuGene_st:135243- | | | | | |
| | HuGene_st:225298- | | | | | |
| | HuGene_st:225242_s_at:225241_at:633079- | | | | | |
| | HuGene_st:43353-HuGene_st:642388- | | | | | |
| | HuGene_st:243864_at | | | | | |
| CITED2 | 125201-HuGene_st:410723-HuGene_st:463405- | 5.29E−13 | 2.2712 | 1.64 | 87.2 | 79.7-92.6 |
| | HuGene_st:401168-HuGene_st:1012057- | | | | | |
| | HuGene_st:235057-HuGene_st:361772- | | | | | |
| | HuGene_st:207980_s_at:1091907-HuGene_st:985355- | | | | | |
| | HuGene_st:175990-HuGene_st:227287_at:48433- | | | | | |
| | HuGene_st:209357_at:477746-HuGene_st:243264- | | | | | |
| | HuGene_st:904401-HuGene_st:328536- | | | | | |
| | HuGene_st:1095110-HuGene_st:89784- | | | | | |
| | HuGene_st:206734-HuGene_st:927615-HuGene_st | | | | | |
| PCK1 | 208383_s_at:221954-HuGene_st:772877- | 5.74E−13 | 2.3236 | 3.6 | 87.7 | 80.4-93 |
| | HuGene_st:518166-HuGene_st:1098164- | | | | | |
| | HuGene_st:241175-HuGene_st:111987- | | | | | |
| | HuGene_st:395761-HuGene_st:846308- | | | | | |
| | HuGene_st:584532-HuGene_st:547720- | | | | | |
| | HuGene_st:1018765-HuGene_st:817976- | | | | | |
| | HuGene_st:454213-HuGene_st:66958- | | | | | |
| | HuGene_st:301878-HuGene_st:804419- | | | | | |
| | HuGene_st:442879-HuGene_st:211870- | | | | | |
| | HuGene_st:638998-HuGene_st:448080- | | | | | |
| | HuGene_st:948845-HuGene_st:313770-HuGene_st | | | | | |
| ANK3 | 472262-HuGene_st:337923-HuGene_st:715126- | 7.46E−13 | 2.2325 | 2.45 | 86.8 | 79.2-92.3 |
| | HuGene_st:416201-HuGene_st:942091- | | | | | |
| | HuGene_st:364373- | | | | | |
| | HuGene_st:207950_s_at:209442_x_at:410536- | | | | | |
| | HuGene_st:206385_s_at:845022-HuGene_st:400240- | | | | | |
| | HuGene_st:651770-HuGene_st:29904- | | | | | |
| | HuGene_st:215314_at | | | | | |
| F13A1 | 412872-HuGene_st:755197-HuGene_st:1023946- | 8.82E−13 | 2.1855 | 4.74 | 86.3 | 78.6-91.9 |
| | HuGene_st:591121-HuGene_st:912274- | | | | | |
| | HuGene_st:465239-HuGene_st:1002989- | | | | | |
| | HuGene_st:541053-HuGene_st:1052450- | | | | | |
| | HuGene_st:204339-HuGene_st:176807- | | | | | |
| | HuGene_st:58339-HuGene_st:42656- | | | | | |
| | HuGene_st:965853-HuGene_st:1096456- | | | | | |
| | HuGene_st:365069-HuGene_st:300786- | | | | | |
| | HuGene_st:739769-HuGene_st:70605- | | | | | |
| | HuGene_st:174382-HuGene_st:203305_at:783884- | | | | | |
| | HuGene_st | | | | | |
| ADAMDEC1 | 893574-HuGene_st:1087946-HuGene_st:453119- | 9.04E−13 | 2.2604 | 3.19 | 87.1 | 79.5-92.5 |
| | HuGene_st:995166-HuGene_st:660805- | | | | | |
| | HuGene_st:162940-HuGene_st:402188- | | | | | |
| | HuGene_st:782577-HuGene_st:89831- | | | | | |
| | HuGene_st:11993-HuGene_st:1020874- | | | | | |
| | HuGene_st:997719-HuGene_st:268541- | | | | | |
| | HuGene_st:41641-HuGene_st:1004377- | | | | | |
| | HuGene_st:53970-HuGene_st:206134_at:904943- | | | | | |
| | HuGene_st:644140-HuGene_st:332803- | | | | | |
| | HuGene_st:16627-HuGene_st:1555-HuGene_st | | | | | |
| ABI3BP | 801423-HuGene_st:1065486-HuGene_st:692353- | 9.47E−13 | 2.2176 | 3.62 | 86.6 | 79-92.2 |
| | HuGene_st:456260-HuGene_st:686533- | | | | | |
| | HuGene_st:68008-HuGene_st:317350- | | | | | |
| | HuGene_st:239950-HuGene_st:604210- | | | | | |
| | HuGene_st:20407-HuGene_st:1099546- | | | | | |
| | HuGene_st:395466-HuGene_st:548603- | | | | | |
| | HuGene_st:931079-HuGene_st:159325- | | | | | |
| | HuGene_st:251067-HuGene_st:1056116- | | | | | |
| | HuGene_st:224253-HuGene_st:885069- | | | | | |
| | HuGene_st:1075816-HuGene_st:592859-HuGene_st | | | | | |
| PAG1 | 572231-HuGene_st:431317-HuGene_st:342766- | 1.11E−12 | 2.215 | 2.68 | 86.6 | 79-92.1 |
| | HuGene_st:1080197-HuGene_st:225622_at:81123- | | | | | |
| | HuGene_st:859237-HuGene_st:287765- | | | | | |
| | HuGene_st:225626_at:743424-HuGene_st:759835- | | | | | |
| | HuGene_st:622195-HuGene_st:208082- | | | | | |
| | HuGene_st:768207-HuGene_st:657358- | | | | | |
| | HuGene_st:506609-HuGene_st:226184- | | | | | |
| | HuGene_st:540710-HuGene_st:236888- | | | | | |
| | HuGene_st:36408-HuGene_st:546382- | | | | | |
| | HuGene_st:498489-HuGene_st:227354_at | | | | | |
| KRT20 | 11827-HuGene_st:50958-HuGene_st:684746- | 1.26E−12 | 2.0981 | 5.49 | 85.3 | 77.4-91.1 |
| | HuGene_st:574501-HuGene_st:270813- | | | | | |
| | HuGene_st:421213-HuGene_st:213953_at:225474- | | | | | |
| | HuGene_st:310071-HuGene_st:356429- | | | | | |
| | HuGene_st:997077-HuGene_st:963515- | | | | | |
| | HuGene_st:944854-HuGene_st:178191- | | | | | |
| | HuGene_st:613655-HuGene_st:337174- | | | | | |
| | HuGene_st:268017-HuGene_st:81041- | | | | | |
| | HuGene_st:85506-HuGene_st:798272- | | | | | |
| | HuGene_st:925916-HuGene_st:780714- | | | | | |
| | HuGene_st:138025-HuGene_st | | | | | |
| ENTPD5 | 760608-HuGene_st:231676_s_at:831252- | 1.43E−12 | 2.1686 | 3.01 | 86.1 | 78.3-91.7 |
| | HuGene_st:497317-HuGene_st:712688- | | | | | |
| | HuGene_st:259180-HuGene_st:832617- | | | | | |
| | HuGene_st:785663-HuGene_st:205757_at:553118- | | | | | |
| | HuGene_st:384386-HuGene_st:911183- | | | | | |
| | HuGene_st:440835-HuGene_st:150070- | | | | | |
| | HuGene_st:821276-HuGene_st:14461- | | | | | |
| | HuGene_st:520930-HuGene_st:181407- | | | | | |
| | HuGene_st:294287-HuGene_st:226594_at:551172- | | | | | |
| | HuGene_st:1040872-HuGene_st:1048020-HuGene_st | | | | | |
| MALL | 209373_at:628866-HuGene_st:1092892- | 2.10E−12 | 2.1585 | 3.96 | 86 | 78.2-91.7 |
| | HuGene_st:488187-HuGene_st:515617- | | | | | |
| | HuGene_st:649198-HuGene_st:991653- | | | | | |
| | HuGene_st:301852-HuGene_st:1067925- | | | | | |
| | HuGene_st:451072-HuGene_st:102723- | | | | | |
| | HuGene_st:670639-HuGene_st:848482- | | | | | |
| | HuGene_st:928502-HuGene_st:787192- | | | | | |
| | HuGene_st:916920-HuGene_st:959742- | | | | | |
| | HuGene_st:711672-HuGene_st:20252- | | | | | |
| | HuGene_st:225337-HuGene_st | | | | | |
| MFAP4 | 308095-HuGene_st:1004622-HuGene_st:754788- | 2.96E−12 | 2.1813 | 2.51 | 86.2 | 78.5-91.9 |
| | HuGene_st:212713_at:911082-HuGene_st:767889- | | | | | |
| | HuGene_st:5569-HuGene_st:308458- | | | | | |
| | HuGene_st:699815-HuGene_st:658908- | | | | | |
| | HuGene_st:73048-HuGene_st:515283- | | | | | |
| | HuGene_st:671512-HuGene_st:792571- | | | | | |
| | HuGene_st:689375-HuGene_st:590963- | | | | | |
| | HuGene_st:821074-HuGene_st:132543- | | | | | |
| | HuGene_st:655171-HuGene_st:341975- | | | | | |
| | HuGene_st:1048103-HuGene_st | | | | | |
| SRI | 1018107-HuGene_st:929936-HuGene_st:596109- | 3.03E−12 | 2.0985 | 2.43 | 85.3 | 77.4-91.1 |
| | HuGene_st:469173-HuGene_st:235900- | | | | | |
| | HuGene_st:303619-HuGene_st:792372- | | | | | |
| | HuGene_st:1034405-HuGene_st:145638- | | | | | |
| | HuGene_st:76828-HuGene_st:103032- | | | | | |
| | HuGene_st:788729-HuGene_st:262807- | | | | | |
| | HuGene_st:452805-HuGene_st:208921_s_at:863794- | | | | | |
| | HuGene_st:172167-HuGene_st:309679- | | | | | |
| | HuGene_st:1058573-HuGene_st:208920_at:235325- | | | | | |
| | HuGene_st:813384-HuGene_st | | | | | |
| SGK | 425210-HuGene_st:201739_at:137804- | 3.12E−12 | 2.1186 | 4.08 | 85.5 | 77.7-91.4 |
| | HuGene_st:182666-HuGene_st:271994- | | | | | |
| | HuGene_st:629671-HuGene_st:350855- | | | | | |
| | HuGene_st:179953-HuGene_st:1039396- | | | | | |
| | HuGene_st:664449-HuGene_st:1049246- | | | | | |
| | HuGene_st:1100037-HuGene_st:1094410- | | | | | |
| | HuGene_st:677142-HuGene_st:231137- | | | | | |
| | HuGene_st:393473-HuGene_st | | | | | |
| IGHM | 1001583-HuGene_st:162285-HuGene_st:1061429- | 3.25E−12 | 2.3285 | 1.73 | 87.8 | 80.4-93 |
| | HuGene_st:359832-HuGene_st:60182: | | | | | |
| | HuGene_st:1002550-HuGene_st:311771- | | | | | |
| | HuGene_st:790352-HuGene_st:977059- | | | | | |
| | HuGene_st:587401-HuGene_st:343597- | | | | | |
| | HuGene_st:25657-HuGene_st:640259- | | | | | |
| | HuGene_st:184414-HuGene_st:336659- | | | | | |
| | HuGene_st:1008406-HuGene_st:1044209- | | | | | |
| | HuGene_st:519020-HuGene_st:427467- | | | | | |
| | HuGene_st:669553-HuGene_st:722843- | | | | | |
| | HuGene_st:126162-HuGene_st:654578- | | | | | |
| | HuGene_st:146515-HuGene_st:1100721- | | | | | |
| | HuGene_st:823307-HuGene_st:899494- | | | | | |
| | HuGene_st:952685-HuGene_st:215118_s_at:235399- | | | | | |
| | HuGene_st:802521-HuGene_st:126175- | | | | | |
| | HuGene_st:910847-HuGene_st:212827_at:722700- | | | | | |
| | HuGene_st:173623-HuGene_st:1042953- | | | | | |
| | HuGene_st:38807-HuGene_st:408026-HuGene_st | | | | | |
| ZCWPW2 | 1001583-HuGene_st:162285-HuGene_st:1061429- | 3.25E−12 | 2.3264 | 1.73 | 87.8 | 80.4-93 |
| | HuGene_st:359832-HuGene_st:60182- | | | | | |
| | HuGene_st:1002550-HuGene_st:311771- | | | | | |
| | HuGene_st:790352-HuGene_st:977059- | | | | | |
| | HuGene_st:587401-HuGene_st:343597- | | | | | |
| | HuGene_st:25657-HuGene_st:640259- | | | | | |
| | HuGene_st:184414-HuGene_st:336659- | | | | | |
| | HuGene_st:1008406-HuGene_st:1044209- | | | | | |
| | HuGene_st:519020-HuGene_st:427467- | | | | | |
| | HuGene_st:669553-HuGene_st:722843- | | | | | |
| | HuGene_st:126162-HuGene_st:654578- | | | | | |
| | HuGene_st:146515-HuGene_st:1100721- | | | | | |
| | HuGene_st:823307-HuGene_st:899494- | | | | | |
| | HuGene_st:952685-HuGene_st:215118_s_at:235399- | | | | | |
| | HuGene_st:802521-HuGene_st:126175- | | | | | |
| | HuGene_st:910847-HuGene_st:212827_at:722700- | | | | | |
| | HuGene_st:173623-HuGene_st:1042953- | | | | | |
| | HuGene_st:38807-HuGene_st:408026-HuGene_st | | | | | |
| IL8 | 1001583-HuGene_st:162285-HuGene_st:1061429- | 3.28E−12 | 2.3266 | 1.73 | 87.8 | 80.4-93 |
| | HuGene_st:359832-HuGene_st:60182- | | | | | |
| | HuGene_st:1002550-HuGene_st:311771- | | | | | |
| | HuGene_st:790352-HuGene_st:977059- | | | | | |
| | HuGene_st:587401-HuGene_st:343597- | | | | | |
| | HuGene_st:25657-HuGene_st:640259- | | | | | |
| | HuGene_st:184414-HuGene_st:336659- | | | | | |
| | HuGene_st:1008406-HuGene_st:1044209- | | | | | |
| | HuGene_st:519020-HuGene_st:427467- | | | | | |
| | HuGene_st:669553-HuGene_st:722843- | | | | | |
| | HuGene_st:126162-HuGene_st:654578- | | | | | |
| | HuGene_st:146515-HuGene_st:1100721- | | | | | |
| | HuGene_st:823307-HuGene_st:899494- | | | | | |
| | HuGene_st:952685-HuGene_st:215118_s_at:235399- | | | | | |
| | HuGene_st:802521-HuGene_st:126175- | | | | | |
| | HuGene_st:910847-HuGene_st:212827_at:722700- | | | | | |
| | HuGene_st:173623-HuGene_st:1042953- | | | | | |
| | HuGene_st:38807-HuGene_st:408026-HuGene_st | | | | | |
| IGHA1 | 1001583-HuGene_st:162285-HuGene_st:1061429- | 3.28E−12 | 2.3276 | 1.73 | 87.8 | 80.4-93 |
| | HuGene_st:359832-HuGene_st:60182- | | | | | |
| | HuGene_st:1002550-HuGene_st:311771- | | | | | |
| | HuGene_st:790352-HuGene_st:977059- | | | | | |
| | HuGene_st:587401-HuGene_st:343597- | | | | | |
| | HuGene_st:25657-HuGene_st:640259- | | | | | |
| | HuGene_st:184414-HuGene_st:336659- | | | | | |
| | HuGene_st:1008406-HuGene_st:235364_at:1044209- | | | | | |
| | HuGene_st:519020-HuGene_st:427467- | | | | | |
| | HuGene_st:669553-HuGene_st:722843- | | | | | |
| | HuGene_st:208763_s_at:207001_x_at:126162- | | | | | |
| | HuGene_st:654578-HuGene_st:146515- | | | | | |
| | HuGene_st:1100721-HuGene_st:823307- | | | | | |
| | HuGene_st:899494-HuGene_st:952685- | | | | | |
| | HuGene_st:215118_s_at:235399-HuGene_st:802521- | | | | | |
| | HuGene_st:126175-HuGene_st:910847- | | | | | |
| | HuGene_st:212827_at:722700-HuGene_st:173623- | | | | | |
| | HuGene_st:1042953-HuGene_st:38807- | | | | | |
| | HuGene_st:408026-HuGene_st | | | | | |
| MGC14376 | 20793-HuGene_st:228915-HuGene_st:630688- | 3.39E−12 | 2.1014 | 2.58 | 85.3 | 77.5-91.2 |
| | HuGene_st:117325-HuGene_st:532108- | | | | | |
| | HuGene_st:373670-HuGene_st:552164- | | | | | |
| | HuGene_st:1004615-HuGene_st:527421- | | | | | |
| | HuGene_st:880155-HuGene_st:10973- | | | | | |
| | HuGene_st:395595-HuGene_st:312241- | | | | | |
| | HuGene_st:644241-HuGene_st:214696_at:234825- | | | | | |
| | HuGene_st:1002029-HuGene_st:1032648-HuGene_st | | | | | |
| ETHE1 | 1008653-HuGene_st:507181-HuGene_st:435886- | 3.46E−12 | 2.2097 | 1.75 | 86.5 | 78.9-92.1 |
| | HuGene_st:296660-HuGene_st:764234- | | | | | |
| | HuGene_st:918966-HuGene_st:1078682- | | | | | |
| | HuGene_st:789091-HuGene_st:100698- | | | | | |
| | HuGene_st:204034_at:301334-HuGene_st:869871- | | | | | |
| | HuGene_st:841094-HuGene_st:612751- | | | | | |
| | HuGene_st:43763-HuGene_st:853527- | | | | | |
| | HuGene_st:593768-HuGene_st:52787- | | | | | |
| | HuGene_st:555225-HuGene_st:1028299-HuGene_st | | | | | |
| CES2 | 817786-HuGene_st:63720-HuGene_st:527565- | 3.51E−12 | 2.0993 | 2.87 | 85.3 | 77.4-91.1 |
| | HuGene_st:678893-HuGene_st:209668_x_at:825633- | | | | | |
| | HuGene_st:336873-HuGene_st:409854- | | | | | |
| | HuGene_st:213509_x_at:134988-HuGene_st:446296- | | | | | |
| | HuGene_st:842160-HuGene_st:827636- | | | | | |
| | HuGene_st:209667_at:882043-HuGene_st:279172- | | | | | |
| | HuGene_st:1081267-HuGene_st:756682- | | | | | |
| | HuGene_st:90632-HuGene_st | | | | | |
| GPM6B | 599862-HuGene_st:754598-HuGene_st:503642- | 4.04E−12 | 2.1563 | 3.59 | 86 | 78.2-91.6 |
| | HuGene_st:224935-HuGene_st:754449- | | | | | |
| | HuGene_st:577489-HuGene_st:1073242- | | | | | |
| | HuGene_st:560873-HuGene_st:1003662- | | | | | |
| | HuGene_st:430217-HuGene_st:903323- | | | | | |
| | HuGene_st:231962-HuGene_st:244945- | | | | | |
| | HuGene_st:562460-HuGene_st:583561- | | | | | |
| | HuGene_st:209168_at | | | | | |
| SYNPO2 | 800385-HuGene_st:938576-HuGene_st:54552- | 5.07E−12 | 2.1029 | 6.43 | 85.3 | 77.5-91.2 |
| | HuGene_st:946854-HuGene_st:278208- | | | | | |
| | HuGene_st:170777-HuGene_st:796143- | | | | | |
| | HuGene_st:544243-HuGene_st:469048- | | | | | |
| | HuGene_st:364454-HuGene_st:400491- | | | | | |
| | HuGene_st:22204-HuGene_st:315299- | | | | | |
| | HuGene_st:526384-HuGene_st:841867- | | | | | |
| | HuGene_st:244108_at:2158-HuGene_st:283531- | | | | | |
| | HuGene_st:407855- | | | | | |
| | HuGene_st:225721_at:225720_at:225895_at:225894_at: | | | | | |
| | 1017102-HuGene_st:227662_at:232119_at | | | | | |
| GCG | 1044997-HuGene_st:682227-HuGene_st:368693- | 5.58E−12 | 2.1165 | 12.56 | 85.5 | 77.7-91.3 |
| | HuGene_st:25836-HuGene_st:228935- | | | | | |
| | HuGene_st:1030502-HuGene_st:12466- | | | | | |
| | HuGene_st:1098133-HuGene_st:994392- | | | | | |
| | HuGene_st:581476-HuGene_st:799068- | | | | | |
| | HuGene_st:350795-HuGene_st:705389- | | | | | |
| | HuGene_st:981135-HuGene_st:736837- | | | | | |
| | HuGene_st:253854-HuGene_st:1032660- | | | | | |
| | HuGene_st:273941-HuGene_st:206422_at:940866- | | | | | |
| | HuGene_st:514731-HuGene_st | | | | | |
| MGP | 696658-HuGene_st:618386- | 6.11E−12 | 2.1428 | 4.68 | 85.8 | 78-91.5 |
| | HuGene_st:202291_s_at:195765-HuGene_st:3486- | | | | | |
| | HuGene_st:829319-HuGene_st:731815- | | | | | |
| | HuGene_st:829950-HuGene_st:601468- | | | | | |
| | HuGene_st:366689-HuGene_st:460503- | | | | | |
| | HuGene_st:712045-HuGene_st:545937- | | | | | |
| | HuGene_st:148839-HuGene_st:704906- | | | | | |
| | HuGene_st:612318-HuGene_st:708651- | | | | | |
| | HuGene_st:722064-HuGene_st:846497- | | | | | |
| | HuGene_st:1084237-HuGene_st:854168-HuGene_st | | | | | |
| PDE9A | 779446-HuGene_st:768516-HuGene_st:609563- | 6.56E−12 | 2.145 | 3.91 | 85.8 | 78-91.5 |
| | HuGene_st:387613-HuGene_st:594122- | | | | | |
| | HuGene_st:774367-HuGene_st:221476- | | | | | |
| | HuGene_st:140410-HuGene_st:812534- | | | | | |
| | HuGene_st:581440-HuGene_st:205593_s_at:142832- | | | | | |
| | HuGene_st:767027-HuGene_st:464973- | | | | | |
| | HuGene_st:97182-HuGene_st:128487- | | | | | |
| | HuGene_st:206471-HuGene_st:878522- | | | | | |
| | HuGene_st:237139_at:988884-HuGene_st:237283_at | | | | | |
| LRRC19 | 177641-HuGene_st:1070020-HuGene_st:1055140- | 6.75E−12 | 2.0763 | 4.51 | 85 | 77.1-91 |
| | HuGene_st:525999-HuGene_st:937256- | | | | | |
| | HuGene_st:620791-HuGene_st:891251- | | | | | |
| | HuGene_st:707559-HuGene_st:892056- | | | | | |
| | HuGene_st:764919-HuGene_st:382143- | | | | | |
| | HuGene_st:52584-HuGene_st:920414- | | | | | |
| | HuGene_st:1028155-HuGene_st:755055- | | | | | |
| | HuGene_st:678651-HuGene_st:1080156- | | | | | |
| | HuGene_st:530282-HuGene_st:523877- | | | | | |
| | HuGene_st:335198-HuGene_st:787709- | | | | | |
| | HuGene_st:153175-HuGene_st:220376_at | | | | | |
| HSD11B2 | 78582-HuGene_st:653519-HuGene_st:630341- | 6.97E−12 | 2.0895 | 2.53 | 85.2 | 77.3-91.1 |
| | HuGene_st:919091-HuGene_st:132633- | | | | | |
| | HuGene_st:907106-HuGene_st:621993- | | | | | |
| | HuGene_st:1089280-HuGene_st:34729- | | | | | |
| | HuGene_st:342165-HuGene_st:887157- | | | | | |
| | HuGene_st:323862-HuGene_st:870050- | | | | | |
| | HuGene_st:204130_at:635792-HuGene_st:606173- | | | | | |
| | HuGene_st:867938-HuGene_st:113467- | | | | | |
| | HuGene_st:806665-HuGene_st | | | | | |
| GNG2 | 1555766_a_at:224965_at:1011730-HuGene_st:77527- | 7.25E−12 | 2.1437 | 1.91 | 85.8 | 78-91.5 |
| | HuGene_st:669616-HuGene_st:223943_s_at:938512- | | | | | |
| | HuGene_st:450519-HuGene_st:1025129- | | | | | |
| | HuGene_st:30927-HuGene_st:382458- | | | | | |
| | HuGene_st:330996-HuGene_st:545415- | | | | | |
| | HuGene_st:774093-HuGene_st:758152- | | | | | |
| | HuGene_st:176111-HuGene_st:743969-HuGene_st | | | | | |
| GCNT3 | 230376_at:254999-HuGene_st:476622- | 8.39E−12 | 2.0861 | 3.94 | 85.2 | 77.2-91 |
| | HuGene_st:219508_at:452671-HuGene_st:732602- | | | | | |
| | HuGene_st:306264-HuGene_st:696871- | | | | | |
| | HuGene_st:721027-HuGene_st:235453- | | | | | |
| | HuGene_st:676136-HuGene_st:777254- | | | | | |
| | HuGene_st:506598-HuGene_st:598423- | | | | | |
| | HuGene_st:699538-HuGene_st:220940- | | | | | |
| | HuGene_st:804174-HuGene_st:12527- | | | | | |
| | HuGene_st:802770-HuGene_st:403287- | | | | | |
| | HuGene_st:351721-HuGene_st:581784- | | | | | |
| | HuGene_st:700724-HuGene_st:398939-HuGene_st | | | | | |
| UGT1A6 | 116025-HuGene_st:6488-HuGene_st:881135- | 1.32E−11 | 2.07 | 4.56 | 85 | 77-90.9 |
| | HuGene_st:230953_at:221304_at:221305_s_at:511516- | | | | | |
| | HuGene_st:594963-HuGene_st:396121- | | | | | |
| | HuGene_st:123777-HuGene_st:1016481- | | | | | |
| | HuGene_st:204532_x_at:683377-HuGene_st:1055169- | | | | | |
| | HuGene_st:1035103-HuGene_st:1088102- | | | | | |
| | HuGene_st:207126_x_at:42874- | | | | | |
| | HuGene_st:215125_s_at:206094_x_at:208596_s_at:97211- | | | | | |
| | HuGene_st:1009861-HuGene_st:603368- | | | | | |
| | HuGene_st:232654_s_at:625897-HuGene_st:998604- | | | | | |
| | HuGene_st | | | | | |
| UGT1A8 | 116025-HuGene_st:6488-HuGene_st:881135- | 1.32E−11 | 2.0687 | 4.56 | 85 | 77-90.9 |
| | HuGene_st:230953_at:221304_at:221305_s_at:511516- | | | | | |
| | HuGene_st:594963-HuGene_st:396121- | | | | | |
| | HuGene_st:123777-HuGene_st:1016481- | | | | | |
| | HuGene_st:204532_x_at:683377-HuGene_st:1055169- | | | | | |
| | HuGene_st:1035103-HuGene_st:1088102- | | | | | |
| | HuGene_st:207126_x_at:42874- | | | | | |
| | HuGene_st:215125_s_at:206094_x_at:208596_s_at:97211- | | | | | |
| | HuGene_st:1009861-HuGene_st:603368- | | | | | |
| | HuGene_st:232654_s_at:625897-HuGene_st:998604- | | | | | |
| | HuGene_st | | | | | |
| UGT1A1 | 116025-HuGene_st:6488-HuGene_st:881135- | 1.32E−11 | 2.0661 | 4.56 | 84.9 | 77-90.8 |
| | HuGene_st:230953_at:221304_at:221305_s_at:511516- | | | | | |
| | HuGene_st:594963-HuGene_st:396121- | | | | | |
| | HuGene_st:123777-HuGene_st:1016481- | | | | | |
| | HuGene_st:204532_x_at:683377-HuGene_st:1055169- | | | | | |
| | HuGene_st:1035103-HuGene_st:1088102- | | | | | |
| | HuGene_st:207126_x_at:42874- | | | | | |
| | HuGene_st:215125_s_at:206094_x_at:208596_s_at:97211- | | | | | |
| | HuGene_st:1009861-HuGene_st:603368- | | | | | |
| | HuGene_st:232654_s_at:625897-HuGene_st:998604- | | | | | |
| | HuGene_st | | | | | |
| UGT1A3 | 116025-HuGene_st:6488-HuGene_st:881135- | 1.32E−11 | 2.07 | 4.56 | 85 | 77-90.9 |
| | HuGene_st:230953_at:221304_at:221305_s_at:511516- | | | | | |
| | HuGene_st:594963-HuGene_st:396121- | | | | | |
| | HuGene_st:123777-HuGene_st:1016481- | | | | | |
| | HuGene_st:204532_x_at:683377-HuGene_st:1055169- | | | | | |
| | HuGene_st:1035103-HuGene_st:1088102- | | | | | |
| | HuGene_st:207126_x_at:42874- | | | | | |
| | HuGene_st:215125_s_at:206094_x_at:208596_s_at:97211- | | | | | |
| | HuGene_st:1009861-HuGene_st:603368- | | | | | |
| | HuGene_st:232654_s_at:625897-HuGene_st:998604- | | | | | |
| | HuGene_st | | | | | |
| UGT1A9 | 116025-HuGene_st:6488-HuGene_st:881135- | 1.32E−11 | 2.0667 | 4.56 | 84.9 | 77-90.8 |
| | HuGene_st:230953_at:221304_at:221305_s_at:511516- | | | | | |
| | HuGene_st:594963-HuGene_st:396121- | | | | | |
| | HuGene_st:123777-HuGene_st:1016481- | | | | | |
| | HuGene_st:204532_x_at:683377-HuGene_st:1055169- | | | | | |
| | HuGene_st:1035103-HuGene_st:1088102- | | | | | |
| | HuGene_st:207126_x_at:42874- | | | | | |
| | HuGene_st:215125_s_at:206094_x_at:208596_s_at:97211- | | | | | |
| | HuGene_st:1009861-HuGene_st:603368- | | | | | |
| | HuGene_st:232654_s_at:625897-HuGene_st:998604- | | | | | |
| | HuGene_st | | | | | |
| PADI2 | 391522-HuGene_st:918392-HuGene_st:10163- | 1.46E−11 | 2.0234 | 3.98 | 84.4 | 76.4-90.5 |
| | HuGene_st:399093-HuGene_st:704572- | | | | | |
| | HuGene_st:1554385_a_at:366338-HuGene_st:440876- | | | | | |
| | HuGene_st:1075540-HuGene_st:963259- | | | | | |
| | HuGene_st:994995-HuGene_st:315189- | | | | | |
| | HuGene_st:272079-HuGene_st:243230- | | | | | |
| | HuGene_st:96325-HuGene_st:793116- | | | | | |
| | HuGene_st:362093-HuGene_st:390381- | | | | | |
| | HuGene_st:690679-HuGene_st:209791_at:173109- | | | | | |
| | HuGene_st:339627-HuGene_st:1554384_at | | | | | |
| CD177 | 993284-HuGene_st:444696-HuGene_st:79609- | 1.49E−11 | 2.0611 | 5.77 | 84.9 | 76.9-90.8 |
| | HuGene_st:969709-HuGene_st:369998- | | | | | |
| | HuGene_st:219669_at:572266-HuGene_st:536281- | | | | | |
| | HuGene_st:90319-HuGene_st:699790- | | | | | |
| | HuGene_st:531312-HuGene_st:707358- | | | | | |
| | HuGene_st:180891-HuGene_st:426820- | | | | | |
| | HuGene_st:1006900-HuGene_st:638144- | | | | | |
| | HuGene_st:232715-HuGene_st:134551- | | | | | |
| | HuGene_st:609683-HuGene_st | | | | | |
| FHL1 | 72610-HuGene_st:539308-HuGene_st:951657- | 1.63E−11 | 2.0432 | 3.25 | 84.7 | 76.7-90.6 |
| | HuGene_st:902973-HuGene_st:1001828- | | | | | |
| | HuGene_st:286356-HuGene_st:899294- | | | | | |
| | HuGene_st:25935- | | | | | |
| | HuGene_st:214505_s_at:210298_x_at:210299_s_at:602362- | | | | | |
| | HuGene_st:1055343-HuGene_st:201680- | | | | | |
| | HuGene_st:319870-HuGene_st:21197- | | | | | |
| | HuGene_st:36473-HuGene_st:689087- | | | | | |
| | HuGene_st:528922-HuGene_st:201539_s_at:1022628- | | | | | |
| | HuGene_st:201540_at:437953-HuGene_st:746514- | | | | | |
| | HuGene_st:829623-HuGene_st | | | | | |
| CEACAM1 | 209498_at:206576_s_at:211889_x_at:864113- | 2.66E−11 | 2.0448 | 2.85 | 84.7 | 76.7-90.6 |
| | HuGene_st:14136-HuGene_st:361816- | | | | | |
| | HuGene_st:959455-HuGene_st:428485- | | | | | |
| | HuGene_st:747162-HuGene_st:647202- | | | | | |
| | HuGene_st:472587-HuGene_st:344791- | | | | | |
| | HuGene_st:508072-HuGene_st:762013- | | | | | |
| | HuGene_st:546799-HuGene_st:213733- | | | | | |
| | HuGene_st:652484-HuGene_st:872278- | | | | | |
| | HuGene_st:211883_x_at:309256-HuGene_st:699246- | | | | | |
| | HuGene_st:698844-HuGene_st:677813- | | | | | |
| | HuGene_st:26592-HuGene_st:210610_at:276021- | | | | | |
| | HuGene_st:873164-HuGene_st | | | | | |
| FBLN1 | 546550-HuGene_st:562183- | 2.82E−11 | 2.0164 | 3.55 | 84.3 | 76.3-90.4 |
| | HuGene_st:202995_s_at:507438-HuGene_st:361582- | | | | | |
| | HuGene_st:134034-HuGene_st:804953- | | | | | |
| | HuGene_st:122878-HuGene_st:331523- | | | | | |
| | HuGene_st:1089938-HuGene_st:453797- | | | | | |
| | HuGene_st:106596-HuGene_st:862139- | | | | | |
| | HuGene_st:972691-HuGene_st:201787_at:687992- | | | | | |
| | HuGene_st:478818-HuGene_st:991293- | | | | | |
| | HuGene_st:328594-HuGene_st:844984- | | | | | |
| | HuGene_st:202994_s_at:675821- | | | | | |
| | HuGene_st:207835_at:55816-HuGene_st | | | | | |
| SEPP1 | 803943-HuGene_st:717194-HuGene_st:113867- | 2.83E−11 | 2.0327 | 4.15 | 84.5 | 76.6-90.6 |
| | HuGene_st:286524-HuGene_st:228585- | | | | | |
| | HuGene_st:824292-HuGene_st:754251- | | | | | |
| | HuGene_st:150171-HuGene_st:904197- | | | | | |
| | HuGene_st:116381-HuGene_st:267171- | | | | | |
| | HuGene_st:854108- | | | | | |
| | HuGene_st:201427_s_at:231669_at:327884- | | | | | |
| | HuGene_st:136636-HuGene_st:164967- | | | | | |
| | HuGene_st:1037558-HuGene_st:26213- | | | | | |
| | HuGene_st:456616-HuGene_st:1000147- | | | | | |
| | HuGene_st:748769-HuGene_st:658219- | | | | | |
| | HuGene_st:242519_at:229620_at:237475_x_at | | | | | |
| TBC1D9 | 237091_at:730535-HuGene_st:659317- | 3.89E−11 | 2.1454 | 1.54 | 85.8 | 78-91.5 |
| | HuGene_st:68121-HuGene_st:899166- | | | | | |
| | HuGene_st:785719-HuGene_st:138865- | | | | | |
| | HuGene_st:953028-HuGene_st:20928- | | | | | |
| | HuGene_st:969812-HuGene_st:591384- | | | | | |
| | HuGene_st:953516-HuGene_st:643103- | | | | | |
| | HuGene_st:821110-HuGene_st:988068- | | | | | |
| | HuGene_st:623144-HuGene_st:381615- | | | | | |
| | HuGene_st:708956-HuGene_st:212956_at | | | | | |
| CA12 | 89078-HuGene_st:363685-HuGene_st:814108- | 4.32E−11 | 2.0009 | 2.65 | 84.1 | 76-90.3 |
| | HuGene_st:125567-HuGene_st:226479- | | | | | |
| | HuGene_st:495486-HuGene_st:692924- | | | | | |
| | HuGene_st:203963_at:65565-HuGene_st:963408- | | | | | |
| | HuGene_st:1017708- | | | | | |
| | HuGene_st:210735_s_at:1009602-HuGene_st:875965- | | | | | |
| | HuGene_st:215867_x_at:749017-HuGene_st:638145- | | | | | |
| | HuGene_st:204508_s_at:214164_x_at:1049350- | | | | | |
| | HuGene_st:189118-HuGene_st:698714- | | | | | |
| | HuGene_st:1013062-HuGene_st:280470- | | | | | |
| | HuGene_st:439438-HuGene_st:1049334- | | | | | |
| | HuGene_st:204509_at:233388_at | | | | | |
| DMD | 945600-HuGene_st:12596-HuGene_st:434657- | 5.01E−11 | 2.0152 | 4.74 | 84.3 | 76.3-90.3 |
| | HuGene_st:676962-HuGene_st:1020969- | | | | | |
| | HuGene_st:170680-HuGene_st:855670- | | | | | |
| | HuGene_st:909848-HuGene_st:134644- | | | | | |
| | HuGene_st:887814-HuGene_st:649184- | | | | | |
| | HuGene_st:110990-HuGene_st:914217- | | | | | |
| | HuGene_st:961622-HuGene_st:514011- | | | | | |
| | HuGene_st:842792-HuGene_st:445483- | | | | | |
| | HuGene_st:208086_s_at:987362-HuGene_st:997308- | | | | | |
| | HuGene_st | | | | | |
| SMPDL3A | 734740-HuGene_st:406928-HuGene_st:871227- | 5.84E−11 | 1.941 | 3.14 | 83.4 | 75.2-89.7 |
| | HuGene_st:30922-HuGene_st:771860- | | | | | |
| | HuGene_st:376657-HuGene_st:539013- | | | | | |
| | HuGene_st:900092-HuGene_st:253658- | | | | | |
| | HuGene_st:696711-HuGene_st:284449- | | | | | |
| | HuGene_st:324159-HuGene_st:70962- | | | | | |
| | HuGene_st:161952-HuGene_st:873946- | | | | | |
| | HuGene_st:588668-HuGene_st:681531- | | | | | |
| | HuGene_st:195216-HuGene_st:213624_at:471149- | | | | | |
| | HuGene_st:1562512_at | | | | | |
| HSPB6 | 438264-HuGene_st:771601-HuGene_st:17884- | 6.16E−11 | 1.9492 | 6.88 | 83.5 | 75.4-89.8 |
| | HuGene_st:587849-HuGene_st:923983- | | | | | |
| | HuGene_st:214767_s_at:226304_at:763505- | | | | | |
| | HuGene_st:835176-HuGene_st:868647- | | | | | |
| | HuGene_st:511313-HuGene_st:848921-HuGene_st | | | | | |
| AKR1B10 | 1017995-HuGene_st:244038-HuGene_st:943992- | 7.91E−11 | 1.9484 | 5.17 | 83.5 | 75.3-89.7 |
| | HuGene_st:690334-HuGene_st:486646- | | | | | |
| | HuGene_st:234015-HuGene_st:599681- | | | | | |
| | HuGene_st:206561_s_at:347314-HuGene_st:827689- | | | | | |
| | HuGene_st:212880-HuGene_st:516961- | | | | | |
| | HuGene_st:638468-HuGene_st | | | | | |
| UGDH | 961600-HuGene_st:1046173-HuGene_st:509128- | 8.72E−11 | 1.9628 | 2.4 | 83.7 | 75.5-89.9 |
| | HuGene_st:428595-HuGene_st:363704- | | | | | |
| | HuGene_st:519385-HuGene_st:319601- | | | | | |
| | HuGene_st:653273-HuGene_st:121518- | | | | | |
| | HuGene_st:786461-HuGene_st:470924- | | | | | |
| | HuGene_st:530032-HuGene_st:810299- | | | | | |
| | HuGene_st:954191-HuGene_st:203343_at:855306- | | | | | |
| | HuGene_st:1075627-HuGene_st:184297- | | | | | |
| | HuGene_st:775350-HuGene_st:170400- | | | | | |
| | HuGene_st:374055-HuGene_st:186342-HuGene_st | | | | | |
| FABP1 | 825235-HuGene_st:208491-HuGene_st:626063- | 9.02E−11 | 1.968 | 6.54 | 83.7 | 75.6-89.9 |
| | HuGene_st:625600-HuGene_st:909598- | | | | | |
| | HuGene_st:350768-HuGene_st:760377- | | | | | |
| | HuGene_st:52477-HuGene_st:421055- | | | | | |
| | HuGene_st:205892_s_at:231693_at:699932- | | | | | |
| | HuGene_st:561606:HuGene_st:791463- | | | | | |
| | HuGene_st:495764-HuGene_st:703121- | | | | | |
| | HuGene_st:1025066-HuGene_st:392859- | | | | | |
| | HuGene_st:902525-HuGene_st:81171- | | | | | |
| | HuGene_st:1073289-HuGene_st:219434- | | | | | |
| | HuGene_st:735424-HuGene_st:297976-HuGene_st | | | | | |
| P2RY14 | 528057-HuGene_st:780310-HuGene_st:235178- | 1.31E−10 | 1.9821 | 2 | 83.9 | 75.8-90 |
| | HuGene_st:352954-HuGene_st:699489- | | | | | |
| | HuGene_st:38001-HuGene_st:637791- | | | | | |
| | HuGene_st:25606-HuGene_st:40647- | | | | | |
| | HuGene_st:896487-HuGene_st:672149- | | | | | |
| | HuGene_st:863820-HuGene_st:352427- | | | | | |
| | HuGene_st:632821-HuGene_st:116148- | | | | | |
| | HuGene_st:561792-HuGene_st:840910- | | | | | |
| | HuGene_st:296420-HuGene_st:974643- | | | | | |
| | HuGene_st:206637_at | | | | | |
| TTRAP | 369720-HuGene_st:914515-HuGene_st:43073- | 1.59E−10 | 1.9196 | 2.2 | 83.1 | 74.9-89.4 |
| | HuGene_st:85920-HuGene_st:278231- | | | | | |
| | HuGene_st:345627-HuGene_st:358211- | | | | | |
| | HuGene_st:331406-HuGene_st:748071- | | | | | |
| | HuGene_st:840774-HuGene_st:384150- | | | | | |
| | HuGene_st:528293-HuGene_st:202266_at:784674- | | | | | |
| | HuGene_st:370526-HuGene_st:1031392- | | | | | |
| | HuGene_st:258400-HuGene_st:1084398- | | | | | |
| | HuGene_st:223012-HuGene_st:1033688- | | | | | |
| | HuGene_st:232744-HuGene_st | | | | | |
| MEP1A | 854525-HuGene_st:233471-HuGene_st:521357- | 2.07E−10 | 1.9887 | 3.17 | 84 | 76-90.1 |
| | HuGene_st:398055-HuGene_st:479853- | | | | | |
| | HuGene_st:686103-HuGene_st:711452- | | | | | |
| | HuGene_st:984096-HuGene_st:1083396- | | | | | |
| | HuGene_st:1058253-HuGene_st:951000- | | | | | |
| | HuGene_st:984505-HuGene_st:390982- | | | | | |
| | HuGene_st:289221-HuGene_st:1044210- | | | | | |
| | HuGene_st:700879-HuGene_st:368627- | | | | | |
| | HuGene_st:1081805-HuGene_st:206000_at:54151- | | | | | |
| | HuGene_st:558849-HuGene_st:200136- | | | | | |
| | HuGene_st:519266-HuGene_st | | | | | |
| MYH11 | 628303-HuGene_st:20510-HuGene_st:77517- | 2.15E−10 | 1.8602 | 5.35 | 82.4 | 74-88.8 |
| | HuGene_st:847369-HuGene_st:396579- | | | | | |
| | HuGene_st:751338-HuGene_st:56832- | | | | | |
| | HuGene_st:227843_at:643005-HuGene_st:410559- | | | | | |
| | HuGene_st:618203-HuGene_st:64921- | | | | | |
| | HuGene_st:1083141-HuGene_st:724450- | | | | | |
| | HuGene_st:755245-HuGene_st:947574- | | | | | |
| | HuGene_st:201496_x_at:482335-HuGene_st:456670- | | | | | |
| | HuGene_st:974777-HuGene_st:86441- | | | | | |
| | HuGene_st:1066402-HuGene_st:289331- | | | | | |
| | HuGene_st:888083- | | | | | |
| | HuGene_st:201495_x_at:201497_x_at:207961_x_at:1568760_at: | | | | | |
| | 239307_at | | | | | |
| MAOA | 234534_at:353863-HuGene_st:1038534- | 2.49E−10 | 2.0268 | 2.18 | 84.5 | 76.5-90.5 |
| | HuGene_st:732300- | | | | | |
| | HuGene_st:204389_at:204388_s_at:211670- | | | | | |
| | HuGene_st:360481-HuGene_st:151427- | | | | | |
| | HuGene_st:347487-HuGene_st:791116- | | | | | |
| | HuGene_st:212741_at:1003275-HuGene_st:24543- | | | | | |
| | HuGene_st:126920-HuGene_st:500217- | | | | | |
| | HuGene_st:434659-HuGene_st:73861- | | | | | |
| | HuGene_st:382884-HuGene_st:675946- | | | | | |
| | HuGene_st:108162-HuGene_st:119160-HuGene_st | | | | | |
| CLDN8 | 1018006-HuGene_st:190634-HuGene_st:590280- | 2.92E−10 | 1.8632 | 12.96 | 82.4 | 74-88.9 |
| | HuGene_st:186468-HuGene_st:954438- | | | | | |
| | HuGene_st:428391-HuGene_st:480543- | | | | | |
| | HuGene_st:944337-HuGene_st:179725- | | | | | |
| | HuGene_st:508584-HuGene_st:1009114- | | | | | |
| | HuGene_st:948216-HuGene_st:658285- | | | | | |
| | HuGene_st:1022600-HuGene_st:737498- | | | | | |
| | HuGene_st:470015-HuGene_st:103315- | | | | | |
| | HuGene_st:699348-HuGene_st:89877- | | | | | |
| | HuGene_st:56937-HuGene_st:862663- | | | | | |
| | HuGene_st:504945-HuGene_st:214598_at | | | | | |
| FUCA1 | 295335-HuGene_st:550769-HuGene_st:674252- | 4.24E−10 | 1.8845 | 2.03 | 82.7 | 74.4-89.1 |
| | HuGene_st:343732-HuGene_st:10170- | | | | | |
| | HuGene_st:1012064-HuGene_st:296930- | | | | | |
| | HuGene_st:179394-HuGene_st:587765- | | | | | |
| | HuGene_st:229137_at:142430-HuGene_st:801038- | | | | | |
| | HuGene_st:447607-HuGene_st:66284- | | | | | |
| | HuGene_st:937530-HuGene_st:884152- | | | | | |
| | HuGene_st:674608-HuGene_st:202838_at:106313- | | | | | |
| | HuGene_st:602308-HuGene_st:887004- | | | | | |
| | HuGene_st:206251-HuGene_st:357516-HuGene_st | | | | | |
| FXYD6 | 138547-HuGene_st:814211-HuGene_st:185591- | 6.39E−10 | 1.8087 | 2.92 | 81.7 | 73.3-88.3 |
| | HuGene_st:992039-HuGene_st:549291- | | | | | |
| | HuGene_st:921510-HuGene_st:163300- | | | | | |
| | HuGene_st:4792-HuGene_st:369594- | | | | | |
| | HuGene_st:209348-HuGene_st:1028937- | | | | | |
| | HuGene_st:77232-HuGene_st:529384- | | | | | |
| | HuGene_st:217897_at:877189-HuGene_st:973770- | | | | | |
| | HuGene_st:1054980-HuGene_st:240323_at:466048- | | | | | |
| | HuGene_st | | | | | |
| ARL14 | 220468_at:471121-HuGene_st:690873- | 7.01E−10 | 1.8255 | 3.06 | 81.9 | 73.5-88.4 |
| | HuGene_st:468858-HuGene_st:750196- | | | | | |
| | HuGene_st:62407-HuGene_st:159605- | | | | | |
| | HuGene_st:979866-HuGene_st:1032832- | | | | | |
| | HuGene_st:446532-HuGene_st:861783- | | | | | |
| | HuGene_st:116203-HuGene_st:991610- | | | | | |
| | HuGene_st:515168-HuGene_st:1073679- | | | | | |
| | HuGene_st:439428-HuGene_st:976681- | | | | | |
| | HuGene_st:452044-HuGene_st:197209- | | | | | |
| | HuGene_st:12441-HuGene_st | | | | | |
| SLC20A1 | 335219-HuGene_st:230038-HuGene_st:1078924- | 7.65E−10 | 1.7854 | 4.04 | 81.4 | 72.9-88.1 |
| | HuGene_st:520074-HuGene_st:769870- | | | | | |
| | HuGene_st:175687-HuGene_st:541149- | | | | | |
| | HuGene_st:973433-HuGene_st:643658- | | | | | |
| | HuGene_st:220784-HuGene_st:175599- | | | | | |
| | HuGene_st:482373-HuGene_st:1003908- | | | | | |
| | HuGene_st:337454-HuGene_st:655800- | | | | | |
| | HuGene_st:321368-HuGene_st:230494_at:178021- | | | | | |
| | HuGene_st:201920_at:448729-HuGene_st:896684- | | | | | |
| | HuGene_st:363789-HuGene_st:130920- | | | | | |
| | HuGene_st:786196-HuGene_st | | | | | |
| MAB21L2 | 338297-HuGene_st:994188-HuGene_st:109548- | 1.05E−09 | 1.7739 | 5.2 | 81.2 | 72.7-87.9 |
| | HuGene_st:395419-HuGene_st:922092- | | | | | |
| | HuGene_st:737843-HuGene_st:6731- | | | | | |
| | HuGene_st:708698-HuGene_st:352943- | | | | | |
| | HuGene_st:485256-HuGene_st:1023066- | | | | | |
| | HuGene_st:825747-HuGene_st:210303_at:309088- | | | | | |
| | HuGene_st:1031483-HuGene_st:441727- | | | | | |
| | HuGene_st:751700-HuGene_st:974270- | | | | | |
| | HuGene_st:210302_s_at:254681-HuGene_st:982880- | | | | | |
| | HuGene_st | | | | | |
| TSPAN1 | 833585-HuGene_st:209114_at:664359- | 1.16E−09 | 1.7875 | 2.16 | 81.4 | 73-88.1 |
| | HuGene_st:307816-HuGene_st:111939- | | | | | |
| | HuGene_st:39185-HuGene_st:496300- | | | | | |
| | HuGene_st:1038310-HuGene_st:401084- | | | | | |
| | HuGene_st:632040-HuGene_st:1076526- | | | | | |
| | HuGene_st:488552-HuGene_st:35199- | | | | | |
| | HuGene_st:611793-HuGene_st:1007567- | | | | | |
| | HuGene_st:305523-HuGene_st:1033895- | | | | | |
| | HuGene_st:145680-HuGene_st:812712- | | | | | |
| | HuGene_st:929225-HuGene_st:641156- | | | | | |
| | HuGene_st:116814-HuGene_st | | | | | |
| KLF4 | 310345-HuGene_st:421025-HuGene_st:853068- | 1.19E−09 | 1.8535 | 2.27 | 82.3 | 74-88.8 |
| | HuGene_st:1040228-HuGene_st:1040166- | | | | | |
| | HuGene_st:549197-HuGene_st:257340- | | | | | |
| | HuGene_st:95161-HuGene_st:166058- | | | | | |
| | HuGene_st:749339-HuGene_st:212619- | | | | | |
| | HuGene_st:220266_s_at:1078952-HuGene_st:289946- | | | | | |
| | HuGene_st:824845-HuGene_st:965293- | | | | | |
| | HuGene_st:467978-HuGene_st:287320- | | | | | |
| | HuGene_st:334405-HuGene_st:234619- | | | | | |
| | HuGene_st:221841_s_at:145954-HuGene_st:733888- | | | | | |
| | HuGene_st | | | | | |
| CRYAB | 581894-HuGene_st:42986-HuGene_st:626903- | 1.47E−09 | 1.768 | 3.91 | 81.2 | 72.7-87.9 |
| | HuGene_st:612490-HuGene_st:1017597- | | | | | |
| | HuGene_st:473983-HuGene_st:380047- | | | | | |
| | HuGene_st:25982-HuGene_st:298978- | | | | | |
| | HuGene_st:485492-HuGene_st:413012- | | | | | |
| | HuGene_st:944836-HuGene_st:209283_at:615857- | | | | | |
| | HuGene_st:303036-HuGene_st:592729- | | | | | |
| | HuGene_st:349645-HuGene_st:226566- | | | | | |
| | HuGene_st:215779-HuGene_st:702453- | | | | | |
| | HuGene_st:95542-HuGene_st:697816-HuGene_st | | | | | |
| C15orf48 | 661133-HuGene_st:240581-HuGene_st:302164- | 1.54E−09 | 1.8166 | 3.07 | 81.8 | 73.5-88.4 |
| | HuGene_st:262469-HuGene_st:512593- | | | | | |
| | HuGene_st:122711-HuGene_st:269066- | | | | | |
| | HuGene_st:246950-HuGene_st:483493- | | | | | |
| | HuGene_st:352750-HuGene_st:645087- | | | | | |
| | HuGene_st:775549-HuGene_st:223484_at:521885- | | | | | |
| | HuGene_st:1004430-HuGene_st:885617- | | | | | |
| | HuGene_st:166185-HuGene_st:752633- | | | | | |
| | HuGene_st:1056973-HuGene_st:60479-HuGene_st | | | | | |
| ACAT1 | 205412_at:877718-HuGene_st:697995- | 1.70E−09 | 1.8589 | 1.77 | 82.4 | 74-88.8 |
| | HuGene_st:244911-HuGene_st:705306- | | | | | |
| | HuGene_st:110401-HuGene_st:31594- | | | | | |
| | HuGene_st:19333-HuGene_st:499096- | | | | | |
| | HuGene_st:942019-HuGene_st:618952- | | | | | |
| | HuGene_st:933332-HuGene_st:715946- | | | | | |
| | HuGene_st:676239-HuGene_st:172044- | | | | | |
| | HuGene_st:828361-HuGene_st:321829- | | | | | |
| | HuGene_st:897946-HuGene_st:331003- | | | | | |
| | HuGene_st:1559239_s_at:814756-HuGene_st | | | | | |
| ANPEP | 42832-HuGene_st:567567-HuGene_st:992744- | 2.04E−09 | 1.7687 | 8.18 | 81.2 | 72.7-87.9 |
| | HuGene_st:524001-HuGene_st:229640- | | | | | |
| | HuGene_st:372330-HuGene_st:842255- | | | | | |
| | HuGene_st:42234-HuGene_st:640878- | | | | | |
| | HuGene_st:702098-HuGene_st:917110- | | | | | |
| | HuGene_st:25968-HuGene_st:850385- | | | | | |
| | HuGene_st:503143-HuGene_st:202888_s_at:429651- | | | | | |
| | HuGene_st:409574-HuGene_st:60717- | | | | | |
| | HuGene_st:998436-HuGene_st:618326- | | | | | |
| | HuGene_st:1067290-HuGene_st:333846-HuGene_st | | | | | |
| FGL2 | 884418-HuGene_st:249194-HuGene_st:463016- | 2.64E−09 | 1.8254 | 2.07 | 81.9 | 73.5-88.5 |
| | HuGene_st:194197-HuGene_st:789123- | | | | | |
| | HuGene_st:998752-HuGene_st:596140- | | | | | |
| | HuGene_st:896312-HuGene_st:762195- | | | | | |
| | HuGene_st:909927-HuGene_st:686836- | | | | | |
| | HuGene_st:381278-HuGene_st:463952- | | | | | |
| | HuGene_st:757331-HuGene_st:206261- | | | | | |
| | HuGene_st:706108-HuGene_st:202575- | | | | | |
| | HuGene_st:148017-HuGene_st:199906- | | | | | |
| | HuGene_st:707754-HuGene_st:204834_at | | | | | |
| PPID | 204185_x_at:204186_s_at | 3.34E−09 | 1.8025 | 1.73 | 81.6 | 73.2-88.2 |
| ITM2C | 83688-HuGene_st:398134-HuGene_st:972817- | 3.87E−09 | 1.7063 | 2.22 | 80.3 | 71.7-87.2 |
| | HuGene_st:698719-HuGene_st:369206- | | | | | |
| | HuGene_st:377066-HuGene_st:23436- | | | | | |
| | HuGene_st:593996-HuGene_st:987556- | | | | | |
| | HuGene_st:221004_s_at:465490-HuGene_st:776424- | | | | | |
| | HuGene_st:1052550-HuGene_st:344231- | | | | | |
| | HuGene_st:1039973-HuGene_st:127312- | | | | | |
| | HuGene_st:577346-HuGene_st:394326- | | | | | |
| | HuGene_st:848902-HuGene_st | | | | | |
| SST | 477599-HuGene_st:1101191-HuGene_st:474819- | 4.16E−09 | 1.7627 | 6.14 | 81.1 | 72.6-87.8 |
| | HuGene_st:144116-HuGene_st:76078- | | | | | |
| | HuGene_st:698582-HuGene_st:600708- | | | | | |
| | HuGene_st:98570-HuGene_st:4754- | | | | | |
| | HuGene_st:675107-HuGene_st:213921_at:115911- | | | | | |
| | HuGene_st:235647-HuGene_st:131210- | | | | | |
| | HuGene_st:502176-HuGene_st:63243-HuGene_st | | | | | |
| PRIMA1 | 1084922-HuGene_st:1008125-HuGene_st:22062- | 4.74E−09 | 1.7053 | 2.75 | 80.3 | 71.7-87.1 |
| | HuGene_st:182482-HuGene_st:225291- | | | | | |
| | HuGene_st:566243-HuGene_st:50975- | | | | | |
| | HuGene_st:230087_at:787213-HuGene_st:661160- | | | | | |
| | HuGene_st:256004-HuGene_st:6276- | | | | | |
| | HuGene_st:44738-HuGene_st | | | | | |
| EFEMP1 | 201842_s_at:1073952- | 5.80E−09 | 1.8208 | 1.69 | 81.9 | 73.4-88.4 |
| | HuGene_st:228421_s_at:520435-HuGene_st:551581- | | | | | |
| | HuGene_st:909695-HuGene_st:539520- | | | | | |
| | HuGene_st:719105-HuGene_st:1046788- | | | | | |
| | HuGene_st:728978-HuGene_st:956186- | | | | | |
| | HuGene_st:89258-HuGene_st:155486- | | | | | |
| | HuGene_st:986491-HuGene_st:207603- | | | | | |
| | HuGene_st:243191-HuGene_st:842290- | | | | | |
| | HuGene_st:757697-HuGene_st:784694- | | | | | |
| | HuGene_st:132382-HuGene_st | | | | | |
| ADAMTS1 | 432578-HuGene_st:139364-HuGene_st:423315- | 6.42E−09 | 1.7467 | 2.37 | 80.9 | 72.4-87.6 |
| | HuGene_st:578666-HuGene_st:419311- | | | | | |
| | HuGene_st:928538-HuGene_st:344696- | | | | | |
| | HuGene_st:617885-HuGene_st:136304- | | | | | |
| | HuGene_st:75724-HuGene_st:412640- | | | | | |
| | HuGene_st:368117-HuGene_st:97806- | | | | | |
| | HuGene_st:641285-HuGene_st:222486_s_at:711563- | | | | | |
| | HuGene_st:1006281-HuGene_st:222162_s_at:32418- | | | | | |
| | HuGene_st | | | | | |
| AKAP12 | 212419-HuGene_st:231067_s_at:379659- | 7.41E−09 | 1.744 | 2.83 | 80.8 | 72.2-87.5 |
| | HuGene_st:1010338-HuGene_st:1075094- | | | | | |
| | HuGene_st:42401-HuGene_st:522584- | | | | | |
| | HuGene_st:480972-HuGene_st:948623- | | | | | |
| | HuGene_st:701945-HuGene_st:276784- | | | | | |
| | HuGene_st:64858-HuGene_st:210517_s_at:874382- | | | | | |
| | HuGene_st:909976-HuGene_st:182037- | | | | | |
| | HuGene_st:417182-HuGene_st:722881-HuGene_st | | | | | |
| COL14A1 | 216866_s_at:309808-HuGene_st:532905- | 7.60E−09 | 1.7817 | 1.67 | 81.3 | 72.9-88 |
| | HuGene_st:938881-HuGene_st:184937- | | | | | |
| | HuGene_st:332362-HuGene_st:431855- | | | | | |
| | HuGene_st:295715-HuGene_st:813208- | | | | | |
| | HuGene_st:216865_at:426456- | | | | | |
| | HuGene_st:212865_s_at:513900-HuGene_st:1054393- | | | | | |
| | HuGene_st:947013-HuGene_st:966609- | | | | | |
| | HuGene_st:929330-HuGene_st:283165- | | | | | |
| | HuGene_st:67940-HuGene_st:655615- | | | | | |
| | HuGene_st:236228-HuGene_st | | | | | |
| UGT2A3 | 149647-HuGene_st:860083-HuGene_st:922544- | 8.64E−09 | 1.7414 | 4.89 | 80.8 | 72.2-87.5 |
| | HuGene_st:244206-HuGene_st:503323- | | | | | |
| | HuGene_st:353576-HuGene_st:603619- | | | | | |
| | HuGene_st:787458-HuGene_st:219796- | | | | | |
| | HuGene_st:333564-HuGene_st:257402- | | | | | |
| | HuGene_st:366699-HuGene_st:461685- | | | | | |
| | HuGene_st:891681-HuGene_st:644952- | | | | | |
| | HuGene_st:621618-HuGene_st:737617- | | | | | |
| | HuGene_st:88682-HuGene_st:529761- | | | | | |
| | HuGene_st:895203-HuGene_st:658594- | | | | | |
| | HuGene_st:455115-HuGene_st | | | | | |
| PTGIS | 162553-HuGene_st:277727-HuGene_st:426160- | 9.15E−09 | 1.7213 | 3.98 | 80.5 | 72-87.3 |
| | HuGene_st:49754-HuGene_st:491989- | | | | | |
| | HuGene_st:846155-HuGene_st:214744- | | | | | |
| | HuGene_st:81045-HuGene_st:757700- | | | | | |
| | HuGene_st:713439-HuGene_st:765856- | | | | | |
| | HuGene_st:211892_s_at:424645-HuGene_st:805861- | | | | | |
| | HuGene_st:16678-HuGene_st:49009- | | | | | |
| | HuGene_st:210702_s_at:33677-HuGene_st:757535- | | | | | |
| | HuGene_st:1065183-HuGene_st:949066-HuGene_st | | | | | |
| BEST2 | 242542-HuGene_st:368041-HuGene_st:316182- | 9.19E−09 | 1.7181 | 3.63 | 80.5 | 71.9-87.3 |
| | HuGene_st:417689-HuGene_st:953359- | | | | | |
| | HuGene_st:196857-HuGene_st:2545- | | | | | |
| | HuGene_st:207432_at:371039-HuGene_st:105864- | | | | | |
| | HuGene_st:946835-HuGene_st:371107- | | | | | |
| | HuGene_st:170027-HuGene_st:238631- | | | | | |
| | HuGene_st:515622-HuGene_st:886418- | | | | | |
| | HuGene_st:396311-HuGene_st:735639- | | | | | |
| | HuGene_st:947978-HuGene_st:882286- | | | | | |
| | HuGene_st:894968-HuGene_st | | | | | |
| MS4A4A | 651445-HuGene_st:308636-HuGene_st:174209- | 9.59E−09 | 1.8179 | 1.55 | 81.8 | 73.4-88.4 |
| | HuGene_st:486453-HuGene_st:219607_s_at:1041327- | | | | | |
| | HuGene_st:84116-HuGene_st:161192- | | | | | |
| | HuGene_st:491070-HuGene_st:1555728_a_at:142937- | | | | | |
| | HuGene_st:581150-HuGene_st:579786- | | | | | |
| | HuGene_st:588041-HuGene_st:224357_s_at:886807- | | | | | |
| | HuGene_st:122093-HuGene_st | | | | | |
| SGCE | 976641-HuGene_st:204688_at:241763- | 1.02E−08 | 1.7444 | 2.03 | 80.8 | 72.3-87.6 |
| | HuGene_st:83484-HuGene_st:281261- | | | | | |
| | HuGene_st:324304-HuGene_st:683152- | | | | | |
| | HuGene_st:850448-HuGene_st:254097- | | | | | |
| | HuGene_st:992961-HuGene_st:69099- | | | | | |
| | HuGene_st:781384-HuGene_st:899200- | | | | | |
| | HuGene_st:974761-HuGene_st:417302- | | | | | |
| | HuGene_st:211350-HuGene_st:198065-HuGene_st | | | | | |
| TPSB2 | 207134_x_at:207741_x_at | 1.24E−08 | 1.3708 | 1.87 | 75.3 | 66.3-83 |
| COL6A2 | 919359-HuGene_st:40510-HuGene_st:375216- | 1.30E−08 | 1.6771 | 2.08 | 79.9 | 71.2-86.8 |
| | HuGene_st:1086501-HuGene_st:160736- | | | | | |
| | HuGene_st:602554-HuGene_st:148827- | | | | | |
| | HuGene_st:794188-HuGene_st:213290_at:536399- | | | | | |
| | HuGene_st:501950-HuGene_st:404866- | | | | | |
| | HuGene_st:209156_s_at:813295-HuGene_st | | | | | |
| PRKACB | 202741_at:795011-HuGene_st:360410- | 1.30E−08 | 1.7234 | 2.18 | 80.6 | 72-87.3 |
| | HuGene_st:970814-HuGene_st:70528- | | | | | |
| | HuGene_st:1042799-HuGene_st:202742_s_at:964845- | | | | | |
| | HuGene_st:563484-HuGene_st:142353- | | | | | |
| | HuGene_st:597859-HuGene_st:660092- | | | | | |
| | HuGene_st:948901-HuGene_st:301751- | | | | | |
| | HuGene_st:294241-HuGene_st:694446- | | | | | |
| | HuGene_st:309819-HuGene_st:668357- | | | | | |
| | HuGene_st:589422-HuGene_st | | | | | |
| SPARCL1 | 246509-HuGene_st:300147-HuGene_st:525246- | 1.33E−08 | 1.634 | 2.85 | 79.3 | 70.5-86.3 |
| | HuGene_st:380181-HuGene_st:257526- | | | | | |
| | HuGene_st:585652-HuGene_st:733855- | | | | | |
| | HuGene_st:517768-HuGene_st:853991- | | | | | |
| | HuGene_st:854537-HuGene_st:1023002- | | | | | |
| | HuGene_st:55936-HuGene_st:371227- | | | | | |
| | HuGene_st:782475-HuGene_st:670571- | | | | | |
| | HuGene_st:1026998-HuGene_st:894310- | | | | | |
| | HuGene_st:948826-HuGene_st:302303- | | | | | |
| | HuGene_st:151789-HuGene_st:905519- | | | | | |
| | HuGene_st:200795_at:602848-HuGene_st | | | | | |
| PBLD | 1003993-HuGene_st:308979-HuGene_st:622498- | 1.38E−08 | 1.6983 | 2.74 | 80.2 | 71.5-87 |
| | HuGene_st:840594-HuGene_st:84006- | | | | | |
| | HuGene_st:636001-HuGene_st:529256- | | | | | |
| | HuGene_st:651369-HuGene_st:379972- | | | | | |
| | HuGene_st:492235-HuGene_st:584905- | | | | | |
| | HuGene_st:222771-HuGene_st:383791- | | | | | |
| | HuGene_st:1016249-HuGene_st:819013- | | | | | |
| | HuGene_st:1555175_a_at:115400-HuGene_st:969544- | | | | | |
| | HuGene_st:949581-HuGene_st:897360- | | | | | |
| | HuGene_st:219543_at:548506-HuGene_st | | | | | |
| PDK4 | 806272-HuGene_st:44901-HuGene_st:272445- | 1.45E−08 | 1.7059 | 3.02 | 80.3 | 71.8-87.1 |
| | HuGene_st:650209-HuGene_st:796256- | | | | | |
| | HuGene_st:342230-HuGene_st:648024- | | | | | |
| | HuGene_st:205960_at:25700-HuGene_st:152414- | | | | | |
| | HuGene_st:360382-HuGene_st:471160- | | | | | |
| | HuGene_st:219862-HuGene_st:584279- | | | | | |
| | HuGene_st:225207_at:498670-HuGene_st:405050- | | | | | |
| | HuGene_st:546964-HuGene_st:735039- | | | | | |
| | HuGene_st:1562321_at:348010-HuGene_st:1073661- | | | | | |
| | HuGene_st:578954-HuGene_st:966364- | | | | | |
| | HuGene_st:115766-HuGene_st | | | | | |
| DES | 680953-HuGene_st:710186-HuGene_st:224168- | 1.47E−08 | 1.7257 | 6.66 | 80.6 | 72-87.4 |
| | HuGene_st:720121-HuGene_st:632911- | | | | | |
| | HuGene_st:14146-HuGene_st:472740- | | | | | |
| | HuGene_st:452392-HuGene_st:70983- | | | | | |
| | HuGene_st:626407-HuGene_st:330060- | | | | | |
| | HuGene_st:678707-HuGene_st:202222_s_at:830447- | | | | | |
| | HuGene_st:466888-HuGene_st:832644- | | | | | |
| | HuGene_st:190691-HuGene_st:1043063- | | | | | |
| | HuGene_st:476251-HuGene_st:856127- | | | | | |
| | HuGene_st:680072-HuGene_st:1053701- | | | | | |
| | HuGene_st:490364-HuGene_st:954860- | | | | | |
| | HuGene_st:7755-HuGene_st:856708- | | | | | |
| | HuGene_st:272796-HuGene_st:377421- | | | | | |
| | HuGene_st:214027_x_at:458352-HuGene_st:104906- | | | | | |
| | HuGene_st:276614-HuGene_st | | | | | |
| MMP28 | 578497-HuGene_st:31358-HuGene_st:283053- | 1.80E−08 | 1.663 | 2.47 | 79.7 | 71.1-86.7 |
| | HuGene_st:152786-HuGene_st:519390- | | | | | |
| | HuGene_st:239273_s_at:599795-HuGene_st:800954- | | | | | |
| | HuGene_st:219909_at:190189- | | | | | |
| | HuGene_st:222937_s_at:1084226-HuGene_st:315451- | | | | | |
| | HuGene_st:100436-HuGene_st:794275- | | | | | |
| | HuGene_st:256212-HuGene_st:8583- | | | | | |
| | HuGene_st:987411- | | | | | |
| | HuGene_st:239272_at:224207_x_at:212418- | | | | | |
| | HuGene_st:319017-HuGene_st:604727-HuGene_st | | | | | |
| CYBRD1 | 580256-HuGene_st:931026-HuGene_st:412808- | 1.84E−08 | 1.6939 | 2.7 | 80.1 | 71.6-87 |
| | HuGene_st:138769-HuGene_st:199471- | | | | | |
| | HuGene_st:675528-HuGene_st:217889_s_at:570877- | | | | | |
| | HuGene_st:438204-HuGene_st:836676- | | | | | |
| | HuGene_st:251906-HuGene_st:71842- | | | | | |
| | HuGene_st:140118-HuGene_st:266609- | | | | | |
| | HuGene_st:132102-HuGene_st:774519- | | | | | |
| | HuGene_st:1068423-HuGene_st:995512- | | | | | |
| | HuGene_st:298248-HuGene_st:1099283- | | | | | |
| | HuGene_st:222189-HuGene_st:222453_at:232459_at | | | | | |
| IL6ST | 1054392-HuGene_st:234474_x_at:953995- | 1.87E−08 | 1.7642 | 1.58 | 81.1 | 72.6-87.8 |
| | HuGene_st:234967_at:48974-HuGene_st:325364- | | | | | |
| | HuGene_st:32344-HuGene_st:821422- | | | | | |
| | HuGene_st:298043-HuGene_st:606460- | | | | | |
| | HuGene_st:1074930-HuGene_st:44790- | | | | | |
| | HuGene_st:204864_s_at:1059165-HuGene_st:288593- | | | | | |
| | HuGene_st:39514-HuGene_st:204863_s_at:175414- | | | | | |
| | HuGene_st:293958-HuGene_st:212196_at:148922- | | | | | |
| | HuGene_st:211000_s_at:648367-HuGene_st:240067- | | | | | |
| | HuGene_st:212195_at:822552-HuGene_st:523608- | | | | | |
| | HuGene_st:474929-HuGene_st | | | | | |
| FABP4 | 547124-HuGene_st:96292-HuGene_st:545014- | 2.01E−08 | 1.6776 | 5.55 | 79.9 | 71.2-86.8 |
| | HuGene_st:22370-HuGene_st:138079- | | | | | |
| | HuGene_st:1064936-HuGene_st:804933- | | | | | |
| | HuGene_st:1087992-HuGene_st:20784- | | | | | |
| | HuGene_st:671128-HuGene_st:328798- | | | | | |
| | HuGene_st:594739-HuGene_st:1047009- | | | | | |
| | HuGene_st:825613-HuGene_st:637074- | | | | | |
| | HuGene_st:759940-HuGene_st:754024- | | | | | |
| | HuGene_st:88372-HuGene_st | | | | | |
| CALM1 | 196751-HuGene_st:1094382- | 2.12E−08 | 1.6819 | 1.72 | 80 | 71.3-86.9 |
| | HuGene_st:211985_s_at:445194- | | | | | |
| | HuGene_st:211984_at:213688_at:213710_s_at:444099- | | | | | |
| | HuGene_st:708994-HuGene_st:418220- | | | | | |
| | HuGene_st:241614_at:96889-HuGene_st:50211- | | | | | |
| | HuGene_st:200653_s_at:767359- | | | | | |
| | HuGene_st:200655_s_at:616208-HuGene_st:1019255- | | | | | |
| | HuGene_st:209563_x_at:715468-HuGene_st:18183- | | | | | |
| | HuGene_st:544779-HuGene_st:239705- | | | | | |
| | HuGene_st:1084911-HuGene_st:98189-HuGene_st | | | | | |
| TNS1 | 766751-HuGene_st:503077-HuGene_st:455229- | 2.21E−08 | 1.7412 | 2.01 | 80.8 | 72.3-87.5 |
| | HuGene_st:905238-HuGene_st:129966- | | | | | |
| | HuGene_st:750627-HuGene_st:989199- | | | | | |
| | HuGene_st:61598-HuGene_st:671515- | | | | | |
| | HuGene_st:129638-HuGene_st:1088728- | | | | | |
| | HuGene_st:693249-HuGene_st:418601- | | | | | |
| | HuGene_st:168879-HuGene_st:220476- | | | | | |
| | HuGene_st:23579-HuGene_st:731710- | | | | | |
| | HuGene_st:476750-HuGene_st:295235- | | | | | |
| | HuGene_st:218863_s_at:665955- | | | | | |
| | HuGene_st:221747_at:105442-HuGene_st:676236- | | | | | |
| | HuGene_st:124848-HuGene_st:474353- | | | | | |
| | HuGene_st:218864_at:925862-HuGene_st:279934- | | | | | |
| | HuGene_st:1008295-HuGene_st:221246_x_at:384388- | | | | | |
| | HuGene_st:522313-HuGene_st:754212- | | | | | |
| | HuGene_st:221748_s_at:420410-HuGene_st:250619- | | | | | |
| | HuGene_st | | | | | |
| EMP1 | 201324_at:311769-HuGene_st:201325_s_at:506469- | 2.59E−08 | 1.7156 | 1.95 | 80.4 | 71.9-87.3 |
| | HuGene_st:861706-HuGene_st:46080- | | | | | |
| | HuGene_st:236046-HuGene_st:548197- | | | | | |
| | HuGene_st:100442-HuGene_st:1096474- | | | | | |
| | HuGene_st:717809-HuGene_st:596930- | | | | | |
| | HuGene_st:468199-HuGene_st:751888- | | | | | |
| | HuGene_st:166375-HuGene_st:213895_at:871061- | | | | | |
| | HuGene_st:164793-HuGene_st:509462- | | | | | |
| | HuGene_st:182838-HuGene_st:1564796_at:446286- | | | | | |
| | HuGene_st | | | | | |
| RDX | 350620-HuGene_st:1005467-HuGene_st:284799- | 3.06E−08 | 1.7426 | 1.9 | 80.8 | 72.2-87.6 |
| | HuGene_st:78392-HuGene_st:923159- | | | | | |
| | HuGene_st:204969_s_at:935366-HuGene_st:603982- | | | | | |
| | HuGene_st:418137-HuGene_st:304555- | | | | | |
| | HuGene_st:174237-HuGene_st:140908- | | | | | |
| | HuGene_st:1034349-HuGene_st:212398_at:977130- | | | | | |
| | HuGene_st:6485-HuGene_st | | | | | |
| CFD | 396618-HuGene_st:672277-HuGene_st:290815 | −3.06E−08 | 1.6465 | 2.81 | 79.5 | 70.8-86.4 |
| | HuGene_st:304988-HuGene_st:580394- | | | | | |
| | HuGene_st:605542-HuGene_st:698498- | | | | | |
| | HuGene_st:580177-HuGene_st:633095- | | | | | |
| | HuGene_st:35339-HuGene_st:530155- | | | | | |
| | HuGene_st:584306-HuGene_st:205382_s_at:915940- | | | | | |
| | HuGene_st:859510-HuGene_st:978939- | | | | | |
| | HuGene_st:59414-HuGene_st:557142- | | | | | |
| | HuGene_st:104453-HuGene_st | | | | | |
| GPNMB | 201141_at:25823-HuGene_st:926046- | 3.11E−08 | 1.6924 | 1.99 | 80.1 | 71.5-87 |
| | HuGene_st:724736-HuGene_st:591503- | | | | | |
| | HuGene_st:660677-HuGene_st:701912- | | | | | |
| | HuGene_st:267933-HuGene_st:799615- | | | | | |
| | HuGene_st:569803-HuGene_st:979125- | | | | | |
| | HuGene_st:38037-HuGene_st:89805- | | | | | |
| | HuGene_st:335128-HuGene_st:131027- | | | | | |
| | HuGene_st:908272-HuGene_st:950837- | | | | | |
| | HuGene_st:39441-HuGene_st:1056618- | | | | | |
| | HuGene_st:860466-HuGene_st | | | | | |
| TPSAB1 | 350712-HuGene_st:134899-HuGene_st:122333- | 3.52E−08 | 1.7066 | 1.93 | 80.3 | 71.7-87.2 |
| | HuGene_st:1098415-HuGene_st:665622- | | | | | |
| | HuGene_st:701849-HuGene_st:535697- | | | | | |
| | HuGene_st:163189-HuGene_st:744603- | | | | | |
| | HuGene_st:153098- | | | | | |
| | HuGene_st:216485_s_at:216474_x_at:205683_x_at:416078- | | | | | |
| | HuGene_st:162760- | | | | | |
| | HuGene_st:217023_x_at:520284- | | | | | |
| | HuGene_st:215382_x_at:210084_x_at:567472- | | | | | |
| | HuGene_st:519234-HuGene_st:154829- | | | | | |
| | HuGene_st:247406-HuGene_st:331572- | | | | | |
| | HuGene_st:264147-HuGene_st:462114- | | | | | |
| | HuGene_st:880961-HuGene_st:705156-HuGene_st | | | | | |
| PPP1R14A | 374630-HuGene_st:551938-HuGene_st:783588- | 3.54E−08 | 1.6167 | 2.51 | 79.1 | 70.3-86.1 |
| | HuGene_st:44913-HuGene_st:966135- | | | | | |
| | HuGene_st:227006_at:5374729-HuGene_st:815370- | | | | | |
| | HuGene_st:1040980-HuGene_st:76662-HuGene_st | | | | | |
| LOC253012 | 873053-HuGene_st:945097-HuGene_st:896826- | 3.59E−08 | 1.6527 | 4.46 | 79.6 | 70.9-86.5 |
| | HuGene_st:363568-HuGene_st:162938- | | | | | |
| | HuGene_st:441915-HuGene_st:1070854- | | | | | |
| | HuGene_st:463072-HuGene_st:199784- | | | | | |
| | HuGene_st:304834-HuGene_st:381523- | | | | | |
| | HuGene_st:568837-HuGene_st:39629- | | | | | |
| | HuGene_st:1026726-HuGene_st:1014415- | | | | | |
| | HuGene_st:51876-HuGene_st:749900- | | | | | |
| | HuGene_st:804632-HuGene_st | | | | | |
| DMN | 207872-HuGene_st:407332-HuGene_st:828944- | 4.36E−08 | 1.6041 | 4.05 | 78.9 | 70-86 |
| | HuGene_st:469719-HuGene_st:166409- | | | | | |
| | HuGene_st:430636-HuGene_st:87850- | | | | | |
| | HuGene_st:1061724-HuGene_st:1040362- | | | | | |
| | HuGene_st:336607-HuGene_st:799683- | | | | | |
| | HuGene_st:504888-HuGene_st:212730_at:872910- | | | | | |
| | HuGene_st:861909-HuGene_st:406723- | | | | | |
| | HuGene_st:530704-HuGene_st:214304_x_at | | | | | |
| MPEG1 | 226818_at:918920-HuGene_st:283447- | 4.58E−08 | 1.6464 | 1.86 | 79.5 | 70.8-86.4 |
| | HuGene_st:449729-HuGene_st:301732- | | | | | |
| | HuGene_st:915566-HuGene_st:854136- | | | | | |
| | HuGene_st:226841_at:477225-HuGene_st:1084905- | | | | | |
| | HuGene_st:104883-HuGene_st:342839- | | | | | |
| | HuGene_st:863106-HuGene_st:504453- | | | | | |
| | HuGene_st:44663-HuGene_st:607415- | | | | | |
| | HuGene_st:968224-HuGene_st:990725- | | | | | |
| | HuGene_st:735251-HuGene_st | | | | | |
| IQGAP2 | 185028-HuGene_st:763806-HuGene_st:88647- | 5.06E−08 | 1.6277 | 2.51 | 79.2 | 70.5-86.2 |
| | HuGene_st:93358-HuGene_st:393573- | | | | | |
| | HuGene_st:743251-HuGene_st:1010599- | | | | | |
| | HuGene_st:786301-HuGene_st:1066864- | | | | | |
| | HuGene_st:231977-HuGene_st:370908- | | | | | |
| | HuGene_st:937713-HuGene_st:201743- | | | | | |
| | HuGene_st:243728_at:899180-HuGene_st:981926- | | | | | |
| | HuGene_st:897914-HuGene_st:1095716- | | | | | |
| | HuGene_st:802231-HuGene_st:263364- | | | | | |
| | HuGene_st:986453-HuGene_st:203474_at | | | | | |
| LMOD1 | 611871-HuGene_st:53373-HuGene_st:380885- | 5.55E−08 | 1.5812 | 2.97 | 78.5 | 69.8-85.6 |
| | HuGene_st:958079-HuGene_st:986515- | | | | | |
| | HuGene_st:173411-HuGene_st:616270- | | | | | |
| | HuGene_st:517377-HuGene_st:433277- | | | | | |
| | HuGene_st:287901-HuGene_st:1071756- | | | | | |
| | HuGene_st:180631-HuGene_st:52042- | | | | | |
| | HuGene_st:203766_s_at:732981- | | | | | |
| | HuGene_st:211562_s_at:999216-HuGene_st:993253- | | | | | |
| | HuGene_st:589530-HuGene_st:675886- | | | | | |
| | HuGene_st:945700-HuGene_st | | | | | |
| PGM5 | 810480-HuGene_st:111685-HuGene_st:338926- | 5.87E−08 | 1.6218 | 5.87 | 79.1 | 70.4-86.2 |
| | HuGene_st:81611-HuGene_st:658883- | | | | | |
| | HuGene_st:803066-HuGene_st:208491_s_at:956998- | | | | | |
| | HuGene_st:1095193-HuGene_st:1066912- | | | | | |
| | HuGene_st:1079743-HuGene_st:743259- | | | | | |
| | HuGene_st:429274-HuGene_st:259184- | | | | | |
| | HuGene_st:376761-HuGene_st:249074- | | | | | |
| | HuGene_st:226303_at | | | | | |
| DDR2 | 857174-HuGene_st:297630-HuGene_st:198601- | 5.89E−08 | 1.5612 | 2.61 | 78.2 | 69.5-85.4 |
| | HuGene_st:951531-HuGene_st:341474- | | | | | |
| | HuGene_st:1008685-HuGene_st:1000005- | | | | | |
| | HuGene_st:712988-HuGene_st:263417- | | | | | |
| | HuGene_st:511597-HuGene_st:348176- | | | | | |
| | HuGene_st:928611-HuGene_st:318480- | | | | | |
| | HuGene_st:801516-HuGene_st:849806- | | | | | |
| | HuGene_st:510631-HuGene_st:225442_at:87448- | | | | | |
| | HuGene_st:704299-HuGene_st:227561_at | | | | | |
| PALM2- | 202759_s_at:202760_s_at | 6.28E−08 | 1.613 | 2.17 | 79 | 70.3-86.1 |
| AKAP2 | | | | | | |
| AOC3 | 557248-HuGene_st:209280-HuGene_st:588332- | 6.43E−08 | 1.5565 | 2.92 | 78.2 | 69.3-85.4 |
| | HuGene_st:108003-HuGene_st:960783- | | | | | |
| | HuGene_st:407777-HuGene_st:147438- | | | | | |
| | HuGene_st:548345-HuGene_st:721301- | | | | | |
| | HuGene_st:128882-HuGene_st:340215- | | | | | |
| | HuGene_st:631235-HuGene_st:641187- | | | | | |
| | HuGene_st:117366-HuGene_st:949088- | | | | | |
| | HuGene_st:204894_s_at | | | | | |
| PAPSS2 | 1040734-HuGene_st:893182-HuGene_st:355010- | 6.43E−08 | 1.6163 | 1.97 | 79 | 70.4-86.1 |
| | HuGene_st:808055-HuGene_st:449559- | | | | | |
| | HuGene_st:203059_s_at:710965-HuGene_st:891049- | | | | | |
| | HuGene_st:1050455-HuGene_st:59122- | | | | | |
| | HuGene_st:203058_s_at:203060_s_at:968581- | | | | | |
| | HuGene_st:932781-HuGene_st:291649- | | | | | |
| | HuGene_st:600968-HuGene_st:413041- | | | | | |
| | HuGene_st:247101-HuGene_st:1087256- | | | | | |
| | HuGene_st:237496_at:15324-HuGene_st:675829- | | | | | |
| | HuGene_st:771003-HuGene_st:343109-HuGene_st | | | | | |
| SDPR | 878908-HuGene_st:781527-HuGene_st:331976- | 9.62E−08 | 1.6006 | 2.21 | 78.8 | 70-85.9 |
| | HuGene_st:238150-HuGene_st:306039- | | | | | |
| | HuGene_st:535903-HuGene_st:302361- | | | | | |
| | HuGene_st:1005813-HuGene_st:71118- | | | | | |
| | HuGene_st:992629-HuGene_st:218711_s_at:293110- | | | | | |
| | HuGene_st:779040-HuGene_st:222717_at:970479- | | | | | |
| | HuGene_st:581654-HuGene_st | | | | | |
| DUSP1 | 308761-HuGene_st:904724-HuGene_st:371253- | 1.06E−07 | 1.527 | 2.44 | 77.7 | 68.8-85 |
| | HuGene_st:201041_s_at:918293-HuGene_st:636896- | | | | | |
| | HuGene_st:793780-HuGene_st:714382- | | | | | |
| | HuGene_st:83208-HuGene_st:504846- | | | | | |
| | HuGene_st:1046152-HuGene_st:936429- | | | | | |
| | HuGene_st:226578_s_at:367434-HuGene_st:676144- | | | | | |
| | HuGene_st:57046-HuGene_st:466265- | | | | | |
| | HuGene_st:775780-HuGene_st:747888- | | | | | |
| | HuGene_st:554753-HuGene_st:101136- | | | | | |
| | HuGene_st:829116-HuGene_st:201044_x_at:571046- | | | | | |
| | HuGene_st | | | | | |
| PRKAR2B | 101664-HuGene_st:162600-HuGene_st:426444- | 1.09E−07 | 1.677 | 2.1 | 79.9 | 71.2-86.9 |
| | HuGene_st:988677-HuGene_st:546073- | | | | | |
| | HuGene_st:714756-HuGene_st:683742- | | | | | |
| | HuGene_st:422832-HuGene_st:509601- | | | | | |
| | HuGene_st:898244-HuGene_st:904472- | | | | | |
| | HuGene_st:705909-HuGene_st:348626- | | | | | |
| | HuGene_st:699010-HuGene_st:742973- | | | | | |
| | HuGene_st:283465-HuGene_st:720328- | | | | | |
| | HuGene_st:320328-HuGene_st:210153_s_at:688661- | | | | | |
| | HuGene_st:478012-HuGene_st:373286- | | | | | |
| | HuGene_st:484605-HuGene_st:989810- | | | | | |
| | HuGene_st:280047-HuGene_st:473253- | | | | | |
| | HuGene_st:630331-HuGene_st:1003297- | | | | | |
| | HuGene_st:209397_at:203680_at | | | | | |
| CNTN3 | 267567-HuGene_st:782053-HuGene_st:550157- | 1.20E−07 | 1.6161 | 3.93 | 79 | 70.3-86.1 |
| | HuGene_st:541394-HuGene_st:989173- | | | | | |
| | HuGene_st:15899-HuGene_st:78622- | | | | | |
| | HuGene_st:339738-HuGene_st:585282- | | | | | |
| | HuGene_st:661814-HuGene_st:360715- | | | | | |
| | HuGene_st:695033-HuGene_st:483058- | | | | | |
| | HuGene_st:555394-HuGene_st:315011- | | | | | |
| | HuGene_st:905374-HuGene_st:1067212- | | | | | |
| | HuGene_st:557263-HuGene_st:233502_at:811729- | | | | | |
| | HuGene_st:87414-HuGene_st | | | | | |
| RAB27A | 339168-HuGene_st:340479-HuGene_st:1057756- | 1.22E−07 | 1.5976 | 1.79 | 78.8 | 70-85.9 |
| | HuGene_st:99120-HuGene_st:301308- | | | | | |
| | HuGene_st:309278-HuGene_st:641167- | | | | | |
| | HuGene_st:900707-HuGene_st:961788- | | | | | |
| | HuGene_st:210951_x_at:209515_s_at:924247- | | | | | |
| | HuGene_st:209514_s_at:2432-HuGene_st:405271- | | | | | |
| | HuGene_st:758428-HuGene_st:235766_x_at:391537- | | | | | |
| | HuGene_st:983411-HuGene_st:222294_s_at | | | | | |
| C6orf105 | 669403-HuGene_st:937383-HuGene_st:344202- | 1.38E−07 | 1.6043 | 2.13 | 78.9 | 70.1-85.9 |
| | HuGene_st:229070_at:1088682- | | | | | |
| | HuGene_st:215100_at:287254-HuGene_st:34196- | | | | | |
| | HuGene_st:701140-HuGene_st:461427- | | | | | |
| | HuGene_st:1032543-HuGene_st:237434- | | | | | |
| | HuGene_st:542250-HuGene_st:316541- | | | | | |
| | HuGene_st:179382-HuGene_st:148807- | | | | | |
| | HuGene_st:1029919-HuGene_st:58955- | | | | | |
| | HuGene_st:48477-HuGene_st:869984- | | | | | |
| | HuGene_st:667357-HuGene_st:697774- | | | | | |
| | HuGene_st:164714-HuGene_st:105735-HuGene_st | | | | | |
| MUC12 | 412725-HuGene_st:1083259-HuGene_st:437412- | 1.53E−07 | 1.4566 | 4.89 | 76.7 | 67.7-84.1 |
| | HuGene_st:751451-HuGene_st:498196- | | | | | |
| | HuGene_st:1087628-HuGene_st:214378- | | | | | |
| | HuGene_st:61291-HuGene_st:661525- | | | | | |
| | HuGene_st:678015-HuGene_st:468158- | | | | | |
| | HuGene_st:991377-HuGene_st:280731- | | | | | |
| | HuGene_st:790312-HuGene_st:770658- | | | | | |
| | HuGene_st:56500-HuGene_st:181935- | | | | | |
| | HuGene_st:1013137- | | | | | |
| | HuGene_st:1557906_at:226654_at:1557907_x_at:1537 | | | | | |
| | 51-HuGene_st:765891-HuGene_st:946487- | | | | | |
| | HuGene_st:231814_at:42037-HuGene_st | | | | | |
| AXL | 321003-HuGene_st:546377-HuGene_st:429002- | 1.59E−07 | 1.5776 | 1.71 | 78.5 | 69.7-85.6 |
| | HuGene_st:790260-HuGene_st:796222- | | | | | |
| | HuGene_st:59559-HuGene_st:221741- | | | | | |
| | HuGene_st:669140-HuGene_st:822162- | | | | | |
| | HuGene_st:521036-HuGene_st:202685_s_at:501383- | | | | | |
| | HuGene_st:883192-HuGene_st | | | | | |
| IL1R2 | 377084-HuGene_st:278685-HuGene_st:472031- | 1.73E−07 | 1.5226 | 2.54 | 77.7 | 68.7-84.9 |
| | HuGene_st:792041-HuGene_st:1012886- | | | | | |
| | HuGene_st:568945-HuGene_st:80054- | | | | | |
| | HuGene_st:866918-HuGene_st:866883- | | | | | |
| | HuGene_st:17623-HuGene_st:314768- | | | | | |
| | HuGene_st:326744-HuGene_st:211372_s_at:888405- | | | | | |
| | HuGene_st:205403_at:834475-HuGene_st:386354- | | | | | |
| | HuGene_st:411355-HuGene_st:862981- | | | | | |
| | HuGene_st:70227-HuGene_st | | | | | |
| TSC22D3 | 235364_at:1044209-HuGene_st:519020- | 1.85E−07 | 1.6337 | 3.1 | 79.3 | 70.6-86.3 |
| | HuGene_st:427467-HuGene_st:669553- | | | | | |
| | HuGene_st:722843-HuGene_st:208763_s_at:126162- | | | | | |
| | HuGene_st:207001_x_at:654578-HuGene_st:146515- | | | | | |
| | HuGene_st:1100721-HuGene_st:823307- | | | | | |
| | HuGene_st:899494-HuGene_st:952685- | | | | | |
| | HuGene_st:235399-HuGene_st:802521- | | | | | |
| | HuGene_st:126175-HuGene_st:722700-HuGene_st | | | | | |
| GPA33 | 169349-HuGene_st:772424-HuGene_st:482659- | 1.86E−07 | 1.7041 | 1.61 | 80.3 | 71.7-87.2 |
| | HuGene_st:1053088-HuGene_st:221229- | | | | | |
| | HuGene_st:205929_at:21623-HuGene_st:352510- | | | | | |
| | HuGene_st:431411-HuGene_st:507225- | | | | | |
| | HuGene_st:362352-HuGene_st:492326- | | | | | |
| | HuGene_st:555302-HuGene_st:812966- | | | | | |
| | HuGene_st:207883-HuGene_st:401745- | | | | | |
| | HuGene_st:630242-HuGene_st:212295- | | | | | |
| | HuGene_st:1097025-HuGene_st:157847-HuGene_st | | | | | |
| IDH3A | 202069_s_at:300578-HuGene_st:202070_s_at:342767- | 2.08E−07 | 1.567 | 1.75 | 78.3 | 69.6-85.5 |
| | HuGene_st:8965-HuGene_st:1079553- | | | | | |
| | HuGene_st:221816-HuGene_st:1042110- | | | | | |
| | HuGene_st:647088-HuGene_st:670662- | | | | | |
| | HuGene_st:53656-HuGene_st:572196- | | | | | |
| | HuGene_st:607148-HuGene_st:463534- | | | | | |
| | HuGene_st:784198-HuGene_st:859733- | | | | | |
| | HuGene_st:221939-HuGene_st:606301- | | | | | |
| | HuGene_st:546210-HuGene_st:475783- | | | | | |
| | HuGene_st:1084692-HuGene_st:538386-HuGene_st | | | | | |
| MATN2 | 902099-HuGene_st:1065921-HuGene_st:452682- | 2.11E−07 | 1.5084 | 2.23 | 77.5 | 68.6-84.8 |
| | HuGene_st:1012649-HuGene_st:1019476- | | | | | |
| | HuGene_st:88797-HuGene_st:986521- | | | | | |
| | HuGene_st:181168-HuGene_st:102298- | | | | | |
| | HuGene_st:757359-HuGene_st:859886- | | | | | |
| | HuGene_st:540941-HuGene_st:202350_s_at:326901- | | | | | |
| | HuGene_st:781583-HuGene_st:498255- | | | | | |
| | HuGene_st:212041-HuGene_st | | | | | |
| MT2A | 723245-HuGene_st:710161-HuGene_st:133506- | 2.49E−07 | 1.6459 | 2.41 | 79.5 | 70.8-86.5 |
| | HuGene_st:90770-HuGene_st:309654- | | | | | |
| | HuGene_st:194391-HuGene_st:692585- | | | | | |
| | HuGene_st:117983-HuGene_st:858756- | | | | | |
| | HuGene_st:123483-HuGene_st:296152- | | | | | |
| | HuGene_st:217546_at:239766-HuGene_st:46711- | | | | | |
| | HuGene_st:1057926-HuGene_st:611729- | | | | | |
| | HuGene_st:1048755-HuGene_st:519540- | | | | | |
| | HuGene_st:284543-HuGene_st:847898- | | | | | |
| | HuGene_st:648660-HuGene_st:977125- | | | | | |
| | HuGene_st:743580-HuGene_st:327541- | | | | | |
| | HuGene_st:948505-HuGene_st:6546- | | | | | |
| | HuGene_st:84521-HuGene_st:907619- | | | | | |
| | HuGene_st:939779-HuGene_st:561550- | | | | | |
| | HuGene_st:76641-HuGene_st:1000258- | | | | | |
| | HuGene_st:447330-HuGene_st:983649- | | | | | |
| | HuGene_st:613324-HuGene_st:846448- | | | | | |
| | HuGene_st:304444-HuGene_st:212859_x_at:40069- | | | | | |
| | HuGene_st:212884-HuGene_st:575774- | | | | | |
| | HuGene_st:427914-HuGene_st:56303- | | | | | |
| | HuGene_st:213349-HuGene_st:721776- | | | | | |
| | HuGene_st:212185_x_at:739002-HuGene_st:902404- | | | | | |
| | HuGene_st:483985:HuGene_st:91794- | | | | | |
| | HuGene_st:137880-HuGene_st:317864- | | | | | |
| | HuGene_st:203291-HuGene_st:66799- | | | | | |
| | HuGene_st:160991-HuGene_st:216336_x_at:310066- | | | | | |
| | HuGene_st:1079767-HuGene_st:781831- | | | | | |
| | HuGene_st:1095705-HuGene_st:534398- | | | | | |
| | HuGene_st:467500-HuGene_st:491335- | | | | | |
| | HuGene_st:66800-HuGene_st:485318- | | | | | |
| | HuGene_st:90966-HuGene_st:365104- | | | | | |
| | HuGene_st:79259-HuGene_st:495714- | | | | | |
| | HuGene_st:1095744-HuGene_st:102946- | | | | | |
| | HuGene_st:477858-HuGene_st:771030- | | | | | |
| | HuGene_st:675707-HuGene_st:854293- | | | | | |
| | HuGene_st:160013-HuGene_st:320514- | | | | | |
| | HuGene_st:1047433-HuGene_st:890901- | | | | | |
| | HuGene_st:230953-HuGene_st:749925- | | | | | |
| | HuGene_st:19613-HuGene_st:166551- | | | | | |
| | HuGene_st:574267-HuGene_st:623210- | | | | | |
| | HuGene_st:1006466-HuGene_st:900368- | | | | | |
| | HuGene_st:760559-HuGene_st:446223- | | | | | |
| | HuGene_st:205427-HuGene_st:983813- | | | | | |
| | HuGene_st:435034-HuGene_st:250747-HuGene_st | | | | | |
| MT1M | 723245-HuGene_st:710161-HuGene_st:133506- | 2.49E−07 | 1.6419 | 2.41 | 79.4 | 70.7-86.4 |
| | HuGene_st:90770-HuGene_st:309654- | | | | | |
| | HuGene_st:194391-HuGene_st:692585- | | | | | |
| | HuGene_st:117983-HuGene_st:858756- | | | | | |
| | HuGene_st:123483-HuGene_st:296152- | | | | | |
| | HuGene_st:217546_at:239766-HuGene_st:46711- | | | | | |
| | HuGene_st:1057926-HuGene_st:611729- | | | | | |
| | HuGene_st:1048755-HuGene_st:519540- | | | | | |
| | HuGene_st:284543-HuGene_st:847898- | | | | | |
| | HuGene_st:648660-HuGene_st:977125- | | | | | |
| | HuGene_st:743580-HuGene_st:327541- | | | | | |
| | HuGene_st:948505-HuGene_st:6546- | | | | | |
| | HuGene_st:84521-HuGene_st:907619- | | | | | |
| | HuGene_st:939779-HuGene_st:561550- | | | | | |
| | HuGene_st:76641-HuGene_st:1000258- | | | | | |
| | HuGene_st:447330-HuGene_st:983649- | | | | | |
| | HuGene_st:613324-HuGene_st:846448- | | | | | |
| | HuGene_st:304444-HuGene_st:212859_x_at:40069- | | | | | |
| | HuGene_st:212884-HuGene_st:575774- | | | | | |
| | HuGene_st:427914-HuGene_st:56303- | | | | | |
| | HuGene_st:213349-HuGene_st:721776- | | | | | |
| | HuGene_st:212185_x_at:739002-HuGene_st:902404- | | | | | |
| | HuGene_st:483985-HuGene_st:91794- | | | | | |
| | HuGene_st:137880-HuGene_st:317864- | | | | | |
| | HuGene_st:203291-HuGene_st:66799- | | | | | |
| | HuGene_st:160991-HuGene_st:216336_x_at:310066- | | | | | |
| | HuGene_st:1079767-HuGene_st:781831- | | | | | |
| | HuGene_st:1095705-HuGene_st:534398- | | | | | |
| | HuGene_st:467500-HuGene_st:491335- | | | | | |
| | HuGene_st:66800-HuGene_st:485318- | | | | | |
| | HuGene_st:90966-HuGene_st:365104- | | | | | |
| | HuGene_st:79259-HuGene_st:495714- | | | | | |
| | HuGene_st:1095744-HuGene_st:102946- | | | | | |
| | HuGene_st:477858-HuGene_st:771030- | | | | | |
| | HuGene_st:675707-HuGene_st:854293- | | | | | |
| | HuGene_st:160013-HuGene_st:320514- | | | | | |
| | HuGene_st:1047433-HuGene_st:890901- | | | | | |
| | HuGene_st:230953-HuGene_st:749925- | | | | | |
| | HuGene_st:19613-HuGene_st:166551- | | | | | |
| | HuGene_st:574267-HuGene_st:623210- | | | | | |
| | HuGene_st:1006466-HuGene_st:900368- | | | | | |
| | HuGene_st:760559-HuGene_st:446223- | | | | | |
| | HuGene_st:205427-HuGene_st:983813- | | | | | |
| | HuGene_st:435034-HuGene_st:250747-HuGene_st | | | | | |
| IGL@ | 173623-HuGene_st:408026-HuGene_st:38807- | 2.50E−07 | 1.6337 | 3.1 | 79.3 | 70.7-86.3 |
| | HuGene_st | | | | | |
| HSPB8 | 688528-HuGene_st:311091-HuGene_st:415722- | 2.61E−07 | 1.5121 | 3.85 | 77.5 | 68.7-84.8 |
| | HuGene_st:56376-HuGene_st:79205- | | | | | |
| | HuGene_st:499751-HuGene_st:214231- | | | | | |
| | HuGene_st:225808-HuGene_st:174359- | | | | | |
| | HuGene_st:1083550-HuGene_st:221667_s_at:662744- | | | | | |
| | HuGene_st:346589-HuGene_st | | | | | |
| A2M | 897456-HuGene_st:217757_at:164005- | 2.72E−07 | 1.5968 | 1.64 | 78.8 | 69.9-85.9 |
| | HuGene_st:848096-HuGene_st:244657- | | | | | |
| | HuGene_st:317437-HuGene_st:330214- | | | | | |
| | HuGene_st:814658-HuGene_st:1100520- | | | | | |
| | HuGene_st:451567-HuGene_st:559737- | | | | | |
| | HuGene_st:600508-HuGene_st:730036- | | | | | |
| | HuGene_st:385395-HuGene_st:833129- | | | | | |
| | HuGene_st:101038-HuGene_st:195639- | | | | | |
| | HuGene_st:371583-HuGene_st:808161- | | | | | |
| | HuGene_st:893977-HuGene_st:1558450_at:167145- | | | | | |
| | HuGene_st:660806-HuGene_st | | | | | |
| MS4A7 | 803709-HuGene_st:372940-HuGene_st:558657- | 2.92E−07 | 1.5398 | 2.27 | 77.9 | 69.1-85.2 |
| | HuGene_st:758990-HuGene_st:279234- | | | | | |
| | HuGene_st:716878-HuGene_st:228770- | | | | | |
| | HuGene_st:77553-HuGene_st:212226- | | | | | |
| | HuGene_st:148024-HuGene_st:727350- | | | | | |
| | HuGene_st:223344_s_at:223343_at:224358_s_at:932003- | | | | | |
| | HuGene_st:584291-HuGene_st:16857- | | | | | |
| | HuGene_st:751363-HuGene_st:120594- | | | | | |
| | HuGene_st:66810-HuGene_st:473741-HuGene_st | | | | | |
| PLOD2 | 366970-HuGene_st:202619_s_at:557917- | 3.06E−07 | 1.5569 | 2.33 | 78.2 | 69.3-85.4 |
| | HuGene_st:371377-HuGene_st:606385- | | | | | |
| | HuGene_st:54079-HuGene_st:976248- | | | | | |
| | HuGene_st:572307-HuGene_st:716158- | | | | | |
| | HuGene_st:754684-HuGene_st:255595- | | | | | |
| | HuGene_st:17343-HuGene_st:124945- | | | | | |
| | HuGene_st:770320-HuGene_st:704827- | | | | | |
| | HuGene_st:202620_s_at:309852-HuGene_st:509273- | | | | | |
| | HuGene_st:703772-HuGene_st:1082989- | | | | | |
| | HuGene_st:550147-HuGene_st:740582- | | | | | |
| | HuGene_st:6711-HuGene_st | | | | | |
| UGT2B17 | 357315-HuGene_st:493733-HuGene_st:353886- | 3.75E−07 | 1.4818 | 4.88 | 77.1 | 68.2-84.5 |
| | HuGene_st:207245_at:59042-HuGene_st:1025125- | | | | | |
| | HuGene_st | | | | | |
| PMP22 | 1086412-HuGene_st:139615-HuGene_st:864327- | 3.97E−07 | 1.5173 | 2.23 | 77.6 | 68.8-84.9 |
| | HuGene_st:1565637_at:493430-HuGene_st:640953- | | | | | |
| | HuGene_st:309874-HuGene_st:1072798- | | | | | |
| | HuGene_st:761816-HuGene_st:136620- | | | | | |
| | HuGene_st:441104-HuGene_st:97712- | | | | | |
| | HuGene_st:800086-HuGene_st:352501- | | | | | |
| | HuGene_st:363233-HuGene_st:210139_s_at:48107- | | | | | |
| | HuGene_st:338800-HuGene_st:532682- | | | | | |
| | HuGene_st:988715-HuGene_st:853970-HuGene_st | | | | | |
| PLN | 204938_s_at:418214-HuGene_st:769837- | 4.14E−07 | 1.5464 | 2.01 | 78 | 69.2-85.2 |
| | HuGene_st:603329-HuGene_st:1048096- | | | | | |
| | HuGene_st:944510-HuGene_st:430402- | | | | | |
| | HuGene_st:86633-HuGene_st:795093- | | | | | |
| | HuGene_st:377757-HuGene_st:770145- | | | | | |
| | HuGene_st:920628-HuGene_st:594048- | | | | | |
| | HuGene_st:800583-HuGene_st:204939_s_at:556723- | | | | | |
| | HuGene_st:818339-HuGene_st:175524- | | | | | |
| | HuGene_st:498924-HuGene_st:204940_at | | | | | |
| CNN1 | 337187-HuGene_st:787686-HuGene_st:532503- | 4.77E−07 | 1.4318 | 4.56 | 76.3 | 67.2-83.8 |
| | HuGene_st:41547-HuGene_st:236587- | | | | | |
| | HuGene_st:809046-HuGene_st:674638- | | | | | |
| | HuGene_st:1055006-HuGene_st:617683- | | | | | |
| | HuGene_st:769218-HuGene_st:373491- | | | | | |
| | HuGene_st:609613-HuGene_st:402708- | | | | | |
| | HuGene_st:871546-HuGene_st:203951_at:880804- | | | | | |
| | HuGene_st:1091224-HuGene_st:911860- | | | | | |
| | HuGene_st:1090431-HuGene_st | | | | | |
| ITM2A | 391687-HuGene_st:1059695-HuGene_st:12009- | 5.27E−07 | 1.4958 | 2.77 | 77.3 | 68.3-84.6 |
| | HuGene_st:677746-HuGene_st:258271- | | | | | |
| | HuGene_st:676659-HuGene_st:496836- | | | | | |
| | HuGene_st:452608-HuGene_st:1059257- | | | | | |
| | HuGene_st:47720-HuGene_st:105265- | | | | | |
| | HuGene_st:657686-HuGene_st:68702- | | | | | |
| | HuGene_st:115530- | | | | | |
| | HuGene_st:202747_s_at:202746_at | | | | | |
| KCNMA1 | 554163-HuGene_st:716361-HuGene_st:403072- | 6.86E−07 | 1.497 | 2.37 | 77.3 | 68.4-84.6 |
| | HuGene_st:63347-HuGene_st:587079- | | | | | |
| | HuGene_st:256038-HuGene_st:172619- | | | | | |
| | HuGene_st:918468-HuGene_st:94420- | | | | | |
| | HuGene_st:399013-HuGene_st:414900- | | | | | |
| | HuGene_st:307320-HuGene_st:906761- | | | | | |
| | HuGene_st:115093-HuGene_st:221583_s_at:745071- | | | | | |
| | HuGene_st:915037-HuGene_st:343951- | | | | | |
| | HuGene_st:1569763_at:446685-HuGene_st:652192- | | | | | |
| | HuGene_st | | | | | |
| XDH | 919455-HuGene_st:105269-HuGene_st:106986- | 8.13E−07 | 1.4727 | 2.03 | 76.9 | 67.9-84.3 |
| | HuGene_st:972900-HuGene_st:210301_at:716491- | | | | | |
| | HuGene_st:667307-HuGene_st:790507- | | | | | |
| | HuGene_st:800140-HuGene_st:328962- | | | | | |
| | HuGene_st:355792-HuGene_st:707858- | | | | | |
| | HuGene_st:731429-HuGene_st:403069- | | | | | |
| | HuGene_st:215338-HuGene_st:101916- | | | | | |
| | HuGene_st:48855-HuGene_st:895641- | | | | | |
| | HuGene_st:68212-HuGene_st:287011- | | | | | |
| | HuGene_st:1082168-HuGene_st:241994_at:709200- | | | | | |
| | HuGene_st | | | | | |
| PDLIM3 | 659306-HuGene_st:9491-HuGene_st:787864- | 8.20E−07 | 1.4799 | 2.38 | 77 | 68.2-84.4 |
| | HuGene_st:482305-HuGene_st:810161- | | | | | |
| | HuGene_st:1037974-HuGene_st:353413- | | | | | |
| | HuGene_st:731754-HuGene_st:733690- | | | | | |
| | HuGene_st:209621_s_at:394037-HuGene_st:957822- | | | | | |
| | HuGene_st:67576-HuGene_st:369461- | | | | | |
| | HuGene_st:768470-HuGene_st:489488- | | | | | |
| | HuGene_st:711969-HuGene_st:1569564_at:1077054- | | | | | |
| | HuGene_st:210170_at:238592_at | | | | | |
| MT1X | 788358-HuGene_st:20578-HuGene_st:92311- | 9.84E−07 | 1.4411 | 4.35 | 76.4 | 67.5-83.9 |
| | HuGene_st:501820-HuGene_st:208581_x_at:73687- | | | | | |
| | HuGene_st:905260-HuGene_st:1087816- | | | | | |
| | HuGene_st:204326_x_at:96216-HuGene_st:764373- | | | | | |
| | HuGene_st:103649-HuGene_st:40373- | | | | | |
| | HuGene_st:198342-HuGene_st | | | | | |
| CALD1 | 796040-HuGene_st:839187-HuGene_st:412659- | 1.02E−06 | 1.4584 | 3.29 | 76.7 | 67.7-84.1 |
| | HuGene_st:1023569-HuGene_st:450656- | | | | | |
| | HuGene_st:212077_at:201616_s_at:816439- | | | | | |
| | HuGene_st:165931-HuGene_st:201617_x_at:576686- | | | | | |
| | HuGene_st:1094139-HuGene_st:201615_x_at:519079- | | | | | |
| | HuGene_st:558226-HuGene_st:755661- | | | | | |
| | HuGene_st:243084_at:318906-HuGene_st:688034- | | | | | |
| | HuGene_st:215199_at | | | | | |
| MSRB3 | 483115-HuGene_st:439736-HuGene_st:1096860- | 1.20E−06 | 1.4754 | 2.24 | 77 | 68-84.4 |
| | HuGene_st:644381-HuGene_st:225790_at:306630- | | | | | |
| | HuGene_st:1073255-HuGene_st:121556- | | | | | |
| | HuGene_st:126154-HuGene_st:72517- | | | | | |
| | HuGene_st:480208-HuGene_st:331986- | | | | | |
| | HuGene_st:294511-HuGene_st:204701- | | | | | |
| | HuGene_st:1554127_s_at:42731-HuGene_st:675396- | | | | | |
| | HuGene_st:174560-HuGene_st:1566481_at | | | | | |
| SPON1 | 209437_s_at:209436_at:892181-HuGene_st:960315- | 1.46E−06 | 1.4862 | 1.88 | 77.1 | 68.2-84.5 |
| | HuGene_st:377887-HuGene_st:253667- | | | | | |
| | HuGene_st:48054-HuGene_st:1008441- | | | | | |
| | HuGene_st:979120-HuGene_st:213994_s_at:811254- | | | | | |
| | HuGene_st:928583-HuGene_st:405680- | | | | | |
| | HuGene_st:963535-HuGene_st:836638- | | | | | |
| | HuGene_st:1033364-HuGene_st:965345- | | | | | |
| | HuGene_st:936102-HuGene_st:555640- | | | | | |
| | HuGene_st:22053-HuGene_st:234367- | | | | | |
| | HuGene_st:224488_s_at:327210-HuGene_st:481375- | | | | | |
| | HuGene_st:213993_at:1013182-HuGene_st | | | | | |
| C2orf40 | 1011921-HuGene_st:304065-HuGene_st:441303- | 1.69E−06 | 1.4159 | 2.97 | 76.1 | 67-83.6 |
| | HuGene_st:1001854-HuGene_st:999754- | | | | | |
| | HuGene_st:893886-HuGene_st:951843- | | | | | |
| | HuGene_st:375926-HuGene_st | | | | | |
| SORBS1 | 1041567-HuGene_st:83451-HuGene_st:29118- | 2.18E−06 | 1.4269 | 2.54 | 76.2 | 67.2-83.7 |
| | HuGene_st:222513_s_at:617821-HuGene_st:56685- | | | | | |
| | HuGene_st:280855-HuGene_st:67922- | | | | | |
| | HuGene_st:1028489-HuGene_st:611267- | | | | | |
| | HuGene_st:931477-HuGene_st:66752- | | | | | |
| | HuGene_st:618734-HuGene_st:624287- | | | | | |
| | HuGene_st:211819_s_at:581936- | | | | | |
| | HuGene_st:218087_s_at:1039879-HuGene_st:45815- | | | | | |
| | HuGene_st:186556-HuGene_st:628496- | | | | | |
| | HuGene_st:242736_at | | | | | |
| C4orf34 | 576095-HuGene_st:1061109-HuGene_st:698934- | 2.55E−06 | 1.3851 | 1.95 | 75.6 | 66.5-83.1 |
| | HuGene_st:634224-HuGene_st:854845- | | | | | |
| | HuGene_st:18233-HuGene_st:275684- | | | | | |
| | HuGene_st:784949-HuGene_st:346086- | | | | | |
| | HuGene_st:91468-HuGene_st:936558- | | | | | |
| | HuGene_st:163564-HuGene_st:614699- | | | | | |
| | HuGene_st:428090-HuGene_st:340671- | | | | | |
| | HuGene_st:90795-HuGene_st:823553- | | | | | |
| | HuGene_st:431110-HuGene_st:719502- | | | | | |
| | HuGene_st:850208-HuGene_st:224990_at:542653- | | | | | |
| | HuGene_st | | | | | |
| PDGFRA | 782752-HuGene_st:789846-HuGene_st:116091- | 2.94E−06 | 1.4819 | 1.37 | 77.1 | 68.1-84.4 |
| | HuGene_st:860506-HuGene_st:537532- | | | | | |
| | HuGene_st:112478-HuGene_st:97681- | | | | | |
| | HuGene_st:514113-HuGene_st:561375- | | | | | |
| | HuGene_st:115277-HuGene_st:240764- | | | | | |
| | HuGene_st:578952-HuGene_st:723105- | | | | | |
| | HuGene_st:230638-HuGene_st:544163- | | | | | |
| | HuGene_st:50957-HuGene_st:468599- | | | | | |
| | HuGene_st:328136-HuGene_st:346391- | | | | | |
| | HuGene_st:203131_at:853019-HuGene_st | | | | | |
| FGFR2 | 476373-HuGene_st:92078-HuGene_st:969020- | 3.55E−06 | 1.527 | 1.31 | 77.7 | 68.9-85 |
| | HuGene_st:1022426-HuGene_st:203639_s_at:24514- | | | | | |
| | HuGene_st:614945-HuGene_st:211398_at:1097953- | | | | | |
| | HuGene_st:117962-HuGene_st:777874- | | | | | |
| | HuGene_st:829764-HuGene_st:868500- | | | | | |
| | HuGene_st:211401_s_at:403546-HuGene_st:850416- | | | | | |
| | HuGene_st:208234_x_at:537762-HuGene_st:132866- | | | | | |
| | HuGene_st:426700-HuGene_st:208228_s_at:831045- | | | | | |
| | HuGene_st:203638_s_at:240913_at:230842_at | | | | | |
| PPP1R12B | 23543-HuGene_st:654289-HuGene_st:947526- | 4.26E−06 | 1.3592 | 2.21 | 75.2 | 66.1-82.8 |
| | HuGene_st:714709-HuGene_st:544138- | | | | | |
| | HuGene_st:1557553_at:227411- | | | | | |
| | HuGene_st:201958_s_at:1559911_at:74838- | | | | | |
| | HuGene_st:201957_at:233700_at:67323- | | | | | |
| | HuGene_st:767707-HuGene_st:224270_at:50561- | | | | | |
| | HuGene_st:233359-HuGene_st:565179- | | | | | |
| | HuGene_st:317428-HuGene_st:442074- | | | | | |
| | HuGene_st:591561-HuGene_st | | | | | |
| FAM129A | 148319-HuGene_st:19042-HuGene_st:1075897- | 4.59E−06 | 1.4144 | 1.8 | 76 | 67-83.5 |
| | HuGene_st:49110-HuGene_st:245230- | | | | | |
| | HuGene_st:154650-HuGene_st:203917- | | | | | |
| | HuGene_st:503184-HuGene_st:373453- | | | | | |
| | HuGene_st:432126-HuGene_st:786874- | | | | | |
| | HuGene_st:648379-HuGene_st:1033222- | | | | | |
| | HuGene_st:217966_s_at:574775- | | | | | |
| | HuGene_st:217967_s_at:156986-HuGene_st:9160- | | | | | |
| | HuGene_st:212626-HuGene_st:935377- | | | | | |
| | HuGene_st:921695-HuGene_st:1053262-HuGene_st | | | | | |
| POSTN | 210809_s_at:297712- | 4.94E−06 | 1.3944 | 2.25 | 75.7 | 66.7-83.3 |
| | HuGene_st:1555778_a_at:608435-HuGene_st:743877- | | | | | |
| | HuGene_st:753556-HuGene_st:388659- | | | | | |
| | HuGene_st:954838-HuGene_st:724846- | | | | | |
| | HuGene_st:588010-HuGene_st:713459- | | | | | |
| | HuGene_st:713707-HuGene_st:106590- | | | | | |
| | HuGene_st:538299-HuGene_st:649547- | | | | | |
| | HuGene_st:13993-HuGene_st:417058- | | | | | |
| | HuGene_st:636479-HuGene_st:228481_at:874100- | | | | | |
| | HuGene_st:776445-HuGene_st:779754-HuGene_st | | | | | |
| ATP8B1 | 1088077-HuGene_st:699379-HuGene_st:81949- | 5.73E−06 | 1.3113 | 1.67 | 74.4 | 65.3-82.1 |
| | HuGene_st:120481-HuGene_st:1006809- | | | | | |
| | HuGene_st:960479-HuGene_st:1033226- | | | | | |
| | HuGene_st:854521-HuGene_st:258436- | | | | | |
| | HuGene_st:224444-HuGene_st:958640- | | | | | |
| | HuGene_st:670830-HuGene_st:734950- | | | | | |
| | HuGene_st:61644-HuGene_st:354636- | | | | | |
| | HuGene_st:308159-HuGene_st:645299- | | | | | |
| | HuGene_st:306422-HuGene_st:223980- | | | | | |
| | HuGene_st:772656-HuGene_st:226302_at:745109- | | | | | |
| | HuGene_st:1011843-HuGene_st | | | | | |
| CCL28 | 224240_s_at:155321-HuGene_st:780816- | 6.01E−06 | 1.494 | 1.53 | 77.2 | 68.4-84.6 |
| | HuGene_st:224027_at:356805-HuGene_st:120679- | | | | | |
| | HuGene_st:525328-HuGene_st:623557- | | | | | |
| | HuGene_st:238750_at:519318-HuGene_st:956859- | | | | | |
| | HuGene_st:251110-HuGene_st:331562- | | | | | |
| | HuGene_st:224868-HuGene_st:28476- | | | | | |
| | HuGene_st:153501-HuGene_st:242217- | | | | | |
| | HuGene_st:821675-HuGene_st:451844- | | | | | |
| | HuGene_st:513548-HuGene_st:773827- | | | | | |
| | HuGene_st:398738-HuGene_st | | | | | |
| NEXN | 184663-HuGene_st:720053-HuGene_st:539444- | 6.83E−06 | 1.363 | 2.23 | 75.2 | 66.1-82.9 |
| | HuGene_st:311368-HuGene_st:260384- | | | | | |
| | HuGene_st:56754-HuGene_st:1040436- | | | | | |
| | HuGene_st:1064626-HuGene_st:292849- | | | | | |
| | HuGene_st:453309-HuGene_st:804402- | | | | | |
| | HuGene_st:500301-HuGene_st:248505- | | | | | |
| | HuGene_st:402104-HuGene_st:1552309_a_at:941387- | | | | | |
| | HuGene_st:709246-HuGene_st:226103_at | | | | | |
| CTGF | NA | 6.96E−06 | 1.4786 | 1.3 | 77 | 68.1-84.4 |
| MFSD4 | 15050-HuGene_st:785959-HuGene_st:884375- | 7.19E−06 | 1.3233 | 2.12 | 74.6 | 65.5-82.3 |
| | HuGene_st:75016-HuGene_st:235772- | | | | | |
| | HuGene_st:185211-HuGene_st:539224- | | | | | |
| | HuGene_st:871027-HuGene_st:855473- | | | | | |
| | HuGene_st:728207-HuGene_st:242782- | | | | | |
| | HuGene_st:711626-HuGene_st:484086- | | | | | |
| | HuGene_st:242372_s_at:238862_at:229254_at:656926- | | | | | |
| | HuGene_st:767273-HuGene_st:184295- | | | | | |
| | HuGene_st:697889-HuGene_st:1566569_at:708165- | | | | | |
| | HuGene_st | | | | | |
| ASPN | 787638-HuGene_st:567513-HuGene_st:499513- | 7.45E−06 | 1.3261 | 2.74 | 74.6 | 65.5-82.3 |
| | HuGene_st:546047-HuGene_st:1055545- | | | | | |
| | HuGene_st:720939-HuGene_st:835521- | | | | | |
| | HuGene_st:640448-HuGene_st:673620- | | | | | |
| | HuGene_st:821057-HuGene_st:248224- | | | | | |
| | HuGene_st:447994-HuGene_st:407869- | | | | | |
| | HuGene_st:510872-HuGene_st:766560-HuGene_st | | | | | |
| DUSP5 | 25091-HuGene_st:352326-HuGene_st:119170- | 8.60E−06 | 1.334 | 1.91 | 74.8 | 65.6-82.4 |
| | HuGene_st:292093-HuGene_st:209457_at:899442- | | | | | |
| | HuGene_st:171695-HuGene_st:331265- | | | | | |
| | HuGene_st:991652-HuGene_st:242476- | | | | | |
| | HuGene_st:430239-HuGene_st:1070934- | | | | | |
| | HuGene_st:555959-HuGene_st | | | | | |
| CRISPLD2 | 314440-HuGene_st:395147-HuGene_st:429916- | 9.24E−06 | 1.3669 | 1.64 | 75.3 | 66.2-82.9 |
| | HuGene_st:763823-HuGene_st:729858- | | | | | |
| | HuGene_st:1555809_at:909486-HuGene_st:239091- | | | | | |
| | HuGene_st:418565-HuGene_st:949339- | | | | | |
| | HuGene_st:658945-HuGene_st:276173- | | | | | |
| | HuGene_st:96079-HuGene_st | | | | | |
| FOXF2 | 589126-HuGene_st:856761-HuGene_st:145881- | 9.50E−06 | 1.3047 | 2.08 | 74.3 | 65.2-82.1 |
| | HuGene_st:915587-HuGene_st:1088669- | | | | | |
| | HuGene_st:81821-HuGene_st:206377_at:575658- | | | | | |
| | HuGene_st:494266-HuGene_st:813114- | | | | | |
| | HuGene_st:613721-HuGene_st | | | | | |
| UGT2B15 | 500573-HuGene_st:352298-HuGene_st:770027- | 9.73E−06 | 1.3469 | 5.72 | 75 | 65.9-82.6 |
| | HuGene_st:207392_x_at:31342-HuGene_st:829850- | | | | | |
| | HuGene_st:217175_at:57112-HuGene_st:1062564- | | | | | |
| | HuGene_st:951114-HuGene_st:844819- | | | | | |
| | HuGene_st:841767-HuGene_st:57590- | | | | | |
| | HuGene_st:504268-HuGene_st:27324- | | | | | |
| | HuGene_st:340207-HuGene_st:102754- | | | | | |
| | HuGene_st:1055412-HuGene_st | | | | | |
| ACTG2 | 667818-HuGene_st:500390-HuGene_st:979342- | 1.07E−05 | 1.3992 | 1.82 | 75.8 | 66.8-83.4 |
| | HuGene_st:560814-HuGene_st:801310- | | | | | |
| | HuGene_st:552015-HuGene_st:972136- | | | | | |
| | HuGene_st:617230-HuGene_st:409424- | | | | | |
| | HuGene_st:15155-HuGene_st:416391- | | | | | |
| | HuGene_st:800450-HuGene_st:902303- | | | | | |
| | HuGene_st:647647-HuGene_st:123624- | | | | | |
| | HuGene_st:202274_at:586898-HuGene_st:431150- | | | | | |
| | HuGene_st:247321-HuGene_st:735136- | | | | | |
| | HuGene_st:986398-HuGene_st:261974- | | | | | |
| | HuGene_st:241148_at:575416-HuGene_st | | | | | |
| CMBL | 157156-HuGene_st:305010- | 1.09E−05 | 1.3583 | 1.58 | 75.1 | 66.1-82.8 |
| | HuGene_st:227522_at:553921-HuGene_st:529224- | | | | | |
| | HuGene_st:1032780-HuGene_st:204886- | | | | | |
| | HuGene_st:623086-HuGene_st:451862- | | | | | |
| | HuGene_st:537678-HuGene_st:918595- | | | | | |
| | HuGene_st:178105-HuGene_st:417861- | | | | | |
| | HuGene_st:230995_at:986212-HuGene_st:405624- | | | | | |
| | HuGene_st:424469-HuGene_st:836099- | | | | | |
| | HuGene_st:665079-HuGene_st:234981_x_at | | | | | |
| KCTD12 | 509699-HuGene_st:77199- | 1.44E−05 | 1.3636 | 1.36 | 75.2 | 66.2-82.8 |
| | HuGene_st:212188_at:229632-HuGene_st:502611- | | | | | |
| | HuGene_st:931430-HuGene_st:908582- | | | | | |
| | HuGene_st:769157-HuGene_st:312044- | | | | | |
| | HuGene_st:212192_at:43415-HuGene_st:412026- | | | | | |
| | HuGene_st:247270-HuGene_st:770750- | | | | | |
| | HuGene_st:827060-HuGene_st:714432-HuGene_st | | | | | |
| MRGPRF | 717973-HuGene_st:226058-HuGene_st:541082- | 1.52E−05 | 1.2986 | 2.99 | 74.2 | 65.1-82 |
| | HuGene_st:60861-HuGene_st:1024643- | | | | | |
| | HuGene_st:354954-HuGene_st:1099895- | | | | | |
| | HuGene_st:672489-HuGene_st:489062- | | | | | |
| | HuGene_st:435421-HuGene_st:227727_at:99658- | | | | | |
| | HuGene_st | | | | | |
| C20orf118 | 235964_x_at:234987_at:235529_x_at:174500- | 1.75E−05 | 1.4284 | 1.3 | 76.2 | 67.3-83.8 |
| | HuGene_st:168513-HuGene_st:312879- | | | | | |
| | HuGene_st:57294-HuGene_st:748530- | | | | | |
| | HuGene_st:24012-HuGene_st:219008- | | | | | |
| | HuGene_st:471274-HuGene_st:1086281- | | | | | |
| | HuGene_st:740018-HuGene_st:233829_at | | | | | |
| TMEM47 | 20487-HuGene_st:587019-HuGene_st:458329- | 1.86E−05 | 1.301 | 2.02 | 74.2 | 65.1-82 |
| | HuGene_st:944333-HuGene_st:292459- | | | | | |
| | HuGene_st:436914-HuGene_st:632184- | | | | | |
| | HuGene_st:209655_s_at:885991-HuGene_st:33524- | | | | | |
| | HuGene_st:528167-HuGene_st:555631- | | | | | |
| | HuGene_st:595266-HuGene_st:503408- | | | | | |
| | HuGene_st:296998-HuGene_st:672458- | | | | | |
| | HuGene_st:64704-HuGene_st:285813- | | | | | |
| | HuGene_st:865055-HuGene_st:734911-HuGene_st | | | | | |
| QKI | 214543_x_at:212262_at:696914-HuGene_st:363144- | 1.93E−05 | 1.3452 | 1.31 | 74.9 | 65.8-82.6 |
| | HuGene_st:820387-HuGene_st:646624- | | | | | |
| | HuGene_st:979853-HuGene_st:658669- | | | | | |
| | HuGene_st:748520-HuGene_st:656477- | | | | | |
| | HuGene_st:296134- | | | | | |
| | HuGene_st:1555154_a_at:212636_at:234492_at:462894- | | | | | |
| | HuGene_st:1055543-HuGene_st | | | | | |
| VSIG2 | 985113-HuGene_st:555767-HuGene_st:391438- | 2.01E−05 | 1.2959 | 2.05 | 74.1 | 65.1-81.9 |
| | HuGene_st:1093892-HuGene_st:675062- | | | | | |
| | HuGene_st:788466-HuGene_st:826720- | | | | | |
| | HuGene_st:997596-HuGene_st:1069591- | | | | | |
| | HuGene_st:228232_s_at:223925-HuGene_st:637781- | | | | | |
| | HuGene_st:265221-HuGene_st:652010- | | | | | |
| | HuGene_st:265186-HuGene_st:343187- | | | | | |
| | HuGene_st:1095607-HuGene_st | | | | | |
| HSPA1A | 979726-HuGene_st:1069731-HuGene_st:50191- | 2.16E−05 | 1.2699 | 2.41 | 73.7 | 64.6-81.5 |
| | HuGene_st:799087-HuGene_st:200799_at:539596- | | | | | |
| | HuGene_st:150764-HuGene_st:200800_s_at:492579- | | | | | |
| | HuGene_st | | | | | |
| AP1S2 | 230413_s_at:230264_s_at:99873-HuGene_st:57979- | 3.39E−05 | 1.2572 | 1.92 | 73.5 | 64.3-81.3 |
| | HuGene_st:985324-HuGene_st:203300_x_at:84828- | | | | | |
| | HuGene_st:910415-HuGene_st | | | | | |
| FKBP5 | 204560_at:224856_at:246403-HuGene_st:513199- | 3.41E−05 | 1.2369 | 1.71 | 73.2 | 64-81 |
| | HuGene_st:69253-HuGene_st:642461- | | | | | |
| | HuGene_st:467598-HuGene_st:1038165- | | | | | |
| | HuGene_st:512075-HuGene_st:224840_at:797941- | | | | | |
| | HuGene_st:633285-HuGene_st:511644- | | | | | |
| | HuGene_st:469540-HuGene_st:209829- | | | | | |
| | HuGene_st:800516-HuGene_st:247764- | | | | | |
| | HuGene_st:468711-HuGene_st:112353- | | | | | |
| | HuGene_st:746630-HuGene_st:337035- | | | | | |
| | HuGene_st:1014328-HuGene_st:292916-HuGene_st | | | | | |
| KCNMB1 | 17348-HuGene_st:414025-HuGene_st:792493- | 3.74E−05 | 1.2189 | 2.66 | 72.9 | 63.7-80.8 |
| | HuGene_st:131717-HuGene_st:569127- | | | | | |
| | HuGene_st:17962-HuGene_st:504462- | | | | | |
| | HuGene_st:758432-HuGene_st:209948_at:772102- | | | | | |
| | HuGene_st:518039-HuGene_st | | | | | |
| VIM | 564251-HuGene_st:201426_s_at:234475- | 4.15E−05 | 1.2225 | 1.86 | 72.9 | 63.7-80.9 |
| | HuGene_st:319318-HuGene_st:339807- | | | | | |
| | HuGene_st:527110-HuGene_st:994975- | | | | | |
| | HuGene_st:837477-HuGene_st:302722- | | | | | |
| | HuGene_st:516025-HuGene_st:1079757- | | | | | |
| | HuGene_st:398387-HuGene_st:155888- | | | | | |
| | HuGene_st:139661-HuGene_st:192324- | | | | | |
| | HuGene_st:1093618-HuGene_st:364983- | | | | | |
| | HuGene_st:436158-HuGene_st:410777- | | | | | |
| | HuGene_st:1555938_x_at | | | | | |
| SORBS2 | 238751_at:805920-HuGene_st | 4.23E−05 | 1.3007 | 2.1 | 74.2 | 65.1-82 |
| C6orf204 | 486932-HuGene_st:573436-HuGene_st:400510- | 4.90E−05 | 1.2042 | 2.07 | 72.6 | 63.5-80.6 |
| | HuGene_st:228202_at:123307-HuGene_st:181714- | | | | | |
| | HuGene_st:206020-HuGene_st:949623- | | | | | |
| | HuGene_st:814579-HuGene_st:515524- | | | | | |
| | HuGene_st:514175-HuGene_st | | | | | |
| FAM55D | 633727-HuGene_st:966568-HuGene_st:625133- | 5.67E−05 | 1.1792 | 3.48 | 72.2 | 62.9-80.2 |
| | HuGene_st:858568-HuGene_st:220645_at:201856- | | | | | |
| | HuGene_st:1013940-HuGene_st:316604- | | | | | |
| | HuGene_st:450702-HuGene_st:1028406- | | | | | |
| | HuGene_st:1090862-HuGene_st:741930- | | | | | |
| | HuGene_st:981211-HuGene_st:970069- | | | | | |
| | HuGene_st:259706-HuGene_st:291282- | | | | | |
| | HuGene_st:56493-HuGene_st:888970- | | | | | |
| | HuGene_st:821065-HuGene_st:53354- | | | | | |
| | HuGene_st:752570-HuGene_st:539086- | | | | | |
| | HuGene_st:204142-HuGene_st | | | | | |
| TIMP2 | 1087828-HuGene_st:231579_s_at:436546- | 6.40E−05 | 1.2298 | 1.82 | 73.1 | 63.8-80.9 |
| | HuGene_st:799495-HuGene_st:563754- | | | | | |
| | HuGene_st:914626-HuGene_st:526357- | | | | | |
| | HuGene_st:203167_at:760416-HuGene_st:651774- | | | | | |
| | HuGene_st:965142-HuGene_st:224540- | | | | | |
| | HuGene_st:690107-HuGene_st:224560_at:319958- | | | | | |
| | HuGene_st:437652-HuGene_st:43390- | | | | | |
| | HuGene_st:727465-HuGene_st:310783- | | | | | |
| | HuGene_st:750028-HuGene_st:957648- | | | | | |
| | HuGene_st:106688-HuGene_st:289430- | | | | | |
| | HuGene_st:740790-HuGene_st | | | | | |
| ELOVL5 | NA | 8.15E−05 | 1.1814 | 2.05 | 72.3 | 63-80.3 |
| ACTA2 | 215787_at:743686-HuGene_st:200974_at:1070983- | 1.00E−04 | 1.2318 | 1.81 | 73.1 | 63.9-80.9 |
| | HuGene_st:120087-HuGene_st:980364- | | | | | |
| | HuGene_st:529704-HuGene_st:855050- | | | | | |
| | HuGene_st:530590-HuGene_st:692252- | | | | | |
| | HuGene_st:404109-HuGene_st:630797- | | | | | |
| | HuGene_st:670805-HuGene_st:293590- | | | | | |
| | HuGene_st:757648-HuGene_st:445454- | | | | | |
| | HuGene_st:479447-HuGene_st:548186- | | | | | |
| | HuGene_st:237159-HuGene_st:90357- | | | | | |
| | HuGene_st:313690-HuGene_st:183075- | | | | | |
| | HuGene_st:785232-HuGene_st | | | | | |
| C1S | 208747_s_at:233042_at:620011- | 0.0001 | 1.2896 | 1.61 | 74 | 64.8-81.9 |
| | HuGene_st:1555229_a_at:245571-HuGene_st:426716- | | | | | |
| | HuGene_st:866948-HuGene_st:705447- | | | | | |
| | HuGene_st:947234-HuGene_st:682561- | | | | | |
| | HuGene_st:332123-HuGene_st | | | | | |
| LOC387763 | 1062323-HuGene_st | 0.0001 | 1.1943 | 1.36 | 72.5 | 63.2-80.5 |
| CLU | 222043_at:939366-HuGene_st:832450- | 0.0001 | 1.2285 | 1.42 | 73 | 63.8-81 |
| | HuGene_st:208792_s_at:125360-HuGene_st:216446- | | | | | |
| | HuGene_st:208791_at:1054768-HuGene_st:1034873- | | | | | |
| | HuGene_st:1056536-HuGene_st:787017- | | | | | |
| | HuGene_st:1060096-HuGene_st:591687- | | | | | |
| | HuGene_st:1014914-HuGene_st:213483- | | | | | |
| | HuGene_st:459630-HuGene_st:1036064- | | | | | |
| | HuGene_st:972503-HuGene_st:492827- | | | | | |
| | HuGene_st:485686-HuGene_st | | | | | |
| MT1H | 197989-HuGene_st:589780-HuGene_st:800631- | 0.0001 | 1.1885 | 4.55 | 72.4 | 63.1-80.4 |
| | HuGene_st:854292-HuGene_st:644419- | | | | | |
| | HuGene_st:851562-HuGene_st:590830- | | | | | |
| | HuGene_st:727551-HuGene_st:51225- | | | | | |
| | HuGene_st:206461_x_at:1099945-HuGene_st:334778- | | | | | |
| | HuGene_st:6545-HuGene_st:451479- | | | | | |
| | HuGene_st:621584-HuGene_st:885933- | | | | | |
| | HuGene_st:73199-HuGene_st:882631-HuGene_st | | | | | |
| RGS2 | 331021-HuGene_st:202388_at:895469- | 0.0001 | 1.1617 | 1.94 | 71.9 | 62.6-79.9 |
| | HuGene_st:962521-HuGene_st:843420- | | | | | |
| | HuGene_st:767847-HuGene_st:1022759- | | | | | |
| | HuGene_st:507303-HuGene_st:121934- | | | | | |
| | HuGene_st:1094262-HuGene_st:1067241- | | | | | |
| | HuGene_st:111860-HuGene_st:661918-HuGene_st | | | | | |
| FBN1 | 192231-HuGene_st:794561-HuGene_st:1094040- | 0.0002 | 1.1368 | 1.93 | 71.5 | 62.2-79.6 |
| | HuGene_st:427611-HuGene_st:394963- | | | | | |
| | HuGene_st:828273-HuGene_st:202765_s_at:162818- | | | | | |
| | HuGene_st:169932-HuGene_st:192029- | | | | | |
| | HuGene_st:288955-HuGene_st:600623- | | | | | |
| | HuGene_st:941525-HuGene_st:971683- | | | | | |
| | HuGene_st:82155-HuGene_st:359130- | | | | | |
| | HuGene_st:404656-HuGene_st:921008- | | | | | |
| | HuGene_st:999573-HuGene_st:112593-HuGene_st | | | | | |
| ANKRD25 | 808074-HuGene_st:932564-HuGene_st:143731- | 0.0002 | 1.1162 | 1.56 | 71.2 | 61.9-79.3 |
| | HuGene_st:292383-HuGene_st:393569- | | | | | |
| | HuGene_st:201770-HuGene_st:973796- | | | | | |
| | HuGene_st:824703-HuGene_st:109143- | | | | | |
| | HuGene_st:132685-HuGene_st:698165- | | | | | |
| | HuGene_st:218418_s_at | | | | | |
| MT1G | 226585-HuGene_st:495514-HuGene_st:159768- | 0.0002 | 1.0893 | 2.66 | 70.7 | 61.3-78.8 |
| | HuGene_st:186270-HuGene_st:855342- | | | | | |
| | HuGene_st:449379-HuGene_st:1100995- | | | | | |
| | HuGene_st:617733-HuGene_st:914660- | | | | | |
| | HuGene_st:884883-HuGene_st:903179- | | | | | |
| | HuGene_st:311881-HuGene_st:977536- | | | | | |
| | HuGene_st:210472_at:1061010-HuGene_st:415267- | | | | | |
| | HuGene_st:1048754-HuGene_st:1011704- | | | | | |
| | HuGene_st:59546-HuGene_st:917923- | | | | | |
| | HuGene_st:204745_x_at | | | | | |
| MYL9 | 201058_s_at:543240-HuGene_st:923797- | 0.0003 | 1.1288 | 2.18 | 71.4 | 62-79.4 |
| | HuGene_st:173220-HuGene_st:874422- | | | | | |
| | HuGene_st:925187-HuGene_st:979054- | | | | | |
| | HuGene_st:676360-HuGene_st:117460- | | | | | |
| | HuGene_st:1092848-HuGene_st:608837- | | | | | |
| | HuGene_st:404703-HuGene_st:50995- | | | | | |
| | HuGene_st:206106-HuGene_st:995028-HuGene_st | | | | | |
| TAGLN | 279405-HuGene_st:205547_s_at:695669- | 0.0003 | 1.0901 | 2.12 | 70.7 | 61.4-78.8 |
| | HuGene_st:902958-HuGene_st:304704- | | | | | |
| | HuGene_st:741240-HuGene_st:898711- | | | | | |
| | HuGene_st:1555724_s_at:26929-HuGene_st:66114- | | | | | |
| | HuGene_st:505391-HuGene_st:613243- | | | | | |
| | HuGene_st:73428-HuGene_st:983072- | | | | | |
| | HuGene_st:543865-HuGene_st:179049- | | | | | |
| | HuGene_st:458482-HuGene_st:823959- | | | | | |
| | HuGene_st:931316-HuGene_st:556028- | | | | | |
| | HuGene_st:565900-HuGene_st:278667- | | | | | |
| | HuGene_st:226523_at:232248_at | | | | | |
| FOXF1 | 21663-HuGene_st:832719-HuGene_st:174216- | 0.0004 | 1.1745 | 1.28 | 72.1 | 62.9-80.1 |
| | HuGene_st:337755-HuGene_st:775422- | | | | | |
| | HuGene_st:205935_at | | | | | |
| LGALS2 | 204754-HuGene_st:455546-HuGene_st:428640- | 0.0004 | 1.0851 | 3.07 | 70.6 | 61.2-78.8 |
| | HuGene_st:228729-HuGene_st:450048- | | | | | |
| | HuGene_st:1087923-HuGene_st:616130- | | | | | |
| | HuGene_st:818575-HuGene_st:699850- | | | | | |
| | HuGene_st:820908-HuGene_st:850596- | | | | | |
| | HuGene_st:751645-HuGene_st:1028381- | | | | | |
| | HuGene_st:667147-HuGene_st:245361- | | | | | |
| | HuGene_st:754620-HuGene_st | | | | | |
| SDC2 | 1019857-HuGene_st:730583-HuGene_st:33355- | 0.0006 | 1.099 | 1.39 | 70.9 | 61.5-79 |
| | HuGene_st:717018-HuGene_st:212158_at:272847- | | | | | |
| | HuGene_st:458585-HuGene_st:212154_at:446134- | | | | | |
| | HuGene_st:1096317-HuGene_st:815650- | | | | | |
| | HuGene_st:590116-HuGene_st:367740- | | | | | |
| | HuGene_st:1074315-HuGene_st | | | | | |
| DNASE1L3 | 867148-HuGene_st:588394- | 0.0007 | 1.0972 | 1.5 | 70.8 | 61.6-79 |
| | HuGene_st:205554_s_at:616846-HuGene_st:58544- | | | | | |
| | HuGene_st:277847-HuGene_st:18171- | | | | | |
| | HuGene_st:429485-HuGene_st:960168- | | | | | |
| | HuGene_st:981221-HuGene_st:23306- | | | | | |
| | HuGene_st:991254-HuGene_st | | | | | |
| PLEKHC1 | 831786-HuGene_st:88016-HuGene_st:1087035- | 0.0007 | 1.0318 | 1.82 | 69.7 | 60.3-78 |
| | HuGene_st:530331-HuGene_st:209209_s_at:184398- | | | | | |
| | HuGene_st:904923-HuGene_st:193550- | | | | | |
| | HuGene_st:468805-HuGene_st:104495- | | | | | |
| | HuGene_st:604317-HuGene_st:501652- | | | | | |
| | HuGene_st:118096-HuGene_st:321532- | | | | | |
| | HuGene_st:675257-HuGene_st:79496- | | | | | |
| | HuGene_st:590867-HuGene_st:147248- | | | | | |
| | HuGene_st:627402-HuGene_st:377514-HuGene_st | | | | | |
| SERPINF1 | 546682-HuGene_st:814007- | 0.0009 | 1.0012 | 1.47 | 69.2 | 59.8-77.4 |
| | HuGene_st:202283_at:898654-HuGene_st:884794- | | | | | |
| | HuGene_st:264195-HuGene_st:787378- | | | | | |
| | HuGene_st:1036417-HuGene_st | | | | | |
| RARRES2 | 1098138-HuGene_st:515302-HuGene_st:849018- | 0.0009 | 1.0325 | 1.6 | 69.7 | 60.3-78 |
| | HuGene_st:209496_at:741143-HuGene_st:903829- | | | | | |
| | HuGene_st:305131-HuGene_st:715557- | | | | | |
| | HuGene_st:373579-HuGene_st:177871- | | | | | |
| | HuGene_st:106318-HuGene_st:662795- | | | | | |
| | HuGene_st:38090-HuGene_st | | | | | |
| CFL2 | 545956-HuGene_st:833414-HuGene_st:214239- | 0.0009 | 1.0592 | 1.75 | 70.2 | 60.8-78.4 |
| | HuGene_st:192253-HuGene_st:224663_s_at:694174- | | | | | |
| | HuGene_st:233496_s_at:992798-HuGene_st:328584- | | | | | |
| | HuGene_st:950857-HuGene_st:612593- | | | | | |
| | HuGene_st:95686-HuGene_st:177218- | | | | | |
| | HuGene_st:451953-HuGene_st:800232- | | | | | |
| | HuGene_st:575557-HuGene_st:794701-HuGene_st | | | | | |
| RBMS1 | 375424-HuGene_st:87825-HuGene_st:1046943- | 0.0009 | 1.1417 | 1.55 | 71.6 | 62.3-79.7 |
| | HuGene_st:209868_s_at:207266_x_at:225269_s_at:238185_at | | | | | |
| SELENBP1 | 914892-HuGene_st:582091-HuGene_st:643114- | 0.0009 | 1.0563 | 1.7 | 70.1 | 60.7-78.4 |
| | HuGene_st:532506-HuGene_st:823159- | | | | | |
| | HuGene_st:171447-HuGene_st:189960- | | | | | |
| | HuGene_st:75763-HuGene_st:834358- | | | | | |
| | HuGene_st:245623- | | | | | |
| | HuGene_st:214433_s_at:233267_at:564186- | | | | | |
| | HuGene_st:637748-HuGene_st:200643- | | | | | |
| | HuGene_st:781415-HuGene_st:208204- | | | | | |
| | HuGene_st:730504-HuGene_st:1008117- | | | | | |
| | HuGene_st:801694-HuGene_st:433986- | | | | | |
| | HuGene_st:183676-HuGene_st:634656- | | | | | |
| | HuGene_st:1087258-HuGene_st | | | | | |
| ENAM | 212592_at | 0.0011 | 1.0044 | 2.58 | 69.2 | 59.9-77.5 |
| MT1F | 987574-HuGene_st:213629_x_at:171840- | 0.0012 | 0.9654 | 2.2 | 68.5 | 59.1-76.9 |
| | HuGene_st:217165_x_at:867979-HuGene_st:354284- | | | | | |
| | HuGene_st:424055-HuGene_st:998184- | | | | | |
| | HuGene_st:616693-HuGene_st:459005- | | | | | |
| | HuGene_st:221141-HuGene_st:338518- | | | | | |
| | HuGene_st:951452-HuGene_st:915306- | | | | | |
| | HuGene_st:506126-HuGene_st:845635- | | | | | |
| | HuGene_st:1048483-HuGene_st:1094672- | | | | | |
| | HuGene_st:576368-HuGene_st:414800-HuGene_st | | | | | |
| CD163 | 620892-HuGene_st:215049_x_at:32161- | 0.0013 | 1.0234 | 1.9 | 69.6 | 60.2-77.9 |
| | HuGene_st:867013-HuGene_st:899841- | | | | | |
| | HuGene_st:662901-HuGene_st:97726- | | | | | |
| | HuGene_st:180883-HuGene_st:706228- | | | | | |
| | HuGene_st:849505-HuGene_st:217692- | | | | | |
| | HuGene_st:566183-HuGene_st:154492- | | | | | |
| | HuGene_st:737668-HuGene_st:203645_s_at:948100- | | | | | |
| | HuGene_st:900552-HuGene_st:288236:HuGene_st | | | | | |
| GUCY1A3 | 560981-HuGene_st:1088572-HuGene_st:692186- | 0.0015 | 1.0136 | 2.69 | 69.4 | 60-77.7 |
| | HuGene_st:648114-HuGene_st:783434- | | | | | |
| | HuGene_st:221942_s_at:1026405-HuGene_st:256104- | | | | | |
| | HuGene_st:965063-HuGene_st:889044- | | | | | |
| | HuGene_st:752296-HuGene_st:239580_at:185668- | | | | | |
| | HuGene_st:120195-HuGene_st:324658- | | | | | |
| | HuGene_st:229530_at | | | | | |
| TNFRSF17 | 1062688-HuGene_st:38682-HuGene_st:558270- | 0.0015 | 1.0199 | 2.08 | 69.5 | 60.1-77.7 |
| | HuGene_st:631025-HuGene_st:24518- | | | | | |
| | HuGene_st:240754-HuGene_st:193693- | | | | | |
| | HuGene_st:255074-HuGene_st:493368- | | | | | |
| | HuGene_st:191710-HuGene_st:610232- | | | | | |
| | HuGene_st:544680-HuGene_st:205576- | | | | | |
| | HuGene_st:738267-HuGene_st:746498-HuGene_st | | | | | |
| CAV1 | 793412-HuGene_st:1007317- | 0.0016 | 1.0215 | 1.58 | 69.5 | 60.1-77.8 |
| | HuGene_st:212097_at:203065_s_at:812832- | | | | | |
| | HuGene_st:505037-HuGene_st:819580- | | | | | |
| | HuGene_st:348883-HuGene_st:659416- | | | | | |
| | HuGene_st:698493-HuGene_st:861877- | | | | | |
| | HuGene_st:518081-HuGene_st:35773-HuGene_st | | | | | |
| CKB | 718720-HuGene_st:769422-HuGene_st:581051- | 0.0019 | 0.9741 | 2.27 | 68.7 | 59.2-77 |
| | HuGene_st:549987-HuGene_st:24890- | | | | | |
| | HuGene_st:1041238-HuGene_st:16603- | | | | | |
| | HuGene_st:480007-HuGene_st:435665- | | | | | |
| | HuGene_st:25470-HuGene_st:40899- | | | | | |
| | HuGene_st:896708-HuGene_st:373774- | | | | | |
| | HuGene_st:963331-HuGene_st:23457- | | | | | |
| | HuGene_st:766369-HuGene_st:200884_at:405417- | | | | | |
| | HuGene_st:888623-HuGene_st:396007-HuGene_st | | | | | |
| WWTR1 | 202132_at:249804-HuGene_st:735408- | 0.002 | 0.9904 | 1.48 | 69 | 59.6-77.3 |
| | HuGene_st:925151-HuGene_st:213564- | | | | | |
| | HuGene_st:561456-HuGene_st:125734- | | | | | |
| | HuGene_st:367743-HuGene_st:153369- | | | | | |
| | HuGene_st:628871-HuGene_st:942493- | | | | | |
| | HuGene_st:949013-HuGene_st:202134_s_at:426662- | | | | | |
| | HuGene_st:586249-HuGene_st:228629_s_at:506281- | | | | | |
| | HuGene_st | | | | | |
| HMGCS2 | 729816-HuGene_st:658722-HuGene_st:253113- | 0.0022 | 0.8767 | 2.67 | 66.9 | 57.5-75.5 |
| | HuGene_st:900260-HuGene_st:253100- | | | | | |
| | HuGene_st:283650-HuGene_st:624250- | | | | | |
| | HuGene_st:439478-HuGene_st:407398- | | | | | |
| | HuGene_st:171889-HuGene_st:397691- | | | | | |
| | HuGene_st:448587-HuGene_st:593619- | | | | | |
| | HuGene_st:371076-HuGene_st:228587- | | | | | |
| | HuGene_st:888308-HuGene_st:538023- | | | | | |
| | HuGene_st:204607_at:43162-HuGene_st:865416- | | | | | |
| | HuGene_st:619083-HuGene_st:789982- | | | | | |
| | HuGene_st:616916-HuGene_st | | | | | |
| MAFB | 876871-HuGene_st:600037-HuGene_st:177002- | 0.0034 | 0.9563 | 1.66 | 68.4 | 59-76.8 |
| | HuGene_st:3125-HuGene_st:1057503- | | | | | |
| | HuGene_st:274807-HuGene_st | | | | | |
| STOM | 201060_x_at:669723-HuGene_st:1099166- | 0.0035 | 1.0176 | 1.21 | 69.5 | 60.1-77.7 |
| | HuGene_st:201061_s_at:22513-HuGene_st:430393- | | | | | |
| | HuGene_st:486266-HuGene_st:47888-HuGene_st | | | | | |
| CD14 | 327635-HuGene_st:201743_at:822666- | 0.0035 | 0.9646 | 1.58 | 68.5 | 59-76.9 |
| | HuGene_st:999890-HuGene_st:753790- | | | | | |
| | HuGene_st:1080129-HuGene_st:532035- | | | | | |
| | HuGene_st:1087885-HuGene_st:528527- | | | | | |
| | HuGene_st:851691-HuGene_st:905038- | | | | | |
| | HuGene_st:437599-HuGene_st:31858-HuGene_st | | | | | |
| MUC4 | 1061780-HuGene_st:43545- | 0.0039 | 1.0448 | 1.2 | 69.9 | 60.5-78.2 |
| | HuGene_st:217110_s_at:47402-HuGene_st:1042572- | | | | | |
| | HuGene_st:548777-HuGene_st:612341- | | | | | |
| | HuGene_st:711231-HuGene_st:217109_at:278229- | | | | | |
| | HuGene_st:536580-HuGene_st:235055_x_at:743233- | | | | | |
| | HuGene_st:223515-HuGene_st:204895_x_at:670488- | | | | | |
| | HuGene_st:106413-HuGene_st:35359- | | | | | |
| | HuGene_st:344313-HuGene_st:284609- | | | | | |
| | HuGene_st:882714-HuGene_st:512794-HuGene_st | | | | | |
| APOE | 520118-HuGene_st:886930-HuGene_st:771679- | 0.0044 | 0.9406 | 1.56 | 68.1 | 58.7-76.6 |
| | HuGene_st:944600-HuGene_st:169546- | | | | | |
| | HuGene_st:483757-HuGene_st:16650- | | | | | |
| | HuGene_st:511913-HuGene_st:1099818- | | | | | |
| | HuGene_st:33219-HuGene_st:663513-HuGene_st | | | | | |
| SI | 9605-HuGene_st:514814-HuGene_st:809314- | 0.0054 | 0.9299 | 2.79 | 67.9 | 58.5-76.4 |
| | HuGene_st:576613-HuGene_st:45502- | | | | | |
| | HuGene_st:369676-HuGene_st:438636- | | | | | |
| | HuGene_st:267204-HuGene_st:326716- | | | | | |
| | HuGene_st:897825-HuGene_st:377666- | | | | | |
| | HuGene_st:519273-HuGene_st:325741- | | | | | |
| | HuGene_st:245381-HuGene_st:108368- | | | | | |
| | HuGene_st:464198-HuGene_st:679193- | | | | | |
| | HuGene_st:168180-HuGene_st | | | | | |
| LOC285382 | 242447_at | 0.0058 | 0.7422 | 1.26 | 64.5 | 55-73.2 |
| HLA- | 971813-HuGene_st | 0.0063 | 1.0668 | 1.51 | 70.3 | 60.9-78.6 |
| DRB1 | | | | | | |
| ANTXR1 | 241549_at:232956_at | 0.0071 | 0.9507 | 1.29 | 68.3 | 58.8-76.7 |
| FAM46C | 197110-HuGene_st:220306_at:110466- | 0.0085 | 0.9172 | 1.38 | 67.7 | 58.2-76.1 |
| | HuGene_st:988119-HuGene_st:1098424- | | | | | |
| | HuGene_st:560529-HuGene_st:387944- | | | | | |
| | HuGene_st:38645-HuGene_st:221689- | | | | | |
| | HuGene_st:322082-HuGene_st | | | | | |
| TIMP3 | 767021-HuGene_st:201150_s_at:201148_s_at:228752- | 0.0091 | 0.9064 | 1.81 | 67.5 | 58-76 |
| | HuGene_st:241221-HuGene_st:201147_s_at:125730- | | | | | |
| | HuGene_st | | | | | |
| LOC283666 | 235567_at:236266_at:1562682_at:240951_at | 0.0099 | 0.7796 | 1.22 | 65.2 | 55.6-73.8 |
| GREM1 | 569365-HuGene_st:407017- | 0.0106 | 0.8579 | 3.03 | 66.6 | 57-75.1 |
| | HuGene_st:218468_s_at:292333-HuGene_st:330742- | | | | | |
| | HuGene_st:546941-HuGene_st:556201- | | | | | |
| | HuGene_st:484435-HuGene_st:326978- | | | | | |
| | HuGene_st:144073-HuGene_st:411208- | | | | | |
| | HuGene_st:1081939-HuGene_st:345681- | | | | | |
| | HuGene_st:2445-HuGene_st:864620-HuGene_st | | | | | |
| FCGBP | 22847-HuGene_st:203240_at:338164- | 0.0121 | 0.7889 | 1.99 | 65.3 | 55.7-74 |
| | HuGene_st:516715-HuGene_st:88809- | | | | | |
| | HuGene_st:948682-HuGene_st:311904- | | | | | |
| | HuGene_st:997435-HuGene_st:841000- | | | | | |
| | HuGene_st:508230-HuGene_st:226428- | | | | | |
| | HuGene_st:634071-HuGene_st:948120- | | | | | |
| | HuGene_st:1079875-HuGene_st:71797- | | | | | |
| | HuGene_st:781083-HuGene_st:426005- | | | | | |
| | HuGene_st:27264-HuGene_st:16108- | | | | | |
| | HuGene_st:63672-HuGene_st:1085208- | | | | | |
| | HuGene_st:708701-HuGene_st | | | | | |
| HSPA2 | 831351-HuGene_st:270747-HuGene_st:720913- | 0.0123 | 0.8908 | 1.43 | 67.2 | 57.7-75.7 |
| | HuGene_st:660812-HuGene_st:939000- | | | | | |
| | HuGene_st:920196-HuGene_st | | | | | |
| COL6A1 | 138366-HuGene_st:528191-HuGene_st:884210- | 0.0126 | 0.8452 | 1.45 | 66.4 | 56.9-75 |
| | HuGene_st:57954-HuGene_st:737256- | | | | | |
| | HuGene_st:858575- | | | | | |
| | HuGene_st:212937_s_at:214200_s_at:190064- | | | | | |
| | HuGene_st:216904_at | | | | | |
| SELM | NA | 0.0133 | 0.8596 | 1.32 | 66.6 | 57.2-75.2 |
| FLNA | 296693-HuGene_st:685285-HuGene_st:704065- | 0.0157 | 0.7967 | 1.59 | 65.5 | 55.9-74.1 |
| | HuGene_st:445061-HuGene_st:11735- | | | | | |
| | HuGene_st:1066792- | | | | | |
| | HuGene_st:214752_x_at:213746_s_at:871038- | | | | | |
| | HuGene_st:396236-HuGene_st:200859_x_at:786282- | | | | | |
| | HuGene_st:136680-HuGene_st:50466- | | | | | |
| | HuGene_st:955232-HuGene_st:588605- | | | | | |
| | HuGene_st:40025-HuGene_st:1002306- | | | | | |
| | HuGene_st:833338-HuGene_st:86435- | | | | | |
| | HuGene_st:172457-HuGene_st | | | | | |
| COX7A1 | 386303-HuGene_st:354428-HuGene_st | 0.0161 | 0.8613 | 1.32 | 66.7 | 57.2-75.3 |
| EMP3 | 736604-HuGene_st:155271-HuGene_st:584704- | 0.0164 | 0.8053 | 1.41 | 65.6 | 56.1-74.2 |
| | HuGene_st | | | | | |
| ITLN1 | 389198-HuGene_st:496499-HuGene_st:48547- | 0.021 | 0.7137 | 2.54 | 63.9 | 54.4-72.7 |
| | HuGene_st:552029-HuGene_st:126777- | | | | | |
| | HuGene_st:223597_at:281671-HuGene_st:45052- | | | | | |
| | HuGene_st:573120:HuGene_st:137624- | | | | | |
| | HuGene_st:948945-HuGene_st:527196- | | | | | |
| | HuGene_st:358648-HuGene_st:45640- | | | | | |
| | HuGene_st:512550-HuGene_st:839317- | | | | | |
| | HuGene_st:634036-HuGene_st:101083- | | | | | |
| | HuGene_st:857541-HuGene_st:404069-HuGene_st | | | | | |
| C10orf99 | 227735_s_at:563767-HuGene_st:963471- | 0.0218 | 0.724 | 1.76 | 64.1 | 54.7-72.9 |
| | HuGene_st:965947-HuGene_st:1081062- | | | | | |
| | HuGene_st:354432-HuGene_st:602661- | | | | | |
| | HuGene_st:227736_at:1009594-HuGene_st:969063- | | | | | |
| | HuGene_st:77052-HuGene_st:483367- | | | | | |
| | HuGene_st:50662-HuGene_st:738457- | | | | | |
| | HuGene_st:69019-HuGene_st:215415- | | | | | |
| | HuGene_st:1003926-HuGene_st:676577- | | | | | |
| | HuGene_st:676855-HuGene_st:225297- | | | | | |
| | HuGene_st:560897-HuGene_st:886384- | | | | | |
| | HuGene_st:303754-HuGene_st | | | | | |
| CSRP1 | 453033-HuGene_st:673637-HuGene_st:607761- | 0.023 | 0.7234 | 1.39 | 64.1 | 54.6-72.9 |
| | HuGene_st:582630-HuGene_st:654799- | | | | | |
| | HuGene_st:258474-HuGene_st:200621_at:1028504- | | | | | |
| | HuGene_st:802909-HuGene_st:339914- | | | | | |
| | HuGene_st:533385-HuGene_st:256461- | | | | | |
| | HuGene_st:1043113-HuGene_st:72101- | | | | | |
| | HuGene_st:105671-HuGene_st:339691- | | | | | |
| | HuGene_st:108781-HuGene_st:133369- | | | | | |
| | HuGene_st:614016-HuGene_st:629648- | | | | | |
| | HuGene_st:81550-HuGene_st | | | | | |
| PTGER4 | 450915-HuGene_st:1067155-HuGene_st:812085- | 0.0237 | 0.8101 | 1.41 | 65.7 | 56.2-74.4 |
| | HuGene_st:1943-HuGene_st:109314- | | | | | |
| | HuGene_st:204897_at:252442-HuGene_st:800125- | | | | | |
| | HuGene_st:965220:HuGene_st:446764- | | | | | |
| | HuGene_st:204896_s_at:23686-HuGene_st:548266- | | | | | |
| | HuGene_st:392964:HuGene_st:986807- | | | | | |
| | HuGene_st:619166-HuGene_st | | | | | |
| TPM2 | 828216-HuGene_st:373854-HuGene_st:981165- | 0.026 | 0.7539 | 1.53 | 64.7 | 55.2-73.4 |
| | HuGene_st:191586-HuGene_st:445912- | | | | | |
| | HuGene_st:446500-HuGene_st:922931- | | | | | |
| | HuGene_st:336603-HuGene_st:272114- | | | | | |
| | HuGene_st:375166-HuGene_st:8388- | | | | | |
| | HuGene_st:204083_s_at:544548-HuGene_st:945443- | | | | | |
| | HuGene_st:628440-HuGene_st:763118- | | | | | |
| | HuGene_st:538951-HuGene_st | | | | | |
| PIGR | 748831-HuGene_st:226147_s_at:166398- | 0.0278 | 0.7282 | 1.59 | 64.2 | 54.7-73 |
| | HuGene_st:593192-HuGene_st:204213_at:557727- | | | | | |
| | HuGene_st:229659_s_at:242106-HuGene_st:146237- | | | | | |
| | HuGene_st:883137-HuGene_st:691362- | | | | | |
| | HuGene_st:201991-HuGene_st:1045753- | | | | | |
| | HuGene_st:589879-HuGene_st:1017162- | | | | | |
| | HuGene_st:325979-HuGene_st:105903- | | | | | |
| | HuGene_st:1063932-HuGene_st:207526- | | | | | |
| | HuGene_st:566905-HuGene_st | | | | | |
| HLA-C | 754900-HuGene_st | 0.0279 | 0.9253 | 1.24 | 67.8 | 58.3-76.2 |
| PRNP | 178399-HuGene_st:201300_s_at:386578- | 0.0288 | 0.798 | 1.24 | 65.5 | 56-74.2 |
| | HuGene_st:783439-HuGene_st | | | | | |
| CCL11 | 495934-HuGene_st | 0.0361 | 0.8547 | 1.32 | 66.5 | 57.1-75 |
| C1QC | 137801-HuGene_st:851445-HuGene_st | 0.0391 | 0.7457 | 1.36 | 64.5 | 54.9-73.3 |
| CYR61 | 736150-HuGene_st:385534-HuGene_st:48239- | 0.0425 | 0.7875 | 1.47 | 65.3 | 55.7-73.9 |
| | HuGene_st:242630-HuGene_st | | | | | |
| SERPING1 | NA | 0.0503 | 0.7659 | 1.44 | 64.9 | 55.4-73.6 |
| TYROBP | NA | 0.053 | 0.8176 | 1.17 | 65.9 | 56.3-74.5 |
| GJA1 | NA | 0.0561 | 0.7839 | 1.76 | 65.2 | 55.6-73.9 |
| PTRF | NA | 0.0624 | 0.7354 | 1.5 | 64.3 | 54.8-73.1 |
| CLCA1 | NA | 0.0681 | 0.7855 | 1.61 | 65.3 | 55.8-74 |
| SPARC | NA | 0.0684 | 0.6702 | 1.45 | 63.1 | 53.5-71.9 |
| COL4A2 | NA | 0.0738 | 0.7081 | 1.32 | 63.8 | 54.3-72.5 |
| RAB31 | NA | 0.1041 | 0.8159 | 1.14 | 65.8 | 56.3-74.4 |
| NID1 | NA | 0.105 | 0.7428 | 1.33 | 64.5 | 54.9-73.2 |
| FN1 | NA | 0.1121 | 0.5769 | 1.73 | 61.3 | 51.7-70.2 |
| MSN | NA | 0.1499 | 0.7215 | 1.15 | 64.1 | 54.6-72.9 |
| MMP2 | NA | 0.1755 | 0.6219 | 1.24 | 62.2 | 52.5-71.2 |
| COL1A2 | NA | 0.1891 | 0.65 | 1.9 | 62.7 | 53.2-71.6 |
| FOSB | NA | 0.2076 | 0.6389 | 2.17 | 62.5 | 52.9-71.4 |
| HBB | NA | 0.2357 | 0.6434 | 1.28 | 62.6 | 53-71.5 |
| POU2AF1 | NA | 0.2389 | 0.6564 | 1.18 | 62.9 | 53.3-71.7 |
| COL3A1 | NA | 0.2551 | 0.5538 | 1.35 | 60.9 | 51.3-69.9 |
| SMOC2 | NA | 0.2701 | 0.6533 | 1.34 | 62.8 | 53.2-71.7 |
| MUC2 | NA | 0.2802 | 0.8523 | 1.22 | 66.5 | 57-75 |
| Sep-06 | NA | 0.3109 | 0.5713 | 1.21 | 61.2 | 51.7-70.2 |
| LUM | NA | 0.3157 | 0.5442 | 1.54 | 60.7 | 51.1-69.7 |
| C1QB | NA | 0.3668 | 0.5005 | 1.24 | 59.9 | 50.4-69 |
| TGFB1I1 | NA | 0.4093 | 0.4951 | 1.35 | 59.8 | 50.1-68.8 |
| ALDH1A1 | NA | 0.4232 | 0.354 | 1.4 | 57 | 47.4-66.2 |
| LRIG1 | NA | 0.4614 | 0.5998 | 1.26 | 61.8 | 52.2-70.7 |
| C8orf4 | NA | 0.475 | 0.483 | 1.44 | 59.5 | 49.9-68.5 |
| EGR1 | NA | 0.4851 | 0.5374 | 1.38 | 60.6 | 51-69.6 |
| LOC652745 | NA | 0.4864 | 0.1722 | 1.09 | 53.4 | 43.8-62.8 |
| IGKV1D- | NA | 0.5177 | 0.1597 | 1.1 | 53.2 | 43.7-62.5 |
| 13 | | | | | | |
| COL15A1 | NA | 0.6601 | 0.54 | 1.22 | 60.6 | 51-69.6 |
| IGFBP5 | NA | 0.7663 | 0.3504 | 1.25 | 57 | 47.4-66.3 |
| LGALS1 | NA | 0.8555 | 0.4626 | 1.16 | 59.1 | 49.5-68.3 |
| COL6A3 | NA | 0.8739 | 0.4412 | 1.2 | 58.7 | 49.1-67.9 |
| HLA- | NA | 0.9084 | 0.5417 | 1.28 | 60.7 | 51.1-69.6 |
| DQA1 | | | | | | |
| HLA-DRA | NA | 0.9084 | 0.5391 | 1.28 | 60.6 | 51-69.6 |
| ST6GALNAC1 | NA | 0.9452 | 0.2594 | 1.15 | 55.2 | 45.6-64.5 |
| HLA-DPA1 | NA | 0.9664 | 0.4166 | 1.39 | 58.3 | 48.7-67.4 |
| IGLV1-44 | NA | 0.9777 | 0.2865 | 1.07 | 55.7 | 46.1-64.9 |
| TNC | NA | 0.9905 | 0.3682 | 1.09 | 57.3 | 47.7-66.5 |
| HBA1 | NA | 0.9979 | 0.47 | 1.21 | 59.3 | 49.6-68.4 |
| HBA2 | NA | 0.9979 | 0.4705 | 1.21 | 59.3 | 49.6-68.3 |

TABLE 3

| | | Signif. | | | Sens- | |
|---|---|---|---|---|---|---|
| TargetPS | Symbol | FDR | D.val5 | FC | Spec | CI (95) |
| 230788_at | SPTLC3: GCNT2 | 2.1587E−27 | 3.9562 | 13.36 | 97.6 | 94.2-99.2 |
| 228706_s_at | CLDN23 | 4.2694E−20 | 3.0727 | 3.47 | 93.8 | 88.3-97.1 |
| 231120_x_at | PKIB | 5.9397E−20 | 3.1191 | 3.28 | 94.1 | 88.6-97.3 |
| 224412_s_at | TRPM6 | 2.2514E−19 | 3.0115 | 7.5 | 93.4 | 87.7-96.8 |
| 220037_s_at | XLKD1 | 3.5991E−19 | 2.9405 | 7.44 | 92.9 | 87-96.5 |
| 209613_s_at | ADH1B: ADH1A | 4.6957E−19 | 3.0158 | 4.67 | 93.4 | 87.7-96.9 |
| 204719_at | ABCA8 | 1.2559E−18 | 2.9171 | 6.82 | 92.8 | 86.8-96.5 |
| 231773_at | ANGPTL1 | 1.4172E−17 | 2.7946 | 4.64 | 91.9 | 85.6-95.9 |
| 225575_at | LIFR | 2.4532E−17 | 2.7797 | 6.36 | 91.8 | 85.5-95.8 |
| 220812_s_at | HHLA2 | 3.4739E−17 | 2.6718 | 10.02 | 90.9 | 84.4-95.2 |
| 211549_s_at | HPGD | 3.6502E−17 | 2.8451 | 3.21 | 92.3 | 86.1-96.1 |
| 202920_at | ANK2 | 2.5934E−16 | 2.6488 | 6.76 | 90.7 | 84.2-95.1 |
| 231925_at | P2RY1 | 1.4091E−15 | 2.5329 | 3.6 | 89.7 | 82.8-94.4 |
| 207080_s_at | PYY | 3.5022E−15 | 2.4783 | 11.71 | 89.2 | 82.2-94 |
| 228885_at | RPL24: LOC731365 | 1.2312E−14 | 2.5575 | 1.63 | 90 | 83.1-94.6 |
| 228766_at | CD36 | 6.2677E−14 | 2.4687 | 1.65 | 89.1 | 82.1-94 |
| 204931_at | TCF21 | 2.5147E−13 | 2.3036 | 2.34 | 87.5 | 80.1-92.8 |
| 205433_at | BCHE | 3.1756E−13 | 2.2538 | 6.21 | 87 | 79.5-92.5 |
| 207980_s_at | CITED2 | 5.2905E−13 | 2.2709 | 1.64 | 87.2 | 79.7-92.6 |
| 209170_s_at | GPM6B | 4.0429E−12 | 2.1588 | 3.59 | 86 | 78.1-91.6 |
| 220376_at | LRRC19 | 6.7548E−12 | 2.0763 | 4.51 | 85 | 77.1-90.9 |
| 228504_at | No Symbol | 9.1926E−12 | 2.152 | 1.61 | 85.9 | 78.2-91.6 |
| 228854_at | No Symbol | 9.1926E−12 | 2.1538 | 1.61 | 85.9 | 78.2-91.6 |
| 235146_at | No Symbol | 9.1926E−12 | 2.1537 | 1.61 | 85.9 | 78.2-91.6 |
| 221305_s_at | UGT1A10: UGT1A7: UGT1A8: | 1.0319E−11 | 2.0677 | 4.56 | 84.9 | 77.1-90.9 |
| | UGT1A1: UGT1A9: UGT1A6: | | | | | |
| | UGT1A5: UGT1A3: UGT1A4 | | | | | |
| 203881_s_at | DMD | 5.0063E−11 | 2.0137 | 4.74 | 84.3 | 76.3-90.4 |
| 206637_at | P2RY14 | 1.3104E−10 | 1.9836 | 2 | 83.9 | 75.8-90.1 |
| 214598_at | CLDN8 | 2.9225E−10 | 1.8624 | 12.96 | 82.4 | 74.1-88.9 |
| 227529_s_at | AKAP12 | 7.4077E−09 | 1.7434 | 2.83 | 80.8 | 72.3-87.5 |
| 219948_x_at | LOC642329: UGT2A3 | 8.638E−09 | 1.741 | 4.89 | 80.8 | 72.3-87.6 |
| 222717_at | SDPR | 9.6207E−08 | 1.5993 | 2.21 | 78.8 | 70.1-85.9 |
| 229831_at | CNTN3 | 1.1953E−07 | 1.6145 | 3.93 | 79 | 70.3-86.1 |
| 204940_at | PLN | 4.1416E−07 | 1.5439 | 2.01 | 78 | 69.2-85.2 |
| 227827_at | SORBS2 | 4.2323E−05 | 1.3016 | 2.1 | 74.2 | 65.1-81.9 |
| 238751_at | SORBS2 | 4.2323E−05 | 1.3015 | 2.1 | 74.2 | 65.1-82 |
| 209209_s_at | PLEKHC1 | 0.0007 | 1.0304 | 1.82 | 69.7 | 60.3-78 |
| 206664_at | SI | 0.0054 | 0.9301 | 2.79 | 67.9 | 58.4-76.3 |

TABLE 4

| Gene | | Signif. | D.val | | Sens- | CI |
|---|---|---|---|---|---|---|
| Symbol | ValidPS_DOWN | FDR | 5 | FC | Spec | (95) |
| ANK2 | 314086-HuGene_st: 282874-HuGene_st: 382431- | 2.59E−16 | 2.6515 | 6.76 | 90.8 | 84.1-95.1 |
| | HuGene_st: 229308-HuGene_st: 779600-HuGene_st: 297624- | | | | | |
| | HuGene_st: 385943-HuGene_st: 730140-HuGene_st: 442277- | | | | | |
| | HuGene_st: 699309-HuGene_st: 182816- | | | | | |
| | HuGene_st: 202921_s_at: 799860-HuGene_st: 868462- | | | | | |
| | HuGene_st: 634421-HuGene_st: 571536-HuGene_st: 1050903- | | | | | |
| | HuGene_st: 649509-HuGene_st: 239935-HuGene_st: 202920_at | | | | | |
| DMD | 945600-HuGene_st: 12596-HuGene_st: 434657- | 5.01E−11 | 2.0162 | 4.74 | 84.3 | 76.3-90.4 |
| | HuGene_st: 676962-HuGene_st: 1020969-HuGene_st: 170680- | | | | | |
| | HuGene_st: 855670-HuGene_st: 909848-HuGene_st: 134644- | | | | | |
| | HuGene_st: 887814-HuGene_st: 649184-HuGene_st: 110990- | | | | | |
| | HuGene_st: 914217-HuGene_st: 961622-HuGene_st: 514011- | | | | | |
| | HuGene_st: 842792-HuGene_st: 445483- | | | | | |
| | HuGene_st: 208086_s_at: 987362-HuGene_st: 997308- | | | | | |
| | HuGene_st | | | | | |
| ABCA8 | 57305-HuGene_st: 111752-HuGene_st: 61357- | 1.26E−18 | 2.9182 | 6.82 | 92.8 | 86.8-96.5 |
| | HuGene_st: 123451-HuGene_st: 740746-HuGene_st: 512280- | | | | | |
| | HuGene_st: 389185-HuGene_st: 427721-HuGene_st: 346028- | | | | | |
| | HuGene_st: 224566-HuGene_st: 149653-HuGene_st: 680699- | | | | | |
| | HuGene_st: 76772-HuGene_st: 742091-HuGene_st: 423333- | | | | | |
| | HuGene_st: 559944-HuGene_st: 341399- | | | | | |
| | HuGene_st: 1565780_at: 863400-HuGene_st: 921748- | | | | | |
| | HuGene_st: 623719-HuGene_st: 204719_at: 123143- | | | | | |
| | HuGene_st: 1077391-HuGene_st | | | | | |
| TCF21 | 804657-HuGene_st: 106365-HuGene_st: 710514- | 2.51E−13 | 2.3037 | 2.34 | 87.5 | 80.1-92.9 |
| | HuGene_st: 299556-HuGene_st: 608149-HuGene_st: 242233- | | | | | |
| | HuGene_st: 655881-HuGene_st: 356773-HuGene_st: 788445- | | | | | |
| | HuGene_st: 709897-HuGene_st: 605488-HuGene_st: 652466- | | | | | |
| | HuGene_st: 204931_at: 830709-HuGene_st: 273418- | | | | | |
| | HuGene_st: 1004754-HuGene_st: 990861-HuGene_st: 238739- | | | | | |
| | HuGene_st: 836241-HuGene_st: 110045- | | | | | |
| | HuGene_st: 229529_at: 1001969-HuGene_st | | | | | |
| PLN | 204938_s_at: 418214-HuGene_st: 769837-HuGene_st: 603329- | 4.14E−07 | 1.5399 | 2.01 | 77.9 | 69.2-85.2 |
| | HuGene_st: 1048096-HuGene_st: 944510-HuGene_st: 430402- | | | | | |
| | HuGene_st: 86633-HuGene_st: 795093-HuGene_st: 377757- | | | | | |
| | HuGene_st: 770145-HuGene_st: 920628-HuGene_st: 594048- | | | | | |
| | HuGene_st: 800583-HuGene_st: 204939_s_at: 556723- | | | | | |
| | HuGene_st: 818339-HuGene_st: 175524-HuGene_st: 498924- | | | | | |
| | HuGene_st: 204940_at | | | | | |
| BCHE | 228090-HuGene_st: 752051-HuGene_st: 800167- | 3.18E−13 | 2.2544 | 6.21 | 87 | 79.4-92.5 |
| | HuGene_st: 221362-HuGene_st: 155900-HuGene_st: 717363- | | | | | |
| | HuGene_st: 536584-HuGene_st: 146780-HuGene_st: 302487- | | | | | |
| | HuGene_st: 508472-HuGene_st: 516293-HuGene_st: 968992- | | | | | |
| | HuGene_st: 666625-HuGene_st: 923158-HuGene_st | | | | | |
| P2RY14 | 528057-HuGene_st: 780310-HuGene_st: 235178- | 1.31E−10 | 1.9805 | 2 | 83.9 | 75.9-90 |
| | HuGene_st: 352954-HuGene_st: 699489-HuGene_st: 38001- | | | | | |
| | HuGene_st: 637791-HuGene_st: 25606-HuGene_st: 40647- | | | | | |
| | HuGene_st: 896487-HuGene_st: 672149-HuGene_st: 863820- | | | | | |
| | HuGene_st: 352427-HuGene_st: 632821-HuGene_st: 116148- | | | | | |
| | HuGene_st: 561792-HuGene_st: 840910-HuGene_st: 296420- | | | | | |
| | HuGene_st: 974643-HuGene_st: 206637_at | | | | | |
| PYY | 656845-HuGene_st: 816022-HuGene_st: 633572- | 3.50E−15 | 2.4828 | 11.71 | 89.3 | 82.2-94 |
| | HuGene_st: 20355-HuGene_st: 240779-HuGene_st: 638358- | | | | | |
| | HuGene_st: 879780- | | | | | |
| | HuGene_st: 207080_s_at: 211253_x_at: 368591-HuGene_st | | | | | |
| CITED2 | 125201-HuGene_st: 410723-HuGene_st: 463405- | 5.29E−13 | 2.269 | 1.64 | 87.2 | 79.7-92.6 |
| | HuGene_st: 401168-HuGene_st: 1012057-HuGene_st: 235057- | | | | | |
| | HuGene_st: 361772-HuGene_st: 207980_s_at: 1091907- | | | | | |
| | HuGene_st: 985355-HuGene_st: 175990- | | | | | |
| | HuGene_st: 227287_at: 48433-HuGene_st: 209357_at: 477746- | | | | | |
| | HuGene_st: 243264-HuGene_st: 904401-HuGene_st: 328536- | | | | | |
| | HuGene_st: 1095110-HuGene_st: 89784-HuGene_st: 206734- | | | | | |
| | HuGene_st: 927615-HuGene_st | | | | | |
| GPM6B | 599862-HuGene_st: 754598-HuGene_st: 503642- | 4.04E−12 | 2.1547 | 3.59 | 85.9 | 78.2-91.7 |
| | HuGene_st: 224935-HuGene_st: 754449-HuGene_st: 577489- | | | | | |
| | HuGene_st: 1073242-HuGene_st: 560873-HuGene_st: 1003662- | | | | | |
| | HuGene_st: 430217-HuGene_st: 903323-HuGene_st: 231962- | | | | | |
| | HuGene_st: 244945-HuGene_st: 562460-HuGene_st: 583561- | | | | | |
| | HuGene_st: 209168_at | | | | | |
| PLEKHC1 | 831786-HuGene_st: 88016-HuGene_st: 1087035- | 7.00E−04 | 1.0307 | 1.82 | 69.7 | 60.4-78 |
| | HuGene_st: 530331-HuGene_st: 209209_s_at 184398- | | | | | |
| | HuGene_st: 904923-HuGene_st: 193550-HuGene_st: 468805- | | | | | |
| | HuGene_st: 104495-HuGene_st: 604317-HuGene_st: 501652- | | | | | |
| | HuGene_st: 118096-HuGene_st: 321532-HuGene_st: 675257- | | | | | |
| | HuGene_st: 79496-HuGene_st: 590867-HuGene_st: 147248- | | | | | |
| | HuGene_st: 627402-HuGene_st: 377514-HuGene_st | | | | | |
| ADH1B | 1078343-HuGene_st: 512808: HuGene_st: 614446- | 4.70E−19 | 3.0174 | 4.67 | 93.4 | 87.7-96.9 |
| | HuGene_st: 910188-HuGene_st: 422504-HuGene_st: 731361- | | | | | |
| | HuGene_st: 209612_s_at: 258079-HuGene_st: 568239- | | | | | |
| | HuGene_st: 879930-HuGene_st: 420417-Hu-Gene_st: 1025048- | | | | | |
| | HuGene_st: 908335-HuGene_st: 654633-HuGene_st: 947292- | | | | | |
| | HuGene_st: 1087125-HuGene_st: 1004870- | | | | | |
| | HuGene_st: 209613_s_at: 579636-HuGene_st: 681018- | | | | | |
| | HuGene_st: 822774-HuGene_st | | | | | |
| XLKD1 | 520080-HuGene_st: 1091117-HuGene_st: 943125- | 3.60E−19 | 2.9461 | 7.44 | 93 | 87.1-96.6 |
| | HuGene_st: 444068-HuGene_st: 64855-8-HuGene_st: 346991- | | | | | |
| | HuGene_st: 1006205-HuGene_st: 373107-HuGene_st: 682535- | | | | | |
| | HuGene_st: 1083245-HuGene_st: 863143-HuGene_st: 820120- | | | | | |
| | HuGene_st: 1044561-HuGene_st: 220037_s_at: 541228- | | | | | |
| | HuGene_st: 220256-HuGene_st: 289122- | | | | | |
| | HuGene_st: 219059_s_at: 246683-HuGene_st: 775976- | | | | | |
| | HuGene_st: 207399-HuGene_st: 1052557-HuGene_st: 92121- | | | | | |
| | HuGene_st | | | | | |
| LRRC19 | 177641-HuGene_st: 1070020-HuGene_st: 1055140- | 6.75E−12 | 2.0779 | 4.51 | 85.1 | 77.1-91 |
| | HuGene_st: 525999-HuGene_st: 937256-HuGene_st: 620791- | | | | | |
| | HuGene_st: 891251-HuGene_st: 707559-HuGene_st: 892056- | | | | | |
| | HuGene_st: 764919-HuGene_st: 382143-HuGene_st: 52584- | | | | | |
| | HuGene_st: 920414-HuGene_st: 1028155-HuGene_st: 755055- | | | | | |
| | HuGene_st: 678651-HuGene_st: 1080156-HuGene_st: 530282- | | | | | |
| | HuGene_st: 523877-HuGene_st: 335198-HuGene_st: 787709- | | | | | |
| | HuGene_st: 153175-HuGene_st: 220376_at | | | | | |
| SDPR | 878908-HuGene_st: 781527-HuGene_st: 331976- | 9.62E−08 | 1.6036 | 2.21 | 78.9 | 70-85.9 |
| | HuGene_st: 238150-HuGene_st: 306039-HuGene_st: 535903- | | | | | |
| | HuGene_st: 302361-HuGene_st: 1005813-HuGene_st: 71118- | | | | | |
| | HuGene_st: 992629-HuGene_st: 218711_s_at: 293110- | | | | | |
| | HuGene_st: 779040-HuGene_st: 222717_at: 970479- | | | | | |
| | HuGene_st: 581654-HuGene_st | | | | | |
| TRPM6 | 767074-HuGene_st: 695352-HuGene_st: 411125- | 2.25E−19 | 3.0111 | 7.5 | 93.4 | 87.7-96.8 |
| | HuGene_st: 221102_s_at: 234864_s_at: 240389_at: 358229- | | | | | |
| | HuGene_st: 755964-HuGene_st: 840301-HuGene_st: 959234- | | | | | |
| | HuGene_st: 782639-HuGene_st: 833079-HuGene_st: 1066034- | | | | | |
| | HuGene_st: 678013-HuGene_st: 249083-HuGene_st: 143934- | | | | | |
| | HuGene_st: 159130-HuGene_st: 486486-HuGene_st: 185057- | | | | | |
| | HuGene_st: 878793-HuGene_st: 133981- | | | | | |
| | HuGene_st: 224412_s_at: 202194-HuGene_st | | | | | |
| LIFR | 275506-HuGene_st: 323055-HuGene_st: 444251- | 2.45E−17 | 2.7814 | 6.36 | 91.8 | 85.5-95.8 |
| | HuGene_st: 1056178-HuGene_st: 398104-HuGene_st: 917434- | | | | | |
| | HuGene_st: 1044918-HuGene_st: 167500-HuGene_st: 423760- | | | | | |
| | HuGene_st: 837336-HuGene_st: 321505-HuGene_st: 918321- | | | | | |
| | HuGene_st: 252278-HuGene_st: 884504-HuGene_st: 124845- | | | | | |
| | HuGene_st: 499777-HuGene_st: 969722-HuGene_st: 709439- | | | | | |
| | HuGene_st: 611505-HuGene_st: 227771_at: 287217- | | | | | |
| | HuGene_st: 205876_at: 225571_at: 229185_at: 233367_at | | | | | |
| | 1093011-HuGene_st | | | | | |
| AKAP12 | 212419-HuGene_st: 231067_s_at: 379659- | 7.41E−09 | 1.7456 | 2.83 | 80.9 | 72.4-87.6 |
| | HuGene_st: 1010338-HuGene_st: 1075094-HuGene_st: 42401- | | | | | |
| | HuGene_st: 522584-HuGene_st: 480972-HuGene_st: 948623- | | | | | |
| | HuGene_st: 701945-HuGene_st: 276784-HuGene_st: 64858- | | | | | |
| | HuGene_st: 210517_s_at: 874382-HuGene_st: 909976- | | | | | |
| | HuGene_st: 182037-HuGene_st: 417182-HuGene_st: 722881- | | | | | |
| | HuGene_st | | | | | |
| CLDN23 | 403960-HuGene_st: 25144-HuGene_st: 947653- | 4.27E−20 | 3.0748 | 3.47 | 93.8 | 88.2-97.1 |
| | HuGene_st: 228704_s_at: 228706_s_at: 320375- | | | | | |
| | HuGene_st: 441629-HuGene_st: 367414-HuGene_st: 855269- | | | | | |
| | HuGene_st: 228707_at: 788659-HuGene_st: 698816- | | | | | |
| | HuGene_st: 95789-HuGene_st: 270197-HuGene_st: 472976- | | | | | |
| | HuGene_st: 280539-HuGene_st: 1056334-HuGene_st: 516288- | | | | | |
| | HuGene_st: 579963-HuGene_st | | | | | |
| CD36 | 392196-HuGene_st: 274514-HuGene_st: 477005- | 6.27E−14 | 2.4696 | 1.65 | 89.2 | 82.1-94 |
| | HuGene_st: 6915-85-HuGene_st: 872909-HuGene_st: 543050- | | | | | |
| | HuGene_st: 603343-HuGene_st: 514557-HuGene_st: 296850- | | | | | |
| | HuGene_st: 945913-HuGene_st: 495755- | | | | | |
| | HuGene_st: 205488s_at: 1035854-HuGene_st: 887301- | | | | | |
| | HuGene_st: 836370-HuGene_st: 209555_s_at: 939919- | | | | | |
| | HuGene_st: 507440-HuGene_s 151788-HuGene_st: 146280- | | | | | |
| | HuGene_st: 360545-HuGene_st: 1051486- | | | | | |
| | HuGene_st: 228766_at: 512885-HuGene_st | | | | | |
| RPL24 | 1559655_at: 1559656_a_at: 228885_at | 1.23E−14 | 2.5568 | 1.63 | 89.9 | 83.1-94.6 |
| GCNT2 | 935239-HuGene_st: 225205-HuGene_st: 1026280- | 2.16E−27 | 3.9536 | 13.36 | 97.6 | 94.1-99.2 |
| | HuGene_st: 668101-HuGene_st: 1099985-HuGene_st: 698568- | | | | | |
| | HuGene_st: 134540-HuGene_st: 697147-HuGene_st: 250092- | | | | | |
| | HuGene_st: 611927-HuGene_st: 972833-HuGene_st: 168891- | | | | | |
| | HuGene_st: 990860-HuGene_st: 109287-HuGene_st: 322116- | | | | | |
| | HuGene_st: 231019-HuGene_st: 211020_at: 959570- | | | | | |
| | HuGene_st: 858764-HuGene_st: 215593_at 820195- | | | | | |
| | HuGene_st: 239606_at: 41059-HuGene_st: 669940- | | | | | |
| | HuGene_st: 215595_x_at: 230788_at | | | | | |
| PKIB | 866170-HuGene_st: 1055812-HuGene_st: 264946- | 5.94E−20 | 3.1204 | 3.28 | 94.1 | 88.6-97.3 |
| | HuGene_st: 684057-HuGene_st: 124791-HuGene_st: 134561- | | | | | |
| | HuGene_st: 1026756-HuGene_st: 468593-HuGene_st: 1045852- | | | | | |
| | HuGene_st: 939917-HuGene_st: 110205-HuGene_st: 660721- | | | | | |
| | HuGene_st: 905229-HuGene_st: 223551_at: 610426-HuGene_st | | | | | |
| ANGPTL1 | 572942-HuGene_st: 891312-HuGene_st: 953040- | 1.42E−17 | 2.7964 | 4.64 | 91.9 | 85.6-95.9 |
| | HuGene_st: 232844-HuGene_st: 145730-HuGene_st: 142205- | | | | | |
| | HuGene_st: 227771-HuGene_st: 80584-HuGene_st: 982090- | | | | | |
| | HuGene_st: 999640-HuGene_st: 672931-HuGene_st: 148578- | | | | | |
| | HuGene_st: 224339_s_at: 1046706- | | | | | |
| | HuGene_st: 239183_at: 155600-HuGene_st: 284674- | | | | | |
| | HuGene_st: 231773_at: 818064-HuGene_st: 978991- | | | | | |
| | HuGene_st: 728775-HuGene_st | | | | | |
| SI | 9605-HuGene_st: 514814-HuGene_st: 809314- | 5.40E−03 | 0.9299 | 2.79 | 67.9 | 58.4-76.4 |
| | HuGene_st: 576613-HuGene_st: 45-502-HuGene_st: 369676- | | | | | |
| | HuGene_st: 438636-HuGene_st: 267204-HuGene_st: 326716- | | | | | |
| | HuGene_st: 897825-HuGene_st: 377666-HuGene_st: 519273- | | | | | |
| | HuGene_st: 325741-HuGene_st: 245381-HuGene_st: 108368- | | | | | |
| | HuGene_st: 464198-HuGene_st: 679193-HuGene_st: 168180- | | | | | |
| | HuGene_st | | | | | |
| HPGD | 291863-HuGene_st: 375608-HuGene_st: 793406- | 3.65E−17 | 2.8462 | 3.21 | 92.3 | 86.2-96.1 |
| | HuGene_st: 436293-HuGene_st: 75568- | | | | | |
| | HuGene_st: 211549_s_at: 684728-HuGene_st: 674596- | | | | | |
| | HuGene_st: 527856-HuGene_st: 329920-HuGene_st: 748432- | | | | | |
| | HuGene_st: 259392-HuGene_st: 769902-HuGene_st: 620673- | | | | | |
| | HuGene_st: 450707-HuGene_st: 203913_s_at: 304752- | | | | | |
| | HuGene_st: 447604-HuGene_st: 170968-HuGene_st: 852359- | | | | | |
| | HuGene_st: 836377-HuGene_st: 242733_at: 243846- | | | | | |
| | HuGene_st: 136281- | | | | | |
| | HuGene_st: 203914_x_at: 211548_s_at: 288252-HuGene_st | | | | | |
| CLDN8 | 1018006-HuGene_st: 190634-Hu -dene_st: 590280- | 2.92E−10 | 1.8639 | 12.96 | 82.4 | 74.1-88.9 |
| | HuGene_st: 186468-HuGene_st: 954438-HuGene_st: 428391- | | | | | |
| | HuGene_st: 480543-HuGene_st: 944337-HuGene_st: 179725- | | | | | |
| | HuGene_st: 508584-HuGene_st: 1009114-HuGene_st: 948216- | | | | | |
| | HuGene_st: 658285-HuGene_st: 1022600-HuGene_st: 737498- | | | | | |
| | HuGene_st: 470015-HuGene_st: 103315-HuGene_st: 699348- | | | | | |
| | HuGene_st: 89877-HuGene_st: 56937-HuGene_st: 862663- | | | | | |
| | HuGene_st: 504945-HuGene_st: 214598_at | | | | | |
| UGT2A3 | 149647-HuGene_st: 860083-HuGene_st: 922544- | 8.64E−09 | 1.7422 | 4.89 | 80.8 | 72.3-87.5 |
| | HuGene_st: 244206-HuGene_st: 503323-HuGene_st: 353576- | | | | | |
| | HuGene_st: 603619-HuGene_st: 787458-HuGene_st: 219796- | | | | | |
| | HuGene_st: 333564-HuGene_st: 257402-HuGene_st: 366699- | | | | | |
| | HuGene_st: 461685-HuGene_st: 891681-HuGene_st: 644952- | | | | | |
| | HuGene_st: 621618-HuGene_st: 737617-HuGene_st: 88682- | | | | | |
| | HuGene_st: 529761-HuGene_st: 895203-HuGene_st: 658594- | | | | | |
| | HuGene_st: 455115-HuGene_st | | | | | |
| HHLA2 | 371335-HuGene_st: 978721-HuGene_st: 1065567- | 3.47E−17 | 2.6743 | 10.02 | 90.9 | 84.3-95.2 |
| | HuGene_st: 2825-48-HuGene_st: 240410-HuGene_st: 170899- | | | | | |
| | HuGene_st: 947848-HuGene_st: 438234- | | | | | |
| | HuGene_st: 220812_s_at: 927495-HuGene_st: 351364- | | | | | |
| | HuGene_st: 234673_at: 993142-HuGene_st: 1009637- | | | | | |
| | HuGene_st: 335000-HuGene_st: 285313: HuGene_st: 533646- | | | | | |
| | HuGene_st: 234624_at: 458597-HuGene_st: 104838- | | | | | |
| | HuGene_st: 26687-HuGene_st: 258409-HuGene_st: 493304- | | | | | |
| | HuGene_st: 378019-HuGene_st: 576796-HuGene_st | | | | | |
| UGT1A8 | 116025-HuGene_st: 6488-Hu-Gene_st: 881135- | 1.32E−11 | 2.0703 | 4.56 | 85 | 77.1-90.9 |
| | HuGene_st: 230953_at: 221304_at: 221305_s_at: 511516- | | | | | |
| | HuGene_st: 5949637-HuGene_st: 396121-HuGene_st: 123777- | | | | | |
| | HuGene_st: 1016481-HuGene_st: 204532_x_at: 683377- | | | | | |
| | HuGene_st: 1055169-HuGene_st: 1035103- | | | | | |
| | HuGene_st: 1088102-HuGene_st: 207126_x_at: 42874- | | | | | |
| | HuGene_st: 215125_s_at: 206094_x_at: 208596_s_at: 97211- | | | | | |
| | HuGene_st: 1009861-HuGene_st: 603368- | | | | | |
| | HuGene_st: 232654_s_at: 6258-97-HuGene_st: 998604- | | | | | |
| | HuGene_st | | | | | |
| CNTN3 | 267567-HuGene_st: 782053-HuGene_st: 550157- | 1.20E−07 | 1.6155 | 3.93 | 79 | 70.3-86.1 |
| | HuGene_st: 541394-HuGene_st: 989173-HuGene_st: 15899- | | | | | |
| | HuGene_st: 78622-HuGene_st: 339738-HuGene_st: 585282- | | | | | |
| | HuGene_st: 661814-HuGene_st: 360715-HuGene_st: 695033- | | | | | |
| | HuGene_st: 483058-HuGene_st: 555394-HuGene_st: 315011- | | | | | |
| | HuGene_st: 905374-HuGene_st: 1067212-HuGene_st: 557263- | | | | | |
| | HuGene_st: 233502_at: 811729-HuGene_st: 87414-HuGene_st | | | | | |
| P2RY1 | 628249-HuGene_st: 113454-HuGene_st: 627857- | 1.41E−15 | 2.5326 | 3.6 | 89.7 | 82.8-94.4 |
| | HuGene_st: 461281-HuGene_st: 207455_at: 259065- | | | | | |
| | HuGene_st: 797734-HuGene_st: 135788-HuGene_st: 42916- | | | | | |
| | HuGene_st: 315405-HuGene_st: 340050-HuGene_st: 173225- | | | | | |
| | HuGene_st: 919818-HuGene_st: 591228-HuGene_st: 899117- | | | | | |
| | HuGene_st: 785070-HuGene_st: 286200-HuGene_st: 231925_at | | | | | |
| SORBS2 | 238751_at: 805920-HuGene_st | 4.23E−05 | 1.3026 | 2.1 | 74.3 | 65.1-82 |

TABLE 5

| | Normal | | | | | |
|---|---|---|---|---|---|---|
| Gene | Blood | MRC5 | CaCo2 | HCT116 | HT29 | SW480 |
| CXCL12 | Unmeth | low | unmeth | meth | meth | unmeth |
| DF | Low | meth | meth | meth | unmeth | meth |
| MAMDC2 | Unmeth | unmeth | meth | unmeth | unmeth | meth |
| CA4 | unmeth | unmeth | meth | meth | meth | unmeth |
| MT1M | low | low | low | meth | low | meth |
| P2RY14 | unmeth | Low | Meth | Meth | Unmeth | Meth |
| GPM6B | Low | Low | Low | Meth | Meth | Meth |
| ADAMDEC1 | low | Unmeth | Meth | Meth | unmeth | meth |

TABLE 6

| | Probe | |
|---|---|---|
| | Set ID | Target Sequence |
| 1 | 200600 | tagtcttggggcaggatgattttggcctcattactttaccacccccacacctggaaagcatatactatattacaaaatgacattttgcc |
| | _at | aaaattattaatataagaagctttcagtattagtgatgtcatctgtcactataggtcatacaatccattcttaaagtacttgttatttg |
| | | tttttattattactgtttgtcttctccccagggttcagtccctcaaggggccatcctgtcccaccatgcagtgccccctagcttagagc |
| | | ctccctcaattccccctggccaccaccccccactctgtgcctgaccttgaggagtcttgtgtgcattgctgtgaattagctcacttggt |
| | | gatatgtcctatattggctaaattgaaacctggaattgtggggcaatctattaatagctgccttaaagtcagtaacttacccttaggga |
| | | ggctgggggaaaaggttagattttgtattcaggggttttttgtgtactttttgggtttttaaaaaattgtttttggaggggtttatgct |
| | | caatccatgt |
| 2 | 200621 | gatagcctcccactaggactgggaggagaataacccaggtcttaaggaccccaaagtcaggatgttgtttgatcttctcaaacatctag |
| | _at | ttccctgcttgatgggaggatcctaatgaaatacctgaaacatatattggcatttatcaatggctcaaatcttcatttatctctggcct |
| | | taaccctggctcctgaggctgcggccagcagagcccaggccagggctctgttcttgccacacctgcttgatcctcagatgtggagggag |
| | | gtaggcactgcctcagtcttcatccaaacacctttccctttgccctgagacctcagaatcttccctttaacccaagaccctgcctcttc |
| | | cactccacccttctccagggacccttagatcacatcactccacccctgccaggccccaggttaggaatagtggtgggaggaaggggaaa |
| | | gggctgggcctcaccgctcccagcaactgaaaggacaacactatctggagccacccactgaaagggctgcaggcatgggctgtac |
| 3 | 200665 | gttggttcaaacttttgggagcacggactgtcagttctctgggaagtggtcagcgcatcctgcagggcttctcctcctctgtcttttgg |
| | _s_at | agaaccagggctcttctcaggggctctagggactgccaggctgtttcagccaggaaggccaaaatcaagagtgagatgtagaaagttgt |
| | | aaaatagaaaaagtggagttggtgaatcggttgttctttcctcacatttggatgattgtcataaggtttttagcatgttcctccttttc |
| | | ttcaccctcccctttgttcttctattaatcaagagaaacttcaaagttaatgggatggtcggatctcacaggctgagaactcgttcacc |
| | | tccaagcatttcatgaaaaagctgcttcttattaatcatacaaactctcaccatgatgtg |
| 4 | 200795 | aacactgcataacccgtttctttgaggagtgtgaccccaacaaggataagcacatcaccctgaaggagtggggccactgctttggaatt |
| | _at | aaagaagaggacatagatgaaaatctcttgttttgaacgaagattttaaagaactcaactttccagcatcctcctctgttctaaccact |
| | | tcagaaatatatgcagctgtgatacttgtagatttatatttagcaaaatgttagcatgtatgacaagacaatgagagtaattgcttgac |
| | | aacaacctatgcaccaggtatttaacattaactttggaaacaaaaatgtacaattaagtaaagtcaacatatgcaaaatactgtacatt |
| | | gtgaacagaagtttaattcatagtaatttcactctctgcattgacttatgagataattaatgattaaactattaatgataaaaataatg |
| | | catttgtattgttcataatatcatgtgcacttcaagaaaatggaatgctactcttttgtggtttac |
| 5 | 200799 | ggccgacaagaagaaggtgctggacaagtgtcaagaggtcatctcgtggctggacgccaacaccttggccgagaaggacgagtttgagc |
| | _at | acaagaggaaggagctggagcaggtgtgtaaccccatcatcagcggactgtaccagggtgccggtggtcccgggcctgggggcttcggg |
| | | gctcagggtcccaagggagggtctgggtcaggccccaccattgaggaggtagattaggggcctttccaagattgctgtttttgttttgg |
| | | agcttcaagactttgcatttcctagtatttctgtttgtcagttctcaatttcctgtgtttgcaatgttgaaattttttggtgaagtact |
| | | gaacttgctttttttccggtttctacatgcagagatgaatttatactgccatcttacgactatttcttctttttaatacacttaactca |
| | | ggccatttttt |
| 6 | 200845 | tcctgtcacccattttgaagagtggcagaacttgaagttcaacttcctctgtaaatatccaagtataaagcccaggaacttctagaata |
| | _s_at | acccagatgcgctttaattttttttaatatgttttgatcacagaacttctagaataacccagatgctctttcatattcttttaatacat |
| | | cttgatcacagctgggggaaaaaaagctttttaattctgtaccttcctagtagataagtgaagagcagggaaagagacctttaaatatt |
| | | ttgctataaaaaaatttgtgataagtttctatcaaaatggggagattgcagaaaaggcttcccttggctcccaaggaggtgtagcaggt |
| | | gtgagcaatattagtgccatgtgcctttcacacagggtttgcatttatcagtctgttttccgatgatgtgtacatgaaagagtacacca |
| | | tgtgaagagaagagagaatgattgaaaatgttttagtatagaactcttcttgcagtgggttgctattttctagatttta |
| 7 | 200859 | gtttgtagactctctgaccaaggccacctgtgccccccagcatggggccccgggtcctgggcctgctgacgccagcaaggtggtggcca |
| | _x_at | agggcctggggctgagcaaggcctacgtaggccagaagagcagcttcacagtagactgcagcaaagcaggcaacaacatgctgctggtg |
| | | ggggttcatggcccaaggaccccctgcgaggagatcctggtgaagcacgtgggcagccggctctacagcgtgtcctacctgctcaagga |
| | | caagggggagtacacactggtggtcaaatgggggcacgagcacatcccaggcagcccctaccgcgttgtggtgccctgagtctggggcc |
| | | cgtgccagccggcagcccccaagcctgccccgctacccaagcagccccgccctcttcccctcaaccccggcccaggccgccctggccgc |
| | | ccgcctgtcactgcagctgcccctgccctgtgccgtgctgcgctcacctgcctccccagccagccgctgacctctcggctttcacttgg |
| | | gcagagggagccatttggtggcgctgct |
| 8 | 200884 | cctcacccagattgaaactctcttcaagtctaaggactatgagttcatgtggaaccctcacctgggctacatcctcacctgcccatcca |
| | _at | acctgggcaccgggctgcgggcaggtgtcgatatcaagctgcccaacctgggcaagcatgagaagttctcggaggtgcttaagcggctg |
| | | cgacttcagaagcgaggcacaggcggtgtggacacggctgcggtgggcggggtcttcgacgtctccaacgctgaccgcctgggcttctc |
| | | agaggtggagctggtgcagatggtggtggacggagtgaagctgctcatcgagatggaacagcggctggagcagggccaggccatcgacg |
| | | acctcatgcctgcccagaaatgaagcccggcccacacccgacaccagccctgctgcttcctaacttattgcctgcagtgcccaccatgc |
| | | acccctcgatgttgccgtctggcgagcccttagccttgctgtaaggaaggcttccgtcacccttggtagagtttat |
| 9 | 200897 | ctctcttagctcagttactcaattcatacgtagtattttttaaaataattttatatctgtgtaccaccccatatatttcatattactgt |
| | _s_at | ttcacatgtacagctttctacttctttgtaagaacaccaaccaaccaaggtttaagtgattaataggcttgagcaccgggtggcagatg |
| | | ttctatgcagtgtggttcaagtttctttgaccgcacttatatgcattgctaatatggaatttaagataccatacacagtctctcatgga |
| | | cctatctctattgtagaattatgacttatgtcttacttggcaaatttttctgaatgtgacctttttttgctgatttgctgggtttggga |
| | | ttaactagcattattttgccacctt |
| 10 | 200974 | aagagttacgagttgcctgatgggcaagtgatcaccatcggaaatgaacgtttccgctgcccagagaccctgttccagccatccttcat |
| | _at | cgggatggagtctgctggcatccatgaaaccacctacaacagcatcatgaagtgtgatattgacatcaggaaggacctctatgctaaca |
| | | atgtcctatcagggggcaccactatgtaccctggcattccgaccgaatgcagaaggagatcacggccctagcacccagcaccatgaaga |
| | | tcaagatcattgcccctccggagcgcaaatactctgtctggatcggtggctccatcctggcctctctgtccaccttccagcagatgtgg |
| | | atcagcaaacaggaatacgatgaagccgggccttccattgtccaccgcaaatgcttctaaaacactttcctgctcctctctgtctctag |
| | | cacacaactgtgaatgtcctgtggaattatgccttcagttcttttccaaatcattcctagccaaagctctgactcgt |
| 11 | 200986 | gacgaccagccaggatatgctctcaatcatggagaaattggaattcttcgatttttcttatgaccttaacctgtgtgggctgacagagg |
| | _at | acccagatcttcaggtttctgcgatgcagcaccagacagtgctggaactgacagagactggggtggaggcggctgcagcctccgccatc |
| | | tctgtggcccgcaccctgctggtctttgaagtgcagcagcccttcctcttcgtgctctgggaccagcagcacaagttccctgtcttcat |
| | | ggggcgagtatatgaccccagggcctgagacctgcaggatcaggttagggcgagcgctacctctccagcctcagctctcagttgcagcc |
| | | ctgctgctgcctgcctggacttgcccctgccacctcctgcctcaggtgtccgctatccaccaaaagggctcctgagggtctgggcaagg |
| | | gacctgcttctattagcccttctccatggccctgccatgctctccaaaccactttttgcagctttctc |
| 12 | 201041 | gcaataactctgggaggggctcgagagggctggtccttatttatttaacttcacccgagttcctctgggtttctaagcagttatggtga |
| | _s_at | tgacttagcgtcaagacatttgctgaactcagcacattcgggaccaatatatagtgggtacatcaagtccatctgacaaaatggggcag |
| | | aagagaaaggactcagtgtgtgatccggtttctttttgctcgcccctgttttttgtagaatctcttcatgcttgacatacctaccagta |
| | | ttattcccgacgacacatatacatatgagaatataccttatttatttttgtgtaggtgtctgccttcacaaatgtcattgtctactcct |
| | | agaa |
| 13 | 201058 | gcatcctcaaacatggcgccaaggataaacacgactaggccatccccagccccctgacacccagcccccgccagtcacccctccccgca |
| | _s_at | cacacccgtccataccagctccctgcccatgaccctcgctcagggatccccctttgagggttagggtcccagttcccagtggaagaaac |
| | | aggccaggagagtgcgtgccgagctgaggcagatgttcccacagtgaccccagagccctgggctatagtctctgacccctccaaggaaa |
| | | gaccaccttctggggacatgggctggagggcaggacctagaggcaccaagggaaccgcattccggggctgttccccgaggaggaaggga |
| | | agcctctgtgtgccccccaggaggaagaggccctgagtcctgggatcagacaccccttcacgtgtatcccacacaaatgcaagctcacc |
| | | aaggtccc |
| 14 | 201061 | tatagctctctttagctcaaccactctgtccatccagccaatggatgtccttccctgtacccaattcaagcttattttagggaagcctt |
| | s_at | gaaactaccatgtatctggctctagctgagttattgaggattgagccagtgcaacgttaaactcagtgcacttacatttgatttaaatg |
| | | atggttttatctgttgtgtgaagtggttcacccttgaggaccaggagcctccatatcctgactgaaaaccttttctgagacttagagta |
| | | acagtacttttggttccttgagttctcctgtctccagatacctaaatgaccttgacttttctgccttgtgaattcgtagtccaatcagc |
| | | tgaaattaaatcacttgggagggacgcatagaaggagctctaggaacacagtgccagtgcagaagtttctccaggtggcc |
| 15 | 201069 | ctcagagccacccctaaagagatcctttgatattttcaacgcagccctgctttgggctgccctggtgctgccacacttcaggctcttct |
| | _at | cctttcacaaccttctgtggctcacagaacccttggagccaatggagactgtctcaagagggcactggtggcccgacagcctggcacag |
| | | ggcagtgggacagggcatggccaggtggccactccagacccctggcttttcactgaggctgccttagaacctttcttacattagcagtt |
| | | tgctttgtatgcactttgtttttttctttgggtcttgttttttttttccacttagaaattgcatttcctgacagaaggactcaggttgt |
| | | ctgaagtcactgcacagtgcatctcagcccacatagtgatggttcccctgttcactctacttagcatgtccctaccgagtctcttctcc |
| | | actggatggaggaaaaccaagccgtggcttcccgctcagccctccctgcccctcccttcaaccattccccatgggaaat |
| 16 | 201105 | ctcctggactcaatcatggcttgtggtctggtcgccagcaacctgaatctcaaacctggagagtgccttcgagtgcgaggcgaggtggc |
| | _at | tcctgacgctaagagcttcgtgctgaacctgggcaaagacagcaacaacctgtgcctgcacttcaaccctcgcttcaacgcccacggcg |
| | | acgccaacaccatcgtgtgcaacagcaaggacggcggggcctgggggaccgagcagcgggaggctgtattcccttccagcctggaagtg |
| | | ttgcagaggtgtgcatcaccttcgaccaggccaacctgaccgtcaagctgccagatggatacgaattcaagttccccaaccgcctcaac |
| | | ctggaggccatcaactacatggcagctgacggtgacttcaa |
| 17 | 201137 | ggcagcattcaagtccgatggttcctgaatggacaggaggaaacagctggggtcgtgtccaccaacctgatccgtaatggagactggac |
| | _s_a | catccagatcctggtgatgctggaaatgaccccccagcagggagacgtctacatctgccaagtggagcacaccagcctggatagttccg |
| | | tcaccgtggagtggaaggcacagtctgattctgcccagagtaagacattgacgggagctgggggcttcgtgctggggctcatcatctgt |
| | | ggagtgggactcttcatgcacaggaggagcaagaaagttcaacgaggatctgcataaacagggttcctgacctcaccgaaaagactaat |
| | | gtgccttagaacaagcatttgctgtgttttgttaacacctggttccaggacagaccctcagcttcccaagaggatactgctgccaagaa |
| | | gttgctctgaagtcagtttctatcgttctgctctttgattcaaa |
| 18 | 201141 | agaggcgggatactttcagctttccatgtaactgtatgcataaagccaatgtagtccagtttctaagatcatgttccaagctaactgaa |
| | _at | tcccacttcaatacacactcatgaactcctgatggaacaataacaggcccaagcctgtggtatgatgtgcacacttgctagactcagaa |
| | | aaaatactactctcataaatgggtgggagtattttggtgacaacctactttgcttggctgagtgaaggaatgatattcatatattcatt |
| | | tattccatggacatttagttagtgctttttatataccaggcatgatgctgagtgacactcttgtgtatatttccaaatttttgtatagt |
| | | cgctgcacatatttgaaatcatatattaagactttccaaagatgaggtccctggtttttcatggcaacttgatcagtaaggatttcacc |
| | | tctgtttgtaacta |
| 19 | 201150 | gactttttggaatagccctgtctagggcaaactgtggcccccaggagacactacccttccatgccccagacctctgtcttgcatgtgac |
| | _s_at | aattgacaatctggactaccccaagatggcacccaagtgtttggcttctggctacctaaggttaacatgtcactagagtatttttatga |
| | | gagacaaacattataaaaatctgatggcaaaagcaaaacaaaatggaaagtaggggaggtggatgtgacaacaacttccaaattggctc |
| | | tttggaggcgagaggaaggggagaacttggagaatagtttttgctttgggggtagaggcttcttagattctcccagcatccgcctttcc |
| | | ctttagccagtctgctgtcctgaaacccagaagtgatggagagaaaccaacaagagatctcgaaccctgtctagaaggaatgtatttgt |
| | | tgctaaatttcgtagcactgtttacagttttcctccatgttatttatg |
| 20 | 201289 | gccacgattggagaatactttgcttcatagtattggagcacatgttactgcttcattttggagcttgtggagttgatgactttctgttt |
| | _at | tctgtttgtaaattatttgctaagcatattttctctaggcttttttccttttggggttctacagtcgtaaaagagataataagattagt |
| | | tggacagtttaaagcttttattcgtcctttgacaaaagtaaatgggagggcattccatcccttcctgaagggggacactccatgagtgt |
| | | ctgtgagaggcagctatctgcactctaaactgc |
| 21 | 201300 | ttaggtcaagttcatagtttctgtaattggcttttgaatcaaagaatagggagacaatctaaaaaatatcttaggttggagatgacaga |
| | _s_at | aatatgattgatttgaagtggaaaaagaaattctgttaatgttaattaaagtaaaattattccctgaattgtttgatattgtcacctag |
| | | cagatatgtattacttttctgcaatgttattattggcttgcactttgtgagtatctatgtaaaaatatatatgtatataaaatatatat |
| | | tgcataggacagacttaggagttttgtttagagcagttaacatctgaagtgtctaatgcattaacttttgtaaggtactgaatacttaa |
| | | tatgtgggaaacccttttgcgtggtccttaggcttacaatgtgcactgaatcgtttcatgtaagaatccaaagtggacaccattaacag |
| | | gtctttgaaatatgcatgtactttatattttctatatttgtaactttgcatgttcttgttttgtta |
| 22 | 201324 | caccaaattacctaggctgaggttagagagattggccagcaaaaactgtgggaagatgaactttgtcattatgatttcattatcacatg |
| | _at | attatagaaggctgtcttagtgcaaaaaacatacttacatttcagacatatccaaagggaatactcacattttgttaagaagttgaact |
| | | atgactggagtaaaccatgtattcccttatcttttactttttttctgtgacatttatgtctcatgtaatttgcattactctggtggatt |
| | | gttctagtactgtattgggcttcttcgttaat |
| 23 | 201348 | accagctctaggtccaattgttctgctctaactgatacctcaaccttggggccagcatctcccactgcctccaaatattagtaactatg |
| | _at | actgacgtccccagaagtttctgggtctaccacactccccaaccccccactcctacttcctgaagggccctcccaaggctacatcccca |
| | | ccccacagttctccctgagagagatcaacctccctgagatcaaccaaggcagatgtgacagcaagggccacggaccccatggcaggggt |
| | | ggcgtcttcatga |
| 24 | 201426 | tgtggatgtttccaagcctgacctcacggctgccctgcgtgacgtacgtcagcaatatgaaagtgtggctgccaagaacctgcaggagg |
| | _s_at | cagaagaatggtacaaatccnagtttgctgacctctctgaggctgccaaccggnacnatgacgccctgcgccaggcnaagcaggagtcc |
| | | nctgagtaccggagacaggtgcagtccctcacctgtgaagtggatgcccttaaaggaaccaatgagtccctggaacgccagatgcgtga |
| | | aatggaagagaactttgccgttgaagctgctaactaccaagacactattggccgcctgcaggatgagattcagaatatgaaggaggaaa |
| | | tggctcgtcaccttcgtgaataccaagacctgctcaatgttaagatggcccttgacattgagattgccacct |
| 25 | 201427 | gatccagaaatacttaacacgtgaatattttgctaaaaaagcatatataactattttaaatatccatttatcttttgtatatctaagac |
| | _s_at | tcatcctgatttttactatcacacatgaataaaggcctttgtatctttctttctctaatgttgtatcatactcttctaaaacttgagtg |
| | | gctgtcttaaaagatataaggggaaagataatattgtctgtctctatattgcttagtaagtatttccatagtcaatgatggtttaatag |
| | | gtaaaccaaaccctataaacctgacctcctttatggttaatactattaagcaagaatgcagtacagaattggatacagtacggatttgt |
| | | ccaaat |
| 26 | 201438 | ctccagcagttctctcgaatactttgaatgttgtgtaacagttagccactgctggtgtttatgtgaacattcctatcaatccaaattcc |
| | _at | ctctggagtttcatgttatgcctgttgcaggcaaatgtaaagtctagaaaataatgcaaatgtcacggctactctatatacttttgctt |
| | | ggttcattttttttcccttttagttaagcatgactttagatgggaagcctgtgtatcgtggagaaacaagagaccaactttttcattcc |
| | | ctgcccccaatttcccagactagatttcaagctaattttctttttctgaagcctctaacaaatgatctagttcagaaggaagcaaaatc |
| | | ccttaatctatgtgcaccgttgggaccaatgccttaattaaagaatttaaaaaagttgtaatagagaatatttttggcattcctctcaa |
| | | tgttgtgtgtt |
| 27 | 201495 | caagtgcagcgatggggagcgggcccgggcggagctcaatgacaaagtccacaagctgcagaatgaagttgagagcgtcacagggatgc |
| | _x_at | ttaacgaggccgaggggaaggccattaagctggccaaggacgtggcgtccctcagttcccagctccaggacacccaggagctgcttcaa |
| | | gaagaaacccggcagaagctcaacgtgtctacgaagctgcgccagctggaggaggagcggaacagcctgcaagaccagctggacgagga |
| | | gatggaggccaagcagaacctggagcgccacatctccactctcaacatccagctctccgactcgaagaagaagctgcaggactttgcca |
| | | gcaccgtggaagctctggaagaggggaagaagaggttccagaaggagatcgagaacctcacccag |
| 28 | 201496 | tcaagtccaagttcaagtccaccatcgcggcgctggaggccaagattgcacagctggaggagcaggtcgagcaggaggccagagagaaa |
| | _x_at | caggcggccaccaagtcgctgaagcagaaagacaagaagctgaaggaaatcttgctgcaggtggaggacgagcgcaagatggccgagca |
| | | gtacaaggagcaggcagagaaaggcaatgccagggtcaagcagctcaagaggcagctggaggaggcagaggaggagtcccagcgcatca |
| | | acgccaaccgcaggaagctgcagcgggagnnggatgaggccacggagagcaacgaggccatgggccgcgaggtgaacgcactcaagagc |
| | | aagctcagagggccccccccacaggaaacttcgcagtgatgcaccaggcgaggaaacgagacctctttcgttccttctagaaggtctgg |
| | | aggacgtagagttattgaaaatgcagatggttctgaggaggaaacggacactcgagacgcagacttcaatggaaccaaggccagtgaat |
| | | aagcaactttctacagttttgcaccacgg |
| 29 | 201497 | tgagccaggaagaacgctccagcccaggacttcgaggctgcaatgagctataattgcatcattgcactccagcctgggcaacagagacc |
| | _x_at | ctgtctcaaccaccaccaccaccaccacccctactacccctgtattcaaggtaaaaattgaagtttgtatgatgtaagagatgagaaaa |
| | | acccaacaggaaacacagacacatcctccagttctatcaatggattgtgcagacactgagtttttagaaaaacatatccacggtaaccg |
| | | gtccctggcaattctgtttacatgaaatggggagaaagtcaccgaaatgggtgccgccggcccccactcccaattcattccctaacctg |
| | | caaacctttccaacttctcacgtcaggcctttgagaattctttccccctctcctggtttccacacctcagacacgcacagttcaccaag |
| | | tgccttctgtagtcacatgaattgaaaaggagacgctgctcccacggaggggagcaggaatgctgcactgtttacaccctgactgtgct |
| | | taaaa |
| 30 | 201539 | tgaagtgcaacaaggccatcacatctggaggaatcacttaccaggatcagccctggcatgccgattgctttgtgtgtgttacctgctct |
| | _s_at | aagaagctggctgggcagcgtttcaccgctgtggaggaccagtattactgcgtggattgctacaagaactttgtggccaagaagtgtgc |
| | | tggatgcaagaaccccatcactgg |
| 31 | 201540 | cagggctgtcatcaacatggatatgacatttcacaacagtgactagttgaatcccttgtaacgtagtagttgtctgctctttgtccatg |
| | _at | tgttaatgaggactgcaaagtcccttctgttgtgattcccaggacttttcctcaagaggaaatctggatttccacctaccgcttacctg |
| | | aaatgcaggatcacctacttactgtattctacattattatatgacatagtataatgagacaatatcaaaagtaaacatgtaatgacaat |
| | | acatactaacattcttgtaggagtggttagagaagctgatgcctcatttctacattctgtcattagctattatcatctaacgtttcagt |
| | | g |
| 32 | 201616 | cttgagaccaggagacgtatccagcaagcggaacctctgggaaaagcaatctgtggataaggtcacttcccccactaaggtttgagaca |
| | _s_a | gttccagaaagaacccaagctcaagacgcaggacgagctcagttgtagagggctaattcgctctgttttgtatttatgttgatttacta |
| | | aattgggttcattatcttttatttttcaatatcccagtaaacccatgtatattatcactatatttaataatcacagtctagagatgttc |
| | | atggtaaaagtactgcctttgcacaggagcctgtttctaaagaaacccatgctgtgaaatagagacttttctactgatcatcataactc |
| | | tgtatctgagcagtgataccaaccacatctgaagtcaacagaagatccaagtttaaaattgcctgcggaatgtgtgcagtatctagaaa |
| 33 | 201617 | tgttgtttctgcactttataataaagcatggaagaaattatcttagtaggcaattgtaacactttttgaaagtaacccatttcagattt |
| | _x_at | gaaatactgcaataatggttgtctttaaaaaaaaaaagaatgtactgttaaggtattactttttttcatgctgatgattcatatctaaa |
| | | ttacattattatgttagctgacagtggtactgattttttaggttggttgttttgtggatttctttagtagtgatagtagcctgaaccac |
| | | attttagataactcaattatgtatgtatgtgcatacacatatacaaacacactaatggtagaatgcttttttatgtgctagactattat |
| | | atttagtagtatgtcattgtaactagccaatatcacagcttttgaaaaattaaaaaatcatcatcactataatatttcatatttgccaa |
| | | cagaaacatggcagataggtatcaatatgttttcaatgcctgatgacctat |
| 34 | 201645 | ttttaccaaagcatcaatacaaccagcccaaccatcggtccacacctgggcatttggtgagaatcaaagctgaccatggatccctgggg |
| | _at | ccaacggcaacagcatgggcctcacctcctctgtgatttctttctttgcaccaaagacatcagtctccaacatgtttctgttttgttgt |
| | | ttgattcagcaaaaatctcccagtgacaacatcgcaatagttttttacttctcttaggtggctctgggatgggagaggggtaggatgta |
| | | caggggtagtttgttttagaaccagccgtattttacatgaagctgtataattaattgtcattatttttgttagcaaagattaaatgtgt |
| | | cattggaagccatccctttttttacatttcatacaacagaaaccagaaaagcaatactgtttccattttaaggatatgattaatattat |
| | | taatataataatgatgatgatgatgatgaaaactaaggatttttcaagagatctttctttccaaaacatttctggacagtacctgattg |
| | | t |
| 35 | 201667 | gagtggactattaaatgtgcctaaatgaattttgcagtaactggtattcttgggttttcctacttaatacacagtaattcagaacttgt |
| | _at | attctattatgagtttagcagtcttttggagtgaccagcaactttgatgtttgcactaagattttatttggaatgcaagagaggttgaa |
| | | agaggattcagtagtacacatacaactaatttatttgaactatatgttgaagacatctaccagtttctccaaatgccttttttaaaact |
| | | catcacagaagattggtgaaaatgctgagtatgacacttttcttcttgcatgcatgtcagctacataaacagttttgtacaatgaaaat |
| | | tactaatttgtttgacattccatgttaaactacggtcatgttcagcttcattgcatgtaatgtagacctagtccatcaga |
| 36 | 201739 | tgctgtgtgaaccgtcgtgtgagtgtggtatgcctgatcacagatggattttgttataagcatcaatgtgacacttgcaggacactaca |
| | _at | acgtgggacattgtttgtttcttccatatttggaagataaatttatgtgtagacttttttgtaagatacggttaataactaaaatttat |
| | | tgaaatggtcttgcaatgactcgtattcagatgcctaaagaaagcattgctgctacaaatatttctatttttagaaagggtttttatgg |
| | | accaatgccccagttgtcagtcagagccgttggtgtttttcattgtttaaaatgtcacctgtaaaatgggcattatttatgtttttttt |
| | | tttgcattcctgataattgtatgtattgtataaagaacgtctgtacattgggttataacactagtatatttaaacttacaggcttattt |
| | | gtaatgtaaaccaccattttaatgtactgtaattaacatggttataatacgtacaatccttccctcatcccatcacacaactttttttg |
| 37 | 201743 | ccatccagaatctagcgctgcgcaacacaggaatggagacgcccacaggcgtgtgcgccgcactggcggcggcaggtgtgcagccccac |
| | _at | agcctagacctcagccacaactcgctgcgcgccaccgtaaaccctagcgctccgagatgcatgtggtccagcgccctgaactccctcaa |
| | | tctgtcgttcgctgggctggaacaggtgcctaaaggactgccagccaagctcagagtgctcgatctcagctgcaacagactgaacaggg |
| | | cgccgcagcctgacgagctgcccgaggtggataacctgacactggacgggaatcccttcctggtccctggaactgccctcccccacgag |
| | | ggctcaatgaactccggcgtggtcccagcctgtgcacgttcgaccctgtcggtgggggtgtcgggaaccctggtgctgctccaaggggc |
| | | ccggggctttgcctaagatccaagacagaataatgaatggactcaaactgccttggcttcaggggagtcccgtcaggacgttgaggact |
| | | tttcgaccaattcaacc |
| 38 | 201744 | caaatgatgtgcaaaaccttttactggttgcatggaaatcagccaagttttataatccttaaatcttaatgttcctcaaagcttggatt |
| | _s_at | aaatacatatggatgttactctcttgcaccaaattatcttgatacattcaaatttgtctggtaaaaaaataggtggtagatattgaggc |
| | | caagaatattgcaaaatacatgaagcttcatgcacttaaagaagtatttttagaataagaatttgcatacttacctagtgaaacttttc |
| | | tagaattatttttcactctaagtcatgtatgtttctct |
| 39 | 201842 | tcagcagtatagggaccttccgcacaagctctgtgttaagattgacaataatagtggggccattttcattttagtcttttctaagagtc |
| | _s_at | aaccacaggcatttaagtcagccaaagaatattgttaccttaaagcactattttatttatagatatatctagtgcatctacatctctat |
| | | actgtacactcacccataattcaaacaattacaccatggtataaagtgggcatttaatatgtaaagattcaaagtttgtctttattact |
| | | atatgtaaattagacattaatccactaaactggtcttcttcaagagagctaagtatacactatctggtgaaacttggattctttcctat |
| | | aaaagtgggaccaagcaatgatgatcttctgtggtgcttaaggaaacttactagagctccactaacagtctcataaggaggcagccatc |
| | | ataaccattga |
| 40 | 201852 | gtgccaatcctttgaatgttccacggaaacactggtggacagattctagtgctgagaagaaacacgtttggtttggagagtccatggat |
| | _x_at | ggtggttttcagtttagctacggcaatcctgaacttcctgaagatgtccttgatgtgcagctggcattccttcgacttctctccagccg |
| | | agcttcccagaacatcacatatcactgcaaaaatagcattgcatacatggatcaggccagtggaaatgtaaagaaggccctgaagctga |
| | | tggggtcaaatgaaggtgaattcaaggctgaaggaaatagcaaattcacctacacagttctggaggatggttgcacgaaacacactggg |
| | | gaatggagcaaaacagtctttgaatatcgaacacgcaaggctgtgagactacctattgtagatattgcaccctatgacattggtggtcc |
| | | tgatcaagaatttggtgtggacgttggccct |
| 41 | 201858 | cctggttctggaatcctcagttcaaggttatcctacgcagagagccaggtaccaatgggtgcgctgcaatccagacagtaattctgcaa |
| | _s_at | actgccttgaagaaaaaggaccaatgttcgaactacttccaggtgaatccaacaagatcccccgtctgaggactgacctttttccaaag |
| | | acgagaatccaggacttgaatcgtatcttcccactttctgaggactactctggatcaggcttcggctccggctccggctctggatcagg |
| | | atctgggagtggcttcctaacggaaatggaacaggattaccaactagtagacgaaagtgatgctttccatgacaaccttaggtctcttg |
| | | acaggaatctgccctcagac |
| 42 | 201859 | tatgctttaatgctgttatctatcttattgttcttgaaaatacctgcattttttggtatcatgttcaaccaacatcattatgaaattaa |
| | _at | ttagattcccatggccataaaatggctttaaagaatatatatatatttttaaagtagcttgagaagcaaattggcaggtaatatttcat |
| | | acctaaattaagactctgacttggattgtgaattataatgatatgccccttttcttataaaaacaaaaaaaaaataatgaaacacagtg |
| | | aatttgtagagtgggggtatttgacatattttacagggtggagtgtactatatactattacctttgaatgtgtttgcagagctagtgga |
| | | tgtgtttgtctacaagtatgattgctgttacataacaccccaaattaactcccaaattaaaacacagttgtgctgtcaatacctcatac |
| | | tgctttaccttttttcctggatatctgtgtattttc |
| 43 | 201865 | gttgagtcgtcatcacttttcagtgatgggagagtagatggtgaaatttattagttaatatatcccagaaattagaaaccttaatatgt |
| | _x_at | ggacgtaatctccacagtcaaagaaggatggcacctaaaccaccagtgcccaaagtctgtgtgatgaactttctcttcatacttttttt |
| | | cacagttggctggatgaaattttctagactttctgttggtgtatcccccccctgtatagttaggatagcatttttgatttatgcatgga |
| | | aacctgaaaaaaagtttacaagtgtatatcagaaaagggaagttgtgccttttatagctattactgtctggttttaacaatttccttta |
| | | tatttagtgaactacgcttgctcattttttcttacataattttttattcaagttattgtacagctgtttaagatgggcagctagttcgt |
| | | agctttcccaaataaactctaaacattaatcaatcatctgtgtgaaaatgggttggtgcttctaacctgatggcacttagctatcagaa |
| | | gaccacaaaaattgactcaaatctccagtattcttg |
| 44 | 201893 | tctcctacatccgcattgctgataccaatatcaccagcattcctcaaggtcttcctccttcccttacggaattacatcttgatggcaac |
| | _x_at | aaaatcagcagagttgatgcagctagcctgaaaggactgaataatttggctaagttgggattgagtttcaacagcatctctgctgttga |
| | | caatggctctctggccaacacgcctcatctgagggagcttcacttggacaacaacaagcttaccagagtacctggtgggctggcagagc |
| | | ataagtacatccaggttgtctaccttcataacaacaatatctctgtagttggatcaagtgacttctgcccacctggacacaacaccaaa |
| | | aaggcttcttattcgggtgtgagtcttttcagcaacccggtccagtactgggagatacagccatccaccttcagatgtgtctacgtgcg |
| | | ctctgccattcaactcggaaactataagtaattctcaagaaagccctcatttttataacctgg |
| 45 | 201920 | gtatcaggcttcaattccattatgttttaatgttgtctctgaagatgacttgtgatttttttttcttttttttaaaccatgaagagccg |
| | _at | tttgacagagcatgctctgcgttgttggtttcaccagcttctgccctcacatgcacagggatttaacaacaaaaatataactacaactt |
| | | cccttgtagtctcttatataagtagagtccttggtactctgccctcctgtcagtagtggcaggatctattggcatattcgggagcttct |
| | | tagagggatgaggttctttgaacacagtgaaaatttaaattagtaacttttttgcaagcagtttattgactgttattgctaagaagaag |
| | | taagaaagaaaaagcctgttggcaatcttggttatttctttaagatttctggcagtgtgggatggatgaatgaagtggaatgtgaactt |
| | | tgggcaagttaaatgggacagccttccatgttcatttgtctacctcttaactga |
| 46 | 201957 | gttgtgcctaccactggctggcacaccagggcaatgatttccctgcagaaggaaggaaagaatgtttcacccttgcatccttcttggag |
| | _at | aagctacagcctgtgctcagttgagtggttcacactcagactttggctttatggttttccttcctccttgtctttgccctgaccttgat |
| | | caacaggggtgaaaagaaccaccctgaggtttccatgcctctcccattttagtggtagcattttgtgtctttactccacccttcaccct |
| | | agttccaccaaggttcacacaccagatgtaactgtttttcagctgagttgtatggattaacttcatccactgtaaatacacctgggatg |
| | | ggggtggggttggcttgtttagggagaagcagccagacttgctttgtgaactgaatgtatttttatgcaattttgagtggcctttcaac |
| | | cctaaga |
| 47 | 202007 | tatcagtagctcgtgttatctttttatcaactgcttcccagngtcctaaaacaatagaaattttggattgaaaagttcagcataaggag |
| | _at | tttgagtcagtaaaggatgggataaaggagtcgagatgattcaatgaaaagtatcacaaaaaagagattgatcaacaagagaaataaaa |
| | | aagcccaagaggaagtggtaggggaaggaatttaagaacagcaataagtaaaactcttaagtaactccaaaaagaaaatggtacatttt |
| | | gccaaagaccacttatacttgagaacatggaagaatttgcctgatactctctttggggaaaagagtctctcctcttttcctcaaacccc |
| | | agtacactcagcctctctgccccaccttctcctgactttgtcctcacttgcttctgcagtacattggaacctgaattgaaagaaagtct |
| | | tccttgaataattggagtttgtcttgagaggcaaatatagccccaagaatcacaagattcgaggaccatgtaggtcttttacgtagccc |
| | | aaatccataaattagtctcactttttgtatttatcgtttcatattaaaccctctatatca |
| 48 | 202037 | taacacttggctcttggtacctgtgggttagcatcaagttctccccagggtagaattcaatcagagctccagtttgcatttggatgtgt |
| | _s_at | aaattacagtaatcccatttcccaaacctaaaatctgtttttctcatcagactctgagtaactggttgctgtgtcataacttcatagat |
| | | gcaggaggctcaggtgatctgtttgaggagagcaccctaggcagcctgcagggaataacatactggccgttctgacctgttgccagcag |
| | | atacacaggacatggatgaaattcccgtttcctctagtttcttcctgtagtactcctcttttagatcc |
| 49 | 202069 | cagtcactctaaatggacaccacatgaacctctgtttagaatacctacgtatgtatgcattggtttgcttgtttcttgacagtacattt |
| | _s_at | ttagatctggccttttcttaacaaaatctgtgcaaaagatgcaggtggatgtccctaggtctgttttcaaagaactttttccaagtgct |
| | | tgattatttattaagtgtctacctggtaaatgttttttttgtaaactctgagtggactgtatcatttgctattctaaaccattttacac |
| | | ttaagttaaaatagtttctcttcagctgtaaataacaggatacagaattaacaagagaaaatgtctaactttttaagaaaaaccttatt |
| | | ttcttcggtttttgaaaaacataatggaaataaaacaggatattgacataatagcacaaaatgacactcttctaaaactaaatgggcac |
| | | aagagaattttcctggga |
| 50 | 202133 | gtcaaaacctaattgcaattctgttaaatctaagtaatttttagacagtgtttcaccgtattatttaggatgtgaaatgccatttcttt |
| | _at | cactgattacaccatatacaggaaacaggtaaaacagtgaaaactttattgtgctggttgatgccaacttggttgaaaagctctctgca |
| | | gaagaagtgatctagactgacagaagtgttgctaattacaagttgtgttctcatgacgtaattagaaagtaacttctcaaagtacaact |
| | | tttatgaaaaaaataagctgttaaaaaaaggaaatcgtaggttaatttaattgggaaaatgggcaattgacagagaccattttcctaac |
| | | acatatatgtgctagtactttaactttttaaaattttacttctacgttttgtaatataaaaatttctattttaagtttagaatgttata |
| | | cgtaccgaaagtatgcagccaaatcgatcagatcaaaccattttacctggagtt |
| 51 | 202222 | agctgtgagccttggctgttggcagtgagtgagcctggctcttgtgctggatggagcccaggcgggagcggtggccctgtccctcccac |
| | _s_at | ctctgtgacctgaggcctacgctttggctctggagatagccccagagcagggtgttgggatactgcagggccaggactgagcccc |
| 52 | 202242 | ctgttagctcctcactgtggtaaatgccacacacctttaagtagataagcagacgatagttatctgttcttttgacttaatctcatttg |
| | _at | gtttgattttccctctactaaggctttcctaccttcttcaggctgcctaagacatgtaagcgaaacacttcaataattgtccatgagga |
| | | gaaaaaaagcattgtcatgcatgaaggaaactgaacttgaggtggcctccttgcttgttacatacctgggtatgtgtaggcagtttagt |
| | | gcatctttgcctctcagttgaaacctgtataaccctgttacaaagctgtgttgttgcttcttgtgaaggccatgatattttgttttttc |
| | | cccaattaattgctattgtgttattttactaacttctctctgtattttttcttgcattgacattatagacattgaggacctcatccaaa |
| | | caatttaaaaatgagtgtgaagggggaacaagtcaaaatatttttaaaagatcttcaaaaataatgcctctgtctagcatgccaac |
| 53 | 202266 | tgtcagagttttcaacggtgcttatagctgccagctggattccaaacaggtaccccattgtctctgagctaatgtttatatttttccat |
| | _at | tcaggcaccgaaatagttaatatttaaaataagtcttcaaaagaaaacataagagattattgagttcttgggactggatcattatttca |
| | | taagttcagatcatcttaaatgaaaatgccatgattatctgcagttaagtagatgacagctattctacatcagacttgatttttgtcag |
| | | ctaattacataattggtaagctataattgaaaccttatggataaaattccttaactcctttttgattcatgtttgtagtcatgttgtca |
| | | acagaggcaaagttaagcttgatgatggttaaaatcggtttgatagcaccatgggacatttttctaacaaaaataaatgcatgaagaga |
| | | catagccttttagttttgct |
| 54 | 202274 | aagagctatgagctgccagatgggcaggttatcaccattggcaatgagcgcttccgctgccctgagaccctcttccagccttcctttat |
| | _at | tggcatggagtccgctggaattcatgagacaacctacaattccatcatgaagtgtgacattgacatccgtaaggacttatatgccaaca |
| | | atgtcctctctgggggcaccaccatgtaccctggcattgctgacaggatgcagaaggagatcacagccctggcccccagcaccatgaag |
| | | atcaagattattgctcccccagagcggaagtactcagtctggatcgggggctctatcctggcctctctctccaccttccagcagatgtg |
| | | gatcagcaagcctgagtatgatgaggcagggccctccattgtccacaggaagtgcttctaaagtcagaacaggttctccaaggatcccc |
| 55 | 202283 | caactgcccttgaccggaagcatgagtatcatcttcttcctgcccctgaaagtgacccagaatttgaccttgatagaggagagcctcac |
| | _at | ctccgagttcattcatgacatagaccgagaactgaagaccgtgcaggcggtcctcactgtccccaagctgaagctgagttacgaaggcg |
| | | aagtcaccaagtccctgcaggagatgaagctgcaatccttgtttgattcaccagactttagcaagatcacaggcaaacccatcaagctg |
| | | actcaggtggaacaccgggctggctttgagtggaacgaggatggggcgggaaccacccccagcccagggctgcagcctgcccacctcac |
| | | cttcccgctggactatcaccttaaccagcctttcatcttcgtactgagggacacagacacaggggcccttctcttcattggcaagattc |
| | | tggaccccaggggcccctaatatcccagtttaatattccaataccctagaagaaaacccgagggacagcagattccacag |
| 56 | 202291 | ctgacctgcaggacgaaaccatgaagagcctgatccttcttgccatcctggccgccttagcggtagtaactttgtgttatgaatcacat |
| | _s_at | gaaagcatggaatcttatgaacttaatcccttcattaacaggagaaatgcaaataccttcatatcccctcagcagagatggagagctaa |
| | | agtccaagagaggatccgagaacgctctaagcctgtccacgagctcaatagggaagcctgtgatgactacagactttgcgaacgctacg |
| | | ccatggtttatggatacaatgctgcctataatcgctacttcaggaagcgccgaggggccaaatgagactgagggaagaaaaaaaatctc |
| | | tttttttctggaggctggcacctgattttgtatccccctgtagcagcattactgaaatacataggcttatatacaatgcttctttcct |
| 57 | 202350 | gaagccctggaaaatcgcctgagatacagatgaagattagaaatcgcgacacatttgtagtcattgtatcacggattacaatgaacgca |
| | _s_at | gtgcagagccccaaagctcaggctattgttaaatcaataatgttgtgaagtaaaacaatcagtactgagaaacctggtttgccacagaa |
| | | caaagacaagaagtatacactaacttgtataaatttatctaggaaaaaaatccttcagaattctaagatgaatttaccaggtgagaatg |
| | | aataagctatgcaaggtattttgtaatatactgtggacacaacttgcttctgcctcatcctgccttagtgtgcaatctcatttgactat |
| | | acgataaagtttgcacagtcttacttctgtagaacactggccataggaaatgctgtttttttgtactggactttaccttgatatatgt |
| 58 | 202388 | gctggtatcagaacagcttccctcactgtgtacagaacgcaagaagggaataggtggtctgaacgtggtgtctcactctgaaaagcagg |
| | _at | aatgtaagatgatgaaagagacaatgtaatactgttggtccaaaagcatttaaaatcaatagatctgggattatgtggccttaggtagc |
| | | tgtgttacatctttccctaaatcgatccatgttaccacatagtagttttagtttaggattcagtaacagtgaagtgtttactatgtgca |
| | | agggtattgaagttcttatgaccacagatcatcagtactgttgtctcatgtaatgctaaaactgaaatggtccgtgtttgcattgttaa |
| | | aaatgatgtgtgaaatagaatgagtgctatggtgttgaaaactgcagtgtccgttatgagtgccaaaaatctgtcttgaaggcagctac |
| | | actt |
| 59 | 202403 | aacctgaaaacatcccagccaagaactggtataggagctccaaggacaagaaacacgtctggctaggagaaactatcaatgctggcagc |
| | _s_at | cagtttgaatataatgtagaaggagtgacttccaaggaaatggctacccaacttgccttcatgcgcctgctggccaactatgcctctca |
| | | gaacatcacctaccactgcaagaacagcattgcatacatggatgaggagactggcaacctgaaaaaggctgtcattctacagggctcta |
| | | atgatgttgaacttgttgctgagggcaacagcaggttcacttacactgttcttgtagatggctgctctaaaaagacaaatgaatgggga |
| | | aagacaatcattgaatacaaaacaaataagccatcacgcctgcccttccttgatattgcacctttggacatcggtggtgctgaccagga |
| | | attctttgtggacattggcccagtctgtt |
| 60 | 202555 | gagccattggaagactgtcctctatggcaatgatctcagggctcagtggcaggaaatcctcaacagggtcaccaaccagcccgctcaat |
| | _s_at | gcagaaaaactagaatctgaagatgtgtcccaagctttccttgaggctgttgctgaggaaaagcctcatgtaaaaccctatttctctaa |
| | | gaccattcgcgatttagaagttgtggagggaagtgctgctagatttgactgcaagattgaaggatacccagaccccgaggttgtctggt |
| | | tcaaagatgaccagtcaatcagggagtcccgccacttccagatagactacgatgaggacgggaactgctctttaattattagtgatgtt |
| | | tgcggggatgacgatgccaagtacacctgcaaggctgtcaacagtcttggagaagccacctgcac |
| 61 | 202620 | ttatcaagtgtcaagatcagcaagtgtccttaagtcaaataggtttttttttgttggtggttgtgcttgctttccttttttagaaagtt |
| | _s_at | ctagaaaataggaaaacgaaaaatttcattgagatgagtagtgcatttaattattttttaaaaaactttttaagtacttgaattttata |
| | | tcaggaaaacaaagttgttgagccttgcttcttccgttttgccctttgtctcgctccttattcttttttggggggagggttatttgatt |
| | | tttatcttcctggcataatttccattttattcttctgagtgtctatgttaacttccctctatcccgcttataaaaaaattctccaacaa |
| | | aaatacttgttgacttgatgttttatcacttctctaagtaaggttgaaatatccttattgtagctactgttttaatgtaaaggttaaac |
| | | cttgaaaagaaattcttaatacggtgccaaaattcattttctaacaccatgtgtt |
| 62 | 202686 | agattctaacggtctgttctgtttcaaggcactctagattccattggtccaagattccggatcctaagcatctaagttataagactctc |
| | _s_at | acactcagttgtgactaactagacaccaaagttctaataatttctaatgttggacacctttaggttctttgctnnattctgcctctcta |
| | | ggaccatggttaagagtccaagaatccacatttctaaaatcttatagttctaggcactgtagttctaagactcaaatgttctaagtttc |
| | | taagattctaaaggtccacaggtctagactattaggtgcaatttcaaggttctaaccctatactgtagtattctttggggtgcccctct |
| | | ccttcttagctatcattgcttcctcctccccaactgtgggggtgtgcccccttcaagcctgtgcaatgcattagggatgcctcctttcc |
| | | gcaggggatggacgatctcccacctttcgggccatgttgcccccgtgagccaatccctcaccttctgagtacagagtgtggactctggt |
| | | gcctcca |
| 63 | 202731 | ggctctggaggtgggcagcaatctgtcaatcaccttgttaaagagattgatatgctgctgaaagaatatttactctctggagacatatc |
| | | tgaagctgaacattgccttaaggaactggaagtacctcattttcaccatgagcttgtatatgaagctattataatggttttagagtcaa |
| | _at | ctggagaaagtacatttaagatgattttggatttattaaagtccctttggaagtcttctaccattactgtagaccaaatgaaaagaggt |
| | | tatgagagaatttacaatgaaattccggacattaatctggatgtcccacattcatactctgtgctggagcggtttgtagaagaatgttt |
| | | tcaggctggaataatttggaaacaactcagagatctttgtccttcaaggggcagaaagcgttttgtaagcgaaggagatggaggtcgtc |
| | | ttaaaccagagagctactgaatataagaactcttgcagtcttagatgttat |
| 64 | 202741 | gtaagattcctcctaactttcacagtcgatgacaagattgtctttttatctgatattttgaagggtatattgctttgaagtaagtctca |
| | _at | ataaggcaatatattttagggcatctttcttcttatctctgacagtgttcttaaaattatttgaatatcataagagccttggtgtctgt |
| | | cctaattcctttctcactcaccgatgctgaatacccagttgaatcaaactgtcaacctaccaaaaacgatattgtggcttatgggtatt |
| | | gctgtctcattcttggtatattcttgtgttaactgcccattggcctgaaaatactcattgtaagcctgaaaaaaaaaatctttcccact |
| | | gttttttctgcttgttgtaagaatcaaatgaaataatgtatgtgaaagcaccttgtaaactgtaacctatcaatgtaaaatgttaaggt |
| | | gtgttgttatttcattaattacttctttgtttagaatggaatttcctatgcactactgtagc |
| 65 | 202742 | tacacttttttttgccaactgacttaacaacattgctgtcaggtggaaatttcaagcacttttgcacatttagttcagtgtttgttgag |
| | s_at | aatccatggcttaacccacttgttttgctatttttttctttgcttttaattttccccatctgattttatctctgcgtttcagtgaccta |
| | | ccttaaaacaacacacgagaagagttaaactgggttcattttaatgatcaatttacctgcatataaaatttatttttaatcaagctgat |
| | | cttaatgtatataatcattctatttgctttattatcggtgcaggtaggtcattaacaccacttcttttcatctgtaccacaccctggtg |
| | | aaacctttgaagacataaaaaaaacctgtctgagatgttctttctaccaatctatatgtctttcggttatcaagtgtttctgcatggta |
| | | atgtc |
| 66 | 202746 | aaactactaaccactgcaagctcttgtcaaattttagtttaattggcattgcttgttttttgaaactgaaattacntgagtttcattnc |
| | _at | tttgaatttatagggtttagatttctgaaagcagcatgaatatatcacctaacatcctgacaataaattccatccgttgttttttttgt |
| | | ttgtttgttttttcttttcctttaagtaagctctttattcatcttatggtgcagcaattttaaaatttgaaatattttaaattgttttg |
| | | aactttttgtgtaaaatatatcagatctcaacattgttggtttctttgtttttcattttgtacaactttcttgaatttagaaattacat |
| | | ctttgcagttctgttaggtgctctgtaattaacctgacttatatgtgaacaattttcatgagacagtcatttttaactaatgcagtgat |
| | | tctttctcactactatctgtattgtggaatgcacaaaattgtgtaggtgctgaatgctgtaaggagtttaggttgtatgaattctacaa |
| | | ccctataata |
| 67 | 202760 | gagatactatttttgctgagcaaccagtgtgtttcaggatgatacaaagaaaaatatagaatagaaataagtgcaggcttggaatcagc |
| | _s_at | tacaaatcctaaagatggggtgtgtgtggatgtgtgtgtgtgtgtgtgtacaccattgtgtgtttgtaaaatgtgtatgttcatgagta |
| | _ | agggtgtgtgtgtgtgtgtattaaaattccagagtgaccgtggcacttgggtgtacaggtaattcctccagagctgtttgctggcttca |
| | | ggagtggagtgagaatttcttttttatgaaaagggatataaaggcaccgagctgatgcagtatttgtaatattaagttgacctaacaag |
| | | gtatttgcatgagtcacaattacaaagttttgagcggttttgtaatttgacatttaggaaagtctcctatttattctcatactttacat |
| | | tcatgcttagtatactatagaggatgccagctttaatctttctg |
| 68 | 202766 | gctttcaattgatggactactctattttttgcaaatttgtaaactttgcttctccaaatacaagtactaggttgtccatttatggtacc |
| | _s_at | tatttggtgctagtaaattttcaaactagatttataaatgcactgtaatatgtacacaacttagaaaccaaattacaagtattcagttc |
| | | caatacttcattaatttcaatcaaccaaagttagttcagtagcttatctcagttatgagtataatacattacatgtaaattaagtgtgt |
| | | gtatactgtaatcgtgctattttttatcattgaaacatttataaactagaataataatgcccttaatgtgagggtttgtaatggtgctt |
| | | attaagaccaaagacttgttaaatgtatacaccaagtggtaatgaaatttcgtgactggcccacacgtgcatagaggtctgggaggacc |
| | | aggaaacagcctcagtggccagaggatcaccagtgcatccttcatcacagcatgtgcaatatgccaagattaccctcggtcattcctgt |
| | | caacaaggg |
| 69 | 202768 | gcgtgagtgtgtgagcgcttctgcagcctcggcctaggtcacgttggccctcaaagcgagccgttgaattggaaactgcttctagaaac |
| | __at | tctggctcagcctgtctcgggctgacccttttctgatcgtctcggcccctctgattgttcccgatggtctctctccctctgtcttttct |
| | | cctccgcctgtgtccatctgaccgttttcacttgtctcctttctgactgtccctgccaatgctccagctgtcgtctgactctgggttcg |
| | | ttggggacatgagattttattttttgtgagtgagactgagggatcgtagatttttacaatctgtatctttgacaattctgggtgcgagt |
| | | gtgagagtgtgagcagggcttgctcctgccaaccacaattcaatgaatccccgacccccctaccccatgctgtacttgtggttctcttt |
| | | ttgtattttgcatctgaccccggggggctgggacagattggcaatgggccgtccc |
| 70 | 202838 | agaaagaggcgctgctcactgttttcctgcttcagtttttctcttatagtaccatcactataatcaacgaacttctcttctccacccag |
| | _at | agatggcttttccaacacattttaattaaaggaactgagtacattaccctgatgtctaaatggaccaaagatctgagatccattgtgat |
| | | tatatctgtatcaggtcagcagaagaaggaactgagcagttgaactctgagttcatcaattctaatatttggaaattatctacaatgga |
| | | atcttccctctgttctctgataacctacttgcttactcaatgcctttaagccaagtcaccctgttgcctatgggaggaggtggaaggat |
| | | ttggcaagctcaaccacatgctatttagttagcatcagttgtcaccaacagtctttctgcaaagggcaggagagctttgggggaaagga |
| | | aaaggcttaccaggctgctatggtcaactcttcagaa |
| 71 | 202888 | agccctggagaagacgaaagccaacatcaagtgggtgaaggagaacaaggaggtggtgctccagtggttcacagaaaacagcaaatagt |
| | _s_at | ccccagcccttgaagtcacccggccccgatgcaaggtgcccacatgtgtccatcccagcggctggtgcagggcctccattcctggagcc |
| | | cgaggcaccagtgtcctcccctcaaggacaaagtctccagcccacgttctctctgcctgtgagccagtctagttcctgatgacccaggc |
| | | tgcctgagcacctcccagcccctgcccctcatgccaaccccgccctaggcctggcatggcacctgtcgcccagtgccctggggctgatc |
| | | tcagggaagcccagctccagggccagatgagcagaagctctcgatggacaatgaacggccttgctgggggccgccctgtaccctctttc |
| | | acctttccctaaagaccctaaatctgaggaatcaacagggcagcagatctgtatatttttttc |
| 72 | 202920 | attatctggtcttttcctgttgtgcaaaaatgactcattgctccgaatgtcaaaaacaaatgcgacaaacaatggcacttcatcattta |
| | _at | aagtaatgttgccaagagaaaaaatttcctgggagggaggtttcccacaagccaaatctcctaagcctcaaatgctagcactttttggc |
| | | agttggataggaaatgagacattctttggcagccaaaataagagaggccgatggtgaaactttttgagacaccctatggccttcttgtc |
| | | aaaaccttcactggagctcaagaaaagcatttctgttgtgttatttgcagtgcagatgatgtctgtgtaacaacataatggttattcac |
| | | ctttttttgattttgatttttgctgtgttatcaaaaacttgaatactgtgagaagaagtgaattttcagttgacgaatcagcatcttgt |
| | | tcccatggtgataac |
| 73 | 202953 | cttcgaccacgtgatcaccaacatgaacaacaattatgagccccgcagtggcaagttcacctgcaaggtgcccggtctctactacttca |
| | _at | cctaccacgccagctctcgagggaacctgtgcgtgaacctcatgcgtggccgggagcgtgcacagaaggtggtcaccttctgtgactat |
| | | gcctacaacaccttccaggtcaccaccggtggcatggtcctcaagctggagcagggggagaacgtcttcctgcaggccaccgacaagaa |
| | | ctcactactgggcatggagggtgccaacagcatcttttccgggttcctgctctttccagatatggaggcctgacctgtgggctgcttca |
| | | catccaccccggctccccctgccagcaacgctcactctacccccaacaccaccccttgcccagccaatggacacagtagggcttggtga |
| | | atgctgctgagtgaatgagtaaataaactcttcaaggccaaggaacagtggtctaattcaactctgtgtcccagcactggcacaccaga |
| | | agtgccatgctcagaaa |
| 74 | 202957 | tgatgagctttcctttgatccggacgacgtaatcactgacattgagatggtggacgagggctggtggcggggacgttgccatggccact |
| | _at | ttggactcttccctgcaaattatgtcaagcttctggagtgactagagctcactgtctactgcaactgtgatttcccatgtccaaagtgg |
| | | ctctgctccaccccctccctattcctgatgcaaatgtctaaccagatgagtttctggacagacttccctctcctgcttcattaagggct |
| | | tggggcagagacagcatggggaaggaggtccccttccccaagagtcctctctatcctggatgagctcatgaacatttctcttgtgttcc |
| | | tgactccttcccaatgaacacctctctgccaccccaagctctgctctcctcctctgtgagctctgggcttcccagtttgtttacccggg |
| | | aaagtacgtctagattgtgtggtttgcctcattgtgctatttgcccactttccttccctgaagaaatatctgtgaaccttctttctgtt |
| | | cagtccta |
| 75 | 202992 | gctggaatacttactcttgtcgggagattgaaccactaaaatgttagagcagaattcattatgctgtggtcacaggggtgtcttgtctg |
| | _at | agaacaaatacaattcagtcttctctttggggttttagtatgtgtcaaacataggactggaagtttgcccctgttcttttttcttttga |
| | | aagaacatcagttcatgcctgaggcatgagtgactgtgcatttgagatagttttccctattctgtggatacagtcccagagttttcagg |
| | | gagtacacaggtagattagtttgaagcattgaccttttatttattccttatttctctttcatcaaaacaaaacagcagctgtgggagga |
| | | gaaatgagagggcttaaatgaaatttaaaataagctatattatacaaatactatctctgtattgttctgaccctggtaaa |
| 76 | 202994 | aatgcgaaggctaagtgtcaccccctttctctgcctctggctgggccttgctaagggccaaggaaagaaagacattttttagggggcag |
| | _s_at | ccagtccaaatgccaaaagaagaccagttcttgccctgattgtatgaaatttgacattttggcactttttttttattttnggccaatca |
| | | gattttctatgttctaaggacatggctgctgtagaatagcacagacgtggatgataaattatccccagaagcagcatgacagaatgcct |
| | | cggggagcacttggaagggaaattgcagttctgttgaaatagaggaaaatcccttggtaaagacacagcctgttaggctcgtgtgggcc |
| | | tccagtatgttcaccaggggaa |
| 77 | 202995 | aacgatgtcacatgcgtgttcgaccccgtgcacaccatctcccacaccgtcatctcgctgcctaccttccgcgagttcacccgccctga |
| | _s_c | agagatcatcttcctccgggccatcacgccaccgcatcctgccagccaggctaacatcatcttcgacatcacggaagggaacctgcggg |
| | | actcttttgacatcatcaagcgttacatggacggcatgaccgtgggtgtcgtgcgccaggtgcggcccatcgtgggcccatttcatgcc |
| | | gtcctgaagctggagatgaactatgtggtcgggggcgtggtttcccaccgaaatgttgtcaacgtccgcatcttcgtctctgagtactg |
| | | gttctgagggctggtctgccgcacagccgcaggtgcacctccaggccaaatcattgctgccagtgactgtggtctgtacttgtttatac |
| | | cctcag |
| 78 | 203000 | gacagacctgagaccaatctgggtagaagcaaaaagttgaaccttttaaagtgctgaacacaaatccaaattcgaatggttcaagcagc |
| | _at | cgtgaaatcgctcttcataaagtgggcttaattctctagtttaagttcttttgatggaatgaattaattaatgtgtcaggtggcttatt |
| | | tgtggatgccatgattgatgatgttcattttaagctcttacctatagtacaagtacatgatgctactgaatatttttccacttggaaac |
| | | tgtgagctggttgcattaaaacacacatacaaacaaaatcaaaaacactgcggactttcactcaagctggtctttcttccccagtgtaa |
| | | ggcaatcctgcctactaacaacaccaacaacaaaacactccatctgtgaanntgacgcagttaagggggctaggcagggcatttgtgcc |
| | | aactaagaatcaccagatacccaccataagtacctatcgcagttttgaagtc |
| 79 | 203001 | acctcgcaacatcaacatctatacttacgatgatatggaagtgaagcaaatcaacaaacgtgcctctggccaggcttttgagctgatct |
| | _s_at | tgaagccaccatctcctatctcagaagccccacgaactttagcttctccaaagaagaaagacctgtccctggaggagatccagaagaaa |
| | | ctggaggctgcaggggaaagaagaaagtctcaggaggcccaggtgctgaaacaattggcagagaagagggaacacgagcgagaagtcct |
| | | tcagaaggctttggaggagaacaacaacttcagcaagatggcggaggaaaagctgatcctgaaaatggaacaaattaaggaaaaccgtg |
| | | aggctaatctagctgctattattgaacgtctgcaggaaaaggagaggcatgctgaggaggtgcgcaggaacaaggaactccaggttgaa |
| | | ctgtctggctgaagcaagggagggtctggcacgcc |
| 80 | 203058 | atggcctctgtgaataatgtaactccagttacacggtgacttttaatagcatacagtgatttgatgaaaggacgtcaaacaatgtggcg |
| | _s_at | atgtcgtggaaagttatctttcccgctctttgctgtggtcattgtgtcttgcagaaaggatggccctgatgcagcagcagcgccagctg |
| | | taataaaaaataattcacactatcagactagcaaggcactagaactggaaaagaccacagaaaacaaagaatccaaccctttcatctta |
| | | caggtgaacaaactgtgatgatgcacatgtatgtgttttgtaagctgtgagcaccgtaacaaaatgtaaatttgccattattaggaaag |
| | | tgctggtggcagtgaagaagcacccaggccacttgactcccagtctggtgccctgtctacaccagacaacacaggagctgggtcagatt |
| | | cccctcagctgcttaacaaagttcctcgaacagaaagtgcttacaaagctgccttctcggatact |
| 81 | 203060 | agctgccttctcggatactgaaaggtcgagttttctgaactgcactgattttattgcagttgaaaaacccaaagctattccaaagattt |
| | _s_at | caagctgttctgagacatcttctgatggctttacttcctgagaggcaatgtttttactttatgcataattcattgttgccaaggaataa |
| | | agtgaagaaacagcacctttttaatatataggtctctctggaagagacctaaatttagaaagagaaaactgtgacaattttcatattct |
| | | cattcttaaaaaacactaatcttaactaacaaaagttcttttgagaataagttacacacaatggccacagcagtttgtctttaatagta |
| | | tagtgcctatactcatgtaatcggttactcactactgcctttaaaaaaaaccagcatatttattgaaaacatgagacaggattatagtg |
| | | ccttaaccgatatattttgtgacttaaaaaatacatttaaaactgctcttctgctctagtaccatgcttagtgcaaatgattatttcta |
| | | tgtacaactgatgcttgttcttattt |
| 82 | 203066 | ttcagttgctaatgaggctcctccgctctggacacaacccttttatagattaatttctctgccaattaacttgtcattttcagtacata |
| | _at | ttttactattccacaccaaccataattacaacaagggatttttcttatgcactcctatgcatgtgaataacatgtggtgtaattctgct |
| | | tcttacagaagtattactgaaggtattatttccaatattatttggtttattatgcggatcttttttatatatgcagtcccatcccttct |
| | | gtgccactcaatgccatccagacatggtttttccctccaggggccatctctccagagggcacttcggctgcctctgcttcctctcattc |
| | | gaggcccggctcttgctgacagaataggttccgttctgggcggtggttctcgagcctgccattcaaaaccaaagcaaattggagcattt |
| | | ctcacaacatggtattgaagttcctttttgttctcaaaagttgtgaccgtgttaaattgtactcccttagtcctgtaaggtatgttaag |
| | | tgaatcgcagttacgctgtactttta |
| 83 | 203131 | agaaaatttgccaatctttcctactttctatttttatgatgacaatcaaagccggcctgagaaacactatttgtgactttttaangatt |
| | _at | agtgatgtccttaaaatgtggtctgccaatctgtacaaaatggtcctatttttgtgaagagggacataagataaaatgatgttatacat |
| | | caatatgtatatatgtatttctatatagacttggagaatactgccaaaacatttatgacaagctgtatcactgccttcgtttatatttt |
| | | tttaactgtgataatccccacaggcacattaactgttgcacttttgaatgtccaaaatttatattttagaaataataaaaagaaagata |
| | | cttacatgttcccaaaacaatggtgtggtgaatgtgtgagaaaaactaacttgatagggtctaccaatacaaaatgtattacgaatgcc |
| | | cctgttcatgtt |
| 84 | 203240 | ccaggactacagaataccatcccctggtaccgtgtagttgccgaagtccagatctgccatggcaaaacggaggctgtgggccaggtcca |
| | _at | catcttcttccaggatgggatggtgacgttgactccaaacaagggtgtgtgggtgaatggtctccgagtggatctcccagctgagaagt |
| | | tagcatctgtgtccgtgagtcgtacacctgatggctccctgctagtccgccagaaggcaggggtccaggtgtggcttggagccaatggg |
| | | aaggtggctgtgattgtcagcaatgaccatgctgggaaactgtgtggggcctgtggaaactttgacggggaccagaccaatgattggca |
| | | tgactcccaggagaagccagcgatggagaaatggagagcgcaggacttctccccatgttatggctgatcagtcatccaccaggaacgaa |
| | | gatttcctgaagaagacctggtccctctggaggttgcggtggctgaaggatgcatcatgtgctcctaccctgctctaccgcttttctgg |
| | | gtcacagag |
| 85 | 203296 | aaagacaagcattgggtcagacccataaaccacctcccaaaggctgtcatttcattgcactggaattttgctttatcagaagcaaggaa |
| | _s_at | gtaagggagtcattgccttgggcctgggaatctaagtgggagacaatattaatttggatccgattaattggagattactaactgtggac |
| | | aaaagtttatctttgcacaatcaataaaaatggcatttttttagtaaattaagagcataaacaatattgctagaggtggcatgtttagt |
| | | ctaccaaaaacaatacttttcaggcactttagaaatatccttttagaagcagcgagtgcatgggctaattatcatcaatctttatgtat |
| | | ttgttaaagaaacatctacaggatctttattggtgaccttttgta |
| 86 | 203305 | gtccttcacatcaccattttgagacctcagcttggcactcaggtgctgaagggtaatatggactcagccttgcaaatagccagtgctag |
| | _at | ttctgacccaaccacagaggatgctgacatcatttgtattatgttccaaggctactacagagaaggctgcctgctatgtatttgcaagg |
| | | ctgatttatggtcagaatttccctctgatatgtctagggtgtgatttaggtcagtagactgtgattcttagcaaaaaatgaacagtgat |
| | | aagtatactgggggcaaaatcagaatggaatgctctggtctatataaccacatttctgagcctttgagactgttcctgagccttcagca |
| | | ctaacctatgagggtgagctggtcccctctatatatacatcatacttaactttactaagtaatctcacagcatttgccaagtctcccaa |
| | | tatccaatt |
| 87 | 203343 | atgtgtcactgattttttagctcaaaatcatcactgttaatttccagtcaccccaaatatggttaaaagatttttttttttaatcatga |
| | _at | agagaaaattagtagcatttctttctctccccattatttattggttttcctcactaatctttttttttttagtccaaaagccaaaaata |
| | | tttatcttggttttacattttaatttccattcttaattgtaatttttttctttaaataaggaaaccaatataatctcatgtataaaaac |
| | | ttaaatattttacaagttacatatagcatcattctaaaataagaattttttttgttttctgtctgcttttttcttatgtctcttgttga |
| | | gttttatattttcagtggttatttttgcttgtgttagatcattattaaaatatatccaatgtccctttgatacttgtgctctgctgaga |
| | | atgtacagtttgcattaaacatcccaggtctcatccttcaggaattt |
| 88 | 203382 | gaagcgcctggcagtgtaccaggccggggcccgcgagggcgccgagcgcgggttctcagcgccatccgcgagcgcctggggcccctggt |
| | s_at | ggaacagggccgcgtgcgggccgccactgtgggctccctggccggccagccgctacaggagcgggcccaggcctggggcgagcggctgc |
| | | gcgcgcggatggaggagatgggcagccggacccgcgaccgcctggacgaggtgaaggagcaggtggcggaggtgcgcgccaagctggag |
| | | gagcaggcccagcagatacgcctgcaggccgaggccttccaggcccgcctcaagagctggttcgagcccctggtggaagacatgcagcg |
| | | ccagtgggccgggctggtggagaaggtgcaggctgccgtgggcaccagcgccgcccctgtgcccagcgacaatcactgaacgccgaagc |
| | | c |
| 89 | 203474 | actgtgatataggtactctgatttaaaactttggacatcctgtgatctgattaaagttggggggtgggaaatttagctgactagggaca |
| | _at | aacatgtaaacctattttcctatgaaaaaagttttaaatgtcccacttgaataacgtaattcttcatagtttttttaatctatggataa |
| | | atggaaacctaattatttgtaatgaattatttagacagttctaagccctgtcttctgggagttatcaattttaaagagaacttttgtgc |
| | | aattcaaatgaagtttttataagtaattgaaaatgacaacacaataacactttctgtataaaagtatatattttatgtgatttattcct |
| | | actaaatgaaagtgcactactgcctcatgtaaagactcttgcacgcagagcctttaagtgactaaggaacaacatagatagtgagcata |
| | | gtccccacctccacccctcacaatttatttgaatacttcaattgtgcctctcaa |
| 90 | 203477 | ttcagacagttatatcctccttttaaaccattgttgttgagtgtaagatgtccttcatgttttcttataaagtcagtgtttagaaatgt |
| | _at | taccctttctaagttatatacagatcaaatgcttttttctttcacgtacatccatcatttgcaactgctgttcgtacacagaaacagga |
| | | ctgctcaaatgatcctatttgtattttctgatgctatcagactctaatgtttttttccctaaaatattattgccatcatgctttaggaa |
| | | tttttatatttttacacaatcatattttagtatggtgtctgtttatgtaactctgacttgctggaaaagttgaaactccaaataatctg |
| | | aaactagaaaagaaatagcacataattactaccttccccttggcggctctcctcccccaacccccaccccacaattttatgacttcca |
| 91 | 203638 | caacgtctaactggacttcccaagataaatggtaccagcgtcctcttaaaagatgccttaatccattccttgaggacagaccttagttg |
| | _s_at | aaatgatagcagaatgtgcttctctctggcagctggccttctgcttctgagttgcacattaatcagattagcctgattctcttcagtga |
| | | attttgataatggcttccagactctttgcgttggagacgcctgttaggatcttcaagtcccatcatagaaaattgaaacacagagttgt |
| | | tctgctgatagttttggggatacgtccatctttttaagggattgctttcatctaattctggcaggacctcaccaaaagatccagcctca |
| | | tacctacatcagacaaaatatcgccgttgttccttctgtactaaagtattgtgttttgctttggaaacacccactcact |
| 92 | 203645 | gccagacgctggggccatagtgagtgtgggcacaaggaagacgctgcagtgaattgcacagatatttcagtgcagaaaaccccacaaaa |
| | _s_at | agccacaacaggtcgctcatcccgtcagtcatcctttattgcagtcgggatccttggggttgttctgttggccattttcgtcgcattat |
| | | tcttcttgactaaaaagcgaagacagagacagcggcttgcagtttcctcaagaggagagaacttagtccaccaaattcaataccgggag |
| | | atgaattcttgcctgaatgcagatgatctggacctaatgaattcctcaggaggccattctgagccacactgaaaaggaaaatgggaatt |
| | | tataacccagtgagttcagcctttaagataccttgatgaagacctggactattgaatggagcagaaattcacctctctcactgactatt |
| | | acagttgcatttttatggagttcttcttctcctaggattcc |
| 93 | 203680 | ttttcattgttcattgatatgctcagtatgctgccacataagatgaatttaattatattcaaccaaagcaatatactcttacatgattt |
| | _at | ctaggccccatgacccagtgtctagagacattaattctaaccagttgtttgcttttaaatgagtgatttcattttgggaaacaggtttc |
| | | aaatgaatatatatacatgggtaaaattactctgtgctagtgtagtcttactagagaatgtttatggtcccacttgtatatgaaaatgt |
| | | ggttagaatgttaattggataatgtatatataagaagttaaagtatgtaaagtataacttcagccacatttttagaacactgtttaaca |
| | | tttttgcaaaaccttcttgtaggaaaagagagctctctacatgaagatgacttgttttatatttcagattttattaaaagccatgtctg |
| | | ttaaacaagaaaaaacacaaaagaactccagattcctggttcatcattctgtattcttactcacttatcaagtt |
| 94 | 203729 | atcctcattcttatactgatttcgtggccactttggacaagtcctggtggactctccctgggaaagagtccctgaatctctggtacgac |
| | _at | tgcacgtggaacaacgacaccaaaacatgggcctgcagtaatgtcagcgagaatggctggctgaaggcggtgcaggtcctcatggtgct |
| | | ctccctcattctctgctgtctctccttcatcctgttcatgttccagctctacaccatgcgacgaggaggtctcttctatgccaccggcc |
| | | tctgccagctttgcaccagcgtggcggtgtttactggcgccttgatctatgccattcacgccgaggagatcctggagaagcacccgcga |
| | | gggggcagcttcggatactgcttcgccctggcctgggtggccttccccctcgccctggtcagcggcatcatctacatccacctacggaa |
| | | gcgggagtgagcgccccgc |
| 95 | 203748 | caagtatcggggctctgctatcaaggtgcaaagtccttcgtggatgcaacctcaaccatatattctacagcaccctggtgccgtgttaa |
| | _x_at | ctccctcaatggagcacaccatgtcactacagcccgcatcaatgatcagccctctggcccagcagatgagtcatctgtcactaggcagc |
| | | accggaacatacatgcctgcaacgtcagctatgcaaggagcctacttgccacagtatgcacatatgcagacgacagcggttcctgttga |
| | | ggaggcaagtggtcaacagcaggtggctgtcgagacgtctaatgaccattctccatatacctttcaacctaataagtaactgtgagatg |
| | | tacagaaaggtgttcttacatgaagaagggtgtgaaggctgaacaatcatggatttttctgatcaattgtgctttaggaaattattgaa |
| | | cagttttgcacaggttcttgaaaacgttatttataatgaaatcaactaaaactatttttgctataagttctataaggtgcataaaaccc |
| | | ttaattcatctagtagctgttccccc |
| 96 | 203766 | caccttgggtctgtgtcacttgtaggtttctctgcctccaggttgcctcaacagcaggaggcacagcagtttcaccatctttgaggtga |
| | _s_a | gggtggggtgccccagctaggaagcaagatcgctgtgctaggtctgaccaaaaccagagggcagtctagtcctgggggtaaagccctca |
| | | gatcccagggtacactcttctccattccctccacccacttgcctgtcaccccagtcacctaagcaatcactgggcccagaggagaggag |
| | | acagacacacactggctcctggacctaaagggtatgagctggagctaaggccagctagagcttccactgtcagccctcactgtcagccc |
| | | cactgcacccccctgtgcctgctgggcactgggcactagctagatgctttaggttgcttcagctgatccttcaactctgtgaggtg |
| 97 | 203881 | tatgtgacgctggaccttttctttacccaaggatttttaaaactcagatttaaaacaaggggttactttacatcctactaagaagttta |
| | _s_at | agtaagtaagtttcattctaaaatcagaggtaaatagagtgcataaataattttgttttaatctttttgtttttcttttagacacatta |
| | | gctctggagtgagtctgtcataatatttgaacaaaaattgagagctttattgctgcattttaagcataattaatttggacattatttcg |
| | | tgttgtgttctttataaccaccgagtattaaactgtaaatcataatgtaactgaagcataaacatcacatggcatgttgtcattgtttt |
| | | caggtactgagttcttacttgagtatcataatatattgtgttttaacaccaacactgtaacatttacgaattatttttttaaacttcag |
| | | ttttactgcattttcacaacatatcagacttcaccaa |
| 98 | 203908 | tgtgtaatacgccaaccagtcaagttgtgttttggccagagatttagatatgtccaatttcctggctcatttcattgtgctctatgggt |
| | _at | acgtataaaaagcaagaattctgtttcctaggcaaacattgcaactcagggctaaagtcatccagtgaaacttttagagccagaagtaa |
| | | ctttgtcccagtcctacaatgtgaaaagagtgaatagttgcctctttttagccattttcatggctggtacatattcgtacgcattactt |
| | | ttcagaatcaatacgcactttcagatattcttatttttattctcttaagtctttattaactttggagagagaaatgatgcatctttttt |
| | | attttaaatgaagtagatcaacatggtggaacaaaatgataaagaacagaaaacatttcaatatattactaataactttttccaatata |
| | | aatcctaaaattcctataacatagtattttacagttttatgaagctttctattgtgacttttatg |
| 99 | 203913 | aacactcacatacaagttcatactttaaaagaggaaagctacttaacaatgacaaatatttcacaataataatttttacttatatacca |
| | _s_at | tctttcaactgaacatttcagttcttccaagagcttcttagagtagtatattttgggggcagtcaaggaataaactacagtgtaaacat |
| | | atcccagatgaaaactgctgtatggaaaaatgacagaaagtaactgattgacactgttgattcacagttcangcctcctatctgggaaa |
| | | gacatttctttcctctgctcactttaagaacttttaccgactccaaaaatctcaggaattaaacttttaacagttacagcaataaagaa |
| | | tagttagtactccaaaaatattatatttaagatgctcaacaagaaaaaaatgcaaatgtaatatttttttcaaattacttctttattga |
| | | cttgtcca |
| 100 | 203914 | gatttccaaatcagtcgttttctcaaaaaaatatcgtataagtgactcatcctgtctgctaactccagacctcccagcttgaagccaaa |
| | _x_at | tctttccatgtgagattgatatggatttcctagaagtactggaatgttgtcatatcttgccctattttaattctgctatagaaaacaat |
| | | tgccttcacttttaaggagtaatttgaatattaataactctggtctagattttcatataatgtattaaagacaaagtagtgaacatcaa |
| | | tgaacatctgatagagataaactgtaatcaggcataagcttgtttgtatgttctggcagtgactaatcagtaaatgatgtcggtttgcc |
| | | cagtatcacttatcttctgtatttttcctctgtcgtgtaaatagtataaccttttcatttatggacaattttttggactagtagccttc |
| | | aatatacattctgctt |
| 101 | 203951 | acgcacacaactactacaattccgcctaggtccacaaggccttcactgttttccccccaagggaggctgctgctgctcttggctggagc |
| | _at | cagccagggccagccgaccccctctccctgcatggcatcctccagcccctgtagaactcaacctctacagggttagagtttggagagag |
| | | cagactggcggggggcccattggggggaaggggaccctccgctctgtagtgctacagggtccaacatagaacagggtgtccccaacagc |
| | | gcccaaaggacgcactgagcaacgctattccagctgtccccccactccctcacaagtgggtacccccaggaccagaagctcccccagca |
| | | aagcccccagagcccaggctcggcctgcccccaccccattcccgcagtgggagcaaactgcatgcccagagacccagcggacacacgcg |
| | | gtttggtttgcagcgactggcatac |
| 102 | 203963 | ccaaatttcatttcagccacttctgcaggatccctactgccaacctggaatggagacttttatctacttctctctctctgaagatgtca |
| | _at | aatcgtggtttagatcaaatatatttcaagctataaaagcaggaggttatctgtgcagggggctggcatcatgtatttaggggcaagta |
| | | ataatggaatgctactaagatactccatattcttccccgaatcacacagacagtttctgacaggcgcaactcctccattttcctcccgc |
| | | aggtgagaaccctgtggagatgagtcagtgccatgactgagaaggaaccgacccctagttgagagcaccttgcagttccccgagaactt |
| | | tctgattcacagtctcattttgacagcatgaaatgtatcttgaagcatagctttttaaatatctttttccttctactcctccctctgac |
| | | tctaagaattctctcttctggaatcgcttg |
| 103 | 203980 | gtgtgatgcttttgtaggtacctggaaacttgtctccagtgaaaactttgatgattatatgaaagaagtaggagtgggctttgccacca |
| | _at | ggaaagtggctggcatggccaaacctaacatgatcatcagtgtgaatggggatgtgatcaccattaaatctgaaagtacctttaaaaat |
| | | actgagatttccttcatactgggccaggaatttgacgaagtcactgcagatgacaggaaagtcaagagcaccataaccttagatggggg |
| | | tgtcctggtacatgtgcagaaatgggatggaaaatcaaccaccataaagagaaaacgagaggatgataaactggtggtggaatgcgtca |
| | | tgaaaggcgtcacttccacgagagtttatgagagagcataagccaagggacgttgacctggactgaagttcgcattgaactctacaaca |
| | | ttctgtgggatatattgttcaaaaagatattgttgttttccctgatttagcaagca |
| 104 | 204018 | ggtccccacagactcagagagaacccaccatggtgctgtctcctgccgacaagaccaacgtcaaggccgcctggggtaaggtcggcgcg |
| | _x_at | cacgctggcgagtatggtgcggaggccctggagaggatgttcctgtccttccccaccaccaagacctacttcccgcacttcgacctgag |
| | | ccacggctctgcccaggttaagggccacggcaagaaggtggccgacgccctgaccaacgccgtggcgcacgtggacgacatgcccaacg |
| | | cgctgtccgccctgagcgacctgcacgcgcacaagcttcgggtggacccggtcaacttcaagctcctaagccactgcctgctggtgacc |
| | | ctggccgcccacctccccgccgagttcacccctgcggtgcacgcctccctggacaagttcctggcttctgtgagcaccgtgctgacctc |
| | | caaataccgttaagctg |
| 105 | 204034 | gcccaggctgacttacacattgaggatggagactccatccgcttcgggcgcttcgcgttggagaccagggccagccctggccacacccc |
| | _at | aggctgtgtcaccttcgtcctgaatgaccacagcatggccttcactggagatgccctgttgatccgtgggtgtgggcggacagacttcc |
| | | agcaaggctgtgccaagaccttgtaccactcggtccatgaaaagatcttcacacttccaggagactgtctgatctaccctgctcacgat |
| | | taccatgggttcacagtgtccaccgtggaggaggagaggactctgaaccctcggctcaccctcagctgtgaggagtttgtcaaaatcat |
| | | gggcaacctgaacttgcctaaacctcagcagatagactttgctgttccagccaacatgcgctgtggggtgcagacacccactgcctgat |
| | | ctcacttctgtcagatgacccatccactattaatgcactaggtgggaggagagggcggcaatgacactgcacct |
| 106 | 204036 | aattgccacattttcttatggcattaaaaattttacaaaaacataattttaatggctatattatattccatttaatggatgcaactcan |
| | _at | gtttatttaaccattcccatngttgttaactatttaggttgtttctaattttcattattataaagttgcagaaatttggtgtacataaa |
| | | actgtctccatataattgattattaggatatattcccatgaaggattcntttttttaaaaaaatgtgaaatntcatcttgtacttacac |
| | | ctttcatgnaaagggatttcctgcttagtactgcatgggtggcagttgtgaggaaaagccagtcaaatgacctttttacaaaagaaatg |
| | | cagtggtcacttcagttgagagtgactttttaatacaacaagatcaactagaagaattcaactgtctcaagaatcaaggtaccccaata |
| | | tatctcgcaattccaaactttgtttgagggactcgttatccagctcttggtagccacacctgcaatgta |
| 107 | 204069 | aagccttacagttatcctgcaagggacaggaaggtctgatttgcaggatttttagagcattaaaataactatcaggcagaagaatcttt |
| | _at | cttctcgcctaggatttcagccatgccccgctctctctctttctctctcttttcctctctctccctctttctagcctggggcttgaatt |
| | | tgcatgtctaattcatttactcaccatatttgaattggcctgaacagatgtaaatcgggaaggatgggaaaaactgcagtcatcaacaa |
| | | tgattaatcagctgttgcaggcagtgtcttaaggagactggtaggaggaggcatggaaaccaaaaggccgtgtgtttagaagcctaatt |
| | | gtcacatcaagcatcattgtccccatgcaacaaccaccaccttatacatcacttcctgttttaagcagctctaaaacatagactgaaga |
| | | tttatttttaatatgttgactttatttctgagcaaagcatcggtcatgtgtgtattttttcatagtcccaccttggagcatttatg |
| 108 | 204083 | gaaggcggctgatgagagcgagagaggaatgaaggtcatcgaaaaccgggccatgaaggatgaggagaagatggaactgcaggagatgc |
| | _s_at | agctgaaggaggccaagcacatcgctgaggattcagaccgcaaatatgaagaggtggccaggaagctggtgatcctggaaggagagctg |
| | | gagcgctcggaggagagggctgaggtggccgagagccgagccagacagctggaggaggaacttcgaaccatggaccaggccctcaagtc |
| | | cctgatggcctcagaggaggagtattccaccaaagaagataaatatgaagaggagatcaaactgttggaggagaagctgaaggaggctg |
| | | agacccgagcagagtttgccgagaggtctgtggcaaagttggagaaaaccatcgatgac |
| 109 | 204122 | catggggggacttgaaccctgcagcaggctcctgctcctgcctctcctgctggctgtaagtggtctccgtcctgtccaggcccaggccc |
| | _at | agagcgattgcagttgctctacggtgagcccgggcgtgctggcagggatcgtgatgggagacctggtgctgacagtgctcattgccctg |
| | | gccgtgtacttcctgggccggctggtccctcgggggcgaggggctgcggaggcagcgacccggaaacagcgtatcactgagaccgagtc |
| | | gccttatcaggagctccagggtcagaggtcggatgtctacagcgacctcaacacacagaggccgtattacaaatgagcccgaatcatga |
| | | cagtcagcaa |
| 110 | 204130 | tccccagcagtggctcggtgagccatgtgcacctatggcccagccactgcagcacaggaggctccgtgagccttggttcctccccgaaa |
| | _at | acccccagcattacgatcccccaagtgtcctggaccctggcctaaagaatcccacccccacttcatgcccactgccgatgcccaatcca |
| | | ggcccggtgaggccaaggtttcccagtgagcctctgcgcctctccactgtttcatgagcccaaacaccctcctggcacaacgctctacc |
| | | ctgcagcttggagaactccgctggatgggagtctcatgcaagacttcactgcagcctttcacaggactctgcagatagtgcctctgcaa |
| | | actaaggagtgactaggtgggttggggaccccctcaggattgatctcggcaccagtgcctcagtgctgcaattgagggctaaatcccaa |
| | | gtgtctcttgactggctcaagaattagggcccca |
| 111 | 204135 | cagaccggcagtcatcatggcagtttcagcgttcaaacagcaatagctcaagtgtgataactactgaggataataaaatccacattcac |
| | _at | ttaggaagtccttacatgcaagctgtagccagcccttcagcaccactgcaggataaccgaactcaaggcttaattaacggggcactaaa |
| | | caaaacaaccaataaagtcaccagcagtattactatcacaccaacagccacacctcttcctcgacaatcacaaattacagtaagtaata |
| | | tatataactgaccacgctcaccctcatccagtccatactgatatttttgcaaggaactcaatcctatttaatcatccctccatatcccc |
| | | caagactgactgaactcgtactagggaaggtttgtgcatgaactatacaagagtatctgaaactaactgttgcctgcatagtcatatcg |
| | | agtgtgcacttactgtatatcttttcat |
| 112 | 204326 | gcgtgttttcctcttgatcgggaactcctgcttctccttgcctcgaaatggaccccaactgctcctgctcgcctgttggctcctgtgcc |
| | _x_at | tgtgccggctcctgcaaatgcaaagagtgcaaatgcacctcctgcaagaagagctgctgctcctgctgccctgtgggctgtgcnaagtg |
| | | tgcccagggctgcatctgcaaagggacgtcagacaagtgcagctgctgtgcctgatgccaggacagctgtgctctcagatgtaaataga |
| | | gcaacctatataaacctggattttttttttttttttttgtacaaccctgacccgtttgctacatctttttttctat |
| 113 | 204388 | ttccacatcagtaactgccctggggtttgtgctgtacaaatacaagctcctgccacggtcttgaagttctgttcttatgctctctgctc |
| | _s_at | actggttttcaataccaccaagaggaaaatattgacaagtttaaaggctgtgtcattgggccatgtttaagtgtactggatttaactac |
| | | ctttggcttaattccaatcattgttaaagtaaaaacaattcaaagaatcacctaattaatttcagtaagatcaagctccatcttatttg |
| | | tcagtgtagatcaactcatgttaattgatagaataaagccttgtgatcact |
| 114 | 204389 | ctttgcgggcacagagactgccacaaagtggagcggctacatggaaggggcagttgaggctggagaacgagcagctagggaggtcttaa |
| | _at | atggtctcgggaaggtgaccgagaaagacatctgggtacaagaacctgaatcaaaggacgttccagcggtagaaatcacccacaccttc |
| | | tgggaaaggaacctgccctctgtttctggcctgctgaagatcattggattttccacatcagtaactgccctggggtttgtgctgtacaa |
| | | atacaagctcctgccacggtcttgaagttctgttcttatgctctctgctcactggttttcaataccaccaagaggaaaatattgacaag |
| | | tttaaaggctgtgtcattgggccatgtttaagtgtactggatttaactacctttg |
| 115 | 204438 | taattgcttgttttctagcctggcaagatattttcataaaagagggataacaatgctgattactaccttttaaaatattttagataaat |
| | _at | gcacagcaccacagcaccacatctaagcattagtgatgggtagctgatgtcagcttcatgtggattttaagcactctagaaacaatgaa |
| | | gcttcttggcatattttaaggagctcccaaaatgtgttacctattaaattgtaactcagcaagtagaagaccatttgaaaagtcaggta |
| | | caaatttcctcaagtggcataaaaatgtagtcagttttctcttttaccagtttttatttccactccaattatttagaactttatttgta |
| | | catgtgcagaagaataaggcagctgagaatcttgtttcccccaagagagttttacaggctgagtgttgcaaatgtgttctttgtcctgt |
| | | tatatgtat |
| 116 | 204457 | gcactgccatgtatgaaagtctctttatgatgtttgtttttttgtcatttttgttctttacatcaagaaattttatgtttaaatatgcg |
| | _s_at | gagaatgtatattgcctctgctcctatcagggttgctaaaccctggtacatcgtatataaaatgtattaaaactggggtttgttaccag |
| | | ttgctgtactttgtatatagaatttttataaattgtatgcttcagaaataatttatttttaaaaagaaattaaaagattaaactcacat |
| | | ccatattacacctttcccccctgaaatgtatagaatccatttgtcatcaggaatcaaaacccacagtccattgtgaagtgtgctatatt |
| | | tagaacagtcttaaaatgtacagtgtattttatagaattgaagttaacattcttattttcaagagaatttatggacgttgtagaaatgt |
| | | acaaatgcatttccaaactgccttaaacgttgtatt |
| 117 | 204508 | gggactgtctgtcaaaagactctgtatatcttttgtggatgagttttgtgagagaacagagagaccattgtacctggcacaagggctct |
| | _s_at | tcatgaaaagggagacttactgggaggtgcaagacagtggcatttctcctctcctcttgctgctcagcacagccctggattgcagcccc |
| | | gaggctgagaccagacaaagcccgggaggcagaaagatgctccaagaaccaacactatcaatgtctttgcaaatcctcacaggattcct |
| | | gtgggtccagctttggaactgggaaacctttcttcggatccgcactcattccactgatgccagctgcccctgaaggatgccagtactgt |
| | | ggtgtgtgagtctcagcagccgcccacacgctcctaactctgctgcatggcagatgcctaggtgga |
| 118 | 204532 | attaatcagccccagagtgctttaaaaaattctcttaaataaaaataatagactcgctagtcagtaaagatatttgaatatgtatcgtg |
| | _x_at | ccccctccggtgtctttgatcaggatgacatgtgccatttttcagaggacgtgcagacaggctggcattctagattacttttcttactc |
| | | tgaaacatggcctgtttgggagtgcgggattcaaaggtggtcccaccgctgcccctactgcaaatggcagttttaatcttatcttttgg |
| | | cttctgcagatggttgcaattgatccttaaccaataatggtcagtcctcatctctgtcctgcttcataggtgccaccttgtgtgtttaa |
| | | a |
| 119 | 204570 | ctacgcgtgtccttgggcggagaagggaggtgactccggcggaagaggacaaggcagaatgcaggcccttcgggtgtcccaggcgctga |
| | _at | tccgctccttcagctccaccgcccggaaccgctttcagaaccgagtgcgcgagaaacagaagctcttccaggaggacaatgacatcccg |
| | | ttgtacctgaagggcggcatcgttgacaacatcctgtaccgagtgacaatgacgctgtgtctgggcggcactgtctacagcttgtactc |
| | | ccttggctgggcctccttccccaggaattaagaccaagaagcctggggggcctgagagact |
| 120 | 204607 | tggtgacacaaacagccttttcccaggtacttggtacctggagcgagtggacgagcagcatcgccgaaagtatgcccggcgtcccgtct |
| | _at | aaaggtgttctgcagatccatggaaagcttcctgggaaacgtatgctagcagagcttctccccgtgaatcatatttttaagatcccact |
| | | cttagctggtaaatgaatttgaatcgacatagtagccccataagcatcagccctgtagagtgaggagccatctctagcgggcccttcat |
| | | tcctctccatgctgcaatcactgtcctgggcttatggtgcctatggactaggggtcctttgtgaaagagcaagatggagcaatggagag |
| | | aagacctcttcctgaatcactggactccagaaatgtgcatgcagatcagctgttgccttca |
| 121 | 204673 | ttgatgccagcatttgcatcccgggctccatcacattcatgcccaatggatgctgcaagacctgcaccgttctcgcaatgagaccaggg |
| | _at | tgccctgctccaccgtccccgtcaccacggaggtttcgtacgccggctgcaccaagaccgtcctcatgaatcattgctccgggtcctgc |
| | | gggacatttgtcatgtactcggccaaggcccaggccctggaccacagctgctcctgctgcaaagaggagaaaaccagccagcgtgaggt |
| | | ggtcctgagctgccccaatggcggctcgctgacacacacctacacccacatcgagagctgccagtgccaggacaccgtctgcgggctcc |
| | | ccaccggcacctcccgccgggcccggcgctcccctaggcatctggggagcgggtgagcggggtgggcacagcccccttcactgccctcg |
| | | acagctttacctcccccggaccctctgagcctcctaagctcggcttcctctcttcagatatttattgtctgagtctttgttcagtcctt |
| | | g |
| 122 | 204688 | cttgcttatatcatgtgctgccgacgggaaggcgtggaaaagagaaacatgcaaacaccagacatccaactggtccatcacagtgctat |
| | _at | tcagaaatctaccaaggagcttcgagacatgtccaagaatagagagatagcatggcccctgtcaacgcttcctgtgttccaccctgtga |
| | | ctggggaaatcatacctcctttacacacagacaactatgatagcacaaacatgccattgatgcaaacgcagcagaacttgccacatcag |
| | | actcagattccccaacagcagactacaggtaaatggtatccctgaagaaagaaaactgactgaagcaatgaatttataatcagacaata |
| | | tagcagttacatcacatttcttttctcttccaataatgcatgagcttttctggcatatgtt |
| 123 | 204697 | cctcccgggaggacagccttgaggcgggcctgcccctccaggtccgaggctaccccgaggagaagaaagaggaggagggcagcgcaaac |
| | _s_at | cgcagaccagaggaccaggagctggagagcctgtcggccattgaagcagagctggagaaagtggcccaccagctgcaggcactacggcg |
| | | gggctgagacaccggctggcagggctggccccagggcaccctgtggccctggctctgctgtccccttggcaggtcctggccagatggcc |
| | | cggacgctgcttccggtagggaggcagcctccagcctgcccaagcccaggccaccctatcgccccctacgcgccttgtctcctactcct |
| | | gactcctacctgccctggaacatcctttgcagggcagccccacaactttaaacattgacgattccttctctgaacacaggcagctttct |
| | | agaagtttcccttcctccatcctatccactgggcacaactgcaataacttctgaccttttggtgaaagctgagaactcctgactgtaac |
| | | atattctg |
| 124 | 204719 | gcaggccatactggttccattgttctgtataatactgaataaataaatttacttttacatgatcgtataagtttctagataagataaac |
| | _at | aaattctgtttaaatttttttaataaaaatcttaaaacactttttctaacctagactgagaaattcatgtttacttttctaggtgtatg |
| | | atactttgtaaagttgatactttcctaagaatttaacatgtcatatttttgaaatagatttaagtgtgcttcttattgctaaaaatact |
| | | aaatgtcatgggtcatagtatctgatatcaatatcgttgataacatatccacaggtaacaccatgatgtaggcataaatggaaaacaaa |
| | | aaccctactatttcaaatatattgtacttttttatttctgtaagccaactgtgtgccattttcactggacttttaaatctagactttag |
| | | tgatgtctacattgtaaatgatcttttgtggatatttgtcacttggtttcagaaagttcacaaatgtagcaacagctcacatgactgag |
| | | t |
| 125 | 204745 | tcccttctcgcttgggaactctagtctcgcctcgggttgcaatggaccccaactgctcctgtgccgctggtgtctcctgcacctgcgcc |
| | _x_at | agctcctgcaagtgcaaagagtgcaaatgcacctcctgcaagaagagctgctgctcctgctgccctgtgggctgtgccaagtgtgccca |
| | | aggctgcatctgcaaaggggcatcggagaagtgcagctgctgcgcctgatgtcgggacagccctgctcccaagtacaaatagagtgacc |
| | | cgtaaaatctaggattttttgttttttgctacaatcttgacccct |
| 126 | 204818 | aaaggctggcatcttatggctcatcaaaggcggctgtgaccatgttctcatcagttatgagactggagctttccaagtggggaattaaa |
| | _at | gttgcttccatccaacctggaggcttcctaacaaatatcgcaggcaccagtgacaagtgggaaaagctggagaaggacattctggacca |
| | | cctccccgctgaggtacaggaagactacggccaggactacatcttagcacagcggaatttcctcctattgatcaactcgttagccagca |
| | | aggacttctctccggtgctgcgggacatccagcatgctatcttggcgaagagcccttttgcctattacacgccagggaaaggcgcttac |
| | | ttgtggatctgccttgctcactatttgcctattggca |
| 127 | 204834 | ggtgtcgaactatatgccttgtatgatcagttttatgtggctaatgagtttctcaaatatcgtttacacgttggtaactataatggcac |
| | _at | agctggagatgcattacgtttcaacaaacattacaaccacgatctgaagtttttcaccactccagataaagacaatgatcgatatcctt |
| | | ctgggaactgtgggctgtactacagttcaggctggtggtttgatgcatgtctttctgcaaacttaaatggcaaatattatcaccaaaaa |
| | | tacagaggtgtccgtaatgggattttctggggtacctggcctggtgtaagtgaggcacaccctggtggctacaagtcctccttcaaaga |
| | | ggctaagatgatgatcagacccaagcactttaagccataaatcactctgttcattcctccaggtattcgttatctaatagggcaattaa |
| | | ttccttcagcacttt |
| 128 | 204894 | ccaggaggggctgtccaatcacattcaggcatgcgaatgagctgggccctgggtgaggtgggggtctggcctagtggggaggggcctgg |
| | _s_at | cctgggtggggcagggcctggcctggtccaggcttgggctccattcccatcactgctgtccctcctgaggtctggattggggatgggga |
| | | caaagaaatagcaagagatgagaaacaacagaaacttttttctctaaaggactggttaaatcaattctgatacagccttacaatacaat |
| | | agtatgcagctaaaaaataattgtatgtctttatatactaatatgtaataatcttcaggtgaaaaaggcaagccacagaaatgtgtata |
| | | gcgcacttcccatttgtgtttcagaaaggagtagaatataaacacataattgcttatgtatgcctattcagaataaatgggtaacactg |
| | | attacttttgggaggggaaccagtaggttgaggacaggagagggaagggtcttaacacttacacccttttgtacattttg |
| 129 | 204895 | aggagcccaggaacgacgtggtcttccagcccatttccggggaagacgtgcgcgatgtgacagccctgaacgtgagcacgctgaaggct |
| | _x_at | tacttcagatgcgatggctacaagggctacgacctggtctacagcccccagagcggcttcacctgcgtgtccccgtgcagtaggggcta |
| | | ctgtgaccatggaggccagtgccagcacctgcccagtgggccccgctgcagctgtgtgtccttctccatctacacggcctggggcgagc |
| | | actgtgagcacctgagcatgaaactcgacgcgttcttcggcatcttctttggggccctgggcggcctcttgctgctgggggtcgggacg |
| | | ttcgtggtcctgcgcttctggggttgctccggggccaggttctcctatttcctgaactcagctgaggccttgccttgaaggggcagctg |
| | | tggcctaggctacctcaagactcacctcatccttaccgcacatttaaggcgccattgcttttgggagact |
| 130 | 204897 | agcagcttattgtttctctgaaagtgtgtgtagttttactttcctaaggaattaccaagaatatcctttaaaatttaaaaggatggcaa |
| | _at | gttgcatcagaaagctttattttgagatgtaaaaagattcccaaacgtggttacattagccattcatgtatgtcagaagtgcagaattg |
| | | gggcacttaatggtcaccttgtaacagttttgtgtaactcccagtgatgctgtacacatatttgaagggtctttctcaaagaaatatta |
| | | agcatgttttgttgctcagtgattgtgaattgcttggttgtaattaaattctgagcctgatattgatatg |
| 131 | 204931 | gagaacgggtacattcacccggtcaacctgacgtggccctttatggtggccgggaaacccgagagtgacctgaaagaagtggtgaccgc |
| | _at | gagccgcttatgtggaaccaccgcgtcctgaccttggaggtgcgagtctgggaaaggcgcgctcccggggggagcgggccccgggaagg |
| | | cgacccctgccctcagtgctctctgtctctgcttccccctcgcaatgctcctctctctgtcccaccccgcgagaacactttacaacgac |
| | | gaggagattcgtttccaaaccagaggagatcaattgtacttacaaagattcccatctatttaactttattaacttctaccgtgaatgac |
| | | tctgcaagccttgctggtcca |
| 132 | 204938 | ttctctcgaccacttaaaacttcagacttcctgtcctgctggtatcatggagaaagtccaatacctcactcgctcagctataagaagag |
| | _s_at | cctcaaccattgaaatgcctcaacaagcacgtcaaaagctacagaatctatttatcaatttctgtctcatcttaatatgtctcttgctg |
| | | atctgtatcatcgtgatgcttctctgaagttctgctacaacctctagatctgcagcttgccacatcagcttaaa |
| 133 | 204939 | tggcccatctattacatctacagctgacccttgaacatgggggttaggggagctgacaattcgtgggtccgcaaaatcttaactaccta |
| | _s_at | atagcctactattgaccataaaccttactgataacataaacagtaaattaacacatattttgcgtgttatatgtattatacactatatt |
| | | cctacaataaagt |
| 134 | 204940 | aaatctgttctaagacatatgatcaacagatgagaactggtggttaatatgtgacagtgagattagtcatatcactaatatactaacaa |
| | _at | cagaatctaatcttcatttaaggcactgtagtgaattatctgagctagagttacctagcttaccatactatatctttggaatcatgaaa |
| | | ccttaagacttcagaatgattttgcaggttgtcttccattccagcctaacatccaatgcaggcaaggaaaataaaagatttccagtgac |
| | | agaaaaatatattatctcaagtattttttaaaaatatatgaattctctctccaaatattaactaattattagattatattttgaaatga |
| | | acttgttggcccatc |
| 135 | 204955 | ggacaaagagcgctatgtctccctggtgatgcctgtggccctgttcaacctgattgacacttttcccttgagaaaagaagagatggtcc |
| | _at | tacaagccgaaatgagccagacctgtaacacctgacatgatggttcctctcttggcaattcctcttcattgtctacatagtgacatgca |
| | | cacgggaaagccttaaaaatatccttgatgtacagattttatttgtaattttaaaagtctattttattatgagctttctttgcacttaa |
| | | aaattagcatgctgctttttgtacttggaagtgtttcaaaaaattatatgaccatatttactctttctaacctttctttactccatcat |
| | | ggctggttgatttgtagagaaattagaacccataaccatacacaggctatcaacatgttattcaatgtgacacctaactcttttc |
| 136 | 205097 | tactcatgcctttttgtttaggataaataggtaagcacaaagagctcttcaaaatcagaaaaaacaataggagtccttccttgtctttt |
| | _at | ctgtgatctctgtccttgtttctgagactttctctaccattaagctctattttagctttcagttattctagtttgtttcccatggaatc |
| | | tgtcctaaactggtgtttttgtcagtgacagtcttgccagtcagcaatttctaacagcattttaaatgagtttgatgtacagtaaatat |
| | | tgatgacaatgacagcttttaactcttcaagtcacctaaagctattatgcaggaggatttagaagtcacattcataaaacccaagngct |
| | | atgggtgtattattcatgatagctggcccacaggtcatgaattgag |
| 137 | 205112 | agaagctcaccaagtcaactaaacagccccgaggacttacatcaccttctcagctcttgacctcagaaagtatccaaaccaaggaggag |
| | _at | aaacctgtgggtggcttgtcctccagtgacacaatggattaccgacagtgactaagggcagcatgtttaacccaggtgaagatct |
| 138 | 205200 | tttctggccttcacccagacgaagaccttccacgaggccagcgaggactgcatctcgcgcgggggcaccctgagcacccctcagactgg |
| | _at | ctcggagaacgacgccctgtatgagtacctgcgccagagcgtgggcaacgaggccgagatctggctgggcctcaacgacatggcggccg |
| | | agggcacctgggtggacatgaccggcgcccgcatcgcctacaagaactgggagactgagatcaccgcgcaacccgatggcggcaagacc |
| | | gagaactgcgcggtcctgtcaggcgcggccaacggcaagtggttcgacaagcgctgccgcgatcagctgccctacatctgccagttcgg |
| | | gatcgtgtagccggc |
| 139 | 205259 | gcagactgggagttgctagcaaacaaatggcttacttacaaaagcagcttttagttcagacttagtttttataaaatgagaattctgac |
| | _at | ttacttaaccaggtttgggatggagatggtctgcatcagctttttgtattaacaaagttactggctctttgtgtgtctccaggtaactt |
| | | tgcttgattaaacagcaaagccatattctaaattcactgttgaatgcctgtcccagtccaaattgtctgtctgctcttatttttgtacc |
| | | atattgctcttaaaaatcttggtttggtacagttcataattcaccaaaaagttcatataatttaaagaaacactaaattagtttaaaat |
| | | gaagcaatttatatctttatgcaaaaacatatgtctgtctttgcaaagga |
| 140 | 205267 | gcacccagcatttaccatgtgggtttctttagtgtcttaaaagcgtccataagccaccattctgtggaaccaaggccccctccacgcaa |
| | _at | acaccctccctcctggggacctctggagcctcagccagaagtaccattaggtttaattttaatttgttttgctggagaaacatcaggtt |
| | | tgtaggagactgagttgttagcaggtgtgcttagctcttgatagtgaacgtgtaccttgggaactggctcacccacctgctaatagcac |
| | | catcgtcactattaagcagacatttcagttggtagaatccatgtagaagtcatggacttttctgggaaatgacttttctgggaaatgac |
| | | agtttctttgacatattttctttgcccactt |
| 141 | 205382 | cgccatcaccgagcgcttgatgtgcgcggagagcaatcgccgggacagctgcaagggtgactccgggggcccgctggtgtgcgggggcg |
| | _s_at | tgctcgagggcgtggtcacctcgggctcgcgcgtttgcggcaaccgcaagaagcccgggatctacacccgcgtggcgagctatgcggcc |
| | | tggatcgacagcgtcctggcctagggtgccggggcctgaaggtcagggtcacccaagcaacaaagtcccgagcaatgaagtcatccact |
| | | cctgcatctggttggtctttattgagcacctactatatgcagaaggggag |
| 142 | 205403 | gggccacgccaggaatattcagaaaataatgagaactacattgaagtgccattgatttttgatcctgtcacaagagaggatttgcacat |
| | _at | ggattttaaatgtgttgtccataataccctgagttttcagacactacgcaccacagtcaaggaagcctcctccacgttctcctggggca |
| | | ttgtgctggccccactttcactggccttcttggttttggggggaatatggatgcacagacggtgcaaacacagaactggaaaagcagat |
| | | ggtctgactgtgctatggcctcatcatcaagactacaatcctatcccaa |
| 143 | 205412 | gctgctctggttctcatgacggcagatgcagcgaagaggctcaatgttacaccactggcaagaatagtagcatttgctgacgctgctgt |
| | _at | agaacctattgattttccaattgctcctgtatatgctgcatctatggttcttaaagatgtgggattgaaaaaagaagatattgcaatgt |
| | | gggaagtaaatgaagcctttagtctggttgtactagcaaacattaaaatgttggagattgatccccaaaaagtgaatatcaatggagga |
| | | gctgtttctctgggacatccaattgggatgtctggagccaggattgttggtcatttgactcatgccttgaagcaaggagaatacggtct |
| | | tgccagtatttgcaatggaggaggaggtgcttctgccatgctaattcagaagctgtagacaacctctgctatttaaggagacaacccta |
| | | tgtgaccagaa |
| 144 | 205433 | ggaaagcaggattccatcgctggaacaattacatgatggactggaaaaatcaatttaacgattacactagcaagaaagaaagttgtgtg |
| | _at | ggtctctaattaatagatttaccctttatagaacatattttcctttagatcaaggcaaaaatatcaggagcttttacacacctactaaa |
| | | aaagttattatgtagctgaaacaaaaatgccagaaggataatattgattcctcacatctttaacttagtattttacctagcatttcaaa |
| | | acccaaatggctagaacatgtttaattaaatttcacaatataaagttctacagttaattatgtgcatattaaaacaatggcctggttca |
| | | atttctttctttccttaataaatttaagttttttccccccaaaattatcagtgctctgcttttagtcacgtgtattttcattaccactc |
| | | gtaaaaaggtatcttttttaaatgaattaaatattgaaacactgtacaccatagtttaca |
| 145 | 205464 | gagggtgatgccatctaaccctgcccctgtccaccccgggtgggtgaaactcactgagcagccaagactgttgcccgaggactcactgt |
| | _at | atggtgccctctccaaagggtcgggagggtagctctccaggccagagcttgtgtccttcaacagagaggccagcggcaactggtccgtt |
| | | actggccaagggctctgaagaatcaacggtgctggtacaggatacaggaataaattgtatcttcacctggttcctaccctcgtccctac |
| | | ctgtcctgatcctggtcctgaagacccctcggaacaccctctcctggtggcaggccacttccctcccagtgccagtctccatccacccc |
| | | agagaggaacaggcgggtgggccatgtggttttctccttcctggccttggctggcctctggggcaggggtggtggagagatggaagggc |
| | | atcaggtgtagggaccctgccaagtggcacctgatttactctag |
| 146 | 205480 | ttaatgtgccaaggagccgttttctgcctgtcaaaaccacatcagatctcttgctggtgatgtcaaacctctatagtcttaatgcagga |
| | _s_at | tctctgacaatgagtgaaaagcgggaatttcctacagtgcccttggttaaattaggcagttcttttacgaaggttcaagattatctaag |
| | | aagatttgaaagtataccagatatgcttgaattggatcacctcacagtttcaggagatgtgacatttggaaaaaatgtttcattaaagg |
| | | gaacggttatcatcattgcaaatcatggtgacagaattgatatcccacctggagcagtattagagaacaagatagtgtctggaaacctt |
| | | cgcatcttggaccactgaaatgaaaaatactgtggacacttaaataatgggctagtttcttacaatgaaatgttctctaggatttaggc |
| | | actaaaaggtactttactatgttactgtaccctgc |
| 147 | 205547 | caagaatgatgggcactaccgtggagatcccaactggtttatgaagaaagcgcaggagcataagagggaattcacagagagccagctgc |
| | _s_at | aggagggaaagcatgtcattggccttcagatgggcagcaacagaggggcctcccaggccggcatgacaggctacggacgacctcggcag |
| | | atcatcagttagagcggagagggctagccctgagcccggcgctcccccagctccttggctgcagccatcccgcttagcctgcctcaccc |
| | | acacccgtgtggtaccttcagccctggccaagctttgaggctctgtcactgagcaatggtaactgcacctgggcagctcctccctgtgc |
| | | ccccagcctcagcccaacttcttacccgaaagcatcactgccttggcccctccctcccggcggcccccatcacctctactgtctcctcc |
| | | ctgggctaagcaggggagaagcgggctgggggtagcctggatgtgggcgaagtccactgtcctccttggcggcaaaagcccatt |
| 148 | 205554 | tcaaagacttcgtgattatccccctgcacaccaccccagagacatccgttaaggagatcgatgagttggttgaggtctacacggacgtg |
| | _s_at | aaacaccgctggaaggcggagaatttcattttcatgggtgacttcaatgccggctgcagctacgtccccaagaaggcctggaagaacat |
| | | ccgcttgaggactgaccccaggtttgtttggctgatcggggaccaagaggacaccacggtgaagaagagcaccaactgtgcatatgaca |
| | | ggattgtgcttagaggacaagaaatcgtcagttctgttgttcccaagtcaaacagtgtttttgacttccagaaagcttacaagctgacg |
| | | aagaggaggccctggatgtcagcgaccactttccagttgaatttaaactacagtcttcaagggctcttcaccaacagcaaaaaatctgt |
| | | cactctaaggaagaaaacaaagagcaaacgctcctagacccaagggtctcatcttattaaccatttcttgcctctaaata |
| 149 | 205593 | aacgaggagcacatgaccctgctgaagatgattttgataaaatgctgtgatatctctaacgaggtccgtccaatggaagtcgcagagcc |
| | _s_at | ttgggtggactgtttattagaggaatattttatgcagagcgaccgtgagaagtcagaaggccttcctgtggcaccgttcatggaccgag |
| | | acaaagtgaccaaggccacagcccagattgggttcatcaagtttgtcctgatcccaatgtttgaaacagtgaccaagctcttccccatg |
| | | gttgaggagatcatgctgcagccactttgggaatcccgagatcgctacgaggagctgaagcggatagatgacgccatgaaagagttaca |
| | | gaagaagactgacagcttgacgtctggggccaccgagaagtccagagagagaagcagagatgtgaaaaacagtgaaggagactgtgcct |
| | | gaggaaagcggggggcgtggctgcagttctggacgggctggccgagctgcgcgggatccttgtgcagggaagagctgccctgggcacct |
| | | ggcaccacaagaccatgttttctaa |
| 150 | 205683 | tgacgcaaaataccaccttggcgcctacacgggagacgacgtccgcatcgtccgtgacgacatgctgtgtgccgggaacacccggaggg |
| | _x_at | actcatgccagggcgactccggagggcccctggtgtgcaaggtgaatggcacctggctgcaggcgggcgtggtcagctggggcgagggc |
| | | tgtgcccagcccaaccggcctggcatctacacccgtgtcacctactacttggactggatccaccactatgtccccaaaaagccgtgagt |
| | | caggcctgggttggccacctgggtcactggaggaccaacccctgctgtccaaaacaccactgcttcctacccaggtggcgactgccccc |
| | | cacaccttccctgccccgtcctgagtgccccttcctgtcctaagccccctgctctcttctgagccccttcccctgtcctgaggaccctt |
| | | ccctatcctgagcccccttccctgtcctaagcctgacgcctgcaccgggccctccagccctcccctgcccagatagctggtggtgggcg |
| | | ctaatcct |
| 151 | 205892 | gaagagggagctctattgccaccatgagtttctccggcaagtaccaactgcagagccaggaaaactttgaagccttcatgaaggcaatc |
| | _s_at | ggtctgccggaagagctcatccagaaggggaaggatatcaagggggtgtcggaaatcgtgcagaatgggaagcacttcaagttcaccat |
| | | caccgctgggtccaaagtgatccaaaacgaattcacggtggg |
| 152 | 205929 | ccttctctggtctccttgagatgatcgtagacacagggatgattcccacccaaacccacgtattcattcagtgagttaaacacgaattg |
| | _at | atttaaagtgaacacacacaagggagcttgcttgcagatggtctgagttcttgtgtcctggtaattcctctccaggccagaataattgg |
| | | catgtctcctcaacccacatggggttcctggttgttcctgcatcccgatacctcagccctggccctgcccagcccatttgggctctggt |
| | | tttctggtggggctgtcctgctgccctcccacagcctccttctgtttgtcgagcatttcttctactcttgagagctcaggcagcgttag |
| | | ggctgcttaggtctcatggaccagtggctggtctcacccaactgcagtttactattgctatcttttctggatgatcagaaaaataattc |
| | | cataaatctattgtctacttgcgattttttaaaaaatgtatatttttatatatattgttaaatccttgcttcattcca |
| 153 | 205935 | tctcccaacctctactgtaaactttctggtccgagaacgagccgaacacagcgcgacgcagggactaggacggcccggtgaccgcgcgg |
| | _at | attcaggattgcggggacgcagaaaggttaaggcacttttaaaaactatagcaaggctcctgtttatttattctactttctttccctaa |
| | | taatcaaaacaccgcgtaggctcctccgtttatcagtattaatggtgtaactttgttggcaatatttgccgtgtagaattttttttaga |
| | | tatccattgtaaatttgaaacaaagaccgatctgtgtaaaaacaaatttccatatgttttatataaatatatatataatatgaaggact |
| | | accctcctttttttttttttgtattttggctgctagagtgcagcatttgtgacacgtatttgaaatttgaaatttccttctgcactgta |
| | | taaaaggaccatttgaggatgttttgccttttgtgtatttt |
| 154 | 205950 | gagccccattcacaaattttgacccctctactctccttccttcatccctggatttctggacctaccctggctctctgactcatcctcct |
| | _s_at | ctttatgagagtgtaacttggatcatctgtaaggagagcatcagtgtcagctcagagcagctggcacaattccgcagccttctatcaaa |
| | | tgttgaaggtgataacgctgtccccatgcagcacaacaaccgcccaacccaacctctgaagggcagaacagtgagagcttcattttgat |
| | | gattctgagaagaaacttgtccttcctcaagaacacagccctgcttctgacataatccagttaaaataataatttttaagaaataaatt |
| | | tatttcaatattagcaagacagcatgccttcaaatcaatctgtaaaactaagaaacttaaattttagttcttactgcttaattcaaata |
| | | ataattagtaagctagcaaatagtaatctgtaagcataagcttatcttaaattcaagtttagtttgaggaattctttaaaattacaact |
| | | aagtgatttgtatgtctatttttttc |
| 155 | 206000 | ggcagcttttttatcagccttgctttggataggacctccaaggactaagcctccagccccatgtgtgacccttgtcatctctctgcccc |
| | _at | acataattatgttactttgctatgtgctcctaatgtatctagtgtgtcctgtgacaacactcatcacacttcattgtaaatcacttgtt |
| | | ttattgactgtctttcctatagactgtaagctccatgagggcaggcacatgttgttctcattgaccgtgctggccccagtgcctagatg |
| | | catggctggcacattgttggcactcaacaatggttgaatgaataaaacaataaatgaatgaataactaagatatagaaactctcattta |
| | | tattgcagattgaatatatatgatgaaattcttatgttgaatatgttagaatcaaatactcatttttcattagatacagtagtgtcatc |
| | | actcttttaagatcttgttaaagatttcaaataaaggtacttctggcgagccaggctgcacagcatttgctttcct |
| 156 | 206094 | tatttgaatatgtatcgtgccccctccggagtctttgatcaggatgacatgtgccatttttcagaggacgtgcagacaggctggcattc |
| | _x_at | tagattacttttcttactctgaaacatggcctgtttgggagtgcgggattcaaaggtggtcccacggctgcccctactgcaaatggcag |
| | | ttttaatcttatcttttggcttctgcagatggttgcattgatccttaaccaataatggtcagtcctcatctctgtcctgacttcatagg |
| | | t |
| 157 | 206134 | ctttctatattgttatcagtccaggaaacaggtaaacagatgtaattagagacattggctctttgataggcctaatctttctttttact |
| | _at | tttttttttcttttttctttttttttaaagatcatgaatttgtgacttagttctgccctttggagaacaaaagaaagcagtcttccatc |
| | | aaatcaccttaaaatgcacggctaaactattcagagttaacactccagaattgttaaattacaagtactatgctttaatgcttctttca |
| | | tcttactagtatggcctataaaaaaaataataccacttgatgggtgaaggctttggcaatagaaagaagaatagaattcaggttttatg |
| | | ttattcctctgtgttcacttcgccttgctcttgaaagtgcagtatttttctacatcatgtcgagaatgattcaatgtaaatatttttca |
| | | ttttatcatgtatatcctatacacacatctccttcatcatcatatataagtttattttgagaagtctacattgcttacattt |
| 158 | 206143 | taacaatccatgatgctgttttgcatattgatgaagaaagattacagtacttcaaagtttaatcccagtcaggaaaaagatggaaaaat |
| | _at | tgattttaccataaatacaaatggaggattacgtaatcgggtatatgaggtgccagttgaaacaaaattctaatcaacatataattcag |
| | | aaggatcttcatctgactatgacataaaaacaactttatacccagaaagttattgataagttcatacattgtacgaagagtatttttga |
| | | cagaatatgtttcaaactttggaacaagatggttctagcatggcatatttttcacatatctagtatgaaattatataagtattctaaat |
| | | tttatatcttgtagctttatcaaagggtgaaaattattttgttcatacatatttttgtagcactgacagatttccatcctagtcactac |
| | | cttcatgcataggtttagcagtatagtggcgccactgttttgaatct |
| 159 | 206149 | tcatcaggctccagttattctccatctcccagctcagctttttctgtctgtaagcctgattttcaggaaggctctttcctagtgatgga |
| | _at | gatgaccaccatcagctccaggcttctatcctgctaacccagtaacccagtgggaagagatttacttattccaataattccaagtggag |
| | | agtgtcattgacccgtttggggtctcatctctacttctaggggaatgaaacactttgagtggccaggcctgtgtcatgtgctaattcct |
| | | agagccagggaaataaggtctgaggattcaggatggggtgaaaggtggttgcttaaaggaaaatgaaatacaattagcagaataagggg |
| | | aaacgagtggtctgctctgctcgggcaaaacaagagatgcccattactgtgagggacccttgaagtctggactcttaaatgggtttttg |
| | | ctgatttcctgggtgcatgctagg |
| 160 | 206198 | accagcaacaacttcaatccggtggagaacaaagatattgtggttttaacctgtcaacctgagactcagaacacaacctacctgtggtg |
| | _s_at | ggtaaacaatcagagcctcctggtcagtcccaggctgctgctctccactgacaacaggaccctcgttctactcagcgccacaaagaatg |
| | | acataggaccctatgaatgtgaaatacagaacccagtgggtgccagccgcagtgacccagtcaccctgaatgtccgctatgagtcagta |
| | | ca |
| 161 | 206199 | atattagttaccctggtgtgctgtattctctaaaacctttaaatgtttgcatgcagccattcgtcaaatgtcaaatattctctctttgg |
| | _at | ctggaatgacaaaaactcaaataaatgtatgattaggaggacatcataacctatgaatgatggaagtccaaaatgatggtaactgacag |
| | | tagtgttaatgccttatgtttagtcaaactctcatttaggtgacagcctggtgactccagaatggagccagtcatgctaaatgccatat |
| | | actcacactgaaacatgaggaagcaggtagatcccagaacagacaaaactttcctaaaaacatgagagtccaggctgtctgagtcagca |
| | | cagtaagaaagtcctttctgctttaactcttagaaaaaagtaatatgaagtattctgaaattaaccaatcagtttatttaaatcaattt |
| | | atttatattcttctgttcctggattcccattttacaaaacccactgttctactgttgtattgcccagt |
| 162 | 206208 | cccaggaccctgaagacgaaattgcggtgctggcctttctggtggaggctggaacccaggtgaacgagggcttccagccactggtggag |
| | _at | gcactgtctaatatccccaaacctgagatgagcactacgatggcagagagcagcctgttggacctgctccccaaggaggagaaactgag |
| | | gcactacttccgctacctgggctcactcaccacaccgacctgcgatgagaaggtcgtctggactgtgttc |
| | | cgggagcccattcagcttcacagagaacagatcctggcattctctcagaagctgtactacgac |
| | | aaggaacagacagtgagcatgaaggacaatgtcaggcccctgca |
| 163 | 206209 | accacaccgacctgcgatgagaaggtcgtctggactgtgttccgggagcccattcagcttcacagagaacagatcctggcattctctca |
| | _s_at | gaagctgtactacgacaaggaacagacagtgagcatgaaggacaatg |
| | | tcaggcccctgcagcagctggggcagcgcacggtgataaagtccggggccccgggtcggccgctgccc |
| | | tgggccctgcctgccctgctgggccccatgctggcctgcctgctggccggcttcctgcgatgatggctcacttctgcac |
| 164 | 206262 | actgactggacgcacgtggaaaggagctatttttggaggctttaagagtaaagaatctgtcccgaaacttgtggctgactttatggcta |
| | _at | agaagttttcactggatgcattaataacaaatattttaccttttgaaaaaataaatgaaggatttgacc |
| | | tgcttcgctctggaaagagtatccgtaccgtcctgacgttttgaaacaatacagatgcct |
| | | tcccttgtagcagttttcagcctcctctaccctacatgatctggagcaacagctaggaaatatcattaattctgctcttcagagatgt |
| | | taaaaataaattacacgtgggagctttccaa |
| 165 | 206377 | gttggtcactgttatttgcctactgctggaagaaggacaaccgctggcaaggtagcgttccccaatctgaatacctgcaggctccca |
| | _at | catgagggagagggcagactcaggtgggaagatgtgccatgcgtaaggcatcaacg |
| | | tgtatctgtgggatcttcgttgccttcagtaatcagggtgtgaaaaaagcagacaagttgtgtgtgtg |
| | | tgtgtgtgtctaagaaaacttgtgtgcttttcaaaaaggcagtgctaagcacaagatttcaagaaagcctcttcttgttgcc |
| | | tagctgagtgggagagtcattttccccagacactacatttggatacaggtgccaaagaacattattaagga |
| | | attatttagaaacaatgtgtctagtttaagaaagtggttttcagtattgtgacaatacaacgttttt |
| | | acaaggttgttttctaccaccatattttaaagatatttttatgaccgtgtatactcacactttgctt |
| 166 | 206385 | aaagcccttcatctaatatttgttgctattgccaatttttcaatgaaatgacctaaaaacaacaaaaaaaaataacctatacgg |
| | _s_at | tagttgctttagggggtggggggatgctatctgttagtgcttaaaagggggtaaatgcttg |
| | | ccgctttagaggtggatggtgctcataaaaggccccagtcgggggtatttaaaaaggactgaacagaaa |
| | | tccttagctagtagaatggcagcacgctgtaaaattattactgtattgtgtactggctataagatgtagacacctttcagtaa |
| | | gccaatcatttgtaaccattctagcagtgtcatattaggttaataaggctgctgtgttttaaagggca |
| | | tttttatttgggttttggtgaaattctttaatttgttgattatattcacataaaatcagcattcattgacacatag |
| | | ctctaatgacatatgtatgaaaaaccatacactggatgacctagtcga |
| 167 | 206422 | ctgatggttctttctctgatgagatgaacaccattcttgataatcttgccgccagggactttataaactggttgattcagaccaaa |
| | _at | atcactgacaggaaataactatatcactattcaagatcatcttcacaacatcacctgctagccacgtgggatgtttgaaa |
| | | tgttaagtcctgtaaatttaagaggtgtattctgaggccacattgctttgcatgccaataaataaattttcttttagtgt |
| | | tgtgtagccaaaaattacaaatggaataaagttttatcaaaatattgctaaaatatcagctttaaaatatgaaagtgctagattct |
| | | gttattttcttcttattttggatgaagtaccccaacctgtttacatttagcgataaaattatttttctatgatataatttgtaaat |
| | | gtaaattattccgatctgacatatctgcattataataataggagaatagaagaactggtagccacagtg |
| | | gtgaaattggaaagagaactttcttcctgaaacctttgtcttaaaaatactcagctttcaatgtatcaa |
| 168 | 206461 | gccggctcctgcaagtgcaaaaagtgcaaatgcacctcctgcaagaagagctgctgctcctgttgccccctgggctgtgccaagt |
| | _x_at | gtgcccagggctgcatctgcaaaggggcgtcagagaagtgcagctgctgtgcctgatgtcgggacagcc |
| | | ctgctgtcagatgaaaacagaatgacacgtaaaatccgaggttttttttttctaca |
| | | actccgactcatttgctacattcctttttttctg |
| 169 | 206561 | ttccctgctggaggatcccaagattaaggagattgctgcaaagcacaaaaaaaccgcagcccaggttctgatccgtttccatat |
| | _s_at | ccagaggaatgtgattgtcatccccaagtctgtgacaccagcacgcattgttgagaacattcaggtctttgacttt |
| | _ | aaattgagtgatgaggagatggcaaccatactcagcttcaacagaaactggagggcctgtaacgtgttgcaatcctctcat |
| | | ttggaagactatcccttcaatgcagaatattgaggttgaatctcctggtgagattatacaggagattctctttcttcgctga |
| | | agtgtgactacctccactcatgtcccattttagccaagcttatttaagatcacagtgaacttagtcctgttatagacgagaat |
| | | cgaggtgctgttttagacatttatttctgtatgttcaactaggatcagaatatc |
| | | acagaaaagcatggcttgaataaggaaatgacaattttttccacttatctgatcagaacaaatgtttattaagcatcagaaact |
| | | ctgccaacactga |
| 170 | 206576 | taagtccagtagtagcaaagccccaaatcaaagccagcaagaccacagtcacaggagataaggactctgtgaacctgacctgc |
| | _s_at | tccacaaatgacactggaatctccatccgttggttcttcaaaaaccagagtctcccgtcctcg |
| | | gagaggatgaagctgtcccagggcaacaccaccctcagcataaaccctgtcaagaggga |
| | | ggatgctgggacgtattggtgtgaggtcttcaacccaatcagtaagaaccaaagcgaccccat |
| 171 | 206637 | tgagcctggggttctggtgttagaatatttttaagtaggctttactgagagaaactaaatattggcatacgttatcagcaact |
| | _at | tcccctgttcaatagtatgggaaaaataagatgactgggaaaaagacacacccacaccgtagaacatatattaatctactggcg |
| | | aatgggaaaggagaccattttcttagaaagcaaataaacttgatttttttaaatctaaaatttacattaatgagtgc |
| | | aaaataacacataaaatgaaaattcacacatcacatttttctggaaaacagacggattttacttctggagacatggcat |
| | | acggttactgacttatgagctaccaaaactaaattctttctctgctattaactggctagaagacattcatctatttttcaaatg |
| | | ttctttcaaaacatttttataagtaatgtttgtatctatttcatgctttact |
| 172 | 206641 | atttctttggcagttttcgtgctaatgtttttgctaaggaagataagctctgaaccattaaaggacgagtttaaaaacacagga |
| | _at | tcaggtctcctgggcatggctaacattgacctggaaaagagcaggactggtgatgaaattattcttccgagaggcctcgag |
| | | tacacggtggaagaatgcacctgtgaagactgcatcaagagcaaaccgaaggtcgactctgaccattgctttccac |
| | | tcccagctatggaggaaggcgcaaccattcttgtcaccacgaaaacgaatgactattgcaagagcctgccagctgc |
| | | tttgagtgctacggagatagagaaatcaatttctgctaggtaattaaccatttcgactcgagcagtgccactttaaaaatct |
| | | tttgtcagaatagatgatgtgtcagatctctttaggatgactgtatttttcagttgccgatacagctttttgtcctctaactg |
| 173 | 206664 | gaagacactaccaacatgatattacgtattgatctgaccacacacaatgttactctagaagaaccaatagaaatcaactggtcatgaa |
| | _at | gatcaccatcaattttagttgtcaatgggaaaaaacaccaggatttaagtttcacagcacttacaattttccctcttcacttggttc |
| | | ttgtactctacaaaatatagctttcataacatcgaaaagttattttgtagcgtacatcaatgataatgctaattttattatagtaat |
| | | gtgacttggattcaattttaaggcatatttaacaaaatttgaatagccctatttatccttgttaagtatcagctacaattgtaa |
| | | actagttactaaacatgtatgtaaatagctaagatataatttaaacgtgatttttaaattaaataaaatttttatgtaattatata |
| | | tactatatttttctcaatgtttagcagatttaagatatgtaacaacaattatttgaagatttaattacttcttagtatgtgcattta |
| 174 | 206710 | attgcaatttcttaggtaaccttatatttacaataaatgaagattaccctcaaatgctagaagctgtctaggtccgtccggtgtgtca |
| | _s_at | gatttcctcagattagatgtgccaataaccaagtttattcagtaaacaacttgtacttgtttcatctggttt |
| | | attactctcacccataaacagtaatgactctctgaccctctggaaatatgtaatgcttccaatcttg |
| 175 | 206784 | ttttggacctgcggtggtggccaaccactggaacttccactggatctactggctgggcccactcctggctggcctgcttgttggact |
| | _at | gctcattaggtgcttcattggagatgggaagacccgcctcatcctgaaggctcggtgagcagagctcgtgggattcctgctgct |
| | | ccaggtgtcctcagctcacctgtcccagactgaggacaggggagttcctgcatttcctgccagggcagaggcccagaggagcgacc |
| | | ccctgcttccactgcttgggcctgctttctcagatagactgactgctgaggaggctctaggttcttggaattcctttgtgctca |
| | | tcagagaccccagcctggggaacacgctgcccgcactgcccagagagcagtgcaaacaccacaacacgagcgtgtttcttgaga |
| | | ggaatgtccccgagttggacaaggaggctgtttctgcacatcagctcatttcc |
| 176 | 207003 | gaggggtcaccgtgcaggatggaaatttctccttttctctggagtcagtgaagaagctcaaagacctccaggagccccaggagc |
| | _at | ccagggttgggaaactcaggaactttgcacccatccctggtgaacctgtggttcccatcctctgtagcaacccgaactttccag |
| | | aagaactcaagcctctctgcaaggagcccaatgcccaggagatacttcagaggctggaggaaatcgctgaggacccgggcaca |
| | | tgtgaaatctgtgcctacgctgcctgtaccggatgctaggggggcttgcccactgcctgcctcccctccgcagcagggaag |
| | | ctcttttctcctgcagaaagggccacccatgatactccactcccagcagctcaacctaccctggtccagtcgggaggagcagcc |
| | | cggggaggaactgggtgact |
| 177 | 207080 | tcgtctgcttcacaagctatcgctatggtgttcgtgcgcaggccgtggcccgccttgaccacagtgcttctggccctgctcgt |
| | _s_at | ctgcctaggggcgctggtcgacgcctaccccatcaaacccgaggctcccggcgaagacgcctcgccggaggagctgaaccgc |
| | | tactacgcctccctgcgccactacctcaacctggtcacccggcagcggtatgggaaaagagacggcccg |
| 178 | 207126 | tatttgaatatgtatcgtgccccctccggtgtctttgatcaggatgacatgtgccatttttcagaggacgtgcagacaggctg |
| | _x_at | gcattctagattacttttcttactctgaaacatggcctgtttgggagtgcgggattcaaaggtggtcccaccg |
| | | ctgcccctactgcaaatggcagttttaatcttatcttttggcttctgcagatggttgcaattgatcc |
| | | ttaaccaataatggtcagtcctcatctctgtcctgcttcataggtgccaccttgtgtgtttaaa |
| 179 | 207134 | tgacgcaaaataccaccttggcgcctacacgggagacgacgtccgcatcgtccgtgacgacatgctgtgtgccgggaacac |
| | _x_at | ccggagggactcatgccagggcgactccggagggcccctggtgtgcaaggtgaatggcacctggctgcaggcgggcgtggt |
| | | cagctggggcgagggctgtgcccagcccaaccggcctggcatctacacccgtgtcacctactacttggactggatccac |
| | | cactatgtccccaaaaagccgtgagtcaggcctgggttggccacctgggtcactggaggaccaacccctgctgtccaaaac |
| | | accactgcttcctacccaggtggcgactgccccccacaccttccctgccccgtcctgagtgccccttcctgtcctaagccc |
| | | cctgctctcactgagccccttcccctgtcctgaggacccttccccatcctgagcccccttccctgtcctaa |
| | | gcctgacgcctgcaccgggccctccggccctcccctgcccaggcagctggtggtgggcgct |
| 180 | 207245 | atgatcacaaaatgttgcctgttttgtttccgaaagcttgccaaaacaggaaagaagaagaaaagggattagttatatcaa |
| | _at | aagcctgaagtggaatgaccaaaagatgggactcctcctttattccagcatggagggttttaaatggaggatttccttt |
| | | ttcctgcgacaaaacgtcttttcacaacttaccctgttaagtcaaaatttattttccaggaatttaatatgtactttagt |
| | | tggaattattctatgtcaatgatttttaagctatgaaaaataataatataaaaccttatgggcttatattgaaatttat |
| | | tattctaatccaaaagttaccccacacaaaagttactgagcttccttatgtttcacacattgtatttgaacac |
| | | aaaacattaacaactccactcatagtatcaacattgattgcaaatactcagaatattttggcttcattttgagcagaat |
| 181 | 207266 | caagtatcggggctctgctatcaaggtgcaaagtccttcgtggatgcaacctcaaccatatattctacagcaccctggtg |
| | _x_at | ccgtgttaactccctcaatggagcacaccatgtcactacagcccgcatcaatgatcagccctctggcccagcagatgagt |
| | | catctgtcactaggcagcaccggaacatacatgcctgcaacgtcagctatgcaaggagcctacttgccacagtatgcac |
| | | atatgcagacgacagcggttcctgttgaggaggcaagtggtcaacagcaggtggctgtcgagacgtctaatgacc |
| | | attctccatatacctttcaa |
| 182 | 207390 | tggactggtgtcgagccaagactcggggcctacgagcacgtcgacatccagaacttctcctccagctggagtgatgggat |
| | _s_at | ggccttctgtgccctggtgcacaacttcttccctgaggccttcgactatgggcagcttagccctcagaaccgacgccaga |
| | | acttcgaggtggccttctcatctgcggagatgctggtggactatgtgcccctggtggaggtggacgacatgatgatca |
| | | tgggcaagaagcctgaccccaagtgtgtcttcacctatgtgcagtcgctctacaaccacctgcgacgccacgaactg |
| | | gcctcgcgcggcaagaatgtctagcctgcccgcccgcatggccagccagtggcaactgccgcccccactctccgggcac |
| | | cgtctcctgcctgtgcgtccgcccaccgctgccctgtctgttgcgacaccctcccccccacatacacacgcagcgttttga |
| 183 | 207392 | ggatgtgatagcattcctgctggcctgcgtggcaactgtgatatttatcatcacaaaattttgcctgttttgtttccgaaagcttgc |
| | _x_at | caaaacaggaaagaagaagaaaagagattagttatatcaaaagcctgaagtggaatgactgaaagatgggactcctcctttatt |
| | | tcagcatggagggttttaaatggaggatttcctttttcctgtgacaaaacatcttttcacaacttaccttgttaagacaaaatt |
| | | tattttccagggatttaatacgtactttagttggaattattctatgtcaatgatttttaagctatgaaaaatacaatggggggaag |
| | | gatagcatttggagatatacctaatgttaaatgacgagttactggatgcagcacgcaacatggcacatgtgtatacatatgtagc |
| | | taacccttcgttgtgcacatgtaccctaaaacttaaagtataatttaaaaaaagcaaaaaaaaaaaataccaac |
| | | tcttttttttaaaccaggaaggaaaatgtgaacatggaaacaacttctagtattggatctg |
| 184 | 207432 | gccccggagtgcagctgcggggacccgctgctcgaccccggcctgccggagcccgaggccccgccccctgcgggtcccgaaccgctt |
| | _at | accctcatccctgggcctgtcgagcccttcagcatcgtgaccatgcccgggccccggggtccggcgccaccctggctgcccagccct |
| | | attggcgaggaggaggagaatctggcctgagatcttagagcccagccccctaaggacagggaaccaggtccctgcacggcacccac |
| | | gcaggtgtcccggtctgcataagcctcgtatgcctttgtaaagtccacctacacttttgaccagctctcgctgcccgcatgtgttt |
| | | cggcgctgtgtaggggcgggagttaccagactcttggaccagcccgccctgaccaccagctctacttcccaacccccactg |
| | | cctgagaggtctctatcagtgtcctgcctgaattctttccttcaagtgaagatgtgactgactacctcctcgagttgtcatga |
| 185 | 207502 | gaacccagggagcgcgatgggctgcagggctgcgtcagggctcctgccaggagtggccgtggtcctcctgctgctgctgcagagca |
| | _at | cacagtcagtctacatccagtaccaaggcttccgggtccagctggaatccatgaagaagctgagtgacctggaggcacagtgggc |
| | | acccagcccccgcctgcaggcccagagcctcctgcccgccgtgtgccaccaccctgctctgcctcaggaccttcagcctgtctgc |
| | | gcctcgcaggaggcttccagcatcttcaagaccctgaggaccatcgctaacgacgactgtgagctgtgtgtgaacgttgcgtg |
| | | taccggctgcctctgagatagccctgggtaccctgagcccaccagggacacctcgcccttcagcccaccaccctg |
| | | gcaggcttccatccccgtccatgctcaagatgggtc |
| 186 | 207761 | ttattcttagcgtcactggtctggctttcagaattaacatacaaggttgccacacctagttctgcccagctttatgtcttttattc |
| | _s_at | cagtattccaccaaagtttgttttcctgcattccagttctcaagtcttaagataaagattgtacttgacagtttagtatatccata |
| | | aaactatttgaggtggttaaggttcttgggttcattttccttaatactttgctgaatattgtagattgtaggcaatgaaaaagt |
| | | ctactaaattaggaaaaccttgaataattaggtatcctaggtaagagcccctaaacatcaagcaatctgtgagtctgtaaagaaat |
| | | aaatattttttggattattcttatctaattccacccctgttggaagatgatttctttgttctttgcaactatggaagctgtgaaaa |
| | | tcatcacaagtgcctctgaaagcgagtgttaggttggttagagggt |
| 187 | 207961 | gtgacggtgctgaagaaggccctggatgaagagacgcggtcccatgaggctcaggtccaggagatgaggcagaaacacgcacagg |
| | _x_at | cggtggaggagctcacagagcagcttgagcagttcaagagggccaaggcgaacctagacaagaataagcagacgctggagaaa |
| | | gagaacgcagacctggccggggagctgcgggtcctgggccaggccaagcaggaggtggaacataagaagaagaagctggaggcgc |
| | | aggtgcagacactgagtttttagaaaaacatatccacggtaaccggtccctggcaattctgtttacatgaaatggggagaaagtc |
| | | accgaaatgggtgccgccggcccccactcccaattcattccctaacctgcaaacctttccaacttctcacgtcaggcctttg |
| | | agaattctttccccctctcctggtttccacacctcagacacgcacagttcaccaagtgccttctgtagtcacatgaattgaaaa |
| | | ggagacgctgctcccacggaggggagcaggaatgctgcactgtttacaccctgactgtgcttaaa |
| 188 | 207977 | tagtggccgtgaggagcatcttcagtaagaaggaaggttctgacagacaatggaactacgcctgcatgcccacgccacagagcctc |
| | _s_at | ggggaacccacggagtgctggtgggaggagatcaacagggctggcatggaatggtaccagacgtgctccaacaatgggctggtggc |
| | | aggattccagagccgctacttcgagtcagtgctggatcgggagtggcagttttactgttgtcgctacagcaagaggtgcccata |
| | | ttcctgctggctaacaacagaatatccaggtcactatggtgaggaaatggacatgatttcctacaattatgattactatatccg |
| | | aggagcaacaaccactttctctgcagtggaaagggatcgccagtggaagttcataatgtgccggatgactgaatacgactgt |
| 189 | 207980 | aatgtcatagacactgatttcatcgacgaggaagttcttatgtccttggtgatagaaatgggtttggaccgcatcaaggagctg |
| | _s_at | cccgaactctggctggggcaaaacgagtttgattttatgacggacttcgtgtgcaaacagcagcccagcagagtgagctgttga |
| | | ctcgatcgaaacccc |
| 190 | 208131 | ccccccaattgctgccaatagtggataacatgtatcactcactgccaaaaatagaaagtgaccatgaaaaataaattcgctgggga |
| | _s_at | agggggctccatgctggtgtggccaaggctgagagctctctcttctctgttacaaaacgagataagcaagtnttagaattgcctt |
| | | aaggccacactggcatctccctgaccttctccagggacagaagcaggagtaagtttctcatcccatgggcgaccagggccatctc |
| | | ctcccaccagtggcccccactcacagggagctggcaatgccctacctgcctgttctccagatggagaaacaggctctgagat |
| | | ttcacaggtatgcccaaagtcattgattttaatgattaaaaagaataaacacagtgtttcctgagtagcagtgattgttatgc |
| | | cttgctatttta |
| 191 | 208370 | gatgtattaacctacctgtgaatcatatgttgtaggaaaagctgttcccatgtctaacaggacttgaattcaaagcatgtcaagtg |
| | _s_at | gatagtagatctgtggcgatatgagagggatgcagtgcctttccccattcattcctgatggaattgttatactaggttaacatt |
| | | tgtaatttttttctagttgtaatgtgtatgtctggtaaataggtattatattttggccttacaataccgtaacaatgtttgtca |
| | | ttttgaaatacttaatgccaagtaacaatgcatgctttggaaatttggaagatggttttattctttgagaagcaaatatgtttgc |
| | | attaaatgctttgattgttcatatcaagaaattgattgaacgttctcaaaccctgtttacggtacttggtaagagggagccggtt |
| | | tgggagagaccattgcatcgctgtccaagtgtttcttgt |
| 192 | 208383 | agggcaagtgttcccaaattgacgccacctaataatcatcaccacaccgggagcagatctgaaggcacactttgatttttttaag |
| | _s_at | gataagaaccacagaacactgggtagtagctaatgaaattgagaagggaaatcttagcatgcctccaaaaattcacatccaat |
| | | gcatactttgttcaaatttaaggttactcaggcattgatcttttcagtgttttttcacttagctatgtggattagctagaatgc |
| | | acaccaaaaagatacttgagctgtatatatatatgtgtgtgtgtgtgtgtgtgtgtgtgtgtgtgcatgtatgtgcacatgt |
| | | gtctgtgtgatatttggtatgtgtatttgtatgtactgttattcaaaatatatttaatacctttggaaaatcttgggcaagat |
| | | gacctactagttaccttgaaaaaaagttgctttgttattaatattgtgct |
| 193 | 208399 | ccgagccgagcttactgtgagtgtggagatgttatcccaccatgtaaagtcgcctgcgcaggggagggctgcccatctccccaac |
| | _s_at | ccagtcacagagagataggaaacggcatttgagtgggtgtccagggccccgtagagagacatttaagatggtgtatgacaga |
| | | gcattggccttgaccaaatgttaaatcctctgtgtgtatttcataagttattacaggtataaaagtgatgacctatcatgagga |
| | | aatgaaagtggctgatttgctggtaggattttgtacagtttagagaagcgattatttattgtgaaactgttctccactccaa |
| | | ctcctttatgtggatctgttcaaagtagtcactgtatatacgtatagagaggtagataggtaggtagattttaaattgcattctga |
| | | atacaaactcatactccttagagcttgaattacatttttaaaatgcatatgtgctgtttggcaccgtgg |
| | | caagatggtatcagagagaaacccatcaattgctcaaatactc |
| 194 | 208450 | ggagctgtcaccatgacgggggaacttgaggttaagaacatggacatgaagccggggtcaaccctgaagatcacaggcagcatcgc |
| | _at | cgatggcactgatggctttgtaattaatctgggccaggggacagacaagctgaacctgcatttcaaccctcgcttcagcgaatc |
| | | caccattgtctgcaactcattggacggcagcaactgggggcaagaacaacgggaagatcacctgtgcttcagcccagggt |
| | | cagaggtcaagttcacagtgacctttgagagtgacaaattcaaggtgaagctgccagatgggcacgagctgact |
| | | tttcccaacaggctgggtcacagccacctgagctacctgagcgtaaggggcgggttcaacatgtcctc |
| 195 | 208581 | gcgtgatcctcttgatcgggaactcctgcttctccttgcctcgaaatggaccccaactgctcctgctcgcctgttggctcct |
| | _x_at | gtgcctgtgccggctcctgcaaatgcaaagagtgcaaatgcacctcctgcaagaagagctgctgctcctgctgcc |
| | | ctgtcggctgtgccaagtgtgcccagggctgcatctgcaaagggacgtcagacaagtgcagctgctgtgcctga |
| 196 | 208596 | cccgaccatcgaatcttgcgaacaacacgatacttgttaagtggctaccccaaaacgatctgcttggtcacccgatgacccgtgcc |
| | _s_at | tttatcacccatgctggttcccatggtgtttatgaaagcatatgcaatggcgttcccatggtgatgatgcccttgtttggtgatc |
| | | agatggacaatgcaaagcgcatggagactaagggagctggagtgaccctgaatgttctggaaatgacttctgaagatttaga |
| | | aaatgctctaaaagcagtcatcaatgacaaaagttacaaggagaacatcatgcgcctctccagccttcacaaggaccgcccggt |
| | | ggagccgctggacctggccgtgttctgggtggagtttgtgatgaggcacaagggcgcgccacacctgcgccccgcagcccacg |
| | | acctcacctggtaccagtaccattccttggacgtgattggtttcctcttggccgtcgtgctgacagtggccttcatcaccttt |
| | | aaatgttgtgcttatggctaccggaaatgcttggggaaaaaagggcgagttaagaaagcccacaaatccaagacc |
| 197 | 208747 | tgcagaggcctatgttttcactcctaacatgatctgtgctggaggagagaagggcatggatagctgtaaaggggacagtggtgggg |
| | _s_a | cctttgctgtacaggatcccaatgacaagaccaaattctacgcagctggcctggtgtcctgggggccccagtgtgggacctatgg |
| | | gctctacacacgggtaaagaactatgttgactggataatgaagactatgcaggaaaatagcaccccccgtgaggactaatccag |
| | | atacatcccaccagcctctccaagggtggtgaccaatgcattaccttctgttccttatgatattctcattatttcatcatgactg |
| | | aaagaagacacgagcgaatgatttaaatagaacttgattgttgagacgccttgctagaggtagagtttgatcatagaattgtgc |
| | | tggtcatacatttgtggtctgactccttggggtcctttccccggagtacctattgtagataacactatgggtggggcactccttt |
| 198 | 208763 | gggcctaaccagccttgggagtattgactggtcccttacctcttatggctaagtctttgactgtgttcatttaccaagttgaccca |
| | _s_at | gtttgtcttttaggttaagtaagactcgagagtaaaggcaaggaggggggccagcctctgaatgcggccacggatgccttgct |
| | | gctgcaaccctttccccagctgtccactgaaacgtgaagtcctgttttgaatgccaaacccaccattcactggtgctgactacata |
| | | gaatggggttgagagaagatcattttgggcttcacagtgtcatttgaaaacgtatttgttttgttttgtaattattgtggaaaac |
| | | tttcaagtgaacagaaggatggtgtcctactgtggatgagggatgaacaaggggatggctttgatccaatggagcctgggaggtg |
| | | tgcccagaaagcttgtctgtagcgggttttgtgagagtgaacactttccactttttgacaccttatcctgatgtatggttc |
| 199 | 208788 | agatgtgtttagaacctcttgtttaaaaataatagactgcttatcataaaatcacatctcacacatttgaggcagtggtcaaacag |
| | _at | gtaaagcctatgatgtgtgtcattttaaagtgtcggaatttagcctctgaataccttctccattgggggaaagatattcttggaac |
| | | cactcatgacatatcttagaaggtcattgacaatgtataaactaattgttggtttgatatttatgtaaatatcagtttaccatg |
| | | ctttaattttgcacattcgtactatagggagcctattggttctctattagtcttgtgggttttctgtttgaaaaggagtcat |
| | | ggcatctgtttacatttaccttatcaaacctagaatgtgtatatttataaatgtatgtcttcattgctaggtactaatttgcagat |
| | | gtctttacatatttcaatacagaaactataacattcaatagtgtgctgtcaaagtgtgcttagctcacctggatatacctacattg |
| 200 | 208789 | gaagggagtgttgctcccagtccagaggcctgattctgttcggactgggttctcaagacacgaccaggttctcaagacacgagtcc |
| | _at | ccttgttcctccccattaaagggggtttgtcagaagcaagaacagcccctctccccagtcacagcctgaagggaggccccgaga |
| | | gcttcctccttccccccacctgctccttaccttctctgccctgctttttagaactgcagttcattgttttaagggattggggga |
| | | gggagcctggggacacaaaccttttatacaatacaaagctttgctttttttttttttttnnnannncttttccctttctcggtt |
| | | ctcttctctcctctgaatggctgaagacccctctgccgagggaggttggggattgtgggacaaggtcccttggtgctgatggcc |
| | | tgaaggggcctgagctgtgggcagatgcagttttctgtgggcttggggaacctctcacgttgctgtgtcctc |
| | | tgagcagcccgaccaataaacctgcttt |
| 201 | 208791 | ggctgcctgcggatgaaggaccagtgtgacaagtgccgggagatcttgtctgtggactgttccaccaacaacccctcccaggcta |
| | _at | agctgcggcgggagctcgacgaatccctccaggtcgctgagaggttgaccaggaaatataacgagctgctaaagtcctaccagtg |
| | | gaagatgctcaacacctcctccttgctggagcagctgaacgagcagtttaactgggtgtcccggctggcaaacctcacgcaa |
| | | ggcgaagaccagtactatctgcgggtcaccacggtggcttcccacacttctgactcggacgttccttccggtgtcact |
| | | gaggtggtcgtgaagctctttgactctgatcccatcactgtgacggtccctgtagaagtctccaggaagaacccta |
| 202 | 208792 | agcagctgaacgagcagtttaactgggtgtcccggctggcaaacctcacgcaaggcgaagaccagtactatctgcgggtcacca |
| | _s_at | cggtggcttcccacacttctgactcggacgttccttccggtgtcactgaggtggtcgtgaagctctttgactctgatccca |
| | | tcactgtgacggtccctgtagaagtctccaggaagaaccctaaatttatggagaccgtggcgga |
| | | gaaagcgctgcaggaataccgcaaaaagcaccgggaggagtgagatgtggatgttgc |
| 203 | 208894 | cgatcaccaatgtacctccagaggtaactgtgctcacgaacagccctgtggaactgagagagcccaacgtcctcatctgttt |
| | _at | catagacaagttcacccca |
| 204 | 208920 | gatataccatgtattcacactttaaaaaatacacatagcagagtatcggaaagtatgtaccaaaacattgatctggctaccactgg |
| | _at | gagtcaggatcatgagttcatcttcttcntncttnnnactgcttttccaaacattctccagtaagcaggtactacatttataat |
| | | ggaaggaattttttaaaaaattttaagctgtacactttcaagtaagatctgaattctaatgctggcttgtgcctcttactat |
| | | gtggtttggtcattatagataatgcccagtttcagtttccccatctttgaaaaggagataatatgttatctcttgggagtag |
| | | ttctgaatatgaagttgtttgatacaagaagcactgacaatgtttctgtttcttaaatttaaaactggcctggtttgcctttt |
| | | ttatcaagagagcttaacagataaaaaatgaaattagtctattttctacttgccagcagagtatctgtcttattttaggatgc |
| | | agtgtgaaacttaccattcaactgaacaac |
| 205 | 209047 | gaaggctggattctatctacataagtcctttcaattccaccagggccagagcagctccaccactgtgcacttagccatgatggcaa |
| | _at | cagaaaccaagagacacaattacgcaggtatttagaagcagagggacaaccagaaggcccttaactatcaccagtgcatcacatc |
| | | tgcacactctcttctccattccctagcaggaacttctagctcatttaacagataaagaaactgaggcccacggtttcagctag |
| | | acaatgatttggccaggcctagtaaccaaggccctgtctctggctactccctggaccacgaggctgattcctctcatttcca |
| | | gcttctcagtttctgcctgggcaatggccaggggccaggagtggggagagttgtgatggaggggagaggggtcacacccacccc |
| | | ctgcctggttntaggctgctgcacaccaaggccctgcatctgtctgctctgcatatatgt |
| 206 | 209074 | agacagctgtttagggtcttctcccctcacccatgctttcatcatcccctccgcacagcctccccgtccaggccttctaaccaca |
| | _s_at | cctacccagggctgccgcattcctgcactcagaagtctgcagcggtgcctcacaaacttgattgtgcataaaaatcactggggat |
| | | cttgttaatacagattctaactcaatagatctgggagatcctgcatttctaacaagctcccaggtaaggcggaggctgctggtgt |
| | | gaggaccatgctgtgagcagcagggcgagagtgcccagggctgatatatattggaaatatcacccctgaagccatcgctggcc |
| | | cccacctcctgtggactgatgccccagggattcccaccccacttctgcaaccccaggtatccttcattatccaccccatcccag |
| | | actcccaccccagggattgcccgtgaagactttggcctagcaaattgtgttggttatgtgagtgttgttttaatcagagatgta |
| | | catgattgccaatctgcatttcttaccagtgtgaccacac |
| 207 | 209101 | atcatcagatcgactcttatacgagtaatatgcctgctatttgaagtgtaattgagaaggaaaattttagcgtgctcactgacct |
| | _at | gcctgtagccccagtgacagctaggatgtgcattctccagccatcaagagactgagtcaagttgttccttaagtcagaacagcag |
| | | actcagctctgacattctgattcgaatgacactgttcaggaatcggaatcctgtcgattagactggacagcttgtggcaagtga |
| | | atttgcctgtaacaagccagattttttaaaatttatattgtaaatattgtgtgtgtgtgtgtgtgtgtatatatatatatata |
| | | tgtacagttatctaagttaatttaaagttgtttgtgcctttttatttttgtttttaatgctttgatatttcaatgttagcct |
| | | caatttctgaacaccataggtagaatgtaaagcttgtctgatcgttcaaagcatgaaatggatacttatatggaa |
| | | attctgctcagatagaatgacagtccgtcaaaacagattgtttgcaaaggggaggcatcagtgtcttg |
| 208 | 209114 | tacaccacaatggctgagcacttcctgacgttgctggtagtgcctgccatcaagaaagattatggttcccaggaagacttcactca |
| | _at | agtgtggaacaccaccatgaaagggctcaagtgctgtggcttcaccaactatacggattttgaggactcaccctacttcaaag |
| | | agaacagtgcctttcccccattctgttgcaatgacaacgtcaccaacacagccaatgaaacctgcaccaagcaaaaggctcacg |
| | | accaaaaagtagagggttgcttcaatcagcttttgtatgacatccgaactaatgcagtcaccgtgggtggtgtggcagctgg |
| | | aattgggggcctcgagctggctgccatgattgtttccatgtatctgtactgcaatctacaataagtccacttctgcctctgcca |
| | | ctactgctgccacatgggaaactgtgaagaggcaccctgggcaagcagcagtgattgggggaggggacaggatctaacaatgtc |
| | | acttgggccagaatggacctggcctttctgctcccagacttgggggctag |
| 209 | 209116 | gcagttatgggcaaccctaaggtgaaggctcatggcaagaaagtgctcggtgcctttagtgatggcctggctcacctggacaa |
| | _x_at | cctcaagggcacctttgccacactgagtgagctgcactgtgacaagctncangtggatcctgagaacttcaggctnctnggca |
| | | acgtgntngtctgngtgctggcccatcactttggcaaagaattcaccccaccag |
| 210 | 209138 | tctctgggctccaggctgaggacgaggctgattattactgctgctcatatgcaggtagttacactgtggttttcggcggagggacc |
| | _x_at | aaactgaccgtcctaggtcagcccaaggctgccccctcggtcactctgttcccgccctcctctgaggagcttcaagccaacaa |
| | | ggccacactggtgtgtctcataagtgacttctacccgggagccgtgacagtggcctggaaggcagataggagccccgtcaaggcgg |
| | | gagtggagaccaccacaccctccaaacaaagcaacaacaagtacgcggccagcagctatctgagcctgacgcctga |
| | | gcagtggaagtcccacagaagctacagctgccaggtcacgcatgaagggagcaccgtggagaagacagtggcccctacag |
| | | aatgtt |
| 211 | 209147 | tttggtgcagctgctagtcagtccctgactgacattgccaagtattcaataggcagactgcggcctcacacttggatgtttgtga |
| | _s_at | tccagattggtcaaaaatcaactgcagcgatggttacattgaatactacatatgtcgagggaatgcagaaagagttaaggaagg |
| | | caggttgtccttctattcaggccactcttcgttttccatgtactgcatgctgtttgtggcactttatcttcaagccaggatgaa |
| | | gggagactgggcaagactcttacgccccacactgcaatttggtcttgttgccgtatccatttatgtgggcctttctcgagttt |
| | | ctgattataaacaccactggagcgatgtgttgactggactcattcagggagctctggttgcaatattagttgctgtatatgtat |
| | | cggatttcttcaaagaaagaacttcttttaaagaaagaaaagaggaggactctcatacaactc |
| | | tgcatgaaacaccaacaactgggaatcactatccgagcaatcaccagc |
| 212 | 209156 | gcacgccatcaatgccatcgtgcgcagcccgcgtggcggggcccggaggcacgcagagctgtccttcgtgttcctcacggacggcg |
| | _s_at | tcacgggcaacgacagtctgcacgagtcggcgcactccatgcgcaacgagaacgtggtacccaccgtcctggccttgggcagc |
| | | gacgtggacatggacgtgctcaccacgctcagcctgggtgaccgcgccgccgtgttccacgagaaggacta |
| | | tgacagcctggcgcaacccggcttcttcgaccgcttcatccgctggatctgctagcgccgccgcccg |
| | | ggccccgcagtcgagggtcgtgagcccaccccgtccatggtgctaagcgggccc |
| 213 | 209167 | tgtttcctcatagctagttctcaagctgcatgtaagattttaacgggaagagaaaataggcctggacctgaaggtctcaaatat |
| | _at | gttgagaagaaagtatgaactataaggaacttgagatgtagatttattttgcaggaaatacgaggaaaataggaaagaagtg |
| | | tttgccgcatcaagtgtaaagaatgatactgaggatttttacattttatgaaatgaaataatggcatttacaaaatgaaaaat |
| | | gtagtttcacaactaagttttgttaacagagactgcatgctttgcttatagttcttaattttggttttgacattcatttaat |
| | | tttttccatgttaaatatgtagtttaattatttactcaaaataaacattgttcatgcttttaggcctttgggggaattgattt |
| | | ttatccacaggtagaaaatggtctttgcacacactacacttatttcaaatatacaatgtgctcccgaactttcgcattagtctt |
| 214 | 209170 | gaaatatcactgacactccagactaaaggagagtctaggtttctgcaattttgttacagtaatttgtaaatagctttagtaaactc |
| | _s_at | accttgcatggtagattaataagatgacttactgtacatgaattacacaataatgagatctggtggctatttccacattttga |
| | | aaaggattcagttatttactgacagtggtgagcatcctttttaaaataatgttctgatacttaaacattagagagcagtatctt |
| | | taaatgaattattaacactttggaatacttacattttctgttatttttgattgcctgataaccagtttcaatgatgaaaatgaa |
| | | aacaagtgctgaagatgaaatggaagagaaccgttttaatctggattttgttttgtcacacctggaaaatactttgcaaatatg |
| | | ttctaaattgaaaacaatttttttatgatcacatggttcactaccaaatgaccctcaaataagccagatg |
| 215 | 209191 | gatagtcggaatagagccgccccaactcagatcctacaacacgcaagttccttcttgaaccctggtgcctcctaccctatggccct |
| | _at | gaatggtgcactggtttaattgtgttggtgtcggcccctcacaaatgcagccaagtcatgtaattagtcatctggaacaaagac |
| | | taaaaacagcagagaattgcgggttctacccagtcagaagatcacaccatggagactttctactagaggacttgaaagagaa |
| | | ctgaggggccacaaaataaacttcaccttccattaagtgttcaagcatgtctgcaaattaggag |
| | | ggagttagaaacagtctttttcatcctttgtgatgaagcctgaaattgtgccgtgttgccttatatgaatatg |
| 216 | 209209 | tgtctccccgctatctaaaaaagtataagaacaagcagataacagcgagaatcttggaggcccatcagaatgtagctcagatgag |
| | _s_a | tctaattgaagccaagatgagatttattcaagcttggcagtcactacctgaatttggcatcactcacttcattgcaaggttcca |
| | | agggggcaaaaaagaagaacttattggaattgcatacaacagactgattcggatggatgccagcactggagatgcaattaaaaca |
| | | tggcgtttcagcaacatgaaacagtggaatgtcaactgggaaatcaaaatggtcaccgtagagtttgcagatgaagtacgattgt |
| | | ccttcatttgtactgaagtagattgcaaagtggttcatgaattcattggtggctacatatttctctcaacacgtgcaaaagacca |
| | | aaacgagagtttagatgaagagatgttctacaaacttaccagtggttgggtgtgaatagaaatactgtttaatgaaactccacgg |
| | | ccataa |
| 217 | 209210 | aaaatgctattagtccgtcgtgcttnatttgtttttgtccttgaataagcatgttatgtatatngtctcgtgtttttatttttac |
| | _s_at | accatattgtattacacttttagtattcaccagcataancactgtctgcctaaaatatgcaactctttgcattacaatatg |
| | | aagtaaagttctatgaagtatgcattttgtgtaactaatgtaaaaacacaaattttataaaattgtacagttttttaaaaac |
| | | tactcacaactaggagatggcttaaatgtagcaatctctgcgttaattaaatgcctttaagagatataattaacgtgcagttt |
| | | taatatctactaaattaagaatgacttcattatgatcatgatttgccacaatgtccttaactctaatgcctggactggcc |
| | | atgttctagtctgttgcgctgttacaatctgtattggtgctagtcagaaaattcctagctca |
| | | catagcccaaaagggtgcgagggagaggtggattaccagtattgttcaataatccatggttca |
| 218 | 209283 | gaccagttcttcggagagcacctgttggagtctgatcttttcccgacgtctacttccctgagtcccttctaccttcggccaccctc |
| | _at | cttcctgcgggcacccagctggtttgacactggactctcagagatgcgcctggagaaggacaggttctctgtcaacctggatgtg |
| | | aagcacttctccccagaggaactcaaagttaaggtgttgggagatgtgattgaggtgcatggaaaacatgaagagcgccaggat |
| | | gaacatggtttcatctccagggagttccacaggaaataccggatcccagctgatgtagaccctctcaccattacttcatccctgtc |
| | | atctgatggggtcctcactgtgaatggaccaaggaaacaggtctctggccctgagcgcaccattcccatcacccgtgaaaagaagcc |
| | | tgctgtcaccgcagcccccaagaaaaagatgccctttcttgaattgcattttttaaaacaagaaagtttccccaccagtgaatgaa |
| 219 | 209301 | tgaatcttcgggtgtttccctttagctaagcacagatctaccttggtgatttggaccctggttgctttgtgtctagttttctagacc |
| | _at | cttcatctcttacttgatagacttactaataaaatgtgaagactagaccaattgtcatgcttgacacaactgctgtggctggttg |
| | | gtgctttgtttatggtagtagtttttctgtaacacagaatataggataagaaataagaataaagtaccttgactttgttca |
| | | cagcatgtagggtgatgagcactcacaattgttgactaaa |
| 220 | 209312 | ctgtgtatccttcaaagacccagcccctgcagcaccataacctcctggtctgttctgtgagtggtttctatccaggcagcattgaa |
| | _x_at | gtcaggtggttccggaatggccaggaagagaagactggggtggtgtccacaggcctgatccacaatggagactggaccttccag |
| | | accctggtgatgctggaaacagttcctcggagtggagaggtttacacctgccaagtggagcacccaagcgtgacaagccctctc |
| | | acagtggaatggagagcacggtctgaatctgcacagagcaagatgctgagtggagtcgggggctttgtgctggg |
| | | cctgctcttccttggggccgggctgttcatctacttcaggaatcagaaaggacactctggacttcagcca |
| 221 | 209335 | agtcaaatgccaaacactagctctgtattaatccccatcattactggtaaagcctcatttgaatgtgtgaattcaatacaggctat |
| | _at | gtaaaattatactaatgtcattattttgaaaaaataaatttaaaaatacattcaaaattannannnnanacaagcttaattg |
| | | ttaatattccctaaacacaattttatgaagggagaagacattggtttgttgacaataacagtacatcttttcaagttctcagcta |
| | | tttcttctacctctccctatcttacatttgagtatggtaacttatgtcatctatgttgaatgtaagcttataaagcacaaa |
| | | gcatacatttcctgactggtctagagaactgatgtttcaatttacccctctgctaaataaa |
| 222 | 209357 | ggcaaactgcttaatcttgtggattttgtagatggtttcaaatgactgaactgcattcagatttacgagtgaaaggaaaaattgca |
| | _at | ttagttggttgcatgaactttgaagggcagatattactgcacaaactgccatctcgcttcatttttttaactatgcatttga |
| | | gtacagactaatttttaaaatatgctaaactggaagattaaacagatgtggcccaaactgttctggatcaggaaagtcata |
| | | ctgttcactttcaagttggctgtcccccccgccgcccccccccacccccatatgtacagatgataatagggtgtggaat |
| | | gtcgtcagtggcaaacatttcacagattattttgtttctgtcttcaacatttttgacactgtgctaat |
| 223 | 209373 | ctcctccatgagtctgacatctcggaaactgagcagctgccggacgcctgggtcaggaatccaagaccccacctcttaaggactgg |
| | _at | ttcctcagaaagcaccctcagggaaaaaggtgaaaacattacatccgtggattctcctgccacaaccgcattggaagaaaaggct |
| | | gccgcaacatctcagcgaggagtgaaggacccatgtcccaggaaccgcgctgcgccacctgcactcacccccctcacattctc |
| | | ttaagcacccggtggccctccgaggcctggcggaatggtggtgcccacggggttgggcaagggctcaccaggacctcaacgg |
| | | gcaaagttgtgcacactaaaatatcaaatcaaggtgcttggttttaaagtaaatgatttctaaagaaagctgtgttcttc |
| | | tgttgacccagacgaatagggcacagccctgtaactgcacgtgccttctgtcattgggaatg |
| 224 | 209374 | caccatcacgtgcctggtgacgggcttctctcccgcggacgtcttcgtgcagtggatgcagagggggcagcccttgtccccgga |
| | _s_at | gaagtatgtgaccagcgccccaatgcctgagccccaggccccaggccggtacttcgcccacagcatcctgaccgtgtccgaag |
| | | aggaatggaacacgggggagacctacacctgcgtggtggcccatgaggccctgcccaacagggtcaccgagaggaccgtggac |
| | | aagtccacc |
| 225 | 209436 | ttcttttttatattgtcctccacctccatcattttcaataaaagatagggcttttgctcccttgttcttggagggaccattattac |
| | _at | atctctgaactacctttgtatccaacatgttttaaatccttaaatgaattgctttctcccaaaaaaagcacaatataaagaaac |
| | | acaagatttaattatttttctacttggggggaaaaaangtcctcatgtagaagcacccacttttgcaatgttgttctaagctatc |
| | | tatctanctctcagcccatgataaagttccttaagctggtgattcctaatcaaggacaagccaccctagtgtctcatgtttg |
| | | tat |
| 226 | 209457 | ggaagcacaatttccaccttattttttgaactttggcagtttcaatgtctgtctctgttgcttcggggcataagctgatcaccgtc |
| | _at | tagttgggaaagtaaccctacagggtttgtagggacatgatcagcatcctgatttgaaccctgaaatgttgtgtagacaccctct |
| | | tgggtccaatgaggtagttggttgaagtagcaagatgttggcttttctggattttttttgccatgggttcttcactgacttggc |
| | | actttggcatgattcttagtcatacttgaacttgtctcattccacctcttctcagagcaactcttcctttgggaaaagagtt |
| | | cttcagatcatagaccaaaaaagtcataccttcgaggtggtagcagtagattccaggaggagaagggtacttgctaggtatcct |
| | | gggtcagtggcggtgcaaactggtttcctcagctgcctgtccttctgtgtgcttatgtctcttgtgacaattgttttcctccct |
| 227 | 209458 | agagaacccaccatggtgctgtctcctgccgacaagaccaacgtcaaggccgcctggggtaaggtcggcgcgcacgctggcgagt |
| | _x_at | atggtgcggaggccctggagaggatgttcctgtccttccccaccaccaagacctacttcccgcacttcgacctgagccacggc |
| | | tctgcccaggttaagggccacggcaagaaggtggccgacgcgctgaccaacgccgtggcgcacgtggacgacatgcccaacg |
| | | cgctgtccgccctgagcgacctgcacgcgcacaagcttcgggtggacccggtcaacttcaagctcctaagccactgcctg |
| | | ctggtgaccctggccgcccacctccccgccgagttcacccctgcggtgcacgcctccctggacaagttcctggcttctgtg |
| | | agcaccgtgctgacctccaaataccgttaagctg |
| 228 | 209496 | tggaggaatttcacaagcacccgcccgtgcagtgggccttccaggagaccagtgtggagagcgccgtggacacgcccttcccagctg |
| | _at | gaatatttgtgaggctggaatttaagctgcagcagacaagctgccggaagagggactggaagaaacccgagtgcaaagtcaggccc |
| | | aatgggaggaaacggaaatgcctggcctgcatcaaactgggctctgaggacaaagttctgggccggttggtccactgccccata |
| | | gagacccaagttctgcgggaggctgaggagcaccaggagacccagtgcctcagggtgcagcgggctggtgaggacccccac |
| | | agcttctacttccctggacagttcgccttctccaaggccctgccccgcagctaagccagcactgagctgcgtggtgcctc |
| | | caggaccgctgccggtggtaaccagtggaagaccccagcccccagggagaggaccccgttctatccccagccatgataata |
| 229 | 209498 | tttgtcaggaaaccttggcctctgctaaggtgtatttggtccttgagaagtgggagcaccctacagggacactatcactcatgctg |
| | _at | gtggcattgtttacagctagaaagctgcactggtgctaatgccccttggggaaatggggctgtgaggaggaggattataactta |
| | | ggcctagcctcttttaacagcctctgaaatttatcttttcttctatggggtctataaatgtatcttataataaaaagg |
| | | aaggacaggaggaagacaggcaaatgtacttctcacccagtcttctacacagatggaatctctttggggctaaga |
| | | gaaaggttttattctatattgcttacctgatctcatgttaggcctaagaggctttctccaggaggattagcttggag |
| | | ttctctatactcaggtacctctttcagggttttctaaccctgacacggactgtgcatactttccc |
| 230 | 209612 | actggatgcgttaataacccatgttttaccttttgaaaaaataaatgaaggatttgacctgcttcactctgggaaaagtatccgt |
| | _s_at | accgtcctgacgttttgaggcaatagagatgccttcccctgtagcagtcttcagcctcctctaccctacaagatctggagcaac |
| | | agctaggaaatatcattaattcagctcttcagagatgttatcaataaattacacatgggggctttccaaagaaatggaaattg |
| | | atgggaaattatttttcaggaaaatttaaaattcaagtgagaagtaaataaagtgttgaacatcagctggggaattgaagccaa |
| | | caaaccttccttcttaaccattctactgtgtcacctttgccattgaggaaaaatattcctgtgacttcttgcatttttggtatc |
| | | ttcataatctttagtcatcgaatcccagtggaggggacccttttacttgccctgaa |
| | | catacacatgctgggccattgtgattgaagtcttctaactctgtctcagt |
| 231 | 209613 | gcagatttcttgcttcatatgacaaagcctcaattactaattgtaaaaactgaactattcccagaatcatgttcaaaaaatctgta |
| | _s_at | atttttgctgatcgaaagtgcttcattgactaaacagtattagtttgtggctataaatgattatttagatgatgactgaaaatgt |
| | | gtataaagtaattaaaagtaatatggtggctttaagtgtagagatgggatggcaaatgctgtgaatgcagaatgtaaaattgg |
| | | taactaagaaatggcacaaacaccttaagcaatatattttcctagtagatatatatatacacatacatatatacacatatacaa |
| | | atgtatatttttgcaaaattgttttcaatctagaacttttctattaactaccatgtcttaaaatcaagtctataatcctagcatt |
| | | agtttaatattttgaatatgtaaacacctgtgttaatgctttgttaatgcttttcccactctcatttgtta |
| | | atgctttcccactctcgggaaggatttgcattttgagctttatctctaaatgtgacatgca |
| 232 | 209621 | ggatgccgctctgtgacaaatgtggcagtggcatagtcggtgctgtggtgaaggcgcgggataagtaccggcaccctgagtgctt |
| | _s_at | cgtgtgtgccgactgcaacctcaacctcaagcaaaagggctacttcttcatagaaggggagctgtactgcgaaacccacgcaag |
| | | agcccgcacaaaacccccaagaggctatgacacggtcactctgtatcccaaagcttaagtctctgcaggcgtggcacacg |
| | | cacgcacccacccacgcgcacttacacgagaagacattcatggctttgggcagaaggattgtgcagattgtcaactccaaa |
| | | tctaaagtcaaggctttagacctttatcctattgtttattgaggaaaaggaatgggaggcaaatgcctgctatgtgaaaaa |
| | | aacatacacttagctatgttttgcaactctttttggggctag |
| 233 | 209651 | ttcctgaagctcttcggctgacagcccgctcggctcgccctctcccccggaggccgcgccctcccggaaaagccgggtcctccag |
| | _at | accccgaggccttgctctcagagcgggaggccccacccactggagagccccgcccctaaggtactatgagttctcaggggtcaa |
| | | gttcagaaacggcccagccagacctaaacccacacgcccacaaagtggattgcacacagacaagaactcccgtgcgggcct |
| | | ccactctattcccacccttgagggagcccccttactgggggagggtccttgcaattccagcgaatcggaggccag |
| | | gccaggacgtccttgctccctgcaccctcactgttctgtgcactttttctacctacataaacacacgcatt |
| 234 | 209656 | gcagggctagttattccgatttcttgcacaattatttagctttttgtaagttcaacatgtaaattttaaagacataaatatagagag |
| | _s_at | acttatgtgtttgaatataaatgatatatatggattagcatgtacctgtatattattaaacatgcaatgaactgactggtaagtga |
| | | cgtctaattgtatggctagcaatgtaatttattcagactgtatttttgtacagagcagtgcactctaacctatgcctctgtgtcc |
| | | tctttaatgcctaaagctgtgcctagaaatttcatctgtcttaaaagtaaaatataattcatgctgtttatgctattagtttctgt |
| | | actgctattctatatttattatttttaaatatatgacatgtttactacttaaacatgaattcatggtatcctggttatttttttta |
| | | agtcatctgggggaaaacctgtttatcactccagtgattttgagtttgcagtttcacaatcagttcttcat |
| 235 | 209667 | caggccttttccactttgagggaggtgcttcgaagaatgttgcccacacctaagtgttagaagcctatgtccgttcatccctga |
| | _at | gaggtctgaa |
| 236 | 209668 | tggcggactccatgtttgtgatccctgcactccaagtagcacattttcagtgttcccgggcccctgtgtacttctacgagttccag |
| | _x_at | catcagcccagctggctcaagaacatcaggccaccgcacatgaaggcagaccatggtgatgagcttccttttgttttcagaagtt |
| | | tctttgggggcaactacattaaattcactgaggaagaggagcagctaagcaggaagatgatgaagtactgggccaactttgcgag |
| | | aaatgggaacccgaatggcgagggtctgccacactggccgctgttcgaccaggaggagcaatacctgcagctgaacctacagcct |
| | | gcggtgggccgggctctgaaggcccacaggctccagttctggaagaaggcgctgccccaaaagatccaggagctcgaggagcctga |
| | | agagagacacacagagctgtagctccctgtgccggggaggagggggtgggttcgctgacaggcgagggtcagcctgctgtg |
| 237 | 209687 | gagagctcgctttgagtgactgggttttgtgattgcctctgaagcctatgtatgccatggaggcactaacaaactctgaggtttc |
| | _at | cgaaatcagaagcgaaaaaatcagtgaataaaccatcatcttgccactaccccctcctgaagccacagcagggtttcaggttcc |
| | | aatcagaactgttggcaaggtgacatttccatgcataaatgcgatccacagaaggtcctggtggtatttgtaactttttgcaag |
| | | gcatttttttatatatatttttgtgcacatttttttttacgtttctttagaaaacaaatgtatttcaaaatatatttatagtc |
| | | gaacaattcatatatttgaagtggagccatatgaatgtcagtagtttatacttctctattatctcaaactactggcaatttgta |
| | | aagaaatatatatgatatataaatgtgattgcagcttttcaatgttagccacagt |
| 238 | 209735 | tgtggtttctgtagcaacacttctcatgaccatctgttttgtgtttatgatgattttttcaggtctgttggtcaatctcacaacc |
| | _at | attgcatcttggctgtcatggcttcagtacttcagcattccacgatatggatttacggctttgcagcataatgaatttttggga |
| | | caaaacttctgcccaggactcaatgcaacaggaaacaatccttgtaactatgcaacatgtactggcgaagaatatttggtaaag |
| | | cagggcatcgatctctcaccctggggcttgtggaagaatcacgtggccttggcttgtatgattgttattttcctcacaattg |
| | | cctacctgaaattgttatttcttaaaaaatattcttaaatttccccttaattcagtatgatttatcctcacataaaaaagaa |
| | | ggcactttgattgaagtattcaatcaagtttttttttgttttctgttcccttgccatcacactgttgcacagca |
| 239 | 209763 | gaacacgcagagagtttccctagatatactcctgcctccaggtgctgggacacacctttgcaaaatgctgtgggaagcaggagctg |
| | _at | gggagctgtgttaagtcaaagtagaaaccctccagtgtttggtgttgtgtagagaataggacatagggtaaagaggccaagctgcc |
| | | tgtagttagtagagaagaatggatgtggttcttcttgtgtatttatttgtatcataaacacttggaacaacaaagaccataagc |
| | | atcatttagcagttgtagccattttctagttaactcatgtaaacaagtaagagtaacataacagtattaccctt |
| | | tcactgttctcacaggacatgtacctaattatggtacttatttatgtagtcactgtatttctggattttt |
| 240 | 209791 | ctccagccccagagctgaaaacaccaagtgcctatttgagggtgtctgtctggagacttagagtttgtcatgtgtgtgtgtgtnn |
| | _at | ttggttaatgtgggtttatgggttttctttctttttttttttttttttttnnagtctacattagggggaagtgagcgcctccc |
| | | atgtgcagacagtgtgtctttatagatttttctaaggctttccccaatgatgtcggtaatttctgatgtttctgaagttccca |
| | | ggactcacacacccgttcccatctcacttgcccacccagtgtgacaaccctcggtgtggatatacccccgtggactcatggct |
| | | cttccccacccccactttctataaatgtaggcctagaatacgcttctctgttgcaaaactcagctaagttc |
| | | ctgcttccaccttgatgttgaaatatcttatgtaagagggcaggggatgtcgtgaagatggc |
| 241 | 209868 | ggaatgacacttacttacgacccaactacagctgctatacagaacggattttatccttcaccatacagtattgctacaaaccgaat |
| | _s_at | gatcactcaaacttctattacaccctatattgcatctcctgtatctgcctaccaggtgcaaagtccttcgtgga |
| | | tgcaacctcaaccatatattctacagcaccctggtgccgtgttaactccctcaatgga |
| | | gcacaccatgtcactacagcccgcatcaatgatcagccctctggcccagcagatgagtcatctg |
| | | tcactaggcagcaccggaacatacatgcctgcaacgtcagctatgcaaggagcctacttgccacagtatg |
| 242 | 209948 | ggaacgaaaccagcgtcctattccagcgcctctacgggccccaggccctcctcttctccctcttctggcccaccttcctgctga |
| | _at | ccggtggcctcctcattatcgccatggtgaagagcaaccagtacctgtccatcctggcggcccagaagtagagccatcca |
| | | tccatgccataccacttgtcagggcacaggggactggctgggcccccagggctgctccccacttgcagcacaatgccttctcca |
| | | cctgccctcccactcttccagtccaatccacgctgtcttctgttgcaggactaacctttgagaaatccttttgtgaagtcattg |
| | | cctgctcaagaatgtacagtggctccccaatgccttggagccataaggccagccagttctagctctctatta |
| | | cctgtccccactcaactgactcatacctgtttccggctgcatcactatgtgcccca |
| | | cagagaacgatgatcgtcacctctgtgcctgagttctccctgttgtctcaaagcggtacc |
| 243 | 210084 | ccggtcagcaggatcatcgtgcacccacagttctacatcatccagactggagcggatatcgccctgctggagctggaggagcccg |
| | _x_at | tgaacatctccagccgcgtccacacggtcatgctgccccctgcctcggagaccttccccccggggatgccgtgctgggtca |
| | | ctggctggggcgatgtggacaatgatgagcccctcccaccgccatttcccctgaagcaggtgaaggtccccataatggaaa |
| | | accacatttgtgacgcaaaataccaccttggcgcctacacgggagacgacgtccgcatcatccgtgacgacatgctgtgtgcc |
| | | gggaacacccggagggactcatgccagggcgactctggagggcccctggtgtgcaaggtgaatggcacctggctacaggcg |
| | | ggcgtggtcagctgggacgagggctgtgcccagcccaaccggcctggcatctacacccgtgtcacctactacttggactgga |
| | | tccaccactatgtccccaaaaagccgtgagtcaggcctggggtgt |
| 244 | 210107 | ggccaaatcaccgacctgaaggcggaaattcacgggggcagtctcattaatctgacttggacagctcctggggatgattatgacc |
| | _at | atggaacagctcacaagtatatcattcgaataagtacaagtattcttgatctca |
| | | gagacaagttcaatgaatctcttcaagtgaatactactgctctcatcccaaaggaagccaactctgaggaagt |
| | | ctttttgtttaaaccagaaaacattacttttgaaaatggcacagatcttttcattgctattcaggctgttgataaggtcgat |
| | | ctgaaatcagaaatatccaacattgcacgagtatctttgtttattcctccacagactccgccaga |
| | | gacacctagtcctgatgaaacgtctgctccttgtcctaatattcatatcaacagcaccattcctggcattcacat |
| | | tttaaaaattatgtggaagtggataggagaactgcagctgtcaatagcctagggc |
| 245 | 210133 | accccttcagcgactagagagctacaggagaatcaccagtggcaaatgtccccagaaagctgtgatcttcaagaccaaactggc |
| | _at | caaggatatctgtgccgaccccaagaagaagtgggtgcaggattccatgaagtatctg |
| | | gaccaaaaatctccaactccaaagccataaataatcaccatttttgaaaccaaaccagagcct |
| | | gagtgttgcctaatttgttttcccttcttacaatgcattctgaggtaacctcattatcagtccaaagggcatgggttttatta |
| | | tatatatatatattnttttaaaaaaaaacgtattgcatttaatttattgaggctttaaaacttatcctccatgaatatc |
| | | agttatttttaaactgtaaagctttgtgcagattctttaccccctgggagccccaattcgatcccct |
| | | gtcacgtgtgggcaatgttccccc |
| 246 | 210139 | tgtgaagctttacgcgcacacggacaaaatgcccaaactggagcccttgcaaaaacacggcttgtggcattggcatacttgccct |
| | _s_at | tacaggtggagtatcttcgtcacacatctaaatgagaaatcagtgacaacaagtctttga |
| | | aatggtgctatggatttaccattccttattatcactaatcatctaaacaactcactggaaatccaattaa |
| | | caattttacaacataagatagaatggagacctgaataattctgtgtaatataaatggtttataactgattttgtacctagcta |
| | | ggctgctattattactataatgagtaaatcataaagccttcatcactcccacatttttcttacggtcg |
| | | gagcatcagaacaagcgtctagactccttgggaccgtgagttcctagagcttggctgggtctaggctgtt |
| | | ctgtgcctccaaggactgtctggcaatgacttgtattggccaccaactgtagatgtatatatggtgcccttctgatgctaa |
| | | gactccagaccttttgt |
| 247 | 210298 | tctactgcgtgacttgccatgagaccaagtttgccaagcattgcgtgaagtgcaacaaggccatcacatctggaggaatcactt |
| | _x_at | accaggatcagccctggcatgccgattgctttgtgtgtgttacctgctctaagaagctggctgggca |
| | | gcgtttcaccgctgtggaggaccagtattactgcgtggattgctacaagaactttgtggccaa |
| | | gaagtgtgctggatgcaagaaccccatcactgggaaaaggactgtgtcaagagtgagccgcccagtctctaaagctaggaag |
| | | cccccagtgtgccacgggaaacgcttgcctctcaccctgtttcccagcgccaacctccggggc |
| | | aggcatccgggtggagagaggacttgtccctcgtgggtggtggttctttatagaaaaaatcg |
| | | aagcttagcagctcctcgtggcccgggtttggtaaaggctccagtgtggtggcctatgaaggacaatcctggcacgactac |
| | | tgcttccactgcaaaaaatgctccgtgaatctggccaacaagcgctttgttttccacc |
| 248 | 210299 | gcataggagataaaacccccactgagatgctctcatgcctcagctggacccaccgtgtagacacacgacatgcaagagttgcag |
| | _s_at | cggctgatccaactcactgctcaccctctctgtgagcagaaagaaccctactgacatgcatggttaacttcctcatcag |
| | | aactctgcccttcccttctgttcttttgtgctttcaaataactaacacgaacttccagaaaattaacatttgaacttagctgta |
| | | attctaaactgacctttccccgtactaacgtttggtttccccgtgtggcatgcttttctgagccttcctactttaaagcat |
| | | ggaacatgcaggtgatttgggaagtgtagaaagacctgagaaaacgagcctgtttcagaggaacatcgtcacaacgaatacttc |
| | | tggaagcttaacaaaactaaccctgctgtcctt |
| 249 | 210302 | atttcgttttgcttttggttgcctgaatgttgtcaccaagtgaaaaaattatttaactatatgtaaaatttctcttttaaaaaa |
| | _s_at | aagttttactgatgttaaacgttctcagtgccaatgtcagactgtgctcctccctctcctgaacctctaccctcaccctga |
| | | gctgtcttgttgaaaacagt |
| 250 | 210495 | agccctttgatctggaggaagttctccagcttcagctcaactcacagcttctccaagcatcaccctgggagtttcctgagggtttt |
| | _x_at | ctcataaatgagggctgcacattgcctgttctgcttcgaagtattcaataccgctcagtattttaaatg |
| | | aagtgattctaagatttggtttgggatcaataggaaagcatatgcagccaaccaagatgcaa |
| | | atgttttgaaatgatatgaccaaaattttaagtaggaaagtcacccaaacacttctgctttcacttaagtgtctggcccgcaat |
| | | actgtaggaacaagcatgatcttgttactgtgatattttaaatatccacagtactcactttttccaaatgatcctagtaatt |
| | | gcctagaaatatctttctcttacctgttatttatcaatttttcccagtatttttatacggaaaaaattgtattgaaaacactta |
| | | gtatgcagttgataagaggaatttggtataattatggtgggtgattattttttatactgtatgtgccaaagctttactactgtg |
| 251 | 210517 | gtgccatagtgcaggcttggggagctttaagcctcagttatataacccacgaaaaacagagcctcctagatgtaacattcctgatc |
| | _s_at | aaggtacaattctttaaaattcactaatgattgaggtccatatttagtggtactctgaaattggtcactttcctattacacgg |
| | | agtgtgctaaaactaaaaagcattttgaactcatacagaatgttctattgtcattgggaaatttttctttctaacccagtggagg |
| | | ttagaaagaagttatattctggtagcaaattaactttacatcctttttcctacttgttatggttgtttggaccgataagt |
| | | gtgcttaatcctgaggcaaagtagtgaatatgattatatgttatgaagaaaagaattgttgtaagtttttgattctactcttata |
| | | tgctggactgcattcacacatggcatgaaataagtcaggttctttacaaatggtattttgatagatactggattgtgtttgtgcca |
| | | tatttgtgccatt |
| 252 | 210524 | gtcaggactgttatggccctgttgtgctgagttagttggaacagaattcaagctccctgcactagtccacctgccccactgcttc |
| | _x_at | ttcgcttctctcttggaaagtccagtctctcctcggcttgcaatggaccccaactgctcctgcgccgctggtgtctcctgcacctg |
| | | cgctggttcctgcaagtgcaaagagtgcaaatgcacctcctgcaagaagagctgctgctcctgctgccccgtgggctgtagca |
| | | agtgtgcccagggctgtgtttgcaaaggggcgtcagagaagtgcagctgctgcgactgatgccaggacaacctttacccagat |
| | | gtaaacagagagacatgtacaaacctggattttttttttataccaccttgacccatttgctacattccttttcctgtgaaa |
| 253 | 210735 | caggaaggaccttgctttggaccctacacacttcggctctctggacacttgcgacacctcaaggtgttctctgtagctcaatct |
| | _s_at | gcaaacatgccaggcctcagggatcctctgctgggtgcctccttgccttgggaccatggccaccccagagccatccgatcgat |
| | | ggatgggatgcactctcagaccaagcagcaggaattcaaagctgcttgctgtaactgtgtgagattgtgaagtggtctgaattc |
| | | tggaatcacaaaccaagccatgctggtgggccattaatggttggaaaacactttcatccggggctttgccagagcgtgctttcaa |
| | | gtgtcctggaaagtctgctgcttctccaagctttcagacaagaatgtgcactctctgcttaggttttgcttgggaaactcaact |
| | | tctttcctctggagacggggcatctccctctgatttccttctgctatgacaaaacctttaatctgcaccttacaactcggggacaa |
| 254 | 210764 | ttaccaatgacaaccctgagtgccgccttgtgaaagaaacccggatttgtgaggtgcggccttgtggacagccagtgtacagca |
| | s_at | gcctgaaaaagggcaagaaatgcagcaagaccaagaaatcccccgaaccagtcaggtttacttacgctggatgtttgagtgtga |
| | | agaaataccggcccaagtactgcggttcctgcgtggacggccgatgctgcacgccccagctgaccaggacatgtgaagatgcg |
| | | gttccgctgcgaagatggggagacattttccaagaacgtcatgatgatccagtcctcaaatgcaactacaactgcccgcatgc |
| | | caatgaagcagcgtttcccttctacaggctgttcaatgacattcacaaatttagggactaaatgctacctgggtttccaggg |
| | | cacacctagacaaacaagggagaagagtgtcagaatcagaatcatggagaaaatgggcgggggtggtgtgggtgatgggactc |
| | | attgtagaaaggaagccttgctcattcttgaggagcattaaggtatttcgaaactgccaagggtgc |
| 255 | 210809 | aaattgtggagttagcctcctgtggagttagcctcctgtggtaaaggaattgaagaaaatataacaccttacaccctttttcatc |
| | _s_at | ttgacattaaaagttctggctaactttggaatccattagagaaaaatccttgtcaccagattcattacaattcaaatcgaagag |
| | | ttgtgaactgttatcccattgaaaagaccgagccttgtatgtatgttatggatacataaaatgcacgcaagccattatctctcca |
| | | tgggaagctaagttataaaaataggtgcttggtgtacaaaactttttatatcaaaaggctttgcacatttctatatgagt |
| | | gggtttactggtaaattatgttattttttacaactaattttgtactctcagaatgtttgtcatatgcttcttgcaatgc |
| 256 | 210946 | aaggcaagaggatgcatctttcttcctggtgtacaagcctttaaagacttctgctgctgatatgcctcttggatgcacactttgt |
| | _at | gtgtacatagttacctttaactcagtggttatctaatagctctaaactcattaaaaaaactccaagccttccaccaaaacagtgcc |
| | | ccacctgtatacatttttattaaaaaaatgtaatgcttatgtataaacatgtatgtaatatgctttctatgaatgatgtttg |
| 257 | 210982 | gaaggagacggtctggcggcttgaagaatttggacgatttgccagctttgaggctcaaggtgcattggccaacatagctgtggac |
| | _s_at | aaagccaacttggaaatcatgacaaagcgctccaactatactccgatcaccaatgacaagttcaccccaccagtggtcaatgt |
| | | cacgtggcttcgaaatggaaaacctgtcaccacaggagtgtcagagacagtcttcctgcccagggaagaccaccttttccgc |
| | | aagttccactatctccccttcctgccctcaactgaggacgtttacgactgcagggtggagcactggggcttggatga |
| | | gcctcttctcaagcactgggagtttgatgctccaagccctctcccagagactacagagaacgtggtgtgtgccctgggcctgac |
| | | tgtgggtctggtgggcatcattattgggaccatc |
| 258 | 211161 | aatattggatatcaactgcttgtaaaggtgctcctcctttttcttgtcattgctggtcaagattactaatatttgggaaggctt |
| | _s_at | taaagacgcatgttatggtgctaatgtactttcacttttaaactctagatcagaattgttgacttgcattca |
| | | gaacataaatgcacaaaatctgtacatgtctcccatcagaaagattcattggcatgccacaggggat |
| | | tctcctccttcatcctgtaaaggtcaacaataaaaaccaaattatggggctgcttttgtcacactagcatagagaatgtgtt |
| | | gaaatttaactttgtaagcttgtatgtggttgttgatcttttttttccttacagacacccataat |
| 259 | 211372 | atctcatacccgcaaattttaaccttgtcaacctctggggtattagtatgccctgacctgagtgaattcacccgtgacaaaac |
| | _s_at | tgacgtgaagattcaatggtacaaggattctcttcttttggataaagacaatgagaaatttctaagtgtgagggggaccac |
| | | tcacttactcgtacacgatgtggccctggaagatgctggctattaccgctgtgtcctgacatttgcccatgaaggcca |
| | | gcaatacaacatcactaggagtattgagctacgcatcaagaaaaaaaaagaagagaccattcctgtgatcatttccccc |
| | | ctcaagaccatatcagcttctctggggtcaagactgacaatcccgtgtaaggtgtttctgggaaccggcacacccttaacca |
| | | ccatgctgtggtggacggccaatgacacccacatagagagcgccta |
| 260 | 211538 | agctcgaaagagtttgcaaccccatcatcagcaaactttaccaaggtggtcctggcggcggcagcggcggcggcggttcagg |
| | _s_at | agcctccgggggacccaccatcgaagaagtggactaagcttgcactcaagtcagcgtaaacctcttt |
| | | gcctttctctctctctctttttttttgtttgtttctttgaaatgtccttgtgccaagtacgagatctat |
| | | tgttggaagtctttggtatatgcaaatgaaaggagaggtgcaacaacttagtttaattataaaagttccaaagtttgtttt |
| | | ttaaaaacattattcgaggtttctctttaatgcattttgcgtgtttgctgacttgagcatttttgattagt |
| | | tcgtgcatggagatttgtttgagatgagaaaccttaagtttgcacacctgttctgtagaagcttg |
| 261 | 211548 | gctttttttgttgtcagctatcttaagaatcattaaatacacctgctttgggtaaaactctttgcaagcagtaattaacacta |
| | _s_at | gtaacagtgaaagcacaagatttccaaatcagtcgttttctcaaaaaaatatcgtataagtgactcatcc |
| | | tgtctgctaactccagacctcccagcttgaagccaaatctttccatgtgagattgatatggatttccta |
| | | gaagtactggaatgttgtcatatcttgccctattttaattctgctatagaaaacaattgccttcacttttaaggagtaattt |
| | | gaatattaataactctggtctagattttcatataatgtattaaagacaaagtagtgaacatcaatgaacatctgatag |
| | | agataaactgtaatcaggcataagcttgtttgtatgttctggcagtgactaatcagtaaatg |
| | | atgtcggtttgcccagtatcacttatcttctg |
| 262 | 211549 | cgtgaacggcaaagtggcgctggtgaccggcgcggctcagggcataggcagagcctttgcagaggcgctgctgcttaagg |
| | _s_at | gcgccaaggtagcgctggtggattggaatcttgaagcaggtgtacagtgtaaagctgccctggatgagca |
| | | atttgaacctcagaagactctgttcatccagtgcgatgtggctgaccagcaacaactgagagacactt |
| | | ttagaaaagttgtagaccactttggaagactggacattaggtcaataatgctggagtgaataatgagaaaaactgggaaa |
| | | aaactctgcaaattaatttggtttctgttatcagtggaacctatcttggtttggattacatgagtaagca |
| | | aaatggaggtgaaggcggcatcattatcaatatgtcatctttagcagcccacc |
| | | attgattgccaatggattgataacactcattgaagatgatgctttaaatggtgctattatgaagatcacaacttctaag |
| | | ggaattcattttcaagactatgatacaactccatttcaag |
| 263 | 211596 | gagtattacattggccttgggggacagaaaggaggaagttctgacttttcagggctaccttatttctactaaggacccagag |
| | _s_at | caggcctgtccatgccattccttcgcacagatgaaactgagctgggactggaaaggacagcccttgacctgggtt |
| | | ctgggtataatttgcacttttgagactggtagctaaccatcttatgagtgccaatgtgtcatttagtaaaacttaaat |
| | | agaaacaaggtccttcaaatgttcctttggccaaaagctgaagggagttactgagaaaatagttaacaattactgtcaggt |
| | | gtcatcactgttcaaaaggtaagcacatttagaattttgttcttgacagttaactgactaa |
| | | tcttacttccacaaaatatgtgaatttgctgcttctgagaggca |
| 264 | 211637 | ggactggtgaagccttcggagaccctgtccctcacctgcagtgtctctggtggctccatcagtagttactactggagctgga |
| | _x_at | tccggcagcccccagggaagggactggagtggattgggtatatcaattacagtgggagcaccaact |
| | | ataacccctccctcaagagtcgagtcaccatatcagtagacacgtccaagaaccagttctcc |
| | | ctgaagctgagctctgtgaccgctgcggacacggccgtgtattactgtgcgagaacgatttcggggggtcg |
| 265 | 211643 | aatagtgatgacgcagcttccagccaccctgtctgtgtctccaggggaaagagccaccctctcctgcagggccagtcagagt |
| | _x_at | gttcgcagcaacttagcctggtaccagcaaaaacctggccaggctcccaggctcctcatctacggtgcatcc |
| | | accagggccaatggtatcccagccaggttcagtggcagtgggtctgggacagagttcactctc |
| | | accatcagcggcctgcagtctgacgattttgcagtttattactgtcaacactataataactggcctccgtggacgt |
| 266 | 211644 | aattgtgttgacgcagtctccaggcaccctgtctttgtctccaggggaaagagccaccctctcctgcagggccagtcaga |
| | _x_at | gtattagcagcagtttcttagcctggtaccagcagaaagttggccaggctcccaggctcctcat |
| | | ctatggtgcatccagcagggccactggcatcccagacaggttcagtggcagtgggtctgggacagac |
| | | ttcactctcaccatcagcagactggagcctgaagattttgcagtgtattactgtcagcaatatggtagctcgc |
| 267 | 211645 | tcctccctgtctgcatctgtaggagacagagtcaccatcacttgccgggcaagtcagggcattagaaatgatttagcctggta |
| | _x_at | tcagcagaaaccagggaaagcccctaagcgcctgatctatgctgcatccagtttgcaaagtgggg |
| | | tcccatcaaggttcagcggcagtggatctgggacagaattcactctcacaatcagcagcctg |
| | | cagcctgaagattttgcaacttattactgtctacagcataatagttacccttg |
| 268 | 211671 | gcagttgagtcgtcatcacttttcagtgatgggagagtagatggtgaaatttattagttaatatatcccagaaattagaaac |
| | _s_at | cttaatatgtggacgtaatctccacagtcaaagaaggatggcacctaaaccaccagtgcccaaagtctgtg |
| | | tgatgaactttctcttcatactttttttcacagttggctggatgaaattttctagactttctgttggtg |
| | | tatcccccccctgtatagttaggatagcatttttgatttatgcatggaaacctgaaaaaaagtttacaagtgtatatcaga |
| | | aaagggaagttgtgccttttatagctattactgtctggttttaacaatttcctttatatttagtgaactacgct |
| | | tgctcattattcttacataattattattcaagttattgtacagctgtttaagatgggcagctagttcgtagctt |
| | | tccca |
| 269 | 211696 | tgaggagaagtctgccgttactgccctgtggggcaaggtgaacgtggatgaagttggtggtgaggccctgggcaggctgctgg |
| | _x_at | tggtctacccttggacccagaggttctttgagtcctttggggatctgtccactcctgatgctgttatgggcaaccctaag |
| | | gtgaaggctcatggcaagaaagtgctcggtgcctttagtgatggcctggctcacctggacaacctcaagggcacctttg |
| | | ccacactgagtgagctgcactgtgacaagctgcacgtggatcctgagaacttcaggctcctgggcaacgtgctggtctg |
| | | tgtgccggcccatcactttggcaaagaattcacccaaccagtgcaggctgcctatcagaaagtggtggctgcgtggctaatg |
| | | ccctggcccacaagtatcactaagctcgctttcttgctgtccaatttctattaaaggttcctttgttccctaagtccaactac |
| | | taaactg |
| 270 | 211699 | agagaacccaccatggtgctgtctcctgacgacaagaccaacgtcaaggccgcctggggtaaggtcggcgcgcacgctggcgagta |
| | _x_at | tggtgcggaggccctggagaggatgttcctgtccttccccaccaccaagacctacttcccgcactt |
| | | cgacctgagccacggctctgcccaggttaagggccacggcaagaaggtggcc |
| | | gacgcgctgaccaacgccgtggcgcacgtggacgacatgcccaacgcgctgtccgccctgagcgacctgcacgcgcacaagcttc |
| | | gggtggacccggtcaacttcaagctcctaagccactgcctgctggtgaccctggccgcccacct |
| | | ccccgccgagttcacccctgcggtgcacgcctccctggacaagttcctggcttctgtg |
| | | agcaccgtgctgacctccaaataccgttaagctg |
| 271 | 211719 | gggagtttcctgagggttttctcataaatgagggctgcacattgcctgttctgcttcgaagtattcaataccgctcagtatttta |
| | _x_at | aatgaagtgattctaagatttggtttgggatcaataggaaagcatatgcagccaaccaagatgcaaatgttt |
| | | tgaaatgatatgaccaaaattttaagtaggaaagtcacccaaacacttctgctttcacttaa |
| | | gtgtctggcccgcaatactgtaggaacaagcatgatcttgttactgtgatattttaaatatccacagtactcactttttccaaa |
| | | tgatcctagtaattgcctagaaatatctttctcttacctgttatttatcaatttttcccagtatttttata |
| | | cggaaaaaattgtattgaaaacacttagtatgcagttgataagaggaatttggtataattatggtgggtgatt |
| | | attttttatactgtatgtgccaaagctttactactgtg |
| 272 | 211745 | tcctgccgacaagaccaacgtcaaggccgcctggggtaaggtcggcgcgcacgctggcgagtatggtgcggaggccctggaga |
| | _x_at | ggatgttcctgtccttccccaccaccaagacctacttcccgcacttcgacctgagccacggctctgcccaggtta |
| | | agggccacggcaagaaggtggccgacgcgctgaccaacgccgtggc |
| | | gcacgtggacgacatgcccaacgcgctgtccgccctgagcgacctgcacgcgcacaagcttcgggtggacccggtcaacttc |
| | | aagctcctaagccactgcctgctggtgaccctggccgcccacctccccgccgagttcacccctgcgg |
| | | tgcacgcctccctggacaagttcctggcttctgtgagcaccgtgctgacctccaaata |
| | | ccgttaagctggagcctcggtggc |
| 273 | 211798 | tacgcagactccgtgaagggccgattcaccatctccagagacaattccaagaacacgctgtatcttcaaatgaacagcctga |
| | _x_at | gagccgaggacacggccgtattactgtgcaagagagacttgtgtgattggggccaaggtaccctggtcacc |
| | | gtgtcgagaggtggcggtggctcgggcggtggtgggtcgggtggcggcggatctt |
| | | ctgagctgactcaggaccctgctgtgtctgtggccttgggacagacagtcaggatcacatgccaaggagacagcctcaga |
| | | agctattatgcaagctggtaccagcagaagccaggacaggcccctgtacttgtcatctatggtaaaaacaacc |
| | | ggccctcagggatcccagaccgattctctggctccagctcaggaaacacagcttccttgacc |
| | | atcactggggctcaggcggaagatgaggctgactattactgtaactcccgggacagcagtggtaaccatgtggtattcgg |
| | | cggagggaccaagctgaccgtcctaggtgcggccgcagaacaaaaactcatctcagaagaggatctgaatggggccgcat |
| 274 | 211813 | ttgcacaagtttcctgggctggaccgtttcaacagagaggcttatttgactttatgctagaagatgaggcttctgggataggcc |
| | _x_at | cagaagttcctgatgaccgcgacttcgagccctccctaggcccagtgtgccccttccgctgtcaatgccatcttcga |
| | | gtggtccagtgttctgatttgggtctggacaaagtgccaaaggatcttccccctga |
| | | cacaactctgctagacctgcaaaacaacaaaataaccgaaatcaaagatggagactttaagaacctgaagaaccttcacgt |
| | | tgtctaccttcataacaacaatatctctgtagttggatcaagtgacttctgcccacctggacacaa |
| | | caccaaaaaggcttcttattcgggtgtgagtcttttcagcaacccggtccagtactgggagatac |
| | | agccatccaccttcagatgtgtctacgtgcgctctgccattcaactcggaaacta |
| 275 | 211848 | tgtccatacagagtgtgcattccctggcaggggctcctgctcacagcctcgcttttaaccttctggaacctgccaaacagtgc |
| | _s_at | ccagaccaatattgatggtgtgccgttcaatgtcgcagaagggaaggaggtccttctagtagtccataatgag |
| | | tcccagaatctttatggctacaactggtacaaagggcaaagggtgcatgccaacta |
| | | tcgaattataggatatgtaaaaaatataagtcaagaaaatgccccagggcccgcacacaacggtcgagagacaatataccc |
| | | caatggaaccctgctgatccagaacgtcacccacaatgacgcaggaatctataccctacacgttataa |
| | | aagaaaatcttgtgaatgaagaagtaaccagacaattctacgtattctatgagtcagtacaagc |
| | | aagttcacctgacctctcagctgggaccgctgtcagcatcatgattggagtactggctgggatggctctgatatagc |
| 276 | 211889 | tgcacagtactcctggcttatcaatggaacattccagcaaagcacacaagagctctttatccctaacatcactgtgaataat |
| | _x_at | agtggatcctatacctgccacgccaataactcagtcactggctgcaacaggaccacagtcaagacgatcatag |
| | | tcactgagagacagaatctcaccatgttgcccgggctggactcgaactcctgggct |
| | | caagcaatcctcccatctgtttcccaaagtgctgagattacagataatgctctaccacaagaaaatggcctctcacctggg |
| | | gccattgctggcattgtgattggagtagtggccctggttgctctgatagcagtagccctggcatgtttt |
| | | ctgcatttcgggaagaccggcagggcaagcgaccagcgtgatctcacagagcacaaaccctcagtgtccaa |
| | | ccacactcaggaccactccaatgacccacctaacaagatgaatgaagttacttattctaccctgaactttgaagccca |
| 277 | 211896 | tcctccttcccttacggaattacatcttgatggcaacaaaatcagcagagttgatgcagctagcctgaaaggactgaataa |
| | _s_at | tttggctaagttgggattgagtttcaacagcatctctgctgttgacaatggctctctggccaacacgcctcatctgaggga |
| | | gcttcacttggacaacaacaagcttaccagagtacctggtgggctggcagagcataagtacatccaggttgtctaccttcat |
| 278 | 211959 | tcacaccgtagcccacatggatagcacagttgtcagacaagattccttcagattccgagttgcctaccggttgttttcgtt |
| | _at | gttgttgttgttgtttttctttttctttttttttttgaagacagcaataaccacagtacatattact |
| | | gtagttctctatagttttacatacattcataccataactctgttctctcc |
| 279 | 211964 | cactttccaatgccacagacaactcacattgttcaactcccnttctcggggtgggacagacgagacaacagcacacaggcagcc |
| | _at | agccgtggnnccagaggctcganggggctcaggggcntcanggcacccgtccccacacgn |
| | | agggccccgtngggtgggncctnggccctngcttctacngccnaatgnttatgnccag |
| | | ctccatgttctcccaaataccgttgatgtgaattattttaaaggcaaaacngtgctctttattttanaaaacactgataatca |
| | | cactgcggtaggtcattcttttgccacatccctatagaccactgggtttggcaaaactcaggcagaagtgg |
| | | aganccnttctagacatcantgtcagccttgctacttgaaggtacaccccatagggtcggag |
| | | gtgctgtcccc |
| 280 | 211985 | gaggcaaatggatctcgatatttcagatgggcttttgatgcactgttgccaaggaaggctttttctgattttttgacaaatga |
| | _s_at | atttttgcacactttcattggtgtctttcggcaacttacacacattgaaaat |
| 281 | 211990 | gaggacttaggagagatctgaactccagctgccctacaaactccntcncagcttttcttctcacttcatgtgaaaactactcc |
| | _at | agtggctgactgaattgctgacccttcaagctctgtccttatccattacctcaaagcagtcattccttagtaaagtt |
| | | tccaacaaatagaaattaatgacactttggtagcactaatatggagattatcctttcatt |
| | | gagccttttatcctctgttctcctttgaagaacccctcac |
| 282 | 211991 | gcactgggaggcccaagagccaatccagatgcctgagacaacggagactgtgctctgtgccctgggcctggtgctgggccta |
| | _s_at | gtnggcatcatcgtgggcaccgtcctcatcataaagtctctgcgttctg |
| 283 | 212077 | tgcagaagtcaaaccctcatgacaaagtaggcacaagtctacaataagctaaatcagaatttacaaatacaagtgtcccag |
| | _at | gtagcattgactcccgtcattggagtgaaatggatcaaagtttgaattaaggcctatggtaaggtaacattgct |
| | | ttgttgtacttttgaacaagagctcctcctgatcactattacatatttttctagaaaatct |
| | | aaagttcagaagagaatgtatcactgctgacttttattccaatatttggatggagtaagttttagggtagaattgttcagt |
| | | ttggatttaatcttttgaaaagtaaattccttgtttactggtttgactataattctctgttatctttacgaggta |
| | | aaactgcaagctgactagcatgttctgtgaatctgccattcctaaaaattttataaacacttgatacttttc |
| | | actgataatggatcgctcca |
| 284 | 212091 | tcacagcgggcaggacggaccccgcccacgacgtgcgggtggcggtggtgcagtacagcggcacgggccagcagcgcccag |
| | _s_at | agcgggcgtcgctgcagttcctgcagaactacacggccctggccagtgccgtcgatgccatg |
| | | gactttatcaacgacgccaccgacgtcaacgatgccctgggctatgtgacccgcttctac |
| | | cgcgaggcctcgtccggcgctgccaagaagaggctgctgctcttctcagatggcaactcgcagggcgccacgcccgctgc |
| | | catcgagaaggccgtgcaggnagnccagcgggcaggcatcgagatcttcgtggtggtcgtgggccgcca |
| | | ggtgaatgagccccacatccgcgtcctggtcaccggcaagacggccgagtacgacgtggcctacggcgagagc |
| | | cacctgttccgtgtccccagctaccaggccctgctccgcggtgtcttccaccagacagtctccaggaaggt |
| 285 | 212097 | gaatttcacctgtaaacctgagtcgtacagaaagctgcctggtatatccaaaangctttttattcctcctgctcatattgtgatt |
| | _at | ctgccntttggggnacttttncttnaaaccttcagttatgatttttttttncatacacttattggnaactctgcttgatt |
| | | tttgcctcttccagtcttcctgacactttaattaccaacctgttacctactttgactttttgcattta |
| | | aaacagacactggcatggatatagttttacttttaaactgtgtacataactgaaaatgtgctatactgcatacttataaatgta |
| | | aagatatttttatctttatatgaagaaaatcacttaggaaatggctttgtgattcaatctgtaaactgtgtat |
| | | tccaagacatgtctgttctacatagatgcttagtccctcatgcaaatcaattactggtccaaaagattgct |
| | | gaaattttatatgcttactgatatattttacaatttatatcatgcatgtcctgtaaaggttacaagcctgcacaataa |
| 286 | 212136 | tcttccccttgagcttcagagaggagagttggcatggttaaatctgaatggttacctcactgctgaaaacccagaggggcgt |
| | _at | ggcacactcgcttgtgtggaaaagcctctaaatgcatcccttcctttctttcctgcttcctttgccttacaattgaagc |
| | | agcccgtggtaccatcacagtatgcagagacttcctcacctttcatatctagggacc |
| | | acccccgatgcattggtgagggtgggcacttataaatgcctgctattgttaagccattccagcctcttcctctgaataga |
| | | ccagacgccctttcacttagttcagtgccagtcatttgccttcccaaccctgctgttaggcctgctgttcccttt |
| | | gctcttgattaggagagatggaaggagatgagctcccataactgaattggcctttggttcatgttt |
| | | tctccccatatgtatatatgccatatgtgaatatgccatatatatgtgccaacaaatctatctacgttgttcttttcaaa |
| | | ttagcacgcagataggaattttgagtttcttcttctt |
| 287 | 212158 | tatgtaaactttaacttccactttgtataaatttttaagtgtcagactatccattttacacttgctttatttttcattacct |
| | _at | gtagctttgggcagatttgcaacagcaaattaatgtgtaaaattggattattactacaaaaccgtttagtcatatctatc |
| | | taatcagatcttcttttgggaggatttgatgtaagttactgacaagcctcagcaaacccaaagatg |
| | | ttaacagtattttaagaagttgctgcagattcctttggccactgtatttgttaatttcttgcaatttgaaggtacgagta |
| | | gaggtttaaagaaaaatcagtttttgttcttaaa |
| | | aatgcatttaagttgtaaacgtctttttaagcctttgaagtgcctctgattctatgtaacttgttgcagactggtg |
| 288 | 212185 | cgccggctcctgcaaatgcaaagagtgcaaatgcacctcctgcaagaaaagctgctgctcctgctgccctgtgggctgtg |
| | _x_at | ccaagtgtgcccagggctgcatctgcaaaggggcgtcggacaagtgcagctgctgcgcctgatg |
| | | ctgggacagccccgctcccagatgtaaagaacgcgacttccacaaacctggattt |
| 289 | 212192 | caactgtttttgcgactttataggcaggtaaattttgctattactattgaatacaaatgacaattcatttatgaccactcaaa |
| | _at | cagcgttagtaaccatttagtgacaaaggattaaaacatccatctggatgttaattttgaagatgtaaattatatgttgtt |
| | | taaatttttccaggcatctgaaaaccttatctgctagacaatgtaagattcacacagag |
| | | ttatctgggattctgattttttaaatagtacatatcattaaaccattttctctaaatgtaagaagagcagaaaaaatctta |
| | | taagattatcagatttttctaatgacacagaaatgtaagaaaaaaatccctttatattgaaaaaagatgcagtcaaa |
| | | gtcttttcagacatgcccaaactttgagaatttcttcaaccatctaatgctataaagatttttg |
| | | ttcttcctgttcacaaccagttgtataacagaaatactagctactgttttccttc |
| 290 | 212195 | gatgggtcgtgtgatgagatgcatttaaggccgatagtgatagatgttttttttatttcttgaacacaggctttgtctgaatg |
| | _at | atgttcttttatctcttgaacacaagctttgaatgataactacaggttttaagtgctgttacattaataccataat |
| | | gtgatgtgttagaaacaaagggatatttcaaaggtagatatttgaaaattctctagtctcaatatgt |
| | | atgtgtattgaatatactctaaaaataaatgtgcaatttgctagtaggacaatgcagtgactgactagcattaggtatgtt |
| | | tcttttatatcctagctatgtcccactttcttctaagtgcaatcctttcatgttcacttgctgttttaccccatcta |
| | | ctctaacttcatttggaaggcttgtctagagtatagcatgtatttttacctttgcagtgaattgcatgtgctaattgtaac |
| | | cacagctatttttatgttgacataactccaaatgttatattaaatgttctattatatattagctctaatcccttaagtaaa |
| 291 | 212224 | acagtgttctctaatgttacagatgagatgcgcattgccaaagaggagatttttggaccagtgcagcaaatcatgaagtttaa |
| | _at | atctttagatgacgtgatcaaaagagcaaacaatactttctatggcttatcagcaggagtgtttaccaaag |
| | | acattgataaagccataacaatctcctctgctctgcaggcaggaacagtgtgggtgaatt |
| | | gctatggcgtggtaagtgcccagtgcccctttggntgggattcaagatgtctggaaatggaagagaactgggagagtacggt |
| | | ttccatgaatatacagaggtcaaaacagtcacagtgaaaatctctcagaag |
| | | aactcataaagaaaatacaagagtggagagaagctcttcaatagctaagcatctccttacagtcactaatat |
| 292 | 212230 | caaggtcccactctaggtgatagacagggaccccttctactgaacctttgaggaaaggaggaaggaagaaatgcgtttagat |
| | _at | cttggatgcagacctttcaaagggttaaatgtaaccatatggatcaaccacatgcacatccttactacag |
| | | aatccgtcctttcatttcaacttatagcaagctatgatttttatatataaatattatataaataatgtataa |
| | | aacattaaaagttaactatgtaagatattatttctgaaacaatttagctatatccactatgattataaactgtgtct |
| | | cgacctgtgttatttacattagctgcttaaaaaagcattgagttaatttttttaaatatcaactaaaatatcatagtt |
| | | ctgtggtagacattgttttataatgaaataactgcaactagagaaaactgtataaaaacattaaattgtcagtatttt |
| | | tgtaaggttccattttgtaaagagaataatattcaaagacttttgtagcatacaaagtgaaaacttgtatctgcgaaacta |
| 293 | 212233 | aagcagcctagggatgagcatttctttgaaagcaattaggttattcacctggtattaaaactatttactgttaaaaaatctgtg |
| | _at | acttcatgaagttgatttttaaaggcagcatcaaaaactgaaaaggnaagggaaaaaataggcagcttctct |
| | | gcacttgtttggagctccccaaaacaggagccatggagaagtggcatcaagaccgg |
| | | gctgccctttcgagaacaccctgtggcagttcagagacacgcttttcctacactgcatgcagcccctctttccagcactgga |
| | | aagaagtggtcttgagcccagctgagaagcacttcacactcctctctcttgttctgaatggtgt |
| | | ttgtgtcagtctgcagctgtgtatggtattatgtcttataatcctgcatcacttctatcctatccagtcatat |
| 294 | 212265 | gggtagatgggagtgtcgcttgtatgttatcgtacagctgacatgtatttttgtctattctttattatcttagtttcatgctatg |
| | _at | tatgtaccataaccaacctattgcctatgagaaacatgtaagataatgtatttacagccattgttacaagtttataatgta |
| | | tttttctatcttgttttatatgtatgttatataacattcaaaaagaatttttttcttgattgagaaaa |
| | | ggatacaaaatgcaaaatccacaattttgataactgaaaattgccaattgttttgcagtactttattatattgggtgtcttgtc |
| | | ttttttgggcttttagttagctaatgaatgaatacattagacacttttgggttttagttgggattttacat |
| | | agcttgcattttaattctttggttctttgctgtttctattaacccacagcattatt |
| 295 | 212288 | gaaggttgtcatgggctgttccttgggggtttttatcctgctcaccgtggagataagcctgcggcttgtctaaccagcgcagcgc |
| | _at | aaaggtctcaatgccttttggtaacatccgtcattgcagaagaaagtttacacgacgtcaaaaagtgacgttc |
| | | atgctaagtgtttttccagaaatattggtttcatgtttcttattggctctgcctcctgtgcttatatcatcca |
| | | aaaactattaaaaaggtccagaattctattttaacctgatgttgagcacctttaaaacgttcgtatgtgtgttgcactaattc |
| | | taaactttggaggcattttgctgtgtgaggccgatcgccactgtaaaggtcctagagttgcctgtttgtctctggagat |
| 296 | 212386 | gagatttaccatgtatcagtgcctggctttgttataaagctttgtttgtctagtgctcttttgctataaaatagactgtagtac |
| | _at | accctagtaggaaaaaaaaaaaactaaatttaaaaataaaaaatatatttggcttatttttcgcaggagcaatccttttatacc |
| | | atgaatatt |
| 297 | 212387 | gtttatacaactgtcagtccacccagtggtgcaactggttctgattcagtcttccgatncctttttatttttcactttttcctat |
| | _at | ttctgaattttttttttnatttgtgatcttgattttgatgaggggttggggagtggggagggagtcgaaccaagacttggagtta |
| | | agaggattttcatcttttgcatccaacaggcagaatatgatctgtgtccaaaagtgaa |
| | | cttgagtcaggaatgaatcaatttcagcataaacaagcacaaaaatttagtctgctggctgactggaagcaaaaaagtcaagat |
| | | ggaatatgatgaattccaacacaatggggcaccaaggcctttaggcctctattttattttgctttgg |
| | | ttttgtttgtttttctttagagacatgctctttctcatgggacttgaagtggactcatctttgtgcagtgctggtttt |
| | | gccatactcatttcaagtattatagacatatgtaatggtgaaaatatatgaactgtggcctttttcattcttgttacttgtg |
| 298 | 212397 | gaagtgactgttgtaccatggttgtgcacatgcttcagaatcctatggaagagaatattcctacttgcagtacatcaaagga |
| | _at | atggatggtggaccctactattcatgttttgagacataaatgttcactttaaagcaattgcataatagataaaa |
| | | acctgaactttcattggatttttgttaattttcctcattttgaattatgtgcactaccatagctacat |
| | | cagtttgatacagtattgaaaaattatcagttatattgctgtttatgatctatttgtagattaggattaaaatggatttaatcc |
| | | atttttaaggctgtgtgaatttttctaaacaagaaccatttgcaatatggatttcttagagattaaaccaatta |
| | | taacttattagcagtcgcgagcacatgttcatatagtcaatgtaaaaatacactaatgagtatttgg |
| | | taaatcccagtaggcttttaccattagca |
| 299 | 212414 | tccctcacttctcttaagccaagaagtttgctttccctagctgcagtgtagatggctcttgtttttgtttttttgttttaatca |
| | _s_at | tttggcattcacatgtggctgttaatatgtgcttgttttt |
| 300 | 212419 | tggtgggagggctctcaatcagcagggccccaggagggaagaagaagtggggcaaagcntggcctcgccgctcgggagctttgc |
| | _at | catctgagccacgcctcctccaggccatgctccttgaacttggaaatgtcaaccggagcccttacacca |
| | | gccctccagcatctaatagacttgaatctactctaaacgaatatttaatccaacc |
| | | tcactacattgtagctcagtccaacgactaaccctgaaatgggggtgttccagccttcagcgagatggccaagcggtcccctg |
| | | ggggctgtggcagcggncttatccttctctgttgccaaccttgccgtccgacctcctccgcccccatgcgg |
| | | tgaccccgtccgtgtctgtgtctgtccatacgtgtgagtccagctaaaaagacaaaaca |
| | | gaacccgtgggcccagctcggaaggtgcgtggagaaggctccgacgtctccgaagtgcagcccttgggatggcattccgttg |
| | | tgtgccttattcctggagaatctgtatacggctcgcctatagaa |
| 301 | 212464 | cttctccaagcatcaccctgggagtttcctgagggtttctcataaatgagggctgcacattgcctgttctgcttcgaagtat |
| | _s_at | tcaatanccgctcagtattttaaatgaagtgattctaagantttggtttgggatcaatanggaaagncatat |
| | | gnncagnnccaanccaagatgcaaatgttttgaaatgatatgaccaaaattttaagtaggaa |
| | | agtcancccaaacacttctgctttcacttaagtgtctggcccgcaatactgtaggaacaagcatgatcttgttactgtgat |
| | | attttaaatatccacagtactcactttttccaaatgatcctagtaattgcct |
| 302 | 212592 | gtatcaaaatcttccaattatcatgctcacctgaaagaggtatgctctcttaggaatacagtttctagcattaaacaaataa |
| | _at | acaaggggagaaaataaaactcaaggagtgaaaatcaggaggtgtaataaaatgttcctcgcattcc |
| | | cccccgcntttttttttttttttgactttgccttggagagccagagcttccgcattttctttactattcttttt |
| | | aaaaaaagtttcactgtgtagagaacatatatgcataaacataggtcaattatatgtctccattagaaaaataataattgg |
| | | aaaacatgttctagaactagttacaaaaataatttaaggtgaaatctctaatattt |
| | | ataaaagtagcaaaataaatgcataattaaaatatatttggacataacagactt |
| 303 | 212667 | caggaggacacagcattattctagtgctgtactgttccgtacggcagccactacccacatgtaactttttaagatttaaattta |
| | _at | aattagttaacattcaaaacgcagctccccaatcacactagcaacatttcaagtgcttgagagccat |
| | | gcatgattagtggttaccctattgaataggtcagaagtagaatcttttcatcatcacagaaagttct |
| | | attggacagtgctcttctagatcatcataagactacagagcacttttcaaagctcatgcatgttcatcatgttagtgtcgtatt |
| | | ttgagctggggtatgagactncccttagagatagagaaacagacccaagaaatgtgctcaattgcaatgggccacat |
| | | acctagatctccagatgtcatttcccctctcttattttaagttatg |
| 304 | 212671 | accaatgaggttcctgaggtcacagtgtttccaagtctcccgtgacactgggtcagcccaacaccctcatctgtcttgtggaca |
| | _s_at | acatctttcctcctgtggtcaacatcacntggctgagcaatgggcactcagtcacagaaggtgtttctg |
| | | agaccagcttcctctccaagagtgatcattccttcttcaagatcagttacctcaccttcctccc |
| | | ttctgntgatgagatttatgactgcaaggtggagcactggggcctggatgagcctcttctgaaacactgggagcctg |
| 305 | 212713 | cacatggttctagcctggacctggctgggctccatgagaatgagttgcctccaccctgtcccaacagctgacagccaggagcca |
| | _at | ctctcccagctgcaggcctttgtggtccatcttgtcctgcttcctcactgtggacccctgtctggg |
| | | ccaccctagtgtgctaagctgagcagtgcagtgtgaacagggcccatggtgtattctaggcca |
| | | cagcccagcactcctctgggctgctctcaaaccatgtcccatcttcagcatccctcccaccaacttactcccctgtggtgagt |
| | | accgtggaaccccagcccacctcactatcatactcagcttcccctgatggcccatcccagcccctgaagctn |
| | | tatgccaagaacacagctaccgcacaccaccctgaaacagccacagccaaggta |
| | | ggcatgcatatgaggtcttccccataccctctgggtgttgagaggtttagccacatgagggagcagaggacaatctctgca |
| | | gggctgggagtgggtagggactgaaggtctcaat |
| 306 | 212730 | tcagtgtctgggctcatgacttgtaaatggaagctgatgtgaacaggtaattaatattatgacccacttctatttactttggga |
| | _at | aatatcttggatcttaattatcatctgcaagtttcaagaagtattctgccaaaagtatttacaagtatggactcatgagctat |
| | | tgttggttgctaaatgtgaatcacgcgggagtgagtgtgcccttcacactgtgac |
| | | attgtgacattgtgacaagctccatgtcctttaaaatcagtcactctgcacacaagagaaatcaacttcgtggttggatggggc |
| | | cggaacacaaccagtctttttgtatttattgttactgagacaaaacagtactcactgagtgtttt |
| | | tcagtttcctactggtggttttgatattgtttgtttaagatgtatatttagaatgacatcatctaagaagctgat |
| | | tttgctaaactcctgttccc |
| 307 | 212741 | gaactcatgtgatttacccttttcaactttttggaaaacgatttaatttattctaattagattaaccctattaatctatgga |
| | _at | ttgggtatcaaaatgaatgccagtccagatgtgcctagacacgaaattggagctgaggactctcacgatat |
| | | gcaagttcatccaacgtgaagataccataagctttttctctgaaccagagaaatgaaagtcagttta |
| | | agaggctgatagatcttggccctgttaaggcatccacttcacagttctgaaggctgagtcagccccactccacagttaggc |
| | | caagaattagattttaaaacttcatctgtctgtcccagttaactgttaaataaggcctcatcctccactgaagag |
| | | tatggattgaaggattgtgaactatgtttagtgtgattgtgaacttggtgcctaatgttccat |
| | | gtctgaagtttgccccagtgctacacgttggagta |
| 308 | 212764 | gatgcaattggttctcctgcattgagatttgatttaacagtgttatgttaacatttatacttgccttggactgtagaacaga |
| | _at | acttaaatgggaatgtattagttttacaactacaatcaagtcattttacctttacccagtttttaatata |
| | | aaacttaaattttgaaattcactgtgtgactaatagcatgatgctctgcagttttattaagaaatnagcctaacc |
| | | atacaactctcatttccttagtaagccaaattaggattaacttctataaacagtgttgggaacaatgtttaacattttgtgc |
| | | caatttgttcctgtattcatgtatgtaagttacagatctgactcttcatttttaagttccttgttacatcatggtcatt |
| 309 | 212814 | cctatcctacttgcccatatgagcacggctccccatggccacatactcctgcaaagcttttatgctgcttcgcttttctctaaa |
| | _at | cagatctgatattgctgctcctgtggttttctcaaaattaactttgccgtggtttttaaaaaggaatca |
| | | aaatgcattgttgcattaagctttttcaataaaggaaaattacggaaggaaaataggcaacaccagca |
| | | aattatatgtggacaggttctaaactctatatatacatatatatatatatatctatatatctatatacgtaatcatctagttct |
| | | gtcatcttactgaaaggaataacacttctaaagatcaccatttctgagaagttcttggaaatctttatgtctaa |
| 310 | 212859 | caccgcgcagagctcagggggtggtgcgccaggcccttctgcggcgcacagcccagcccaggaacgcgggcggtgcggactcag |
| | _x_at | cgggccgggtgcaggcgcggagctgggcctctgcgcccggcccganctccgtctataaanagagcagccagt |
| | | tgcagggctcnantctgctttccaactgcctgactgcttgttcgtctca |
| | | ctggtgtgagctccagcatcccctttgctcgaaatggaccccaactgctcttgcgccactggntggctcctgcacgtgcgccgg |
| | | ctcctgcaagtgcaaagagtgcaaatgcacctcctgcaagaagagctgctgttcctgctnccccgtggnctgt |
| | | gccaagtgtgcccagggctgcgtctgcaaaggggcatcggagaagtgcagctgctgtgcctgatgtgggaacagc |
| | | tcttctcccagatgtaaatagaacaacctgcacaacctggnatttttttaaaaatacaacactgagccatttgctgcatttc |
| 311 | 212956 | tactgatgcttctgatactggatgtttagcttcttactgcaaaaacataagtaaaacagtcaactttaccatttccgtattctc |
| | _at | catagattgaagaaatttataccacatatcgcatatgaccatctttccatcaaatcaatgtagagataatgtaaactgaa |
| | | aaaaaatctgcaagataatgtaactgaatgttttaaaaacagaacttgtcactttat |
| | | ataaaagaatagtatgctctatttcctgaatggatgtggaaatgaaagctagcgcacctgcactttgaattcttgcttctttt |
| | | ttattactgttatgattttgctttttacagatgttggacgattttttcttctgattgttgaattcataatcatggtctc |
| | | atttcctttgcttctttggaatatttctttcaacacattcctttattttattatacattgtgtcctttttttagctattg |
| | | ctgct |
| 312 | 213068 | gacaggagggtgtccacatatgttaacatcagttggatctcctatagaagtttctgctgctctctttccttctccctgagct |
| | _at | ggtaactgcaatgccaacttcctgggcctttctgactagtatcacacttctaataaaatccacaa |
| 313 | 213071 | gctccacatgagccatgcatgcttagcaatccaagtgcagagctctttgctccaggagtgaggagactgggaggtgaaatggg |
| | _at | gaaatggaagggtttggaggcagagctgaaaacagggttggaaggatttcctgaattagaagacaaacgt |
| | | tagcatacccagtaaggaaaannngnnnaggggccanggggaacccgtg |
| | | aggatcactctcaaatgagattaaaaacaaggaagcagagaatggtcagagaatgggattcagattgggaacttgtggggatg |
| | | agagtgaccaggttgaactgggaagtggaaaaaggagtttgagtcactggcacctagaagcctgcccacga |
| | | ttcctaggaaggctggcagacaccctggaaccctggggagctactggca |
| | | aactctcctggattggncctnatnnttttggtgggaaaggctgccctggggatcaactttccttctgtgtgtggctcaggag |
| | | ttcttctgcagagatggcgctatctttcctcctcctgtgatgtcctgctcccaaccatttgtactcttcatta |
| 314 | 213317 | aagtgtgcataatttcatttaacgttaaagaaatagatccaattcctttcttgcaaccaaaaataaataaaatacgttgcctcaa |
| | _at | tataaggtttgggctattctgtgtttctatagaagcaatctgtttttggtaaaatgtactataaggatccagtcatctga |
| | | agtattttatgtagagttagagatttcacaatattgactatacatatatttaaaatataa |
| | | attatccagctgatgtttgaatttgtcttactttcctggccacctcgttgtcctattttataagctggggagttaactagcttaa |
| | | caaaagatgcttagcttttgtaaaagaacaagtgtttcattttacaaagacactccaaatgatagttacttgattttctcga |
| | | gacctttaactatggtgatgaataacaggacttgctttcaagccttaataaatgtaaa |
| | | atgccttttaatgaagatacagctgagtgttttcctcatgaatctgaaccaattaccaatttgtgttccagtcttgatt |
| 315 | 213428 | gaaaccaaggtcaggaggccgttgcagacataaatctcggcgactcggccccgtctcctgagggtcctgctggtgaccggcctg |
| | _s_at | gaccttggccctacagccctggaggccgctgctgaccagcactgaccccgacctcagagagtactcgc |
| | | aggggcgctggctgcactcaagaccctcgagattaacggtgctaaccccgtctg |
| | | ctcctccctcccgcagagactggggcctggactggacatgagagccccttggtgccacagagggctgtgtcttactagaaaca |
| | | acgcaaacctctccttcctcagaatagtgatgtgttcgacgttttatcaaaggccccctttctatgtt |
| | | catgttagttttgctccttctgtgtttttttctgaaccatatccatgttgctgacttttc |
| 316 | 213451 | cacaggcaggtgactactccatgcgcgtggacctgcgggctggggacgaggctgtgttcgcccagtacgactccttccacgta |
| | _x_at | gactcggctgcggagtactaccgcctccacttggagggctaccacggcaccgcaggggactccatgagctaccaca |
| | | gcggcagtgtcttctctgcccgtgatcgggaccccaacagcttgctcat |
| | | ctcctgcgctgtctcctaccgaggggcctggtggtacaggaactgccactacgccaacctcaacgggctctacgggagcaca |
| | | gtggaccatcagggagtgagctggtaccactggaagggcttcgagttctcggtgcccttcacggaaatga |
| | | agctgagaccaagaaactttcgctccccagcggggggaggctgagctgctgccc |
| | | acctctctcgcaccccagtatgactgccgagcactgaggggtcgccccgagagaagagccagggtccttcaccacccagcc |
| | | gctggaggaagccttctctgccagcgatctcgcagcactgtgtttacag |
| 317 | 213509 | ccaatggcgagggtctgccacactggccgctgttcgaccaggaggagcaatacctgcagctgaacctacagcctgcggtgg |
| | _x_at | gccgggctctgaaggcccacaggctccagttctggaagaaggcgctgccccaaaagatccaggagctcgag |
| | | gagcctgaagagagacacacagagctgtagctccctgtgccggggagga |
| | | gggggtgggttcgctgacaggcgagggtcagcctgctgtgcccacacacacccactaaggagaaagaagttgattccttc |
| | | attcacttcgccattcattcatacttccgtccatccattcagaaagcatttattaagaatttactcaggcatg |
| | | atggcccatacttgtaatcccagctattgggaaggatgagatgggaggatggcttgaggcc |
| | | agaggtttgagaccgaccagcc |
| 318 | 213624 | gggagagtccatctggaagctggagtatatcctgacccagacctacgacattgaagatttgcagccggaaagtttatatg |
| | _at | gattagctaaacaatttacaatcctagacagtaagcagtttataaaatactacaattacttctttgtgagt |
| | | tatgacagcagtgtaacatgtgataagacatgtaaggcctttcagatttgtgcaattatgaatctt |
| | | gataatatttcctatgcagattgcctcaaacagctttatataaagcacaattactagtatttcacagtttttgctaatag |
| | | aaaatgctgattctgattctgagatcaatttgtgggaattttacataaatctttgttaattactgagtgggcaagtagac |
| | | ttcctgtctttgctttctttttttttttctttttgatgccttaatgtagatatctttatcattctgaattgtatta |
| | | tatatttaaantgctcattaatagaatgatggatgtaaattggatgtaaatattcagtttatataattatatctaatttg |
| | | tacccttgttgaaattgtcattta |
| 319 | 213629 | cttcgcttctctcttggaaagtccagtctctcctcggcttgcaatggaccccaactgctcctgcgccgctggtgtctcct |
| | _x_at | gcacctgcgctggttcctgcaagtgcaaagagtgcaaatgcacctcctgcaagaagagctgctgctcctngctnccccgt |
| | | gggctgtagcaagtgtgcccagggctgtgtttgcaaaggggcgtcagagaagt |
| | | gcagctgctgcgactgatgccaggacaacctttctcccagatgtaaacagagagacatgtacaaacctggattatttttt |
| | | ataccaccttgacccatttgctacattccttttcctgtgaaa |
| 320 | 213746 | cctggggctgagcaaggcctacgtaggccagaagagcagcttcacagtagactgcagcaaagcaggcaacaacatgctg |
| | _s_at | ctggtnggggttcatggcccnangaccccctgcgaggagnnnnngnngnagcacgtgggcagccggctntacagcgtg |
| | | tcctacctgctcaaggacaagggggagtacacactggtggtcaaatggggg |
| 321 | 213891 | gaaactgtatgggtagcttttttgtttgttttttgttttgtttttgtttttgtttttgtttttagttgtaggtcgcagcgg |
| | _s_at | ggaaattttttgcgactgtacacatagctgcagcattaaaaacttaaaaaaattgttaaaaaaanaaaaaaagggaaaacatt |
| | | tcaaaaaaaaaaaaanngataaacagttacaccttgttttcaatgtgtggctgagtgcctc |
| | | gattttttcatgtttttggtgtatttctgatttgtagaagtgtccaaacaggttgtgtgctggagttccttcaagacaaaaac |
| | | aaacccagcttggtcaaggccattacctgtttcccatctgtagttattcg |
| 322 | 213921 | gctgctgccgcggggaagcaggaactggccaagtacttcttggcagagctgctgtctgaacccaaccagacggagaatgatgccc |
| | _at | tggaacctgaagatctgtcccaggctgctgagcaggatgaaatgaggcttgagctgcagagatctgc |
| | | taactcaaacccggctatggcaccccgagaacgcaaagctggctgcaagaatt |
| | | tcttctggaagactttcacatcctgttagctttcttaactagtattgtccatatcagacctctgatccctcgcccccacacccc |
| | | atctctcttccctaatcctccaagtcttcagcgagacccttgcattagaaactga |
| 323 | 213953 | acaggaaattgctacttaccgccgccttctggaaggagaagacgtaaaaactacagaatatcagttaagcaccctggaagagag |
| | _at | agatataaagaaaaccagnaagattaagacagtcgtgcaagaagtagtggatggcaaggtcgtgtcatctgaagtcaa |
| | | agaggtggaagaaaatatctaaatagctaccagaaggagat |
| | | gctgctgaggttttgaaagaaatttggctataatcttatctttgctccctgcaagaaatcagccataagaaagcactattaat |
| | | actctgcagtgattagaaggggtggggtggcgggaatcctatttatcagactctgtaattgaatataaa |
| | | tgttttactcagaggagctgcaaattgcctgcaaaaatgaaatccagtgagcactagaatattt |
| | | aaaacatcattactgccatctttatcatgaagcacatcaattacaagctgtagaccacctaatatca |
| 324 | 214027 | gagaaagccaggcaggtagccagggggactagcccctgtggagactggggggcttgaaattgtccccgtggtctcttactttc |
| | _x_at | ctttccccagcccagggtggacttagaaagcaggggctacaagagggaatccccgaaggtgctggagg |
| | | tgggagcaggagattgagaaggagagaaagtgggtgagatgctggagaagagaggagaggagagaggcaga |
| | | gagcggtctcaggctggtgggaggggcgcccacctccccacgccctcccctcccctgctgcaggggctctggagaga |
| 325 | 214038 | atgagccttcatacatggactgagagtcagagcttgaagaaaagcttatttattttccccaacctcccccaggtgcagtgtg |
| | _at | acattattttattataacatccacaaagagattatttttaaataatttaaagcataatatttcttaaaaagtatttaatta |
| | | tatttaagttgttgatgttttaactctatctgtcatacatcctagtgaatgtaaaatgcaaa |
| | | atcctggtgatgtgttttttgtttttgttttcctgtgagctcaactaagttcacggcaaaatgtcattgttctccctcctac |
| | | ctgtctgtagtgttgtggggtcctcccatggatcatcaaggtgaaacactttggtattctttggcaatca |
| | | gtgctcctgtaagtcaaatgtgtgctttgtactgctgttgtt |
| 326 | 214091 | atgcctacaggtatgcgtgattgtgtgtgtgtgcatgggtgnacagccacntgtctacctatgtgtctttctgggaatgtgt |
| | _s_at | accatctgtgtgcctgcagctgtgtagtgctggacagtgacaaccctttctctccagttctccactccaatgataat |
| | | agttcacttacacctaaacccaaaggaaaaaccagctntaggtccaattgttntgctcta |
| | | nactgatacctcaaccttggggccagcatctcccactgcctccaaatattagtaantatgactgacgtncccanaagtttn |
| | | tgggtnnnaccacactccccaaccccccactcctanttcctgaagggccctcccaaggntacatccccaccc |
| | | cacagttctccctgagagagatcaacctccctgagatcaaccaaggcagatgtgac |
| | | agcaagggccacggacc |
| 327 | 214142 | gcctctggcaatgccattcaggccaggtcttcctcctatngtgnagannatnnnnntggnggtggaaagcgattctctcat |
| | _at | tctggcaaccagttggnacggccccatcaccgccctccgggtccgagtcaacacatactacatcgtaggtcttca |
| | | ggtgcgctatggcaaggtgtggagcgactatgtgggtggtcgcaacggagacc |
| | | tggaggagatcatctgcaccctggggaatcagtgatccaggtttctgggaagtacaagtggtacctgaagaagctggtatt |
| | | tgtgacagacaagggccgctatctgtcttttgggaaagacagtggcacaagtttcaatgccgtccccttgcaccccaac |
| | | accgtgctccgcttcatcagtggccggtctggttctctcatcgatgccattggcctgcactgggatgtttacccc |
| | | actagctgcagcagatgctgagcctcctctccttggcaggggcactgtgatgaggagtaagaactcccttatcactaac |
| 328 | 214164 | acaaggcccaggctggggccagggccagaggggaaggccctggattctcactcatgtgagatcttgaatctctttctttgt |
| | _x_at | tctgtttgtttagttagtatcatctggtaaaatagttaaaaaacaacaaaaaactctgtatctgtttctagcatgt |
| | | gctgcattgactctattaatcacatttcaaattcaccctacattcctacctcttcactagcctct |
| | | ctgaaggtgtcctggccagccctggagaagcactggtgtctgcagcacccctcagttcctgtgcctcagcccacaggccac |
| | | tgtgataatggtctgtttagcacttctgtat |
| 329 | 214414 | tcaacttcaagctcctaagccactgcctgctggtgaccctggccgcccacctccccgccgagttcaacccctgnggtgcacgc |
| | _x_at | ctccctgganaagttcctggcttctgtgagcaccgtgctgacctccaaataccgttaagct |
| | | ggagcctcggtagccgttcnnnnnncnngctnggcnntccaacgggccctcctcccctccttgcac |
| | | cggcccttcctggtctttgaataaagtctgagtg |
| 330 | 214433 | cttcctcggaggcagcattgttaagggaggccctgtgcaagtgctggaggacgaggaactaaagtcccagccagagcccctagt |
| | _s_at | ggtcaagggaaaacgggtggctggaggccctcagatgatccagctcagcctggatggg |
| | | aagcgcctctacatcaccacgtcgctgtacagtgcctgggacaagcagttttaccctgatctcat |
| | | cagggaaggctctgtgatgctgcaggttgatgtagacacagtaaaaggagggctgaagttgaaccccaacttcctggtggact |
| | | tcgggaaggagccccttggcccagcccttgcccnatgagctccgctaccctgggggcgattgtagctct |
| | | gacatctggatttgaaggctccaccctcatcacccacactccctattttgggccctcac |
| | | ttccttggggacctggcttcattctgctctctcttggcacccgacccttggcagcatgtaccacacagccaagctgagactgt |
| | | ggcaatgtgttgagtcatatacatttactgaccactgttgct |
| 331 | 214505 | ccatcggtgcggactccaaggaggtgcactataagaaccgcttctggcatgacacctgcttccgctgtgccaagtgccttcac |
| | _s_at | cccttggccaatgagacctttgtggccaaggacaacaagatcctgtgcaacaagtgcaccactcgggaggactc |
| | | ccccaagtgcaaggggtgcttcaaggccattgtggcaggagatcaaaac |
| | | gtggagtacaaggggaccgtctggcacaaagactgcttcacctgtagtaactgcaagcaagtcatcgggactggaagcttct |
| | | tccctaaaggggaggacttctactgcgtgacttgccatgagaccaagtttgccaagcattgcgtgaagtgc |
| | | aacaagggtttggtaaaggctccagtgtggtggcctatgaaggacaatcctggcacgactactgcttccac |
| | | tgcaaaaaatgctccgtgaatctggccaacaagcgctttgttttccaccaggagcaagtgtattgtcccgactgtgc |
| 332 | 214598 | ttgtcaaggggctttgcattcaaactgcttttccagggctatactcagaagaaagataaaagtgtgatctaagaaaaagtga |
| | _at | tggttttaggaaagtgaaaatatttttgtttttgtatttgaagaagaatgatgcattttgacaagaaatca |
| | | tatatgtatggatatattttaataagtatttgagtacagactttgaggtttcatcaatataaataaaag |
| | | agcagaaaaatatgtcttggttttcatttgcttaccaaaaaaacaacaacaaaaaaagttgtcctttgagaacttcacctgc |
| | | tcctatgtgggtacctgagtcaaaattgtcatttttgttctgtgaaaaataaatttccttcttgtaccatttct |
| | | gtttagttttactaaaatctgtaaatactgtatttttctgtttattccaaatttgatgaaactgacaatcca |
| | | atttgaaagtttgtgtcgacgtctgtctagcttaaatgaatg |
| 333 | 214677 | tcataagtgacttctacccgggagccgtgacagtggcctggaaggcagatagcagccccgtcaaggcgggagtggagacca |
| | _x_at | ccacaccctccaaacaaagcaacaacaagtacgcggccagcagctatctgagcctgacgcctgagcag |
| | | tggaagtcccacagaagctacagctgccaggtcacgcatgaagggagcacc |
| | | gtggagaagacagtggcccctacagaatgttcataggttctcaaccctcac |
| 334 | 214696 | gagtatacatcggtgcaggcttcctggatgacagttgggtgatatgtgtcatgtggcctaaaagcctccatgtcatttga |
| | _at | cctacgaattctatctttgggaatttatcctaagaaantacttanggatttanttngtgataagatgttcatcc |
| | | cagcattgcaatggagaaaaatgggaagcaatggtttggttgggaatttattccttttctgctgtaa |
| | | cgaaagtttgcaataggggattgcttaagtaaattattgtatctccatccagatggtggagtaccgcgcagacattaaaa |
| | | gtcatgtaaaagaacatctgactgaaagaaaaatgctccttgaatattaaaaggttgtaaaaatagtgcat |
| | | gttatgtgatttcaattttgttttttaaaatatgggtgtatgcttgtatacgtagagcagataaaaaagacgga |
| | | aggcatactaaaaaatgttgagtggttatctttgtatggtggaacaaagtcactgtaattttcatctttggtt |
| 335 | 214752 | tggggctgagcaaggcctacgtaggccagaagagcagcttcacagtagactgcagcaaagcaggcaacaacatgctgct |
| | _x_at | ggtgggggttcatggcccaaggaccccctgcgaggagatcctggtgaagcacgtgggcagccggctct |
| | | acagcgtgtcctacctgctcaaggacaagggggagtacacactggtggtcaaatg |
| | | gggggacgagcacatcccaggcagcccctaccgcgttgtggtgccctgagtntggggcccgtgccagccggcagcccc |
| | | caagcctgccccgctacccaagcagccccgccctnttcccctcaaccccggcccaggccgccctggccgccc |
| | | gcctgtcactgcagccgcccctgccctgtgccgtgntgcgctcacctgcctccccagccagccgctgacct |
| | | ctcggctttcacttgggcagagggagccatttggtggcgctgcttgtcttctttggttctgggaggggt |
| 336 | 214768 | aatgctctgggtctctggatccagtggggnatattgtgatgactcagtctccactctccctgcccgtcacccctggagagccg |
| | _x_at | gcctccatctcctgcaggtctagtcagagcctcntgcatnntaatggatacaactatttggattggtacctgcag |
| | | aagccagggcagtctccacagctcctgatctatttgggttctaatcgggcctccggggtcccngac |
| | | aggttcagtggcagtggatcaggcacagattttacactgaaaatcagcagagtggaggctgaggatgttggggtttattact |
| | | gcatgcaagctctacaaactcctcngacnttnggccangggaccaagntgganatcaaacgaactgtggctgcaccatct |
| 337 | 214777 | gcaagatggtgttgcagacccaggtcttcatttctctgttgctctggatctctggtgcctacggggacatcgtgatgacccag |
| | _at | tctccagactccctggctgtgtctctgggcgagagggccaccatcaactgcangtccagccagagtgttttatacagc |
| | | tccaacaataagaactacttagcttggtaccagcagaaaccaggacagcctcctaagctnctca |
| | | tttactgggcatctacccgggaatccggggtccctgaccgattcagtggcagcgggtctgggacagattt |
| 338 | 214916 | gaggtgcagctgttggagtctgggggaggcttggtacagcctggggggtccctgagactctcctgtgcagcctctggattc |
| | _x_at | acctttagcanctatgccatgagctgggtccgccaggctccaggnaaggggctggagtgggtctcanntattagtggt |
| | | agtggtnntancacatactacgnagactccgtgaagggccggttcaccatctccagagacaattccaagaacacgct |
| | | gtntctgcaaatgaacagcctgagagccgaggncacggccgtatattactgtncnnnagaaaatggtgtcagcagtt |
| | | gganntnccaaganannaccccaacctatgttgactactggggccaggaaccctggtcaccgtctcctcagggagtgcat |
| | | ccgccccaacccttttccccctcgtctcctgtgagaattccccgtcggatacgaagcagcgtggccgttggctgctc |
| 339 | 215049 | cgtcagtcatcctttattgcagtcgggatccttggggttgttctgttggccattttcgtcgcattattcttcttgactaa |
| | _x_at | aaagcgaagacagagacagcggcttgcagtttcctcaagaggagagaacttagtccaccaaattcaataccgggagatgaa |
| | | ttcttgcctgaatgcagatgatctggacctaatgaattcctcagaaaattcccatg |
| | | agtcagctgatttcagtgctgctgaactaatttctgtgtctaaatttcttcctatttctggaatggaaaaggaggccattct |
| | | gagccacactgaaaaggaaaatgggaatttataacccagtgagttcagcctttaagataccttgatgaagacctggac |
| | | tattgaatggagcagaaattcacctctctcactgactattacagttgcatttttatgga |
| | | gttcttcttctcctaggattcctaagactgctgctga |
| 340 | 215076 | ggggaatggagcaaaacagtctttgaatatcgaacacgcaaggctgtgagactacctattgtagatattgcaccctatgac |
| | _s_at | attggtggtcctgatcaagaatttggtgtggacgttggccct |
| 341 | 215118 | cttgctctgttgcagattggcagatgccgcctccctatgtggtgctggacttgccgcaggagaccctggaggaggagaccc |
| | _s_at | ccggcgccaacctgtggcccaccaccatcaccttcctcaccctcttcctgctgagcctgttctatagcacagc |
| | | actgaccgtgaccagcgtccggggcccatctggcaacagggagggcccccagtactgagcggga |
| | | gccggcaaggcacagggaggaagtgtggaggaacctcttggagaagccagctatgcttgccagaactcagcc |
| 342 | 215125 | gcataaattaatcagccccagagtgctttaannnnttctcttaaataccggtgtctttgatcaggatgacatgtgccattt |
| | _s_at | ttcagaggacgtgcagacaggctggcattctagattacttttcttactctgaaacatggcctgtttgggagtgcgg |
| | | gattcaaaggtggtcccaccgctgcccctactgcaaatggcagttntaatcttatcttttggct |
| | | tctgcagatggttgcaatngatccttaaccaataatggtcagtcctcatctctgt |
| 343 | 215176 | ctactctggctccgaggtgccagatgtgacatccagatgacccagtctccatcctccctgtctgcatctgtaggagacaga |
| | _x_at | gtcaccatcacttgccgggcaagtcagagcattagcagctatttaaattggtatcagcagaaaccaggnaaagcccctan |
| | | gctcctgatctatgctgcatccantttgcaaagtggggtcccatcaaggttcagtggcagtggatctgggacagatttca |
| | | ctctcaccatcagcagtctgcaacctgaagattttgcaacttactactgtcaacagagttacagtacccctca |
| 344 | 215193 | gccgagtactggaacagccagaaggacntcctggaagacnagcgggccnnggtggacacctactgcagacacaactacggg |
| | _x_at | gttgnngagagcttcacngtgcagcggcgagtccatcctnaggtgactgtgtatcctncaaagacccagcccctgca |
| | | gcaccacaanctcctggtctgntctgtgagtggtttctatccaggcagcattgnaagtcaggtggttncg |
| | | gaanggcnaggaagagaagnctggggntggtgtccacnggcctgatccagaatggagactggaccttccagaccctggt |
| | | gatgctngaaacagttcctcggagtggagaggtttacacctgccaagtggagcacccaagcntnacgagccctctcacagt |
| | | ggaatggagngcacggtctgaatctgcacagagcaagatgctgagtggagtcgggggctt |
| 345 | 215299 | aagatcctggagtttgtggggcgctccctnccagaggagacngtggacntcatggttnagcacacgtcgttcaaggagatg |
| | _x_at | aagaagaaccctatgaccaactacaccaccgtccnccnggagttcatggaccacagcatctcccccttcatgaggaa |
| | | aggcatggctggggacnngngnngnccacnttcaccgtggcgcaga |
| | | atgagcgcttcgatgcggacntatgcggagaagatggcaggncngcagcctcangcttccgctntgagcngtgagaggggn |
| | | nncntggagtcacngcagagggagtgtgcgaatcaaacctgaccaagcggntcaagaataaaatatgaattg |
| | | agggccngggacggtaggtcatgtctgtaatcccagcaatttggaggctgaggtgggag |
| | | gatcatttgagcccaggagttcgagaccaacctgggcaacatagtgagattctgttaaaaaaataaaataaaataaaacc |
| | | aatttttaaaaagagaataaaatatgattgtgggccaggcagagtggctcatgc |
| 346 | 215382 | ccggtcagcaggatcatcgtgcacccacagttctacatcatccagactggagcggatatcgccctgctggagctggagga |
| | _x_at | gcccgtgaacatctccagccgcgtccacacggtcatgctgccccctgcctcggagaccttccccccgggn |
| | | ntgccgtgctgggtcactggctggggcgatgtggacaatgatgagcccctcccaccg |
| | | ccatttcccctgaagcaggtgaaggtccccataatggaaaaccacatttgtgacgcaaaataccaccttggcgcctacac |
| | | gggagacgacgtccgcatcatccgtgacgacatgctgtgtgccgggaacacccggagngnntcatgcca |
| | | gggcgactcnggagggcccctggtgtgcaaggtgaatggcacctggctncaggcgggcgtggtcagctggg |
| | | ncgagggctgtgcccagcccaaccggcctggcatctacacccgtgtcacctactacttggactggatcc |
| 347 | 215388 | gaaccacctcaatgcaaagattctacgggaaaatgtgggccccctccacctattgacaatggggacattacttcattcccgttgt |
| | _s_at | cagtatatgctccagcttcatcagttgagtaccaatgccagaacttgtatcaacttgagggtaacaagcgaataacatgtag |
| | | aaatggacaatggtcagaaccaccaaaatgcttacatccgtgtgta |
| | | atatcccgagaaattatggaaaattataacatagcattaaggtggacagccaaacagaagctttatttgagaacaggtgaatc |
| | | agctgaatttgtgtgtaaacggggatatcngtctttcatcacgttctcacacattgcgaacaacatgttgggatgggnn |
| | | nctggagtatccaacttgtgcannnngatagaatcaatcataaaatgcacacctttattcagaactttagtattaaatcagt |
| | | tcttaatttcatttaagtattgttttactcctttttattcatacgtaaaattttggatta |
| 348 | 215657 | tcttccaatttattatctgcaccaattattttgggcacttctcctgtgtatgtaaactttggagtttaggtctacgtcattt |
| | _at | atataagagcctgtagacctgagtggatggacactgcctcttagaactagaacttagaactttatcttgaaaatgt |
| | | acnncnnnngcngaannnccncacagagtatgtgtcaggtaggaaccaaatttatgattgtt |
| | | atattcaaactggttttctgttccttgaaaatggtnttttttctgatcaaagcctatttaggagttattatgttcttgagt |
| | | ttattccatgtgtgtgaatattttgaatgaatccactaataagatatgcatgtatgtctggannnnnnncnnn |
| | | nnagaataaatatttaagtagactttaacaagtaaatggttgtaggcatcattaagtcttaaatagccttttc |
| | | aaagatattagtgttctngttttaattattcagataattcctttaattctccactcaaatctgacttta |
| 349 | 215867 | gattcctgtgggtccagctttggaactgggaaacctttcttcggatccgcactcattccactgatgccagctgcccctga |
| | _x_at | aggatgccagtactgtggtgtgtgagtctcagcagccgcccacacgctcctaactctgctgcatggcagatgcctaggtg |
| | | gaaatagcaaaaacaaggcccgggctggggccagggccagaggggaag |
| | | gccctggattctcactcatgtgagatcttgaatctctttctttgttctgtttgtttagttagtatcatctggtaaaata |
| | | gttaaaaaacaacaaaaaactctgtatctgtttctagcatgtgctgcattgactctattaatcacatttcaaattcaccc |
| | | tacattcctctcctcttcactagcctctctgaaggtgtcctggccagccctggagaagcactggtgtct |
| | | gcagcacccctcagttcctgtgcctcagcccacaggccactgtgataatggtctgtttagcacttctgtat |
| 350 | 216207 | ctctggctcccaggtgccagatgtgccatccagttgacccagtctccatcctccctgtctgcatctgtaggagacaga |
| | _x_at | gtcaccatcacttgccgggcaagtcagggcattagcagtgctttagcctgntatcagcagaaaccagggaa |
| | | agctcctaagctcctgatctatgatgcctccagtttgnaaantggggtcccatcaaggttcag |
| | | cggcagtggatctgggacagatttcactctcaccatcagcagcctgcagcctgaagattttgc |
| 351 | 216336 | ccaactgcctgactgcttgaggcctcaccggtgggagctccagcatctcctttgctcgaaatggaccccaactgctcc |
| | _x_at | tgtgccactggtggctcctgcatgtgcgccggctcctgcaagtgcaaagagtgcaaatgcacctcct |
| | | gcaagaagagctgctgctcctgctgccccgtgggctgtgccaagtgtgcccagggctgcatctgca |
| | | aaggggcgttggagaagtgcaactgctatgcctgacgtggggacagctctgctcccagatgtaaatagagtaacct |
| | | gcacaaacctggattttttaaaaaatacaacactgagccatttgcttcatt |
| 352 | 216401 | tcctccctgtctgcatctgtaggagacagagtcaccatcacttgccgggtgagtcagggcattagcagttatttaaattgg |
| | _x_at | tatcggcagaaaccagggaaagttcctaagctcctgatctatagtgcatccaatttgcaatctggagtccca |
| | | tctcggttcagtggcagtggatctgggacagatttcactctcactatcagcagcctgcagcct |
| | | gaagatgttgcaacttattacggtcaacggacttacaatgccc |
| 353 | 216442 | tggaggaagttctccagcttcagctcaactcacagcttctccaagcatcaccctgggagtttcctgagggttttctcataa |
| | _x_at | atgagggctgcacattgcctgttctgcttcgaagtattcaataccgctcagtattttaaatgaagtgattctaagattt |
| | | ggtttgggatcaataggaaagcatatgcagccaaccaagatgcaaatgttttgaaatgatatgaccaaaattt |
| | | taagtaggaaagtcacccaaacacttctgctttcacttaagtgtctggcccgcaatactgtaggaacaagcatg |
| | | atcttgttactgtgatattttaaatatccacagtactcactttttccaaatgatcctagtaattgcctagaaata |
| | | tctttctcttacctgttatttatcaatttttcccagtatttttatacggaaaaaattgtattgaaaacacttagtatgca |
| | | gttgataagaggaatttggtataattatggtgggtgattattttttatactgtatgtgccaaagctttactactgtg |
| 354 | 216474 | ccgccatttcctctgaagcaggtgaaggtccccataatggaaaaccacatttgtgacgcaaaataccaccttggcgcctacacg |
| | _x_at | ggagacgacgtccgcatcgtccgtgacgacatgctgtgtgccgggaacacccggagggactca |
| | | tgccagggcgactccggagggcccctggtgtgcaaggtgaatggcacctggctgcagg |
| | | cgggcgtggtcagctggggcgagggctgtgcccagcccaaccggcctggcatctacacccgtgtcacctactacttggactg |
| | | agatccaccactatgtccccaaaagccgtgagtcaggcctgggttggccacctgggtcactggaggaccaa |
| 355 | 216491 | agtcgggcccaggactggtgaagccttcggagaccctgtccctcacctgcactgtctctggtggctccatcagtagttacta |
| | _x_at | ctggagctggatccggcagcccgccgggaagggactggagtggattgggcgtatctataccagtgggagcacc |
| | | aactacaacccctccctcaagagtcgagtcaccatgtcagtagacacgtccaagaacca |
| | | gttctccctgaagctgagctctgtgaccgccgcggacacggccgtgtattactgtgcga |
| 356 | 216510 | agtgggtctcagctattagtgggagtgcaggtaccacatactacgcagactccgtgaagggccggttcaccacctccagaga |
| | _x_at | caattccaagaacacgctgtatctgcaaatgnacagnctgagagccgaggacacggccgtatattactgtgcgaaatccc |
| 357 | 216576 | tcctccctgtctgcatctgtaggagacagagtcaccatcacttgccaggcgagtcaggacattagtaattttttaacttgg |
| | _x_at | tatcagaagaagccagggaaagcccctaaagtcctgatctacgatgcatccaatttggaaacaggggtccca |
| | | tcaaggttcagtggaagtggttctgggacagattttactttcaccatcgccagcctgcagcctgacgattt |
| | | tgcaacatattactgtcaacaatataatgatcaccccctcactttcggccctgggaccaaagtg |
| 358 | 216834 | atgaaactgattacaacaggctgtaagaatcaaagtcaactgacatctatgctacatattattatatagtttgtactgagct |
| | _at | attgaagtcccattaacttaaagtatatgttttcaaattgccattgctactattgcttgtcggtgtatttta |
| | | ttttattgtttttgactttggaagagatgaactgtgtatttaacttaagctattgctcttaaaaccagggatca |
| | | gaatatatttgtaagttaaatcattggtgctaataataaatgtggattttgtattaaaatatatagaagcaatttctgttt |
| | | acatgtccttgctacttttaaaaacttgcatttattcctcagatttt |
| 359 | 216984 | catctcctgcactggaaccagcagtgacgttggtgcttataactatgtctcctggtaccaacagcacccaggcaaagcccc |
| | x_at | caaactcatgatttatgaggtcagtaatcggccctcaggggtactaatcgcttctctggctccaagtctggca |
| | | acacggcctccctgaccatctctgggctccaggctgacgacgagggtgattactactg |
| | | catctcatatacaagtagcaaccctctcgtggtt |
| 360 | 217022 | tcaagtgggaagagcgctgttcaaggaccacctgagcgtgacctctgtggctgctacagcgtgttccagtgtcctgccgg |
| | _s_at | gctgtgccgagccatggaaccatggggagaccttcacttgcactgctgcccaccccgagttgaagacc |
| | | ccactaaccgccaacatcacaaaatccggaaacacattccggcccgaggtccacctgct |
| | | gccgccgccgtcggaggagctggccctgaacgagctggtgacgctgacgtgcctggcacgtggcttcagcccaaggatgt |
| | | gctggttcgctggctgcaggggtcacaggagctgccccgcgagaagtacctgacttgggcatcccggcag |
| | | gagcccagccagggcaccaccaccttcgctgtgaccagcatactgcgcgtggca |
| | | gccgaggactggaagaagggggacaccttctcctgcatggtgggccacgaggccctgccgctggccttcacacagaag |
| | | accatcgaccgcttggcgggtaaacccacccatgtcaatgtgtctgttgtcatggcgga |
| 361 | 217109 | gctgtcaggattcttcaaggagaatgaatactgggaatcaagacaggactataccttatccataggcgcaggtgcaca |
| | _at | gggggaggccataaagatcaaacatgcatggatgggtcctcacgcagacacacccacagaaggacactagc |
| | | ctggcgcgcgtgcacacacacacacacacacacgagttcataatgtggtgatggc |
| | | cctaagttaagcaaaatgcttctgcacacaaaactctctggtttacttcaaat |
| 362 | 217110 | gcacctgagcatgaaactcgacgcgttcttcggcatcttctttggggccctgggcggcctcttgctgctgggggtcgggacgtt |
| | _s_at | cgtggtcctgcgcttctggggttgctccggggccaggttctcctatttcctgaactcagctgaggccttgcctt |
| | | gaaggggcagctgtggcctaggctacctcaagactcacctcatccttaccgcacattta |
| | | aggcgccattgcttttgggagactggaaaagggaaggtgactgaaggctgtcaggattct |
| 363 | 217148 | catctcctgcactggaaccagcagtgacgttgntggttataacnatgtctcctggtaccaacannacccaggcaaagccccc |
| | _x_at | aaactcatgatttatgnngtcagtaatcggccctcaggggtttctaatcgcttctctg |
| 364 | 217165 | gcgctggttcctgcaagtgcaaagagtgcaaatgcacctcctgcaagaagagctgctgctcctgctgccccgtgggctgtagc |
| | _x_at | aagtgtgcccagggctgtgtttgcaaaggg |
| 365 | 217179 | aggacagagggtcaccatctcttgctctggaagcagctccaacattgggaagaattatgtatcctggtaccagcaactcccag |
| | _x_at | gaacagcccccaaactcctcatctataaaaataataagcgaccctcagggattcctgaccgattctct |
| | | ggctccaagtctggcacgtcagccaccctgggcatcaccagactccagactggggac |
| | | gaggccgattattactgcggaacatgggatagcagcctgagtggtggccgggggatat |
| 366 | 217232 | tgaggagaagtctgccgttactgccctgtggggcaaggtgaacgtggatgaagttggtggtgaggccctgggcaggctgctg |
| | _x_at | gtggtctacccttggacccagaggttctttgagtcctttggggatctgtccactcctgatgctgttat |
| | | gggcaaccctaaggtgaaggctcatggcaagaaagtgctcggttctttgagtcctttggggatct |
| | | gtccactcctgatgctgttatgggcaaccctaaggtgaaggctcatggcaagaaagtgctcggtgcctttagtgatggcctg |
| | | gctcacctggacaacctcaagggcacctttgccacactgagtgagctgcactgtgacaagctgcacgtggat |
| | | cctgagaacttcaggctcctgggcaacgtgctggtctgtgtgctggcccatcactttg |
| | | gcaaaga |
| 367 | 217235 | cagtgtccggatctcctggacagtcagtcaccatctcctgcaccggaaccagcagtgatgttggtggttataactatgtctc |
| | _at_x | ctggtaccaacagcacccaggcaaagcccccacactcatgatttatgatgtcattaagcggccctcaggggtccctg |
| | | atcgcttctctggctccaaatctggcaacacggcctccctgaccatctctgggct |
| | | ccaggctgaggatgaggctgattattattgcagctcaaatgcaggcagtttttatgtct |
| 368 | 217258 | tcctgcactgggagcagctcctacatcggggcaggttatgatgtacactggtaccggcaacttcccggcacagcccccaaa |
| | _x_at | ctcctcatctatggtaacaccgatcggccctcaggggtccctgaccgattctctggctccaagtctggcatctca |
| | | gcctccctggccatcactgggctccaggctgaggatgaggctgattattactgcaa |
| | | gtcctatgacagcagcctgag |
| 369 | 217378 | gatgtgacatccaggtgacccagtctccatcttccctgtctgcgtctgtaggagacagagtcaccatcacctgccgggcaagtca |
| | _x_at | gggcattagcaatgggttatcctggtatcagcagaaaccagggcaagcccctacgctcctgatctatgctg |
| | | catccagtttgcagtcgggggtcccatctcggttcagtggcagtggatctgggaca |
| | | gatttcactctcaccatcagcagcctgcagcctgaagatgttgc |
| 370 | 217414 | ggtccccacagactcagagagaacccaccatggtgctgtctcctgccgacaagaccaacgtcaaggccgcctggggtaaggtcg |
| | _x_at | gcgcgcacgctggcgagtatggtgcggaggccctggagaggcacttcgacctgagccacggctctgc |
| | | ccaagttaagggccacggcaagaaggtggccgacgcgctgaccaacgcc |
| | | gtggcgcacgtggacgacatgcccaacgcgctgtccgccctgagcgacctgcacgcgcacaagcttcgggtggacccggtcaa |
| | | cttcaagctaagccactgcctgctggtgaccctggccgcccacctccccgccgagttcacccctgcggtgc |
| | | acgcttccctggacaagttcctggcttctgtgagcaccgtgctgacctccaaat |
| | | accgttaagctggagcctcggtagcc |
| 371 | 217480 | gctgccaggtgccagatgtgacatccagatgacccagtctccatcctccctgtctgcatctgtaggagacagagtcaccatca |
| | _x_at | cttgccgggcgagtcagggcattagcaataatttaaattggtatcagcagaaaccagggaaaactcctaagctcct |
| | | gatctatgctgcacccagtctgcaaagtgggattccctctcggttcagtgaca |
| | | gtggatctggggcagattacactctcaccatccgcagcctgcagcctgaagattttgc |
| 372 | 217546 | agtcgctccatttatcgcttgagatctccagccttaccgcggctcgaaatggaccccaactgctcctgcaccactggtgtct |
| | _at | cctgcgcctgcaccggctcctgcaagtgcaaagagtgcaaatgcacctcctgcaagaagagctgctgctcctgctgcc |
| | | ccgtgggctgtgccaagtgtgcccacggctgtgtctgcaaagggacgttggagaactgcagctgctgtgcctgatgtggga |
| | | acagctcttctcccagatgttaatagaacaagctgcacaacctggattttttttcaatacgatactgagccatttgct |
| 373 | 217757 | cctccagacctccttgaaatacaatattctcccagaaaaggaagagttcccctttgctttaggagtgcagactctgcctcaa |
| | _at | acttgtgatgaacccaaagcccacaccagcttccaaatctccctaagtgtcagttacacagggagccgctctgc |
| | | ctccaacatggcgatcgttgatgtgaagatggtctctggcttcattcccctgaagcc |
| | | aacagtgaaaatgcttgaaagatctaaccatgtgagccggacagaagtcagcagcaaccatgtcttgatttaccttgataa |
| | | ggtgtcaaatcagacactgagcttgttcttcacggttctgcaagatgtcccagtaagagatctcaaaccagc |
| | | catagtgaaagtctatgattactacgagacggatgagtttgcaatcgctgagtacaatgctccttgca |
| 374 | 217762 | ctattaccattctaactttcataaaaagttgggatcaagaagcagctgatttcctgccagggcttatattagggggntgatt |
| | _s_at | cttaaaggacattaggattggtgctcagaaatggttaatcatgctgtgtgctagccagggccagctggtaccttctttgcca |
| | | tgagcattcaagggacngctaacctttattgacaatctatatngcaaaagtca |
| | | ggaaagaggttgtgagctgattggattaaagacctggcacttcagtaactcagcacgcttccacttcactcaacttaagaga |
| | | gttcattgacagtgttaggatgtgaaggctgggaaacacttattttgcttcaagagttccacttggctctccc |
| | | aaataggtacctcaaaaactgttagcaagcggcatttggatgtcttgacaggggctttgcagggat |
| | | ttttagggttttttccacattgtccacattaatggttggcatgattgtgcttgcaggccaag |
| 375 | 217764 | gaatatgacgttaccttgcagactaaaaggttgaaggcccgaaactaacttttagctaacaataagggctgtgccccaatgga |
| | _s_at | aactgagttcattttctgagaaaggtttggatgactgaaatatttcctctacagtcaaggactttggcatgcg |
| | | gtggctgaaactgagcttttttgtgtgggctccagttctcactgttctgcaatgctcatggc |
| | | aagttgaatggtgagctagcttataaattaaagagctctgaactgtattcagaccgactgggtatctagcttactgttttaa |
| | | catcattgttgaaaccagaccctgtagtccagtggtgctgccctgttgtgcaaactgctcctttttctcgtgtttttgtaa |
| | | agagcttccatctgggctggacccagttcttgcacatacaagacaccgctgcagtcag |
| | | ctaggacctttccgccatgtattctattctgtagtaaagcatttccatcaacaatgcctaattgtatctgttatttttggt |
| | | ttaacacacactgattcatact |
| 376 | 217767 | ggtctacgcctattacaacctggaggaaagctgtacccggttctaccatccggaaaaggaggatggaaagctgaacaagct |
| | _at | ctgccgtgatgaactgtgccgctgtgctgaggagaattgcttcatacaaaagtcggatgacaaggtcaccctg |
| | | gaagaacggctggacaaggcctgtgagccaggagtggactatgtgtacaaga |
| | | cccgactggtcaaggttcagctgtccaatgactttgacgagtacatcatggccattgagcagaccatcaagtcaggctcg |
| | | gatgaggtgcaggttggacagcagcgcacgttcatcagccccatcaagtgcagagaagccctgaagctggaggagaa |
| | | gaaacactacctcatgtggggtctctcctccgatttctggggagagaagcccaacctcagctacatcatcgggaaggaca |
| | | cttgggtggagcactggcctgaggaggacgaatgccaagacgaagagaaccagaaacaatgccaggacc |
| | | tcggcgccttcaccgagagcatggttgtc |
| 377 | 217897 | cccctgcatatcttctcagcaataactccatgggctctgggaccctaccccttccaaccttccctgcttctgagacttcaatct |
| | _at | acagcccagctcatccagatgcagactacagtccctgcaattgggtctctggcaggcaatagttgaaggactcctgttcc |
| | | gttggggccagcacaccgggatggatggagggagagcagaggcctttgcttctctgcctacgtccccttagatgggcagcagag |
| | | gcaactcccgcatcctttgctctgcctgtcggtggtcagagcggtgagcgaggtgggttggagactcagc |
| | | aggctccgtgcagcccttgggaacagtgagaggttgaaggtcataacgagagtgggaactcaacccagatcccgcccctcctgt |
| | | cctctgtgttcccgcggaaaccaaccaaaccgtgcgctgtgacccattgctgttctctgtatcgtgatctatcctcaacaaca |
| 378 | 217967 | ctcttttactactgacctttcacagagaaaaaatatttcccttgaaaaaaactgggcttgtcattttttcccttgtagctttaa |
| | _s_at | gcagagacataagtgccttgcattacacatagtaaactttctttaaaaaaaaaaaaaaaagattttggagactaccagggtaa |
| | | gattccaacttgtccaaaagctttctggccttacatattttattataaaaattctcaagtctggtaatcttctatgtcagagct |
| | | agtgatttcaaaaggtttcacaattccccaagacaaaagtgattttcgttcattataataaggttaagtgatatgtgattcat |
| | | aacaattttgatgtgaagaagggaaggacatcattgacttaataatagtatcagtcggtgcaacagttggcaacatgtgcctt |
| | | cacactttaccataaagagacgggtttgagggtttgccttctaaagtctgcaacttcaagaaaaaaaatcgacactgtggatt |
| | | gactttcccggt |
| 379 | 218087 | aactttgtatagcccatgtacctaccttgtatagaaaaataattttaaaaatttgaatggaagggggtaaaggaagtcatgaag |
| | _s_at | tttttttgcatttttatttaaatgaaggaattccaaataactcacctacagatttttagcacaaaaatagccattgt |
| | | aaagtgttaaaatttacgataagtattctattggggaggaaaggtaactctgatctcagt |
| | | tacagtttttttttcctttttaatttcattattttgggtttttggtttttgcagtcctatttatctgcagtcgtattaagtcc |
| | | tattgctagaataggttactacaaaaaaggttatattctgaaagaaaaataactgacattatatataaccaattaatttaaag |
| | | tattgccatttaaattacacactgagagcatgtcctatgcagacatagatttttctgttcatttatttt |
| | | tcttcattgcagtggattgatttgataaatagatgtgttgaattactacatttgctgtacatat |
| 380 | 218162 | ggagctagccttgttEgcatctttctcactcccatacatttatattatatccccactaaatttcttgttcctcattcttcaaat |
| | _at | gtgggccagttgtggctcaaatcctctatatttttagccaatggcaatcaaattctttcagctcctttgtttcatacggaac |
| | | tccagatcctgagtaatccttttagagcccgaagagtcaaaaccctcaatgttccctcct |
| | | gctctcctgccccatgtcaacaaatttcaggctaaggatgccccagacccagggctctaaccttgtatgcgggcaggcccagg |
| | | gagcaggcagcagtgttcttcccctcagagtgacttggggagggagaaataggaggagacgtccagc |
| | | tctgtcctctcttcctcactcctcccttcagtgtcctgaggaacaggactttctccacattgttttgta |
| 381 | 218224 | gcgggtcttgctggctaaaatgcccaggtaaagggttggttggacacagcgcttagtgcacgctgtcatcatggacatcataat |
| | _at | cagttgtgaaaaacacgcgaacctatgacacttcttattccacactgaatgtgaaattgcatgttcagatgtttn |
| | | actacgaggcctggctcacaggaagtgttcagtaaaagtatgcactgttagattac |
| | | tgataacgcggatagatttttgttnaccataaattgttccagatttatattaatggaaggaagtgtgcatttattagctatta |
| | | ntcaactttacaangcaaacatcttatttctcatctttaaacatgtcgaccagtttaattgaaaagtattctgagactg |
| | | caaaatggggtgttaaaaaatactgcagttacggagctgtgtaaaccagtttctcattgca |
| | | taagatacagatgtaaattgcatggagaggttgatatgcacctgtacagtaattcactcccccatttcacttctttgtcag |
| | | agaatagttcttgttcatactgagtgtt |
| 382 | 218312 | gcatccgaggggagccgccggatgtggaagaagactcggctttcctgcagccatttagtgccgccccatgctaggttattt |
| | _s_at | gacattgtgcagtgtagagttgccttaaagtgcgtgatctgccagtgctttcttcaagtcacccttgccccga |
| | | ttcctcatgtttgcgctccccagggttgctcaagtggaaattttgtcagctgtttagccttttcgt |
| | | acttggcgtgatgtcaacttcacttctaatctgcaaaagcagaagctgtttcctagtttacctcgcgtgtgtttacctata |
| | | tggagtagctcgcagagatcacagaaatgcttgcagcctaaggcagggttttcagaccgtgggtcccagccca |
| | | tttagtaaaatgggaaatcaattagcaagtggtcaccagcattacacagca |
| 383 | 218353 | tatacagtttaataagcctcttgcaagttacttgttctctcacctgaggtatttttttcctccccaccttgcccctgttcct |
| | _at | cccttcctcttctccctttgcaagaggaaatatttaacatatttgggtccaacttcaataatgtaataattaatacattaaa |
| | | agcatttaacttcctttctagaaaaatgcacaggctaaggcatagacaaaacaaagaga |
| | | aatgctgagaaatttgccactggagacaagcaatctgaataaatatttgccaaaagttctttttatgtcatatagtgtcagg |
| | | atttgaaggagctattttttttaatgttgcaactagcaactcatcttcggaagacacagccaggagaatgaagtagaagtga |
| | | aaggtttataaatccatttgtaagcatttatcccatatattttaaattcaagaaaaattgtgtttatctttagaangtat |
| | | tcaatactttatgtactatgtgactcatgcttctggataaataaagcaccaaatatgtatctgtaaccacaatcacacata |
| 384 | 218418 | ggcttgatgtagactggcttgctttgatttttagtgaagggaatgtacgtaaaacaaaatagggcttggctggtcaaaggag |
| | _s_at | acaagcaggatggatggatggatggatgaatagatagatggtgtttgcatgtaaattgcagagaaaacaaaaccaaag |
| | | ctgattggaaacaattaattgtgggtgtctgagggggaaggtcgcagcttt |
| | | gggcagctttgagaagcggtacaagagctctgtgcctgtgtgtccagccctggagccagccagtgcatttattttaagctc |
| | | ttagaagcaactccttggcccaggaatgcgtgacccctgagatgggtccacgcatctctctacacgtccttctctcc |
| | | gtgggatactggactcgtgcctctgcgcccattctcttctcacgcatatccatgagct |
| | | ttaatttcactttctgatcacggtacgtccataaagccagt |
| 385 | 218468 | ggagatgacttaagttggcagcagtaatcttcttttaggagcttgtaccacagtcttgcacataagtgcagatttgccccaag |
| | _s_at | taaagagaatttcctcaacactaacttcacggggataatcaccacgtaactacccttaaagcatatcactagccaaag |
| | | aggggaatatctgttcttcttactgtgcctatattaagactagtacaaatgtggtgtgtcttccaactttc |
| | | attgaaaatgccatatctataccatattttattcgagtcactgatgatgtaatgatatattttttcattattatagtag |
| | | gaatatttttatggcaagagattttggtcttgatcatacctatt |
| 386 | 218469 | gaagctgacacaccgtatgttgttagagtcttttatctggtcaggggaaacaaaatcttgacccagctgaacatgtcttcctga |
| | _at | gtcagtgcctgaatctttattttttaaattgaatgttccttaaaggttaacatttctaaagcaatattaagaaagac |
| | | tttaaatgttattttggaagacttacgatgcatgtatacaaacgaatagcagataatgat |
| | | gactagttcacacataaagtccttttaaggagaaaatctaaaatgaaaagtggataaacagaacatttataagtgatcagtta |
| | | atgcctaagagtgaaagtagttctattgacattcctcaagatatttaatatcaactgcattatgtattatgtct |
| | | gcttaaatcatttaaaaacggcaaagaattatatagactatgaggtaccttgctgtgtagg |
| | | aggatgaaaggggagttgatagtctcataaaactaatttggcttcaagtttcatgaatctgtaactagaatttaattttcacc |
| | | ccaataatgttctatatagcctttgctaa |
| 387 | 218541 | gttcaaaatatttccaagcctgagtattgtctattggtatagatnagaaatcaataattgattatttatttgcacttattac |
| | _s_at | aatgcctgaaaaagtgcaccacatggatgttaagtagaaattcaagaaagtaagatgtcttcagcaactcagtaaaac |
| | | cttacgccaccttttggtttgtaagaggttttttatacatttcaaacaggttgcacaaa |
| | | agttaaaataatggggtcttttataaatccaaagtactgtgaaaacattttacatattnaaatcttctgactaatgctaaaa |
| | | cgtaatctaattaaatttcatacagttactgcagtaagcattaggaagtgaatatgatatacaaaatagtttata |
| | | aagactctatagtttctataatttattttactggcaaatgtcatgcaacaa |
| 388 | 218546 | ggcagcccacaagtttctcgtggggagatggaggcagagcccagggtaggggacagagctgctggggcctttccttgcctgg |
| | _at | gaatctgtcccaggaagagcttccccactcccatcccccaaattggaaaaaccgtacattcaagcctgtttggcc |
| | | ctgaaattcttaagaatctggttaagaattaactcactaatgtcaaaagtcaa |
| | | aacctcctaggggttgtcctgggagtcaggttcacgggtacagaagatgaatctcagatgtcactcaacctgagccgtcat |
| | | tctctgtggcagggctgccctgggtttctcttactcaatccctggagtgtaagcatttggattgtgtcacagattacctt |
| | | tttaccttttctttctttttttttctttttttcaatatcagtgcccacaccttactgagtattgagtt |
| | | ttagagctttcgcttgatgtgcttgaccaagagacttcttttgtatccttttct |
| 389 | 218559 | tcaagtgcgttctttagaccaatgcattgcgtttctttccctgcttttgagatagtaggaagagttcttggtggtgtcc |
| | _s_at | ccccccttcaattcttcagttgtatagtagttatagggaagatatgggtgatttctttattattacttttttttttct |
| | | gcaggtcagtaaaaggatttaagttgcactgacaaaaataccaaaataaaagtgtatttttaagttcccattt |
| | | gaaattgctggcgctgctggccggatgcatttttgagtttgtattagttgataaattaacagtaataacaagattgta |
| | | tgaaccgcatggtgcttgcagttttaaatattgtggatatttgtcctgcatcagaaacgagctttggttttta |
| | | cagattcaactgtgttgaaatcaaacctgccgcaacagaaattgtttttatttcatgtaaaataagggatca |
| | | atttcaaaccctgcttatg |
| 390 | 218756 | cctcctgcctctggattttaggtgttgatttctggatcacgggataccacttcctgtccacaccccgaccaggggctagaaaa |
| | _s_a | tttgtttgagatttttatatcatcttgtcaaattgcttcagttgtaaatgtgaaaaatgggctggggaaaggaggtggt |
| | | gtccctaattgttttacttgttaacttgttcttgtgcccctgggcacttggcctttgtctgctct |
| | | cagtgtcttccctttgacatgggaaaggagttgtggccaaaatccccatcttcttgcacctcaacgtctgtggctcagggct |
| | | ggggtggcagagggaggccttcaccttatatctgtgttgttatccagggctccagacttcctcctctgcctgc |
| | | cccactgcaccctctcccccttatctatctccttctcggctccccagcccagtcttggcttcttgt |
| | | cccctcctggggtcatccctccactctgactctgactatggcagcag |
| 391 | 219014 | ggcatgtgtgactgtttcagcgactgcggagtctgtctctgtggcacattttgtttcccgtgccttgggtgtcaagttgcagc |
| | _at | tgatatgaatgaatgctgtctgtgtggaacaagcgtcgcaatgaggactctctacaggacccgatatggcatccctggatcta |
| | | tttgtgatgactatatggcaactctttgctgtcctcattgtactctttgccaaatc |
| | | aagagagatatcaacagaaggagagccatgcgtactttctaaaaactgatggtgaaaagctcttaccgaagcaacaaaattca |
| | | gcagacacctcttcagcttgagttcttcaccatcttttgcaactgaaatatgatggatatgcttaagtacaactgatgg |
| | | catgaaaaaaatcaaatttttgatttattataaatgaatgttgtccctgaacttagctaaatggtgcaacttagt |
| | | ttctccttgctttcatattatcg |
| 392 | 219059 | agagaatgcccttctccttattgtaaccctgtctggatcctatcctcctacctccaaagcttcccacggcctttctagcctggct |
| | _s_at | atgtcctaataatatcccactgggagaaaggagttttgncaaagtgcaaggacctaaaacatctcatcagtatccagtgg |
| | | taaaaaggcctcctggctgtctgaggctaggtgggttgaaagccaagga |
| | | gtcactgagaccaaggctttctctactgattccgcagctcagaccctttcttcagctctgaaagagaaacacgtatcccacctg |
| | | acatgtccttctgagcccggtaagagcaaaagaatggcagaaaagtttagcccctgaaagccatggagattctcat |
| | | aacttgagacctaatctctgtaaagctaaaataaagaaatagaacaaggctgaggatacgacagtacact |
| | | gtcagcagggactgtaaacacagacagggtcaaagtgttttctctgaacacattgagttggaatcactgtt |
| | | tagaacacacacacttactttttctggtctctaccactgctgatattttct |
| 393 | 219087 | attaccttatcatgtcttagagcccgtctttatgtttaaaactaatttcttaaaataaagccttcagtaaatgttcattacca |
| | _at | acttgataaatgctactcataagagctggtttggggctatagcatatgcttattttttttaattattacctgatttaaaaat |
| | | ctctgtaaaaacgtgtagtgtttcataaaatctgtaactcgcattttaatgatccgctattataa |
| | | gcttttaatagcatgaaaattgttaggctatataacattgccacttcaactctaaggaatatttttgagatatccctttgg |
| | | aagaccttgcttggaagagcctggacactaacaattctacaccaaattgtctcttcaaatacgtatggactggataactctg |
| | | agagacacatctagtataactgaataagcagagcatcaaattaaacagacagaaaccgaaagctctatataaatgctcagag |
| | | ttctttatgtatt |
| 394 | 219508 | atggggctggggacttgaattggatgcttcaaaaccatcacctgttggccaacaagtttgacccaaaggtagatgataatgctct |
| | _at | tcagtgcttagaagaatacctacgttataaggccatctatgggactgaactttgagacacactatgagagcgttgctacctgtgg |
| | | ggcaagagcatgtacaaacatgctcagaacttgctgggacagtgtgggtgggagaccagggctttgcaattcgtggcatccttt |
| | | aggataagagggctgctattagattgtgggtaagtagatcttttgccttgcaaattgctgcctgggtgaatgctgcttgttctct |
| | | cacccctaaccctagtagttcctccactaactttctcactaagtgagaatgagaactgctgtgatagggagagtgaaggaggga |
| | | tatgtggtagagcacttgatttcagttgaatgcctgctggtagcttttccattctgtggagctgccgttcctaataattccag |
| 395 | 219543 | ggaattcttctcttacttcaataaaatgggttttaacataactttaaattcagttaaatatacaatattgaatacctatagttg |
| | _at | actttgggatggggactttttcaagtcattaagagtgtttgtttaaggtgatctcattgatggtagttctcagccgtctca |
| | | aaaactgcaagctaatcagtcagacattctttaatgaccccaattttttcactttaattgttaccatgttttctattttt |
| | | actgatttttgctaaagcatgtaagagtgaatttattatagcagtaatcttgtgtttctcctgatgtgca |
| 396 | 219607 | atcaacacatttagcttggcgttttattcattccatcacccttactgtaactactatggcaactcaaataattgtcatgg |
| | _s_at | gactatgtccatcttaatgggtctggatggcatggtgctcctcttaagtgtgctggaattctgcattgctgtgtccctct |
| | | ctgcctttggatgtaaagtgctctgttgtacccctggtggggttgtgttaattctgccatcacattctcacatggcag |
| | | aaacagcatctcccacaccacttaatgaggtttgaggccacccaaagatcaacagacaaatgctccagaaatctatgctg |
| | | actgtgacacaag |
| 397 | 219669 | catctctcaggaggtgggctgtccaccaaaatgagcattcagggctgcgtggcccaaccttccagcttcttgttgaaccacacca |
| | _at | gacaaatcgggatcttctctgcgcgtgagaagcgtgatgtgcagcctcctgcctctcagcatgagggaggtggggctgagg |
| | | gcctggagtctctcacttggggggtggggctggcactggccccagcgctgtggtggggagtggtttgcccttcctgctaactct |
| | | attacccccacgattcttcaccgctgctgaccacccacactcaacctccctctgacctcataacctaatggccttggacacca |
| | | gattctttcccattctgtccatgaatcatcttccccacacacaatcattcatatctactcacctaacagcaacactggggaga |
| | | gcctggagcatccggacttgccctatgggagag |
| 398 | 219796 | tccttgtccacaagcactatggcccccggctcaagtgctgctctggcaaagctccggagccccagccccaaggctttgacaac |
| | _s_at | caggcgttcctccctgaccacaaggccaactgggcgcccgtccccagccccacgcacgaccccaagcccgcggaggcaccgat |
| | | gcccgcagagcccgcaccccccggccctgcctccccaggcggtgcccctgagccccccgcagcggcccgagctggcggaag |
| | | ccccacggcggtgaggtccatcctgaccaaggagcggcggccagagggcgggtacaaggctgtctggtttggcgagg |
| | | acatcgggacggaggcagacgtggtcgttctcaacgcgcccaccctggacgtggatggcgccagtgactccggcagcg |
| | | gcgatgagggcgagggcgcggggaggggtgggggtccctacgatgcgcccggtggtgatgactcctacatctaagtggccc |
| 399 | 219799 | accatcgctggtggtatcccagggtccctgctcaagttttctttgaaaaggagggctggaatggtacatcacataggcaagt |
| | _s_at | cctgccctgtatttaggctttgcctgcttggtgtgatgtaagggaaattgaaagacttgcccattcaaaatgatctttac |
| | | cgtggcctgccccatgcttatggtccccagcatttacagtaacttgtgaatgttaagtatcatctcttatctaaatatt |
| 400 | 219948 | gaatactcataattcttatctctataatcaaaagtataatttactgtagaaaaataaagagatgcttgttctgaaagtaag |
| | _x_at | atcagtgaactgcttttcagtctcaatctttgagaattgtaaattcatcaaataattgcttacatagtaaaaatttaaggt |
| | | attagaaaacctgcataacaaatagtattatatattaaatattttgatatgtaaagctcta |
| | | cacaaagctaaatatagtgtaataatgtttacactaataagcaaatatgttaatcttctcatttttttactgtcatataat |
| | | cttagtgatatgcctattaatagttttaaataaataaattggctcatctggctttttgaaaattttgaaattcttacagat |
| | | gttgattaggtatatctacaaattaatttcaattttaaaatgatgatataaaaataaatataagtatt |
| | | tttcttgtgtatgtata |
| 401 | 220026 | agtaactttgtttatccctcaagcaaatcctgatgacattgatcctactcctactcctactcctactcctgataaaagtcataa |
| | _at | ttctggagttaatatttctacgctggtattgtctgtgattgggtctgttgtaattgttaactttattttaagtacca |
| | | ccatttgaaccttaacgaagaaaaaaatcttcaagtagacctagaagagagttttaaaaaaca |
| | | aaacaatgtaagtaaaggatatttctgaatcttaaaattcatcccatgtgtgatcataaactcataaaaataattttaagatg |
| | | tcggaaaaggatactttgattaaataaaaacactcatggatatgtaaaaactgtcaagattaaaatttaatagtt |
| | | tcatttatttgttattttatttgtaagaaatagtgatgaacaaagatcctttttcatactgatac |
| | | ctggttgtatattatttgatgcaacagttttctgaaatgat |
| 402 | 220037 | ttccacttctattccacggagaaaaaaattgatttgtgtcacagaagatttatggaaactagcaccatgtctacagaaactgaa |
| | _s_at | ccatttgttgaaaataaagcagcattcaagaatgaagctgctgggtttggaggtgtccccacggctctgctagtgctt |
| | | gctctcctcttctttggtgctgcagctggtcttggattttgctatgtcaaaaggtat |
| | | gtgaaggccttcccttttacaaacaagaatcagcagaaggaaatgatcgaaaccaaagtagtaaaggaggagaaggccaatga |
| | | tagcaaccctaatgaggaatcaaagaaaactgataaaaacccagaagagtccaagag |
| | | tccaagcaaaactaccgtgcgatgcctgaagctgaagtttagatga |
| 403 | 220075 | ctcaactcggccatcacatatcgaattaccaaccactcacacttccggatggagggagaggttgtgctgaccaccaccacac |
| | _s_at | tggcacaggcgggagccttctacgcagaggttgaggcc |
| 404 | 220266 | tccattaccaagagctcatgccacccggttcctgcatgccagaggagcccaagccaaagaggggaagacgatcgtggccccgg |
| | _s_at | aaaaggaccgccacccacacttgtgattacgcgggctgcggcaaaacctacacaaagagttcccatctcaaggcacacc |
| | | tgcgaacccacacaggtgagaaaccttaccactgtgactgggacggctgtggatggaaattcgcccgctcagatgaactg |
| | | accaggcactaccgtaaacacacggggcaccgcccgttccagtgccaaaaatgcgaccgagcattttccaggtcggacc |
| 405 | 220376 | aaaacccattctttcagttacatctactaagagctcgtagttcaataacattccaaagaacgtagtttggaaaacactggcctat |
| | _at | cggctgtaataggaagggtctaaagaaaattatttgctgagtcctcataatcaattggctataatcacaatttaa |
| | | taatttatcaatgcaaacagtacattaaaaactgtagcacaatctataatttttacctt |
| | | catatgcagagaaattaagaatttacatcttttacaaatatgtgtatctctgaaataatgaaatatattgatgggtgaaataat |
| | | atttctggtattttcttcattataacacgtggaatgggacagtggctagaggtatggatggagcaaggtcaaatatgag |
| | | ctgatggagctgggcgctaaataagggtataatatgctacttctgtgtatgttgtgtatggtatagtatgctatt |
| | | tctgtgtatgttcaaattgtttatgtatataataaagttttaaaaatttgtatgtacttgacaattccttagaa |
| 406 | 220468 | agaggagacactttggcgttcttcaagcagaactgaggctgcgaaaaatccaagtctctacagagacactgatgaagttgaaagg |
| | _at | gtaattgtttttccatgccaaatgaggaaatcaaattaatgagttgacaaacttttcctgagatgttatttcatctac |
| | | atttagttaaacaacttagaatgatatctagaaaatatttattttcagccagga |
| | | actttagcaaactgtggcaatgatcactgggaatgaaaacgtataatgttctacaatttttgttatcacattggatgactttga |
| | | atatagtagtgacacttgggaccaaataaattattttatatgactactagaacaaagttttagtaagctgtctgctaata |
| | | gagtcagaattcttccagattgtttccagctgtaacaggtattgaatttccaccgtgctcctatgtaacagctgggtg |
| | | gtgtaaaaaatgaacttcaaattatggagtggaagaagcgtaatgttaatatcttgtaaattcgtattccctat |
| 407 | 220645 | cattttccaggatctcagtgtgagtatcattgatgcctgggatataacaattgcatatggcacaaataatgtacacccacctca |
| | _at | acatgtagtcggaaatcagattaatatattattaaactatatttgttaaataacacaaaagtctgaaattcattcact |
| | | taagtaaaaaaatttattgactgtctactagcaggccagatgctgtgtttggctctgaattcccaactagcaagagcag |
| | | agaatctacattatggctgatccataagccaccaattcagctaaatgagatgtttctaatctgggcttccacttaaaaa |
| | | taaaactaaaaatactcatgaaaagagcctacccttcttgatcagagccatacccctcttcaccaatgggaagt |
| 408 | 220812 | ggtattttagtctagttttatatgaacggttgtatcagggtaaccaactcgatttgggatgaatcttagggcaccaaagact |
| | _s_at | aagacagtatctttaagattgctagggaaaagggccctatgtgtcaggcctctgagcccaagccaagcatcgcatccc |
| | | ctgtgatttgcacgtatacatccagatggcctaaagtaactgaagatccacaaa |
| | | agaagtaaaaatagccttaactgatgacattccaccattgtgatttgttcctgccccaccctaactgatcaatgtacttt |
| | | gtaatctcccccacccttaagaaggtactttgtaatcttccccacccttaagaaggttctttgtaattctccccaccct |
| | | tgagaatgtactttgtgagatccaccctgcccacaaaacattgctcttaacttcaccgcctaa |
| | | cccaaaacctataagaactaatgataatccatcacccttcgc |
| 409 | 220834 | gctggccaagactactgggccgtgctttctggaaaaggcatttcagccacgctgatgatcttctccctcttggagttcttcg |
| | _at | tagcttgtgccacagcccattttgccaaccaagcaaacaccacaaccaatatgtctgtcctggttattccaaatatgtatg |
| | | aaagcaaccctgtgacaccagcgtcttcttcagctcctcccagatgcaacaac |
| | | tactcagctaatgcccctaaatagtaaaagaaaaaggggtatcagtctaatctcatggagaaaaactacttgcaaaaactt |
| | | cttaagaagatgtcttttattgtctacaatgatttctagtctttaaaaactgtgtttgagatttgtttttagg |
| | | ttggtcgctaatgatggctgtatctcccttcactgtctcttcctacattaccactactacatgctggcaa |
| | | aggtgaaggatcagaggactgaaaaatgattctgcaactctcttaaa |
| 410 | 221004 | agcccaccagcaggagcttggagtttggggagtggggatgagtccgtcaagcacaactgttctctgagtggaaccaaag |
| | _s_at | aagcaaggagctaggacccccagtcctgccccccaggagcacaagcagggtcccctcagtcaaggcagtggga |
| | | tgggcggctgaggaacggggcaggcaaggtcactgctcagtcacgtccacgggggacgagccgtgggttctgctgag |
| | | taggtggagctcattgctttctccaagcttggaactgttttgaaagataacacagagggaaagggagagccacctg |
| | | gtacttgt |
| 411 | 221305 | gccactatcttgaagaaggtgcacagtgccctgctcctctttcctatgtccccagaattctcttagggttctcagatgccat |
| | _s_at | gactttcaaggagagagtacggaaccacatcatgcacttggaggaacatttattttgccagtatttttccaaaaatgcccta |
| | | gaaatagcctctgaaattctccaaacacctgtcacagcatatgatctctacagccacacatcaatttggttgttgcgaac |
| | | agactttgttttggactatcccaaacccgtgatgcccaatatgatcttcattggtggtatcaactgccatcagggaaagcc |
| 412 | 221541 | ctttatcatccccacaaacattttgaaactggaatatttgtcttcagaaaatggaaacaagactataaatgataagccctg |
| | _at | tccctagcaccacctctcctgtgtgtggaatagaggcccctcgtgctaccaacacttaccctgtgtttaaaaagat |
| | | cttgtaccaagccaacggcgttcctggctctcctgcccacaggatgaacattttcggcttccttaggagttttgc |
| | | cctaccgtattccaaagcgtgtgctggtttctcatattgtctgtaggctcac |
| 413 | 221584 | gtgaggctcagtcagaacctccaccctcccccacaccaaagacaggggcagcgtagtattcaaaccagtattgtggtggggaat |
| | _s_at | aattgtatacatgtaaattatcaagccctatgagtggaagaattttttcaaattatttttgtctctctatatattgattt |
| | | atattatgtataactatctctttatataaactatatataattatatatatataactatat |
| | | aattatatatatataactatatatataactatatatatgtatcccctagtattggatcatgaagagctcttcatgcattcttt |
| | | gcaaaggaggttataaagttacgccctcagaacatttataactataagaatgtgccagttaaagtgctcaacaggaaata |
| | | tgacagtttaaaagcattgtaaaactcacatagcttacttctctctctaaagtgcaacaaggatgaatagaatgggcca |
| | | aggtatgacaattaatggttctgcatgacctagccactgctgggggttttcttctataacgttgtccttgtga |
| 414 | 221667 | gggacttaacatttcacgttgtatcttacttgcagtgaatgcaagggttacttttctctggggacctcccccatcacccagg |
| | _s_at | ttcctactctgggctcccgattcccatggctcccaaaccatgccgcatggtttggttaatgaaacccagtagctaaccccac |
| | | tgtgcttccacatgcctggcctaaaatgggtgatatacaggtcttatatccccat |
| | | atggaatttatccatcaaccacataaaaacaaacagtgccttctgccctctgcccagatgtgtccagcacgttctcaaagt |
| | | ttccacattagcactccctaaggacgctgggagcctgtcagtttatgatctgacctaggtcccccctttcttc |
| | | tgtcccctgtttttaagtccggatttttacagaaggaactgtctccagacagctcatcaaggaaccaagcaa |
| | | aggccagatagcctgacagataggctagtggtaattgtgtatatgggcgggacgtgtgtgtcatta |
| 415 | 221747 | cctctgtcctcaaatgtccaaaatgttggaggacctctgttcatatcccacgcctgggctcttgccagcagtggagttactgtaga |
| | _at | gggatgtcccaagcttgttttccaatcagtgttaagctgtttgaaactctcctgtgtctgtgttttgtttgtgcgtgtgtgtgag |
| | | agcacatcagtgtgtgcaggctgtgtttccccatttctctcctcccttcagacccatcattgagaacaaatgtaagaaatcc |
| | | cttcccaccaccctccctgcctcccaggccctctgcgggggaaacaagatcacccagcatcct |
| 416 | 221748 | atattttgtatcatcgtgcctatagccgctgccaccgtgtataaatcctggtgtntgctccttatcctggacatgaatgtattgta |
| | _s_at | cactgacgcgtccccactcctgtacagctgctttgtttctttgcaatgcattgtatggcttta |
| 417 | 221841 | atccgacttgaatattcctggacttacanaatgccaagggggtgactggaagttgtggatatcagggtataaattatatccgtgag |
| | _s_at | ttgggggagggaagaccagaattcccttgaattgtgtattgatgcaatataagcataaaagatcaccttgtattctctttaccttc |
| | | taaaagccattattatgatgttagaagaagaggaagaaattcag |
| | | gtacagaaaacatgtttaaatagcctaaatgatggtgcttggtgagtcttggttctaaaggtaccaaacaaggaagccaaagttt |
| | | tcaaactgctgcatactttgacaaggaaaatctatatttgtcttccgatcaacatttatgacctaagtcaggtaatatac |
| | | ctggtttacttctttagcatttttatgcagacagtctgttatgcactgtggtttcagatgtgcaataatttgtacaatg |
| | | gtttattcccaagtatgccttaagcagaacaaatgtgtttttctatatagttccttgccttaa |
| 418 | 221896 | ccaatcctgaaggtactccctgtttgctgcagaatgtcagatattttggatgttgcataagagtcctatttgccccagttaattc |
| | _s_at | aacttttgtctgcctgttttgtggactggctggctctgttagaactctgtccaaaaagtgcatggaatataacttgtaaag |
| | | cttcccacaattgacaatatatatgcattgttttaaaccaaatccagaaagcttaa |
| | | acaatagagctgcataatagtatttattaaagaatcacaactgtaaacatgagaataacttaaggattctagtttagtttttgt |
| | | aattgcaaattatatttttgctgctgatatattagaataatttttaaatgtcatcttgaaatagaaatatgtattttaa |
| | | gcactcacgcaaaggtaaatgaacacgttttaaatgtgtgtgttgctaattttttccataagaattgtaaacattgaac |
| | | tgaacaaattacctataatggatttggttaatgacttatgagcaagctggttggccagacagtatacccaaacttttat |
| | | ataatatacagaaggctatcacacttgtgaa |
| 419 | 222043 | tcgactctgctgctcatgggaagaacagaattgctcctgcatgcaactaattcaataaaactgtcttgtgagctgatcgcttg |
| | _at | gagggtcctctttttatgttgagttgctgcttcccggcatgccttcattttgctatggggggcaggcaggggggatggaaa |
| | | ataagtagaaacaaaaaagcagtggctaagatggtatagggactgtcataccagtgaagaataaaagggtgaagaataaa |
| | | agggatatgatgacaaggttgatccacttcaagaattgcttgctttcaggaagagagatgtgtttcaacaagccaacta |
| 420 | 222162 | aataacgcaaatggcttcctctttcctttttttggaccatctcagtctttatttgtgtaattcattttgaggaaaaaacaact |
| | _s_at | ccatgtatttattcaagtgcattaaagtctacaatggaaaaaaagcagtgaagcattagatgctggtaaaagctagaggag |
| | | acacaatgagcttagtacctccaatttcctttctttcctaccatgtaaccctgctttgggaatatggatgtaaagaagt |
| | | aacttgtgtctcatgaaaatcagtacaatcacacaaggaggatgaaacgccggaacaaaaatgaggtgtgtagaacagg |
| | | gtcccacaggtttggggacattgagatcacttgtcttgtggtggggaggctgctgaggggtagcaggtccatctcc |
| | | agcagctggtccaacagtcgtatcctggtgaatgtctgttcagctcttct |
| 421 | 222453 | tcaaatggaacctgccctctaaagcactttctttcctttacttgcgtggtttcatgtaagctgtgctgtttagaaacaacatctc |
| | _at | agactttacaaagaaatgacaaagaaggcaattgcactttttaagggatatcgacaagcagtttctgttttctaaaggacaaaa |
| | | tacagagtgtgtgtcatttttaattagattctttcccctgctgagttggaaattccagtgcagcactgattgaccacagttgc |
| | | caatctaaaagcacaaagacagaagtaaagctttatgctaattttatttcaatatgatagaaaatttatcttggtatgtc |
| | | cttttttagataactccagcaggaaactgtaactgctatgtctttaggaaaacgtagaagaaagaacattattattcttta |
| | | attcctacaaggtacttgaaaaccttaagtgaaaaagatttctatctttttatcttggcgcatt |
| 422 | 222513 | tgccactaattcattcacactaaggtgtaaatgattgataataggaatgagttacctcttcccacagacatttgtattaagtat |
| | _s_at | gacagagcagggccttaatcccaagggaaaaggttatggaactggagggggtgagctttctgggtagaaggagacttcctga |
| | | atttccttaaaacccagtaagagtaagacctgttgttttggaaggtctgctccaccatctaagagcactgattttttttttt |
| | | gttgttgttgttgttttacggtctctgagggaatatagtaaaaatgcatatgcacgtgcaatttgcacggcagcatttca |
| | | ccgattgtggactgtattggctaatgtgtttcctggtctttagatgcaaaccattaataacactatcttatctcatagttt |
| | | tttcaggggtgcttcttgattagtagggaattttgaacacctctttaaatacagctagaaaataaaaccaatttgtaaagcc |
| | | acatttgcatatgatgccagcctcacgcatttgtatatctccagaaattcaggtatgcctcaccaatttgcccgtctttaataa |
| 423 | 222717 | taaactaacccttacattccatgtgatgtgatgtaggcttataagntgctaaaatctataggtntnggaagtgaaagattctatt |
| | _at | ttttcttttttngtacataatgggaatttcattccagattatattttatttacatattaatttcacagaatattaatatttctta |
| | | acttcttaaagcatgctagcgttttatgtatatgtacacatatattcagacagggtaatttt |
| | | atctgctgcctaacattgtactaaaatattgctttatctgtttttaattacaaaatgctaatgatttcttaaattatagtttaaa |
| | | gacaattggcccaggaagcaaatcccctgcctttagtatgaaccactataagtaaccttacaaatagagttaatccaagacaat |
| | | attaacaaactgtgctttgtctttaataaaagggataggattaacaaacatattgatggcataacctattcagctatgtcctt |
| | | atttttgcaataatgtaacctcaaatatggattgttgaaccaacaatggctgtgttaaa |
| 424 | 222722 | tttatgatgactcagtggtgccagagtaaagtttctaaaataacattcctctcacttgtaccccactaaaagtattagnctaca |
| | _at | cattacattgaagttaaacacaaaattatcagtgattagaaacatgagtccggactgtgtaagtaaaagtacaaacatta |
| | | tttccaccataaagtatgtattgaaatcaagttgtctctgtgtacagaatacatacttattcccatttttaagcatttgc |
| | | ttctgttttccctacctagaatgtcagatgtttttcagttatctccccatttgtcaaagttgacctcaagataacatttt |
| | | tcattaaagcatctgagatctaagaacacaattattattctaacaatgattattagctcattcacttattttg |
| | | ataactaatgatcacagctattatactactttctcgttattttgtgtgcatgcctcatttc |
| | | cctgacttaaacctcactgagagcgcaaaatgcagctttatactttttacttt |
| 425 | 223121 | gataacctacatcaaccgagataccaaaatcatcctggagaccaagagcaagaccatttacaagctgaacggtgtgtccgaaa |
| | _s_at | gggacctgaagaaatcggtgctgtggctcaaagacagcttgcagtgcacctgtgaggagatgaacgacatcaacgcgccctat |
| | | ctggtcatgggacagaaacagggtggggagctggtgat |
| | | cacctcggtgaagcggtggcagaaggggcagagagagttcaagcgcatctcccgcagcatccgcaagctgcagtgctagtccc |
| | | ggcatcctgatggctccgacaggcctgctccagagcacggctgaccatttctgctccgggatctcagctcccgttcccca |
| | | agcacactcctagctgctccagtctcagcctgggcagcttccccctgccttttgcacgtttgcatccccagcatttcct |
| | | gagttataaggccacaggagtggatagctgttttcacctaaaggaaaagcccacccgaatcttgtagaaat |
| 426 | 223122 | ggtgcaactgtgacttgggtctggttggttgttgtttgttgttttgagtcagctgattttcacttcccactgaggttgtcata |
| | _s_at | acatgcaaattgcttcaattttctctgtggcccaaacttgtgggtcacaaaccctgttgagataaagctggctgttatctcaa |
| | | catcttcatcagctccagactgagactcagtgtctaagtcttacaacaattcatcattttataccttcaatgggaacttaaac |
| | | tgttacatgtatcacattccagctacaatacttccatttattagaagcacattaaccatttctatagcatgatttcttcaag |
| | | taaaaggcaaaagatataaattttataattgacttgagtactttaagccttgtttaaaacatttcttactta |
| | | acttttgcaaattaaacccattgtagcttacctgtaat |
| 427 | 223235 | agggtacgagaacttgccaatgggaaattcatccgagtggcactggcagagaaggataggagtggaatgcccacacagtgacc |
| | _s_at | aacagaactggtctgcgtgcataaccagctgccaccctcaggcctgggccccagagctcagggcacccagtgtcttaagg |
| | | aaccatttggaggacagtctgagagcaggaacttcaagctgtgattctatctcggctcagacttttggttggaaaaa |
| | | gatcttcatggccccaaatcccctgagacatgccttgtagaatgattagtgatgttgtgatgcttgtggagcat |
| | | cgcgtaaggcttcttgcttatttaaactgtgcaaggtaaaaatcaagcctttggagccacag |
| 428 | 223343 | gagtccaaatgtcatcagtgctcattttgagataccctgctatcgatggtcgctacaaaccaggaaatactcaagttatta |
| | _at | tgtgtatacattggntttagntttatgaaacaatttaccttcatgatctcatagttaaaattgtaataaatttaggaat |
| | | ataaaggatcaatatgggaagcaaaatttctaaaggcagtttctgttgttttaattagtatttgtgtagttcaaacc |
| | | aggaaggatttgactatcattagattttgcttaactttatgaaagctaaaatattctctgttataaaggggcaactccat |
| | | ctggtcctatagcatctttactactgatttttnngtttaatttgaaaatgcaaagaattgttaaatgttcttaaa |
| | | tgttctcactacaaaaaaagaaaaaagataactacgtgaggtgatggatatgttaattagctggat |
| | | tgtggtaatcattttggaatgtatatgtatatcaaaacatgtagtacaccctaaatatat |
| 429 | 223395 | agatacatttttacccaccataaatgttacaatatctgaatatgctttgtcaaactatccctttatgcaatcgtcttcata |
| | _at | ttgtttttatgattctaatcaagctgtatgtagagactgaatgtgaagtcaagtctgagcacaaaaagataatgcacg |
| | | atgagattgcctaccattttataggatatttactatgtatttatacgttaagacctctatgaatga |
| | | atgtatcagagaatgtctttgtaactgtttaattcaatctgtaataaaaatctaactaactaactcatttatttctattaaa |
| | | aaggtattgtcctttaggcggggaatgggaatccttgctgcactgttgcagtcattctgaaaggacctttccct |
| | | gtacttacctttcaacatgcttcaatcttatcaacgctacat |
| 430 | 223484 | gaaaaccgatgtgatccttgatcgaaaaaaaaatccagaaccttgggaaactgtggaccctactgtacctcaaaagctta |
| | _at | taacaatcaaccaacaatggaaacccattgaagagttgcaaaatgtccaaagggtgaccaaatgacgagccctcgcctc |
| | | tttcttctgaagagtactctataaatctagtggaaacatttctgcacaaactagattctggacaccagtgtgcggaaa |
| | | tgcttctgctacatttttagggtttgtctacattttttgggctctggataaggaattaaaggagtgcagcaataactg |
| | | ccactgttaaaagtttgtgcttattttcttgtaaatttgaatattgcatattgaaatttttgtttatgatctat |
| | | gaatgtttttcttaaaatttacaaagctttgtaaattagattttctttaataaaatgccatttgtgcaagatttct |
| 431 | 223551 | aattgtgttgtgatgctactcactttgattgcaatgatgatgtccaaggtaagctattaaaaggcaggttacttccaaatcgcactg |
| | _at | aaggaaaaggttaagaataatacatgatcacagaaatgcataccactgtctgtaaacccaacaaaattcactgttctcttttggatt |
| | | tatttagcctgatgtatttttaattcaatttttatggtgatgggcaaatcattcttggtaaatgtaaatcaaacatgattgatttaa |
| | | aacttcatggaatttgtagaaaattatggacatttttggtgagaaagaacaatagtcaaaactcacatggatagagtgtgtttgt |
| | | tttttgccaaaaatgccccagaccttttcccaaacctcaaaaacgtcttggaaaaattgtaaaagtttgataacagaaacat |
| | | ctttaggatatttttgtctgacgtattttgcttctagtatgtgcctactgtgatttttttcatgtggaaaatgcaaaa |
| | | tttgtaacaaaatggttatatggaacatgcctattaaacgaa |
| 432 | 223597 | ggcatctggcacgtgcccaataagtcccccatgcagcactggagaaacagctccctgctgaggtaccgcacggacactggcttcc |
| | _at | tccagacactgggacataatctgtttggcatctaccagaaatatccagtgaaatatggagaaggaaagtgttggactgacaac |
| | | ggcccggtgatccctgtggtctatgattttggcgacgccca |
| | | gaaaacagcatcttattactcaccctatggccagcgggaattcactgcgggatttgttcagttcagggtatttaataacgagag |
| | | agcagccaacgccttgtgtgctggaatgagggtcaccggatgtaacactgagcaccactgcattggtggaggaggatactt |
| | | tccagaggccagtccccagcagtgtggagatttttctggttttgattggagtggatatggaactcatgttggtt |
| | | acagcagcagccgtgagataactgaggcagctgtgcttctattcta |
| 433 | 223623 | gagaagcacctgttccaactaagactaaagtggccgttgatgagaataaagccaaagaattccttggcagcctgaagcgccaga |
| | _at | agcggcagctgtgggaccggactcggcccgaggtgcagcagtggtaccagcagtttctctacatgggctttgacgaagcga |
| | | aatttgaagatgacatcacctattggcttaacagagatcgaaatggacatgaatactatggcgattactaccaacgtcact |
| | | atgatgaagactctgcaattggtccccggagcccctacggctttaggcatggagccagcgtcaactacgatgactacta |
| | | accatgacttgccacacgctgtacaagaagcaaatagcgattctcttcatgtatctcctaatgccttacactacttggttt |
| | | ctgatttgctctatttcagcagatctttctacctact |
| 434 | 223754 | agctgccgggcaacgtgttgtgtaagtgaacatctgggaggtaaacactacacgtgaagagtggtgaaagggaacattgatta |
| | _at | ctgaagtgccctggagagggaaagcactggtcaacatcacatggacaaatttcattgttttctaaagatggcctggaag |
| | | tagtctttgccactgcttcctccacaaacagctcttcataacatgggctgcatgaaatcaaagcaaactttcccatt |
| | | tcctaccatatatgaaggtgagaagcagcatgagagtgaagaaccctttatgccagaagagagatgtctacctaggat |
| | | ggcttctccagttaatgtcaaagaggaagtgaaggaacctccagggaccaatattgtgatcttggaatatgcacaccgcct |
| | | gtctcaggatatcttgtgtgatgccttgcagcaatgggcatgcaataacatcaagtaccatgacatt |
| | | ccatacattgagagtgaggggccttgaggctgtaggatgaca |
| 435 | 223952 | ggggctatactccatccaaatatgcagtggaaggtttcaatgacagcttaagacgggacatgaaagcttttggtgtgcacg |
| | _x_at | tctcatgcattgaacgtctagacaaactgaaaggcaataaatcctatgtgaacatggacctctctccggtggtag |
| | | agtgcatggaccacgctctaacaagtctcttccctaagactcattatgccgctgga |
| | | aaagatgccaaaattttctggatacctctgtctcacatgccagcagctttgcaagactttttattgttgaaacagaaa |
| | | gcagagctggctaatcccaaggcagtgtgactcagctaaccacaaatgtctcctccaggct |
| | | atgaaattggccgatttcaagaacacatctccttttcaacc |
| 436 | 224009 | ggggctatactccatccaaatatgcagtggaaggtttcaatgacagcttaagacgggacctgaaagcttttggtgtgcacgt |
| | _x_at | ctcatgcattgaaccaggattgttcaaaacaaacttggcagatccagtaaaggtaattgaaaaaaaactcgccatttgggag |
| | | cagctgtctccagacatcaaacaacaatatggagaaggttacattgaaaaaagtctagacaaactgaaaggcaat |
| | | aaatcctatgtgaacatggacctctctccggtggtagagtgcatggaccacgctctaacaagtctcttccctaaga |
| | | ctcattatgccgctggaaaagatgccaaaattttctggatacctctgtctcacatgccagcagctttgcaagacttttta |
| | | ttgttgaaacagaaagcagagctggctaatcccaaggcagtgtgactcagctaaccacaaatgtctcctccaggc |
| | | tatgaaattggccgatttcaagaacacatctccttttcaacc |
| 437 | 224342 | tatctgcggccccaggacagaaggtcgccatctcctgctctggaagcagctccaacattgggaataattatgtatcctggta |
| | _x_at | ccaacaactcccaggaacagcccccaaactcctcatttatgacaataataagcgaccctcagggattcctggccga |
| | | ttctctggctccaagtctggcacgtcagccaccctgggcatcaccggactccagactgaggacgaggccgat |
| | | tattactgcggaacatgggatagcagcctgag |
| 438 | 224352 | ttttatttcaggcttccagctgtccctgtgagttatcctggacatttcgatggtttttggtaaggccaaactctgataagcaaaa |
| | _s_at | cagagaatactgacggtatacttaaccatatgtgtaactgatacttggcaccatggaatttttcattgagttatttcctca |
| | | ttcnttaaaaaataagggactataaatcagttatttagtatcttttgtttttgtagctgattccttaactttcttgtatg |
| | | cctctagtaatttcagagattaaatattgctttaaactgtgatactttgatttgctagattgacaaaactgatactaatataat |
| | | taagttcatctttgaaatacatctttgtgcgtagagccaaaaaaagagataaaattaataatagttcacttgtt |
| | | atttgagattaatttggcatttgaaatgatcattttattttacaatcatttataatgaatcaatgttccagt |
| | | tagctttaaaaggtatacggtgctaattagtaaaatattgaaggcaatattttactgctagcttgca |
| 439 | 224412 | taatcctgacattagctgacttgctagtgagcttgctttaaaaatctacactcttgcattcttaggcatacaggggaaatgtt |
| | _s_at | gaaaaggaaggtggaaaaccaagaatttagtttgccaatgattgcctctgattcttgtaagtttgagttccacaagggc |
| | | taatttattccccttttacttgggttttggggtggtggaaagcgggaaatttgggtgatttgttgattggcaatg |
| | | aggataaaatgttaatacttttttggggacttaacaactttatcctattctacaagtcagtaaaggaacaattggt |
| | | actcacctcagtgctgcactcaactatggaaagaggcagagtttgcttgcccaattgccaaactaaa |
| 440 | 224480 | attagtgtcattagtgtgttggaagagaaatactattcagtaagcttcgccaaagaaaagtgagtcaaagttaatgtgtgtgt |
| | _s_a | gcatttatatgtaggcagctcgtagaccacattttagccagcaactggtaacaaagagcttagttttccttgtttgaatgctg |
| | | tagatctgtacctagtacccctcccatctactgatttgtttgtttttgtaaccaaac |
| | | acattttcagatagaaggagccttaaaaaaaaaaaaatcacattgagtaacttcagtatgaatgaatgagagtgtgtggagc |
| | | tacccctcaccctccacccctttgtgctttttattcccgaattttcccagtctcttaaacagaaaaatgactgatataa |
| | | ttatcttttggaaactgagccttaattttttttagagggggaaataagttttccccaactcacacagcataagcaatg |
| | | tttgacagcaatataatgccgttgtaaactactgagagtattgtatctgttctggtaaccatgtaca |
| 441 | 224560 | ttgtttttgacatcagctgtaatcattcctgtgctgtgttttttattacccttggtaggtattagacttgcacnttttttaaa |
| | _at | aaaaggtttctgcatcgtggaagcatttgacccagagtggaacgcgtggcctatgcaggtggattccttcaggtctttcc |
| | | tttggttctttgagcatnctttgctttcattcgtctcccgtctttggttctccagttcaaattattgcaaagtaaagg |
| | | atctttgagtaggttcggtctgaaaggtgtggcctttatatttgatccacacacgttggtcttttaaccgtgctgagcag |
| | | aaaacaaaacaggttaagaagagccgggtggcagctgacagaggaagccgctcaaataccttcacaat |
| 442 | 224663 | ggtcagatggtcaacttttttcagtattatttatagttggcacttgattgcagttctgtgaggcttgagcattcatacacctcac |
| | _s_at | ctgccttggcaagcctattttagtgatatggcagcacggatataacactatgcattaaaagcactttttgtaataagtttaata |
| | | tcctaaaaggaatgccaattaagttttgttaactgtgtcatcaacttatcctagta |
| | | cctcagtgttcattcctgttacctgcatatcttcttaaaagaaatagctgttattaatgcctttttgttttccattgagtgt |
| | | acactactgaataagtgtaggagttttatgtttaccatgtgagtcctgcaacactaaagatattttgaatatcagtcatgat |
| | | ggcaatttctgtataaaagagccttaaatggaacattgttttgagatcaaactccccaccc |
| 443 | 224694 | gatatctggccatgggtaacctcattgtaactatcatcagaatgggcagagatgatcttgaagtgtcacatacactaaagtccaa |
| | _at | acactatgtcagatgggggtaaaatccattaaagaacaggaaaaaataattataagatgataagcaaatgtttcagcccaatgtc |
| | | aacccagttaaaaaaaaaattaatgctgtgtaaaatggttg |
| | | aattagtttgcaaactatataaagacatatgcagtaaaaagtctgttaatgcacatcctgtgggaatggagtgttctaaccaatt |
| | | gccttttcttgttatctgagctctcctatattatcatactcagataaccaaattaaaagaattagaatatgatttttaatacact |
| | | taacattaaactcttctaactttcttctttctgtgataattcagaagatagttatggatcttcaatgcctc |
| | | tgagtcattgttataaaaaatcagttatcactataccatgctat |
| 444 | 224823 | gacctaatacatttcctctgtgtgtgtgtgtaacattccaaatactttttttttcttttccactgtttgtaaggtgcaacaattt |
| | _at | aatatttttaagggactttttaagagttccttaagaaccaatttaaaattacttcagtgcaatcctacacagtatcaacatta |
| | | gaattttgatattagtcttatgttatcttccattctatttttatctgctttttgctgctagtttcaaac |
| | | tgccagtatttttccttttgcttttaaaatagttacaatatttttcatgatagccacagtattgccacagtttattataataa |
| | | agggtttttatttgatttagcgcattcaaagcttttttctatcacttttgtgttcagaatataacctttgtgtgcgtgtatgt |
| | | tgtgtgtgtgcatgtgtggcgtatatgtgtgttacaggttaatgccttcttggaattgtgttaatgttctcttggttta |
| | | ttatgccatcagaatggtaaatgagaacactacaactgtagtcagctcacaatttt |
| 445 | 224836 | gagtgagtcccagttacatcaaacagtgacttccagttattccccagtaagtctgagtggttccttcaagctgggtgtctttcc |
| | _at | agcctttgccagtctagccccagcagggcaccgtgtatgaatgcagtttggtgctgttttagagtatgcctgctccccagcccc |
| | | ctgcctggaaccctctgagcaacttgctctgacctataatgtcttaggtgcaacacggaccccaccagagctcttggatacc |
| | | cccctagatccatgtggctttatgtgaggggactgaatgcagacacaccatagcccccttctactactttccctctcgccct |
| | | gccacctagttccacatggaaccaacaagttgagtgcatccctgttgggtgttttgtgttgagactggctgaaatgaggagac |
| | | tttgaccatgtgacgtgtcaacagactcaaggagacaaccacctcaactgggtcatgtg |
| 446 | 224840 | ccccagtctcacctacgaaatatgaagagcaaaagctgattttgcttacttgctaaactgttgggaaagctctgtagagcatg |
| | _at | gttccagtgaggccaagattgaaatttgatactaaaaaggccacctagctttttgcagataacaaacaagaaagctattcc |
| | | aagactcagatgatgccagctgtctcccacgtgtgtattatggttcaccagggggaactggcaaaagtgtgtgtggg |
| | | gaggggaagggtgtgtgagtggttctgagcaaataactacagggtgcccattaccactcaagaagacacttcacgt |
| | | attcttgtatcaaattcaataatcttaaacaatttgtgtagaagtccacagacatctttcaaccaccttttaggctgcata |
| | | tggattgccaagtcag |
| 447 | 224959 | tcatgcgtcatccctaaaataataagatacatgggatcaaatagcccttgccttttcaacacaaatcagttggaaaattatggtt |
| | _at | tgagtcctgttgctgccatggcttctgtttctcagaaatgagtgtgtatgaacataccaatctatgtaataggctacctttttt |
| | | tgtcttctttggaactttgtacacaaaccaagacaatatcagggtgacaggtgaatgaacttaaattctcagtcttgtctattc |
| | | accaaaaaagtatactgcctgttttttctttaattattcaaggttgatgacttttaggaacatgttttatactgtattttttaa |
| | | ttaaagcaagtgccttgatgtaattccatgtaaatcattgcttaaccctcttatgggatgaggatgagttattaatgtattgca |
| | | gcctactggaaaggagggggagttggttaatagcagatacttttcttctagaagcttatgttttatgctgtttattatgtaaga |
| | | tcctgtatgtgtgttgaga |
| 448 | 224963 | ataatgatccattcgagttctgtgatccttattgttcttaattgtgtttctctacgtattgttacagatgagccatacgtttc |
| | _at | tttgtatcaatgtagacatgacttcagatacctctgaggacctacccagcagtctaggaccctgggccaagtgctggga |
| | | ctatggtactaaatccagtagatgggctgtgtagcaactctcccagggaacacactag |
| | | ggtacttagggaggtgctttgtggagcatgttgaagctttgagatctgagcaggaggcagtgatgtccctggtctattca |
| | | gggaaagatttcagtgtgaaatggtaaacatccaattgacaggatttagattttgcttagtttttctgctt |
| | | tttaatgtttctatcccccatctcagtgttttctttatccatcccagtgatgcct |
| 449 | 224964 | ataattttacatgatcctcaatatcaactccagtttaaaaagtgttatttttaaaacatttgaaaccaagtactgtttaatt |
| | _s_a | tcaatcagaagatgcaaatacatactttgatctatgtttgattttgctaataatatttgaaggagattgcctaccaagg |
| | | acaaaacaataaatttaaaaatcaaacgatttctccatacgctcatagtcacatatggaatt |
| | | ttgagaaaataaagcatgctgtctttaggaatttttatacttctttgtctttcttccttaatatttgcttctagctgctc |
| | | ttggcaatgatgaattgttatgtatgcattaatgttttgcagcccaaaagttgttcacatttttcctatataagatctgt |
| | | ggagtgtgtgtttcaaagagagaactacagaaatgttaaagcaggaaaacctgaatgtgatgtgcac |
| | | attttcatcccacatggaca |
| 450 | 224989 | catttctgtcacatgcacttagttgacattacatctacatatattagctttttcctacatgagccatctatttacttagtaacca |
| | _at | gtgttcttaatgaagtatttagtcttgggtttcttgtaaaatttctctgcattccttagacagtgtactatacatgaaatattct |
| | | tgttgacctagtaatttatattattccatttaattcttaaacctatggcctttttattgagcacactcttaaatcattatttgg |
| | | cttgtaaacattcatctgaattgtggctacaatcctctttaaataatctaggaaaaaagaaagataaagcttacattttcacag |
| | | ttttggctcttaaacacattccacaaatgccattaagaatttattttgttttaggccagtcatggt |
| 451 | 224990 | gagcttctagaaatttcacttgcaagtttatttttgcttcctgtgttactgccattcctatttacagtatatttgagtgaatga |
| | _at | ttatatttttaaaaagttacatggggcttttttggttgtcctaaacttacaaacattccactcattctgtttgtaactgtgatt |
| | | ataatttttgtgataatttctggcctgattgaaggaaatttgagaggtctgcatttatatattttaaa |
| | | tagatttgataggtttttaaattgctttttttcataaggtatttataaagttatttggggttgtctgggattgtgtgaaagaaa |
| | | attagaaccacgctgtatttacatttaccttggtagtttatttgtggatggcagttttctgtagtttnggggact |
| | | gtggtagctcttggattgttttgcaaattacagctgaaatctgtgtcatggattaaactggcttatgtggcta |
| | | gaataggaagagagaaaaaatgaaatggttgtttactaattttatactcccatt |
| 452 | 225207 | ttgtgtgtaatttcatggtggcctagtgagtggtgcttctggtaatggtaatagaagctcaactatttttttgtggatttcagt |
| | _at | ttttatcatcagaagtcctagacagtgacatttcttaatggtgggagtccagctcatgcatttctgattatacaaaacag |
| | | tttgcagtaggttatttgtcatttcagttttttactgaaatttgagctaaacatttttacatgtaaatacttgtatttac |
| | | caaagatttaaatcagttgattaattaattaactcaaatactgtgaactatctntaaaacactagaaaaaagaaatgtta |
| | | gtatctcaattacaccaactgtgcaaatgaactttgataaaatagaaataatctacattggcctttgtgaaatctgggg |
| | | aagagctttaggattctagtagatggatactgaatactcaggcccacttaanttattaatgtatacattgtgtttttgt |
| | | ctttatgctatgtacag |
| 453 | 225242 | ggcgtgcaattttggtctgcgccacataaccattctgaagcttttaggcgttggagaggaagttgggggagtgttagaactgttc |
| | _s_a | ccaattaatgggagctctgttgttgagcgagaagacgtaccagcccatttggtgaaagacattcgtaactattttcaagtgagc |
| | | ccggagtacttctccatgcttctagtcggaaaagacggaaatgtcaaatcctggtatccttccccaatgtggtccatggtgat |
| | | tgtgtacgatttaattgattcgatgcaacttcggagacaggaaatggcgattcagcagtcactggggatgcgctgcccagaaga |
| | | tgagtatgcaggctatggttaccatagttaccaccaaggataccaggatggttaccaggatgactaccgtcatcatgagagtt |
| | | atcaccatggatacccttactgagcagnnannnnnnaccttagactcagccagtttcctctgcagctgctaaaact |
| | | acatgtggccagctcca |
| 454 | 225269 | aactgtgtacttgtctggtcagctgtgtatgatcagttatctacctcagagtctattttcttttgtgctgggacaggttgctggcc |
| | _s_at | ctccctgtttccacagaccaaatcctcctagctcaggagctagggctaagcagttatttctttcaagtattttttagttcttaaat |
| | | tttatgcttgtatttgatgatagatgtcagtgacatttcatagtttcaaaagtccttgc |
| | | tgctctgagaagtgtagattctagtgaaaattacatagtcataagagaaatgtgtttttgtttttgttatgtttcatttttttaaa |
| | | gttgtggtattattggttctatgctccctggaatattactgctttgtgaaagtccagactgaacgcagcaccctctgtgtacctag |
| | | tacagttataaacctgggtctctcactacttgatatttttgcattagttaagacagaaatttgatagctcggttagaggggagggg |
| | | aaatctgctgctagaaatgtctgaactaagtgccatactcgtctgggtaagatttgggaaacataacctctgtac |
| 455 | 225275 | gatgagagcaaaggggtcgcaccatatggaaatgttgaaaactattgtaaagtagtattatgaagtagcttttgtgtcattcatgt |
| | _at | cgatgacatgaaagtgaagtaaatttattctatgtaaattcacactaaaaccagtacagtaccataagtagaatacatgtaag |
| | | aatcacctagtcttcactatattgagtaaatataacatgctaatttta |
| | | caattaatgaaactaaacttttaaacatctccattatatctacatccttttgaaggtatttatcatagttgccaattttaatttt |
| | | aggattgactttctctttctgaatgacttcataaagtttggtgtgaattttgaagacttgggttactaatgattgtatctttgct |
| | | agtcaacaacttatgaaatatactcaatgcgtctgatgtgtcattaagtgcagaaataactaagacacaaataacctttgc |
| | | aaaccttcaagctgtgtaatattccaatgttgtttttttctttgtatatatacttatatcacgtaggatgtaaaaccagtat |
| | | gaccttgtctagtctccaaacttaa |
| 456 | 225353 | gccacacgtccaaaaccaatcaggtcaactcgggcggtgtgctgctgaggttgcaggtgggcgaggaggtgtggctggctgtca |
| | _s_at | atgactactacgacatggtgggcatccagggctctgacagcgtcttctccggcttcctgctcttccccgactagggcgggca |
| | | gatgcgctcgagncccacgggccttccacctccctcagcttcctgcatggacccaccttactggccagtctgcatccttgcc |
| | | tagaccattctccccaccagatggacttctcctccagggagcccaccctgacccacccccactgcaccccctccccatggg |
| | | ttctctccttcctntgaacttataggagtcactgcttgtgtggttcctgggacacttaaccaatgccttctggtactgcc |
| 457 | 225381 | gtatgtttgtaagtgcttctgcacgaatgtttatacatgactgtttccatagtacttatgtttttaaaaatattcagtcattt |
| | _at | cctactataatcctcatgtatccatgtaactgactcaaaaatacttcagccacagaaagctaaaactgagcaaatctcat |
| | | tcttcttttccatcccctttgcatgtggctggcatttagtaatgattaataatatggccagctg |
| | | aataacagaggtttgagacacaattctttctcaaaggagtcagctaagctgggtctacttatggacaaacatctaaatgtgtg |
| | | gaagtatctgatatttgacaatggtaaatttccacttagctagctagcattgtcagacttcaatctcctcatggctctggccg |
| | | tcctgttttaagcatgataattgttggccacatctcacatagttctcattgagtgagttcataaataaacagggttttt |
| | | ttttttaaagagcagccaagcacaaagtgtgactttgttgacattttatgtgactttgtcatatgttcctaacc |
| 458 | 225442 | agtgtcaggtgccttgcaggaaaataatctgagtcccaagctagcctgtgctcatccacaatcacaatgaacatgtcaaggaa |
| | _at | gaatttgcagagactcaagggaagcacaatgggataaggtaatcactttcagtgaaaaactgtttcttgaaaacaggcttg |
| | | gacacaattgaaagctggcttcctgcaaacacaccaagagtctgtaatctagcctatccattatatgtcctttattattca |
| | | tgatatcctattcttctaccttgttgcctggtaactattctgaggactgagtttctgcagcgatgtggtgcactcttcctg |
| | | tgatgaggaaacatctgggccccct |
| 459 | 225458 | ctggacacccggttaccaaagtcagcaaagaagatgcggtaatcgccgcctgatctccacatggtgaacacaacactcccaccaa |
| | _at | cacctccttgactggtcggtcttcagcaccgggggtgggcaggcaggtgttctgtgttgacgagaattgcacaggctaaacaca |
| | | aacacggaaccagagtgagaacacctcactcacggc |
| | | agcccaggctgctccctaccaggtgacggagcgcgccggggctgtgggtgccaggggctgagtgctagggactcgtcatgagtg |
| | | gggatccccacgttcctgtcactgctgtcaaacagaaggtaaacagtcttatgaatgtatttccttaggaaaacttg |
| | | taaaaacttttattaggatatctatttaatactgaactttggcctactttgtgatagactataaacaaattgaggaaatcact |
| | | atttctcacttctgtattttctcaaaaataattttgttacagagttcaatatactgtgtaccattgatcttctattgtg |
| 460 | 225575 | gaaactgcggatcccaaactgttccctttttcatttcttgaaatgttaccactacagacatttttttaaggtgaataaacagt |
| | _at | tgtgatgtgctgtacntaaaatcatgtttaatcgtataaggaaacatttcaatacacttatacaggaagaaaactatagatga |
| | | agtacatgtgtgtgattcagtctgattcacagaattctgagagtaatatggaataaaacaactccacttagatgataactgaa |
| | | gcatttcctgccttgtgaaaatttggattttaaattgctgttagaatgggaaatttggacactttatatcattgtataatttc |
| | | agaatttagtttctgtatcttttggaaaacatgattatagcaaaaacatagaaaataatctattactaaaacaccataaatg |
| | | ataaaactagtatgcttggctgttaactctaagatgttacttatgtctgttttta |
| 461 | 225602 | tgagggtgagcttactcagggcccccagaggaagccctcagcctctgccctccccccacacagggcgggagcccaggcctgt |
| | _at | tcctggcagctgtggctgcagctgtgctcctgctccctcctggaatgtgcgacaagcccaaatgttccngggnaggcggc |
| | | cggggcagggggcttagaagtgctaatatggttctgtgttttgcctgaaacgataccaggttcccctgaatagcaacttt |
| | | acaaggtccatgtgggagggaccaacccagatgccctgctgagtgtccctgaaaccatggcagctccatctgtcaagatg |
| | | gcaggggccggagtgagggggctgctggcttaacagcaggcatctgggcaggccagtcctcaaagcagctcctgaaggtc |
| | | tgtgttgcactgtcaccagtctcaagctatgcctctaatttcaccagggatattg |
| 462 | 225604 | gtgtgatgcatgtgagcgtctctggcacacacacttggacatacagttctgtgtgcgctcattcttattacaggagtgagc |
| | _s_at | aaaggaagcatttaccccgatggttacctagaccacgattatttggattggggggagg |
| 463 | 225626 | atccctgtattcatggcttgactttgtgactgctctacactgcatgtctgacattgcagagtgagctatgttgaggtaaac |
| | _at | tggttgtgttcattattttgcaatcagcctggtctctcccatgaagatgtcgtgtgcataagcacaatcatcactgatta |
| | | gaagatcacagcagaatacccttggattagagagaagttcgtaccttgcatttctctga |
| | | attctagtctctcataagcactgctttgctggatgattttcactgctttgtgttaatgactttgagcgatctctcacatg |
| | | atggggttctttagtacatggtaacagccatgtcatcttacacacctagcattgtgaatgctgtagtgacatccttta |
| | | taggcaccttacagctcaaaacttttgtttcatttcatgccttacttatca |
| 464 | 225688 | agtgccatttattctagtttatcatgttttgcatgtttgaaagtatgaatgtgctctttcctaaaacatggcaaatgaat |
| | _s_a | agatgtagagaataacaatattacttacaagatgaaatgattagattagaagtgtccctttattaaactttgtcag |
| | | cctgactgggtacaattcttttgttaatttgcagtgtggtttgtatacacgtatacgtgttatcaataata |
| | | agattttgcaactggatgacacaagattttacttgaacagtgaaggacaaaaatcatgattgtggaagatatttttaa |
| | | aatctgattttgcagcgatcacttttaaaccctgtagtgatgtaagactaaaatataattgctaagattttgttggt |
| | | taatgtaaagatatgacttttctgcactgtactctcttcataggattgtaaaggtgttctaatccaatt |
| | | gcatgatgtagtaagcctcttaaatatgtgtgtta |
| 465 | 225710 | tcctatgtcttctttcttaaatccagttgctgattttgtaaaatacagttgtgataaagcagcattacgggggggaa |
| | _at | aaagctatattccaactggtgttaaatgtattcaacaaaatcttacatcatacagtatttatttcttaattaata |
| | | gaacttcagtgatatacttggtagatatctcaagccttttgtcttttacacaatggtgctctatcctattgttttct |
| | | tttcaaagaagcatctgaacacttgcatttctattttcctatccaaaggcatccacatctaagtgtgtttttaaagt |
| | | tgattaaaattatttttctgttaaagcattctgaaagtgtttgtctttacctagaatgatttgtacacactcgtggt |
| | | caactgaacatgaatgtcagtagtagtctaattatgggaagggtaaacgtgttagattaaggctcttaaagctctaa |
| | | accatataaactatggacttgtatcatgatttaactgttcttagatctttcttacacagtgattcattcctctatt |
| | | tgtacagtggcttt |
| 466 | 225720 | tatcaaaggtttgccagccaataaagtgcatcccaagtatacaggggagaaagctagactcctacagggtcctagagtttaagtaa |
| | _at | tttttttgttattaatataggtaataatattctaatttttattttttggttccaaatgtaaagctccttgtgtttacctct |
| | | gtttatgtcattcttgacatgtttatctaaattatgtgtgctctgtgacaggtgaaatgtaa |
| | | atctgggatccatagtcaagatatcataaggacctacttcccagcctacctttcttcctctacctgataatgataatactcaaaat |
| | | aacaacattcaaaggaaacacaaagaaatcctgctttcacatctcctatttcttgggctccttaataactactgatggtttgt |
| | | tcatgaaaaaaaatttttaaatcaaaagattgtacttggccctgagttgaaaaaatttcaaaaatcaaaagtttgtacttggc |
| | | cctgagttgaa |
| 467 | 225721 | cagaaacatgaccctcgctggtcttgggtccacatatcattggactctgggggacacaaagatgcctgtgacactttggtgttgc |
| | _at | cgagttagtcaacaattattctgggaaaaagcagaattgaattcttctctagatgtcctaccagggttggccaagggccacaaa |
| | | gcaggctaataaattcccacaggatccagacaccaggcaaaattgctctaagaagccagttactgtcatccctctatggttcta |
| | | gaaaaaatagtacaaaaatgacaggtcatcctatgagcgtcatgccaatgaaaccccatcttctggagaagcccttgaatc |
| | | agaattatcttttttcttgatgtcgtcagatgcagccagtttcttaatttttttaaaaactgtatgtttctgtggtatgtata |
| | | tttgtacacctaactacctggcacttggaaatcacagcactact |
| 468 | 225728 | ttatcctgcgacaacactttcagcagantagcctctccttatctcacagatcacaagcacccctagatagtgtgattctgtcaga |
| | _at | tagcatttatgcaaaaatctatgaagttaaaagatcgtagaagccaaatgaaatgtacatatctactgactgatgacaaggg |
| | | aatttcattaggaagaaggtaaagaaacatcgttgagtagcctaccttgatttctgtcaagttcataaccagcttcatatttt |
| | | aaaggcttcaggtttgaaattaagtcaactgcatgcagctttgctgataaatgaataattctctttgatgccatttat |
| | | gagaaaagacttcaatatctgttgcctgtcatatttaagaaaaattactgtttctactctctgtatctgattttaaaa |
| | | gaaaaaactattcatacctggcttccaggtaattgactttga |
| 469 | 225782 | gaactgagtcaatatggcaaggtgtatgtgatctgtgggagttatgccatttaacataggaagtgcatgggactttccctctct |
| | _at | gcactccagctcttactgtaccattagaagatgcagaattctgttggtgtgcaaaaagtatagccttacattcaagcaga |
| | | atggatctgaagaaagcagcaatatctgttactagagaacattcccatgtgtttaaactcttcacttcttagatgca |
| | | tttaaattcttaatgcaaatgacgtagcaatttgaaaacttctccgtattacttgtgaaaatgtcttgctttaaatacaa |
| | | aacaaatggtaaaggggattatcttttgtttagatggttaaatattatttttgccttagatagctttgtaataattttt |
| | | ctccagacagttcaacacttttga |
| 470 | 225894 | tactgactgactacaggggctgattgtgaagcacgaggaaccccatgtgtgtggagactgtagggtgagagcacacaattatt |
| | _at | agcatcatttctgagtgatctcacagattttttttcttgtgtttgttttgctttttgacaactgcttctcccacgttcc |
| | | ttgcaattctattctctcaccttcactttactatttgtattcgatggaccaggataattcaggc |
| | | aaggttaccttgtaaacttnaattggccacacaccatgttgtcacccagctggctatgaagtgaataatggtactgaaagta |
| | | aacctgaagacctttctcagatctattttaagtctgagtctgaccaaccatggaaaatattcgacatgaattaatgt |
| | | agagaactataaagcatttatgacagctccaagaaaaatcatctactctatgcaggagatatgtttagagacctctcagaa |
| | | aaacttgcctggtttgagggtacacagtaccattttaatcttctgaaaatatctgtattcctgctctttttctgctgtcac |
| | | tgtcaatctgctatattttt |
| 471 | 225895 | ttcctgacgggaatgttgtgctataatgaatctgcataacgcttgggattctaggaggaaggaaggttccatggacatgtaagta |
| | _at | cagcatattcccctcagtcttctaggagggcagagtgaatcccagaactggtaagattgggaatctgagcattgccacttta |
| | | atcttagaatatttatcattttgacacatcctgttttttagagaggaaaac |
| | | aaacacagtttctgcattggtagtgtaaagcataccttgttaggaacgtgttttgtaagacacatttgggttgtcattctaga |
| | | gcatgtcaaactttgtacttcaaaatatatttagtatgattgttagtggtaacatatatcaaggctttgaattaactgtt |
| | | ttatttaattttcacaagaagcacttattttagccataggaaaaccaatctgagctacaaatagttctttaa |
| | | aataagcccaggttatttagctattctagaaagtgccgacttctttcaagaag |
| 472 | 226001 | ttaaatattcattccattacatctagactcaccaagaactacatgttatgatgttaagttgaagttgaaacatgatgttttg |
| | _at | cattaaatttaagatatgcaaatttatgtagagaaaataaatgttatataccctataatctttcacctaattagtatttaa |
| | | ttatatggatttgttttatattataaaagatgttttgattttgtcttttgatattgacaaaattgtttg |
| | | gatatccttatgttctcaagtctgtatctgcctcccctgccttatttcttatgattgccacagttaacccattgtgcttct |
| | | ttgtaatcaaacagtttgtgggagaatgggcttattgaatgtctaaaaaataagtttaaagtgtttgttaccctaagtttt |
| | | ttacatttttaaactctaattacatatgtgaatgttattactctcagtgaattgttattgtttgcaaaaat |
| | | gcactgggcagtaacattttgt |
| 473 | 226051 | gccggactggaaccgtctgagcggcctaacccgcgcccgggtagagacctgcgggggatgacagctgaaccgcctaaaggaggtg |
| | _at | aaggctttcgtcacgcaggacattccattctatcacaacctggtgatgaaacacctccctggggccgaccctgagctcgtgct |
| | | gctgggccgccgctacgaggaactagagcgcatcccactcagtgaaatgacccgcgaagagatcaatgcgctagtgcagga |
| | | gctcggcttctancgcaaggcngcgcccgacgcgcaggtgccccccgagtacgtgtgggcgcccgcgaagcccccaga |
| | | ggaaacttcggaccacgctgacctgtaggtccgggggcgcggcggagctgggacctacctgcctgagtcctggagacag |
| | | aatgaagcgctcagcatccc |
| $47 | 226084 | aatggcttctatgatcagaactgggaaaacagtgnatcttatggtggaagaggtnctcagcaagtgtacagtatttaccttcct |
| 4.0 | _at | ttgtcttacatnggctttttaaattttccattaatttcaacataattatgggaacaagtgtacagaagaatttttttttta |
| 0 | | agatatgtgagaacttttcatagatgaactttttaacaaatgttttcatttacaggaaatt |
| | | gcaaagaaaattctcaagtgatagtctttttttttaagtgtttcgtaagacaaaaattgaataatgttttttgaagttctggca |
| | | agattgaagtctgatattgcagtaatgatatttattaaaaacccataactaccaggaataatgatacctcccacccct |
| | | tgattcccataacataaaagtgctacttgagagtgggggagaatggcatggtaggctacttttcagggccttgacaagtaca |
| | | tcacccagtggtatcctacatacttctttcaagatcttcaaccatgaggtaaaagagccaagttcaaagaaccctagcaca |
| | | aatttgctttgg |
| 475 | 226103 | ggaacctatcaatttgagtggactttttctttagtagtacaccatttnggttgtngtagtttcaaagtctttctgaagcagatat |
| | _at | attgggattggagcggggtggggaaaactgtcactcctttcagaggaaaaggggaggagcatggagaaaaacaaaa |
| | | attaaaggacttaaagaatggctatacagtgttgagtgttgaggatattaaacatgttatttttcaaacgtatgtaat |
| | | atatattaaatttataaagcaaatttatgttgtgatcttgcctgaacaaattatattttaatgaaaaaactttctattaatagt |
| | | tcacgcaagagaaaacactttcaacatagtcgaaggcttcaagatctaagtgtatcagacttagggaaaaagtggcacaacctt |
| | | cgatttaaaattctagtctttaaaatgagtttgtaaataattagctattacgttctattaagttgtt |
| 476 | 226147 | agggatctctctacaagagcccctgcccctctgttggaggcacagttttagaataaggaggaggagggagaagagaaaatgtaa |
| | _s_at | aggagggagatctttcccaggccgcaccatttctgtcactcacatggacccaagataaaagaatggccaaaccctcacaacccc |
| | | tgatgtttgaagagttccaagttgaagggaaacaaagaagtgtttgatggtgccagagaggggctgctctccagaaa |
| | | gctaaaatttaatttcttttttcctctgagttctgtacttcaaccagcctacaagctggcacttgctaacaaatcag |
| 477 | 226302 | ggtaattatggaaactcctcaaagaggagaaagtaatttttttccagacatttttctcattctgtgtctttcacacactagtt |
| | _at | tccatagttcgagaattctgttttttaccattgggctgtgaatgttcacaatatcagtcctgttgaattcctatga |
| | | ggtaatcacaatgtgtatatgttcattttctaggtatgataaaagaatgtatggctttttattctgtggaa |
| | | gtaaaatcctgaacgtttacaacttttccttaacttgtaaataaaaaattgtaagttttttctttttttacagaaaac |
| | | ttagcttgtgtaattctgttagtttcagatttctctcctgtttttgcaaattgtgggaaagattgacaatgcaaatgtgtc |
| | | aaagacatactgttgggtgc |
| 478 | 226303 | acatgggtttatacaagttcctcttgagaaggcaaaaagaccaccatgtgtgagagctctttgacttggccaataggggcctatc |
| | _at | ttaatgcacttgtttggacacatttctgatcttatttgtaaaggctgcaaaaggagaggatgaaatgctgtaaaagtaggaaa |
| | | tgaagtggaagctggaagaaaatgtaattggtggtacagctatgg |
| | | gccagatggtggaggggagggtggggacccctgccggcaagcagagtgtcacagctggctttcctcacttgggaaaagggtact |
| | | gccggtctagcagcctcctctgtactcagccaggacacccagcgcgtgggacctgtttgtgtctgttttgcttccttgg |
| | | gaacggcacagtcactcaccctgccatttgcggaaatgacctggtgcactttgactgttaagcaatgcgttattgctgtagtc |
| | | aaggttagtgcaagcaaggaaacattcccagtaaggtatttgtttccattttctgtctgtgcttctgtcagaaacttg |
| | | ctaggac |
| 479 | 226304 | caaacgtcctggactgcacagacctcccactccagaccatccaggcctggttcccaagacccgatccttcccctgcaaccagaca |
| | _at | gtctacaactgccccctccagcccattttctgccgtgaaaccccagccagccacaccagactctggaaccctttttcg |
| | | actgccccaactcttggacaccaggccaactagaacacccaacac |
| | | caaactgtacagactctcccaccccaacctccccagactctgcacggatgtcctaggccccctccccaactctaaccagacccca |
| | | tccccctaagtccctttgtcttgacccccaagtcttcaaccagatatcctcggcaacccacctcccaccctc |
| | | ctcctcttctccttcaagacccaactgagcacccgctctgattccccacagcctttct |
| | | ccctgccaccactcccttagtctttcccaggcttactctcccaataaatgtgctag |
| 480 | 226333 | ggtggagttggtggtcttaatttggagatgcaggggcaacctgtgaccctttgaggcaagagccctgcacccagctgtcccgtg |
| | _at | cagccgtgggcaggggctgcacacggaggggcaggcgggccagttcagggtccgtgccaggccctcctcagtgccctgt |
| | | gaaggcctcctgtcctccgtgcggctgggcaccagcaccagggagtttctatggcaaccttagtgattattaaggaacactgtc |
| | | agttttatgaacatatgctcaaatgaaattctactttaggaggaaaggattggaacagcatgtcacaaggctgttaatt |
| | | aacagagagaccttattggatggagatcacatctgttaaatagaatacctcaactctacgttgtttcttggagataaataata |
| | | gtttcaagtttttgtttgtttgttttacctaattacctgaaagcaaataccaaaggctgatgtctgtatatggggcaaagggt |
| 481 | 226430 | atttttccatcttattcaagtcagagcacttttttttaaatagcaatacaacaggcaagagaatactcaaaaatatttaaaac |
| | _at | tgtattgataccaagagtatgttttaaatattttctaataaatacttgagcggtttttgtctggcaggcttccaaatttg |
| | | ccaaaattaagcgttcagtattttcaacacatacgctttttactggtttatactgaactatctg |
| | | atgagaattcctgtgttcccaaagcaactgatgtttacaggtcttgtgtttctcctcctcctttctaaggatgagggaatcca |
| | | caacagactttctctagaaaacactaatgatggacaactttttggtgtcatcaatgagttggctactaccttgatg |
| | | taaaaatttgtaaggaaaattttcaccatttcgagtgtcaagtgtatttttaactgtctggtttgta |
| | | cttttatgacttttgtactaccaaagcgga |
| 482 | 226492 | agtgatctctggaagcgctaaagctaaaatttctgttcttgaaacacttcagctttgcaactaaaatattacagattaataa |
| | _at | taaattaaaccaaccaatgataaacactactcagtccaccaacaacaaacgtgtttgaattcaccttaccaatatta |
| | | atcccagcgtgtgtaaaacagaacagtaactctatgtgaccccagataacattttgtaacattgtgcttccttgtag |
| | | tttgtaatgtgagttcaatcagtatttatgttgaaatttctaacattaaatctagtctctatcctgttaatttaattttt |
| | | aaatgctttatccatttgtgcaaaggtaaacgcagattgtatcttttttaatggtacggcataaaaagtaaccctcaagtg |
| | | aagtgtctcta |
| 483 | 226594 | tgacgagacctcttcaaaaacccacagtaaaactcccctccctccagttggccaccagtctgccaccaaacatgaacaaattctg |
| _at | | ctgctaatcggtttcccttgtgatctggttcctgaggtcttcggatctgtgcaatgaattatttattgttttattaaaccga |
| | | cagtggtgtcccagagaggaaccataaataaaatggaaatctggtgctgt |
| | | gataaagtaataactagcattaatgagacctggttttcctttcagaaagtccagtatacctgtaacaaaggttaaagcaatttat |
| | | atttaatttgcattctgatgttaacatttaaacagcaattctaacaaaaatgcatcgagtctaattcttacctct |
| | | atcaaaaaacaactgtataaatttatgaccaacattaaaacaaaaacaaaatgtaaatt |
| | | ttctctttagaaatgattaactggaaatgagtgagacagcaccttgtgggttt |
| 484 | 226654 | tgctcgcattgatcatcctaatcatcttattcagcctatcccagagaaaacggcacagggaacagtatgatgtgcctcaagagt |
| | _at | ggcgaaaggaaggcacncctggcatcttccagaagacggccatcngnnnagaccagaatctgagggagagcag |
| | | attcggccttgagaacgcctacaacaacttccggcccaccctggagac |
| | | tgttgactctggcacagagctccacatccagaggccggagatggtagcatccactgtgtgagccaacgggggcctcccaccc |
| | | tcatctagctctgttcaggagagctgcaaacacagagcccaccacaagcctccggggcgggt |
| | | caagaggagaccgaagtcaggccctgaagccggtcctgctctgagctgacagacttggcc |
| | | agtcccctgcctgtgctcctgctggggaaggctgggggctgtaagcctctc |
| 485 | 226682 | gctgattctaattgtgtgtaggtcttgaggattaagcacacaaatttcacaaacttctgtttgagtaaacaaactcagcctt |
| | _at | ctgtaaatatacatgcaagtttggaaacagtaatactgtacctataaatatatgctgtctgntttgtgtacagtatgt |
| | | aaaaactccttttctgccacactaaaaatgcaagccatttatgggaatcctaaaactagta |
| | | ttgaactaaaactttgctaatgatctttattagaggatcgtccaacttttcacttaccttgggttttcttttcaattcact |
| | | cttacactagtctgcttatttccagctgtttattttattgagtcctgaatttaaaaaaaaaatattttgattcattttgt |
| | | aaatacaagctgtacaaaaaagagagatttaatgttgtcttttaaatactccaattttcattctaatatgaa |
| | | tgttgttatattgtacttagaaactgtacctttaatattacattacctttattaaaagtgcattgaacacatcaatttta |
| | | gatgtgctttatgtactgttatccta |
| 486 | 226694 | taagcattcagtgagctgccaattttgattttgtgttgctctttacccaaattattttttctttgtttttctttttttggg |
| | _at | ggaggaggggaaaaaagcagcaatactgtgtttggaaattatactctgtatctggttttcctgtgtatgttaaccact |
| | | taaatgttattatcctgctttggttttagagtgattgtgaggcattcaatgca |
| 487 | 226811 | agtgcctaaatcttgtttacctatcactttaaaaaaataattgaagtgtaagctaaataaaatgcttggagtttngcctgg |
| | _at | gctagtgagagttggtgcaaattcttgtgtgtgtttgcataggaaggtgagatgaccatctactaaagaggaagtagct |
| | | aaatacagatctgtgggtgtttttaaaaaaactcaacctatctggtgttttattttaatggataaaaatgtaatttttc |
| | | taaggtagcaacttatttccaaattaatatagatgaaaaatagataccaattagactaaattgaaagctttttgttctat |
| | | atttgcatagcctttgaaatatttcttagtgcctaggaggtctggggattcctctttcgtggtggtcactaaccttactt |
| | | gatgcaga |
| 488 | 226818 | aacaccaattatgcaaactacttttttttccagcagaaagggagctgacatgatcaaatccatgttttcaaatgaactgaa |
| | _at | aaaggcatccagcaccacttatancacatttatttcagttccaggctgacagccntggggnatctagcaggatnna |
| | | taattgtcatctggtgagagttgggactttgctcaattttaataccaatctctcctgatt |
| | | gtaattacctccactactttcatgatccccctacaatatttttttaaatgatatatttattcactgaatcaaatgtcatta |
| | | atgagttatcttctgtggaggatgactgttctctttgttaatgttcacaatcaagatcttgggctgagaagaggc |
| | | ccttcacccaaggagtttgaagtatcacagcgtgtgggaaggtgggaaccaggatacccattcatttccaacc |
| | | gagacacagagaagtgagtcacagaatttgagccngctctcttgactgcccagccagagacactgatttct |
| | | gtaacctcttcacttgatcctgcctcttaagcatta |
| 489 | 226834 | tcatttctagagtacctgagccaaacaaatacacaacggaagctgcagctgtatcatcactagcaatttgctcatcattattta |
| | _at | ctacctttgaacctaaggtttcctgcctatgcttttgaaagcaaaaatcagtctcctttgcatgaaaaagag |
| | | ccttagatttttaaacatgttagttaccagaatgctaaaataccagttgattacccaaattattt |
| | | tggaaatctatccataatggaagtctacaacaaacacataaaacagattacactaagagctgagaaattcaaaggaactgaa |
| | | gattctgagagataaactgttcaagtcttagcaatgatactgcacttctctttgacaggtt |
| | | ctgggcttaagttagaggccctactggttccaaaccatattccactgactttgcaag |
| 490 | 226841 | cctctgagtgttttgtgggttctgggtgttttgtacattttagccaagctaaccacttgtctgcaagtactgactttcct |
| | _at | atgaattctttgaagattattgagtcagaaaggaaaaatatagccccaaattcccaggcttttaatgcattacattaact |
| | | gcctattgaaatgagaagttcttcacaaacttgtatacccactaacaagattgcacataaac |
| | | atgcattaaagtatatactaagaaaccctctgtccaacggctcatgcatatgaagtccgaacatgggagtttgccaattg |
| | | cattcatcaagtcgttttgcggagtcagatccctgatggaagagctcacaggctctgccttccaagtcctgggtt |
| | | cctaactggtgaccttagcctggggtctgtggggagaccaaccctggcttccaagaaaaccacattc |
| | | catggactatcagaaatagacacagatttgggtgacaaagctggctctgtatttgcatttt |
| 491 | 227006 | gcaagcgcgtgctgagcaagctgcagtctccatcgcgggcccgcgggncagggggcagtcccggggggctgcagaagcgg |
| | _at | cacncnngcgtcaccgtcaagtatgaccggcggganctgcagcggcggctggacgtggagaagtggatcgacgggcgcct |
| | | ggaggagctgtaccgcggcatggaggcagacatgcccgatgagatcaacattgatgaattgttggagttagagagtgaa |
| | | gaggagagaagccggaaaatccagggactcctgaagtcatgtgggaaacctgtcgaggactt |
| | | catccaggagctgctggcaaagcttcaaggcctccacaggcagcccngcctccgccagccaagcccctcccacgacggc |
| | | agcctcagccccctccaggaccgggcccggactgctcacccctgaccctcttgcactctccctgc |
| | | cccccggacgccgcccagcttgcttgtgtataagttgtatttaatggttctg |
| 492 | 227052 | cactgttccatggtcagcaagtcatatttcataatgtggattttccaaaataattattgaatacagctattctatggctactttt |
| | _at | agtgtttttgtggtatgtggtgtgggagtgtttatggaattaccagtatcttaaattttcaaaggaaccttggaa |
| | | gtctatcactctaaatgaaagtctgtcactctacatgaattatgtgctcaaatttgaccaactcagtt |
| | | taagacacaaaacagtaatttgaagaaggaaaaatgaagagagtttctagtttaatgggttaaatttttgttgttgcaatagta |
| | | agtttagtcttcttataatatttctaaatgaaaaatcataggtatttgttaccatgtgtgaagattantttgt |
| | | taaaagcaaaagtggtcgtgtgatatgctaaatgttaattactgattttatatgtttaaatcacgccaaacaaatta |
| | | tgtctgtgccatccagggtctgttgttaatctttttctgagtacttggattgggataaagggcttgtactatgcactt |
| 493 | 227061 | acacttatagtctactgcccatgtaaggatcagctccggctaagaggccaaagatgggtgacatcgttatgctctgccnttta |
| | _at | ttttttctttcttacccacttagcttcctaattggaggaaggaggcgtggtaaaggtatatgaagactatggtttaat |
| | | tagaccagaaaacactgtcataatctctggggtcatcagaatgtccagttttgtctttg |
| | | ggccaagataagggcagtgggatttatgatgtgttgtttatagtctgaaactactctggtgatcanccagggtcagtttct |
| | | ttaatgatggtttccaactggcctaatacattaagtaagactggctgataacatgaccagacagacataaagacc |
| | | ctgttgggaatgacattgaactctcaaagtcaagatttcttacacaaatctatcagctgg |
| | | agaaaatgaaggcagtgtggtatatgtgtgcaaataaggacattatgaagcttaaatatggaatgtctcttggaccccc |
| | | gatgtcatctgtattctctttttcttcttgtactatatcctttgcctgtaaata |
| 494 | 227099 | tcccagtgcgaactctgctgtgagtgtgtgcggggaggcgcgcccgcgctgagtcggcggcgggtagccactccatgcccttgtcc |
| | _s_at | gatggtttgcaactccgattttgcacaccgctccaccgtgccccccagcgcacacccattcacactcacgccaacactctcgc |
| | | tgaacacttttataattgttaggcgtggccgttgggactttgggcgcagcgcggctgctactgcgtctggaggattgatattta |
| | | tttttgcattgcgatggctgaaggcatttatttaacgatctttttacctggatatgtctgtgaggctcctgaa |
| 495 | 227235 | gtgaagtaccatagaccatattgtctagatgggacaaaataataaattttgactaagaacttactgtttagagagaaaaaata |
| | _at | gtaaataatgttcaaaatgagaagacaataactgacaagtctgtgatttttgggttagatgagaccacttt |
| | | tgatttttgaggaataaaaggagttagttttttggtcctgatgtgtaaaatgattcatggggttt |
| | | cacatacagacaagtagttttcttcagcaaatatttaacatatctctcaatatatttttcaccagtttttggtaaatatgcat |
| | | tgctataggtataccaagatttgttatgtttagtaactaatttcattattctgtatatgtaaatatagctattggaccct |
| | | gcattgaaaaccttcttagtactaacaacactccttttattattaccttcatagttactttaaatgacatatttatttcg |
| | | agttttacttctggctgagttgcaaagtctatgagaatgttacctcctgtctcagaagcttgttagttattgtgccacc |
| 496 | 227265 | acatctgctagaaccttttgccttaactattcaccaatatatgctaatattcataaatatggattgactgtttacaaacattaga |
| | _at | atcttgtcttggttccattttgatggctaatatttgttatcttaattaagactatttctgaggtcatgattacttgaaa |
| | | atattgactaaaactgggtccttagaaattccaggtggagctgatttacctatgactgagggg |
| | | aaaaaaaaatcaaattttactgataatagtaatgctccaaatgaattaatgacacatctgttcaataaataaagagcttaaatat |
| | | acaaaacataagaaatctgggcaacaaaacttgtggtctttacttttgaatagctacccaagaaaaggttttaaaggtaaaag |
| | | ttatgagtaatgtcatcacaataagctcttgtttaaaattcttttcttttatgtataattaggtttatgtttcatgtcttt |
| 497 | 227404 | atcccatgggcaataaagcgcattcaatgtgtttataagccaaacagtcactttgtttaagcaaacacaagtacaaagtaaaat |
| | _s_at | agaaccacaaaataatgaactgcatgttcataacatacaaaaatcgccgcctactcagtaggtaactacaacattccaactcct |
| | | gaatatatttataaatttacattttcagttaaaaaaatagacttttgagagttcagattttgttttagattttgttttcttaca |
| | | ttctggagaaccgaagctcagctcagccctcttccttattttgctcccaaagcctcccccaaatcatcactccctgcccccc |
| | | ttaaggctagaggtgagcatgtccctcacaattgcacatgtcaagccatcagcaaggcgcatcacacaaaaggcaccaag |
| 498 | 227522 | cagtgagtcgaggttgcgctactgcactccagcctggacaacagagggagactctgtctcnaaaaaaaaaaaaacctacagct |
| | _at | gttcaaggaccagctgacaggtcaagtgtggccttttctggtctttgaacacatcatagaaagtgacaaatgctgcaaagc |
| | | catgaagaacatgaactataaacgggtagactaactgcccagcttagacacttatctatgccacaaaacagctgaatttgtc |
| | | acatttatatattgcaatatgggaagtattgagatcaaaacaggattccattgacctaattat |
| 499 | 227529 | gccatcacagttgcgattccatgagtagctgctttatgactgctttttgtactatctggatgtgcccagagttacttctgt |
| | _s_at | acaagctctgtatctatgtccgttgagaacattattttaacaagaagaacaccaacagtagcatgaaatataatactgttt |
| | | tataattctaaagctgctgttaatttatgaagtacataataatctaatgtaaactgcagaagtcagagcaa |
| 500 | 227561 | gcaaccattggggaatgaccttttcatttcagaagtggatgaggaaggtggtgtgagcatcaggtatattactggaccatt |
| | _at | tcaagtgctggtgagaagaaaggaactctttgcctgaactgggcttggttttccaagtgctgctttggaaatgaa |
| | | gacccagagatgcagagcttatggtagttcataaatcttcatgttctattatctttcatctg |
| | | ccaataaagttcattttcaataatgtccaccattgctgtgcccagaataaccacaggcaaacatcaaaacaatacgcataa |
| | | gttagacaagattaaatcttgtctgatatctgcacaaacagatatgcaccatgttggaaac |
| | | atgtgttttcctagtcccatccaggcttcccacaagaaagccatgatgtgggtctaaacc |
| 501 | 227623 | attgtttcccatagcagaatgtcaatattcacagtacatttctgtaaagagcaaaccaatataatgttttgagtgttgaaaaaa |
| | _at | attccagatttntgaagaattagacaactcttcatctaccttatttctagttcacacagttatctcaaattccactga |
| | | aactaatgggatactgtcttgtgtagatgccagttgagtttataatgtgacctagtaaagctgtcttttttgttgtgtt |
| | | gtatgagtgtcggatcatgcttttaggaatacttttattaaaatggtgtgcattcatgcaaaaggccaactggcttttgt |
| | | gaacaatagatcttttctcccctttattttgttctcttgacacttttgtgaaaattacctagcctgata |
| 502 | 227662 | aaagccttccctaaaaagagagcatgccagtcatagagacactaatttggcacttttcctccttcttaggtttaaatgttata |
| | _at | ccaatgcatgtgtttgaaatatccattaagatgatttaaaatttgtcttgtattttgagtttcttaaatgcgtggatt |
| | | cttgttcatttatctctgaatatgtctcttttatattttggcacaattatacacattggaaagggcca |
| | | acctattagggctcaagtatgtatatgcnaaaaaaaaaagttatatcaaacaggcacagttattacaactagagagaaatt |
| | | ccagaaatatttgtttttttaagagaaagtaaattttcacattagatttctattcaaagtactaatatctacatgg |
| | | tccacacttttctattttaaaaaattgtgttctcttgttaaatagattaacatttccacttctgtttatcacaa |
| 503 | 227682 | atctttccatattgaggacagcctctgcatataaaaatgttcattgggagaaaaattatttttaaaactaaatttttatagtag |
| | _at | tcctccattactggcaattaaggagcaacccacaaaatgtcattatgtggttctttgattaggcataaaaagtattgcaaacag |
| | | cttttctattcctttggaatatgaatctttttaatgtctaaagttaacctgcaaca |
| | | ttttctagtaggacaagtcaatgtaaattaggtaatttttgcctgttttataccttctgtngcattttgttcaaatgaaaaca |
| | | ttttttaaaacatgattgaatttttacttctcagataattggtttgagtgtggaatttcttttttaacactttgatatgt |
| | | aagttagtgtaaattaatacttttataacactactaggctttatantatggaatgttttaaaatttgaaatt |
| | | tttgaantgttaatttagcagtactttatcattatgtgccaatactgacaag |
| 504 | 227705 | tttactccagtcattcgatgttgctgagatttacatatgactcttgtcaacatctcatcttttgacccaatcttattcattta |
| | _at | ataagaggtctcattcatttgcatggaaaaatgctcattgtatattgcaaagtgaaaataacgagttgcaaaacagtgtat |
| | | acatatatgtgtgtatatatgtacactttatttgtacatttctatgtgacataatgcaaagg |
| | | aaagtgtctgattttattatacaccaaaggttaacagtgaatctctgtgtgatctctttttttctttttgcctatctgcatct |
| | | tctcacttgccaaaaaatgaatatatgtttatgtgtgtatattacttgtgtcacaaaaaaccctaaagtagacagtaa |
| | | aagaacttgtcaatcgcctttggaaggcaatgaaacacttaataaactctcaataa |
| 505 | 227725 | agccaagaactgaccggggccagggnctgccatggtctccttgcctgctccaaggcacaggatacagtgggaatcttgagact |
| | _at | ctttggccatttcccatggctcagactaagctccaagcccttcangagttccaagggaacacttgaaccatggac |
| | | aagactctctcaagatggcaaatggctaattgaggttctgaagttcttcagt |
| | | acattgctgtaggtcctgaggccagggatttttaattaaatggggtgatgggtggccaataccacaattcctgctgaaaaaca |
| | | ctcttccagtccaaaagcttcttgatacagaaaaaagagcctggatttacagaaacatatagatctggtttgaattccag |
| | | atcgagtttacagttgtgaaatcttgaaggtattacttaacttcactacagattgtctagaagacctttcta |
| | | ggagttatctgattctagaagggtctatacttgtccttgtctttaagctatttgacaactctacgtgtt |
| 506 | 227727 | gggctgttccagagattcgatcctcttaaggcattatcagtgagcaaatgtgaaggaaatggtgtctggaagaaagttctg |
| | _at | gttcacatgccttgtagctaagtctttctgcaaacaacctcccttccccccgtcgagtcatttggtgactttgatgggggg |
| | | atttctggttatgtcaaggctctggagacaggaanggcctttggccgccttgggtagttgacctgccttttctgactccgg |
| | | gacgagccagtcctaggctgcctcngggagcacttgaggtatcccgcaggccatgaggacccactgggcagctcctgg |
| | | acagcctcttggctccagcccccacccgaaagtggnacactgnntccgccctggccacctggggactggcactgtggt |
| | | gcacagtggcccaatgtggccaacggaagtttt |
| 507 | 227735 | cacaggtgtagcactcccaaagcaagactccaganngnnnnnnacctcatgcctggcacctgaggtacccagcagcctcctgtc |
| | _s_at | tcccctttcagccttcacagcagtgagctgcaatgttggagggcttcatctcgggctgcaaggaccctggga |
| | | aagttccagaactccacgtccttgtctcaattgtgccatcaactttcagagctatcatgagccaacctc |
| 508 | 227736 | caccatgaccggtcacagctacaaatccagagaccatcaatcctgctagagtgcagggnggcaagcacccaagggtggctgac |
| | _ at | caagactgcagagtctcctccatcttcaggtccattcagcctcctggcatttaactaccagcatccagtggtccccaaggaat |
| | | cccttcctagcctcctgacatgagtctgctggaaagagcatccaaacaaacaagtaat |
| 509 | 227826 | ggttagattccgttttgactgcttggtatctcattgtaagcatttcccagggacactacacagtctttataattatgatcact |
| | _s_at | acatcttttggataacttaggcttttgcactattttgtcacgatgcctcaaacttttctatcattgtctgtaattactcctat |
| | | aatcttttatt |
| 510 | 227827 | aatatctcaagatttctaggcattttcctatgtctaatagtaggatgttctctctgaatttatttattacaaaaataacattac |
| | _at | aacaataaagcaaaataatatttaaaagaaagaaaaaacaaacagtcgcaataccagaacaccattgtagtcgtttgtgta |
| | | tgtgtgacttctttacttgtttcatgcagagacaagctttgtaatacctttgtaact |
| | | actgtacaggtgcgtggttagattccgttttgactgcttggtatctcattgtaagcatttcccagggacactacacagtcttta |
| | | taattatgatcactacatcttttggataacttaggcttttgcactattttgtcacgatgcctcaaacttttctat |
| | | cattgtctgtaattactcctataatcttttattataaaatattgaaacaaagtcacatcactaaaaaca |
| | | ctgtgagtataatttattagtaagtgtagactgtgtatcattagcaaa |
| 511 | 228133 | ggagctgcttcaagaagaaacccggcagaagctcaacgtgtctacgaagctgcgccagctggaggaggagcggaacagcctgca |
| | _s_at | agaccagctggacgaggagatggaggccaagcagaacctggagcgccacatctccactctcaacatccaggtgcctgcccc |
| | | gtgtccttgcttccttcatgggtcctctcaacttctctgcg |
| | | ctgagatcccccgcaggcagatcgcggtggagtgttggtgcgatggtgcttgaccccccagcttcccctgctattgggtttc |
| | | tccaacgaggagacatggtcttcgcttctcagagtctgtggggccagggacaggggccantcatggtc |
| | | cccctctcaccctaccctggacgctgtccttgtagctctccgactcgaagaagaagctgcag |
| | | gactttgccagcaccgtggaagctctggaagaggg |
| 512 | 228195 | tgttacctttcagttacatgcctttgatcctaaaattctctacttttgntgccttatcagttctttgcaatctgcctgtggttat |
| | _at | cagcacttaaagcacaattttgaaggggaaaaaaatgataatcaccttagtcccaaagaaataatttntgtcaaactgcctt |
| | | attagtattnaaaacagacacactgaatgaagtagcatgatacgcatatatcctactcagtatcattggccttttatcaaat |
| | | ggggaaactatacttttgtattacatagttttagaaatcgaaagttagagactctttataagtaatgtcaaggaacagtaat |
| | | ttaaaaacaaagttctaacaaatatattgtttgcttaatcacaatgccctcaacttgtatttgaataactaaata |
| | | ggacatgtcttccttggagctgtgggcattagttcagaagc |
| 513 | 228202 | gtttacacctatactgcataatccaacaattttaatttcagttgaagacatgttactaatataactattattaaaagagtagag |
| | _at | gatgtgtaattaaccatatcttctaaaacatggttactaaaagaatatgtaacatcaatattgaccttggtttcttacacaagt |
| | | gttgctaactcaatagtgaaggagacactattaaattttctgaacccatgaga |
| | | gatactagagatggggagtggaaagtgtttggttcagggatatctgaagaacagaagggcagagatttataagtgacgcctcat |
| | | ctacaagctggaaattcctaaaaacaagtagaaagcttataaacaacaggtgatacactcacctcact |
| | | ggttttagtaaattaccaatacagaaagtatccctagtcttaaaaacaagtggaaa |
| | | atttgaactgattagtcatattcctttgattacactgtt |
| 514 | 228232 | cagtcagagtggacaaacctctgtgggaggctctactgcactgagatgcagctcttccgagggggctcctaagccagtgtacaa |
| | _s_at | ctgggtgcgtcttggaacttttcctacaccttctcctggcagcatggttcnagatgaggtgtctggccagctcattctcaccaa |
| | | cctctccctgacctcctcgggcacctaccgctgtgtggccaccaaccagatgggcagtgcatcctgtg |
| 515 | 228241 | gcctcctcagacactctcaagaggatggggagatgacatcacttgggtacaaacttatgaagaaggtctcttttatgctcaaaaa |
| | _at | agtaagaagccattaatggttattcatcacctggaggattgtcaatactctcaagcactaaagaaagtatttgcccaaaatg |
| | | aagaaatacaagaaatggctcagaataagttcatcatgctaaac |
| | | cttatgcatgaaaccactgataagaatttatcacctgatgggcaatatgtgcctagaatcatgtttgtagacccttctttaaca |
| | | gttagagctgacatagctggaagatactctaacagattgtacacatatgagcctcgggatttacccctattgatagaaaacatg |
| | | aagaaagcattaagacttattcagtcagagctataagagatgatagaaaaaagccttcacttcaaagaagtcaa |
| | | atttcatgaagaaaacctctggcacattgacaaat |
| 516 | 228469 | aaggttatagccacattcaacttgaatggtcccnaatgttaaatttaccatgaatactgtgtcttacaatgctgagctctcgact |
| | _at | agactaaccagaataggaacacagtctcaaaggataccataagacaaacagtatttagacatataaaaaactacagagatgataa |
| | | cttgaagaataaataacgtgaaaaactactatattcttgaatgggagtagaatgtaagctctttgagggcagggactttgactgt |
| | | cttgttcattgctatatgataatgcctggcccaacagcaggcattctgtagtgtctgttgaacaaacatctaaaatctggca |
| | | aagtgaacaatggttcctgaggataagtatttaaataacaaaattaactttcactacaaaagctgccaacttacctttatc |
| | | ttctggtgctatcccctgagctttcttaagatca |
| 517 | 228504 | gaatgcatgatctcaagccttaactactataatctttttctgcccctcagaaattgaataacctaaccaagatgcctttagggga |
| | _at | tgccctaagtaaatgtaatttcagatttcagggnttttttttttttcctctctaagtgttccttccctttcttctcctgctctcc |
| | | atcatgttatggagaccagtgaggaaccagtgttaacttggtgacaatgtgacagctggtgctttatctaagctccgttttctat |
| | | ttcttgggaatgctttattgtggaaactgcttcagatacttaaattgaatcataacttgcttctgtaaattgcgtaaagacaaca |
| | | aactgattttagtttgaaaagtttatcttttacttgtaaaccttgtttgccagttacct |
| 518 | 228507 | agccccaggtgtatttctgagtgtcaacagtgttaatttcaagcatgctaataaaatgtnnnnncggtnatnatannnnnnann |
| | _at | naannanncnttnaaantnnnggtttccantctaaaancanccncntttgcttttgttagctgtaatattttttgtcatttag |
| | | ataagacctggtttggctctcaataaaagatgaagacagtagctctgta |
| | | cagggatatatctatattagtcttcatctgatgaatgaagaaattttctcatattatgttcaagaaagtatttacttcctaaaa |
| | | atagaattcccgattctgtctattttggttgaataccagaacaaatctttccgttgcaatcccagtaaaacgaaagaaaa |
| | | ggaatatcttacagactgttcatattagatgtatgtagactgttaatttgcantttccccatatttcctgcctatctta |
| | | cccagataactttctttgaaggtaaaagctgtgcaaaaggcatgagactcaggcctactctttgtttaaat |
| 519 | 228640 | aatcttgtgagttgtgacctaggtgaaggtntaatgataacatctggaggaagcatcatttgttcagctacctagggtttacttt |
| | _at | cagacttttcctacagtttaaagcacttgcgttcagtgtggtacgatctgtttgtaatgttaatcattgactattgttctgctac |
| | | ttggatgttgatagtgaagcagagaacaatttatcatcgtttattatgagtcactatgcacgcaactatgctta |
| | | acatggcgaaatgtattaaacactcgtccaactattt |
| 520 | 228706 | aaagggtggcatttgcgtcacgtggaccagggacagtgctgaaatcagcagtgctcagaaacaatttaacatgttgaaacgacaa |
| | _s_at | tattctaaaatactgatgaatcttgcatcaatataattattgggttttttttctttttcctgctgtataactccttgccatgc |
| | | aaactctcaagaggccaatatattcctggccatgtttgaatgagcctcttaaaataaacttagagccatgcaaatgccagc |
| | | agcttaatggatttcatggaatgaaataccgtgattaactcatagctacatatcattgcataaatgggatttatcttttt |
| | | ctcacttatttttgcggtgaaa |
| 521 | 228707 | agagccatgcaaatgccagcagcttaatggatttcatggaatgaaataccgtgattaactcatagctacatatcattgcataa |
| | _at | atgggatttatcttttttctcacttatttttgcggtgaaagtcgagggcatgcaagagtttctcttccagaagccaagaggaga |
| | | acaaaggtcctaatgctgtactattccaccctttggacgcctcatccaggacgcagaggactctaggtttaacatttt |
| 522 | 228750 | gacccagagtcacctaagagaactcttccaacttaaatgactagggtgtgtaatcagttgcttcttattctgtgtctcagattt |
| | _at | cctagtccatgacatttaatagtgagtatagagtaggatattctatacattagtatttggcccttggaaacaaagttaaat |
| | | accatgtcactcagaagactctcaaagtttgtgtataaatggcgaagtatgca |
| | | ctatgagagattctaatccattcacatcattcttgagttgtgcaaatacacttggctttgatttcacttggtgatctaaattt |
| | | gtttatttaagctgcaagaaagttacattaatttggaatgtgtcatcacttggacccagtatatcagatttttattga |
| | | gtaaaatgttaaaaataataagtgtaactcagattctggatgttcgagtttacaatacattgcctgt |
| | | aataaaagctgattctgaagtgcatttttcttgacttactttggcag |
| 523 | 228766 | atgcacgtacaaatttcttaacctgttatcaatgtctgagctacataattatctttctagttggagtttgttttaggtgtgtac |
| | _at | caactgacatttcagtttttctgtttgaagtccaatgtattagtgactctgtggctgctctcttcacctgccccttgtggcctg |
| | | tctacaattctaaatggattttgaactcaatgtcgtcgcttctggatcctgcatataccaatagcattacctatg |
| | | acttttttttcctgagctattttcactgagctgagctaatgaactaaaactgagttatgtttaatatttgtatcaa |
| | | atacataaaaggaatactgctattccttttgtggctcaaaggtagctgcatt |
| 524 | 228846 | ggaatcttttccaataaccagctaaagatttgcactgaaatacaacttgtatgccttttgcatttttaaagcctgcttcctggat |
| | _at | ttaagcagagtgatagtgttcaaagagccagttcagcctgtaacatatttgaaaaagatatgtctgcacttt |
| | | gaggtcccttttgaatgccattcactagacctctcaagcattttgtttcattgctacatccaagcg |
| | | cctcacaagtccacaatgcgggacagcatcaaaagctcaagactttgnaaaaagcttgtgggcttgcactgggggagggaaggga |
| | | acaaaatttgtgtacttctttgtttaatttagaaataaggcatccaagagatgccattattttctgtgtttcaattgttgtg |
| | | cctttgagttaaactgcatttttgtcttttggttgaaatctgaaatgtactgtcccaatat |
| 525 | 228854 | ctccttatctgttctagttccgaagcagtttcactcgaagttgtgcagtcctggttgcagctttccgcatctgccttcgtttcgt |
| | _at | gtagattgacgcgtttctttgtaatttcagtgtttctgacaagatttaaaaaaaaaaaaaaggaaaaaaaaagaaaaaatg |
| | | aatttactgctgcaggtttttttctctctccatgtgtcactaagtgaagtttgtgccttctatagcaaagagaatat |
| | | tattacatcctactaacagtagatttttttgtagtgaacattttttgtatttttatttataagtctcataagaaaaat |
| | | cagcaatgttcagttgtatacttgaatctgcagttaga |
| 526 | 228885 | aataggtcttgcaggtttccttttgaattttaagtatcataaatattattaagtaaataatacggggtgtcagtaatatctgca |
| | _at | gaatgaatgcagtctttcatgctaatgagttagtctggaaaaataaagtcttattttctatgttttattcatagaaatgga |
| | | gtattaatttttaatattttcaccatatgtgataacaaaggatctttcatgaatgtccaagg |
| | | gtaagtcagtattaattaatgctgtattacaaggcaatgctaccttctttattccccctttgaactacctttgaagtcact |
| | | atgagcacatggatagaaatttaacttttttttgtaaagcaagcttaaaatgtttatgtatacatacccagcaactt |
| | | ttataaatgtgttaaacaattttactgatttttataataaatattttggtaagattttgaataatatgaattcaggca |
| | | gatatactaaactgcttttatttacttgtttagaaaattgtatatatatgtttgtgtatcctaacagctgctatgaa |
| 527 | 228961 | gagaacatcagatctaatagagcatagtgatactatttaatttaaccaaagtctctagtgaatatttcanctttgaatgtaaact |
| | _at | aacaaataaacctgaccaccaaggagattgtttgcccagagtttcaaagcacattgtctacaaatggaaattgaaataatttat |
| | | aaaatattgacgttactatgttttttaaaaagttcctaattttttcactaa |
| | | atggaggaaactattagnattgttaaatatggtagatattaatattcctcttagatgaccagtgattccaattgtcccagttt |
| | | gaaataagtaccctgtgagtatgagataaattagtgacaatcagaacaagtttcagtatcagatgttcaagaggaa |
| | | gttgctattgcattgattttaatatttgtacataaacactgatttttttgagcattattttgtat |
| | | ttgttgtactttaatacctggtgtacagttccagaaa |
| 528 | 229070 | gaaggatgctgaggacctagatgagaagttacctgcaaaaggcaaaagggttacttagtgtcagaaccaaggcaatgacttctct |
| | _at | ctcccagatctcctagctactggtcctgggccanntttttttttttaaataatcccaacntttctttaaaagacaagcattt |
| | | cagtaagctagttattttcatgggttgctcatccatttttttcagtgatctaaaaatgtagggagatggctactactgaa |
| | | gttgtctgtctacttgggataatagcaaattaattgaagacaatgggaaagtaagttataaaaaatactgggaaatctg |
| | | tttctcttctgagcaagcattcagggcaggtataaacatcaaacatagtgacattgtcaaaacctcttccatt |
| 529 | 229254 | ggaaagagaaacgtggctcaagtgctggctcaccttctagctgtgtggccctgggcaggttactgaggctcttccaanctcactt |
| | _at | ttcacatgtaaaatggcatttctaaaagtacctaccctacctcaaaggaattctgaaaaaagtaagtgaatttgtnannna |
| | | agcaaagcactcacaatatgcctgacacacaaactgcttaataaatgttagtaatttttaaaacctcctca |
| | | taagcggcttagaaattattcatgtttggtacatatttattgcaagtcaagcagatccaacttttttgaaagctggtggtcta |
| | | ggagtatagttttaattatatttgtaaatactcaaaagtattagatgccagtctaatttaaatgcagtagactgtag |
| | | cgagtaactgacctgggcaatg |
| 530 | 229510 | gggactcagttcttagagaagtcttttggtcttagagttcatcaagcagagaaggggaaggacatggtccaggaaaataaatca |
| | _at | gcacacaaagagatggggtcttgagggtgcatagtacattctggacagccagttacctgggatgagttgggagggagga |
| | | gaataaggacaaaagaccatctgggcaaaaatcacgaaggggtatgtgtgtcatgtaaaggtgtgccatgatagtta |
| | | ttcatattgctattgtaatattaatatatagtaattaactacacatgacacagctttacatgaccttaagtagttatc |
| | | aacattaccataatagtaatattaataactacaataagagccattattattcacttgagg |
| 531 | 229659 | gttagcaagtgccagcttgtaggctggttgaagtacagaactcagaggaaaaaagaaattaaattttagctttctggagagcag |
| | _s_at | cccctctctggcaccatcaaacacttctttgtttcccttcaacttggaactcttcaaacatcaggggttgtgagggtttg |
| | | gccattcttttatcttgggtccatgtgagtgacagaaatggtgcggcctgggaaagatctccctcctttacattttctcttct |
| | | ccctcctcctccttattctaaaactgtgcctccaacagaggggcaggggctcttgtagagagatccctggcccaggacag |
| | | gagatgccaaatctaatttatctcactgagggc |
| 532 | 229831 | tttcctaccaatttcacattttgcagaaacttgttcacatttccaacaatatcagaattagaaaacagttcagataacaagaaag |
| | _at | attaaaaattagggaaattctgatatcaccataaagcactattttacatttagagattacatttaagataaagtcatca |
| | | tacacaaaaacaataaatatttataactttctctataaggtccgcatatactgtat |
| | | atattgaaacaatctgaatgactagtagatttcatatgaccattgttatttccactttctccaatacttgtattttatgctaca |
| | | tgtaatgaagttggacctttttattatttagtaattcctatatgttcctatacttttcattttcaanatgattgctctat |
| | | tgtttcatgttgtttctagcaatatatctccatgagatatgcactttgtttcatattgaaaagtataaaattt |
| | | atctttcaattcctgtgtgtgtatcctatggttatctgtatgtat |
| 533 | 229839 | tcccaacccggttctggcaaagaatccagttatcccttccatgaagacgcacataactctcttacttggtctttccattaggga |
| | _at | caacataagtcttgttttacatcaaataaaaacaatgttaaaaagtgtgtgaaccttaaaaatggaagtctactagtttacata |
| | | cctacttcagaggacatggaaatgaccatgggcctgcatttcagggaccaaagcaaattaggcctggcctaaaata |
| | | catcagaccttttg |
| 534 | 230087 | gttgaccccaactttggaactcagatcataggatctcccagaggtttcaggtgttcctaagaagcttctaaaggaagttttggaa |
| | _at | gaaagactaataggtgtaaacatataaacatatatattatatatagctatatatatttaaagagatctctacatatatatata |
| | | tataaatacaactactgtactctctgcatctgtacaaagtatgagttaaac |
| | | tgcccaggtagtgtgagatacgtaatgcatgaatgtgaatatcctgaaatctgccttcttagcaacgtgccgcgtgtactggag |
| | | agaggacagttgctcctgggagttctctcaatgggcaaatgtacaagttgatgccgtcttatccaccaacaagccatta |
| | | aggttggtttcctgctgagacttttgtaccggaagcgagaccggcagcactgggggtgtccatatgccacttatgagg |
| 535 | 230264 | ggccaaggcacatttgactcctgagatgaattttttgtggtcataatcaaatacttagttgtttttgatgccccaaaataaagtg |
| | _s_at | agaatggtaatttgccaggaattcttcataacagtatcttacaaaaaacgtgttgctctcttcacagtattatgtgtaaagtca |
| | | ttgtttaaagcacgaatgttccctctggggtacttgttaaagctaaatttattttgcttccctccacttagaagtgctgca |
| | | cactttacagcagcttcctttctttccatngcactgcctagttaacagaagtcttataaaaatttaaaaagacacatttct |
| | | tacaaaaaagagttgaatgaggtaaaatggcattagatggctctatattttttaaagctatgtaattgttcagcgtcactt |
| | | ttctaagtacttatacatatctaaacatgtcttcatggtttatattttcacttatatatgctgggctggattaagctttgt |
| 536 | 230595 | ggggcagagggactatgttgtgagcctngcnaaagaagnttgtgtggggactgtgggcagtgaatgcgttgggaacaatatgga |
| | _at | aaactgggagctgccttggaatctacagggccgnnnnnnnnnnnnnnnnnnnnnnnnnnnnnnnnggctgaagaaaagaag |
| | | aatggtattcatgcagttcccattttacagatgaaaacaagaggactttttctgtgaagtcaagaaagtggttacaaactt |
| | | tcagcctgtccgaattatgtattgcccctcccctttttattaataacattgaagtgtgatgggatggtcaaccactgaagc |
| | | cgtctgttgaaacctgctgggactttttagccattctcttcaacataaagaatgggtgtttttggagggggtgagaggaatg |
| | | gggaaatgttgtcaaagagtacaatgttttagttgagacaggaggaatatattttgttgagatctacagcacagcat |
| 537 | 230788 | gatagcgaatgcactcagggtcagcagtggagtttaaaaattgtctcttttcaacttatttaaatgacagcacctgagaagagga |
| | _at | accgttttacactggatgtttctcatgtagaacaagaaatctttctggaattgatgtttacatgtctgttgt |
| | | tggtcatctctcctgtgtcttaaatactttaatgttggaagagcatagtgtttgggctagtgggttt |
| | | ctgacagcccatgggaatgccctgaaactactgtatctgatgtttgattcgatgaggttccatgttttgttttc |
| 538 | 230830 | gcagctgtggtggtcattataagcatngtccntcctgggaagaagcatccaggcaagcagaaaagaaaagatgcagccaccagaa |
| | _at | aaagaaactccagaagtcctgcatttggatgaggccaaggatcacaacagcctaaacaacctaagagaaactttgctct |
| | | cagaaaagccaaacttggcccaggtggaacttgagttaaaagagagagatgtgctgtcagttttccttccggatgtac |
| | | gcagaaactagagctagtgagggttcagagaagccccatcctaagccagacacatgatgtgggct |
| | | cagctcagtggcctgaaacctctcaggttttagagtctctcccaagaagccgcttttttc |
| 539 | 231120 | tactagttgttttacactctcttttcttattcttagggcttttgtgtatgtctnactnntntttnaannanntncncnnnnannc |
| | _x_at | agaccttaanttttatatttttttaaagtngctaacatagcngnaggcacttaagcatttagtcaatgatattggtagaaata |
| | | gtaaaatacatcctttaaatatatatctaagcatatattttaaaaggagcnaaaataaaaccaaagtgttagtaaattttg |
| | | atttattagatattttagaaaaataatagaattctgaagttttaaaaatgtcagtaattaatttattttcattttcagaa |
| | | atatatgcatgcagttatgttttatttgattgttgacttaggctatgtctgtatacagtaaccaa |
| 540 | 231579 | gagtaggttcggtctgaaaggtgtgggcctttatatttgatccacacacgttggtcttttaaccgtgctgancagaaaacaaaa |
| | _s_at | cnggttaanaananccgggtggnnagntgacagaggnaanccgctnaaataccttcacaataaatagtggcaatatatatata |
| | | gtttaagaaggctccccatttggcatcgtttaatttaaatgttatgaataagcacagctctcttctccaattttcatcctgca |
| | | agcaactcaaaannnttnaaannaannttnnnntgtagttattttcaaatctttgcttgataagtattaagaaatantggact |
| | | tgctgccgtaatttaaagctctgttgantttgtttccgnttgganttttgggggaggggagcactgtgtttatgctggaata |
| | | tgaagtctgagaccttccggtgctgggaacacacaa |
| 541 | 231773 | ggaacctataaatgtagtttcatctgtcgtcaattactgcagaaaattatgtgtatccacaacctagttattttaaaaattatgt |
| | _at | tgactaaatacaaagtttgttttctaaaatgtaaatatttgccacaatgtaaagcaaatcttagctatattttaaatcat |
| | | aaataacatgttcaagatacttaacaatttatttaaaatctaagattgctctaacgtctagtgaaaaaaatatttttaa |
| | | aatttcagccaaataatgcattttatttataaaaatacagacagaaaattagggagaaacctctagttttgccaataga |
| | | aaatgcttcttccattgaataaaagttatttcaaattgaannngtncctttcacangtnnatgattaaatctganttct |
| | | taatnatatatcctatgctgattttcccaaaacatgacccatagtattaaataca |
| 542 | 231925 | tctttttgagtgatagtgccattgaatgagcagtatggaaacagtgttacttgatattttgagctttctcaggtttatctaaat |
| | _at | cagtggtagcttaacaaaacccagactaattgtgtgtaattgtatttttaataaaaggaaagtacatttcctataatagc |
| | | atagtactgtttgcatgtaagagtatgcaaaaccttgtgtgtgtgtgtgtgtgtgtgtgtgtgtgtgtgtcttagtgtgt |
| | | ggtaagcatggcagccaactttgtatctgctatttttagtacgagcagagcttcataattgtggtcactagaactgtact |
| | | taccatggacagttaaaact |
| 543 | 231975 | tctctcagtttaaacatcttaacagagaatttgttactgtttattaaaagaattgcatttggaagcaagaaaacaaccggatt |
| | _s_at | tgcaatcatgtgaagaaacaataatgcctttattatcaagtgttaagcacaattcttataacaaactgtgataa |
| | | attctttttgtttttttttcctttgccccattattttcttatgaacaaaccaaaaattcatggtgagca |
| | | gttgcagtgttggctgatatatcttttatgtacagggaatttgaaaaggacagtggattcatttagaagtgtaactggtgct |
| | | gtgattatagcaatacatttgttagttgtacttcatctttttcatgctagctttttaaatgttt |
| | | agttttcctcttgtcatggtcagctgctgaatttacttgaag |
| 544 | 233565 | agattctggccacggctgggaacgttgtcaccctgaccatcatccccagtgtgatctacgagcacatggtcaaaaagttgcctc |
| | _s_at | cagtcctgctccaccacaccatggaccactccatcccagatgcctgaagccactgcagggcagggcaggcagggggggcttcc |
| | | cgccctcctgcagcaaagggcaaccaccctcggatgatgggttgcagccggcctgctgcttaaggtgggggctgccatgagg |
| | | ggggcgtgtccaggagggtgaccatgggatggcttatacacacaggcctccttggagcctc |
| | | agactccaagctaggctgaggctcaggcagggcccacaggcagccgattctcttgtgctgatttaaatgctggacacggagg |
| | | caggctgtttaaacgctgcttaaagtcgcaactgggcccctttcaagaaattttgctctaccaggaaaaca |
| | | gttacacattttaagagaacagagctacgttctttgtgagagctttttccttgccttgacttg |
| | | ctctttgtcacagactgca |
| 545 | 234764 | gtcaccatctcttgttctggaagcagctccaacatcggaagtaattatgtatactggtaccagcagctcccaggaacggccccc |
| | _at | aaactcctcatctataggaataatcagcggccctcaggggtccctgaccgattctctggctccaagtctggcacc |
| | | tcagcctccctggccatcagtgggctccggtccgaggatgaggctgattatta |
| | | ctgtgcagcatgggatgacagattgagtggttttat |
| 546 | 234987 | gtttactctgagattctgtagtcctaatattgtatcattgtgctgtctgcaaaacaacttgaatctattttcttgcatctttt |
| | _at | gttacatgtaacgcagctgtactttatgttctttgcaactgtttccattatgagaacgctgtgctatttacaaggttacattt |
| | | ttcttggccaggcgaggtggtcatgcctgtaatcccagcactttgggaggccaaggtgggcggatcacttgaggtaaagagtt |
| | | gagaccagcctggctagcatggcgaaacccagtctctactaaaaatancaaaaattagccgggtgaaattagccgggcg |
| 547 | 235146 | ggtgttttaaggttagcacatgcacaaaggaacgagttggtttaaaaagataaatcactgcaaagaatgaaattggcttatt |
| | _at | cacatcaaaactagataagatgctaaaaaaaaaagatatgaaacagaactaacctatagtttcctgaaatcagtaca |
| | | gtttaatttataagaagctagaaagtaatgcaccttgattgttttaggaatgatt |
| | | tatgtgttgcaattttaatttatttaaagcatgtctactgtgtttgtcctaagagaaatatttcaacaaaacgtgctctgtgt |
| | | ttaagatatgtttaggcagtagttagcaactctgaaagtagaaactggaaatgtttattgtgaggcttgttgcagaatttcc |
| | | attttgtgagttactacttagtttcatgtcagcctaaaattgtaaattccctgtagatcttcac |
| | | cccattgtggtgtcatcaatgaatccaaagcaggtgccattattt |
| 548 | 235766 | gcagggtcataatcacacagcagtgccttatatagttgccataagacttcagtgcagtacaacatanttttacngctacatatc |
| | _x_at | agggcatattctatatggtgtatttgtgttagaataacacattaaatgtctttaaacataaaaataagaatgtttgcatgt |
| | | ttcagttttcaagaaccaaatgagtaattagctatagattccactggccttaaacatacaattaagtgtatacatgat |
| | | atagtgcacacacaaaagccacctttaattattgaaataacctgtattctttttggaaatcatttaagtttggtatt |
| | | gaagtactatattttttgtgcatcaatgtatttttctatttacaagcctatgt |
| 549 | 235849 | ggagatcgtgttctgctttttacatgaggagcagaactgggccatacacgtgttcaagaactaggggagctacctggtagcaa |
| | _at | gtgagtgcagacccacctcaccttgggggaatctcaaactcataggcctcagatacacgatcacctgtcatatcaggtgag |
| | | cactggcctgcttggggagagacctgggcccctccaggtgtaggaacagcaacactcctggctgacaactaagccaatat |
| | | ggccctaggtcattcttgcttccaatatgcttgccactccttaaatgtcctaatgatgagaaactctctttctgaccaatt |
| | | gctatgtttacataacacgcatgtactcatgcatcccttgccagagcccatatatgtatgcatatataaacatagcacttt |
| | | ttactacatagctcagcacattgcaaggtttgcatttaagttaaaaaaaaaaagaaaaaaaacctaagggtgaaacgatgc |
| | | cactttgacgc |
| 550 | 236300 | tgactttcatacacacccagtacatctcaaaggatgctaagggacattttctgccagtagagttctccccctttttnggtgaca |
| | _at | gcaatattattatgttcacatctaactccagagcttacttcctgtggtgccaatgtatttgttgcaatttactacattttt |
| | | atatgagcctatttataggtgccattaaactcaggtctttcaaatgaaagagtttctagc |
| | | ccncttagggaaaaagataattgtttagaaaaccataaaatcaatggtaggaaaagttggaactggttacctggatgccatg |
| | | gttctctgttaaataaagtaagagaccaggtgnannnnnaannnnnannanngntattttcagcatgctaataaatgtctt |
| | | tccggttatatatctatctaaaattaacctttaaaatattggtttccttgataaaagcaccactttggcttttgttagctg |
| | | taatattttgtcatttagattaagacctggttaggctctcaataaaagatgaagacagtagctctgtacagcggataatatc |
| 551 | 236313 | gctagttgcattatactgggtcatgaaaaattatcccttgaaatagatatgaaacatgttacttcatttctggtttaaataa |
| | _at | cttgtggaatctttcctaatgacaacntgatattaagggaaactaaagaaaatgttattgtggatcccacagtactatatta |
| | | cactgtttttttttgtttgttttgttagtttttttttatttaaagcaaacctcaaacattattgggt |
| | | atcaattaccacctggttgtattaaaatagtaacttatcaatgccatgtaaaaattaattccattttcgaagccacctggca |
| | | gacaggtttagctgtttcatcagcagcctaatatatactgttaaatttgttaaggatttcactttgaaggatacatgc |
| | | aaaacatatagttactattttcatgagtcctgcttctagctccattgtggaatacagaaaattaaatatac |
| | | ctgttaagttcgtatctaaacctaagacattaccaaggtttgtacaaattctactacctgacatttattccaaga |
| 552 | 238143 | ctcccctgaagaacgtgtccagcaacgcagagtgccctgcttgttatgaatctaatggaacttcctgtngtgggaagccctgga |
| | _at | aatgctatgaagaagaacagtgtgtctttctagttgcagaacttaanaatgacattgagtctaagagtctcgtgctgaaag |
| | | gctgttccaacgtcagtaacgccacctgtcagttcctgtctggtgaaaa |
| | | caagactcttggaggagtcatctttcgaaagtttgagtgtgcaaatgtaaacagcttaacccccacgtntgcaccaaccactt |
| | | cccacaacgtgggctccaaagcttccctctacctcttggcccttgccagcctccttcttcggggactgctgccctgag |
| | | gtcctggggctgcactttgcccagcaccccatttctncttctctgaggtccagagcaccccctgcggtgctgacaccctct |
| | | ttccctgctctgccccgtttaactgcccagtaagtgggagtcacaggtctccaggcaatgccgacagctgccttgt |
| 553 | 238750 | tttgtccatagaggcttcaagatagataggtaagagnccagtagtgttcataagaagccaatagagagcaggagccactttat |
| | _at | caggtggcaggtgtcctgggcctccctgctggctagtcccaagcggtggtgttgccaggatgtcttggaggtgataatgggac |
| | | acacagaggcactgagtctccataggttaaaatgccaccaaaa |
| | | ctggcctttgcctaatatccctcattgactatttagcatttaatttatttattttcctgacatttctgcaagctttgtattta |
| | | tatttccactttatagatgaggaaatttgaggctcttagaggtaaaatgacttgcccaggtcacacaggaagtggcagagaca |
| | | agctttttaaataagaaaaaattaataaaatataatatgagagtaacttaaaatattaataaaccacaattttaaat |
| | | taattaaccgtgataaccaacattaataaaagttaagataccaaaacactggtgtctaattctttcaac |
| 554 | 238751 | gttctgttgtttatccatgaagcctatatatttctagtgcaaaangcccagtgggtctsaaaacattcttggaggggtttgaca |
| | _at | cactgctattagtagtgactcataattccacgattccctgggatacagagaaggcaggggggtagtactgtttgaa |
| | | caaaaatcttcccaagaccctggcaaagagatcttccttaggaatcagtggcatat |
| | | gcgtttatgttctttaacagggaaagtaaaagtgagcgcacttttttgaagctcataaatactcatgtgactgtaaatttaga |
| | | aatgttaattaattgggcctaatgtttgatctggaaaagtacttagcagtcaccaggaatttgccttga |
| | | actgactcgaaccacaaatcagaaccgtatttccacaattta |
| 555 | 239272 | gcactgttaaaatccggctaggccaagatttcccaatgtcttgaagccaatggctaggaaaacagctgagcctgggactggactt |
| | _at | tgtcagtaacctctcttccctagggcacgggaggagtngggtgaataggacctcttggcttgaggaccatgatt |
| | | gtgaatacagacatttaattcagaatgacacctacattgatgaattttcattgtacaagtgtttttccagatc |
| | | aaacacaaaacccatgtaattccttgataaattttcaatgcagggcagataaactgttgctaac |
| 556 | 241994 | gtgatcttggactgtcaattcccctncctgtgatccattttactgcaaacataagggttgcagtaaagggttgtctcacgtctt |
| | _at | ctgctttaaaagcctataaatatatgacctgaaaactccagttacataaaggatctgcagctatctaaggcttg |
| | | gttttcttactgtcatatgatacctgggtctaatgaactctgctgagatcacctcaagtttctg |
| | | cggttggtaaagagaacaagggaagaacaaacatcccttttattgctccaaatggtgatttaatccctacatggtgctgggt |
| | | ggacaatgtgtcactgtcacatgccttnactgtataaatccaaccttctgccagagagaatctgtggttctggcc |
| | | atggagggaggatagtggaaatgatatagttggactggtgcttgatgtcacta |
| 557 | 242317 | atgtgattatacttctctttgacttgtcagcttagctttagnngatacactctggtgcccaactattattgtatcagtgaa |
| | _at | cttnccactttncttttnccttttnctctcaatttttgttgtatcattcctaccttgtgaggacatataatatttacattc |
| | | tgttgtcatcctcacatttcttagttccacagtttaaatgtatttgaaactcaaaacattcccattaatctcttggtcagc |
| | | tgaaattaatgatttaatagtttccttaaaaaagactcatggaacaatttccctaaatttttgccatgtcaaatatgttta |
| | | tctgtagcctttacacagtaaaaacaatttggctagataatacaattctcagttcatat |
| 558 | 242447 | agggcacttctcaacattaattttggaattattcaccatttaaaaattgtcactgctctttttcattactgtagataactg |
| | _at | agagttagctgttctaggaaaaggccatgtttcagttagtgctatgacttctttttttcgattaaggttgaagcagtgt |
| | | tctcttagtagcctgaacacttctcaaagcgacttatcactagcggctttaatattgagcttctatatcgttgtgtc |
| | | cacctcactttttaaggctgttcatacctggcaatattcacttactctgctatttttaaaaactattttaaagtttt |
| | | aagcacaatgttgactcttctgtaatttcctaaaattatattgttttcctacagacagctcaactttccttgatatgt |
| 559 | 242601 | ctcattattcctttacatgcagaatagaggcatttatgcaaattgaactgcagtttttcagcatatacacaatgtcttgt |
| | _at | gcaacagaaaaacatgttggggaaatattcctcagtggagagtcgttctcatgctgacggggagaacgaaagtga |
| | | caggggtttcctcataagttttgtatgaaatatctctacaaacctcaattagttctactctacactttcacta |
| | | tcatcaacactgagactatcctgtctcacctacaaatgtggaaactttacattgttcgatttttcagcagact |
| 560 | 243278 | ataaatgaccagactttttctaagaaaaatgttgctttaatgcatttcatgaatttttactcttatatcattgcttgctagt |
| | _at | aatagcaaatctgcttttctgcatctgctttgcgtagctattgtaaggctttgaactaatgtatgtatttattgcttgaactt |
| | | ctgtgcataccttataaagcataatgtctgacaatttaaatggctcatgtattcttgcttntatcataagctgattatgg |
| | | ggactatgatcttttgtatacagcaaattttaaactgtagcacaaacatctgtttatgtattggtggaatatacctgtttta |
| | | tttatcttttttgaggtaaactaatttttgatacttttcattactgtgtactatgttcatactttgaattctct |
| | | gacgttagaagtcatggttgagaattgtaacagctgttattcgttctgtattcatggctttc |
| 561 | 200832 | aaaagcgaggtggccatgttatgctggtggttgccagggcctccaaccactgtgccactgacttgctgtgtgaccctgggcaagt |
| | _s_at | cacttaactataaggtgcctcagttttccttctgttaaaatggggataataatactgacctacctcaaagggcagttttga |
| | | ggcatgactaatgctttttagaaagcattttgggatccttcagcacaggaattctcaagacctgagtattttttataatag |
| | | gaatgtccaccatgaacttgatacgtccgtgtgtcccagatgctgtcattagtctatatggttctccaagaaactgaatg |
| | | aatccattggagaagcggtggataactagccagacaaaatttgagaatacataaacaacgcattgccacggaaacatacaga |
| | | ggatgccttttctgtgattgggtgggattttttccctttttatgtgggatatagtagttacttgtgacaagaa |
| | | taattttggaataatttctattaatatcaactctgaagctaattgtactaatctgagat |
| 562 | 201147 | gagtcggagatgatgcagcacacacacaattccccagcccagtgatgcttgtgttgaccagatgacctgagtctggagcaagca |
| | _s_at | cccaggccagaataacagagctttcttagttggtgaagacttaaacatctgcctgaggtcaggaggcaatttgcctgccttg |
| | | tacaaaagctcaggtgaaagactgagatgaatgtctttcctctcc |
| | | ctgcctcccaccagacttcctcctggaaaacgctttggtagatttggccaggagctttcttttatgtaaattggataaatac |
| | | acacaccatacactatccacagatatagccaagtagatttgggtagaggatactatttccagaatagtgtttagctca |
| | | cctagggggatatgtttgtatacacatttgcatatacccacatgggg |
| 563 | 201162 | ggaatgtcactggtgcccaggtgtacttgagctgtgaggtcatcggaatcccgacacctgtcctcatctggaacaaggtaaaaa |
| | _at | ggggtcactatggagttcaaaggacagaactcctgcctggtgaccgggacaacctggccattcagacccggggtggcccag |
| | | aaaagcatgaagtaactggctgggtgctggtatctcctctaagtaaggaagatgctggagaatatgagtgccatgcatcca |
| | | attcccaaggacaggcttcagcatcagcaaaaattacagtggttgatgccttacatg |
| 564 | 201163 | aggtgaaggtgccgagctataaacctccagaatattattagttgcatggttaaaagtagtcatggataactacattacctgtt |
| | _s_at | cttgcctaataagtttcttttaatccaatccactaacactttagttatattcactggttttacacagagaa |
| | | atacaaaataaagatcacacatcaagactatctacaa |
| 565 | 201185 | ctcaaagactctcccgtggatgacggatgaggactctgggctgctggaataggacactcaagacttttgactgccattttgttt |
| | _at | gttcagtggagactccctggccaacagaatccttcttgatagtttgcaggcaaaacaaatgtaatgttgcagatccgc |
| | | aggcagaagctctgcccttctgtatcctatgtatgcagtgtgctttttcttgccagc |
| | | ttgggccattcttgcttagacagtcagcatttgtctcctcctttaactgagtcatcatcttagtccaactaatgcagtcgat |
| | | acaatgcgtagatagaagaagccccacgggagccaggatgggactggtcgtgtttgtgcttttctccaagtcagcaccc |
| | | aaaggtcaatgcacagagaccccgggtgggtgagcgctggcttctcaaacggccgaagttgcctctttt |
| 566 | 201261 | tctctctttctgtgtgtgtgtgtgtgtgtgtgtgtgtgtgtgtgtgtgtcttgtgacctcagacctttctcgcttctgagcttg |
| | _x_at | gtggcctgttccctccatctctccgaacctggcttcgcctgtccctttcactccacaccctctggccttctgccttgagctggg |
| | | actgctttctgtctgtccggcctgcacccagcccctgcccacaaaaccccagggacagcggtctccccagcctgccctgctca |
| | | ggccttgcccccaaacctgtactgtcccggaggaggttgggaggtggaggcccagcatcccgcgcagatgacaccatcaaccg |
| | | ccagagtcccagacaccggttttcctagaagcccctcacccccactggcccactggtggctaggtctccccttatccttctgg |
| | | tccagcgcaaggaggggctgcttctgaggtcggtggctgtctttccattaaagaaacacc |
| 567 | 201792 | ccaccgctggctgggaggagtcggagactgagacctacacagaggtggtgacagagtttgggaccgaggtggagcccgagttt |
| | _at | gggaccaaggtggagcccgagtttgagacccagttggagcctgagttcgagacccagctggaacccgagtttgaggaagagga |
| | | ggaggaggagaaagaggaggagatagccactgg |
| | | ccaggcattccccttcacaacagtagagacctacacagtgaactttggggacttctgagatcagcgtcctaccaagaccccag |
| | | cccaactcaagctacagcagcagcacttcccaagcctgctgaccacagtcacatcacccatcagcacatggaaggc |
| | | ccctggtatggacactgaaaggaagggctggtcctgcccctttgagggggtgcaaacatgactgggacctaagag |
| | | ccagaggctgtgtagaggctcctgctccacctgccagtctcgtaagagatggggttgctgcagtgttggagtag |
| | | gggcagagggagggagccaaggtcactcca |
| 568 | 202237 | ctactacatgattggtgagcagaagttctccagcctccccctgggccgggaggcagtagaggctgctgtgaaagaggctgg |
| | _at | ctacacaatcgaatggtttgaggtgatctcgcaaagttattcttccaccatggccaacaacgaaggacttttctc |
| | | cctggtggcgaggaagctgagcag |
| 569 | 202238 | tcgtcactgactactcagaccagaacctgcaggagctggagaagtggctgaagaaagagccagaggcctttgactggtccccag |
| | _s_at | tggtgacctatgtgtgtgatcttgaagggaacagagtcaagggtccagagaaggaggagaagttgagacaggcggtc |
| | | aagcaggtgctgaagtgtgatgtgactcagagccagccactgggggccgtccccttacccccggctgactgcgtgc |
| | | tcagcacactgtgtctggatgccgcctgcccagacctccccacctactgcagggcgctcaggaacctcggc |
| | | agcctactgaagccagggggcttcctggtgatcatggatgcgctcaagagcagctactac |
| 570 | 202310 | tggcctacatggaccagcagactggcaacctcaagaaggccctgctcctccagggctccaacgagatcgagatccgcgccgag |
| | _s_at | ggcaacagccgcttcacctacagcgtcactgtcgatggctgcacgagtcacaccggagcctggggcaagacagtgattga |
| | | atacaaaaccaccaagacctcccgcctgcccatcatcgat |
| | | gtggcccccttggacgttggtgccccagaccaggaattcggcttcgacgttggccctgtctgcttcctgtaaactccctcca |
| | | tcccaacctggctccctcccacccaaccaactttccccccaacccggaaacagacaagcaacccaaactgaacc |
| | | ccctcaaaagccaaaaaatgggagacaatttcacatggactttggaaaatatttttttcc |
| | | tttgcattcatctctcaaacttagtttttatctttgaccaaccgaacatgacca |
| 571 | 202311 | gctccccatttttataccaaaggtgctacatctatgtgatgggtggggtggggagggaatcactggtgctatagaaattgaga |
| | _s_at | tgcccccccaggccagcaaatgttcctttttgttcaaagtctattatattccttgatattttttntttnttttttttttttt |
| | | ttgtggatggggacttgtgaatttttctaaaggtgctatttaacatgggaggagagcgtgtgcggctccagcccagcccgct |
| | | gctcactttccaccctctctccacctgcctctggcttctcaggcctctgctctccgacctctctcctctgaaaccctcctcc |
| | | acagctgcagcccatcctcccggctccctcctagtctgtcctgcgtcctctgtccccgggtttcagagacaac |
| | | ttcccaaagcacaaagcagtttttncccctaggggtgggaggaagcaaaagactctgtacctattttgt |
| 572 | 202404 | actttcccatgagtgtgatccacattgttaggtgctgacctagacagagatgaactgaggtccttgttttgttttgttcata |
| | _s_at | atacaaaggtgctaattaatagtatttcagatacttgaagaatgttgatggtgctagaagaatttgagaagaaat |
| | | actcctgtattgagttgtatcgtgtggtgtattttttaaaaaatttgatttagcattcatattttccatcttattc |
| | | ccaattaaaagtatgcagattatttgcccaaagttgtcctcttcttcagattcagcatttgttctttgccagtctcatttt |
| | | catcttcttccatggttccacagaagctttgtttcttgggca |
| 573 | 202450 | gaacaacgcctgtggcattgccaacctggccagcttccccaagatgtgactccagccagccaaatccatcctgctcttccattt |
| | _s_at | cttccacgatggtgcagtgtaacgatgcactttggaagggagttggtgtgctatttttgaagcagatgtggtgatactgag |
| | | attgtctgttcagtttccccatttgtttgtgcttcaaatgatccttcctactttgcttctctccacccatgacctttttca |
| | | ctgtggccatcaggactttccctgacagctgtgtactcttaggctaagagatgtgactacagcctgcccctgactgtgttgtc |
| | | ccagggctgatgctgtacaggtacaggctggagattttcacataggttagattctcattcacgggactagttagctttaag |
| | | caccctagaggactagggtaatctgacttctcacttcctaagttcccttctatatcctcaaggtagaaatgtctatgtttt |
| | | ctactccaattc |
| 574 | 202859 | gtacccagttaaattttcatttcagataaacaacaaataattttttagtataagtacattattgtttatctgaaagttttaatt |
| | _x_at | gaactaacaatcctagtttgatactcccagtcttgtcattgccagctgtgttggtagtgctgtgttgaattacggaataat |
| | | gagttagaactattaaaacagccaaaactccacagtcaatattagtaatttcttgctggttgaaacttgtttattat |
| | | gtacaaatagattcttataatattatttaaatgactgcatttttaaatacaaggctttatatttttaactttaagat |
| | | gtttttatgtgctctccaaattttttttactgtttctgattgtat |
| 575 | 202878 | atccgaattctccatatattcactaatcaaagacactattttcatactagattcctgagacaaatactcactgaagggcttgtt |
| | _s_at | taaaaataaattgtgattggtctgttcttgtagataatgcccttctattttaggtagaagctctggaatccctttattgtgc |
| | | tgttgctcttatctgcaaggtggcaagcagttcttttcagcagattttgcccactattcctctgagctgaagttc |
| | | tttgcatagatttggcttaagcttgaattagatccctgcaaaggcttgctctgtgatgtcagatgtaattgtaaatgtcagt |
| | | aatcacttcatgaatgctaaatgagaatgtaagtatttttaaatgtgtgtatttcaaatttgtttgactaattctggaattac |
| | | aagatttctatgcaggatttaccttcatcctgtgcatgtttcccaaactgtgaggagggaaggctcagagatcgagcttctcc |
| | | tctgagttctaacaaaatggtgctttgagggtcagcctttaggaaggtgcagctttgttgtcctttg |
| 576 | 202917 | ggcatcatgttgaccgagctggagaaagccttgaactctatcatcgacgtctaccacaagtactccctgataaaggggaatttc |
| | _s_at | catgccgtctacagggatgacctgaagaaattgctagagaccgagtgtcctcagtatatcaggaaaaagggtgcagacgtctgg |
| 577 | 202998 | gccagtcttgaccgggatgaggcccacagacaggttgtcatcagcttgtcccattcaagccaccgagctcaccacagacacagt |
| | _s_at | ggagccgcgctcttctccagtgacacgtggacaaatgcgggctcatcagcccccccagagagggtcaggccgaaccccat |
| | | ttctcctcctcttaggtcattttcagcaaacttgaatatctagacc |
| | | tctcttccaatgaaaccctccagtctattatagtcacatagataatggtgccacgtgttttctgatttggtgagctcagacttg |
| | | gtgcttccctctccacaacccccaccccttgtttttcaagatactattattatattttcacagacttttgaagcacaaat |
| | | ttattggcatttaatattggacatctgggcccttggaagtacaaatctaaggaaaaaccaacccactgtgtaagtgactcat |
| | | cttcctgttgttccaattctgtgggtttttgattcaacggtgctataaccagggtcctgggtgacagggcgctcactgagca |
| | | ccatgtgtcatcacagaca |
| 578 | 203083 | caggaaatagtcactcatcccactccacataaggggtttagtaagagaagtctgtctgtctgatgatggatagggggcaaatct |
| | _at | ttttcccctttctgttaatagtcatcacatttctatgccaaacaggaacgatccataactttagtcttaatgtacaca |
| | | ttgcattttgataaaattaattttgttgtttcctttgaggttgatcgttgtgttgttttgctgca |
| | | ctttttacttttttgcgtgtggagctgtattcccgagacaacgaagcgttgggatacttcattaaatgtagcgactgtcaaca |
| | | gcgtgcaggttttctgtttctgtgttgtggggtcaaccgtacaatggtgtgggaatgacgatgatgtgaatatttagaat |
| | | gtaccatattttttgtaaattatttatgtttttctaaacaaatttatcgtataggttgatgaaacgtcatttgttttgcc |
| | | aaagactgtaaatatttatttatgtgttcacatggtcaaaatttcaccactgaaaccctgcacttagctagaacct |
| 579 | 203325 | cccacctggagctgaatcacatgacctagctgcaccccagcgcctgggcccgccccacgctctgtccacacccangcgccccggg |
| | _s_at | agcggggccatgcctccagccccccagctcgcccgacccatcctgttcgtgaataggtctcaggggttgggggaggga |
| | | ctgccagatttggacactatatttttttctaaattcaacttgaagatgtgtatttcccctgaccttcaaaaaatgtt |
| | | ccaaggtaagcctcgtaaaggtcatcccaccatcaccaaagcctccgtttttaacaacctccaacacgatccattta |
| | | gaggccaaatgtcattctgcaggtgccttcccgatggattaaag |
| 580 | 203570 | ctggaggacagcacctgtgacttcggcaacctcaagcgctatgcatgcacctctcatacccagggcctgagcccaggctgctat |
| | _at | gacacctacaatgcggacatcgactgccagtggatcgacataaccgacgtgcagcctgggaactacatcctcaaggtgc |
| | | acgtgaacccaaagtatattgttttggagtctgacttcaccaacaa |
| | | cgtggtgagatgcaacattcactacacaggtcgctacgtttctgcaacaaactgcaaaattgtccaatcctgatctccggga |
| | | gggacagatggccaatctctccccttccaaagcaggccctgctccccgggcagcctcccgccgaggggcccagcc |
| | | cccaacccacaggcagggaggggcatccctccctgccggcctcagggagcgaacgtggatgaaaaccacaggga |
| | | ttccggatgccagaccccattttatacttcacttttctctacagtgttgttttgttgttgttggtttttatttt |
| | | ttatactttggccataccacagagctagattgccc |
| 581 | 203878 | tgccagcgactgtctcagactgggcagggaggctttggcatgacttaagaggaagggcagtcttgggacccgctatgcag |
| | _s_at | gtcctggcaaacctggctgccctgtctcatccctgtccctcagggtagcaccatggcaggactgggggaactgga |
| | | gtgtccttgctgtatccctgttgtgaggttccttccaggggctggcactgaagcaagggtgctggggccccatg |
| | | gccttcagccctggctgagcaactgggctgtagggcagggccacttcctgaggtcaggtcttggtaggtgcctgcatct |
| | | gtctgccttctggctgacaatcctggaaatctgttctccagaatccaggccaaaaagttcacagtcaaa |
| | | tggggaggggtattcttcatgcaggagaccccaggccctggaggctgcaacatacctcaatcctgtcc |
| | | caggccggatcctcctgaagcccttttcgcagcactgctatcctccaaagccattgtaaatgtgtgt |
| | | acagtgtgtataaaccttcttcttctt |
| 582 | 204006 | gagtacaggtgccagacaaacctctccaccctcagtgacccggtgcagctagaagtccatatcggctggctgttgctccag |
| | _s_at | gcccctcggtgggtgttcaaggaggaagaccctattcacctgaggtgtcacagctggaagaacactgctctgcataag |
| | | gtcacatatttacagaatggcaaagacaggaagtattttcatcataattct |
| | | gacttccacattccaaaagccacactcaaagatagcggctcctacttctgcagggggcttgttgggagtaaaaatgtgtc |
| | | ttcagagactgtgaacatcaccatcactcaaggtttggcagtgtcaaccatctcatcattctctccacctgggtaccaa |
| | | gtctctttctgcttggtgatggtactcctttttgcagtggacacaggactatatttctctgtgaagacaaacat |
| | | ttgaagctcaacaagagactggaaggaccataaacttaaatggagaaaggaccctcaagacaaatgacc |
| 583 | 204051 | aaccagccagtcccaagaagaacattaaaactaggagtgcccagaagagaacaaacccgaaaagagtgtgagctaactagttt |
| | _s_at | ccaaagcggagacttccgacttccttacaggatgaggctgggcattgcctgggacagcctatgtaaggccatgtgc |
| | | cccttgccctaacaactcactgcagtgctcttcatagacacatcttgcagcatttttcttaaggctatgct |
| | | tcagtttttctttgtaagccatcacaagccatagtggtaggtttgcccatggtacagaag |
| 584 | 204320 | gaaaatgtaccttggtgccaccaacccattttgtgccacatgcaagttttgaataaggatgtatggaaaacaacgctgcat |
| | _at | atacaggtaccatttaggaaataccgatgcctttgtgggggcagaatcacagacaaaagctttgaaaatcataaaga |
| | | tataagttggtgtggctaagatggaaacagggctgattcttgattcccaattctcaactctccttttcctatttga |
| | | atttctttggtgctgtagaaaacaaaaaaagaaaaatatatattcataaaaaatatggtgctcattctcatccatc |
| | | caggatgtactaaaacagtgtgtttaataaattgtaattattagtgtacagttctatactgttatctgtgt |
| | | ccatttccaaaacttgcacgtgtccctgaattccgctgactctaatttatgaggatgccgaactct |
| | | gatggcaataatatatgtattatgaaaatgaagttatgatttccgatgaccctaagtcc |
| 585 | 204475 | gaagaactgtctattttctcagtcatttttaacctctagagtcactgatacacagaatataatcttatttatacctcagtttgca |
| | _at | tatttttttactatttagaatgtagccattttgtactgatataatttagttccacaaatggtgggtacaaaaagtcaagttt |
| | | gtggcttatggattcatataggccagagttgcaaagatcttttccagagtatgcaactctgacgttgatcccagagagcagc |
| | | ttcagtgacaaacatatcctttcaagacagaaagagacaggagacatgagtctttgccggaggaaaagcagctcaagaa |
| | | cacatgtgcagtcactggtgtcaccctggataggcaagggataactcttctaacaca |
| 586 | 204620 | tgccgtgctcccaaaacattttaaatgaaagtattggcattcaaaaagacagcagacaaaatgaaagaaaatgagagcagaaagt |
| | _s_at | aagcatttccagcctatctaatttctttagttttctatttgcctccagtgcagtccatttcctaatgtataccagccta |
| | | ctgtactatttaaaatgctcaatttcagcaccgatggccatgtaaataagatg |
| | | atttaatgttgattttaatcctgtatataaaataaaaagtcacaatgagtttgggcatatttaatgatgattatggagccttag |
| | | aggtctttaatcattggttcggctgcttttatgtagtttaggctggaaatggtttcacttgctctttgactgtcagcaagactg |
| | | aagatggcttttcctggacagctagaaaacacaaaatcttgtaggtcattgcacctatctcagccataggtgcagtttgctt |
| | | ctacatgatgctaaaggctgcgaatgggatcctgatggaactaaggactccaatgtcgaactcttctttgctgc |
| 587 | 204811 | ggggaactgagggcaaggggatatagtgatggggctcagatggactgggaggagggggagggtgatgcattaattaatggcttcg |
| | _s_at | ttaattaatgtcatgttgcttgtcgctttctcagtgtgtgtgtgtggtccatgcccactgctggtgccagggtgggtgtccat |
| | | gtgcacccggcctggatgccagctgtgtccttcgggggcgtgcgtgt |
| | | aactgtagtgtagtcaggtgctcaatggagaatataaacatatacagaaaaatatatattttaagtttaaaaaacagaaaaaca |
| | | gacaaaacaatccccatcaggtagctgtctaacccccagctgggtctaatccttctcattacccacccgacctggctgccc |
| | | ctcaccttgggctgggggactggggggccatttccttttctctgccctttttttgttgttctattttgtacagacaag |
| | | ttggaaaaacaacagcgacaaaaaagtcaagaaactttgtaaaatatcgtgtgtgtgattccttgt |
| 588 | 205479 | cccgaccggtgggcatttgtgaggcccatggttgagaaatgaataatttcccaattaggaagtgtaagcagctgaggtctctt |
| | _s_at | gagggagcttagccaatgtgggagcagcggtttggggagcagagacactaacgacttcagggcagggctctgatattcc |
| | | atgaatgtatcaggaaatatatatgtgtgtgtatgtttgcacacttgttgt |
| | | gtgggctgtgagtgtaagtgtgagtaagagctggtgtctgattgttaagtctaaatatttccttaaactgtgtggactgtga |
| | | tgccacacagagtggtctttctggagaggttataggtcactcctggggcctcttgggtcccccacgtgacagtgcct |
| | | gggaatgtacttattctgcagcatgacctgtgaccagcactgtctcagtttcactttcacatagatg |
| | | tccctttcttggccagttatcccttccttttagcctagttcatccaatcctcactgggtgggg |
| 589 | 205765 | caccacctacctatgatgccgtggtacagatggagtaccttgacatggtggtgaatgaaacactcagattattcccagttgcta |
| | _at | ttagacttgagaggacttgcaagaaagatgttgaaatcaatggggtattcattcccaaagggtcaatggtggtgattcca |
| | | acttatgctcttcaccatgacccaaagtactggacagagcctgaggagtt |
| | | ccgccctgaaaggttcagtaagaagaaggacagcatagatccttacatatacacaccctttggaactggacccagaaactgcat |
| | | tggcatgaggtttgctctcatgaacatgaaacttgctctaatcagagtccttcagaacttctccttcaaaccttgtaa |
| | | agaaacacagatccccttgaaattagacacgcaaggacttcttcaaccagaaaaacccattgttctaaaggtggattc |
| | | aagagatggaaccctaagtggagaatgagttattctaaggacttctactttggtcttcaagaaagctgtgccccaga |
| | | acaccagagatttcaacttagtca |
| 590 | 205828 | gaaaatcgatgcagccatttctgataaggaaaagaacaaaacatatttctttgtagaggacaaatactggagatttgatgagaa |
| | _at | gagaaattccatggagccaggctttcccaagcaaatagctgaagactttccagggattgactcaaagattgatgctgtt |
| | | tttgaagaatttgggttcttttatttctttactggatcttcacagttggagtttg |
| | | acccaaatgcaaagaaagtgacacacactttgaagagtaacagctggcttaattgttgaaagagatatgtagaaggcacaata |
| | | tgggcactttaaatgaagctaataattcttcacctaagtctctgtgaattgaaatgttcgttttctcctgcctgtgctgtg |
| | | actcgagtcacactcaagggaacttgagcgtgaatctgtatcttgccggtcatttttatgttattacagggcattcaaatg |
| | | ggctgctgcttagcttgcaccttgtcacatagagtgatctttcccaagagaaggggaagcactcgtgtgcaacagac |
| 591 | 205927 | tccacacacggccaggcctgtttatctacactgctgcccactcctctctccagctccacatgctgtacctggatcattctgaagc |
| | _s_at | aaattccgagcattacatcattttgtccataaatatttctaacatccttaaatatacaatcggaattcaagc |
| | | atctcccattgtcccacaaatgtttggctgtttttgtagttggattgtttgtattaggattcaagca |
| | | aggcccatatattgcatttatttgaaatgtctgtaagtctctttccatctacagagtttagcacatttgaacgttgctggttga |
| | | aatcccgaggtgtcatttgacatggttctctgaacttatctttcctataaaatggtagttagatctggaggtct |
| | | gattttgtggcaaaaatacttcctaggtggtgctgggtacttcttgttgcatcctgtcaggaggcaga |
| | | taatgctggtgcctctctattggtaatgttaagactgctgggtgggtttggagttcttggc |
| 592 | 207173 | gaacatccaagtctttcttcttttttaagttgtcaaagaagcttccacaaaattagaaaggacaacagttctgagctgtaattt |
| | _x_at | cgccttaaactctggacactctatatgtagtgcatttttaaacttgaaatatataatattcagccagcttaaaccc |
| | | atacaatgtatgtacaatacaatgtacaattatgtctcttgagcatcaatcttgttactgctga |
| | | ttcttgtaaatctttttgcttctactttcatcttaaactaatacgtgccagatataactgtcttgtttcagtgagagacgcc |
| | | ctatttctatgtcatttttaatgtatctatttgtacaattttaaagttcttattttagtatacatataaatatca |
| | | gtattctgacatgtaagaaaatgttacggcatcacacttatatttta |
| 593 | 207191 | aatctcttctagagcacctgctatccccaacttctagacctgctccaaactagtgactaggatagaatttgatcccctaa |
| | _s_at | ctcactgtctgcggtgctcattgctgctaacagcattgcctgtgctctcctctcaggggcagcatgctaacggggcgacg |
| | | tcctaatccaactgggagaagcctcagtggtggaattccaggcactgtgactgtc |
| | | aagctggcaagggccaggattgggggaatggagctggggcttagctgggaggtggtctgaagcagacagggaatgggaga |
| | | ggaggatgggaagtagacagtggctggtatggctctgaggctccctggggcctgctcaagctcct |
| | | cctgctccttgctgttttctgatgatttgggggcttgggagtccctttgtcctcatctgagactgaaatgtg |
| | | gggatccaggatggcttccttcctcttacccttcctccctcagcctgcaacctctatcctggaacctgtcctccctt |
| | | tctccccaactatgcatctgttgtctgctcct |
| 594 | 208063 | aagaagctaagcctgatctcctgtaaaaacatcatttccctgatggacaccagcggcaatgggaagctggagtttgatgaattca |
| | _s_at | aagtgttctgggacaagctgaagcagtggattaaccttttccttcggtttgatgctgacaagtccggcaccatgtcta |
| | | cctatgaactacggactgcactgaaagctgcaggctttcagctgagcag |
| | | ccacctcctgcagctgattgtgctcaggtatgcggatgaggagctccagctggacttcgatgacttcctcaactgcctggtccg |
| | | gctggagaatgcgagccgggtgttccaggctctcagtacaaagaacaaggagttcattcatctcaata |
| | | taaatgagttcatccatttgacaatgaacatctgaggctgccttgtagagatgcagcctgcc |
| | | cagctgaatcttggcttctggaccttgaccttcagaacttctcttggtgtggaaccattacgcccagggttcactc |
| 595 | 208782 | actcccaagagcaaatccacattcctcttgagttctgcagcttctgtgtaaatagggcagctgtcgtctatgccgtagaatca |
| | _at | catgatctgaggaccattcatggaagctgctaaatagcctagtctggggagtcttccataaagttttgcatggag |
| | | caaacaaacaggattaaactaggtttggttccttcagccctctaaaagcatagggctt |
| | | agcctgcaggcttccttgggctttctctgtgtgtgtagttttgtaaacactatagcatctgttaagatccagtgtccatggaa |
| | | acattcccacatgccgtgactctggactatatcagtttttggaaagcagggttcctctgcctgctaacaagcccacgtggacc |
| | | agtctgaatgtctttcctttacacctatgtttttaagtagtcaaacttcaagaaacaatctaaacaagtttctgttgcatat |
| | | gtgtttgtgaacttgtatttgtatttagtaggcttctatattgcatttaacttgtttttgtaactcctgattcttcct |
| 596 | 208850 | gaagcctcaagttccagtgcagagatcctacttctctgagtcagctgaccccctccccgcaatccctcaaaccttgaggagaa |
| | _s_at | gtggggaccccacccctcatcaggagttccagtgctgcatgcgattatctacccacgtccacgcggccatggtttat |
| | | cctcaccctctccgcacacctctggctgtctttttgtactttttgttccagagctgcttctgtc |
| | | ttaggttttatccttccttttctttgagagttcgtgaagagggaagccaggattggggacctgatggagagtgagagcatg |
| | | tgaggggtagtgggatggtggggtaccagccactggaggggtcatccttgcccatcgggaccagaaacctggga |
| | | gagacttggatgaggagtggttgggctgtgcctgggcctagcacggacatggtctgtcctgacagcactcctc |
| | | ggcaggcatggctggtgcctg |
| 597 | 208851 | aaagcagcgctagtggagggttggagaaggaggtaaggatgagggttcatcatccctccctgcctaaggaagctaaaagcatgg |
| | _s_at | ccctgctgcccctccctgcctccacccacagtggagagggctacaaaggaggacaagaccctctcaggctgtcccaagct |
| | | cccaagagcttccagagctctgacccacagcctccaagtc |
| | | aggtggggtggagtcccagagctgcacagggtttggcccaagtttctaagggaggcacttcctcccctcgcccatcagtgcca |
| | | gcccctgctggctggtgcctgagcccctcagacagccccctgccccgcaggcctgccttctcagggacttctgcggggcc |
| | | tgaggcaagccatggagtgagacccaggagccggacacttctcaggaaatggcttttcccaacccccagcccccacccgg |
| | | tggttcttcctgttctgtgactgtgtatag |
| 598 | 208937 | ttcagccagtcgccaagaatcatgaaagtcgccagtggcagcaccgccaccgccgccgcgggccccacgtgcgcgctgaaggcc |
| | _s_at | ggcaagacagcgagcggtgcgggcgaggtggtgcgctgtctgtctgagcagagcgtggccatctcgcgctgccggggcg |
| | | ccggggcgcgcctgcctgccctgctggacgagcagcaggtaaacgtgctgctctacgacatgaacggctgttactcacg |
| | | cctcaaggagctggtgcccaccctgccccagaaccgcaaggtgagcaaggtggagattctccagcacgtcatcgacta |
| | | catcagggaccttcagttggagctgaactcggaatccgaagttggaacccccgggggccgagggctgccggtccgggct |
| | | ccgctcagcaccctcaacggcgagatcagcgcc |
| 599 | 209218 | gattccctgcatcaactaagaaaagcctgttttctttatttcaaacttggtggcgaatgtgttgcgggtcctgttgggctgcttt |
| | _at | ctgtattgtctcctaaccctctagttttaattggacacttctttgctgttgcaatctatgccgtgtatttttgctttaagtca |
| | | gaaccttggattacaaaacctcgagcccttctcagtagtggtgctgtattgtacaaagcgtgttctgtaatatttcctcta |
| | | atttactcagaaatgaagtatatggttcattaagcttaaaggggaaccatttgtgaatgaatatttggaacttaccaagtc |
| | | ctaagagacttttggaagaggatatatatagcatagtaccataccacttata |
| 600 | 209395 | gctgtggggatagtgaggcatcgcaatgtaagactcgggattagtacacacttgttgatgattaatggaaatgtttacagatcc |
| | _at | ccaagcctggcaagggaatttcttcaactccctgccccctagccctccttatcaaaggacaccattttggcaagctct |
| | | atcaccaaggagccaaacatcctacaagacacagtgaccatactaattataccccctgcaaagcc |
| | | agcttgaaaccttcacttaggaacgtaatcgtgtcccctatc |
| 601 | 209396 | tcaccaatgccatcaaggatgcactcgctgcaacgtagccctctgttctgcacacagcacgggggccaaggatgccccgtccccc |
| | _s_at | tctggctccagctggccgggagcctgatcacctgccctgctgagtcccaggctgagcctcagtctccctcccttggggcctatg |
| | | cagaggtccacaacacacagatttgagctcagccctggtgggcagagaggtagggatggggctgtggggatagtgaggcatcgc |
| | | aatgtaagactcgggattagtacacacttgttgatgattaatggaaatgtttacagatccccaagcctggcaagggaatttctt |
| | | caactccctgccccctagccctccttatcaaaggacaccattttggcaagctctatcaccaaggagccaaacatcctacaag |
| | | acacagtgaccatactaattataccccctgcaaagccagcttgaaaccttcact |
| 602 | 209596 | tccaacctccttcaaattcagtcaccactgttatattaccttctccaggaaccctccagtggggaaggctgcgatattagatt |
| | _at | tccttgtatgcaaagtttttgttgaaagctgtgctcagaggaggtgagaggagaggaaggagaaaactgcatcataactttac |
| | | agaattgaatctagagtcttccccgaaaagcccagaaacttctctgcagtatctggcttgtccatctggtctaaggtggctgc |
| | | ttcttccccagccatgagtcagtttgtgcccatgaataatacacgacctgttatttccatgactgctttactgtattttt |
| 603 | 209875 | gaatggtgcatacaaggccatccccgttgcccaggacctgaacgcgccttctgattgggacagccgtgggaaggacagttatgaa |
| | _s_at | acgagtcagctggatgaccagagtgctgaaacccacagccacaagcagtccagattatataagcggaaagctaatgatgagagca |
| | | atgagcattccgatgtgattgatagtcaggaactttccaaagtcagccgtgaattccacagccatgaatttcacagccatgaaga |
| | | tatgctggttgtagaccccaaaagtaaggaagaagataaacacctgaaatttcgta |
| | | tttctcatgaattagatagtgcatcttctgaggtcaattaaaaggagaaaaaatacaatttctcactttgcatttagtcaaaag |
| | | aaaaaatgctttatagcaaaatgaaagagaacatgaaatgcttctttctcagtttattggagaatgtg |
| | | tatctatttgagtctggaaataactgatgtgtttgataattagtttagtttgtggcttcatggaa |
| 604 | 209955 | acagattccaaggtgacaaactcctctatgcagtgtatcgaaagctgggtgtttatgaagttgaagaccagattacagctgtcag |
| | _s_at | aaaattcatagaaatgggtttcattgatgaaaaaagaatagccatatggggctggtcctatggaggatacgtttcatcact |
| | | ggcccttgcatctggaactggtcttttcaaatgtggtatagcagtggct |
| | | ccagtctccagctgggaatattacgcgtctgtctacacagagagattcatgggtctcccaacaaaggatgataatcttgagca |
| | | ctataagaattcaactgtgatggcaagagcagaatatttcagaaatgtagactatcttctcatccacggaacagcagatg |
| | | ataatgtgcactttcagaactcagcacagattgctaaagctctggttaatgcacaagtggatttccaggcaatgtggtactc |
| | | tgaccagaaccacggcttatccggcctgtccacgaaccacttatacacccacatgacccacttcctaaagcagtg |
| 605 | 210095 | tcttttgtcctccttagcacaatgtaaaaaagaatagtaatatcagaacaggaaggaggaatggcttgctggggagcccatccag |
| | _s_at | gacactgggagcacatagagattcacccatgtttgttgaacttagagtcattctcatgcttttctttataattcacacatatat |
| | | gcagagaagatatgttcttgttaacattgtatacaacatagccccaaatatagtaagatctatactagataatcctagatga |
| | | aatgttagagatgctatatgatacaactgtggccatgactgaggaaaggagctcacgcccagagactgggctgctctcccg |
| | | gaggccaaacccaagaaggtctggcaaagtcaggctcagggagactctgccctgctgcagacctcggtgtggacacacgct |
| | | gcatagagctctccttgaaaacagaggggtctcaagacattctgcctacctattagc |
| 606 | 210511 | aaaggagcagtcgcacagacctttcctcatgctgcaggcccggcagtctgaagaccaccctcatcgccggcgtcggcggggcttg |
| | _s_at | gagtgtgatggcaaggtcaacatctgctgtaagaaacagttctttgtcagtttcaaggacatcggctggaatgactggatca |
| | | ttgctccctctggctatcatgccaactactgcgagggtgagtgcccgagccatatagcaggcacgtccgggtcctcactgtcc |
| | | ttccactcaacagtcatcaaccactaccgcatgcggggccatagcccctttgccaacctcaaatcgtgctgtgtgcccaccaa |
| | | gctgagacccatgtccatgttgtactatgatgatggtcaaaacatcatcaaaaaggacattcagaacatgatcgtggaggagt |
| | | gtgggtgctcatagagttgcccagc |
| 607 | 211571 | acaagcatcctgtctcacgaagaacaaatgtttgttaatcgtgtgggccatgattatcagtggataggcctcaatgacaagatgt |
| | _s_at | ttgagcatgacttccgttggactgatggcagcacactgcaatacgagaattggagacccaaccagccagacagcttctttc |
| | | tgctggagaagactgtgttgtaatcatttggcatgagaatggccag |
| | | tggaatgatgttccctgcaattaccatctcacctatacgtgcaagaaaggaacagttgcttgcggccagccccctgttgtagaa |
| | | aatgccaagacctttggaaagatgaaacctcgttatgaaatcaactccctgattagataccactgcaaagatggtttcattc |
| | | aacgtcaccttccaactatccggtgcttaggaaatggaagatgggctatacctaaaattacctgcatgaacccatctgcatac |
| | | caaaggacttattctatgaaatactttaaaaattcctcatcagcaaaggacaattcaataaatacatccaaa |
| | | catgatcatcgttggagccggaggtgg |
| 608 | 211966 | tggtgatgtctgctactatgccagccggaacgacaagtcctactggctctctaccactgcgccgctgcccatgatgcccgtggcc |
| | _at | gaggacgagatcaagccctacatcagccgctgttctgtgtgtgaggccccggccatcgccatcgcggtccacagtca |
| | | ggatgtctccatcccacactgcccagctgggtggcggagtttgtggatc |
| | | ggatattccttcctcatgcacacggcggcgggagacgaaggcggtggccaatcactggtgtcaccgggcagctgtctagaggac |
| | | ttccgcgccacaccattcatcgaatgcaatggaggccgcggcacctgccactactacgccaacaagtacagcttctggctg |
| | | accaccattcccgagcagagcttccagggctcgccctccgccgacacgctcaaggccggcctcatccgcacacac |
| | | atcagccgctgccaggtgtgcatgaagaacctgtgagccggcgcgtgccaggaagggccattttggtgc |
| | | ttattcttaacttattacctcaggtgccaacccaaaa |
| 609 | 211980 | gaaagactgtgctgtcctttaacataggtttttaaagactaggatattgaatgtgaaacatccgttttcattgttcacttctaa |
| | _at | accaaaaattatgtgttgccaaaaccaaacccaggttcatgaatatggtgtctattatagtgaaacatgtactttgagct |
| | | tattgtttttattctgtattaaatattttcagggttttaaacactaatcacaaactgaatga |
| | | cttgacttcaaaagcaacaaccttaaaggccgtcatttcattagtattcctcattctgcatcctggcttgaaaaacagctctg |
| | | ttgaatcacagtatcagtattttcacacgtaagcacattcgggccatttccgtggtttctcatgagc |
| | | tgtgttcacagacctcagcagggcatcgcatggaccgcaggagggcagattcggaccact |
| 610 | 211981 | tcggctactcttttgtgatgcacaccagcgctggtgcagaaggctctggccaagccctggcgtcccccggctcctgcctggagga |
| | _at | gtttagaagtgcgccattcatcgagtgtcacggccgtgggacctgcaattactacgcaaacgcttacagcttttggctcgccac |
| | | catagagaggagcgagatgttcaagaagcctacgccgtccaccttgaaggcaggggagctgcgcacgcacgtcagccgctgc |
| | | caagtctgtatgagaagaacataatgaagcctgactcagctaatgtcacaacatggtgctacttcttcttctttttgttaac |
| | | agcaacgaaccctagaaatatatcctgtgtacctcactgtccaatatgaaaaccgtaaagtgccttataggaatttgcgtaa |
| | | ctaacacaccctgc |
| 611 | 212344 | ggaaaacacctcatttgaccttgccagctgaccttcaaaccctgcatttgaaccgaccaacattaagtccagagagtaaacttga |
| | _at | atggaataacgacattccagaagttaatcatttgaattctgaacactggagaaaaaccgaaaaatggacggggcatgaagagac |
| | | taatcatctggnaaaccgatttcagtggcgatggcatgacagagctagagctcgggcccagccccaggctgcagcccattcgc |
| | | aggcacccgaaagaacttccccagtatggtggtcctggaaaggacatttttgaagatca |
| | | actatatcttcctgtgcattccgatggaatttcagttcatcagatgttcaccatggccaccgcagaacaccgaagtaattcca |
| | | gcatagcggggaagatgttgaccaaggtggagaagaatcacgaaaaggagaagtcacagcacctagaaggcag |
| | | cgcctcctcttcactctcctctgattagatgaaactgttaccttacccta |
| 612 | 212353 | aatatccttgttgtgtattaggtttttaaataccagctaaaggattacctcactgagtcatcagtaccctcctattcagctcc |
| | _at | ccaagatgatgtgtttttgcttaccctaagagaggttttcttcttatttttagataattcaagtgcttagataaattatgt |
| | | tttctttaagtgtttatggtaaactcttttaaagaaaatttaatatgttatagctgaatctttttggtaactttaaat |
| | | ctttatcatagactctgtacatatgttcaaattagctgcttgcctgatgtgtgtatcatcggtgggatgacagaacaa |
| | | acatatttatgatcatgaataatgtgctttgtaaaaagatttcaagttattaggaagcatactctgttttttaatca |
| 613 | 212354 | gtgtgcacacggagactcatcgttataatttactatctgccaagagtagaaagaaaggctggggatatttgggttggcttg |
| | _at | gttttgattttttgcttgtttgtttgttttgtactaaaacagtattatcttttgaatatcgtagggacataa |
| | | gtatatacatgttatccaatcaagatggctagaatggtgcctttctgagtgtctaaaacttgacacccctgg |
| | | taaatattcaacacacttccactgcctgcgtaatgaagttttgattcatttttaaccactggaatttttcaatgccgtca |
| | | ttttcagttagatgattttgcactttgagattaaaatgccatgtctatttgattagtcttatttttttatttttac |
| | | aggcttatcagtctcactgttggctgtcattgtgacaaagtcaaataaacccccaaggacgacacacagtat |
| | | ggatcacatattgtttgacattaagcttttgccagaaaatgttgcatgtgttttacctcgactt |
| 614 | 212488 | gaaaggcgatctcttcactgtgaaaagttgcccgggtgcagcgccttttccttctaccatgggaaatgcaggctgggcccttggg |
| | _at | gtgagcctgcggggctctggtgctgtccccgacccccaccaccaccagaatgcagttccagcttaggaagccacaaacaagccac |
| | | ccaggaggaacaaaacaccgccagcgtggattttcc |
| | | aaatttccctggaaagtaagtctcgctcttgccaaagaaaagtctggcttggagagtctctggagcccaggatgccagcatgtg |
| | | ccaatgactgtcaccttcatctcttcaaaagaaaagccatagccgaggactgtcccgcgacccccgtggactgcgtctaggt |
| | | catgtgattctgttttcatttctcatcccatccaatttgtccttttctcctgtcattttcttcctctgtggtcccttcaa |
| | | agttgttataatttgtactgaacttcaaaatgtgtcccgttctccccagaccactctagccacagtatatt |
| 615 | 212489 | attgaaaagcaggtaccagtgccccttttcagacagtttttgattcgctctagacnttttttnttttttaatagggagggaaaa |
| | _at | aatttgataancttntttctacatgcacttaagactaaaacacaggtttggattaattttatttgcttcctttttccgcttt |
| | | tcttcccgcagagcctgatgggagaatgtccagggcagggaaaccacattttttgtag |
| | | gtgataactcaatgaaaattggtgcttattttttacacttctctcttgtggctctcttgtggtgctatctgttttaaggtctc |
| | | cttgaaggcgcactggggtccctggccatgcctcgttctccctgctttctttatcctgttattgcctccacagtctgag |
| | | ccaaggactctaagatcaatgcacgtcactttccntccactgggcaggatagccaagcacactccctcctgcgctctc |
| | | ccgccccggtgcgtccactcccgagggctgttatgaggactgggttgtgcctacttgatttgaaaacacacac |
| 616 | 213106 | acttctcagcaaataaatctcccttaagtaggaaanctagatttcatattngcttnattgaattaacagcaactttccacag |
| | _at | gtaaatctgttcttgcaaagatgtgagcagaatagttaaaaataatatttttatgtttcatggttctaaatggaagccat |
| | | aaatgcagtaaatactatctgttgtttaactactttaatcgtcattnacattttcaagttt |
| | | attaggttaagaaaaacagggcagccttggaaggcagctactacagaaaactgcagttttgcgttaaagataaagtagtatt |
| | | ttcagctccctgaaaaaccattcctgctgaaactgctgtagaaattgtgaagctgcatgagtggagagtattgaatct |
| | | gtggttatagtagttttctcaggtttgtttatcttgatgtttgatgcactgtgttttatagttattaaaattgagta |
| | | atattatttctatgcagtgttatgtgtcattggccttttgtgaatgtgcatgttttaaactgcaaattttaaacattttgt |
| | | cctctaattgttat |
| 617 | 213125 | gaatgagtattcctatacgacccagatagactacaaccccaaggaccgcctgctctatgcctgggacaatggccaccaggtcac |
| | _at | ttaccatgtcatctttgcctactgacacccttgtccccacaagcagaagcacagaggggtcactagcaccttgtgtgtatg |
| | | tgtgtgcgcgcacgtgtgtgtaggtgggtatgtgttgtttaaaaatatatattattttgtataatattgcaaatgtaaaatg |
| | | acaatttgggtctatttttttatatggattgtagatcaatccatacgtgtatgtgctggtctcatcctccccagtttatat |
| | | ttttgtgcaaatgaacttctccttttgaccagtaaccaccttccttcaagccttcagcccctccagctcca |
| | | agtctcagatctcgaccattgaaaaggtttcttcatctgggtcttgcaggaggcaggcaacac |
| 618 | 213524 | gtgctcggcctgatggagactgtgtgcagccccttcacggccgccagacgtctgcgggaccaggaggcagccgtggcggagct |
| | _s_at | gcaggccgccctggagcgacaggctctccagaagcaagccctgcaggagaaaggcaagcagcaggacacggtcctcggcggcc |
| | | gggccctgtccaaccggcagcacgcctcctagga |
| | | actgtgggagaccagcggagtgggagggagacgcagtagacagagacagactgagaaggaagggagagacagagggggcgcgc |
| | | gcacaggagcctgactccgctgggagagtgcaggagcacgtgctgttttttatttggacttaacttcagagaaaccgctgac |
| | | atctagaactgacctaccacaagcatccaccaaaggagtttgggattgagtttntgctgctgtgcagcactgcat |
| | | tgtcatgacatttccaacactgtgtgaattatctaaatgcgtctaccattttgcactagggag |
| 619 | 213869 | cacagcctccaagtcaggtggggtggagtcccagagctgcacagggtttggcccaagtttctaagggaggcacttcctccc |
| | _x_at | ntcgcccatcagtgccagcccctgctggctggtgcctgagcccctcagacagccccctgccccgcaggcctgccttctc |
| | | agggacttctgcggggcctgaggcaagccatggagtgagacccagg |
| | | agccggacacttctcaggaaatggcttttcccaacccccagcccnccacccggtggttcttcctgttctgtgactgtgtatagt |
| | | gccaccacagcttatggcatctcattgaggacaaagaaaactgcacaataaaaccaagcctctggaatctgtcctcgtgtcca |
| | | cctggccttcgctcctccancagtgcctgcctgnccccncttcgctggggtctccacgggtgaggctggggaacgccacc |
| | | tcttcctcttccctgacttctccccaaccacttagtagcaacgctaccccaggggctaatgactgcacactg |
| | | ggcttcttttcagaatgaccctaacgagacacatttgcccaaa |
| 620 | 213905 | cacaaaaccccagggacagcggtctccccagcctgattgctcangccttgcccccaaacctgtactgtcccggaggaggt |
| | x_at | tgggaggtggaggcccagcatcccgcgcagatgacaccggttttcctagaagcccctcacccccactggcccactggtg |
| | _ | gctaggtctccccttatccttctggtccagcgcaaggaggggctgcttctgaggtcggtggctgtctttc |
| | | cattaaagaaacaccgtg |
| 621 | 214234 | gaatgaagaaaagtcgcctcaacgacaaacaaaagcaccgactagatttccttcagctgatgattgactcccagaatt |
| | _s_at | cgaaagaaactgagtcccacaaagctctgtctgatctggagctcgcagcccagtcaataatcttcatttt |
| | | tgctggctatgaaaccaccagcagtgttctttccttcactttatatgaactggccactcaccctga |
| | | tgtccagcagaaactgcaaaaggagattgatgcagttttgcccaata |
| 622 | 214235 | ggtgaggggatgacccctggagatgaagggaagaggtgaagccttagcaaaaatgcctcctcaccactccccaggagaattttt |
| | _at | ataaaaagcataatcactgattccttcactgacataatgtaggaagcctctgaggagaaaaacaaagggagaaacatagagaa |
| | | cggttgctactggcagaagcataagatctttgtacaatattgctggccctggttcacctgtttactgttatcacaata |
| 623 | 214247 | acttaggtaattgtagggcgaggattataaatgaaatttgcaaaatcacttagcagcaactgaagacaattatcaaccacgtg |
| | _s_at | gagaaaatcaaaccgagcagggctgtgtgaaacatggttgtaatatgcgactgcgaacactgaactctacgccactccaca |
| | | aatgatgttttcaggtgtcatggactgttgccaccatgtattcatccagagttcttaaagtttaaagttgcacatgattg |
| | | tataagcatgctttctttgagttttaaat |
| 624 | 215646 | tctcacctatacgtgcaagaaaggaacagttgcttgcggccancccctgttgtagaaaatgccaagacctttggaaagat |
| | _s_at | gaaacctcgttatgaaatcaactccctgattagataccacntgcaaagatggtttcattcaacgtcaccttcca |
| | | actatccggtgcttaggaaatggaagatgggctatacctaaaattacctgcatgaacc |
| 625 | 217430 | agggcctaagggtgacagaggtgatgctggtcccaaaggtgctgatggctctcctggcaaagatggcgtccgtggtctga |
| | __x_at | ccggccccattggtcctcctggccctgctggtgcccctggtgacaagggggaccccattcccgaggagctttatgag |
| 626 | 217763 | aacattgtaatggccatcgctggaaacaagtgcgacctctcagatattagggaggttcccctgaaggatgctaaggaata |
| | _s_at | cgctgaatccataggtgccatcgtggttgagacaagtgcaaaaaatgctattaatatcgaagagctctttcaagga |
| | | atcagccgccagatcccacccttggacccccatgaaaatggaaacaatggaa |
| | | caatcaaagttgagaagccaaccatgcaagccagccgccggtgctgttgacccaagggcgtggtccacggtacttgaa |
| | | gaagccagagcccacatcctgtgcactgctgaaggaccctacgctcggtggcctggcacctcactttgaga |
| | | agagtgagcacactggctttgcatcctggaaggcctgcagggggcggggcaggaaatgtacctgaaaagga |
| | | ttttagaaaaccctgggaaacccaccacaccaccacaaaatggcctttagtgt |
| 627 | 218211 | gccacaccttcgcgaaacctgtggtggcccaccagtcctaacgggacaggacagagagacagagcagccctgcactgt |
| | _s_at | tttccctccaccacagccatcctgtccctcattggctctgtgctttccactatacacagtcaccgtcccaatgagaaa |
| | | caagaaggagcaccctccacatggactcccacctgcaagtggacagcgaca |
| | | ttcagtcctgcactgctcacctgggtttactgatgactcctggctgccccaccatcctctctgatctgtgagaaacag |
| | | ctaagctgctgtgacttccctttaggacaatgttgtgtaaatctttgaaggacacaccgaagacctttatact |
| | | gtgatcttttacccctttcactcttggctttcttatgttgc |
| 628 | 218638 | ctgccccgagctcgaagaagaggctgagtgcgtccctgataactgcgtctaagaccagagccccgcagcccctggggcccccg |
| | _s_at | gagccatggggtgtcgggggctcctgtgcaggctcatgctgcaggcggccgaggcacagggggtttcgcgctgctcctgac |
| | | cgcggtgaggccgcgccgaccatctctgcactgaagggcc |
| | | ctctggtggccggcacgggcattgggaaacagcctcctcctttcccaaccttgcttcttaggggcccccgtgtcccgtctgc |
| | | tctcagcctcctcctcctgcaggataaagtcatccccaaggctccagctactctaaattatggtctccttataagttattg |
| | | ctgctccaggagattgtccttcatcgtccaggggcctggctcccacgtggttgcagatacctcagacctggtgctctag |
| | | gctgtgctgagcccactctcccgagggcgcatccaagcgggggccacttgagaagtgaataaatggggcggtttcggaa |
| | | gcgtcagtgtttccatgttatgg |
| 629 | 219955 | gaagttgcaacattcgtttgataggaattccagaaaaggagagttatgagaatagggcagaggacataattaaagaaata |
| | _at | attgatgaaaactttgcagaactaaagaaaggttcaagtcttgagattgtcagtgcttgtcgagtacctagtaaaattg |
| | | atgaaaagagactgactcctagacacatcttggtgaaattttggaattctagtgataaagagaaaataataagggct |
| | | tctagagagagaagagaaattacctaccaaggaacaagaatcaggttgacagcagacttatcactggacacact |
| | | ggatgctagaagtaaatggagcaatgtcttcaaagttctgctggaaaaaggctttaatcctagaatcctatat |
| | | ccagccaaaatggcatttgattttaggggcaaaacaaaggtatttcttagtattgaagaatttagagatt |
| | | atgttttgcatatgcccaccttgagagaattactggggaataatataccttagcacgccagggtgactaca |
| 630 | 221011 | gagtggttcatccatactctcattccctcgcctccccttgtggacgggggtcttgccttttcaattcctgtgttttggtgtct |
| | _s_at | tcccttatctgctaccctgaatcacctgtcctggtcttgctgtgtgatgggaacatgcttgtaaactgcgtaacaaatcta |
| | | ctttgtgtatgtgtctgtttatgggggtggtttattatttttgctggtccctagaccactttgtatga |
| | | ccgtttgcagtctgagcaggccaggggctgacagctaatgtcaggaccctcagcggtggagcctgctggggggacccagct |
| | | gctcttggacaagtggctgagctcctatctggcctcctcttttttttttttcaagtaatttgtgtgtatttc |
| | | taactgattgtattgaaaaaattcctagtatttcagtaaaaatgcctgttgtgagatgaacctcctgt |
| | | aacttctatctgttcttttttgaggctcaggga |
| 631 | 221729 | tggaattagaccatttggcctttgaactttcataggaaaaatgacccaacatttcttagcatgagctacctcatctctagaa |
| | _at | gctgggatggacttactattcttgtttatattttagatactgaaaggtgctatgcttctgttattattccaagactgg |
| | | agataggcagggctaaaaaggtattattatttttcctttaatgatggtgctaaaattcttcctata |
| | | aaattccttaaaaataaagatggtttaatcactaccattgtgaaaacataactgttagacttcccgtttctgaaagaaaga |
| | | gcatcgttccaatgcttgttcactgttcctctgtcatactgtatctggaatgctttgtaatacttgcatgcttcttaga |
| | | ccagaacatgtaggtccccttgtgtctcaatactttttttttcttaattgcatttgttggctctattttaattt |
| 632 | 221730 | tagattccggtatatcgttcttcaagacacttgctctaagcggaatggaaatgtgggcaagactgtctttgaatatagaacac |
| | _at | agaatgtggcacgcttgcccatcatagatcttgctcctgtggatgttggcggcacagaccaggaattcggcgttgaaattg |
| | | ggccagtttgttttgtgtaaagtaagccaagacacatcgacaatgagcaccaccatcaatgaccaccgccattcacaag |
| | | aactttgactgtttgaagttgatcctgagactcttgaagtaatggctgatcctgcatcagcattgtatatatggtctt |
| | | aagtgcctggcctccttatccttcagaatatttattttacttacaatcctcaagttttaattgattttaa |
| | | atatttttcaatacaacagtttaggtttaagatgaccaatgacaatgaccacctt |
| 633 | 221731 | tttcagcaccgatggccatgtaaataagatgatttaatgttgattttaatcctgtatataaantaaaaagtncncaatgagttt |
| | _x_at | ngggcatatttaatgatgattatggagccttagaggtctttaatcattggttcnggctgcattatgtagtttaggctggaa |
| | | atggtttcacttgctctttgactgtcagcaagactgaagatggcttttcctggacagctagaaaacaca |
| | | aaatcttgtaggtcattgcacctatctcagccataggtgcagtttgcttctacatgatgctaaaggctgcgaatggga |
| | | tcctgatggaactaaggactccaatgtcgaactcttctttgctgcattcctttttcttcacttacaagaaaggcctgaat |
| | | ggaggacttttctgtaaccaggaacattttttaggggtcaaagtgctaataattaactcaaccaggtctactttttaatggc |
| | | tttcataacactaactcataaggttaccgatcaatgcatttcatacggatatagacctagggctctggagggtgggg |
| 634 | 221874 | ctggcctacgtgttcaattttctatgaacaaaggctttagtccttgacccagggctaaagtggtctgtccaagctgttgtt |
| | _at | tagagggagtatgataaaatgtttaaatctcatttggttaccttgagtcctggaacacgcagtaactgtcatgctat |
| | | ggagtcntcatctgtatttggctgggaatacaaatgaagattgtggtgtattcaagcagtagggtttttggaaatcgaat |
| | | cttttgtttttgttttagtgccaacaaaacttttttttgtctgactacattaaagataagactgactatatttatacaaca |
| | | gaaactttgtaatagattttttcagctttgttttttttcatcagggctggttggatttccttttaccctgtaatccaa |
| | | gcgttaatagtttgttagaagatgggttattgcatgtcactt |
| 635 | 223969 | gaagatcaaggatgttctcaacagtctagagtacagtccctctcctataagcaagaagctctcgtgtgctagtgtcaaaagccaa |
| | _s_at | ggcagaccgtcctcctgccctgctgggatggctgtcactggctgtgcttgtggctatggctgtggttcgtgggatgttcagct |
| | | ggaaaccacctgccactgccagtgcagtgtggtggactggaccactgcccgctgctgccacctgacctgacagggaggaggct |
| | | gagaactcagttttgtgaccatgacagtaatgaaaccagggtcccaaccaagaaatctaactcaaacgtcccacttcat |
| | | ttgttccattcctgattcttgggtaataaa |
| 636 | 223970 | ggagctcagagatctaagctgctttccatcttttctcccagccccaggacactgactctgtacaggatggggccgtcctcttg |
| | _at | cctccttctcatcctaatcccccttctccagctgatcaacccggggagtactcagtgttccttagactccgttatggata |
| | | agaagatcaaggatgactcaacagtctagagtacagtccctctcctataagcaa |
| | | gaagctctcgtgtgctagtgtcaaaagccaaggcagaccgtcctcctgccctgctgggatggctgtcactggctgtgcttgtggct |
| | | atggctgtggttcgtgggatgttcagctggaaaccacctgccactgccagtgcagtgtggtggactggaccactgcccgctgctg |
| | | ccacctgacctgacagggaggaggctgagaactcagttttgtgaccatgacagtaatgaaaccagggtcccaaccaagaaatcta |
| | | actcaaacgtcccact |
| 637 | 224724 | gcctgagaaagcaagcacgcactctcagtcaacatgacagattctggaggataaccagcaggagcagagataacttcaggaagtc |
| | _at | catttttgcccctgcttttgctttggattatacctcaccagctgcacaaaatgcattttttcgtatcaaaaagtcaccactaacc |
| | | ctcccccagaagctcacaaaggaaaacggagagagcgag |
| | | cgagagagatttccttggaaatttctcccaagggcgaaagtcattggaatttttaaatcataggggaaaagcagtcctgttctaa |
| | | atcctcttattcttttggtttgtcacaaagaaggaactaagaagcaggacagaggcaacgtggagaggctgaaaacagtgcagag |
| | | acgtttgacaatgagtcagtagcacaaaagagatgacatttacctagcactataaaccctggttgcctctgaagaaactgccttc |
| | | attgtatatatgtgactatttacatgtaatcaacatgggaacttttaggggaacctaataagaaatcccaattttcagga |
| | | gtggtggtgtcaataaacgctctgtggccagtg |
| 638 | 225664 | ggaacccagagctgctgtgtatttcgagcgggcagtttatcttttgctatacttattttcaattcaattacaccacgattcaaat |
| | _at | aattcccctcctaaaaccaaaaaggagggaaacgtcaactccattgcaattacttatcttcctcttctatctctgttatacgccg |
| | | gggcatagaatgctcgtatacatctctttaacaaccacaaaccttaagccatgtagatgaagttagtgcatcaacgggatacagt |
| | | tccatattgccttaaacctccttgttttagacacactaacatttataccaaattgcagattattctgcagagagggaattg |
| | | catgtttgtgttgta |
| 639 | 225681 | aattaatattcatcgcacttcttctgtggaaggactttgtgaaggaattggtgctggattagtggatgttgctatctgggttgg |
| | _at | cacttgttcagattacccaaaaggagatgcttctactggatggaattcagtttctcgcatcattattgaagaactaccaaaat |
| | | aaatgctttaattttcatttgctacctctttttttattatgccttggaatggttcacttaa |
| | | atgacattttaaataagtttatgtatacatctgaatgaaaagcaaagctaaatatgtttacagaccaaagtgtgatttcacact |
| | | gtttttaaatctagcattattcattttgcttcaatcaaaagtggtttcaatattttttttagttggttagaatactttcttcat |
| | | agtcacattctctcaacctataatttggaatattgttgtggtcttttgttttttctcttagtatagcatttttaaaaaaatat |
| | | aaaagctaccaatctttgtacaatttg |
| 640 | 225799 | aaatgactggatggtcgctgctttttaagtttcaaattgacattccagacaagcggtgcctgagcccgtgcctgtcttcagatc |
| | _at | ttcacagcacagttcctgggaaggtggagccaccagcctctccntgaataactgggagatgaaacaggaagctctatga |
| | | cacacttgatcgaatatgacagacacngaaaatcacgactcanccccctccagcacctctacctgttgcccgccgatca |
| | | cagccggaatgcagctgaaagattccctggggcctggttccaaccgcccactgtggactctgaggcctctgca |
| | | tttgcgggtggtctgcctgtgatattttggtcatgggctggtctg |
| 641 | 226237 | gaagaggagcaacatctatgccaaatactgtgcattctacaatggtgctaatctcagacctaaatgatactccatttaatttaaa |
| | _at | aaagagttttaaataattatctatgtgcctgtatttcccttttgagtgctgcacaacatgttaacatattagtgtaaaagcaga |
| | | tgaaacaaccacgtgttctaaagtctagggattgtgctataatccctattt |
| | | agttcaaaattaaccagaattcttccatgtgaaatggaccaaactcatattattgttatgtaaatacagagttttaatgcagta |
| | | tgacatcccacaggggaaaagaatgtctgtagtgggtgactgttatcaaatattttatagaatacaatg |
| | | aacggtgaacagactggtaacttgtttgagttcccatgacagatttgagacttg |
| 642 | 226248 | tgaaagtgggcatctctgcaggcacctgtactgccatcctgctcaccgtcttgacctgctacttttggaaaaagaatcaaaan |
| | _s_at | ctagagtacangtactccangctggtgatgaatgctactctcaaggactgnnnnntnncngnngctgacagct |
| | | gcgccatcatggnanncnnnnangnanagnnnnnnnncancnnnnnnc |
| | | anntgcgccatcatggaaggcgaggatgtagaggacgacctcatctttaccagcaagaagtcactctttgggaagatcaaa |
| | | tcatttacctccaagaggactcctgatggatttgactcagtgccgctgaagacatcctcaggaggcccagacatggacct |
| | | gtgagaggcactgcctgcctcacctgcctcctcaccttgcatagcacctttgcaagcctgcggcgatttgggtgccagc |
| | | atcctgcaacacccactgctggaaatctcttcattgtggccttatcagatgtttgaatttcagatctttttt |
| | | tatagagtacccaaaccctcctttctgcttgcctcaaacctgccaaatatacccacac |
| 643 | 226311 | aaacgacgcaaatctctgagctggggaccacttggagaaccggcttagtaacagtcctgatcttcgcaagccagcttcttctgc |
| | _at | atctgaggggctcctggcgcccagaggaggcagacagatgtcttctagctgagtttctaaccgcatgatgagactcagaccttc |
| | | cgctgcactagaaaatctgcaacagtgtccctgagtcacttctccttagtgggcagactcgtgttagatttgtggaacccagc |
| | | tctctgatttactccttttggaaaacccatggaatttcatgtataaggctttcatttgtattttaaggtttttctgtttg |
| | | ttttgagtatatacatggtgctcaatagcaacatcttagcagatgaagcagtttatgattccactccctcctgtatgacag |
| | | gtagccactatactgaatcaaggtgctgaactcaaatcacaaaattctggcttaccgatacaacaaccaatac |
| 644 | 226777 | tataaggtaactctttagtcctccatttagcacattaaatcctccaaagaataagtatcatgtgattattttagctttacaaaa |
| | _at | aaaaagttgaatggcgttttattttcatggcctataagcaggtaccttagtagggcagatataggaaaaacaaattagagcaa |
| | | aacaaatcctctacaaatccaaggcaggaaaagtggtggcagagtg |
| | | actcattctcctgtccctcccatcaggtcaaatcaggaggctgcagtgaatgcctgttctttgaatgtgtagcagttgttncc |
| | | tgtaactctttaaaacttggctataggctgtttagcacagtacagattaaagatacagttacgtaaacagcaaagtaat |
| | | tttatagtgcttcatccatttatcatgctttggtttgctaattttttcacatacctttttctatcacagtctgttg |
| | | cttttgtacacatttctcatattggggttcgaca |
| 645 | 226930 | ggtgccaggaaggtcacagatggacactggccattctggtcatctcagtctggaactcagtcccacttcttggcctggacaat |
| | _at | gaacaggattcagttttgctgttaactttgcttctctacttttttttgtttgtttgtaatagcacatcccagagacatcag |
| | | aaaccagcaactgattcagtgtgatttccagactttttaggcatgaaattcggacact |
| | | tcagtatttccaggaatagcatatgcacgctgttcttgcttcatggaatgctacatgctttctgtttttctcattttggatt |
| | | tctccaaaactaactgaatttaagcttcaggtccctttgtatgcagtagaaaggaattattaaaaacaccaccaaag |
| | | aaaataaatatatcctacttgaaatttactctatggacttacccactgctagaataaatgtatcaaatcttattt |
| | | gtaaattctcaattttgatatatatatgtatatatgcatatacatatccacacttgtctgcaag |
| 646 | 227140 | ttaccctctatttaaatgctttgaaaaacagtgcattgacaatgggttgatatttttctttaaaagaaaaatataattat |
| | _at | gaaagccaagataatctgaagcctgattattttaaaactttttatgttctgtggttgatgttgtttgtttgtttgtt |
| | | tctattttgttggttttttactttgttttttgttagttttgttttgttttgcatactacatgcagttctttaaccaatgt |
| | | ctgtttggctaatgtaattaaagttgaaatttatatgagtgcatttcaactatgtcaatggtttcttaatatttattgt |
| | | gtagaagtactggtaatttttttatttacaatatgtttaaagagataacagtttgatatgttttcatgtgtttatagc |
| | | agaagttatttatttctatggcattccagcggatattttggtgtttgcgaggcatgcagtcaatattttgtacagtta |
| | | gtggacagtattcagcaacgcctgatagcttctttggcctt |
| 647 | 227566 | ccacacaccaggcactaatcacctggtgaggatttggcatatccaccaaaaaatgcatccgatttaaccaacatctccaccagc |
| | _at | gctacggactcctcccaattctgacatctcttgcagacaatactatgctctctacacactgtttagaaatggaaaggtgatc |
| | | tgcactgtatcttgggtttgttggctatgcttcctttgatgacatatattatacagtatatatatacatatattttttttgtt |
| | | agagttctagccattttatttctccgcagggtcctttctcagacattactgcatgctgtatatggcgttagctgtgtgttga |
| | | tcttctaaaagatgatagagtttactggtaattgtgtaatcagctcctgcctttttatt |
| 648 | 227676 | caggaaactcttctctgacttggggagttcctacgcganacaactgggcttccgggacagctgggtcttcataggagccaaaga |
| | _at | cctcaggggtaaaagcccctttgagcagttcttaaagaacagcccagacacaaacaaatacgagggatggccagagctgc |
| | | tggagatggagggctgcatgcccccgaagccattttagggtg |
| | | gctgtggctcttcctcagccaggggcctgaagaagctcctgcctgacttaggagtcagagcccggcaggggctgaggaggagg |
| | | agcagggggtgctgcgtggaaggtgctgcaggtccttgcacgctgtgtcgcgcctctcctcctcggaaacagaaccctccc |
| | | acagcacatcctacccggaagaccagcctcagagggtccttctggaaccagctgtctgtggagagaatggggtgctttcg |
| | | tcagggactgctgacggctggtcctgaggaaggacaaactgcccagacttgagcc |
| 649 | 227719 | cacttgatagactgtaagcacctgcttaactttgtgtcccaaatatttagtgtgtatatatatatatatatanncncacacac |
| | _at | acacatatatattcaacaaataaagcaaaatataacatgcatttcacattttgtctttccctgttacgattttaatagcagaa |
| | | ctgtatgacaagtttaggtgatcctagcatatgttaaattcaaattaatgtaaaacagattaacaacaacaaagaaactgt |
| | | ctatttgagtgaagtcatgctttctattataataacttggcttcggttatccatcaaatgcacacttatactgttatctg |
| | | attg |
| 650 | 229218 | gccaaaaggcctggcttctgataggaaactggtaagaaactcttcatgaaaacacatcactaatattcgctattactctcctgg |
| | _at | tctgaagtcagcttttctgaaccattaaggtatttcatcacaagttatattttataatatcagtttaagaggct |
| | | tttattcatgtgaacaccagnnccctttcaggggcatggtctttttgaaaaaaaaaaaanaa |
| | | aaaaannaacagttttagccacatatcagatatttctatatctaattatcctttatggctaacattctgcctccattgttaa |
| | | ggtataattgttcctgaatttaaaggtggtttggcctctaatttaattctgattcagactctcctgtcaggactca |
| | | agaaaatttaattaattaccaaggattaagtcttctggttaaggtttctgggaaaaaaaaatagcaaagat |
| | | gttgatttcttggaatcctttacaggttcataacagaaaaatcttcattccctgtaggcatttaattaaacctagttga |
| | | gaagtgtgtgggattcctcaattatgaacaaaacacgtatattggctttcttt |
| 651 | 229802 | gacatgattgtctataatctcgctagccttgtactgtgtgtgcatagcaattacagggaagtaatctagctcctgactattat |
| | _at | gttgaactatgtcgctgctttttacaaacttgtcttgatccaaagcagtcacaatgataaccctgcatatctggg |
| | | aatcataagtcaactatgtatctctgtgtgtgtatatatatgtatgtatgtatctattttcaaactgt |
| | | gatttaatatttaaatattcctactgccatttttgtgactgaaaaactacacatgaggaaacgtcttagaatatccaataga |
| | | ggaaaaataacacttgggcaatctgtcatgtttcacaacagttctcatttttctcatgatttgtgtagcgtgg |
| | | aatgtgtttgctcaatgtgaagggttttcattgctcaatttctctgtgtaa |
| 652 | 231766 | ggttccggctaacacattttctaagtcgccagtgctgcttacagtttgaatacatgaaaatcctgtttctnagatgtttgcg |
| | _s_at | cacgtgcttattaggaaatgagtctgtatggaaatctcaccacagataatggttaacgaaccgggtcgacatcacaaag |
| | | gagggtggagactctttttactaacttgaatgagacaaaagcagtggtgtcagtttataatcctgatgcatttcagtaata |
| | | atgtagaaaaacattattttaaaaaagttccaacacacagccatgaggagccnnnnnannantcagttttgaaagaggtg |
| | | cataataaaactactaaccagaggagtctatgccatttt |
| 653 | 231832 | gagtttcaactttaaatgttcactatgtcatttagtgtccanctttacggataggttgactatctaaataggcatttttagt |
| | _at | cattaaaaaaaantctagtcaccaggaggatccctataactcaaaataacttgtttgtaaaagaaaatttgtttactta |
| | | cccattagtaagttcctgcatattcattataagatggcaaatcaaacttttctaggatgaagacagcttatttttaagt |
| | | tgtatagtcttagttggtttagggtctcaattttaattaataaaatacttggtttttatttgcttgtccttttgaattcct |
| | | gttttaataattttaaaatgagcacaaagaangttgaagttcagattaatctcttctgaa |
| | | tgatgtttttttcctctgtgatgagttgtttctg |
| 654 | 231879 | tgctgtagtcactggtgttcctcacccaccagctgtaactcagtttgtgtgaggtacagccacagaagatgtcatgtactgta |
| | _at | tattacctggtgatagttgcttttcaccccccgagttcagtttctaggagccaatgaaacttcccctcacctcctcatctt |
| | | tccaagttgttctttgaattgaggagtttgaaggcataaacagttacttggggatttgcgaaaatcctacttagttac |
| | | tgcgtttacagttctttggcccagtctctgacccttcccnagtatttgtgcnatgattg |
| | | tgtttactgctggatttttgaaggttttttttttnnaagaaagtgccatttcattatttgatt |
| 655 | 232176 | gggatcactgggagaagccatggcattatcttcaggcaatttagtctgtcccaaataaaataaatccttgcatgtaaatcattca |
| | _at | agggttatagtaatatttcatatactgaaaagtgtctcataggagtcctcttgcacatctaaaaaggctgaacatttaagtatcc |
| | | cgaattttcttgaattgctttccctatagattaattacaattggatttcatc |
| | | atttaaaaaccatacttgtatatgtagttataatatgtaaggaatacattgtttataaccagtatgtacttcaaaaatgtgtatt |
| | | gtcaaacatacctaactttcttgcaataaatgcaaaagaaactggaacttgacaattataaatagtaatagt |
| | | gaagaaaaaatagaaaggttgcaattatataggccatgggtggctcaaaactttgaa |
| 656 | 232458 | gattcctggattgatgtcttatctacaggcttctttttaaaaatnnnnnnngattggctgtgagtttaccaggattatagttga |
| | _at | ggctaaaggacagctcctcaggaaagcccctgttctacatctacggtcacatgctggaccttgagttgtcactcagagaaaag |
| | | agtgccatctaccgaaactccacagtttccattgtgaatggcttctttggt |
| | | gcagagttccaaaaattatgtagcccagctctttaattttgtaacatctaatgatatcaccgccttgaagtgattaaagtaga |
| | | ttgcttaaagaattaaagctttaaagatgaaagatgttattgcttgctggacatgaggaacagttgtaaagttt |
| | | ccaggtctacaataactttctgganccctctcagtgaactgtttcttgta |
| 657 | 232481 | gaagtccatcctttggtccaaagcatctggaagaggaagaagagaggaatgagaaagaaggaagtgatgcaaaacatctccaa |
| | _s_at | agaagtcttttggaacaggaaaatcattcaccactcacagggtcaaatatgaaatacaaaaccacgaaccaatcaacagaat |
| | | ttttatccttccaagatgccagctcattgtacagaaacattttagaaaaagaaagggaacttcagcaactgggaatcacag |
| | | aatacctaaggaaaaacattgctcagctccagcctgatatggaggcacattatcctggagcccacgaagagctgaagttaat |
| | | ggaaacattaatgtactcacgtccaaggaaggtattagtggaacagacaaaaaatgagtattttgaacttaaagctaattta |
| | | catgctgaacctgactatttagaagtcctggagcagcaaacatagatggagagtttgagggctttcgcagaaatgctgt |
| | | gattctgttttaagtccataccttgtaaataagtgccttacgtgagtgtgtcatcaatcagaacctaagc |
| 658 | 233555 | gctgctcaagcgcctgcagaacaacgacacgtgcagcatgccaggcctcacgtgcttcacccacgacaaccagcactggca |
| | _s_at | gacggcgcctttctggacactggggcctttctgtgcctgcaccagcgccaacaataacacgtactggtgcatgaggacca |
| | | tcaatgagactcacaatttcctcttctgtgaatttgcaactggcttcctagagtactttgatctcaacacagaccc |
| | | ctaccagctgatgaatgcagtgaacacactggacagggatgtcctcaaccagctacacgtacagctcatggagctg |
| | | aggagctgcaagggttacaagcagtgtaacccccggactcgaaacatggacctgg |
| 659 | 234994 | acaatcggctaaccttgacatttctttttaccttcatatgccactatctcggtagttcaaaaaaatttagttcttgataaatt |
| | _at | gccttgaagtttaccttgtgctggagagccttatgataactccaaagactttcttacggtataatacatgttg |
| | | tttaggattgtgtttcttagtcactgaagataataaatattaaaatggatgttttcatcagaaaattttcat |
| | | gttttcctttaaggtaacataattgtaagaattgtttaataaaatactcaggaaattctaaaggtttctccnaatacctaaac |
| | | atttctgaacatcagtattgcagttgtggaagagcagaaggaggatacatttgtttgtgttgctccccaa |
| | | aattccaccttgcatttgcatcacaaacttccctcaattgaggcagttttctttg |
| 660 | 235976 | cagtgctgctgtgaactaaagtatgtcatttatgctcaaagtttaattcttcttcttgggatattttaaaaatgctactgag |
| | _at | attctgctgtaaatatgactagagaatatattgggtttgctttatttcataggcttaattctttgtaaatctgaatg |
| | | accataatagaaatacatttcttgtggcaagtaattcacagttgtaaagtaaataggaaaaattatttt |
| | | atttttattgatgtacattgatagatgccataaatcagtagcaaaaggcacttctaaaggtaagtggtttaagttgcctcaa |
| | | nagagggacaatgtagctttattttacaagaaggcatagttagatttctatgaaatatttattctgtac |
| | | agttttatatanttttggttcacaaaagtaattattcttgggtgcctttcaa |
| 661 | 236894 | aaagtatattgtgctagcttgtctaagaataaacttnnatactgttgggggagggctgcacctgtcaagataacctgtcaat |
| | _at | gtagtaggaaaacaggaggggacagtaacagaaaagcacgggaaaagatggcaaggttagttaaaatagaaaagtgctcagt |
| | | tcctcatacctgtaatcccagcagtttagggggccaaggaaggtgggtcacttgagcccaagagttcaaggccat |
| | | cctgggcaatgtggcgaaagtgtctacaaaaaaatacaaaaagaggaagaaatgatatttcacaagtttgta |
| | | tcatttgtcat |
| 662 | 237521 | cagtccaaaatccatagacacagacagtagaaaggtggttatncaagggnctggtaggagagggagttagtatttagtggta |
| | _x_at | tagagttttagtgtttnccgggtaaaaatgttnctagagatctgttgcaaccatgtgaatatacttaacactacc |
| | | gtactgtacactgaaaaaatggttaagaggataaatttaaatggttaagaggaaaaatggttaag |
| | | aggataatgttgttttgttaccacagtagtaacttttaaaaaccctcatgtatgttcttt |
| 663 | 37892_ | caacccattttgtgccacatgcaagttttgaataaggatggtatagaaaacaacgctgcatatacaggtaccatttaggn |
| | at | annancngatgcctttntgggggcagaatcacatggcaaaagctttgaaaatcataaagatataagttggtgtggct |
| | | aagatggaaacagggctgattcttgattcccaattctcaactctccttttcctatttgaatttctttggtgctgtaga |
| | | aaacaaaaaaagaaaaatatatattcataaaaaatatggtgctcattctcatccatccaggatgtactaaaacagtgtgt |
| | | ttaataaattgtaattattttgtgtacagttctatactgttatctgtgtccatttccaaaacttgcacgtgtccctgaattcc |

TABLE 7
MSP primers and PCR conditions

| | | | | | Re-anneal |
|---|---|---|---|---|---|
| Gene | Probe set | Gene Name | Forward primer 5′-3′ | Reverse primer 5′-3′ | Temp |
| CA4 | 206208_at | Carbonic anhydrase IV | TCGTTTTTCGCGTTTAGTTGTC | GCGCACCGAAAAAACCG | 61.0° C. |
| | | | (SEQ ID NO: 664) | (SEQ ID NO: 665) | |
| CXCL12 | 209687_at | Chemokine, CXC | CGGTTACGGTTAGTATTCGGTTTC | AAAATACGACGATAAAAAACG | 64.0° C. |
| | | Motif, Ligand 12 | (SEQ ID NO: 666) | CG | |
| | | | | (SEQ ID NO: 667) | |
| DF | 205382_ | complement factor D | CGAGGGTTTTTTAGCGATTTGTC | AAACGAACCGCTCCCCG | 64.0° C. |
| | s_at | | (SEQ ID NO: 668) | (SEQ ID NO: 669) | |
| MAMDC2 | 228885_at | MAM domain-containing | TTCGGCGTTTTCGTTTTTTAC | CCCCTTAACAACATAATCGCG | 60.0° C. |
| | | protein 2 precurosr | (SEQ ID NO: 670) | (SEQ ID NO: 671) | |
| MT1M | 217546_at | Metallothionein-1M | GATGGTGCGTTCGGTATTTATGT | GCTTACACCCGCCCGACTA | 62.0° C. |
| | | | (SEQ ID NO: 672) | (SEQ ID NO: 673) | |
| CAGE | | Control unbiased | GTTTTGTGATTGGTTAGGTTATT | CCRTCACCTTCTACACCAAAA | 61.0° C. |
| | | amplification assay | AAAGT | AATA | |
| | | | (SEQ ID NO: 674) | (SEQ ID NO: 675) | |

TABLE 8
COBRA Primers and PCR conditions

| | | | | | Re-anneal |
|---|---|---|---|---|---|
| | | | | | Temp, |
| Gene | Probe set | Gene Name | Forward primer 5′-3′ | Reverse primer 5′-3′ | Enzyme |
| ADAMDECI | 206134_at | ADAM-like, decysin 1 | TGTGGATTTATTTTTATAAG | ATAACTCATTAAAAACTCAC | 61.0° C. |
| | | | GATATTGAAT | ATCTAAACACTA | BstU I |
| | | | (SEQ ID NO: 676) | (SEQ ID NO: 677) | |
| GPM6B | 209170_s_ | Neuronal membrane | TTAAGAAAGAGAGTAAGGAG | AAAATCCCAATTAAAAAACA | 6O.0° C. |
| | at | glycoprotein M6-b | GAAGAGTT | ACCA | Hinf I |
| | | | (SEQ ID NO: 678) | (SEQ ID NO: 679) | |
| P2RY14 | 206637_at | Purinergic receptor | CAAAATAATAAATCCCTCTA | GGAGGAAAGGAATTAGTTTA | 60.0° C. |
| | | P2Y, G-protein coupled, | CTACTATTATCAA | GAAGTTAGTT | Acl I |
| | | 14 | (SEQ ID NO: 680) | (SEQ ID NO: 681) | |

### BIBLIOGRAPHY

Affymetrix. GeneChip expression data analysis fundamentals. Affymetrix, Santa Clara, Calif. USA, 2001.
Alon et al., Proc. Natl. Acad. Sci. USA: 96, 6745-6750, June 1999
Ausubel, F. et al., “Current Protocols in Molecular Biology”, John Wiley & Sons, (1998)
Bonner et al (1973) J. Mol. Biol. 81:123
Clark et al. 2006, Nature Protocols 1:2353-2364
DeRisi, et al., Nature Genetics 14:457-460 (1996)
Germer et al., Genome Res. 10:258-266 (2000)
Guo et al., Nucleic Acids Res. 22:5456-5465 (1994)
Heid et al., Genome Res. 6:986-994 (1996)
Hubbell E. W., W. M. Liu, and R. Mei. Robust estimators for expression analysis. Bioinformatics, 18:1585-1592, 2002.
Irizarry R W., B. M. Bolstad, F. Collin, L. M. Cope, B. Hobbs, and T. P. Speed. Summaries of affymetrix genechip probe level data. Nucleic Acid Research, 31, 2003.
Kraus, M. and Aaronson, S., 1991. Methods Enzymol., 200:546-556
Maskos and Southern, Nuc. Acids Res. 20:1679-84, 1992
Moore et al., BBA, 1402:239-249, 1988
Nielsen (1999) Curr. Opin. Biotechnol. 10:71-75
Nielsen et al. (1991) Science 254: 1497-1500
Pease et al., Proc. Natl. Acad Sci. USA 91(11):5022-5026 (1994)
Pevzner et al., J. Biomol. Struc. & Dyn. 9:399-410, 1991
Schena, et al. Science 270:467-470 (1995)
Smith et al., Science 258:1122-1126 (1992)
Smyth G. K. Bioinformatics and Computational Biology Solutions using R and Bioconductor. Springer, New York, 2005.
Smyth G. K. Linear models and empirical bayes methods for assessing differential expression in microarray experiments. Statistical Applications in Genetics and Molecular Biology, 3(1): Article 3, 2004.
T. Sano and C. R. Cantor, Bio/Technology 9:1378-81 (1991)
Urdea et al., Nucleic Acids Symp. Ser., 24:197-200 (1991)
Wedemeyer et al., Clinical Chemistry 48:9 1398-1405, 2002)
Weissleder et al., Nature Medicine 6:351-355, 2000

1.-35. (canceled)

36. A method comprising
(i) amplifying DNA from a blood sample of a human to obtain amplified DNA comprising the promoter region of the SDC2 gene, wherein said amplifying comprises bisulfite conversion of the blood DNA;
(ii) detecting the level of methylation in said promoter region of the SDC2 gene in the bisulphite-converted DNA.

37. The method according to claim 36 wherein said human is suffering from an adenoma or an adenocarcinoma.

38. The method according to claim 36 wherein said human is suffering from a colorectal neoplasm.

39. The method of claim 36 further comprising
(iii) comparing the level of methylation detected in (ii) to the level of methylation in a control DNA from a non-neoplastic human; and
(iv) identifying a higher level of methylation in the DNA from the blood sample in
(iii) as compared to the level of methylation in the control DNA in (iii);
wherein the higher level of methylation in the DNA from the blood sample relative to the level of methylation of the control DNA is indicative of a large intestine neoplasm or a predisposition to the onset of a large intestine neoplasm.

40. The method of claim 39, wherein said amplifying comprises amplifying CpG sites in said promoter region of the SDC2 gene using primers that hybridize to methylated CpG nucleotides positions in bisulphite-treated genomic DNA.

41. The method according to claim 39 wherein said large intestine neoplasm is an adenoma or an adenocarcinoma.

42. The method according to claim 40 wherein said large intestine neoplasm is an adenoma or an adenocarcinoma.
